(19) United States
(12) Patent Application Publication
(10) Pub. No.: US 2016/0106102 A1
(43) Pub. Date: Apr. 21, 2016

(54) KNOWN AND NEW HETEROCYCLIC COMPOUNDS AS PEST CONTROL AGENTS

(71) Applicant: BAYER CROPSCIENCE AKTIENGESELLSCHAFT, Monheim am Rhein (DE)

(72) Inventors: Susanne KUEBBELER, Duesseldorf (DE); Markus HEIL, Leichlingen (DE); Pierre-Yves COQUERON, Lyon (FR); Johannes-Rudolf JANSEN, Monheim (DE); Reiner FISCHER, Monheim (DE); Roland ANDREE, Langenfeld (DE); Birgit BOLLENBACH-WAHL, Weiler/Bingen (DE); Eva-Maria FRANKEN, Leverkusen (DE); Kerstin ILG, Koeln (DE); Arnd VOERSTE, Koeln (DE)

(21) Appl. No.: 14/892,294

(22) PCT Filed: May 23, 2014
(86) PCT No.: PCT/EP2014/060595

§ 371 Date: Nov. 19, 2015

(30) Foreign Application Priority Data
May 23, 2013 (EP) 13168871.5

(51) Int. Cl. A01N 43/90; A01N 43/56; C07D 401/12; C07D 403/12; A01N 43/58; A01N 43/713; A01N 43/647; A01N 43/653; A01N 43/66; C07D 487/04; C07D 513/04; C07D 413/14; A01N 43/76; C07D 417/14; A01N 43/78; A01N 43/82; C07D 471/04; C07D 401/14

(52) U.S. Cl. CPC A01N 43/90; C07D 401/14; A01N 43/56; C07D 401/12; C07D 403/12; A01N 43/58; A01N 43/713; A01N 43/647; A01N 43/653; A01N 43/66; C07D 487/04; C07D 513/04; C07D 413/14; A01N 43/76; C07D 417/14; A01N 43/78; A01N 43/82; C07D 471/04

(57) ABSTRACT

The present application relates to the use of known and novel heterocyclic compounds for controlling animal pests, to novel heterocyclic compounds, to processes for preparation thereof and to the use thereof for controlling animal pests.

The present application relates to the use of known and novel heterocyclic compounds for controlling animal pests, to novel heterocyclic compounds, to processes for preparation thereof and to the use thereof for controlling animal pests.

WO 2011/049987 A2, WO 2011/106114 A1, WO 2009/025793 A2, US 2008/0103182 A1, WO 2007/055941 A2, WO 2006/114313 A1, US 2009/0163545 A1 and FR 2836915 A1 disclose heterocyclic compounds for which pharmaceutical uses are described. U.S. Pat. No. 2,511,231 and JP 63218665 A disclose heterocyclic compounds for use in photography. The literature describes particular substituted pyridines as insecticides; see, for example WO 2003/097604 A1. WO 2005/074686 A1 and WO 2008/014905 A2 disclose carboxamides for control of unwanted microorganisms.

The heterocyclic compounds of the formulae (G1), (G2) and (G4) are known. No use for these compounds has been described.

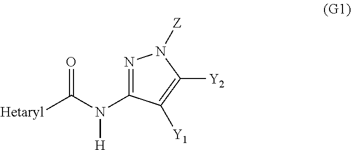

| No. | CAS number | Hetaryl | Y₁ | Y₂ | Z |
|---|---|---|---|---|---|
| G1-1 | 1394594-59-2 | 6-cyanopyridin-3-yl | 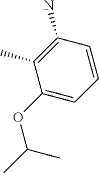 | | methyl |
| G1-2 | 1388443-85-3 | 5-bromopyridin-3-yl | H | H | 3-amino- |
| | | | | | 3-oxopropyl |
| G1-3 | 1386349-79-6 | 5-chloro-6- | H | H | methyl |
| | | isopropoxypyridin-3-yl | | | |
| G1-4 | 1386201-75-7 | 5-chloro-6-(2,2,2- | H | H | benzyl |
| | | trifluoroethoxy)- | | | |
| | | pyridin-3-yl | | | |
| G1-5 | 1378162-64-1 | 2-ethylpyrimidin-5-yl | 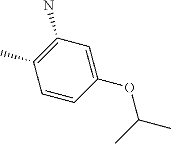 | | 2,2,2- trifluoroethyl |
| G1-6 | 1378144-60-5 | 2-methylpyrimidin-5-yl | 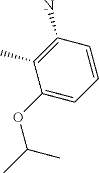 | | 4-fluorophenyl |
| G1-7 | 1364005-12-8 | pyridin-3-yl | H | H | cyanomethyl |
| G1-8 | 1343727-78-5 | 6-fluoropyridin-3-yl | H | H | methyl |
| G1-9 | 1331064-54-0 | 6-methylpyridin-3-yl | H | H | methyl |
| G1-10 | 1322107-71-0 | 5-chloro-6-(2,2,2- | H | H | phenyl |
| | | trifluoroethoxy)-pyridin- | | | |
| | | 3-yl | | | |
| G1-11 | 1311484-14-6 | 6-methylpyridin-3-yl | H | H | quinolin-8- |
| | | | | | ylmethyl |
| G1-12 | 1301300-24-2 | 6-(2,2,2- | H | H | quinolin-8- |
| | | trifluoroethoxy)pyridin- | | | ylmethyl |
| | | 3-yl | | | |
| G1-13 | 1299334-31-8 | 6-(2,2,2- | H | H | pyridin-4- |
| | | trifluoroethoxy)-pyridin- | | | ylmethyl |
| | | 3-yl | | | |
| G1-14 | 1298991-86-2 | 5-bromopyridin-3-yl | H | H | 2-(pyridin-2- |
| | | | | | yl)ethyl |
| G1-15 | 1293710-76-5 | 5-chloro-6- | H | H | pyridin-2- |
| | | isopropoxypyridin-3-yl | | | ylmethyl |
| G1-16 | 1293710-71-0 | 5-chloro-6-(2,2,2- | H | H | pyridin-3- |
| | | trifluoroethoxy)pyridin- | | | ylmethyl |
| | | 3-yl | | | |
| G1-17 | 1293302-73-4 | pyridin-3-yl | 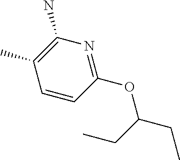 | | methyl |
| G1-18 | 1288605-17-3 | 6-chloropyridin-3-yl | H | H | quinolin-8- |
| | | | | | ylmethyl |
| G1-19 | 1252297-97-4 | 6-(2,2,2- | H | H | pyridin-2- |
| | | trifluoroethoxy)pyridin- | | | ylmethyl |
| | | 3-yl | | | |
| G1-20 | 1193169-23-1 | 6-chloropyridin-3-yl | H | methyl | phenyl |
| G1-21 | 1193168-56-7 | 6-chloropyridin-3-yl | H | H | 4-chlorophenyl |
| G1-22 | 1193166-26-5 | 6-chloropyridin-3-yl | H | ethyl | phenyl |
| G1-23 | 1192992-75-8 | 6-chloropyridin-3-yl | H | ethyl | 4-cyanophenyl |
| G1-24 | 1192974-12-1 | 6-chloropyridin-3-yl | H | methyl | 3-chlorophenyl |
| G1-25 | 1192974-02-9 | 6-chloropyridin-3-yl | H | methyl | 4-chlorophenyl |
| G1-26 | 1192967-85-3 | 6-chloropyridin-3-yl | H | H | 2,4- |
| | | | | | difluorophenyl |
| G1-27 | 1189895-78-0 | 6-chloropyridin-3-yl | H | ethyl | 4-chlorophenyl |
| G1-28 | 1189688-16-1 | 6-chloropyridin-3-yl | H | ethyl | 2,4- |
| | | | | | difluorophenyl |
| G1-29 | 1189644-39-0 | 6-chloropyridin-3-yl | H | ethyl | 3-methylphenyl |
| G1-30 | 1189486-79-0 | 6-chloropyridin-3-yl | H | methyl | 4- |
| | | | | | isopropylphenyl |
| G1-31 | 1189418-28-7 | 6-chloropyridin-3-yl | H | | 4- |
| | | | | | isopropylphenyl |
| G1-32 | 1185159-42-5 | 6-chloropyridin-3-yl | H | methyl | 3,4- |
| | | | | | dichlorophenyl |
| G1-33 | 1185131-97-8 | 6-chloropyridin-3-yl | H | H | phenyl |
| G1-34 | 1185105-88-7 | 6-chloropyridin-3-yl | H | H | 3-chlorophenyl |
| G1-35 | 1185047-56-6 | 6-chloropyridin-3-yl | H | ethyl | 3,4- |
| | | | | | dichlorophenyl |
| G1-36 | 1184979-44-9 | 6-chloropyridin-3-yl | H | | 4- |
| | | | | | methoxyphenyl |
| G1-37 | 1184968-09-9 | 6-chloropyridin-3-yl | H | methyl | 3- |
| | | | | | methylphenyl |
| G1-38 | 1179419-36-3 | pyridin-3-yl | bromine | H | 2-fluorobenzyl |
| G1-39 | 1172876-67-3 | 6-chloropyridin-3-yl | H | ethyl | 3-chlorophenyl |
| G1-40 | 1172816-42-0 | pyridin-3-yl | H | H | 2,4- |
| | | | | | difluorophenyl |
| G1-41 | 1052597-55-3 | 5-bromopyridin-3-yl | H | H | methyl |
| G1-42 | 1018036-37-7 | pyridin-3-yl | H | H | 2-fluorobenzyl |
| G1-43 | 1018035-68-1 | pyridin-3-yl | H | H | 4-fluorobenzyl |
| G1-44 | 1018034-07-5 | pyridin-3-yl | H | H | methyl |
| G1-45 | 1018020-80-8 | pyridin-3-yl | H | H | 3-fluorobenzyl |
| G1-46 | 1017985-14-6 | pyridin-3-yl | H | H | ethyl |
| G1-47 | 1017984-07-4 | pyridin-3-yl | H | H | 4-methylbenzyl |
| G1-48 | 1017981-75-7 | pyridin-3-yl | bromine | H | 3-methylbenzyl |
| G1-49 | 1017980-53-8 | pyridin-3-yl | bromine | H | 4-methylbenzyl |
| G1-50 | 1017978-97-0 | pyridin-3-yl | bromine | H | 4-fluorobenzyl |
| G1-51 | 1017586-40-1 | pyridin-3-yl | H | H | 2,5- |
| | | | | | dichlorobenzyl |
| G1-52 | 1017534-13-2 | pyridin-3-yl | H | H | 2-methylbenzyl |
| G1-53 | 1017532-86-3 | pyridin-3-yl | H | H | 2-chlorobenzyl |
| G1-54 | 1006331-96-9 | pyridin-3-yl | H | methyl | benzyl |
| G1-55 | 1005882-11-0 | 6-chloropyridin-3-yl | H | H | 4-chlorobenzyl |
| G1-56 | 1001841-69-5 | pyridin-3-yl | cyano | ethylsulphanyl | phenyl |
| G1-57 | 1001841-28-6 | pyridin-3-yl | cyano | methyl-sulphanyl | phenyl |
| G1-58 | 957513-83-6 | pyridin-3-yl | H | methyl | 3-methylbenzyl |
| G1-59 | 957501-08-5 | pyridin-3-yl | H | H | 4-chlorobenzyl |
| G1-60 | 957336-71-9 | pyridin-3-yl | H | H | 2-ethoxy-2- |
| | | | | | oxoethyl |
| G1-61 | 957277-00-8 | pyridin-3-yl | H | methyl | 4-fluorobenzyl |
| G1-62 | 957007-89-5 | 6-chloropyridin-3-yl | H | H | methyl |
| G1-63 | 957007-68-0 | 6-(2,2,2-trifluoroethoxy)- | H | H | methyl |
| | | pyridin-3-yl | | | |
| G1-64 | 956962-14-4 | pyridin-3-yl | H | H | 1- |
| | | | | | naphthylmethyl |
| G1-65 | 956763-30-7 | pyridin-3-yl | H | H | 2,4- |
| | | | | | dichlorobenzyl |
| G1-66 | 956627-03-5 | pyridin-3-yl | H | methyl | 4-methoxy-4- |
| | | | | | oxobutyl |
| G1-67 | 956536-22-4 | 6-methoxypyridin-3-yl | H | H | methyl |
| G1-68 | 956334-28-4 | 5,6-dichloropyridin-3-yl | H | H | methyl |
| G1-69 | 955963-35-6 | pyridin-3-yl | H | H | benzyl |
| G1-70 | 895950-13-7 | pyridin-3-yl | chlorine | H | 2-chloro-6- |
| | | | | | fluorbenzyl |
| G1-71 | 1401567-88-1 | pyridin-3-yl | 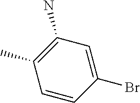 | | H |
| G1-72 | 1398113-90-0 | 6-methylpyridin-3-yl | H | cyclohexyl-methyl | H |
| G1-73 | 1390185-62-2 | 5-bromopyridin-3-yl | methyl | H | H |
| G1-74 | 1373059-58-5 | 5-bromopyridin-3-yl | H | cyclopentyl | H |
| G1-75 | 1355894-01-7 | 6-(2,2,3,3- | H | H | H |
| | | tetrafluoropropoxy)- | | | |
| | | pyridin-3-yl | | | |
| G1-76 | 1346686-04-1 | 6-isopropoxypyridin-3-yl | H | cyclohexyl-methyl | H |
| G1-77 | 1342817-43-9 | 6-methoxypyridin-3-yl | H | cyclohexyl-methyl | H |
| G1-78 | 1291744-04-1 | 6-(trifluoromethyl)- | H | H | H |
| | | pyridin-3-yl | | | |
| G1-79 | 1282798-05-3 | 5-bromopyridin-3-yl | 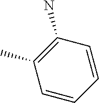 | | H |
| G1-80 | 1275713-61-5 | 6-chloropyridin-3-yl | 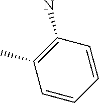 | | H |
| G1-81 | 1275068-38-6 | 6-methylpyridin-3-yl | 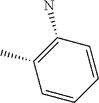 | | H |
| G1-82 | 1270883-15-2 | pyridin-3-yl | 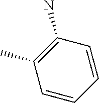 | | H |
| G1-83 | 1249829-04-6 | 6-chloropyridin-3-yl | H | H | H |
| G1-84 | 1249637-58-8 | 6-methoxypyridin-3-yl | H | H | H |
| G1-85 | 1248794-65-1 | pyridin-3-yl | H | H | H |
| G1-86 | 1248082-67-8 | 6-methylpyridin-3-yl | H | H | H |
| G1-87 | 1248016-37-6 | 5,6-dichloropyridin-3-yl | H | H | H |
| G1-88 | 1247118-66-6 | 5-bromopyridin-3-yl | H | H | H |
| G1-89 | 1241269-40-8 | 5-chloro-6- | H | H | H |
| | | isopropoxypyridin-3-yl | | | |
| G1-90 | 1042684-17-2 | pyridin-3-yl | cyano | ethylsulphanyl | H |
| G1-91 | 942662-36-4 | pyridin-3-yl | cyano | methylsulphanyl | H |
| G1-92 | 313371-16-3 | 5-bromopyridin-3-yl | H | OH | phenyl |
| G1-93 | 957010-63-8 | pyridin-3-yl | H | H | 3-methylbenzyl |
| G1-94 | 1436033-05-4 | pyridin-3-yl | H | H | 1-phenylethyl |
| G1-95 | 1445574-69-5 | 5-iodopyridin-3-yl | H | methyl | methyl |
| G1-96 | 1458185-22-2 | 6-methoxypyridin-3-yl | 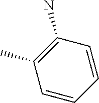 | | H |
| G1-97 | 1458685-50-1 | 5,6-dichloropyridin-3-yl | 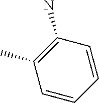 | | H |
| G1-98 | 1479443-93-0 | 5-methylpyridin-3-yl | 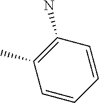 | | H |
| G1-99 | 1486844-11-4 | 5-methylpyridin-3-yl | H | H | H |
| G1-100 | 1486514-55-9 | pyridin-3-yl | H | 1-methylbutyl | H |
| G1-101 | 1509673-22-6 | 6-bromopyridin-3-yl | H | H | methyl |
| G1-102 | 1512341-63-7 | 6-bromopyridin-3-yl | H | H | H |

| | | | | |
|---|---|---|---|---|
| G1-103 | 1522750-95-3 | 6-bromopyridin-3-yl | 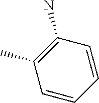 | H |

In the case of compounds G1-1, G1-5, G1-6, G1-17, G1-71, G1-79 to G1-82 and G1-96 to G1-98 and G1-103, the Y₁ and Y₂ columns also show the carbon atoms bonded to Y₁ and Y₂ and the nitrogen atom bonded to Z. The same applies to all the tables in this patent application in which Y₁ and Y₂ occur together.

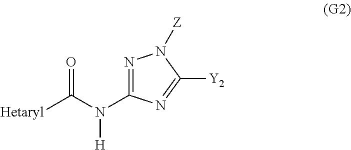

| No. | CAS number | Hetaryl | Y₂ | Z |
|---|---|---|---|---|
| G2-1 | 1390344-07-6 | 6-methylpyridin-3-yl | H | (benzylamino)- |
| | | | | 2-oxoethyl |
| G2-2 | 1311690-79-5 | 6-methylpyridin-3-yl | H | (azepan-1-yl)-2- |
| | | | | oxoethyl |
| G2-3 | 1301090-70-9 | 5-chloro-6-(2,2,2- | H | pyridin-2- |
| | | trifluoroethoxy)pyridin-3-yl | | ylmethyl |
| G2-4 | 1299805-79-0 | 6-methylpyridin-3-yl | H | 4-bromobenzyl |
| G2-5 | 1295730-21-0 | 6-(2,2,2-trifluoroethoxy)pyridin-3-yl | H | pyridin-2- |
| | | | | ylmethyl |
| G2-6 | 1288475-13-7 | 5-bromopyridin-3-yl | H | pyridin-2- |
| | | | | ylmethyl |
| G2-7 | 1287863-15-3 | 5-bromopyridin-3-yl | H | 4-bromobenzyl |
| G2-8 | 1279624-84-8 | 6-(2,2,2-trifluoroethoxy)pyridin-3-yl | H | 4-bromobenzyl |
| G2-9 | 1252459-54-3 | 6-methoxypyridin-3-yl | H | benzyl |
| G2-10 | 1241202-60-7 | pyridin-3-yl | H | benzyl |
| G2-11 | 1240880-06-1 | 5-chloro-6-(2,2,2- | H | pyridin-3- |
| | | trifluoroethoxy)pyridin-3-yl | | ylmethyl |
| G2-12 | 1146924-12-0 | pyridin-3-yl | H | 3-chlorobenzyl |
| G2-13 | 1389504-29-3 | 5-chloro-6-(2,2,2- | trifluoromethyl | H |
| | | trifluoroethoxy)pyridin-3-yl | | |
| G2-14 | 1388561-95-2 | 5-chloro-6-(2,2,2- | H | H |
| | | trifluoroethoxy)pyridin-3-yl | | |
| G2-15 | 1301165-37-6 | 6-(2,2,2-trifluoroethoxy)pyridin-3-yl | piperidin-1-yl | H |
| G2-16 | 1279629-65-0 | 5-chloro-6-isopropoxypyridin-3-yl | methylsulphonyl | H |
| G2-17 | 1258777-77-3 | 5-chloro-6-isopropoxypyridin-3-yl | isopropyl | H |
| G2-18 | 1252429-89-2 | pyridin-3-yl | isopropyl | H |
| G2-19 | 1249755-35-8 | 6-methylpyridin-3-yl | H | H |
| G2-20 | 1248113-40-7 | 5,6-dichloropyridin-3-yl | H | H |
| G2-21 | 1241493-74-2 | 5-chloro-6-isopropoxypyridin-3-yl | trifluoromethyl | H |
| G2-22 | 1210286-20-6 | 6-(trifluoromethyl)pyridin-3-yl | H | H |
| G2-23 | 1209076-53-8 | 6-(2,2,2-trifluoroethoxy)pyridin-3-yl | H | H |
| G2-24 | 1099921-76-2 | 6-methylpyridin-3-yl | trifluoromethyl | H |
| G2-25 | 1099895-46-1 | 1-oxidopyridin-3-yl | trifluoromethyl | H |
| G2-26 | 1099290-03-5 | 5-bromopyridin-3-yl | trifluoromethyl | H |
| G2-27 | 1087922-52-8 | 1-oxidopyridin-3-yl | H | H |
| G2-28 | 1087874-75-6 | 6-chloropyridin-3-yl | H | H |
| G2-29 | 1050833-67-4 | 6-methoxypyridin-3-yl | H | H |
| G2-30 | 946667-19-2 | pyridin-3-yl | amino | H |
| G2-31 | 886126-65-4 | pyridin-3-yl | carboxyl | H |
| G2-32 | 500867-55-0 | pyridin-3-yl | methylsulphanyl | H |
| G2-33 | 448240-88-8 | 5-bromopyridin-3-yl | H | H |
| G2-34 | 21051-18-3 | pyridin-3-yl | H | H |

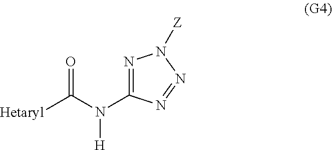

| No. | CAS number | Hetaryl | Z |
|---|---|---|---|
| G4-1 | 925582-52-1 | pyridin-3-yl | vinyl |
| G4-2 | 925573-37-1 | 5-bromopyridin-3-yl | allyl |
| G4-3 | 925167-93-7 | pyridin-3-yl | allyl |
| G4-4 | 925047-93-4 | 5-bromopyridin-3-yl | vinyl |
| G4-5 | 865099-19-0 | pyridin-3-yl | butyl |
| G4-6 | 638147-30-5 | pyridin-3-yl | propyl |
| G4-7 | 585557-66-0 | pyridin-3-yl | methyl |
| G4-8 | 1355508-65-4 | 6-(2,2,3,3-tetrafluoropropoxy) | H |
| | | pyridin-3-yl | |
| G4-9 | 1333542-52-1 | 6-(2,2,2-trifluoroethoxy)pyridin-3-yl | H |
| G4-10 | 1285996-08-8 | 5-bromopyridin-3-yl | H |
| G4-11 | 87884-47-7 | pyridin-3-yl | H |
| G4-12 | 1505086-24-7 | 6-chloropyridin-3-yl | methyl |
| G4-13 | 1500601-31-9 | 6-bromopyridin-3-yl | methyl |
| G4-14 | 1515071-97-2 | 5,6-dichloropyridin-3-yl | methyl |
| G4-15 | 1523210-31-2 | 5-bromopyridin-3-yl | methyl |
| G4-16 | 1533839-68-7 | 6-fluoropyridin-3-yl | methyl |

Modern crop protection compositions, which also include pesticides, have to meet many demands, for example in relation to efficacy, persistence and spectrum of their action and possible use. Questions of toxicity and of combinability with other active ingredients or formulation auxiliaries play a role, as does the question of the expense that the synthesis of an active ingredient requires. In addition, resistances can occur. For all these reasons alone, the search for novel crop protection agents cannot be considered as having been concluded, and there is a constant need for novel compounds having properties which, compared to the known compounds, are improved at least in respect of individual aspects.

It was an object of the present invention to provide compounds which widen the spectrum of the pesticides under various aspects.

This object, and further objects which are not stated explicitly but can be discerned or derived from the connections discussed herein, are achieved by the use of compounds of the formula (I)

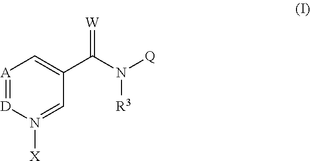

in which

A is N or CR¹,

D is N or CR², where A and D are not both N,

R¹ is a radical from the group of hydrogen, halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl,

R² is a radical from the group of hydrogen, halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl,

X is a free electron pair or oxygen,

W is oxygen, sulphur or NR⁴,

R³ is a radical from the group of hydrogen, cyano, hydroxyl, C₁-C₆-alkyl, C₃-C₆-cycloalkyl, C₁-C₆-haloalkyl, C₃-C₆-halocycloalkyl, C₂-C₆-alkenyl, C₂-C₆-haloalkenyl, C₃-C₆-alkynyl, C₃-C₆-haloalkynyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, C₁-C₆-alkylcarbonyl, C₂-C₆-alkenylcarbonyl, C₃-C₆-alkynylcarbonyl, C₁-C₆-haloalkylcarbonyl, C₃-C₆-cycloalkylcarbonyl, C₃-C₆-cycloalkyl-C₁-C₆-alkylcarbonyl, C₂-C₆-haloalkenylcarbonyl, C₃-C₆-halocycloalkylcarbonyl, C₁-C₆-alkoxy-C₁-C₆-alkylcarbonyl, C₁-C₆-alkoxycarbonyl, C₁-C₆-haloalkoxycarbonyl, C₁-C₆-alkylsulphonyl, C₁-C₆-haloalkylsulphonyl, C₁-C₆-alkylaminocarbonyl, di(C₁-C₆)alkylaminocarbonyl, C₁-C₆-alkylcarbonyloxy, C₁-C₆-alkoxycarbonyloxy, aryl, hetaryl, arylcarbonyl, hetarylcarbonyl, aryl-C₁-C₆-alkyl, hetaryl-C₁-C₆-alkyl, aryl-C₁-C₆-alkylcarbonyl, hetaryl-C₁-C₆-alkylcarbonyl, where the aryl, hetaryl, aryl-C₁-C₆-alkyl, hetaryl-C₁-C₆-alkyl, arylcarbonyl, hetarylcarbonyl, aryl-C₁-C₆-alkylcarbonyl, hetaryl-C₁-C₆-alkylcarbonyl substituents are optionally mono- or polysubstituted, identically or differently, by halogen, cyano, nitro, hydroxyl, C₁-C₆-alkyl, C₂-C₆-alkenyl, C₃-C₆-alkynyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl,

R⁴ is a radical from the group of hydrogen, cyano, nitro, hydroxyl, C₁-C₆-alkyl, C₃-C₆-cycloalkyl, C₁-C₆-haloalkyl, C₃-C₆-halocycloalkyl, C₂-C₆-alkenyl, C₂-C₆-haloalkenyl, C₃-C₆-alkynyl, C₃-C₆-haloalkynyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, C₁-C₆-alkylcarbonyl, C₁-C₆-haloalkylcarbonyl, C₁-C₆-alkoxycarbonyl, C₁-C₆-alkylcarbonyloxy, C₁-C₆-alkylsulphonyl, C₁-C₆-haloalkylsulphonyl, aryl, hetaryl, aryl-C₁-C₆-alkyl, hetaryl-C₁-C₆-alkyl, arylcarbonyl, hetarylcarbonyl, where the aryl, hetaryl, aryl-C₁-C₆-alkyl, hetaryl-C₁-C₆-alkyl, arylcarbonyl, hetarylcarbonyl substituents are optionally mono- or polysubstituted, identically or differently, by halogen, cyano, nitro, hydroxyl, C₁-C₆-alkyl, C₂-C₆-alkenyl, C₃-C₆-alkynyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl,

Q is one of the Q-1 to Q-4 radicals:

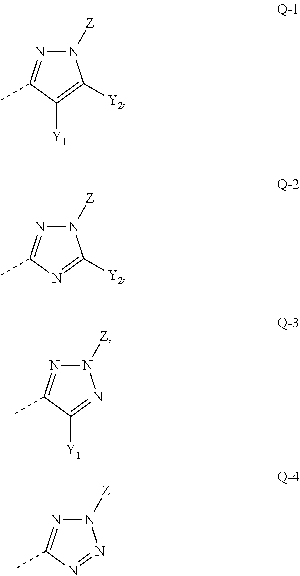

in which the dotted line is the bond to the nitrogen in the NR³ group,

Y₁ is a radical from the group of hydrogen, halogen, cyano, nitro, amino, C₁-C₆-alkylamino, di(C₁-C₆)alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, SH, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di(C₁-C₆)alkylaminocarbonyl,

Y₂ is a radical from the group of hydrogen, halogen, cyano, nitro, amino, C₁-C₆-alkylamino, di(C₁-C₆)alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, SH, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di(C₁-C₆)alkylaminocarbonyl,

in the case that Q=Q-1, Y₁ and Y₂ together with the carbon atoms to which they are bonded form a phenyl ring or a 5- to 7-membered heteroaromatic ring which is optionally substituted by one or more identical or different substituents M¹,

M¹ is halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl and C₁-C₆-alkoxy-C₁-C₆-alkyl,

Z is a radical from the group of hydrogen, cyano, nitro, L¹(=G)C*, L¹O(=G)C*, L³L⁴N(=G)C*, L⁵(=E)CL²N(=G)C*, L⁵(═O)ₙSL²N(=G)C*, L⁵O(=E)CL²N(=G)C*, L¹L⁵N(=E)CL²N(=G)C*, L¹(═O)ₙS*, L³L⁴N(═O)ₙS*, heterocyclyl, aryl, hetaryl, optionally singly or multiply, identically or differently M²-substituted C₃-C₈-cycloalkyl, C₂-C₈-alkenyl, C₅-C₈-cycloalkenyl, C₃-C₈-alkynyl, optionally singly or multiply, identically or differently M⁶-substituted (in the —C₁-C₈-alkyl group) L⁵C(=E)L²N(=G)C—C₁-C₈-alkyl*, L⁵(═O)ₙSL²N(=G)C—C₁-C₈-alkyl*, L⁵OC(=E)L²N(=G)C—C₁-C₈-alkyl*, L¹L⁵NC(=E)L²N(=G)C—C₁-C₈-alkyl*, L¹(═O)ₙS—C₁-C₈-alkyl*, L³O(═O)ₙS—C₁-C₈-alkyl*, L³L⁴N(═O)ₙ—S—C₁-C₈-alkyl*,

M² is halogen, cyano, nitro, NL⁹L¹⁰, OL⁹, SL⁹, L¹(=G)C*, L³O(=G)C*, L³L⁴N(=G)C*, heterocyclyl, aryl, hetaryl, optionally singly or multiply, identically or differently, M³-substituted C₁-C₆-alkyl, C₃-C₆-cycloalkyl, C₂-C₆-alkenyl, C₅-C₆-cycloalkenyl, C₃-C₆-alkynyl, C₁-C₆-alkoxy,

M³ is halogen, cyano, nitro, heterocyclyl, aryl, hetaryl, NL⁹L¹⁰, OL⁹, SL⁹, C₁-C₆-alkyl, C₁-C₆-haloalkyl, C₃-C₆-cycloalkyl, C₃-C₆-halocycloalkyl, C₂-C₆-alkenyl, C₃-C₆-alkynyl, C₁-C₆-alkoxy, C₁-C₆-haloalkoxy, L¹(=G)C*, L³O(=G)C*, L³L⁴N(=G)C*,

G and E are each independently oxygen or sulphur,

n is 1 or 2,

L¹ and L⁵ are each independently heterocyclyl, aryl, hetaryl, optionally singly or multiply, identically or differently, M⁴-substituted C₁-C₆-alkyl, C₃-C₆-cycloalkyl, C₃-C₆-cycloalkyl-C₁-C₆-alkyl, C₂-C₆-alkenyl, C₃-C₆-alkynyl,

M⁴ is halogen, cyano, nitro, heterocyclyl, aryl, hetaryl, C₁-C₆-haloalkyl, C₁-C₆-alkoxy, C₁-C₆-haloalkoxy, NL⁹L¹⁰, OL⁹, SL⁹,

L² is hydrogen, aryl, hetaryl, optionally singly or multiply, identically or differently, M⁵-substituted C₁-C₆-alkyl, C₃-C₆-cycloalkyl, C₃-C₆-cycloalkyl-C₁-C₆-alkyl, C₂-C₆-alkenyl, C₃-C₆-alkynyl,

L³ and L⁴ are each independently hydrogen, heterocyclyl, aryl, hetaryl, optionally singly or multiply, identically or differently, M⁵-substituted C₁-C₆-alkyl, C₃-C₆-cycloalkyl, C₃-C₆-cycloalkyl-C₁-C₆-alkyl, C₂-C₆-alkenyl, C₃-C₆-alkynyl,

M⁵ is halogen, cyano, nitro, heterocyclyl, aryl, hetaryl, C₁-C₆-haloalkyl, C₁-C₆-alkoxy, C₁-C₆-haloalkoxy, C₁-C₆-alkoxycarbonyl, NL⁹L¹⁰, OL⁹, SL⁹,

M⁶ is halogen, cyano, nitro, NL⁹L¹⁰, OL⁹, SL⁹, L¹(=G)C*, L³O(=G)C*, L³L⁴N(=G)C*, heterocyclyl, aryl, hetaryl, C₁-C₆-alkyl, C₁-C₆-haloalkyl, C₃-C₆-cycloalkyl, C₃-C₆-halocycloalkyl, C₃-C₆-cycloalkyl-C₁-C₆-alkyl, C₃-C₆-halocycloalkyl-C₁-C₆-alkyl, C₂-C₆-alkenyl, C₅-C₆-cycloalkenyl, C₃-C₆-alkynyl, C₁-C₆-alkoxy-C₁-C₆-alkyl, C₁-C₆-haloalkoxy-C₁-C₆-alkyl,

L⁹ and L¹⁰ are each independently hydrogen, C₁-C₆-alkyl, C₁-C₆-haloalkyl, C₃-C₆-cycloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₂-C₆-alkenyl, C₂-C₆-haloalkenyl, C₃-C₆-alkynyl, C₁-C₆-alkoxy-C₁-C₆-alkyl, C₁-C₆-haloalkoxy-C₁-C₆-alkyl, C₁-C₆-alkylcarbonyl, C₂-C₆-alkenylcarbonyl, C₃-C₆-alkynylcarbonyl, C₁-C₆-haloalkylcarbonyl, C₃-C₆-cycloalkylcarbonyl, C₃-C₆-cycloalkyl-C₁-C₆-alkylcarbonyl, C₂-C₆-haloalkenylcarbonyl, C₃-C₆-halocycloalkylcarbonyl, C₁-C₆-alkoxycarbonyl, C₁-C₆-alkoxy-C₁-C₆-alkylcarbonyl, C₁-C₆-haloalkoxycarbonyl, C₃-C₆-cycloalkoxycarbonyl, aminocarbonyl, C₁-C₆-alkylaminocarbonyl, di(C₁-C₆)alkylaminocarbonyl, C₃-C₆-cycloalkylaminocarbonyl, C₃-C₆-cycloalkyl-C₁-C₆-alkylaminocarbonyl, aryl, hetaryl, heterocyclyl, aryl-C₁-C₆-alkyl, hetaryl-C₁-C₆-alkyl,

and of the salts and N-oxides thereof for control of animal pests.

In the substituents or ranges mentioned, unless stated otherwise, the heterocyclyl, aryl and hetaryl radicals (including as part of a larger unit such as aryloxy, hetarylalkyl etc.) are optionally mono- or polysubstituted, identically or differently, by halogen, cyano, nitro, SH, hydroxyl, C₁-C₆-alkyl, C₂-C₆-alkenyl, C₃-C₆-alkynyl, C₃-C₆-cycloalkyl, C₁-C₆-haloalkyl, cyano-C₁-C₆-alkyl, C₂-C₆-haloalkenyl, C₃-C₆-haloalkynyl, C₁-C₆-alkoxy, C₂-C₆-alkenyloxy, C₃-C₆-alkynyloxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₃-C₆-cycloalkyl-C₁-C₆-alkyl, C₃-C₆-halocycloalkyl-C₁-C₆-alkyl, amino, C₁-C₆-alkylamino, di(C₁-C₆)alkylamino, C₃-C₆-cycloalkylamino, C₃-C₆-cycloalkyl-C₁-C₆-alkylamino, C₁-C₆-alkylcarbonylamino, C₁-C₆-haloalkylcarbonylamino, C₁-C₆-cycloalkylcarbonylamino, CO₂H, SO₃H, CONH₂, SO₂NH₂, C₁-C₆-alkylcarbonyl, C₂-C₆-alkenylcarbonyl, C₃-C₆-alkynylcarbonyl, C₁-C₆-haloalkylcarbonyl, C₃-C₆-cycloalkylcarbonyl, C₃-C₆-cycloalkyl-C₁-C₆-alkylcarbonyl, C₂-C₆-haloalkenylcarbonyl, C₃-C₆-halocycloalkylcarbonyl, C₁-C₆-alkoxycarbonyl, C₁-C₆-alkoxy-C₁-C₆-alkylcarbonyl, C₁-C₆-haloalkoxycarbonyl, C₃-C₆-cycloalkoxycarbonyl, C₁-C₆-alkylthio, C₁-C₆-haloalkylthio, C₁-C₆-alkylsulphinyl, C₁-C₆-haloalkylsulphinyl, C₁-C₆-alkylsulphonyl, C₁-C₆-haloalkylsulphonyl, C₁-C₆-alkylaminosulphonyl, C₁-C₆-haloalkylaminosulphonyl, di(C₁-C₆)alkylaminosulphonyl, C₁-C₆-alkylaminocarbonyl, di(C₁-C₆)alkylaminocarbonyl, C₁-C₆-alkylcarbonyloxy, C₁-C₆-haloalkylcarbonyloxy, C₁-C₆-alkoxycarbonyloxy, C₁-C₆-haloalkoxycarbonyloxy, aryl, hetaryl, aryloxy, hetaryloxy.

The asterisk (*) in the radical definitions marks the site by which the respective radical is bonded to the remainder of the compound of the formula (I).

By way of example, the corresponding structures are shown for the radicals of the formulae L¹(=G)C, L¹O(=G)C, L³L⁴N(=G)C, L⁵(=E)CL²N(=G)C (corresponding to L⁵C(=E)L²N(=G)C), L⁵(═O)ₙSL²N(=G)C, L⁵O(=E)CL²N(=G)C (corresponding to L⁵OC(=E)L²N(=G)C), L¹L⁵N(=E)CL²N(=G)C), (corresponding to L¹L⁵NC(=E)L²N(=G)C), L¹(═O)ₙS, L³L⁴N(═O)ₙS and L³O(═O)ₙS:

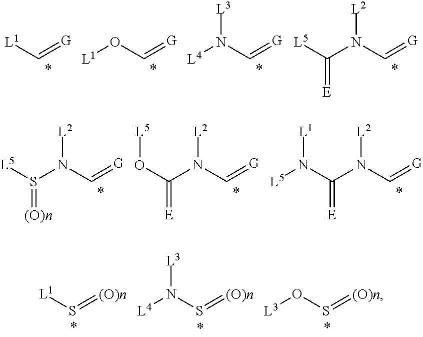

in which the asterisk (*) in each case identifies the atom through which the respective radical is bonded to the remainder of the molecule.

The compounds of the formula (I) may, where appropriate, depending on the nature of the substituents, also be in the form of tautomers and/or stereoisomers, i.e. as geometric isomers and/or as optical isomers or corresponding isomer mixtures in varying composition. The invention relates both to the use of the pure isomers and to that of the isomer mixtures, even though generally only compounds of the formula (I) are discussed here.

Suitable salts of the compounds of the general formula (I) which may be mentioned are customary nontoxic salts, i.e. salts with appropriate bases and salts with added acids. Preference is given to salts with inorganic bases, such as alkali metal salts, for example sodium, potassium or caesium salts, alkaline earth metal salts, for example calcium or magnesium salts, ammonium salts, salts with organic bases and with organic amines, for example triethylammonium, dicyclohexylammonium, N,N′-dibenzylethylenediammonium, pyridinium, picolinium or ethanolammonium salts, salts with inorganic acids, for example hydrochlorides, hydrobromides, sulphates, or phosphates, salts with organic carboxylic acids or organic sulphonic acids, for example formates, acetates, trifluoroacetates, maleates, tartrates, methanesulphonates, benzenesulphonates or para-toluenesulphonates, salts with amino acids, for example arginates, aspartates or glutamates and the like.

The inventive compounds may also be present as metal complexes, as described for other amides, for example, in DE 2221647 A.

Preferred substituents or ranges for the radicals shown in the compounds of the formula (I) are elucidated below. The combination thereof forms the range of preference (1).

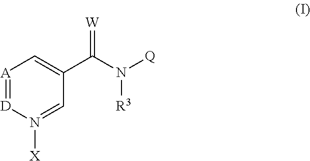

A is N or CR¹.

D is N or CR², where A and D are not both N.

R¹ is a radical from the group of hydrogen, halogen, cyano, nitro, C₁-C₃-alkyl, C₁-C₃-haloalkyl, C₁-C₃-alkoxy, C₁-C₃-haloalkoxy.

R² is a radical from the group of hydrogen, halogen, cyano, nitro, C₁-C₃-alkyl, C₁-C₃-haloalkyl, C₁-C₃-alkoxy, C₁-C₃-haloalkoxy.

X is a free electron pair or oxygen.

W is oxygen, sulphur or NR⁴.

R³ is a radical from the group of hydrogen, cyano, C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, C₁-C₆-alkylcarbonyl, C₁-C₆-haloalkylcarbonyl, C₃-C₆-cycloalkylcarbonyl, C₃-C₆-cycloalkyl-C₁-C₂-alkylcarbonyl, C₃-C₆-halocycloalkylcarbonyl, C₁-C₆-alkoxy-C₁-C₆-alkylcarbonyl, C₁-C₆-alkoxycarbonyl, C₁-C₆-haloalkoxycarbonyl, C₁-C₃-alkylsulphonyl, C₁-C₃-haloalkylsulphonyl, C₁-C₃-alkylaminocarbonyl, di(C₁-C₃)alkylaminocarbonyl, aryl, hetaryl, arylcarbonyl, hetarylcarbonyl, aryl-C₁-C₆-alkyl, hetaryl-C₁-C₆-alkyl, aryl-C₁-C₆-alkylcarbonyl, hetaryl-C₁-C₆-alkylcarbonyl, where the aryl, hetaryl, aryl-C₁-C₆-alkyl, hetaryl-C₁-C₆-alkyl, arylcarbonyl, hetarylcarbonyl, aryl-C₁-C₆-alkylcarbonyl, hetaryl-C₁-C₆-alkylcarbonyl substituents are optionally singly or multiply substituted, identically or differently, by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl.

R⁴ is a radical from the group of hydrogen, cyano, nitro, C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, C₁-C₆-alkylcarbonyl, C₁-C₆-haloalkylcarbonyl, C₁-C₆-alkoxycarbonyl, C₁-C₆-alkylcarbonyloxy, C₁-C₃-alkylsulphonyl, C₁-C₃-haloalkylsulphonyl, aryl, hetaryl, aryl-C₁-C₆ alkyl, hetaryl-C₁-C₆-alkyl, arylcarbonyl, hetarylcarbonyl, where the aryl, hetaryl, aryl-C₁-C₆ alkyl, hetaryl-C₁-C₆-alkyl, arylcarbonyl, hetarylcarbonyl substituents are optionally singly or multiply substituted, identically or differently, by halogen, cyano, nitro, C₁-C₄-alkyl, C₁-C₄-haloalkyl, C₁-C₃-alkoxy, C₁-C₃-haloalkoxy, C₁-C₆-alkoxy-C₁-C₂-alkyl.

Q is one of the Q-1 to Q-4 radicals:

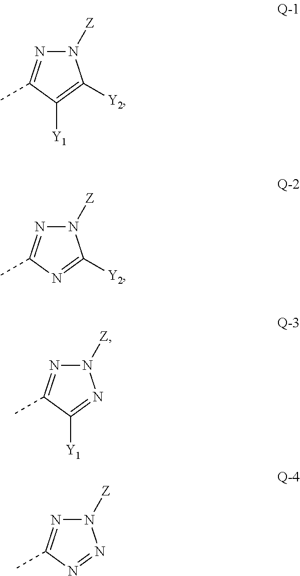

in which the dotted line is the bond to the nitrogen in the NR³ group.

Y₁ is a radical from the group of hydrogen, halogen, cyano, amino, C₁-C₃-alkylamino, di(C₁-C₃)alkylamino, hydroxyl, COOH, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₂-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, SH, C₁-C₆-alkylthio, C₁-C₆-alkylcarbonyl, C₃-C₆-cycloalkylcarbonyl, C₂-C₄-alkenylcarbonyl, C₁-C₆-haloalkylcarbonyl, C₁-C₃-alkoxycarbonyl, C₁-C₆-alkylsulphonyl, C₁-C₃-haloalkylsulphonyl, C₁-C₃-alkylaminocarbonyl, di(C₁-C₃)alkylaminocarbonyl.

Y₂ is a radical from the group of hydrogen, halogen, cyano, amino, C₁-C₃-alkylamino, di(C₁-C₃)alkylamino, hydroxyl, COOH, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₂-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₂-alkyl, SH, C₁-C₆-alkylthio, C₁-C₆-alkylcarbonyl, C₃-C₆-cycloalkylcarbonyl, C₂-C₄-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₃-alkylaminocarbonyl, di(C₁-C₃)alkylaminocarbonyl, or

in the case that Q=Q-1, Y₁ and Y₂ together with the carbon atoms to which they are bonded form a phenyl ring or a 5- to 7-membered heteroaromatic ring which is optionally substituted by one or more identical or different substituents

M¹ is halogen, cyano, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl and C₁-C₆-alkoxy-C₁-C₂-alkyl.

Z is a radical from the group of hydrogen, cyano, L¹(=G)C*, L¹O(=G)C*, L³L⁴N(=G)C*, L⁵(=E)CL²N(=G)C*, L⁵(═O)ₙSL²N(=G)C*, L⁵O(=E)CL²N(=G)C*, L¹L⁵N(=E)CL²N(=G)C*, L¹(═O)ₙS*, L³L⁴N(═O)ₙS*, heterocyclyl, aryl, hetaryl, optionally singly or multiply, identically or differently M²-substituted C₁-C₆-alkyl, C₃-C₆-cycloalkyl, C₂-C₆-alkenyl, C₃-C₆-alkynyl, optionally singly or multiply, identically or differently M⁶-substituted (in the —C₁-C₆-alkyl group) L⁵C(=E)L²N(=G)C—C₁-C₆-alkyl*, L⁵(═O)ₙSL²N(=G)C—C₁-C₆-alkyl*, L⁵OC(=E)L²N(=G)C—C₁-C₆-alkyl*, L¹L⁵NC(=E)L²N(=G)C—C₁-C₆-alkyl*, L¹(═O)ₙS—C₁-C₆-alkyl*, L³O(═O)ₙS—C₁-C₆-alkyl*, L³L⁴N(═O)ₙ—S—C₁-C₆-alkyl*.

M² is halogen, cyano, nitro, NL⁹L¹⁰, OL⁹, SL⁹, L¹(=G)C*, L³O(=G)C*, L³L⁴N(=G)C*, heterocyclyl, aryl, hetaryl, optionally singly or multiply, identically or differently, M³-substituted C₁-C₆-alkyl, C₃-C₆-cycloalkyl, C₂-C₆-alkenyl, C₃-C₆-alkynyl, C₁-C₆-alkoxy.

M³ is halogen, cyano, nitro, heterocyclyl, aryl, hetaryl, NL⁹L¹⁰, OL⁹, SL⁹, C₁-C₆-alkyl, C₁-C₆-haloalkyl, C₃-C₆-cycloalkyl, C₃-C₆-halocycloalkyl, C₂-C₆-alkenyl, C₃-C₆-alkynyl, C₁-C₆-alkoxy, C₁-C₆-haloalkoxy, L¹(=G)C*, L³O(=G)C*, L³L⁴N(=G)C*.

G and E are each independently oxygen or sulphur.

n is 1 or 2.

L¹ and L⁵ are each independently heterocyclyl, aryl, hetaryl, optionally singly or multiply, identically or differently, M⁴-substituted C₁-C₆-alkyl, C₃-C₆-cycloalkyl, C₃-C₆-cycloalkyl-C₁-C₆-alkyl, C₂-C₆-alkenyl, C₃-C₆-alkynyl.

M⁴ is halogen, cyano, nitro, heterocyclyl, aryl, hetaryl, C₁-C₆-haloalkyl, C₁-C₆-alkoxy, C₁-C₆-haloalkoxy, NL⁹L¹⁰, OL⁹, SL⁹.

L² is hydrogen, aryl, hetaryl, optionally singly or multiply, identically or differently, M⁵-substituted C₁-C₆-alkyl, C₃-C₆-cycloalkyl, C₃-C₆-cycloalkyl-C₁-C₆-alkyl, C₂-C₆-alkenyl, C₃-C₆-alkynyl.

L³ and L⁴ are each independently hydrogen, heterocyclyl, aryl, hetaryl, optionally singly or multiply, identically or differently, M⁵-substituted C₁-C₆-alkyl, C₃-C₆-cycloalkyl, C₃-C₆-cycloalkyl-C₁-C₆-alkyl, C₂-C₆-alkenyl, C₃-C₆-alkynyl.

M⁵ is halogen, cyano, nitro, heterocyclyl, aryl, hetaryl, C₁-C₆-haloalkyl, C₁-C₆-alkoxy, C₁-C₆-haloalkoxy, C₁-C₆-alkoxycarbonyl, NL⁹L¹⁰, OL⁹, SL⁹.

M⁶ is halogen, cyano, nitro, NL⁹L¹⁰, OL⁹, SL⁹, L¹(=G)C*, L³O(=G)C*, L³L⁴N(=G)C*, heterocyclyl, aryl, hetaryl, C₁-C₆-alkyl, C₁-C₆-haloalkyl, C₃-C₆-cycloalkyl, C₃-C₆-halocycloalkyl, C₃-C₆-cycloalkyl-C₁-C₆-alkyl, C₃-C₆-halocycloalkyl-C₁-C₆-alkyl, C₂-C₆-alkenyl, C₃-C₆-alkynyl, C₁-C₆-alkoxy-C₁-C₃-alkyl, C₁-C₆-haloalkoxy-C₁-C₃-alkyl.

L⁹ and L¹⁰ are each independently hydrogen, C₁-C₆-alkyl, C₁-C₆-haloalkyl, C₃-C₆-cycloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₂-C₄-alkenyl, C₂-C₄-haloalkenyl, C₃-C₆-alkynyl, C₁-C₆-alkoxy-C₁-C₆-alkyl, C₁-C₆-haloalkoxy-C₁-C₆-alkyl, C₁-C₆-alkylcarbonyl, C₂-C₄-alkenylcarbonyl, C₃-C₆-alkynylcarbonyl, C₁-C₆-haloalkylcarbonyl, C₃-C₆-cycloalkylcarbonyl, C₃-C₆-cycloalkyl-C₁-C₆-alkylcarbonyl, C₂-C₄-haloalkenylcarbonyl, C₃-C₆-halocycloalkylcarbonyl, C₁-C₆-alkoxycarbonyl, C₁-C₆-alkoxy-C₁-C₆-alkylcarbonyl, C₁-C₆-haloalkoxycarbonyl, C₃-C₆-cycloalkoxycarbonyl, aminocarbonyl, C₁-C₆-alkylaminocarbonyl, di(C₁-C₆)alkylaminocarbonyl, C₃-C₆-cycloalkylaminocarbonyl, C₃-C₆-cycloalkyl-C₁-C₆-alkylaminocarbonyl, aryl, hetaryl, heterocyclyl, aryl-C₁-C₆-alkyl, hetaryl-C₁-C₆-alkyl.

In the preferred substituents or ranges mentioned, unless stated otherwise, the heterocyclyl, aryl and hetaryl radicals (including as part of a larger unit such as aryloxy, hetarylalkyl etc.) are optionally mono- or polysubstituted, identically or differently, by halogen, cyano, nitro, SH, hydroxyl, C₁-C₆-alkyl, C₃-C₆-cycloalkyl, C₁-C₆-haloalkyl, cyano-C₁-C₆-alkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₃-C₆-cycloalkyl-C₁-C₆-alkyl, C₃-C₆-halocycloalkyl-C₁-C₆-alkyl, amino, C₁-C₆-alkylamino, di(C₁-C₆)alkylamino, C₃-C₆-cycloalkylamino, C₃-C₆-cycloalkyl-C₁-C₆-alkylamino, C₁-C₆-alkylcarbonylamino, C₁-C₆-haloalkylcarbonylamino, C₁-C₆-cycloalkylcarbonylamino, CO₂H, SO₃H, CONH₂, SO₂NH₂, C₁-C₆-alkylcarbonyl, C₂-C₆-alkenylcarbonyl, C₃-C₆-alkynylcarbonyl, C₁-C₆-haloalkylcarbonyl, C₃-C₆-cycloalkylcarbonyl, C₃-C₆-cycloalkyl-C₁-C₆-alkylcarbonyl, C₂-C₆-haloalkenylcarbonyl, C₃-C₆-halocycloalkylcarbonyl, C₁-C₆-alkoxycarbonyl, C₁-C₆-alkoxy-C₁-C₆-alkylcarbonyl, C₁-C₆-haloalkoxycarbonyl, C₃-C₆-cycloalkoxycarbonyl, C₁-C₆-alkylthio, C₁-C₆-haloalkylthio, C₁-C₆-alkylsulphinyl, C₁-C₆-haloalkylsulphinyl, C₁-C₆-alkylsulphonyl, C₁-C₆-haloalkylsulphonyl, C₁-C₆-alkylaminosulphonyl, C₁-C₆-haloalkylaminosulphonyl, di(C₁-C₆)alkylaminosulphonyl, C₁-C₆-alkylaminocarbonyl, di(C₁-C₆)alkylaminocarbonyl, C₁-C₆-alkylcarbonyloxy, C₁-C₆-haloalkylcarbonyloxy, C₁-C₆-alkoxycarbonyloxy, C₁-C₆-haloalkoxycarbonyloxy, aryl, hetaryl, aryloxy, hetaryloxy.

Particularly preferred substituents or ranges for the radicals shown in the compounds of the formula (I) are elucidated below. The combination thereof forms the range of preference (2).

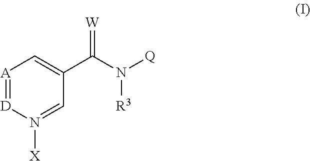

A is N or CR¹.

D is N or CR², where A and D are not both N.

R¹ is a radical from the group of hydrogen, halogen, cyano, methyl, halomethyl, methoxy, halomethoxy.

R² is a radical from the group of hydrogen, halogen, cyano, methyl, halomethyl, methoxy, halomethoxy.

X is a free electron pair or oxygen.

W is oxygen, sulphur or NR⁴.

R³ is a radical from the group of hydrogen, C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkylcarbonyl, C₁-C₆-haloalkylcarbonyl, C₃-C₆-cycloalkylcarbonyl, C₁-C₆-alkoxy-C₁-C₆-alkylcarbonyl, C₁-C₆-alkoxycarbonyl, C₁-C₆-haloalkoxycarbonyl, aryl, hetaryl, arylcarbonyl, hetarylcarbonyl, where the aryl, hetaryl, arylcarbonyl and hetarylcarbonyl substituents are optionally singly or multiply substituted, identically or differently, by halogen, cyano, C₁-C₃-alkyl, C₁-C₃-haloalkyl, C₁-C₃-alkoxy, C₁-C₃-haloalkoxy, C₁-C₃-alkoxy-C₁-C₂-alkyl.

R⁴ is a radical from the group of hydrogen, cyano, nitro, C₁-C₃-alkyl, C₃-C₆-cycloalkyl, C₁-C₃-haloalkyl, C₃-C₆-halocycloalkyl, C₃-C₆-cycloalkyl-C₁-C₃-alkyl, C₃-C₆-halocycloalkyl-C₁-C₃-alkyl, cyano-C₁-C₃-alkyl, C₁-C₃-alkoxy, C₁-C₃-haloalkoxy, C₁-C₃-alkoxy-C₁-C₃-alkyl, C₁-C₃-haloalkoxy-C₁-C₃-alkyl, C₁-C₄-alkylcarbonyl, C₁-C₄-haloalkylcarbonyl, C₁-C₃-alkoxycarbonyl, C₁-C₃-alkylcarbonyloxy.

Q is one of the Q-1 to Q-3 radicals:

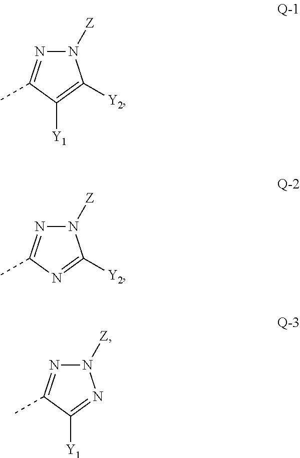

in which the dotted line is the bond to the nitrogen in the NR³ group.

Y₁ is a radical from the group of hydrogen, halogen, cyano, hydroxyl, C₁-C₆-alkyl, C₃-C₆-cycloalkyl, C₁-C₆-haloalkyl, C₁-C₆-alkoxy, C₁-C₆-haloalkoxy, C₁-C₆-alkoxy-C₁-C₃-alkyl, C₁-C₆-haloalkoxy-C₁-C₃-alkyl, C₁-C₆-alkoxy-C₁-C₃-alkyloxy, cyano-C₁-C₃-alkyl, C₃-C₆-cycloalkyl-C₁-C₃-alkyl, SH, C₁-C₃-alkylthio, C₁-C₄-alkylcarbonyl, C₁-C₄-haloalkylcarbonyl, C₁-C₃-alkylsulphonyl.

Y₂ is a radical from the group of hydrogen, halogen, cyano, hydroxyl, C₁-C₆-alkyl, C₃-C₆-cycloalkyl, C₁-C₆-haloalkyl, C₁-C₆-alkoxy, C₁-C₆-haloalkoxy, C₁-C₆-alkoxy-C₁-C₄-alkyl, C₁-C₆-haloalkoxy-C₁-C₄-alkyl, C₁-C₆-alkoxy-C₁-C₄-alkyloxy, cyano-C₁-C₃-alkyl, C₃-C₆-cycloalkyl-C₁-C₂-alkyl, SH, C₁-C₃-alkylthio, C₁-C₄-alkylcarbonyl, C₁-C₄-haloalkylcarbonyl, C₁-C₄-alkoxycarbonyl, C₁-C₃-alkylsulphonyl, C₁-C₃-haloalkylsulphonyl, or

in the case that Q=Q-1, Y₁ and Y₂ together with the carbon atoms to which they are bonded form a phenyl ring or a 5- to 7-membered heteroaromatic ring which is optionally substituted by one or more identical or different substituents

M¹ is halogen, cyano, C₁-C₄ alkyl, C₁-C₄-haloalkyl, C₁-C₄-alkoxy and C₁-C₄-haloalkoxy.

Z is a radical from the group of hydrogen, L¹(=G)C*, L¹O(=G)C*, L³L⁴N(=G)C*, L⁵(=E)CL²N(=G)C*, L⁵(═O)ₙSL²N(=G)C*, L⁵O(=E)CL²N(=G)C*, L¹L⁵N(=E)CL²N(=G)C*, L¹(═O)ₙS*, L³L⁴N(═O)ₙS*, heterocyclyl, aryl, hetaryl, optionally singly or multiply, identically or differently M²-substituted C₁-C₆-alkyl, C₃-C₆-cycloalkyl, C₂-C₆-alkenyl, C₃-C₆-alkynyl, optionally singly or multiply, identically or differently M⁶-substituted (in the —C₁-C₄-alkyl group) L⁵C(=E)L²N(=G)C—C₁-C₄-alkyl*, L⁵(═O)ₙSL²N(=G)C—C₁-C₄-alkyl*, L⁵OC(=E)L²N(=G)C—C₁-C₄-alkyl*, L¹L⁵NC(=E)L²N(=G)C—C₁-C₄-alkyl*, L¹(═O)ₙS—C₁-C₄-alkyl*, L³O(═O)ₙS—C₁-C₄-alkyl*, L³L⁴N(═O)ₙS—C₁-C₄-alkyl*.

M² is halogen, cyano, NL⁹L¹⁰, OL⁹, SL⁹, L¹(=G)C*, L³O(=G)C*, L³L⁴N(=G)C*, heterocyclyl, aryl, hetaryl, C₁-C₄-alkyl, C₁-C₄-haloalkyl, C₁-C₄-cyanoalkyl, C₃-C₆-cycloalkyl, C₃-C₆-halocycloalkyl.

G and E are each independently oxygen or sulphur.

n is 1 or 2.

L¹ and L⁵ are each independently heterocyclyl, aryl, hetaryl, optionally singly or multiply, identically or differently, M⁴-substituted C₁-C₆-alkyl, aryl-C₁-C₄-alkyl, hetaryl-C₁-C₄-alkyl, C₃-C₆-cycloalkyl, C₃-C₆-cycloalkyl-C₁-C₄-alkyl, C₂-C₆-alkenyl, C₃-C₆-alkynyl.

M⁴ is halogen, cyano, C₁-C₃-haloalkyl, C₁-C₃-alkoxy, C₁-C₃-haloalkoxy.

L² is hydrogen, aryl, hetaryl, optionally singly or multiply, identically or differently, M⁵-substituted C₁-C₆-alkyl, aryl-C₁-C₄-alkyl, hetaryl-C₁-C₄-alkyl, C₃-C₆-cycloalkyl, C₃-C₆-cycloalkyl-C₁-C₄-alkyl.

L³ and L⁴ are each independently hydrogen, heterocyclyl, aryl, hetaryl, optionally singly or multiply, identically or differently, M⁵-substituted C₁-C₆-alkyl, aryl-C₁-C₄-alkyl, hetaryl-C₁-C₄-alkyl, C₃-C₆-cycloalkyl, C₃-C₆-cycloalkyl-C₁-C₄-alkyl.

M⁵ is halogen, cyano, C₁-C₆-haloalkyl, C₁-C₆-alkoxy, C₁-C₆-haloalkoxy, C₁-C₄-alkoxycarbonyl.

M⁶ is halogen, cyano, NL⁹L¹⁰, OL⁹, aryl, hetaryl, C₁-C₃-haloalkyl, C₃-C₄-cycloalkyl, C₃-C₄-halocycloalkyl, C₃-C₄-cycloalkyl-C₁-C₃-alkyl, C₃-C₄-halocycloalkyl-C₁-C₃-alkyl.

L⁹ and L¹⁰ are each independently hydrogen, C₁-C₆-alkyl, C₁-C₆-haloalkyl, C₃-C₆-cycloalkyl, C₃-C₆-halocycloalkyl, C₃-C₆-cycloalkyl-C₁-C₄-alkyl, C₃-C₆-halocycloalkyl-C₁-C₄-alkyl, cyano-C₁-C₆-alkyl, C₁-C₆-alkoxy-C₁-C₄-alkyl, C₁-C₆-haloalkoxy-C₁-C₄-alkyl, C₁-C₆-alkylcarbonyl, C₁-C₆-haloalkylcarbonyl, C₃-C₆-cycloalkylcarbonyl, C₃-C₆-cycloalkyl-C₁-C₄-alkylcarbonyl, C₃-C₆-halocycloalkylcarbonyl, C₁-C₆-alkoxycarbonyl, C₁-C₆-alkoxy-C₁-C₄-alkylcarbonyl, C₁-C₆-haloalkoxycarbonyl, C₃-C₆-cycloalkoxycarbonyl, C₁-C₆-alkylaminocarbonyl, di(C₁-C₆)alkylaminocarbonyl, C₃-C₆-cycloalkylaminocarbonyl, C₃-C₆-cycloalkyl-C₁-C₆-alkylaminocarbonyl, aryl, hetaryl, heterocyclyl, aryl-C₁-C₆-alkyl, hetaryl-C₁-C₆-alkyl.

In the particularly preferred substituents or ranges mentioned, unless stated otherwise, the heterocyclyl, aryl and hetaryl radicals (including as part of a larger unit such as aryloxy, hetarylalkyl etc.) are optionally mono- or polysubstituted, identically or differently, by halogen, cyano, C₁-C₆-alkyl, C₃-C₆-cycloalkyl, C₁-C₆-haloalkyl, cyano-C₁-C₄-alkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₁-C₄-alkylcarbonylamino, C₁-C₄-haloalkylcarbonylamino, C₁-C₆-cycloalkylcarbonylamino, C₁-C₄-alkylcarbonyl, C₁-C₄-haloalkylcarbonyl, C₃-C₆-cycloalkylcarbonyl, C₃-C₆-cycloalkyl-C₁-C₂-alkylcarbonyl, C₃-C₆-halocycloalkylcarbonyl, C₁-C₆-alkoxycarbonyl, C₁-C₆-alkoxy-C₁-C₂-alkylcarbonyl, C₁-C₆-haloalkoxycarbonyl, C₁-C₆-alkylthio, C₁-C₆-haloalkylthio, C₁-C₆-alkylsulphinyl, C₁-C₆-haloalkylsulphinyl, C₁-C₆-alkylsulphonyl, C₁-C₆-haloalkylsulphonyl, C₁-C₆-alkylaminosulphonyl, C₁-C₆-haloalkylaminosulphonyl, di(C₁-C₆)alkylaminosulphonyl, C₁-C₆-alkylaminocarbonyl, di(C₁-C₆)alkylaminocarbonyl, C₁-C₆-alkyl-carbonyloxy, C₁-C₄-haloalkylcarbonyloxy, C₁-C₄-alkoxycarbonyloxy, C₁-C₄-haloalkoxycarbonyloxy, aryl, hetaryl, aryloxy, hetaryloxy.

Very particularly preferred substituents or ranges of the radicals shown in the compounds of the formula (I) are elucidated below. The combination thereof forms the range of preference (3).

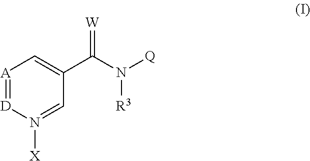

A is CR¹.

D is CR².

R¹ is hydrogen or fluorine.

R² is hydrogen.

X is a free electron pair or oxygen.

W is oxygen or sulphur.

R³ is a radical from the group of hydrogen, C₁-C₄-alkyl, C₃-C₄-cycloalkyl, C₁-C₄-haloalkyl, C₃-C₄-cycloalkyl-C₁-C₂-alkyl, C₁-C₃-alkoxy-C₁-C₂-alkyl, C₁-C₄-alkylcarbonyl, C₁-C₄-haloalkylcarbonyl, C₃-C₄-cycloalkylcarbonyl, C₁-C₃-alkoxycarbonyl, aryl, hetaryl, arylcarbonyl, hetarylcarbonyl, aryl-C₁-C₃-alkyl, hetaryl-C₁-C₃-alkyl, where the aryl, hetaryl, aryl-C₁-C₃-alkyl, hetaryl-C₁-C₃-alkyl, arylcarbonyl and hetarylcarbonyl substituents are optionally mono- or polysubstituted by halogen.

Q is Q-1

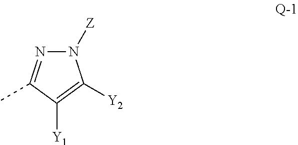

in which the dotted line is the bond to the nitrogen in the NR³ group.

Y₁ is a radical from the group of hydrogen, halogen, cyano, C₁-C₄ alkyl, C₁-C₄-haloalkyl, C₁-C₃-alkoxy, C₁-C₃-haloalkoxy.

Y₂ is a radical from the group of hydrogen, halogen, cyano, C₃-C₅-cycloalkyl, C₁-C₄-haloalkyl, C₁-C₄-alkoxy, C₁-C₄-haloalkoxy, C₁-C₃-alkoxy-C₁-C₄-alkyl, C₁-C₃-haloalkoxy-C₁-C₄-alkyl, C₁-C₃-alkoxy-C₁-C₄-alkyloxy, C₁-C₄-alkoxycarbonyl, or

in the case that Q=Q-1, Y₁ and Y₂ together with the carbon atoms to which they are bonded form a phenyl ring or a 5- to 7-membered heteroaromatic ring which is optionally substituted by one or more identical or different substituents

M¹ is halogen, cyano, C₁-C₃-alkyl, C₁-C₃-haloalkyl, C₁-C₂-alkoxy and C₁-C₂-haloalkoxy.

Z is a radical from the group of hydrogen, L¹(=G)C*, L¹O(=G)C*, L³L⁴N(=G)C*, L¹(═O)ₙS*, L³L⁴N(═O)ₙS*, heterocyclyl, aryl, hetaryl, singly or multiply, identically or differently, M²-substituted C₁-C₆-alkyl, C₃-C₆-cycloalkyl, C₂-C₆-alkenyl, C₃-C₆-alkynyl, optionally singly or multiply, identically or differently, M⁶-substituted (in the —C₁-C₄-alkyl group) L⁵(═O)ₙSL²N(=G)C—C₁-C₄-alkyl*, L¹(═O)ₙS—C₁-C₄-alkyl*.

M² is halogen, cyano, NL⁹L¹⁰, OL⁹, SL⁹, L¹(=G)C*, L³O(=G)C*, L³L⁴N(=G)C*, heterocyclyl, aryl, hetaryl, C₁-C₄-alkyl, C₁-C₄-haloalkyl, C₁-C₄-cyanoalkyl, C₃-C₄-cycloalkyl, C₃-C₄-halocycloalkyl.

G is oxygen.

n is 1 or 2.

L¹ and L⁵ are each independently heterocyclyl, aryl, hetaryl, optionally singly or multiply, identically or differently, M⁴-substituted C₁-C₄-alkyl, aryl-C₁-C₄-alkyl, hetaryl-C₁-C₄-alkyl, C₃-C₆-cycloalkyl, C₃-C₆-cycloalkyl-C₁-C₄-alkyl.

M⁴ is halogen, cyano, C₁-C₃-haloalkyl, C₁-C₃-alkoxy, C₁-C₃-haloalkoxy.

L² is hydrogen, optionally singly or multiply, identically or differently, M⁵-substituted C₁-C₄-alkyl, C₃-C₆-cycloalkyl, C₃-C₆-cycloalkyl-C₁-C₄-alkyl.

L³ and L⁴ are each independently hydrogen, heterocyclyl, aryl, hetaryl, optionally singly or multiply, identically or differently, M⁵-substituted C₁-C₄-alkyl, aryl-C₁-C₄-alkyl, hetaryl-C₁-C₄-alkyl, C₃-C₆-cycloalkyl, C₃-C₆-cycloalkyl-C₁-C₄-alkyl.

M⁵ is halogen, cyano, C₁-C₃-haloalkyl, C₁-C₃-alkoxy, C₁-C₄-alkoxycarbonyl.

M⁶ is halogen, C₁-C₃-haloalkyl.

L⁹ and L¹⁰ are each independently hydrogen, C₁-C₄-alkyl, C, C₄ haloalkyl, C₃-C₆-cycloalkyl, C₃-C₆-halocycloalkyl, C₃-C₆-cycloalkyl-C₁-C₄-alkyl, C₃-C₆-halocycloalkyl-C₁-C₄-alkyl, cyano-C₁-C₄-alkyl, C₁-C₄-alkoxy-C₁-C₄-alkyl, C₁-C₄-haloalkoxy-C₁-C₄-alkyl, C₁-C₄-alkylcarbonyl, C₁-C₄-haloalkylcarbonyl, C₃-C₆-cycloalkylcarbonyl, C₃-C₆-cycloalkyl-C₁-C₄-alkylcarbonyl, C₃-C₆-halocycloalkylcarbonyl, C₁-C₄-alkoxycarbonyl, C₁-C₄-alkoxy-C₁-C₄-alkylcarbonyl, C₁-C₄-haloalkoxycarbonyl, C₁-C₄-alkylaminocarbonyl, di(C₁-C₄)alkylaminocarbonyl, C₃-C₆-cycloalkylaminocarbonyl, C₃-C₆-cycloalkyl-C₁-C₄-alkylaminocarbonyl, aryl, hetaryl, heterocyclyl, aryl-C₁-C₃-alkyl, hetaryl-C₁-C₃-alkyl.

In the very particularly preferred substituents or ranges mentioned, unless stated otherwise, the heterocyclyl, aryl and hetaryl radicals (including as part of a larger unit such as aryloxy, hetarylalkyl etc.) are optionally mono- or polysubstituted, identically or differently, by halogen, cyano, C, C₄ alkyl, C₃-C₆-cycloalkyl, C, C₄ haloalkyl, C₁-C₄-alkoxy, C₁-C₄-haloalkoxy, C₃-C₆-halocycloalkyl, C₁-C₄-haloalkoxy-C₁-C₄-alkyl, C₁-C₄-alkoxycarbonyl, C₁-C₄-haloalkoxycarbonyl, C₁-C₃-alkylthio, C₁-C₃-haloalkylthio, C₁-C₃-alkylsulphonyl, C₁-C₃-haloalkylsulphonyl, C₁-C₃-alkylaminosulphonyl, C₁-C₃-haloalkylaminosulphonyl, di(C₁-C₃)alkylaminosulphonyl, C₁-C₃-alkylaminocarbonyl, di(C₁-C₃)alkylaminocarbonyl, aryl, hetaryl, aryloxy, hetaryloxy.

Explicitly very particularly preferred substituents or ranges of the radicals shown in the compounds of the formula (I) are elucidated below. The combination thereof forms the range of preference (4).

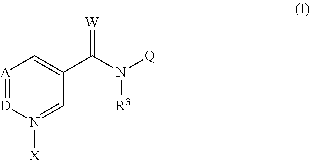

A is CR¹.

D is CR².

R¹ is hydrogen or fluorine.

R² is hydrogen.

X is a free electron pair.

W is oxygen.

R³ is a radical from the group of hydrogen, C₁-C₄-alkyl, C₁-C₄-haloalkyl, C₃-C₄-cycloalkyl-C₁-C₂-alkyl, C₁-C₄-alkylcarbonyl, arylcarbonyl, hetarylcarbonyl, where the arylcarbonyl and hetarylcarbonyl substituents are optionally mono- or polysubstituted by halogen.

Q is Q-1

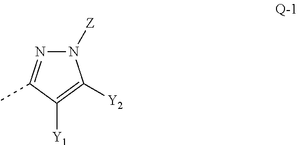

in which the dotted line is the bond to the nitrogen in the NR³ group.

Y₁ is a radical from the group of hydrogen, halogen, cyano, methyl, ethyl, n-propyl, iso-propyl, trifluoromethyl, difluoromethyl, methoxy, ethoxy, difluoromethoxy, trifluoromethoxy.

Y₂ is a radical from the group of hydrogen, halogen, cyano, C₁-C₄-alkyl, C₃-C₅-cycloalkyl, C₁-C₃-haloalkyl, C₁-C₃-alkoxy, C₁-C₃-haloalkoxy, C₁-C₃-alkoxy-C₁-C₂-alkyl, C₁-C₃-haloalkoxy-C₁-C₂-alkyl, C₁-C₃-alkoxy-C₁-C₂-alkyloxy, C₁-C₃-alkoxycarbonyl.

Z is a radical from the group of hydrogen, L¹(=G)C*, L¹O(=G)C*, L³L⁴N(=G)C*, L¹(═O)ₙS*, L³L⁴N(═O)ₙS*, aryl, hetaryl, C₃-C₆-cycloalkyl, C₃-C₆-halocycloalkyl, C₂-C₆-alkenyl, C₃-C₆-alkynyl, C₂-C₆-haloalkenyl, C₃-C₆-haloalkynyl, optionally singly or multiply, identically or differently, M²-substituted C₁-C₆-alkyl, optionally singly or multiply, identically or differently, M⁶-substituted (in the —C₁-C₄-alkyl group) L⁵(═O)ₙSL²N(=G)C—C₁-C₄-alkyl*,

M² is halogen, cyano, heterocyclyl, aryl, hetaryl, C₃-C₄-cycloalkyl, C₃-C₄-halocycloalkyl, NL⁹L¹⁰, OL⁹, SL⁹, L¹(=G)C*, L³O(=G)C*, L³L⁴N(=G)C*.

G is oxygen.

n is 1 or 2.

L¹ and L⁵ are each independently aryl, hetaryl, C₁-C₄-alkyl, C₁-C₄-haloalkyl, C₁-C₄-cyanoalkyl, C₁-C₃-alkoxy-C₁-C₄-alkyl, C₁-C₃-haloalkoxy-C₁-C₄-alkyl, aryl-C₁-C₄-alkyl, aryl-C₁-C₄-haloalkyl, aryl-C₁-C₄-cyanoalkyl, hetaryl-C₁-C₄-alkyl, hetaryl-C₁-C₄-haloalkyl, hetaryl-C₁-C₄-cyanoalkyl, C₃-C₆-cycloalkyl, C₃-C₆-halocycloalkyl, C₃-C₆-cycloalkyl-C₁-C₄-alkyl, C₃-C₆-halocycloalkyl-C₁-C₄-alkyl.

L² is hydrogen or C₁-C₄-alkyl.

L³ and L⁴ are each independently hydrogen, aryl, hetaryl, C₁-C₄-alkyl, C₁-C₄-haloalkyl, C₁-C₄-cyanoalkyl, C₁-C₃-alkoxy-C₁-C₄-alkyl, aryl-C₁-C₄-alkyl, aryl-C₁-C₄-haloalkyl, aryl-C₁-C₄-cyanoalkyl, hetaryl-C₁-C₄-alkyl, hetaryl-C₁-C₄-haloalkyl, hetaryl-C₁-C₄-cyanoalkyl, C₃-C₆-cycloalkyl, C₃-C₆-halocycloalkyl, C₃-C₆-cycloalkyl-C₁-C₄-alkyl, C₃-C₆-halocycloalkyl-C₁-C₄-alkyl.

M⁶ is halogen, cyano, C₁-C₃-haloalkyl.

L⁹ and L¹⁰ are each independently hydrogen, C₁-C₄-alkyl, C₁-C₄-haloalkyl, C₃-C₆-cycloalkyl, C₃-C₆-cycloalkyl-C₁-C₄-alkyl, cyano-C₁-C₄-alkyl, C₁-C₄-alkylcarbonyl, C₁-C₄-haloalkylcarbonyl, C₃-C₆-cycloalkyl-C₁-C₄-alkylcarbonyl, C₁-C₄-alkoxycarbonyl, C₁-C₄-alkylaminocarbonyl, di(C₁-C₄)alkylaminocarbonyl, C₃-C₆-cycloalkylaminocarbonyl, C₃-C₆-cycloalkyl-C₁-C₄-alkylaminocarbonyl, aryl, hetaryl, aryl-C₁-C₃-alkyl, hetaryl-C₁-C₃-alkyl.

In the explicitly very particularly preferred substituents or ranges mentioned, unless stated otherwise, the heterocyclyl, aryl and hetaryl radicals (including as part of a larger unit such as aryloxy, hetarylalkyl etc.) are optionally mono- or polysubstituted, identically or differently, by halogen, cyano, C₁-C₃-alkyl, C₁-C₃-haloalkyl, C₁-C₂-alkoxy, C₁-C₂-haloalkoxy, C₁-C₃-alkoxycarbonyl, C₁-C₃-haloalkoxycarbonyl, C₁-C₂-alkylthio, C₁-C₂-haloalkylthio, C₁-C₂-alkylsulphonyl, C₁-C₂-haloalkylsulphonyl, C₁-C₃-alkylaminosulphonyl, C₁-C₃-alkylaminocarbonyl, aryl, hetaryl, aryloxy, hetaryloxy.

A preferred embodiment of the invention relates to the use of compounds of the formula (I) in which the substituents or ranges of the radicals listed are defined as follows. The combination thereof forms the range of preference (5).

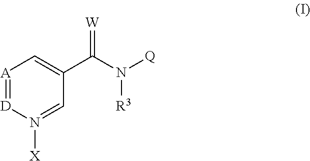

A is N or CR¹.

D is N or CR², where A and D are not both N.

R¹ is hydrogen, chlorine, bromine or fluorine.

R² is hydrogen, methyl, methoxy or chlorine.

X is a free electron pair or oxygen.

W is oxygen or sulphur.

R³ is a radical from the group of hydrogen, C₁-C₃-alkyl, C₁-C₃-haloalkyl, C₃-C₄-alkynyl, cyano-C₁-C₂-alkyl, arylcarbonyl and hetaryl-C₁-C₂-alkyl, where the arylcarbonyl and hetaryl-C₁-C₂-alkyl substituents are optionally singly or multiply substituted, identically or differently, by halogen.

Q is Q-1

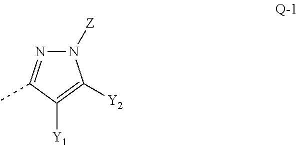

in which the dotted line is the bond to the nitrogen in the NR³ group.

Y₁ is a radical from the group of hydrogen, halogen, C₁-C₃-alkyl, cyano and C₁-C₂-alkoxycarbonyl,

Y₂ is a radical from the group of hydrogen, C₁-C₃-alkyl, C₃-C₅-cycloalkyl, C₁-C₂-haloalkyl, halogen, cyano, C₁-C₂-haloalkoxy, C₁-C₂-alkoxycarbonyl, C₁-C₂-alkylthio and C₁-C₂-alkylsulphinyl, or

Y₁ and Y₂ together with the carbon atoms to which they are bonded form a phenyl ring or a 6-membered heteroaromatic ring which is optionally mono- or polysubstituted by methyl.

Z is a radical from the group of hydrogen, C₁-C₆-alkyl, C₁-C₄-haloalkyl, C₃-C₇-cycloalkyl, C₂-C₆-alkenyl, C₂-C₄-haloalkenyl, C₃-C₅-alkynyl, cyano-C₁-C₄-alkyl, C₃-C₅-cycloalkyl-C₁-C₃-alkyl, C₃-C₅-halocycloalkyl-C₁-C₂-alkyl, aryl, hetaryl, aryl-C₁-C₂-alkyl, hetaryl-C₁-C₂-alkyl, hetaryl-C₁-C₃-haloalkyl, heterocyclyl-C₁-C₂-alkyl, C₁-C₆-alkylaminocarbonyl, C₃-C₆-cycloalkylaminocarbonyl, C₁-C₃-haloalkylaminocarbonyl, C₃-C₅-cycloalkyl-C₁-C₂-alkylaminocarbonyl, C₁-C₂-alkoxy-C₁-C₄-alkylaminocarbonyl, di(C₁-C₃-alkoxy)-C₁-C₄-alkylaminocarbonyl, C₁-C₂-haloalkoxy-C₁-C₃-alkylaminocarbonyl, di(C₁-C₃)alkylaminocarbonyl, di(C₁-C₂-alkoxy-C₁-C₄-alkyl)aminocarbonyl, aryl-C₁-C₄-alkylaminocarbonyl, hetaryl-C₁-C₄-alkylaminocarbonyl, heterocyclyl-C₁-C₃-alkylaminocarbonyl, aryloxy-C₁-C₃-alkylaminocarbonyl, C₁-C₂-alkylsulphonyl-(C₁-C₂-alkylamino)carbonyl, arylsulphonyl-(C₁-C₂-alkylamino)carbonyl, C₁-C₄-alkylaminosulphonyl, di(C₁-C₂)alkylaminosulphonyl, C₁-C₃-alkylsulphonyl, hetarylsulphonyl, heterocyclylsulphonyl, C₁-C₅-alkoxycarbonyl, C₁-C₃-haloalkoxycarbonyl, aryloxycarbonyl, C₁-C₅-alkylcarbonyl, C₃-C₅-cycloalkylcarbonyl, arylcarbonyl, hetarylcarbonyl, heterocyclylcarbonyl, C₁-C₂-alkoxy-C₁-C₃-alkyl, C₁-C₃-alkylthio-C₁-C₃-alkyl, C₁-C₃-alkylsulphinyl-C₁-C₃-alkyl, C₁-C₃-alkylsulphonyl-C₁-C₃-alkyl, hetarylthio-C₁-C₂-alkyl, hetarylsulphinyl-C₁-C₂-alkyl, hetarylsulphonyl-C₁-C₂-alkyl, C₁-C₂-alkylthio-C₁-C₃-alkylcarbonyl, C₁-C₂-alkylsulphonyl-C₁-C₃-alkylcarbonyl, C₁-C₂-alkylthio-C₁-C₃-alkylamino-carbonyl, C₁-C₂-alkylsulphinyl-C₁-C₃-alkylaminocarbonyl, C₁-C₂-alkylsulphonyl-C₁-C₃-alkylaminocarbonyl, C₁-C₂-alkoxy-C₁-C₂-alkylcarbonyl, C₃-C₅-halocycloalkylcarbonyl-C₁-C₂-alkyl, C₁-C₃-alkoxycarbonyl-C₁-C₄-alkyl, C₁-C₂-alkoxycarbonyloxy-C₁-C₃-alkyl, C₁-C₃-alkylaminocarbonyl-C₁-C₂-alkyl, hydroxycarbonyl-C₁-C₂-alkyl, C₁-C₂-alkoxycarbonyl-C₁-C₄-alkylaminocarbonyl-C₁-C₂-alkyl, C₁-C₃-alkoxycarbonyl-di-(C₁-C₂)alkylaminocarbonyl-C₁-C₂-alkyl, hetaryl-C₂-C₃-haloalkenyl and aryl-C₁-C₂-alkylcarbonyl-C₁-C₂-alkyl,

where the aryl, hetaryl, aryl-C₁-C₂-alkyl, hetaryl-C₁-C₂-alkyl, hetaryl-C₁-C₃-haloalkyl, aryl-C₁-C₄-alkylaminocarbonyl, hetaryl-C₁-C₄-alkylaminocarbonyl, aryloxy-C₁-C₃-alkylaminocarbonyl, arylsulphonyl-(C₁-C₂-alkylamino)carbonyl, hetarylsulphonyl, aryloxycarbonyl, arylcarbonyl, hetarylcarbonyl, hetarylthio-C₁-C₂-alkyl, hetarylsulphinyl-C₁-C₂-alkyl, hetarylsulphonyl-C₁-C₂-alkyl, hetaryl-C₂-C₃-haloalkenyl and aryl-C₁-C₂-alkylcarbonyl-C₁-C₂-alkyl substituents are optionally mono- or polysubstituted, identically or differently, by halogen, cyano, C₁-C₈-alkyl, C₁-C₂-haloalkyl, di(C₁-C₂)alkylaminocarbonyl, C₁-C₃-alkoxycarbonyl, phenyl, C₁-C₂-alkoxy, C₁-C₂-haloalkoxy, C₁-C₃-haloalkylthio or COOH,

and where the heterocyclyl-C₁-C₂-alkyl, heterocyclyl-C₁-C₃-alkylaminocarbonyl, heterocyclylsulphonyl and heterocyclylcarbonyl substituents are optionally mono- or polysubstituted, identically or differently, by C₁-C₃-alkyl, C₃-C₅-cycloalkyl or C₁-C₂-alkoxy.

In the preferred definitions (range of preference (1)), unless stated otherwise:

halogen is selected from the group of fluorine, chlorine, bromine and iodine, preferably in turn from the group of fluorine, chlorine, bromine and iodine,

aryl (including as part of a larger unit, for example arylalkyl) is selected from the group of phenyl, naphthyl, anthryl, phenanthrenyl, and is preferably in turn phenyl,

hetaryl (synonymous with heteroaryl, including as part of a larger unit, for example hetarylalkyl) is selected from the group of furyl, thienyl, pyrrolyl, pyrazolyl, imidazolyl, triazolyl, oxazolyl, isoxazolyl, thiazolyl, isothiazolyl, oxadiazolyl, thiadiazolyl, tetrazolyl, pyridyl, pyrimidinyl, pyridazinyl, pyrazinyl, triazinyl, benzofuryl, benzoisofuryl, benzothienyl, benzoisothienyl, indolyl, isoindolyl, indazolyl, benzothiazolyl, benzoisothiazolyl, benzoxazolyl, benzisoxazolyl, benzimidazolyl, benzoxadiazolyl, quinolinyl, isoquinolinyl, cinnolinyl, phthalazinyl, quinazolinyl, quinoxalinyl, naphthyridinyl, benzotriazinyl, purinyl, pteridinyl, indolizinyl, imidazopyridinyl and benzothiadiazole.

Heterocyclyl is a saturated or unsaturated, non-heteroaromatic radical containing at least one 3- to 7-membered heterocyclic ring (=carbocyclic ring in which at least one carbon atom has been replaced by a heteroatom, preferably by a heteroatom from the group of N, O, S); this may be unsubstituted or substituted, in which case the bonding site is localized on a ring atom. Heterocyclyl is preferably selected from the group of aziridinyl, azirenyl, oxiranyl, oxirenyl, thiiranyl, thiirenyl, azetidinyl, dihydroazetyl, oxetanyl, oxetyl, thietanyl, thietyl, dioxetanyl, dithietanyl, oxathietanyl, pyrrolidinyl, dihydropyrrolyl, piperidinyl, tetrahydropyridinyl, dihydropyridinyl, azepanyl, tetrahydrofuranyl, dihydrofuranyl, tetrahydropyranyl, dihydropyranyl, pyranyl, oxepanyl, tetrahydrothiophenyl, dihydrothiophenyl, tetrahydrothiopyranyl, dihydrothiopyranyl, thiepanyl, dioxolanyl, dioxanyl, dioxepanyl, dithiolanyl, dithianyl, oxathiolanyl, oxathianyl, pyronyl, triazolonyl, triazolidindionyl, tetrahydropyrimidinonyl, thiadiazolopyrimidinonyl and 2-(N-cyanoimino)thiazolidinyl. If the heterocycle is a partly or fully saturated nitrogen-containing heterocycle, this may be joined to the rest of the molecule either via carbon or via the nitrogen. If sulphur occurs in rings in the above definitions, the sulphur may optionally also be in the form of SO or SO₂. Optionally, the heterocycle may be mono- or polysubstituted by oxygen or sulphur, which results in the formation of C═O or C═S groups.

In a preferred embodiment, these definitions also apply to the range of preference (5).

In the particularly preferred definitions (range of preference (2)), unless stated otherwise:

halogen is selected from the group of fluorine, chlorine, bromine and iodine,

aryl (including as part of a larger unit, for example arylalkyl) is selected from the group of phenyl, naphthyl, anthryl, phenanthrenyl, and is preferably in turn phenyl,

hetaryl (synonymous with heteroaryl, including as part of a larger unit, for example hetarylalkyl) is selected from the group of furyl, thienyl, pyrrolyl, pyrazolyl, imidazolyl, triazolyl, oxazolyl, isoxazolyl, thiazolyl, isothiazolyl, oxadiazolyl, thiadiazolyl, tetrazolyl, pyridyl, pyrimidinyl, pyridazinyl, pyrazinyl, imidazopyridinyl and benzothiadiazole.

Heterocyclyl is selected from the group of aziridinyl, oxiranyl, thiiranyl, azetidinyl, oxetanyl, thietanyl, pyrrolidinyl, dihydropyrrolyl, piperidinyl, tetrahydropyridinyl, dihydropyridinyl, azepanyl, tetrahydrofuranyl, dihydrofuranyl, tetrahydropyranyl, dihydropyranyl, pyranyl, oxepanyl, tetrahydrothiophenyl, dihydrothiophenyl, tetrahydrothiopyranyl, dihydrothiopyranyl, thiepanyl, dioxolanyl, dioxanyl, dioxepanyl, dithiolanyl, dithianyl, oxathiolanyl, oxathianyl, pyronyl, triazolonyl, triazolidindionyl, tetrahydropyrimidinonyl, thiadiazolopyrimidinonyl and 2-(N-cyanoimino)thiazolidinyl.

In a preferred embodiment, these definitions also apply to the range of preference (5).

In the very particularly preferred definitions (range of preference (3)), unless stated otherwise:

halogen is selected from the group of fluorine, chlorine, bromine and iodine,

aryl (including as part of a larger unit, for example arylalkyl) is selected from the group of phenyl, naphthyl, and is preferably in turn phenyl,

hetaryl (synonymous with heteroaryl, including as part of a larger unit, for example hetarylalkyl) is selected from the group of pyrazolyl, imidazolyl, triazolyl, thiazolyl, isothiazolyl, thiadiazolyl, tetrazolyl, pyridyl, pyrimidinyl, pyridazinyl, pyrazinyl, imidazopyridinyl and benzothiadiazole.

Heterocyclyl is selected from the group of aziridinyl, oxiranyl, thiiranyl, azetidinyl, oxetanyl, thietanyl, pyrrolidinyl, piperidinyl, tetrahydrofuranyl, tetrahydropyranyl, tetrahydrothiophenyl, tetrahydrothiopyranyl, dioxolanyl, dioxanyl, dithiolanyl, dithianyl, oxathiolanyl, oxathianyl, pyronyl, triazolonyl, triazolidindionyl, tetrahydropyrimidinonyl, thiadiazolopyrimidinonyl and 2-(N-cyanoimino)thiazolidinyl.

In a preferred embodiment, these definitions also apply to the range of preference (5).

In the explicitly very particularly preferred definitions (range of preference (4)), unless stated otherwise:

halogen is selected from the group of fluorine, chlorine and bromine,

aryl (including as part of a larger unit, for example arylalkyl) is phenyl,

hetaryl (synonymous with heteroaryl, including as part of a larger unit, for example hetarylalkyl) is selected from the group of pyrazolyl, imidazolyl, triazolyl, thiazolyl, isothiazolyl, thiadiazolyl, pyridyl, pyrimidinyl, pyridazinyl, pyrazinyl, imidazopyridinyl and benzothiadiazole.

Heterocyclyl is selected from the group of tetrahydrofuranyl, tetrahydropyranyl, tetrahydrothiophenyl, tetrahydrothiopyranyl, dioxolanyl, dioxanyl, dithiolanyl, dithianyl, pyronyl, triazolonyl, triazolidindionyl, tetrahydropyrimidinonyl, thiadiazolopyrimidinonyl and 2-(N-cyanoimino)thiazolidinyl.

In a preferred embodiment, these definitions also apply to the range of preference (5).

Halogen-substituted radicals, for example haloalkyl, are mono- or polyhalogenated, up to the maximum number of possible substituents. In the case of polyhalogenation, the halogen atoms may be the same or different. Halogen here is fluorine, chlorine, bromine and iodine.

Saturated or unsaturated hydrocarbyl radicals, for example alkyl or alkenyl, may each be straight-chain or branched as far as possible, including in combination with heteroatoms, as, for example, in alkoxy.

Optionally substituted radicals may be mono- or polysubstituted, where the substituents in the case of polysubstitution may be the same or different.

The radical definitions or elucidations given above in general terms or within areas of preference apply to the end products and correspondingly to the starting materials and intermediates. These radical definitions can be combined with one another as desired, i.e. including combinations between the respective preferred ranges.

Preference is given in accordance with the invention to using compounds of the formula (I) in which a combination of the definitions listed above as preferred is present (area of preference (1)).

Particular preference is given in accordance with the invention to using compounds of the formula (I) in which a combination of the definitions listed above as particularly preferred is present (area of preference (2)).

Very particular preference is given in accordance with the invention to using compounds of the formula (I) in which a combination of the definitions listed above as very particularly preferred is present (area of preference (3)).

Explicitly very particular preference is given in accordance with the invention to using compounds of the formula (I) in which a combination of the definitions listed above as explicitly very particularly preferred is present (area of preference (4)).

The invention also relates to novel compounds of the formula (I)

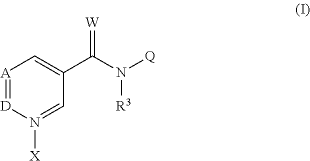

in which

A is N or CR¹,

D is N or CR², where A and D are not both N,

R¹ is a radical from the group of hydrogen, halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl,

R² is a radical from the group of hydrogen, halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl,

X is a free electron pair or oxygen,

W is oxygen, sulphur or NR⁴,

R³ is a radical from the group of hydrogen, cyano, hydroxyl, C₁-C₆-alkyl, C₃-C₆-cycloalkyl, C₁-C₆-haloalkyl, C₃-C₆-halocycloalkyl, C₂-C₆-alkenyl, C₂-C₆-haloalkenyl, C₃-C₆-alkynyl, C₃-C₆-haloalkynyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, C₁-C₆-alkylcarbonyl, C₂-C₆-alkenylcarbonyl, C₃-C₆-alkynylcarbonyl, C₁-C₆-haloalkylcarbonyl, C₃-C₆-cycloalkylcarbonyl, C₃-C₆-cycloalkyl-C₁-C₆-alkylcarbonyl, C₂-C₆-haloalkenylcarbonyl, C₃-C₆-halocycloalkylcarbonyl, C₁-C₆-alkoxy-C₁-C₆-alkylcarbonyl, C₁-C₆-alkoxycarbonyl, C₁-C₆-haloalkoxycarbonyl, C₁-C₆-alkylsulphonyl, C₁-C₆-haloalkylsulphonyl, C₁-C₆-alkylaminocarbonyl, di(C₁-C₆)alkylaminocarbonyl, C₁-C₆-alkylcarbonyloxy, C₁-C₆-alkoxycarbonyloxy, aryl, hetaryl, arylcarbonyl, hetarylcarbonyl, aryl-C₁-C₆-alkyl, hetaryl-C₁-C₆-alkyl, aryl-C₁-C₆-alkylcarbonyl, hetaryl-C₁-C₆-alkylcarbonyl, where the aryl, hetaryl, aryl-C₁-C₆-alkyl, hetaryl-C₁-C₆-alkyl, arylcarbonyl, hetarylcarbonyl, aryl-C₁-C₆-alkylcarbonyl, hetaryl-C₁-C₆-alkylcarbonyl substituents are optionally mono- or polysubstituted, identically or differently, by halogen, cyano, nitro, hydroxyl, C₁-C₆-alkyl, C₂-C₆-alkenyl, C₃-C₆-alkynyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl,

R⁴ is a radical from the group of hydrogen, cyano, nitro, hydroxyl, C₁-C₆-alkyl, C₃-C₆-cycloalkyl, C₁-C₆-haloalkyl, C₃-C₆-halocycloalkyl, C₂-C₆-alkenyl, C₂-C₆-haloalkenyl, C₃-C₆-alkynyl, C₃-C₆-haloalkynyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, C₁-C₆-alkylcarbonyl, C₁-C₆-haloalkylcarbonyl, C₁-C₆-alkoxycarbonyl, C₁-C₆-alkylcarbonyloxy, C₁-C₆-alkylsulphonyl, C₁-C₆-haloalkylsulphonyl, aryl, hetaryl, aryl-C₁-C₆-alkyl, hetaryl-C₁-C₆-alkyl, arylcarbonyl, hetarylcarbonyl, where the aryl, hetaryl, aryl-C₁-C₆-alkyl, hetaryl-C₁-C₆-alkyl, arylcarbonyl, hetarylcarbonyl substituents are optionally mono- or polysubstituted, identically or differently, by halogen, cyano, nitro, hydroxyl, C₁-C₆-alkyl, C₂-C₆-alkenyl, C₃-C₆-alkynyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl,

Q is one of the Q-1 to Q-4 radicals:

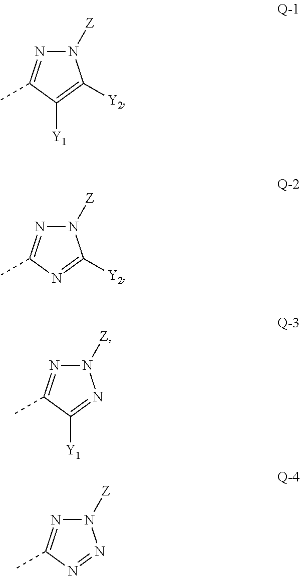

in which the dotted line is the bond to the nitrogen in the NR³ group,

Y₁ is a radical from the group of hydrogen, halogen, cyano, nitro, C₁-C₆-alkylamino, di(C₁-C₆)alkylamino, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di(C₁-C₆)alkylaminocarbonyl,

Y₂ is a radical from the group of hydrogen, halogen, cyano, nitro, C₁-C₆-alkylamino, di(C₁-C₆)alkylamino, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, SH, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di(C₁-C₆)alkylaminocarbonyl,

Z is a radical from the group of hydrogen, cyano, nitro, L¹(=G)C*, L¹O(=G)C*, L³L⁴N(=G)C*, L⁵(=E)CL²N(=G)C*, L⁵S(═O)ₙL²N(=G)C*, L⁵O(=E)CL²N(=G)C*, L¹L⁵N(=E)CL²N(=G)C*, L¹(═O)ₙS*, L³L⁴N(═O)ₙS*, heterocyclyl, aryl, hetaryl, optionally singly or multiply, identically or differently M²-substituted C₁-C₈-alkyl, C₃-C₈-cycloalkyl, C₂-C₈-alkenyl, C₅-C₈-cycloalkenyl, C₃-C₈-alkynyl, optionally singly or multiply, identically or differently M⁶-substituted (in the —C₁-C₈-alkyl group) L⁵C(=E)L²N(=G)C—C₁-C₈-alkyl*, L⁵S(═O)ₙL²N(=G)C—C₁-C₈-alkyl*, L⁵OC(=E)L²N(=G)C—C₁-C₈-alkyl*, L¹L⁵NC(=E)L²N(=G)C—C₁-C₈-alkyl*, L¹(═O)ₙS—C₁-C₈-alkyl*, L³O(═O)ₙS—C₁-C₈-alkyl*, L³L⁴N(═O)ₙS—C₁-C₈-alkyl*,

with the proviso that, when Q is Q-4, Z is not simultaneously hydrogen or unsubstituted C₁-C₈-alkyl, and with the proviso that Z is not hydrogen when W is NOH, and with the proviso that, when Q is Q-2, Z is not simultaneously hydrogen,

M² is halogen, cyano, nitro, NL⁹L¹⁰, OL⁹, SL⁹, L¹(=G)C*, L³O(=G)C*, L³L⁴N(=G)C*, heterocyclyl, aryl, hetaryl, optionally singly or multiply, identically or differently, M³-substituted C₁-C₆-alkyl, C₃-C₆-cycloalkyl, C₂-C₆-alkenyl, C₅-C₆-cycloalkenyl, C₃-C₆-alkynyl, C₁-C₆-alkoxy,

M³ is halogen, cyano, nitro, heterocyclyl, aryl, hetaryl, NL⁹L¹⁰, OL⁹, SL⁹, C₁-C₆-alkyl, C₁-C₆-haloalkyl, C₃-C₆-cycloalkyl, C₃-C₆-halocycloalkyl, C₂-C₆-alkenyl, C₃-C₆-alkynyl, C₁-C₆-alkoxy, C₁-C₆-haloalkoxy, L′(=G)C*, L³O(=G)C*, L³L⁴N(=G)C*,

G and E are each independently oxygen or sulphur,

n is 1 or 2,

L¹ and L⁵ are each independently heterocyclyl, aryl, hetaryl, optionally singly or multiply, identically or differently, M⁴-substituted C₁-C₆-alkyl, C₃-C₆-cycloalkyl, C₃-C₆-cycloalkyl-C₁-C₆-alkyl, C₂-C₆-alkenyl, C₃-C₆-alkynyl,

M⁴ is halogen, cyano, nitro, heterocyclyl, aryl, hetaryl, C₁-C₆-haloalkyl, C₁-C₆-alkoxy, C₁-C₆-haloalkoxy, NL⁹L¹⁰, OL⁹, SL⁹,

L² is hydrogen, aryl, hetaryl, optionally singly or multiply, identically or differently, M⁵-substituted C₁-C₆-alkyl, C₃-C₆-cycloalkyl, C₃-C₆-cycloalkyl-C₁-C₆-alkyl, C₂-C₆-alkenyl, C₃-C₆-alkynyl,

L³ and L⁴ are each independently hydrogen, heterocyclyl, aryl, hetaryl, optionally singly or multiply, identically or differently, M⁵-substituted C₁-C₆-alkyl, C₃-C₆-cycloalkyl, C₃-C₆-cycloalkyl-C₁-C₆-alkyl, C₂-C₆-alkenyl, C₃-C₆-alkynyl,

M⁵ is halogen, cyano, nitro, heterocyclyl, aryl, hetaryl, C₁-C₆-haloalkyl, C₁-C₆-alkoxy, C₁-C₆-haloalkoxy, C₁-C₆-alkoxycarbonyl, NL⁹L¹⁰, OL⁹, SL⁹,

M⁶ is halogen, cyano, nitro, NL⁹L¹⁰, OL⁹, SL⁹, L¹(=G)C*, L³O(=G)C*, L³L⁴N(=G)C*, heterocyclyl, aryl, hetaryl, C₁-C₆-alkyl, C₁-C₆-haloalkyl, C₃-C₆-cycloalkyl, C₃-C₆-halocycloalkyl, C₃-C₆-cycloalkyl-C₁-C₆-alkyl, C₃-C₆-halocycloalkyl-C₁-C₆-alkyl, C₂-C₆-alkenyl, C₅-C₆-cycloalkenyl, C₃-C₆-alkynyl, C₁-C₆-alkoxy-C₁-C₆-alkyl, C₁-C₆-haloalkoxy-C₁-C₆-alkyl,

L⁹ and L¹⁰ are each independently hydrogen, C₁-C₆-alkyl, C₁-C₆-haloalkyl, C₃-C₆-cycloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₂-C₆-alkenyl, C₂-C₆-haloalkenyl, C₃-C₆-alkynyl, C₁-C₆-alkoxy-C₁-C₆-alkyl, C₁-C₆-haloalkoxy-C₁-C₆-alkyl, C₁-C₆-alkylcarbonyl, C₂-C₆-alkenylcarbonyl, C₃-C₆-alkynylcarbonyl, C₁-C₆-haloalkylcarbonyl, C₃-C₆-cycloalkylcarbonyl, C₃-C₆-cycloalkyl-C₁-C₆-alkylcarbonyl, C₂-C₆-haloalkenylcarbonyl, C₃-C₆-halocycloalkylcarbonyl, C₁-C₆-alkoxycarbonyl, C₁-C₆-alkoxy-C₁-C₆-alkylcarbonyl, C₁-C₆-haloalkoxycarbonyl, C₃-C₆-cycloalkoxycarbonyl, aminocarbonyl, C₁-C₆-alkylaminocarbonyl, di(C₁-C₆)alkylaminocarbonyl, C₃-C₆-cycloalkylaminocarbonyl, C₃-C₆-cycloalkyl-C₁-C₆-alkylaminocarbonyl, aryl, hetaryl, heterocyclyl, aryl-C₁-C₆-alkyl, hetaryl-C₁-C₆-alkyl,

in which, unless stated otherwise, the heterocyclyl, aryl and hetaryl radicals (including as part of a larger unit such as aryloxy, hetarylalkyl etc.) are optionally mono- or polysubstituted, identically or differently, by halogen, cyano, nitro, SH, hydroxyl, C₁-C₆-alkyl, C₂-C₆-alkenyl, C₃-C₆-alkynyl, C₃-C₆-cycloalkyl, C₁-C₆-haloalkyl, cyano-C₁-C₆-alkyl, C₂-C₆-haloalkenyl, C₃-C₆-haloalkynyl, C₁-C₆-alkoxy, C₂-C₆-alkenyloxy, C₃-C₆-alkynyloxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₃-C₆-cycloalkyl-C₁-C₆-alkyl, C₃-C₆-halocycloalkyl-C₁-C₆-alkyl, amino, C₁-C₆-alkylamino, di(C₁-C₆)alkylamino, C₃-C₆-cycloalkylamino, C₃-C₆-cycloalkyl-C₁-C₆-alkylamino, C₁-C₆-alkylcarbonylamino, C₁-C₆-haloalkylcarbonylamino, C₁-C₆-cycloalkylcarbonylamino, CO₂H, SO₃H, CONH₂, SO₂NH₂, C₁-C₆-alkylcarbonyl, C₂-C₆-alkenylcarbonyl, C₃-C₆-alkynylcarbonyl, C₁-C₆-haloalkylcarbonyl, C₃-C₆-cycloalkylcarbonyl, C₃-C₆-cycloalkyl-C₁-C₆-alkylcarbonyl, C₂-C₆-haloalkenylcarbonyl, C₃-C₆-halocycloalkylcarbonyl, C₁-C₆-alkoxycarbonyl, C₁-C₆-alkoxy-C₁-C₆-alkylcarbonyl, C₁-C₆-haloalkoxycarbonyl, C₃-C₆-cycloalkoxycarbonyl, C₁-C₆-alkylthio, C₁-C₆-haloalkylthio, C₁-C₆-alkylsulphinyl, C₁-C₆-haloalkylsulphinyl, C₁-C₆-alkylsulphonyl, C₁-C₆-haloalkylsulphonyl, C₁-C₆-alkylaminosulphonyl, C₁-C₆-haloalkylaminosulphonyl, di(C₁-C₆)alkylaminosulphonyl, C₁-C₆-alkylaminocarbonyl, di(C₁-C₆)alkylaminocarbonyl, C₁-C₆-alkylcarbonyloxy, C₁-C₆-haloalkylcarbonyloxy, C₁-C₆-alkoxycarbonyloxy, C₁-C₆-haloalkoxycarbonyloxy, aryl, hetaryl, aryloxy, hetaryloxy,

with the proviso that, when Y₂ is methyl and Z is phenyl-C₁-C₆-alkyl, the phenyl ring in the ortho position is not substituted by alkoxy or by arylalkoxy, with the proviso that Z is not 3,5-dimethyl-1,2-oxazol-4-ylmethyl when A and D are each CH and, at the same time, X is a free electron pair, W is oxygen, Q is Q-1 and R³, Y₁ and Y₂ are each hydrogen, and with the proviso that compounds of the formulae (G1), (G2) and (G4) are excluded.

Preferred substituents or ranges for the radicals shown in the novel compounds of the formula (I) are elucidated below (area of preference (6)).

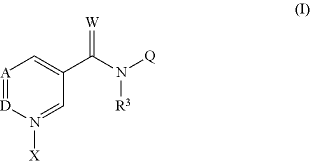

A is Nor CR¹.

D is N or CR², where A and D are not both N.

R¹ is a radical from the group of hydrogen, halogen, cyano, nitro, C₁-C₃-alkyl, C₁-C₃-haloalkyl, C₁-C₃-alkoxy, C₁-C₃-haloalkoxy.

R² is a radical from the group of hydrogen, halogen, cyano, nitro, C₁-C₃-alkyl, C₁-C₃-haloalkyl, C₁-C₃-alkoxy, C₁-C₃-haloalkoxy.

X is a free electron pair or oxygen.

W is oxygen, sulphur or NR⁴.

R³ is a radical from the group of hydrogen, cyano, C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, C₁-C₆-alkylcarbonyl, C₁-C₆-haloalkylcarbonyl, C₃-C₆-cycloalkylcarbonyl, C₃-C₆-cycloalkyl-C₁-C₂-alkylcarbonyl, C₃-C₆-halocycloalkylcarbonyl, C₁-C₆-alkoxy-C₁-C₆-alkylcarbonyl, C₁-C₆-alkoxycarbonyl, C₁-C₆-haloalkoxycarbonyl, C₁-C₃-alkylsulphonyl, C₁-C₃-haloalkylsulphonyl, C₁-C₃-alkylaminocarbonyl, di(C₁-C₃)alkylaminocarbonyl, aryl, hetaryl, arylcarbonyl, hetarylcarbonyl, aryl-C₁-C₆-alkyl, hetaryl-C₁-C₆-alkyl, aryl-C₁-C₆-alkylcarbonyl, hetaryl-C₁-C₆-alkylcarbonyl, where the aryl, hetaryl, aryl-C₁-C₆-alkyl, hetaryl-C₁-C₆-alkyl, arylcarbonyl, hetarylcarbonyl, aryl-C₁-C₆-alkylcarbonyl, hetaryl-C₁-C₆-alkylcarbonyl substituents are optionally singly or multiply substituted, identically or differently, by halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl.

R⁴ is a radical from the group of hydrogen, cyano, nitro, C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, C₁-C₆-alkylcarbonyl, C₁-C₆-haloalkylcarbonyl, C₁-C₆-alkoxycarbonyl, C₁-C₆-alkylcarbonyloxy, C₁-C₃-alkylsulphonyl, C₁-C₃-haloalkylsulphonyl, aryl, hetaryl, aryl-C₁-C₆-alkyl, hetaryl-C₁-C₆-alkyl, arylcarbonyl, hetarylcarbonyl, where the aryl, hetaryl, aryl-C₁-C₆-alkyl, hetaryl-C₁-C₆-alkyl, arylcarbonyl, hetarylcarbonyl substituents are optionally singly or multiply substituted, identically or differently, by halogen, cyano, nitro, C₁-C₄-alkyl, C₁-C₄-haloalkyl, C₁-C₃-alkoxy, C₁-C₃-haloalkoxy, C₁-C₆-alkoxy-C₁-C₂-alkyl.

Q is one of the Q-1 to Q-4 radicals:

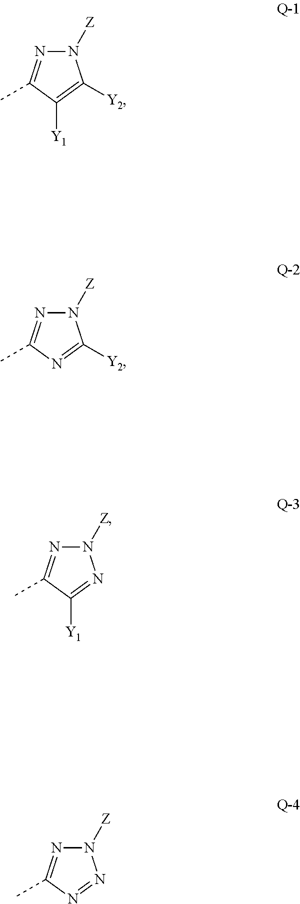

in which the dotted line is the bond to the nitrogen in the NR³ group.

Y₁ is a radical from the group of hydrogen, halogen, cyano, C₁-C₃-alkylamino, di(C₁-C₃)alkylamino, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₂-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl-C₁-C₆-alkylthio, C₁-C₆-alkylcarbonyl, C₃-C₆-cycloalkylcarbonyl, C₂-C₄-alkenylcarbonyl, C₁-C₆-haloalkylcarbonyl, C₁-C₃-alkoxycarbonyl, C₁-C₆-alkylsulphonyl, C₁-C₃-haloalkylsulphonyl, C₁-C₃-alkylaminocarbonyl, di(C₁-C₃)alkylaminocarbonyl.

Y₂ is a radical from the group of hydrogen, halogen, cyano, C₁-C₃-alkylamino, di(C₁-C₃)alkylamino, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₂-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₂-alkyl, SH, C₁-C₆-alkylthio, C₁-C₆-alkylcarbonyl, C₃-C₆-cycloalkylcarbonyl, C₂-C₄-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₃-alkylaminocarbonyl, di(C₁-C₃)alkylaminocarbonyl.

Z is a radical from the group of hydrogen, cyano, L¹(=G)C*, L¹O(=G)C*, L³L⁴N(=G)C*, L⁵(=E)CL²N(=G)C*, L⁵(═O)ₙSL²N(=G)C*, L⁵O(=E)CL²N(=G)C*, L¹L⁵N(=E)CL²N(=G)C*, L¹(═O)ₙS*, L³L⁴N(═O)ₙS*, heterocyclyl, aryl, hetaryl, optionally singly or multiply, identically or differently M²-substituted C₁-C₆-alkyl, C₃-C₆-cycloalkyl, C₂-C₆-alkenyl, C₃-C₆-alkynyl, optionally singly or multiply, identically or differently M⁶-substituted (in the —C₁-C₆-alkyl group) L⁵C(=E)L²N(=G)C—C₁-C₆-alkyl*, L⁵(═O)ₙSL²N(=G)C—C₁-C₆-alkyl*, L⁵OC(=E)L²N(=G)C—C₁-C₆-alkyl*, L¹L⁵NC(=E)L²N(=G)C—C₁-C₆-alkyl*, L¹(═O)ₙS—C₁-C₆-alkyl*, L³O(═O)ₙS—C₁-C₆-alkyl*, L³L⁴N(═O)ₙS—C₁-C₆-alkyl*,

with the proviso that, when Q is Q-4, Z is not simultaneously hydrogen or unsubstituted C₁-C₈-alkyl, and with the proviso that, when Q is Q-2, Z is not simultaneously hydrogen.

M² is halogen, cyano, nitro, NL⁹L¹⁰, OL⁹, SL⁹, L¹(=G)C*, L³O(=G)C*, L³L⁴N(=G)C*, heterocyclyl, aryl, hetaryl, optionally singly or multiply, identically or differently, M³-substituted C₁-C₆-alkyl, C₃-C₆-cycloalkyl, C₂-C₆-alkenyl, C₃-C₆-alkynyl, C₁-C₆-alkoxy.

M³ is halogen, cyano, nitro, heterocyclyl, aryl, hetaryl, NL⁹L¹⁰, OL⁹, SL⁹, C₁-C₆-alkyl, C₁-C₆-haloalkyl, C₃-C₆-cycloalkyl, C₃-C₆-halocycloalkyl, C₂-C₆-alkenyl, C₃-C₆-alkynyl, C₁-C₆-alkoxy, C₁-C₆-haloalkoxy, L¹(=G)C*, L³O(=G)C*, L³L⁴N(=G)C*.

G and E are each independently oxygen or sulphur.

n is 1 or 2.

L¹ and L⁵ are each independently heterocyclyl, aryl, hetaryl, optionally singly or multiply, identically or differently, M⁴-substituted C₁-C₆-alkyl, C₃-C₆-cycloalkyl, C₃-C₆-cycloalkyl-C₁-C₆-alkyl, C₂-C₆-alkenyl, C₃-C₆-alkynyl.

M⁴ is halogen, cyano, nitro, heterocyclyl, aryl, hetaryl, C₁-C₆-haloalkyl, C₁-C₆-alkoxy, C₁-C₆-haloalkoxy, NL⁹L¹⁰, OL⁹, SL⁹.

L² is hydrogen, aryl, hetaryl, optionally singly or multiply, identically or differently, M⁵-substituted C₁-C₆-alkyl, C₃-C₆-cycloalkyl, C₃-C₆-cycloalkyl-C₁-C₆-alkyl, C₂-C₆-alkenyl, C₃-C₆-alkynyl.

L³ and L⁴ are each independently hydrogen, heterocyclyl, aryl, hetaryl, optionally singly or multiply, identically or differently, M⁵-substituted C₁-C₆-alkyl, C₃-C₆-cycloalkyl, C₃-C₆-cycloalkyl-C₁-C₆-alkyl, C₂-C₆-alkenyl, C₃-C₆-alkynyl.

M⁵ is halogen, cyano, nitro, heterocyclyl, aryl, hetaryl, C₁-C₆-haloalkyl, C₁-C₆-alkoxy, C₁-C₆-haloalkoxy, C₁-C₆-alkoxycarbonyl, NL⁹L¹⁰, OL⁹, SL⁹.

M⁶ is halogen, cyano, nitro, NL⁹L¹⁰, OL⁹, SL⁹, L¹(=G)C*, L³O(=G)C*, L³L⁴N(=G)C*, heterocyclyl, aryl, hetaryl, C₁-C₆-alkyl, C₁-C₆-haloalkyl, C₃-C₆-cycloalkyl, C₃-C₆-halocycloalkyl, C₃-C₆-cycloalkyl-C₁-C₆-alkyl, C₃-C₆-halocycloalkyl-C₁-C₆-alkyl, C₂-C₆-alkenyl, C₃-C₆-alkynyl, C₁-C₆-alkoxy-C₁-C₃-alkyl, C₁-C₆-haloalkoxy-C₁-C₃-alkyl.

L⁹ and L¹⁰ are each independently hydrogen, C₁-C₆-alkyl, C₁-C₆-haloalkyl, C₃-C₆-cycloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₂-C₄-alkenyl, C₂-C₄-haloalkenyl, C₃-C₆-alkynyl, C₁-C₆-alkoxy-C₁-C₆-alkyl, C₁-C₆-haloalkoxy-C₁-C₆-alkyl, C₁-C₆-alkylcarbonyl, C₂-C₄-alkenylcarbonyl, C₃-C₆-alkynylcarbonyl, C₁-C₆-haloalkylcarbonyl, C₃-C₆-cycloalkylcarbonyl, C₃-C₆-cycloalkyl-C₁-C₆-alkylcarbonyl, C₂-C₄-haloalkenylcarbonyl, C₃-C₆-halocycloalkylcarbonyl, C₁-C₆-alkoxycarbonyl, C₁-C₆-alkoxy-C₁-C₆-alkylcarbonyl, C₁-C₆-haloalkoxycarbonyl, C₃-C₆-cycloalkoxycarbonyl, aminocarbonyl, C₁-C₆-alkylaminocarbonyl, di(C₁-C₆)alkylaminocarbonyl, C₃-C₆-cycloalkylaminocarbonyl, C₃-C₆-cycloalkyl-C₁-C₆-alkylaminocarbonyl, aryl, hetaryl, heterocyclyl, aryl-C₁-C₆-alkyl, hetaryl-C₁-C₆-alkyl.

In this context, when Y₂ is methyl and Z is phenyl-C₁-C₆-alkyl, the phenyl ring in the ortho position is not substituted by alkoxy or by arylalkoxy; in addition, Z is not 3,5-dimethyl-1,2-oxazol-4-ylmethyl when A and D are each CH and, at the same time, X is a free electron pair, W is oxygen, Q is Q-1 and R³, Y₁ and Y₂ are each hydrogen; and finally, compounds of the formulae (G1), (G2) and (G4) are excluded.

In the preferred substituents or ranges mentioned, unless stated otherwise, the heterocyclyl, aryl and hetaryl radicals (including as part of a larger unit such as aryloxy, hetarylalkyl etc.) are optionally mono- or polysubstituted, identically or differently, by halogen, cyano, nitro, SH, hydroxyl, C₁-C₆-alkyl, C₃-C₆-cycloalkyl, C₁-C₆-haloalkyl, cyano-C₁-C₆-alkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₃-C₆-cycloalkyl-C₁-C₆-alkyl, C₃-C₆-halocycloalkyl-C₁-C₆-alkyl, amino, C₁-C₆-alkylamino, di(C₁-C₆)alkylamino, C₃-C₆-cycloalkylamino, C₃-C₆-cycloalkyl-C₁-C₆-alkylamino, C₁-C₆-alkylcarbonylamino, C₁-C₆-haloalkylcarbonylamino, C₁-C₆-cycloalkylcarbonylamino, CO₂H, SO₃H, CONH₂, SO₂NH₂, C₁-C₆-alkylcarbonyl, C₂-C₆-alkenylcarbonyl, C₃-C₆-alkynylcarbonyl, C₁-C₆-haloalkylcarbonyl, C₃-C₆-cycloalkylcarbonyl, C₃-C₆-cycloalkyl-C₁-C₆-alkylcarbonyl, C₂-C₆-haloalkenylcarbonyl, C₃-C₆-halocycloalkylcarbonyl, C₁-C₆-alkoxycarbonyl, C₁-C₆-alkoxy-C₁-C₆-alkylcarbonyl, C₁-C₆-haloalkoxycarbonyl, C₃-C₆-cycloalkoxycarbonyl, C₁-C₆-alkylthio, C₁-C₆-haloalkylthio, C₁-C₆-alkylsulphinyl, C₁-C₆-haloalkylsulphinyl, C₁-C₆-alkylsulphonyl, C₁-C₆-haloalkylsulphonyl, C₁-C₆-alkylaminosulphonyl, C₁-C₆-haloalkylaminosulphonyl, di(C₁-C₆)alkylaminosulphonyl, C₁-C₆-alkylaminocarbonyl, di(C₁-C₆)alkylaminocarbonyl, C₁-C₆-alkylcarbonyloxy, C₁-C₆-haloalkylcarbonyloxy, C₁-C₆-alkoxycarbonyloxy, C₁-C₆-haloalkoxycarbonyloxy, aryl, hetaryl, aryloxy, hetaryloxy.

Particularly preferred substituents or ranges for the radicals shown in the novel compounds of the formula (I) are elucidated below (area of preference (7)).

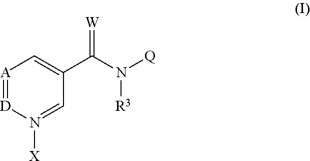

A is Nor CR¹.

D is N or CR², where A and D are not both N.

R¹ is a radical from the group of hydrogen, halogen, cyano, methyl, halomethyl, methoxy, halomethoxy.

R² is a radical from the group of hydrogen, halogen, cyano, methyl, halomethyl, methoxy, halomethoxy.

X is a free electron pair or oxygen.

W is oxygen, sulphur or NR⁴.

R³ is a radical from the group of hydrogen, C₁-C₆-alkyl, C₃-C₆-cycloalkyl, C₁-C₆-haloalkyl, C₃-C₆-halocycloalkyl, C₃-C₆-cycloalkyl-C₁-C₆-alkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkylcarbonyl, C₁-C₆-haloalkylcarbonyl, C₃-C₆-cycloalkylcarbonyl, C₁-C₆-alkoxy-C₁-C₆-alkylcarbonyl, C₁-C₆-alkoxycarbonyl, C₁-C₆-haloalkoxycarbonyl, aryl, hetaryl, arylcarbonyl, hetarylcarbonyl, where the aryl, hetaryl, arylcarbonyl and hetarylcarbonyl substituents are optionally singly or multiply substituted, identically or differently, by halogen, cyano, C₁-C₃-alkyl, C₁-C₃-haloalkyl, C₁-C₃-alkoxy, C₁-C₃-haloalkoxy, C₁-C₃-alkoxy-C₁-C₂-alkyl.

R⁴ is a radical from the group of hydrogen, cyano, nitro, C₁-C₃-alkyl, C₃-C₆-cycloalkyl, C₁-C₃-haloalkyl, C₃-C₆-halocycloalkyl, C₃-C₆-cycloalkyl-C₁-C₃-alkyl, C₃-C₆-halocycloalkyl-C₁-C₃-alkyl, cyano-C₁-C₃-alkyl, C₁-C₃-alkoxy, C₁-C₃-haloalkoxy, C₁-C₃-alkoxy-C₁-C₃-alkyl, C₁-C₃-haloalkoxy-C₁-C₃-alkyl, C₁-C₄-alkylcarbonyl, C₁-C₄-haloalkylcarbonyl, C₁-C₃-alkoxycarbonyl, C₁-C₃-alkylcarbonyloxy.

Q is one of the Q-1 to Q-3 radicals:

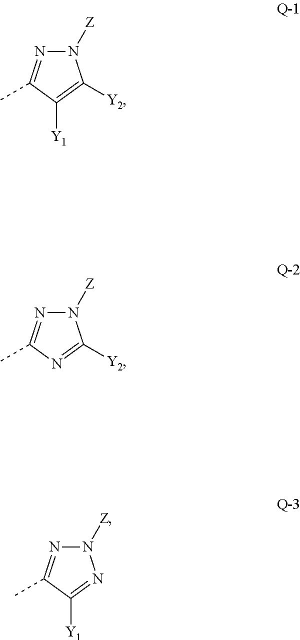

in which the dotted line is the bond to the nitrogen in the NR³ group.

Y₁ is a radical from the group of hydrogen, halogen, cyano, C₁-C₆-alkyl, C₃-C₆-cycloalkyl, C₁-C₆-haloalkyl, C₁-C₆-alkoxy, C₁-C₆-haloalkoxy, C₁-C₆-alkoxy-C₁-C₃-alkyl, C₁-C₆-haloalkoxy-C₁-C₃-alkyl, C₁-C₆-alkoxy-C₁-C₃-alkyloxy, cyano-C₁-C₃-alkyl, C₃-C₆-cycloalkyl-C₁-C₃-alkyl, C₁-C₃-alkylthio, C₁-C₄-alkylcarbonyl, C₁-C₄-haloalkylcarbonyl, C₁-C₃-alkylsulphonyl.

Y₂ is a radical from the group of hydrogen, halogen, cyano, C₁-C₆-alkyl, C₃-C₆-cycloalkyl, C₁-C₆-haloalkyl, C₁-C₆-alkoxy, C₁-C₆-haloalkoxy, C₁-C₆-alkoxy-C₁-C₄-alkyl, C₁-C₆-haloalkoxy-C₁-C₄-alkyl, C₁-C₆-alkoxy-C₁-C₄-alkyloxy, cyano-C₁-C₃-alkyl, C₃-C₆-cycloalkyl-C₁-C₂-alkyl, SH, C₁-C₃-alkylthio, C₁-C₄-alkylcarbonyl, C₁-C₄-haloalkylcarbonyl, C₁-C₄-alkoxycarbonyl, C₁-C₃-alkylsulphonyl, C₁-C₃-haloalkylsulphonyl.

Z is a radical from the group of hydrogen, L¹(=G)C*, L¹O(=G)C*, L³L⁴N(=G)C*, L⁵(=E)CL²N(=G)C*, L⁵(═O)ₙSL²N(=G)C*, L⁵O(=E)CL²N(=G)C*, L¹L⁵N(=E)CL²N(=G)C*, L¹(═O)ₙS*, L³L⁴N(═O)ₙS*, heterocyclyl, aryl, hetaryl, optionally singly or multiply, identically or differently M²-substituted C₁-C₆-alkyl, C₃-C₆-cycloalkyl, C₂-C₆-alkenyl, C₃-C₆-alkynyl, optionally singly or multiply, identically or differently M⁶-substituted (in the —C₁-C₄-alkyl group) L⁵C(=E)L²N(=G)C—C₁-C₄-alkyl*, L⁵(═O)ₙSL²N(=G)C—C₁-C₄-alkyl*, L⁵OC(=E)L²N(=G)C—C₁-C₄-alkyl*, L¹L⁵NC(=E)L²N(=G)C—C₁-C₄-alkyl*, L¹ (═O)ₙS—C₁-C₄-alkyl*, L³O(═O)ₙS—C₁-C₄-alkyl*, L³L⁴N(═O)ₙS—C₁-C₄-alkyl*,

with the proviso that, when Q is Q-2, Z is not simultaneously hydrogen.

M² is halogen, cyano, NL⁹L¹⁰, OL⁹, SL⁹, L¹(=G)C*, L³O(=G)C*, L³L⁴N(=G)C*, heterocyclyl, aryl, hetaryl, C₁-C₄-alkyl, C₁-C₄-haloalkyl, C₁-C₄-cyanoalkyl, C₃-C₆-cycloalkyl, C₃-C₆-halocycloalkyl.

G and E are each independently oxygen or sulphur.

n is 1 or 2.

L¹ and L⁵ are each independently heterocyclyl, aryl, hetaryl, optionally singly or multiply, identically or differently, M⁴-substituted C₁-C₆-alkyl, aryl-C₁-C₄-alkyl, hetaryl-C₁-C₄-alkyl, C₃-C₆-cycloalkyl, C₃-C₆-cycloalkyl-C₁-C₄-alkyl, C₂-C₆-alkenyl, C₃-C₆-alkynyl.

M⁴ is halogen, cyano, C₁-C₃-haloalkyl, C₁-C₃-alkoxy, C₁-C₃-haloalkoxy.

L² is hydrogen, aryl, hetaryl, optionally singly or multiply, identically or differently, M⁵-substituted C₁-C₆-alkyl, aryl-C₁-C₄-alkyl, hetaryl-C₁-C₄-alkyl, C₃-C₆-cycloalkyl, C₃-C₆-cycloalkyl-C₁-C₄-alkyl.

L³ and L⁴ are each independently hydrogen, heterocyclyl, aryl, hetaryl, optionally singly or multiply, identically or differently, M⁵-substituted C₁-C₆-alkyl, aryl-C₁-C₄-alkyl, hetaryl-C₁-C₄-alkyl, C₃-C₆-cycloalkyl, C₃-C₆-cycloalkyl-C₁-C₄-alkyl.

M⁵ is halogen, cyano, C₁-C₆-haloalkyl, C₁-C₆-alkoxy, C₁-C₆-haloalkoxy, C₁-C₄-alkoxycarbonyl.

M⁶ is halogen, cyano, NL⁹L¹⁰, OL⁹, aryl, hetaryl, C₁-C₃-haloalkyl, C₃-C₄-cycloalkyl, C₃-C₄-halocycloalkyl, C₃-C₄-cycloalkyl-C₁-C₃-alkyl, C₃-C₄-halocycloalkyl-C₁-C₃-alkyl.

L⁹ and L¹⁰ are each independently hydrogen, C₁-C₆-alkyl, C₁-C₆-haloalkyl, C₃-C₆-cycloalkyl, C₃-C₆-halocycloalkyl, C₃-C₆-cycloalkyl-C₁-C₄-alkyl, C₃-C₆-halocycloalkyl-C₁-C₄-alkyl, cyano-C₁-C₆-alkyl, C₁-C₆-alkoxy-C₁-C₄-alkyl, C₁-C₆-haloalkoxy-C₁-C₄-alkyl, C₁-C₆-alkylcarbonyl, C₁-C₆-haloalkylcarbonyl, C₃-C₆-cycloalkylcarbonyl, C₃-C₆-cycloalkyl-C₁-C₄-alkylcarbonyl, C₃-C₆-halocycloalkylcarbonyl, C₁-C₆-alkoxycarbonyl, C₁-C₆-alkoxy-C₁-C₄-alkylcarbonyl, C₁-C₆-haloalkoxycarbonyl, C₃-C₆-cycloalkoxycarbonyl, C₁-C₆-alkylaminocarbonyl, di(C₁-C₆)alkylaminocarbonyl, C₃-C₆-cycloalkylaminocarbonyl, C₃-C₆-cycloalkyl-C₁-C₆-alkylaminocarbonyl, aryl, hetaryl, heterocyclyl, aryl-C₁-C₆-alkyl, hetaryl-C₁-C₆-alkyl.

In this context, when Y₂ is methyl and Z is phenyl-C₁-C₆-alkyl, the phenyl ring in the ortho position is not substituted by alkoxy or by arylalkoxy; in addition, Z is not 3,5-dimethyl-1,2-oxazol-4-ylmethyl when A and D are each CH and, at the same time, X is a free electron pair, W is oxygen, Q is Q-1 and R³, Y₁ and Y₂ are each hydrogen; and finally, compounds of the formulae (G1) and (G2) are excluded.

In the particularly preferred substituents or ranges mentioned, unless stated otherwise, the heterocyclyl, aryl and hetaryl radicals (including as part of a larger unit such as aryloxy, hetarylalkyl etc.) are optionally mono- or polysubstituted, identically or differently, by halogen, cyano, C₁-C₆-alkyl, C₃-C₆-cycloalkyl, C₁-C₆-haloalkyl, cyano-C₁-C₄-alkyl, C₁-C₆-alkoxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₁-C₄-alkylcarbonylamino, C₁-C₄-haloalkylcarbonylamino, C₁-C₆-cycloalkylcarbonylamino, C₁-C₄-alkylcarbonyl, C₁-C₄-haloalkylcarbonyl, C₃-C₆-cycloalkylcarbonyl, C₃-C₆-cycloalkyl-C₁-C₂-alkylcarbonyl, C₃-C₆-halocycloalkylcarbonyl, C₁-C₆-alkoxycarbonyl, C₁-C₆-alkoxy-C₁-C₂-alkylcarbonyl, C₁-C₆-haloalkoxycarbonyl, C₁-C₆-alkylthio, C₁-C₆-haloalkylthio, C₁-C₆-alkylsulphinyl, C₁-C₆-haloalkylsulphinyl, C₁-C₆-alkylsulphonyl, C₁-C₆-haloalkylsulphonyl, C₁-C₆-alkylaminosulphonyl, C₁-C₆-haloalkylaminosulphonyl, di(C₁-C₆)alkylaminosulphonyl, C₁-C₆-alkylaminocarbonyl, di(C₁-C₆)alkylaminocarbonyl, C₁-C₆-alkyl-carbonyloxy, C₁-C₄-haloalkylcarbonyloxy, C₁-C₄-alkoxycarbonyloxy, C₁-C₄-haloalkoxycarbonyloxy, aryl, hetaryl, aryloxy, hetaryloxy.

Very particularly preferred substituents or ranges for the radicals shown in the novel compounds of the formula (I) are elucidated below (area of preference (8)).

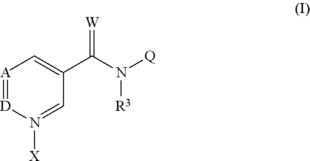

A is CR¹.

D is CR².

R¹ is hydrogen or fluorine.

R² is hydrogen.

X is a free electron pair or oxygen.

W is oxygen or sulphur.

R³ is a radical from the group of hydrogen, C₁-C₄-alkyl, C₃-C₄-cycloalkyl, C₁-C₄-haloalkyl, C₃-C₄-cycloalkyl-C₁-C₂-alkyl, C₁-C₃-alkoxy-C₁-C₂-alkyl, C₁-C₄-alkylcarbonyl, C₁-C₄-haloalkylcarbonyl, C₃-C₄-cycloalkylcarbonyl, C₁-C₃-alkoxycarbonyl, aryl, hetaryl, arylcarbonyl, hetarylcarbonyl, aryl-C₁-C₃-alkyl, hetaryl-C₁-C₃-alkyl, where the aryl, hetaryl, aryl-C₁-C₃-alkyl, hetaryl-C₁-C₃-alkyl, arylcarbonyl and hetarylcarbonyl substituents are optionally mono- or polysubstituted by halogen.

Q is Q-1

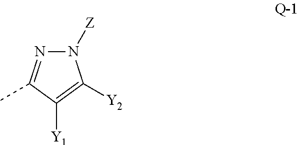

in which the dotted line is the bond to the nitrogen in the NR³ group.

Y₁ is a radical from the group of hydrogen, halogen, cyano, C₁-C₄ alkyl, C₁-C₄-haloalkyl, C₁-C₃-alkoxy, C₁-C₃-haloalkoxy.

Y₂ is a radical from the group of hydrogen, halogen, cyano, C₁-C₄-alkyl, C₃-C₅-cycloalkyl, C₁-C₄-haloalkyl, C₁-C₄-alkoxy, C₁-C₄-haloalkoxy, C₁-C₃-alkoxy-C₁-C₄-alkyl, C₁-C₃-haloalkoxy-C₁-C₄-alkyl, C₁-C₃-alkoxy-C₁-C₄-alkyloxy, C₁-C₄-alkoxycarbonyl.

Z is a radical from the group of hydrogen, L¹(=G)C*, L¹O(=G)C*, L³L⁴N(=G)C*, L¹(═O)ₙS*, L³L⁴N(═O)ₙS*, heterocyclyl, aryl, hetaryl, optionally singly or multiply, identically or differently, M²-substituted C₁-C₆-alkyl, C₃-C₆-cycloalkyl, C₂-C₆-alkenyl, C₃-C₆-alkynyl, optionally singly or multiply, identically or differently, M⁶-substituted (in the —C₁-C₄-alkyl group) L⁵(═O)ₙSL²N(=G)C—C₁-C₄-alkyl*, L¹(═O)ₙS—C₁-C₄-alkyl*.

M² is halogen, cyano, NL⁹L¹⁰, OL⁹, SL⁹, L¹(=G)C*, L³O(=G)C*, L³L⁴N(=G)C*, heterocyclyl, aryl, hetaryl, C₁-C₄-alkyl, C₁-C₄-haloalkyl, C₁-C₄-cyanoalkyl, C₃-C₄-cycloalkyl, C₃-C₄-halocycloalkyl.

G is oxygen.

n is 1 or 2.

L¹ and L⁵ are each independently heterocyclyl, aryl, hetaryl, optionally singly or multiply, identically or differently, M⁴-substituted C₁-C₄-alkyl, aryl-C₁-C₄-alkyl, hetaryl-C₁-C₄-alkyl, C₃-C₆-cycloalkyl, C₃-C₆-cycloalkyl-C₁-C₄-alkyl.

M⁴ is halogen, cyano, C₁-C₃-haloalkyl, C₁-C₃-alkoxy, C₁-C₃-haloalkoxy.

L² is hydrogen, optionally singly or multiply, identically or differently, M⁵-substituted C₁-C₄-alkyl, C₃-C₆-cycloalkyl, C₃-C₆-cycloalkyl-C₁-C₄-alkyl.

L³ and L⁴ are each independently hydrogen, heterocyclyl, aryl, hetaryl, optionally singly or multiply, identically or differently, M⁵-substituted C₁-C₄-alkyl, aryl-C₁-C₄-alkyl, hetaryl-C₁-C₄-alkyl, C₃-C₆-cycloalkyl, C₃-C₆-cycloalkyl-C₁-C₄-alkyl.

M⁵ is halogen, cyano, C₁-C₃-haloalkyl, C₁-C₃-alkoxy, C₁-C₄-alkoxycarbonyl.

M⁶ is halogen, cyano, C₁-C₃-haloalkyl.

L⁹ and L¹⁰ are each independently hydrogen, C₁-C₄-alkyl, C₁-C₄-haloalkyl, C₃-C₆-cycloalkyl, C₃-C₆-halocycloalkyl, C₃-C₆-cycloalkyl-C₁-C₄-alkyl, C₃-C₆-halocycloalkyl-C₁-C₄-alkyl, cyano-C₁-C₄-alkyl, C₁-C₄-alkoxy-C₁-C₄-alkyl, C₁-C₄-haloalkoxy-C₁-C₄-alkyl, C₁-C₄-alkylcarbonyl, C₁-C₄-haloalkylcarbonyl, C₃-C₆-cycloalkylcarbonyl, C₃-C₆-cycloalkyl-C₁-C₄-alkylcarbonyl, C₃-C₆-halocycloalkylcarbonyl, C₁-C₄-alkoxycarbonyl, C₁-C₄-alkoxy-C₁-C₄-alkylcarbonyl, C₁-C₄-haloalkoxycarbonyl, C₁-C₄-alkylaminocarbonyl, di(C₁-C₄)alkylaminocarbonyl, C₃-C₆-cycloalkylaminocarbonyl, C₃-C₆-cycloalkyl-C₁-C₄-alkylaminocarbonyl, aryl, hetaryl, heterocyclyl, aryl-C₁-C₃-alkyl, hetaryl-C₁-C₃-alkyl.

In this context, when Y₂ is methyl and Z is phenyl-C₁-C₆-alkyl, the phenyl ring in the ortho position is not substituted by alkoxy or by arylalkoxy; in addition, Z is not 3,5-dimethyl-1,2-oxazol-4-ylmethyl when A and D are each CH and, at the same time, X is a free electron pair, W is oxygen, Q is Q-1 and R³, Y₁ and Y₂ are each hydrogen; and finally, compounds of the formula (G1) are excluded.

In the very particularly preferred substituents or ranges mentioned, unless stated otherwise, the heterocyclyl, aryl and hetaryl radicals (including as part of a larger unit such as aryloxy, hetarylalkyl etc.) are optionally mono- or polysubstituted, identically or differently, by halogen, cyano, C₁-C₄-alkyl, C₃-C₆-cycloalkyl, C₁-C₄-haloalkyl, C₁-C₄-alkoxy, C₁-C₄-haloalkoxy, C₃-C₆-halocycloalkyl, C₁-C₄-alkoxy-C₁-C₄-alkyl, C₁-C₄-haloalkoxy-C₁-C₄-alkyl, C₁-C₄-alkoxycarbonyl, C₁-C₄-haloalkoxycarbonyl, C₁-C₃-alkylthio, C₁-C₃-haloalkylthio, C₁-C₃-alkylsulphonyl, C₁-C₃-haloalkylsulphonyl, C₁-C₃-alkylaminosulphonyl, C₁-C₃-haloalkylaminosulphonyl, di(C₁-C₃)alkylaminosulphonyl, C₁-C₃-alkylaminocarbonyl, di(C₁-C₃)alkylaminocarbonyl, aryl, hetaryl, aryloxy, hetaryloxy.

Explicitly very particularly preferred substituents or ranges for the radicals shown in the novel compounds of the formula (I) are elucidated below (area of preference (9)).

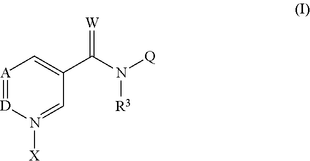

A is CR¹.

D is CR².

R¹ is hydrogen or fluorine.

R² is hydrogen.

X is a free electron pair.

W is oxygen.

R³ is a radical from the group of hydrogen, C₁-C₄-alkyl, C₁-C₄-haloalkyl, C₃-C₄-cycloalkyl-C₁-C₂-alkyl, C₁-C₄-alkylcarbonyl, arylcarbonyl, hetarylcarbonyl, where the arylcarbonyl and hetarylcarbonyl substituents are optionally mono- or polysubstituted by halogen.

Q is Q-1

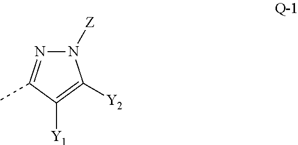

in which the dotted line is the bond to the nitrogen in the NR³ group.

Y₁ is a radical from the group of hydrogen, halogen, cyano, methyl, ethyl, n-propyl, iso-propyl, trifluoromethyl, difluoromethyl, methoxy, ethoxy, difluoromethoxy, trifluoromethoxy.

Y₂ is a radical from the group of hydrogen, halogen, cyano, C₁-C₄-alkyl, C₃-C₅-cycloalkyl, C₁-C₃-haloalkyl, C₁-C₃-alkoxy, C₁-C₃-haloalkoxy, C₁-C₃-alkoxy-C₁-C₂-alkyl, C₁-C₃-haloalkoxy-C₁-C₂-alkyl, C₁-C₃-alkoxy-C₁-C₂-alkyloxy, C₁-C₃-alkoxycarbonyl.

Z is a radical from the group of hydrogen, L¹(=G)C*, L¹O(=G)C*, L³L⁴N(=G)C*, L¹(═O)ₙS*, L³L⁴N(═O)ₙS*, aryl, hetaryl, C₃-C₆-cycloalkyl, C₃-C₆-halocycloalkyl, C₂-C₆-alkenyl, C₃-C₆-alkynyl, C₂-C₆-haloalkenyl, C₃-C₆-haloalkynyl, optionally singly or multiply, identically or differently, M²-substituted C₁-C₆-alkyl, optionally singly or multiply, identically or differently, M⁶-substituted (in the —C₁-C₄-alkyl group) by L⁵(═O)ₙSL²N(=G)C—C₁-C₄-alkyl*, L¹(═O)ₙS—C₁-C₄-alkyl*.

M² is halogen, cyano, heterocyclyl, aryl, hetaryl, C₃-C₄-cycloalkyl, C₃-C₄-halocycloalkyl, NL⁹R¹⁰, OL⁹, SL⁹, L¹(=G)C*, L³O(=G)C*, L³L⁴N(=G)C*.

G is oxygen.

n is 1 or 2.

L¹ and L⁵ are each independently aryl, hetaryl, C₁-C₄-alkyl, C₁-C₄-haloalkyl, C₁-C₄-cyanoalkyl, C₁-C₃-alkoxy-C₁-C₄-alkyl, C₁-C₃-haloalkoxy-C₁-C₄-alkyl, aryl-C₁-C₄-alkyl, aryl-C₁-C₄-haloalkyl, aryl-C₁-C₄-cyanoalkyl, hetaryl-C₁-C₄-alkyl, hetaryl-C₁-C₄-haloalkyl, hetaryl-C₁-C₄-cyanoalkyl, C₃-C₆-cycloalkyl, C₃-C₆-halocycloalkyl, C₃-C₆-cycloalkyl-C₁-C₄-alkyl, C₃-C₆-halocycloalkyl-C₁-C₄-alkyl.

L² is hydrogen or C₁-C₄-alkyl.

L³ and L⁴ are each independently hydrogen, aryl, hetaryl, C₁-C₄-alkyl, C₁-C₄-haloalkyl, C₁-C₄-cyanoalkyl, C₁-C₃-alkoxy-C₁-C₄-alkyl, aryl-C₁-C₄-alkyl, aryl-C₁-C₄-haloalkyl, aryl-C₁-C₄-cyanoalkyl, hetaryl-C₁-C₄-alkyl, hetaryl-C₁-C₄-haloalkyl, hetaryl-C₁-C₄-cyanoalkyl, C₃-C₆-cycloalkyl, C₃-C₆-halocycloalkyl, C₃-C₆-cycloalkyl-C₁-C₄-alkyl, C₃-C₆-halocycloalkyl-C₁-C₄-alkyl.

M⁶ is halogen, cyano, C₁-C₃-haloalkyl.

L⁹ and L¹⁰ are each independently hydrogen, C₁-C₄-alkyl, C₁-C₄-haloalkyl, C₃-C₆-cycloalkyl, C₃-C₆-cycloalkyl-C₁-C₄-alkyl, cyano-C₁-C₄-alkyl, C₁-C₄-alkylcarbonyl, C₁-C₄-haloalkylcarbonyl, C₃-C₆-cycloalkyl-C₁-C₄-alkylcarbonyl, C₁-C₄-alkoxycarbonyl, C₁-C₄-alkylaminocarbonyl, di(C₁-C₄)alkylaminocarbonyl, C₃-C₆-cycloalkylaminocarbonyl, C₃-C₆-cycloalkyl-C₁-C₄-alkylaminocarbonyl, aryl, hetaryl, aryl-C₁-C₃-alkyl, hetaryl-C₁-C₃-alkyl.

In this context, when Y₂ is methyl and Z is phenyl-C₁-C₆-alkyl, the phenyl ring in the ortho position is not substituted by alkoxy or by arylalkoxy; in addition, Z is not 3,5-dimethyl-1,2-oxazol-4-ylmethyl when A and D are each CH and, at the same time, X is a free electron pair, W is oxygen, Q is Q-1 and R³, Y₁ and Y₂ are each hydrogen; and finally, compounds of the formula (G1) are excluded.

In the explicitly very particularly preferred substituents or ranges mentioned, unless stated otherwise, the heterocyclyl, aryl and hetaryl radicals (including as part of a larger unit such as aryloxy, hetarylalkyl etc.) are optionally mono- or polysubstituted, identically or differently, by halogen, cyano, C₁-C₃-alkyl, C₁-C₃-haloalkyl, C₁-C₂-alkoxy, C₁-C₂-haloalkoxy, C₁-C₃-alkoxycarbonyl, C₁-C₃-haloalkoxycarbonyl, C₁-C₂-alkylthio, C₁-C₂-haloalkylthio, C₁-C₂-alkylsulphonyl, C₁-C₂-haloalkylsulphonyl, C₁-C₃-alkylaminosulphonyl, C₁-C₃-alkylaminocarbonyl, aryl, hetaryl, aryloxy, hetaryloxy.

A preferred embodiment of the invention relates to compounds of the formula (I) in which the substituents or ranges of radicals listed are defined as follows. The combination thereof forms the range of preference (10).

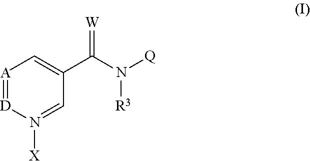

A is N or CR¹.

D is N or CR², where A and D are not both N.

R¹ is hydrogen, chlorine, bromine or fluorine.

R² is hydrogen, methyl, methoxy or chlorine.

X is a free electron pair or oxygen.

W is oxygen or sulphur.

R³ is a radical from the group of hydrogen, C₁-C₃-alkyl, C₁-C₃-haloalkyl, C₃-C₄-alkynyl, cyano-C₁-C₂-alkyl, arylcarbonyl and hetaryl-C₁-C₂-alkyl, where the arylcarbonyl and hetaryl-C₁-C₂-alkyl substituents are optionally singly or multiply substituted, identically or differently, by halogen.

Q is Q-1

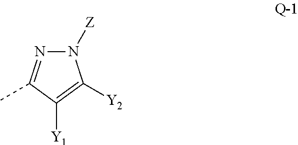

in which the dotted line is the bond to the nitrogen in the NR³ group.

Y₁ is a radical from the group of hydrogen, halogen, C₁-C₃-alkyl, cyano and C₁-C₂-alkoxycarbonyl,

Y₂ is a radical from the group of hydrogen, C₁-C₃-alkyl, C₃-C₅-cycloalkyl, C₁-C₂-haloalkyl, halogen, cyano, C₁-C₂-haloalkoxy, C₁-C₂-alkoxycarbonyl, C₁-C₂-alkylthio and C₁-C₂-alkylsulphinyl, or

Y₁ and Y₂ together with the carbon atoms to which they are bonded form a phenyl ring or a 6-membered heteroaromatic ring which is optionally mono- or polysubstituted by methyl.

Z is a radical from the group of hydrogen, C₁-C₆-alkyl, C₁-C₄-haloalkyl, C₃-C₇-cycloalkyl, C₂-C₆-alkenyl, C₂-C₄-haloalkenyl, C₃-C₅-alkynyl, cyano-C₁-C₄-alkyl, C₃-C₅-cycloalkyl-C₁-C₃-alkyl, C₃-C₅-halocycloalkyl-C₁-C₂-alkyl, aryl, hetaryl, aryl-C₁-C₂-alkyl, hetaryl-C₁-C₂-alkyl, hetaryl-C₁-C₃-haloalkyl, heterocyclyl-C₁-C₂-alkyl, C₁-C₆-alkylaminocarbonyl, C₃-C₆-cycloalkylaminocarbonyl, C₁-C₃-haloalkylaminocarbonyl, C₃-C₅-cycloalkyl-C₁-C₂-alkylaminocarbonyl, C₁-C₂-alkoxy-C₁-C₄-alkylaminocarbonyl, di(C₁-C₃-alkoxy)-C₁-C₄-alkylaminocarbonyl, C₁-C₂-haloalkoxy-C₁-C₃-alkylaminocarbonyl, di(C₁-C₃)alkylaminocarbonyl, di(C₁-C₂-alkoxy-C₁-C₄-alkyl)aminocarbonyl, aryl-C₁-C₄-alkylaminocarbonyl, hetaryl-C₁-C₄-alkylaminocarbonyl, heterocyclyl-C₁-C₃-alkylaminocarbonyl, aryloxy-C₁-C₃-alkylaminocarbonyl, C₁-C₂-alkylsulphonyl-(C₁-C₂-alkylamino)carbonyl, arylsulphonyl-(C₁-C₂-alkylamino)carbonyl, C₁-C₄-alkylaminosulphonyl, di(C₁-C₂)alkylaminosulphonyl, C₁-C₃-alkylsulphonyl, hetarylsulphonyl, heterocyclylsulphonyl, C₁-C₅-alkoxycarbonyl, C₁-C₃-haloalkoxycarbonyl, aryloxycarbonyl, C₁-C₅-alkylcarbonyl, C₃-C₅-cycloalkylcarbonyl, arylcarbonyl, hetarylcarbonyl, heterocyclylcarbonyl, C₁-C₂-alkoxy-C₁-C₃-alkyl, C₁-C₃-alkylthio-C₁-C₃-alkyl, C₁-C₃-alkylsulphinyl-C₁-C₃-alkyl, C₁-C₃-alkylsulphonyl-C₁-C₃-alkyl, hetarylthio-C₁-C₂-alkyl, hetarylsulphinyl-C₁-C₂-alkyl, hetarylsulphonyl-C₁-C₂-alkyl, C₁-C₂-alkylthio-C₁-C₃-alkylcarbonyl, C₁-C₂-alkylsulphonyl-C₁-C₃-alkylcarbonyl, C₁-C₂-alkylthio-C₁-C₃-alkylaminocarbonyl, C₁-C₂-alkylsulphinyl-C₁-C₃-alkylaminocarbonyl, C₁-C₂-alkylsulphonyl-C₁-C₃-alkylaminocarbonyl, C₁-C₂-alkoxy-C₁-C₂-alkylcarbonyl, C₃-C₅-halocycloalkylcarbonyl-C₁-C₂-alkyl, C₁-C₃-alkoxycarbonyl-C₁-C₄-alkyl, C₁-C₂-alkoxycarbonyloxy-C₁-C₃-alkyl, C₁-C₃-alkylaminocarbonyl-C₁-C₂-alkyl, hydroxycarbonyl-C₁-C₂-alkyl, C₁-C₂-alkoxycarbonyl-C₁-C₄-alkylaminocarbonyl-C₁-C₂-alkyl, C₁-C₃-alkoxycarbonyl-di-(C₁-C₂)alkylaminocarbonyl-C₁-C₂-alkyl, hetaryl-C₂-C₃-haloalkenyl and aryl-C₁-C₂-alkylcarbonyl-C₁-C₂-alkyl,

where the aryl, hetaryl, aryl-C₁-C₂-alkyl, hetaryl-C₁-C₂-alkyl, hetaryl-C₁-C₃-haloalkyl, aryl-C₁-C₄-alkylaminocarbonyl, hetaryl-C₁-C₄-alkylaminocarbonyl, aryloxy-C₁-C₃-alkylaminocarbonyl, arylsulphonyl-(C₁-C₂-alkylamino)carbonyl, hetarylsulphonyl, aryloxycarbonyl, arylcarbonyl, hetarylcarbonyl, hetarylthio-C₁-C₂-alkyl, hetarylsulphinyl-C₁-C₂-alkyl, hetarylsulphonyl-C₁-C₂-alkyl, hetaryl-C₂-C₃-haloalkenyl and aryl-C₁-C₂-alkylcarbonyl-C₁-C₂-alkyl substituents are optionally mono- or polysubstituted, identically or differently, by halogen, cyano, C₁-C₅-alkyl, C₁-C₂-haloalkyl, di(C₁-C₂)alkylaminocarbonyl, C₁-C₃-alkoxycarbonyl, phenyl, C₁-C₂-alkoxy, C₁-C₂-haloalkoxy, C₁-C₃-haloalkylthio or COOH,

and where the heterocyclyl-C₁-C₂-alkyl, heterocyclyl-C₁-C₃-alkylaminocarbonyl, heterocyclylsulphonyl and heterocyclylcarbonyl substituents are optionally mono- or polysubstituted, identically or differently, by C₁-C₃-alkyl, C₃-C₅-cycloalkyl or C₁-C₂-alkoxy,

and with the proviso that, when Y₂ is methyl and Z is aryl-C₁-C₂-alkyl, the phenyl ring in the ortho position is not substituted by alkoxy or by arylalkoxy, and with the proviso that Z is not 3,5-dimethyl-1,2-oxazol-4-ylmethyl when A and D are each CH and, at the same time, X is a free electron pair, W is oxygen and R³, Y₁ and Y₂ are each hydrogen, and with the proviso that compounds of the formula (G1) are excluded.

In the preferred definitions (range of preference (6)), unless stated otherwise:

halogen is selected from the group of fluorine, chlorine, bromine and iodine, preferably in turn from the group of fluorine, chlorine, bromine and iodine,

aryl (including as part of a larger unit, for example arylalkyl) is selected from the group of phenyl, naphthyl, anthryl, phenanthrenyl, and is preferably in turn phenyl,

hetaryl (synonymous with heteroaryl, including as part of a larger unit, for example hetarylalkyl) is selected from the group of furyl, thienyl, pyrrolyl, pyrazolyl, imidazolyl, triazolyl, oxazolyl, isoxazolyl, thiazolyl, isothiazolyl, oxadiazolyl, thiadiazolyl, tetrazolyl, pyridyl, pyrimidinyl, pyridazinyl, pyrazinyl, triazinyl, benzofuryl, benzoisofuryl, benzothienyl, benzoisothienyl, indolyl, isoindolyl, indazolyl, benzothiazolyl, benzoisothiazolyl, benzoxazolyl, benzisoxazolyl, benzimidazolyl, benzoxadiazolyl, quinolinyl, isoquinolinyl, cinnolinyl, phthalazinyl, quinazolinyl, quinoxalinyl, naphthyridinyl, benzotriazinyl, purinyl, pteridinyl, indolizinyl, imidazopyridinyl and benzothiadiazole.

Heterocyclyl is a saturated or unsaturated, non-heteroaromatic radical containing at least one 3- to 7-membered heterocyclic ring (=carbocyclic ring in which at least one carbon atom has been replaced by a heteroatom, preferably by a heteroatom from the group of N, O, S); this may be unsubstituted or substituted, in which case the bonding site is localized on a ring atom. Heterocyclyl is preferably selected from the group of aziridinyl, azirenyl, oxiranyl, oxirenyl, thiiranyl, thiirenyl, azetidinyl, dihydroazetyl, oxetanyl, oxetyl, thietanyl, thietyl, dioxetanyl, dithietanyl, oxathietanyl, pyrrolidinyl, dihydropyrrolyl, piperidinyl, tetrahydropyridinyl, dihydropyridinyl, azepanyl, tetrahydrofuranyl, dihydrofuranyl, tetrahydropyranyl, dihydropyranyl, pyranyl, oxepanyl, tetrahydrothiophenyl, dihydrothiophenyl, tetrahydrothiopyranyl, dihydrothiopyranyl, thiepanyl, dioxolanyl, dioxanyl, dioxepanyl, dithiolanyl, dithianyl, oxathiolanyl, oxathianyl, pyronyl, triazolonyl, triazolidindionyl, tetrahydropyrimidinonyl, thiadiazolopyrimidinonyl and 2-(N-cyanoimino)thiazolidinyl. If the heterocycle is a partly or fully saturated nitrogen-containing heterocycle, this may be joined to the rest of the molecule either via carbon or via the nitrogen. If sulphur occurs in rings in the above definitions, the sulphur may optionally also be in the form of SO or SO₂. Optionally, the heterocycle may be interrupted once or more than once by C═O or C═S.

In a preferred embodiment, these definitions also apply to the range of preference (10).

In the particularly preferred definitions (range of preference (7)), unless stated otherwise:

halogen selected from the group of fluorine, chlorine, bromine and iodine,

aryl (including as part of a larger unit, for example arylalkyl) is selected from the group of phenyl, naphthyl, anthryl, phenanthrenyl, and is preferably in turn phenyl,

hetaryl (synonymous with heteroaryl, including as part of a larger unit, for example hetarylalkyl) is selected from the group of furyl, thienyl, pyrrolyl, pyrazolyl, imidazolyl, triazolyl, oxazolyl, isoxazolyl, thiazolyl, isothiazolyl, oxadiazolyl, thiadiazolyl, tetrazolyl, pyridyl, pyrimidinyl, pyridazinyl, pyrazinyl, imidazopyridinyl and benzothiadiazole.

Heterocyclyl is selected from the group of aziridinyl, oxiranyl, thiiranyl, azetidinyl, oxetanyl, thietanyl, pyrrolidinyl, dihydropyrrolyl, piperidinyl, tetrahydropyridinyl, dihydropyridinyl, azepanyl, tetrahydrofuranyl, dihydrofuranyl, tetrahydropyranyl, dihydropyranyl, pyranyl, oxepanyl, tetrahydrothiophenyl, dihydrothiophenyl, tetrahydrothiopyranyl, dihydrothiopyranyl, thiepanyl, dioxolanyl, dioxanyl, dioxepanyl, dithiolanyl, dithianyl, oxathiolanyl, oxathianyl, pyronyl, triazolonyl, triazolidindionyl, tetrahydropyrimidinonyl, thiadiazolopyrimidinonyl and 2-(N-cyanoimino)thiazolidinyl.

In a preferred embodiment, these definitions also apply to the range of preference (10).

In the very particularly preferred definitions (range of preference (8)), unless stated otherwise:

halogen selected from the group of fluorine, chlorine, bromine and iodine,

aryl (including as part of a larger unit, for example arylalkyl) is selected from the group of phenyl, naphthyl, and is preferably in turn phenyl,

hetaryl (synonymous with heteroaryl, including as part of a larger unit, for example hetarylalkyl) is selected from the group of pyrazolyl, imidazolyl, triazolyl, thiazolyl, isothiazolyl, thiadiazolyl, tetrazolyl, pyridyl, pyrimidinyl, pyridazinyl, pyrazinyl, imidazopyridinyl and benzothiadiazole.

Heterocyclyl is selected from the group of aziridinyl, oxiranyl, thiiranyl, azetidinyl, oxetanyl, thietanyl, pyrrolidinyl, piperidinyl, tetrahydrofuranyl, tetrahydropyranyl, tetrahydrothiophenyl, tetrahydrothiopyranyl, dioxolanyl, dioxanyl, dithiolanyl, dithianyl, oxathiolanyl, oxathianyl, pyronyl, triazolonyl, triazolidindionyl, tetrahydropyrimidinonyl, thiadiazolopyrimidinonyl and 2-(N-cyanoimino)thiazolidinyl.

In a preferred embodiment, these definitions also apply to the range of preference (10).

In the explicitly very particularly preferred definitions (range of preference (9)), unless stated otherwise:

halogen is selected from the group of fluorine, chlorine and bromine,

aryl (including as part of a larger unit, for example arylalkyl) is phenyl,

hetaryl (synonymous with heteroaryl, including as part of a larger unit, for example hetarylalkyl) is selected from the group of pyrazolyl, imidazolyl, triazolyl, thiazolyl, isothiazolyl, thiadiazolyl, pyridyl, pyrimidinyl, pyridazinyl, pyrazinyl, imidazopyridinyl and benzothiadiazole.

Heterocyclyl is selected from the group of tetrahydrofuranyl, tetrahydropyranyl, tetrahydrothiophenyl, tetrahydrothiopyranyl, dioxolanyl, dioxanyl, dithiolanyl, dithianyl, pyronyl, triazolonyl, triazolidindionyl, tetrahydropyrimidinonyl, thiadiazolopyrimidinonyl and 2-(N-cyanoimino)thiazolidinyl.

In a preferred embodiment, these definitions also apply to the range of preference (10).

Halogen-substituted radicals, for example haloalkyl, are mono- or polyhalogenated, up to the maximum number of possible substituents. In the case of polyhalogenation, the halogen atoms may be the same or different. Halogen here is fluorine, chlorine, bromine and iodine.

Saturated or unsaturated hydrocarbyl radicals, for example alkyl or alkenyl, may each be straight-chain or branched as far as possible, including in combination with heteroatoms, as, for example, in alkoxy.

Optionally substituted radicals may be mono- or polysubstituted, where the substituents in the case of polysubstitution may be the same or different.

The radical definitions or elucidations given above in general terms or within areas of preference apply to the end products and correspondingly to the starting materials and intermediates. These radical definitions can be combined with one another as desired, i.e. including combinations between the respective preferred ranges.

Preference is given in accordance with the invention to novel compounds of the formula (I) in which a combination of the definitions listed above as preferred is present (area of preference (6)).

Particular preference is given in accordance with the invention to novel compounds of the formula (I) in which a combination of the definitions listed above as particularly preferred is present (area of preference (7)).

Very particular preference is given in accordance with the invention to novel compounds of the formula (I) in which a combination of the definitions listed above as very particularly preferred is present (area of preference (8)).

Explicitly very particular preference is given in accordance with the invention to novel compounds of the formula (I) in which a combination of the definitions listed above as explicitly very particularly preferred is present (area of preference (9)).

Further preferred embodiments of the invention relate to compounds as defined by the areas of preference (1) to (10) in which W is oxygen.

Further preferred embodiments of the invention relate to compounds as defined by the areas of preference (1) to (10) in which W is sulphur.

Further preferred embodiments of the invention relate to compounds as defined by the areas of preference (1) to (10) in which W is NR⁴.

Further preferred embodiments of the invention relate to compounds as defined by the areas of preference (1) to (10) in which X is an electron pair.

Further preferred embodiments of the invention relate to compounds as defined by the areas of preference (1) to (10) in which W is oxygen, X is an electron pair and Y₁ and Y₂ together with the atoms to which they are bonded do not form a ring.

Further preferred embodiments of the invention relate to compounds as defined by the areas of preference (1) to (10) in which W is oxygen, X is an electron pair and Y₁ and Y₂ together with the atoms to which they are bonded do not form a ring, and A and D are each C—H or C-halogen.

Further preferred embodiments of the invention relate to compounds as defined by the areas of preference (1) to (10) in which W is oxygen, X is an electron pair and Y₁ and Y₂ together with the atoms to which they are bonded form a ring (preferably phenyl or optionally methyl-substituted pyridyl), and A and D are each C—H or C-halogen.

Further preferred embodiments of the invention relate to compounds as defined by the areas of preference (1) to (10) in which W is oxygen, X is an electron pair and Y₁ and Y₂ together with the atoms to which they are bonded do not form a ring, and A and D are each C—H.

Further preferred embodiments of the invention relate to compounds as defined by the areas of preference (1) to (10) in which W is oxygen, X is an electron pair and Y₁ and Y₂ together with the atoms to which they are bonded do not form a ring, and A and D are each C-halogen.

The novel compounds of the formula (I) can be prepared by processes described hereinafter.

The invention also relates to processes for preparing the novel compounds of the formula (I) in which W is O

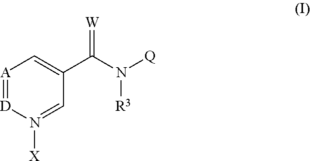

where A, D, X, R³ and Q are each defined as described above, by reacting amines of the formulae (II-1) to (II-4)

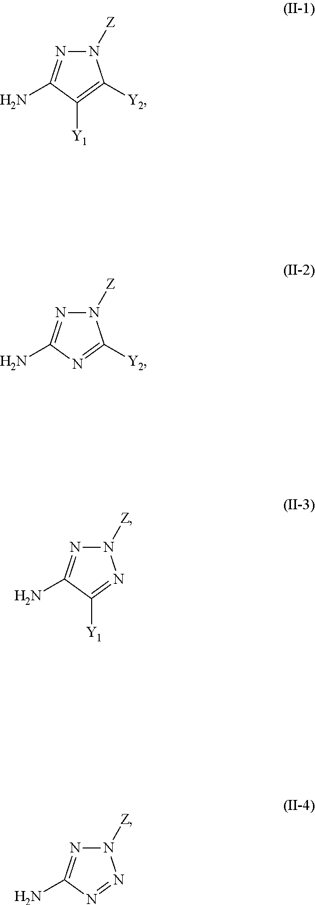

in which Z, Y₁ and Y₂ are each defined as described above,

with carboxylic acids or carboxylic acid derivatives of the formula (III)

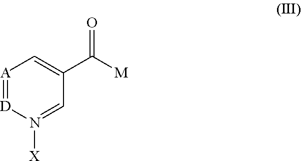

in which
M is halogen, hydroxyl, alkoxy, alkylsulphanyl, acyloxy, sulphonyloxy, azide or N-heterocyclyl (e.g. imidazolyl),
A is as defined above,
D is as defined above and
X is as defined above.

The process is illustrated in Synthesis Scheme 1.

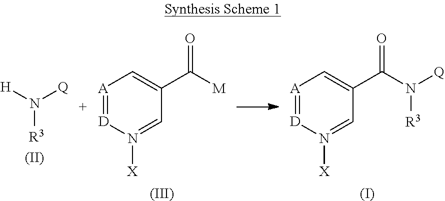

In this case, compounds of the formula (III) may already have been activated or may be activated in situ. For example, it is possible to use compounds of the formula (III) as acid halides (e.g. M=chlorine). In that case, the reaction is advantageously conducted in the presence of a base, for example triethylamine, pyridine or sodium hydroxide, or of another reagent, for example silver cyanide. Alternatively, it is possible to use carboxylic acids (M=OH) in the presence of coupling reagents, for example dicyclohexylcarbodiimide, and additives, for example 1-hydroxy-1H-benzotriazole (W. Konig, R. Geiger, Chem. Ber. 1970, 103, 788). It is also possible to use coupling reagents such as 1-ethyl-3-(3-dimethylaminopropyl)carbodiimide, 1,1′-carbonyldiimidazole, O-(benzotriazol-1-tetramethyluronium hexafluorophosphate, O-(7-azabenzotriazol-1-yl)-N,N,N′,N′-tetramethyluronium hexafluorophosphate, propanephosphonic cyclic anhydride and similar compounds. Suitable coupling reagents for performance of the preparation process are in principle all compounds that enable the formation of an amide bond (cf., for example, E. Valeur, M. Bradley, Chem. Soc. Rev. 2009, 38, 606; S.-Y. Han, Y.-A. Kim, Tetrahedron 2004, 60, 2447). It is also possible to use additives such as 1-hydroxy-7-azabenzotriazole, 4-(dimethylamino)pyridine, diisopropylethylamine and similar compounds. In addition, it is also possible to use symmetric or mixed anhydrides for preparation of compounds of the formula (I) (G. W. Anderson, J. E. Zimmerman, F. M. Calahan, J. Am. Chem. Soc. 1967, 89, 5012). It is possible here to use various chloroformic esters, for example iso-butyl chloroformate and sec-butyl chloroformate. It is likewise possible to use, for example, isovaleryl chloride and pivaloyl chloride.

Compounds of the formula (I) in which W is O and X is a free electron pair and R³ is different from hydrogen can likewise be prepared by alkylation according to Synthesis Scheme 2 from compounds of the formula (I) in which R³ is hydrogen. It is possible here to use suitable alkylating agents, for example alkyl halides or acyl halides, in the presence of particular bases, for example diisopropylethylamine, triethylamine, pyridine or caesium carbonate.

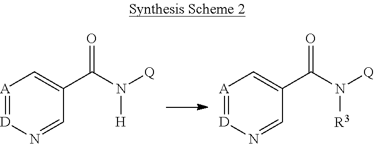

Compounds of the formula (I) in which Z is O (oxygen atom) and X is a free electron pair can be reacted according to Synthesis Scheme 3 with a thionating agent, for example diphosphorus pentasulphide or Lawesson's reagent (cf., for example, C. P. Dell in Comprehensive Organic Functional Group Transformations, vol. 5, eds.: A. R. Katrizky, O. Meth-Cohn, C. W. Rees, Pergamon, Oxford, 1995, p. 565-628; M. Jesberger, T. P. Davis, L. Barner, Synthesis 2003, 13, 1929), to give compounds of the formula (I) in which Z is S (sulphur atom).

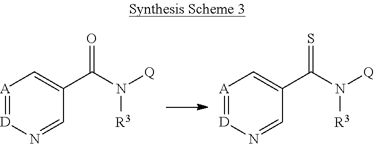

Compounds of the formula (I) in which Z is S and X is a free electron pair can be converted according to Synthesis Scheme 4 to compounds of the formula (I) in which Z is NR⁴, by reaction with suitable amines (V. A. Glushkov, L. V. Anikina, Yu. B. Vikharev, E. V. Feshina, Yu. V. Shklyaev, Pharmaceutical Chemistry Journal 2005, 39, 533). Alternatively, compounds of the formula (I) in which Z is O and X is a free electron pair can be activated according to Synthesis Scheme 5, for example via chlorination with thionyl chloride, phosphorus oxychloride or phosphorus pentachloride, and then converted with suitable amines to compounds of the formula (I) in which Z is NR⁴.

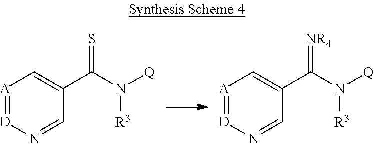

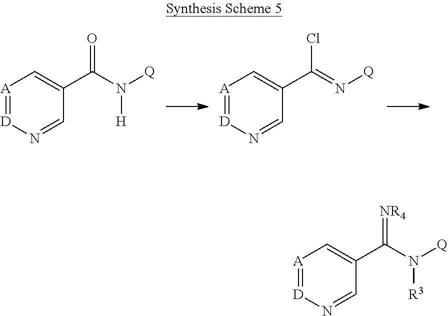

The pyridine-3-carboxylic acids, pyrimidine-5-carboxylic acids and pyridazine-5-carboxylic acids required for the preparation of the inventive compounds of the formula (I) are commercially available or can be synthesized by methods known from the literature.

### Certain Amines of the Formula (II-1)

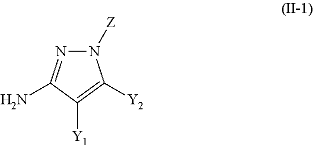

are commercially available or known from the literature, or can be synthesized by processes known from the literature. These are mentioned by way of example below.

| Amines of the formula (II-1) | Comment | Amines of the formula (II-1) | Comment |
|---|---|---|---|
| 1-(2,2-difluoroethyl)-5- | commercially | 1-(2,4-difluorophenyl)-1H- | commercially |
| methyl-1H-pyrazole-3-amine | available | pyrazole-3-amine | available |
| 1,5-dimethyl-1H-pyrazole-3- | commercially | 1-(2,4-dichlorobenzyl)-1H- | commercially |
| amine | available | pyrazole-3-amine | available |
| 1-(pyrimidin-2-ylmethyl)-1H- | commercially | 1-(2,5-difluorobenzyl)-1H- | commercially |
| pyrazole-3-amine | available | pyrazole-3-amine | available |
| 1-(2,2,2-trifluoroethyl)-1H- | commercially | 2-[(3-amino-1H-pyrazol-1- | commercially |
| pyrazole-3-amine | available | yl)methyl]benzonitrile | available |
| 1-isobutyl-5-methyl-1H- | commercially | 1-[2-(trifluoromethyl)benzyl]-1H- | commercially |
| pyrazole-3-amine | available | pyrazole-3-amine | available |
| 1-(2,2-difluoroethyl)-1H- | commercially | 1-(2-fluorobenzyl)-1H-pyrazole-3- | commercially |
| pyrazole-3-amine | available | amine | available |
| N,1,5-trimethyl-1H-pyrazole- | commercially | 1-benzyl-1H-pyrazole-3-amine | commercially |
| 3-amine | available | | available |
| 5-methyl-1H-pyrazole-3- | commercially | 1-(2,4-difluorophenyl)-5-ethyl- | commercially |
| amine | available | 1H-pyrazole-3-amine | available |
| tert-butyl 3-amino-5-methyl- | commercially | 5-methyl-1-[3- | commercially |
| 1H-pyrazole-1-carboxylate | available | (trifluoromethyl)benzyl]-1H- | available |
| | | pyrazole-3-amine | |
| 5-methyl-1-(pyrimidin-2-yl)- | commercially | 1-(3,4-dichlorophenyl)-5-methyl- | commercially |
| 1H-pyrazole-3-amine | available | 1H-pyrazole-3-amine | available |
| ethyl (3-amino-5-methyl-1H- | commercially | 5-(difluoromethoxy)-1-methyl-1H- | commercially |
| pyrazol-1-yl)acetate | available | pyrazole-3-amine | available |
| 1-(pyridin-2-ylmethyl)-1H- | commercially | 1-(3,4-dichlorophenyl)-5-ethyl- | commercially |
| pyrazole-3-amine | available | 1H-pyrazole-3-amine | available |
| 1-(3-fluoropyridin-2-yl)-1H- | commercially | 5-ethyl-1-(3-methylphenyl)-1H- | commercially |
| pyrazole-3-amine | available | pyrazole-3-amine | available |
| 1-benzyl-5-ethyl-1H- | commercially | 1-(3-chlorophenyl)-5-ethyl-1H- | commercially |
| pyrazole-3-amine | available | pyrazole-3-amine | available |
| 1-ethyl-5-methyl-1H- | commercially | 4-(3-amino-5-ethyl-1H-pyrazol-1- | commercially |
| pyrazole-3-amine | available | yl)benzonitril | available |
| 1-[3-chloro-5- | commercially | 1-(4-chlorophenyl)-5-ethyl-1H- | commercially |
| (trifluoromethyl)pyridin-2- | available | pyrazole-3-amine | available |
| yl]-5-methyl-1H-pyrazole-3- | | | |
| amine | | | |
| 5-methyl-1-[5- | commercially | 5-ethyl-1-phenyl-1H-pyrazole-3- | commercially |
| (trifluoromethyl)pyridin-2- | available | amine | available |
| yl]-1H-pyrazole-3-amine | | | |
| 1-benzyl-5-methyl-1H- | commercially | 5-methyl-1-(3-methylphenyl)-1H- | commercially |
| pyrazole-3-amine | available | pyrazole-3-amine | available |
| 1-(3-chloropyridin-2-yl)-1H- | commercially | 1-(3-chlorophenyl)-5-methyl-1H- | commercially |
| pyrazole-3-amine | available | pyrazole-3-amine | available |
| 1-isopropyl-5-methyl-1H- | commercially | 1-(4-chlorophenyl)-5-methyl-1H- | commercially |
| pyrazole-3-amine | available | pyrazole-3-amine | available |
| 5-methyl-1-(3-methylbenzyl)- | commercially | 5-methyl-1-phenyl-1H-pyrazole-3- | commercially |
| 1H-pyrazole-3-amine | available | amine | available |
| 1-(pyridin-2-yl)-1H-pyrazole- | commercially | (3-amino-5-methyl-1H-pyrazol-1- | commercially |
| 3-amine | available | yl)acetic acid | available |
| 1-(3-fluorobenzyl)-5-methyl- | commercially | 4-chloro-1-(2-chlor-6- | commercially |
| 1H-pyrazole-3-amine | available | fluorbenzyl)-1H-pyrazole-3-amine | available |
| 1-(4-fluorobenzyl)-5-methyl- | commercially | 2-(3-amino-1H-pyrazol-1- | commercially |
| 1H-pyrazole-3-amine | available | yl)benzonitrile | available |
| 1-[3-chloro-5- | commercially | 1-(3-chlorophenyl)-1H-pyrazole- | commercially |
| (trifluoromethyl)pyridin-2- | available | 3-amine | available |
| yl]-1H-pyrazole-3-amine | | | |
| 1-(2-fluorobenzyl)-5-methyl- | commercially | 1-(4-methoxyphenyl)-1H- | commercially |
| 1H-pyrazole-3-amine | available | pyrazole-3-amine | available |
| 5-methoxy-1-methyl-1H- | commercially | 1-phenyl-1H-pyrazole-3-amine | commercially |
| pyrazole-3-amine | available | | available |
| methyl 4-(3-amino-5-methyl- | commercially | methyl 3-amino-1-methyl-1H- | commercially |
| 1H-pyrazol-1-yl)butanoate | available | pyrazole-5-carboxylate | available |
| 1-(2,6-difluorobenzyl)-1H- | commercially | 1-(4,6-dimethylpyrimidin-2-yl)-5- | commercially |
| pyrazole-3-amine | available | methyl-1H-pyrazole-3-amine | available |
| ethyl (3-amino-1H-pyrazol-1- | commercially | 2-(3-amino-5-methyl-1H-pyrazol- | commercially |
| yl)acetate | available | 1-yl)-N-methylacetamide | available |
| 1-(3-fluorobenzyl)-1H- | commercially | 5-methyl-1-(2,2,2-trifluoroethyl)- | commercially |
| pyrazole-3-amine | available | 1H-pyrazole-3-amine | available |
| 1-[3-(trifluoromethyl)benzyl]- | commercially | 4-methyl-1-(pyrimidin-2- | commercially |
| 1H-pyrazole-3-amine | available | ylmethyl)-1H-pyrazole-3-amine | available |
| 1-(3-methylbenzyl)-1H- | commercially | 4-chloro-1-(pyrimidin-2- | commercially |
| pyrazole-3-amine | available | ylmethyl)-1H-pyrazole-3-amine | available |
| 1-(3-methoxybenzyl)-1H- | commercially | 5-methyl-1-(pyridin-2-ylmethyl)- | WO 2009/46802 |
| pyrazole-3-amine | available | 1H-pyrazole-3-amine | |
| 1-(2,4-difluorobenzyl)-1H- | commercially | (3-amino-5-methyl-1H-pyrazol-1- | no reference |
| pyrazole-3-amine | available | yl)acetonitrile | |
| 1-(4-tert-butylbenzyl)-1H- | commercially | methyl 3-[(3-amino-1H-pyrazol-1- | US 2008/21032 |
| pyrazole-3-amine | available | yl)methyl]benzoate | |
| 1-(4-methoxybenzyl)-1H- | commercially | ethyl 4-[(3-amino-1H-pyrazol-1- | WO 2011095997 |
| pyrazole-3-amine | available | yl)methyl]benzoate | |
| 1-(2,5-dimethylbenzyl)-1H- | commercially | 1-methyl-1H-pyrazolo[3,4- | commercially |
| pyrazole-3-amine | available | b]pyridin-3-amine | available |
| 5-methyl-1-(tetrahydrofuran- | commercially | 5-methyl-1-[(4-methyl-1H- | commercially |
| 2-ylmethyl)-1H-pyrazole-3- | available | pyrazol-1-yl)methyl]-1H-pyrazole- | available |
| amine | | 3-amine | |
| 1-(pyrimidin-2-ylmethyl)-1H- | commercially | 1-tert-butyl-5-methyl-1H- | commercially |
| indazole-3-amine | available | pyrazole-3-amine | available |
| 3-(3-amino-5-methyl-1H- | commercially | 5-methyl-1-(1H-pyrazol-1- | commercially |
| pyrazol-1-yl)benzonitrile | available | ylmethyl)-1H-pyrazole-3-amine | available |

Novel 1H-pyrazole-3-amines of the formula (II-1) can generally be prepared according to Synthesis Scheme 6. The invention accordingly also relates to novel compounds of the formula (II-1) which serve as intermediates for preparation of compounds of the formula (I).

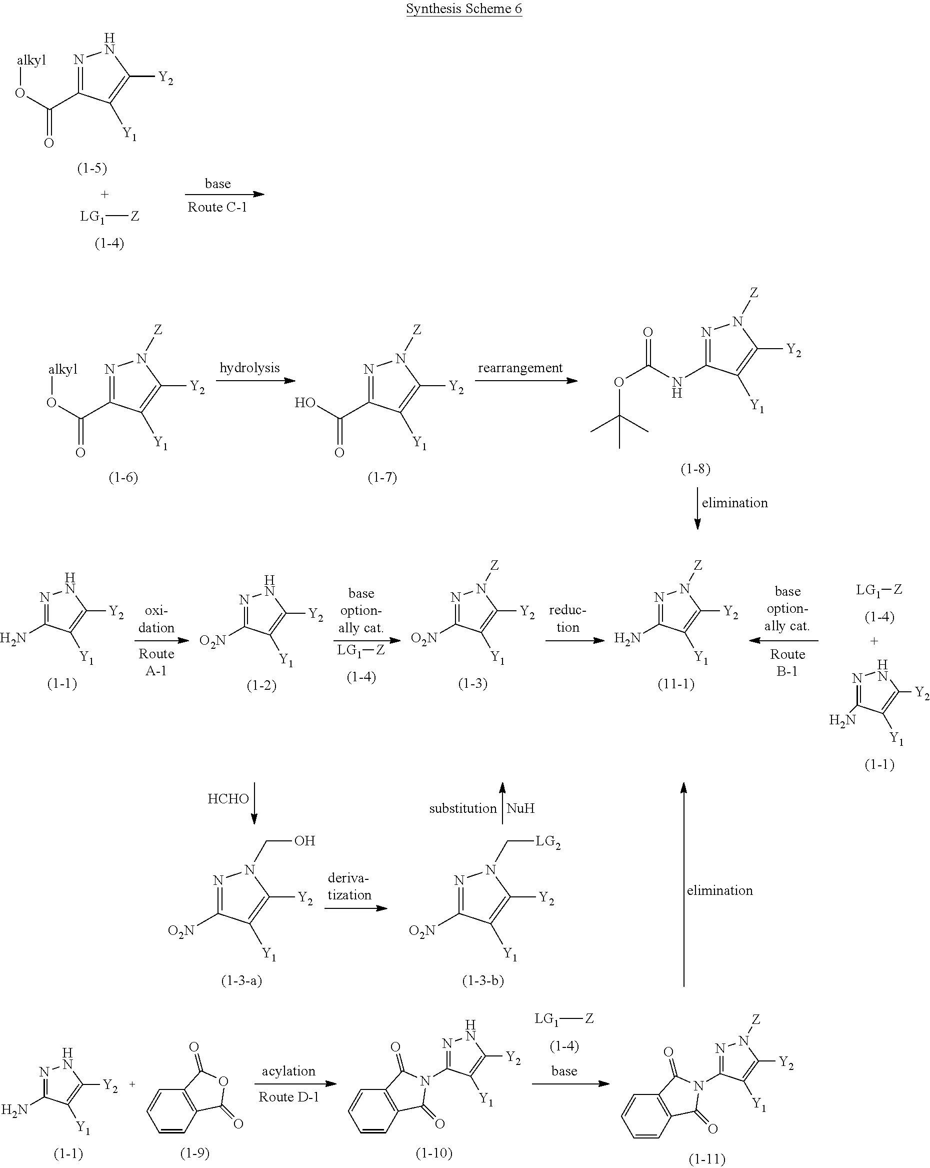

By Route A-1, it is possible to convert 1H-pyrazole-3-amines of the formula (1-1) to the corresponding 3-nitro-1H-pyrazoles of the formula (1-2) with the aid of a suitable oxidizing agent (e.g. potassium peroxymonosulphate or hydrogen peroxide (cf. M. D. Coburn, J. Het. Chem. 1970, 7, 455)). Subsequently, the reaction with compounds of the formula (1-4) having a suitable LG1 group is possible, which can be effected, for example, in the presence of a base (e.g. potassium carbonate when, for example, LG1=chlorine (cf. WO 2008/156757), pyridine for, for example LG1=Cl (cf. C. Nyffenegger et al., Synlett 2009, 8, 1318)) or sodium hydride when, for example, LG1=chlorine or bromine (cf. US 2008/0021032)), and/or of a catalyst (e.g. CuI/proline when, for example, LG1=bromine (cf. WO 2012/006475)). Alternatively, the compounds of the formula (1-2) can be reacted with formaldehyde to give (1-3-a), in which case subsequent introduction of a leaving group is possible via the hydroxyl group, for example with thionyl chloride for LG2=Cl (cf. S. Julia, Heterocycles 1986, 24, 2233). Substances of the formula (1-3-b) allow access to numerous compounds of the formula (1-3) through reaction with any kind of nucleophile NuH, which can be effected, for example, in the presence of a base such as potassium carbonate when, for example, LG1=chlorine (cf. X. Wen, Bioorg. Med. Chem. Lett. 2007, 17, 5777) or sodium hydride for, for example LG1=chlorine (cf. R. Wang, Eur. J. Org. Chem. 2007, 4, 655). Subsequent reduction of compounds of the formula (1-3) with an appropriate reducing agent (e.g. Pd/C and hydrogen (cf. US 2008/0021032), zinc dust and ammonium chloride (cf. US 2012/0010235), tin(IV) chloride and ammonium chloride (cf. WO 2011/140488) or tin(II) chloride (cf. US 2010/0310493)) enables preparation of compounds of the formula (II-1).

In an alternative route B-1, it is possible to react 1H-pyrazole-3-amines of the formula (1-1) with compounds of the formula (1-4) having a suitable LG1 group. This can be effected, for example, in the presence of a base (e.g. potassium tert-butoxide when, for example, LG1=chlorine (cf. WO 2009/012482), caesium carbonate when, for example, LG1=chlorine (cf. WO 2007/056155) or triethylamine when, for example, LG1=tert-butyloxycarbonyloxy (cf. T. Honma et al., J. Med. Chem. 2001, 44, 4628)) or/and of a coupling reagent (e.g. 1-ethyl-3-(3-dimethylaminopropyl)carbodiimide (E. Valeur, M. Bradley, Chem. Soc. Rev. 2009, 38, 606; S.-Y. Han, Y.-A. Kim, Tetrahedron 2004, 60, 2447)) and/or of a catalyst (e.g. CuI/N,N-dimethylethane-1,2-diamine when, for example, LG1=iodine (cf. WO 2008/153042) or Pd2(dba)3/Xantphos when, for example, LG1=chlorine (cf. Z. Shen, Y. Hong, X. He, W. Mo, B. Hu, N. Sun, X. Hu, Org. Lett. 2010, 12, 552)), thus enabling the formation of compounds of the formula (II-1).

According to alternative route C-1, it is possible to react 1H-pyrazole-3-carboxylic acid alkyl esters of the formula (1-5) with compounds of the formula (1-4) having a suitable LG1 group, by the methods described in Route A-1 and B-1. Subsequently, the ester can be hydrolysed, for example by suitable bases (e.g. sodium hydroxide (cf. WO 2011/063501) or lithium hydroxide (cf. D. J. Jeon et al., Bull. Korean Chem. Soc. 1998, 19, 725)) or acids (e.g. sulphuric acid (cf. WO 2008/064265)), and the resulting compounds of the formula (1-7) can be subjected to a rearrangement. This can be effected in the presence of particular reagents, for example diphenyl phosphorazidate/triethylamine/tert-butanol (cf. WO 2006/114313). The subsequent elimination of the tert-butyloxycarbonyl group under acidic conditions (e.g. dilute hydrochloric acid (cf. WO 2006/114313) or trifluoroacetic acid (cf. WO 2007/129052)) gives access to compounds of the formula (II-1).

Via Route D-1, it is possible to convert 1H-pyrazole-3-amines of the formula (1-1) to compounds of the formula (II-1) via a sequence composed of formation of the N-substituted phthalimides of the formula (1-10) by reaction with phthalic anhydride (cf. WO 2008/074832, WO 2009/106209), subsequent reaction of the products with compounds of the formula (1-4) having a suitable LG1 group, by the methods described in Route A-1 and B-1, and cleavage of the phthalimides by means of suitable reagents (e.g. hydrazine hydrate (cf. WO 2009/074677) or 2-aminoethanol (cf. M. H. Norman, D. J. Minick, G. C. Rigdon, J. Med. Chem. 1996, 39, 149)).

### Certain Amines of the Formula (II-2)

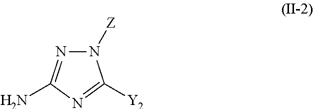

are commercially available or known from the literature, or can be synthesized by processes known from the literature. Examples include:
1H-1,2,4-triazole-3-amine (commercially available),
1-phenyl-1H-1,2,4-triazole-3,5-diamine (commercially available),
5-isopropyl-1H-1,2,4-triazole-3-amine (commercially available),
5-methyl-1H-1,2,4-triazole-3-amine (commercially available),
5-methoxy-1-methyl-1H-1,2,4-triazole-3-amine (commercially available),
1-(methoxymethyl)-1H-1,2,4-triazole-3-amine (commercially available),
1-isopropyl-5-methyl-1H-1,2,4-triazole-3-amine (commercially available),
1-(2-methylbenzyl)-1H-1,2,4-triazole-3-amine (commercially available),
5-methoxy-1-phenyl-1H-1,2,4-triazole-3-amine (commercially available),
1-(4,6-dimethylpyrimidin-2-yl)-1H-1,2,4-triazole-3-amine (commercially available),
5-chloro-1-phenyl-1H-1,2,4-triazole-3-amine (commercially available),
1-(4,5,6-trimethylpyrimidin-2-yl)-1H-1,2,4-triazole-3-amine (commercially available),
5-(methylsulphanyl)-1-phenyl-1H-1,2,4-triazole-3-amine (commercially available),
1-(3-chlorobenzyl)-1H-1,2,4-triazole-3-amine (commercially available),
1-(3-bromopyridin-2-yl)-1H-1,2,4-triazole-3-amine (commercially available),
(3-amino-1H-1,2,4-triazol-1-yl)(2-chlorophenyl)methanone (HU 39969 A2)
3-amino-N-benzyl-1H-1,2,4-triazole-1-carboxamide (S. B. Naik, V. Joshi, Indian J. Het. Chem. 2002, 12, 109).

### Certain Amines of the Formula (II-3)

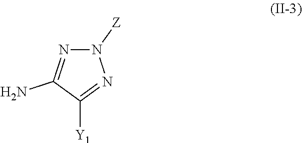

are commercially available or known from the literature, or can be synthesized by processes known from the literature. Examples include:
methyl 5-amino-2H-1,2,3-triazole-4-carboxylate (commercially available),
2-propyl-2H-1,2,3-triazole-4-amine (commercially available),
5-amino-2-isopropyl-2H-1,2,3-triazole-4-carbonitrile (commercially available),
4-(4-amino-2H-1,2,3-triazol-2-yl)butanonitrile (commercially available),
2-{[2-(methoxymethyl)-1,3-thiazol-5-yl]methyl}-2H-1,2,3-triazole-4-amine (WO 2012/077049 A1),
1-[5-amino-2-(2-chlorophenyl)-2H-1,2,3-triazol-4-yl]propan-1-one (S. I. Aziz, H. F. Anwar, D. H. Fleita, M. H. Elnagdi, J. Het. Chem. 2007, 44, 725).

### Certain Amines of the Formula (II-4)

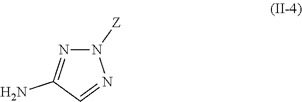

are commercially available or known from the literature, or can be synthesized by processes known from the literature. Examples include:
2H-tetrazole-5-amine (commercially available),
2-ethyl-2H-tetrazole-5-amine (commercially available),
2-allyl-2H-tetrazole-5-amine (commercially available),
3-(5-amino-2H-tetrazol-2-yl)propanonitrile (commercially available),
1-(5-amino-2H-tetrazol-2-yl)acetone (commercially available),
methyl (5-amino-2H-tetrazol-2-yl)acetate (commercially available),
5-amino-N-methyl-2H-tetrazole-2-carbothioamide (commercially available),
2-phenyl-2H-tetrazole-5-amine (commercially available),
ethyl (5-amino-2H-tetrazol-2-yl)acetate (commercially available),
2-benzyl-2H-tetrazole-5-amine (commercially available),
2-(5-amino-2H-tetrazol-2-yl)-N-(1-naphthylmethyl)acetamide (commercially available),
2-(5-amino-2H-tetrazol-2-yl)-N-butylacetamide (commercially available),
2-[(3,5-dimethyl-1,2-oxazol-4-yl)methyl]-2H-tetrazole-5-amine (US 2010/0254916),
2-(5-amino-2H-tetrazol-2-yl)-1-(2-chlorophenyl)ethyl carbamate (WO 2006/112685),
2-(3,4-dichlorobenzyl)-2H-tetrazole-5-amine (W. Holzer, B. Brandstatter, C. Jager, M. Kaun, T. Langer, W. D. Bowen, Scientia Pharmaceutica 2004, 72, 197),
2-(cyclohexylmethyl)-2H-tetrazole-5-amine (G. Quelever, S. Burlet, C. Garino, N. Pietrancosta, Y. Laras, J.-L. Kraus, J. Combinatorial Chem. 2004, 6, 695),
5-[(5-amino-2H-tetrazol-2-yl)methyl]-4-(hydroxymethyl)-2-methylpyridin-3-ol (WO 2002/004421).

In general, compounds of the formula (I) and (II) can be prepared by the processes described above. If individual compounds cannot be prepared by the processes described above, synthesis is possible by derivatization of the compounds of the formulae (I) and (II), or by individual modifications to the processes described. For example, it may have certain advantages to prepare compounds of the formula (I) or (II) from other compounds of the formulae (I) and (II), for example by hydrolysis, esterification, amide formation, reduction, etherification, oxidation, olefination, halogenation, acylation, alkylation and the like.

The processes according to the invention for preparation of the novel compounds of the formula (I) are preferably performed using a diluent. Useful diluents for performance of the processes according to the invention are, as well as water, all inert solvents. Examples include: halohydrocarbons (for example chlorohydrocarbons such as tetrachloroethylene, tetrachloroethane, dichloropropane, methylene chloride, dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, chlorotoluene, trichlorobenzene), alcohols (for example methanol, ethanol, isopropanol, butanol), ethers (for example ethyl propyl ether, methyl tert-butyl ether, anisole, phenetole, cyclohexyl methyl ether, dimethyl ether, diethyl ether, dipropyl ether, diisopropyl ether, di-n-butyl ether, diisobutyl ether, diisoamyl ether, ethylene glycol dimethyl ether, tetrahydrofuran, 1,4-dioxane, dichlorodiethyl ether and polyethers of ethylene oxide and/or propylene oxide), amines (for example trimethyl-, triethyl-, tripropyl-, tributylamine, N-methylmorpholine, pyridine and tetramethylenediamine), nitrohydrocarbons (for example nitromethane, nitroethane, nitropropane, nitrobenzene, chloronitrobenzene, o-nitrotoluene); nitriles (for example acetonitrile, propionitrile, butyronitrile, isobutyronitrile, benzonitrile, m-chlorobenzonitrile), tetrahydrothiophene dioxide, dimethyl sulphoxide, tetramethylene sulphoxide, dipropyl sulphoxide, benzyl methyl sulphoxide, diisobutyl sulphoxide, dibutyl sulphoxide, diisoamyl sulphoxide, sulphones (for example dimethyl, diethyl, dipropyl, dibutyl, diphenyl, dihexyl, methyl ethyl, ethyl propyl, ethyl isobutyl and pentamethylene sulphone), aliphatic, cycloaliphatic or aromatic hydrocarbons (for example pentane, hexane, heptane, octane, nonane and technical hydrocarbons), and also what are called “white spirits” with components having boiling points in the range from, for example, 40° C. to 250° C., cymene, petroleum fractions within a boiling range from 70° C. to 190° C., cyclohexane, methylcyclohexane, petroleum ether, ligroin, benzene, toluene, xylene, esters (for example methyl, ethyl, butyl and isobutyl acetate, dimethyl, dibutyl and ethylene carbonate); amides (for example hexamethylenephosphoramide, formamide, N-methylformamide, N,N-dimethylformamide, N,N-dipropylformamide, N,N-dibutylformamide, N-methylpyrrolidine, N-methylcaprolactam, 1,3-dimethyl-3,4,5,6-tetrahydro-2(1H)-pyrimidine, octylpyrrolidone, octylcaprolactam, 1,3-dimethyl-2-imidazolinedione, N-formylpiperidine, N,N′-diformylpiperazine) and ketones (for example acetone, acetophenone, methyl ethyl ketone, methyl butyl ketone).

It is of course also possible to perform the process according to the invention in mixtures of the solvents and diluents mentioned.

When performing the process according to the invention, the reaction temperatures can be varied within a relatively wide range. In general, the temperatures employed are between −30° C. and +150° C., preferably between −10° C. and +120° C.

The process according to the invention is generally performed under atmospheric pressure. However, it is also possible to perform the process according to the invention under elevated or reduced pressure—generally at absolute pressures between 0.1 bar and 15 bar.

To perform the process according to the invention, the starting materials are generally used in approximately equimolar amounts. However, it is also possible to use one of the components in a relatively large excess. The reaction is generally carried out in a suitable diluent in the presence of a reaction auxiliary, optionally also under a protective gas atmosphere (for example under nitrogen, argon or helium) and the reaction mixture is generally stirred at the temperature required for several hours. The workup is performed by customary methods (cf. Preparation Examples).

The basic reaction auxiliaries used to perform the processes according to the invention may be all suitable acid binders. Examples include: alkaline earth metal or alkali metal compounds (e.g. hydroxides, hydrides, oxides and carbonates of lithium, sodium, potassium, magnesium, calcium and barium), amidine bases or guanidine bases (e.g. 7-methyl-1,5,7-triazabicyclo[4.4.0]dec-5-ene (MTBD); diazabicyclo[4.3.0]nonene (DBN), diazabicyclo[2.2.2]octane (DABCO), 1,8-diazabicyclo[5.4.0]undecene (DBU), cyclohexyltetrabutylguanidine (CyTBG), cyclohexyltetramethylguanidine (CyTMG), N,N,N,N-tetramethyl-1,8-naphthalenediamine, pentamethylpiperidine) and amines, especially tertiary amines (e.g. triethylamine, trimethylamine, tribenzylamine, triisopropylamine, tributylamine, tricyclohexylamine, triamylamine, trihexylamine, N,N-dimethylaniline, N,N-dimethyltoluidine, N,N-dimethyl-p-aminopyridine, N-methylpyrrolidine, N-methylpiperidine, N-methylimidazole, N-methylpyrazole, N-methylmorpholine, N-methylhexamethylenediamine, pyridine, 4-pyrrolidinopyridine, 4-dimethylaminopyridine, quinoline, 2-picoline, 3-picoline, pyrimidine, acridine, N,N,N′,N′-tetramethylenediamine, N,N,N′,N′-tetraethylenediamine, quinoxaline, N-propyldiisopropylamine, N-ethyldiisopropylamine, N,N′-dimethylcyclohexylamine, 2,6-lutidine, 2,4-lutidine or triethyldiamine).

The acidic reaction auxiliaries used to perform the process according to the invention include all mineral acids (e.g. hydrohalic acids such as hydrofluoric acid, hydrochloric acid, hydrobromic acid or hydriodic acid, and also sulphuric acid, phosphoric acid, phosphorous acid, nitric acid), Lewis acids (e.g. aluminium(III) chloride, boron trifluoride or its etherate, titanium(IV) chloride, tin(IV) chloride) and organic acids (e.g. formic acid, acetic acid, propionic acid, malonic acid, lactic acid, oxalic acid, fumaric acid, adipic acid, stearic acid, tartaric acid, oleic acid, methanesulphonic acid, benzoic acid, benzenesulphonic acid or para-toluenesulphonic acid).

### Methods and Uses

The invention also relates to methods for controlling animal pests, in which compounds of the formula (I) are allowed to act on animal pests and/or their habitat. The control of the animal pests is preferably conducted in agriculture and forestry, and in material protection. Preferably excluded from this are methods for the surgical or therapeutic treatment of the human or animal body and diagnostic methods carried out on the human or animal body.

The invention further relates to the use of the compounds of the formula (I) as pesticides, especially crop protection agents.

In the context of the present application, the term “pesticide” in each case also always comprises the term “crop protection agent”.

The compounds of the formula (I), given good plant tolerance, favourable homeotherm toxicity and good environmental compatibility, are suitable for protecting plants and plant organs against biotic and abiotic stress factors, for increasing harvest yields, for improving the quality of the harvested material and for controlling animal pests, especially insects, arachnids, helminths, nematodes and molluscs, which are encountered in agriculture, in horticulture, in animal husbandry, in aquatic cultures, in forests, in gardens and leisure facilities, in the protection of stored products and of materials, and in the hygiene sector. They can preferably be used as pesticides. They are active against normally sensitive and resistant species and against all or some stages of development. The abovementioned pests include:

pests from the phylum of the Arthropoda, especially from the class of the Arachnida, for example Acarus spp., for example Acarus siro, Aceria kuko, Aceria sheldoni, Aculops spp., Aculus spp., for example Aculus fockeui, Aculus schlechtendali, Amblyomma spp., Amphitetranychus viennensis, Argas spp., Boophilus spp., Brevipalpus spp., for example Brevipalpus phoenicis, Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., for example Eotetranychus hicoriae, Epitrimerus pyri, Eutetranychus spp., for example Eutetranychus banksi, Eriophyes spp., for example Eriophyes pyri, Glycyphagus domesticus, Halotydeus destructor, Hemitarsonemus spp., for example Hemitarsonemus latus (=Polyphagotarsonemus latus), Hyalomma spp., Ixodes spp., Latrodectus spp., Loxosceles spp., Neutrombicula autumnalis, Nuphersa spp., Oligonychus spp., for example Oligonychus coniferarum, Oligonychus ilicis, Oligonychus indicus, Oligonychus mangiferus, Oligonychus pratensis, Oligonychus punicae, Oligonychus yothersi, Ornithodorus spp., Ornithonyssus spp., Panonychus spp., for example Panonychus citri (=Metatetranychus citri), Panonychus ulmi (=Metatetranychus ulmi), Phyllocoptruta oleivora, Platytetranychus multidigituli, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., for example Tarsonemus confusus, Tarsonemus pallidus, Tetranychus spp., for example Tetranychus canadensis, Tetranychus cinnabarinus, Tetranychus turkestani, Tetranychus urticae, Trombicula alfreddugesi, Vaejovis spp., Vasates lycopersici;

from the class of the Chilopoda, for example Geophilus spp., Scutigera spp.;

from the order or the class of the Collembola, for example Onychiurus armatus; Sminthurus viridis;

from the class of the Diplopoda, for example Blaniulus guttulatus;

from the class of the Insecta, for example from the order of the Blattodea, for example Blatta orientalis, Blattella asahinai, Blattella germanica, Leucophaea maderae, Panchlora spp., Parcoblatta spp., Periplaneta spp., for example Periplaneta americana, Periplaneta australasiae, Supella longipalpa;

from the order of the Coleoptera, for example Acalymma vittatum, Acanthoscelides obtectus, Adoretus spp., Agelastica alni, Agriotes spp., for example Agriotes linneatus, Agriotes mancus, Alphitobius diaperinus, Amphimallon solstitialis, Anobium punctatum, Anoplophora spp., Anthonomus spp., for example Anthonomus grandis, Anthrenus spp., Apion spp., Apogonia spp., Atomaria spp., for example Atomaria linearis, Attagenus spp., Bans caerulescens, Bruchidius obtectus, Bruchus spp., for example Bruchus pisorum, Bruchus rufimanus, Cassida spp., Cerotoma trifurcata, Ceutorrhynchus spp., for example Ceutorrhynchus assimilis, Ceutorrhynchus quadridens, Ceutorrhynchus rapae, Chaetocnema spp., for example Chaetocnema confinis, Chaetocnema denticulata, Chaetocnema ectypa, Cleonus mendicus, Conoderus spp., Cosmopolites spp., for example Cosmopolites sordidus, Costelytra zealandica, Ctenicera spp., Curculio spp., for example Curculio caryae, Curculio caryatrypes, Curculio obtusus, Curculio sayi, Cryptolestes ferrugineus, Cryptolestes pusillus, Cryptorhynchus lapathi, Cryptorhynchus mangiferae, Cylindrocopturus spp., Cylindrocopturus adspersus, Cylindrocopturus furnissi, Dermestes spp., Diabrotica spp., for example Diabrotica balteata, Diabrotica barberi, Diabrotica undecimpunctata howardi, Diabrotica undecimpunctata undecimpunctata, Diabrotica virgifera virgifera, Diabrotica virgifera zeae, Dichocrocis spp., Dicladispa armigera, Diloboderus spp., Epilachna spp., for example Epilachna borealis, Epilachna varivestis, Epitrix spp., for example Epitrix cucumeris, Epitrix fuscula, Epitrix hirtipennis, Epitrix subcrinita, Epitrix tuberis, Faustinus spp., Gibbium psylloides, Gnathocerus cornutus, Hellula undalis, Heteronychus arator, Heteronyx spp., Hylamorpha elegans, Hylotrupes bajulus, Hypera postica, Hypomeces squamosus, Hypothenemus spp., for example Hypothenemus hampei, Hypothenemus obscurus, Hypothenemus pub escens, Lachnosterna consanguinea, Lasioderma serricorne, Latheticus oryzae, Lathridius spp., Lema spp., Leptinotarsa decemlineata, Leucoptera spp., for example Leucoptera coffeella, Lissorhoptrus oryzophilus, Lixus spp., Luperomorpha xanthodera, Luperodes spp., Lyctus spp., Megascelis spp., Melanotus spp., for example Melanotus longulus oregonensis, Meligethes aeneus, Melolontha spp., for example Melolontha melolontha, Migdolus spp., Monochamus spp., Naupactus xanthographus, Necrobia spp., Niptus hololeucus, Oryctes rhinoceros, Oryzaephilus surinamensis, Oryzaphagus oryzae, Otiorhynchus spp., for example Otiorhynchus cribricollis, Otiorhynchus ligustici, Otiorhynchus ovatus, Otiorhynchus rugosostriarus, Otiorhynchus sulcatus, Oxycetonia jucunda, Phaedon cochleariae, Phyllophaga spp., Phyllophaga helleri, Phyllotreta spp., for example Phyllotreta armoraciae, Phyllotreta pusilla, Phyllotreta ramosa, Phyllotreta striolata, Popillia japonica, Premnotrypes spp., Prostephanus truncatus, Psylliodes spp., for example Psylliodes affinis, Psylliodes chrysocephala, Psylliodes punctulata, Ptinus spp., Rhizobius ventralis, Rhizopertha dominica, Sitophilus spp., for example Sitophilus granarius, Sitophilus linearis, Sitophilus oryzae, Sitophilus zeamais, Sphenophorus spp., Stegobium paniceum, Sternechus spp., for example Sternechus paludatus, Symphyletes spp., Tanymecus spp., for example Tanymecus dilaticollis, Tanymecus indicus, Tanymecus palliatus, Tenebrio molitor, Tenebrioides mauretanicus, Tribolium spp., for example Tribolium audax, Tribolium castaneum, Tribolium confusum, Trogoderma spp., Tychius spp., Xylotrechus spp., Zabrus spp., for example Zabrus tenebrioides; from the order of the Diptera, for example Aedes spp., for example Aedes aegypti, Aedes albopictus, Aedes sticticus, Aedes vexans, Agromyza spp., for example Agromyza frontella, Agromyza parvicornis, Anastrepha spp., Anopheles spp., for example Anopheles quadrimaculatus, Anopheles gambiae, Asphondylia spp., Bactrocera spp., for example Bactrocera cucurbitae, Bactrocera dorsalis, Bactrocera oleae, Bibio hortulanus, Calliphora erythrocephala, Calliphora vicina, Ceratitis capitata, Chironomus spp., Chrysomya spp., Chrysops spp., Chrysozona pluvialis, Cochliomya spp., Contarinia spp., for example Contarinia johnsoni, Contarinia nasturtii, Contarinia pyrivora, Contarinia schulzi, Contarinia sorghicola, Contarinia tritici, Cordylobia anthropophaga, Cricotopus sylvestris, Culex spp., for example Culex pipiens, Culex quinquefasciatus, Culicoides spp., Culiseta spp., Cuterebra spp., Dacus oleae, Dasineura spp., for example Dasineura brassicae, Delia spp., for example Delia antiqua, Delia coarctata, Delia florilega, Delia platura, Delia radicum, Dermatobia hominis, Drosophila spp., for example Drosphila melanogaster, Drosophila suzukii, Echinocnemus spp., Fannia spp., Gasterophilus spp., Glossina spp., Haematopota spp., Hydrellia spp., Hydrellia griseola, Hylemya spp., Hippobosca spp., Hypoderma spp., Liriomyza spp., for example Liriomyza brassicae, Liriomyza huidobrensis, Liriomyza sativae, Lucilia spp., for example Lucilia cuprina, Lutzomyia spp., Mansonia spp., Musca spp., for example Musca domestica, Musca domestica vicina, Oestrus spp., Oscinella frit, Paratanytarsus spp., Paralauterborniella subcincta, Pegomya spp., for example Pegomya betae, Pegomya hyoscyami, Pegomya rubivora, Phlebotomus spp., Phorbia spp., Phormia spp., Piophila casei, Prodiplosis spp., Psila rosae, Rhagoletis spp., for example Rhagoletis cingulata, Rhagoletis completa, Rhagoletis fausta, Rhagoletis indifferens, Rhagoletis mendax, Rhagoletis pomonella, Sarcophaga spp., Simulium spp., for example Simulium meridionale, Stomoxys spp., Tabanus spp., Tetanops spp., Tipula spp., for example Tipula paludosa, Tipula simplex;

from the order of the Hemiptera, for example Acizzia acaciaebaileyanae, Acizzia dodonaeae, Acizzia uncatoides, Acrida turrita, Acyrthosipon spp., for example Acyrthosiphon pisum, Acrogonia spp., Aeneolamia spp., Agonoscena spp., Aleyrodes proletella, Aleurolobus barodensis, Aleurothrixus floccosus, Allocaridara malayensis, Amrasca spp., for example Amrasca bigutulla, Amrasca devastans, Anuraphis cardui, Aonidiella spp., for example Aonidiella aurantii, Aonidiella citrina, Aonidiella inornata, Aphanostigma pin, Aphis spp., for example Aphis citricola, Aphis craccivora, Aphis fabae, Aphis forbesi, Aphis glycines, Aphis gossypii, Aphis hederae, Aphis illinoisensis, Aphis middletoni, Aphis nasturtii, Aphis nerii, Aphis pomi, Aphis spiraecola, Aphis viburniphila, Arboridia apicalis, Arytainilla spp., Aspidiella spp., Aspidiotus spp., for example Aspidiotus nerii, Atanus spp., Aulacorthum solani, Bemisia tabaci, Blastopsylla occidentalis, Boreioglycaspis melaleucae, Brachycaudus helichrysi, Brachycolus spp., Brevicoryne brassicae, Cacopsylla spp., for example Cacopsylla pyricola, Calligypona marginata, Carneocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus ficus, Cicadulina mbila, Coccomytilus halli, Coccus spp., for example Coccus hesperidum, Coccus longulus, Coccus pseudomagnoliarum, Coccus viridis, Cryptomyzus ribis, Cryptoneossa spp., Ctenarytaina spp., Dalbulus spp., Dialeurodes citri, Diaphorina citri, Diaspis spp., Drosicha spp., Dysaphis spp., for example Dysaphis apiifolia, Dysaphis plantaginea, Dysaphis tulipae, Dysmicoccus spp., Empoasca spp., for example Empoasca abrupta, Empoasca fabae, Empoasca maligna, Empoasca solana, Empoasca stevensi, Eriosoma spp., for example Eriosoma americanum, Eriosoma lanigerum, Eriosoma pyricola, Erythroneura spp., Eucalyptolyma spp., Euphyllura spp., Euscelis bilobatus, Ferrisia spp., Geococcus coffeae, Glycaspis spp., Heteropsylla cubana, Heteropsylla spinulosa, Homalodisca coagulata, Hyalopterus arundinis, Hyalopterus pruni, Icerya spp., for example Icerya purchasi, Idiocerus spp., Idioscopus spp., Laodelphax striatellus, Lecanium spp., for example Lecanium corni (=Parthenolecanium corni), Lepidosaphes spp., for example Lepidosaphes ulmi, Lipaphis erysimi, Lycorma delicatula, Macrosiphum spp., for example Macrosiphum euphorbiae, Macrosiphum lilii, Macrosiphum rosae, Macrosteles facifrons, Mahanarva spp., Melanaphis sacchari, Metcalfiella spp., Metcalfa pruinosa, Metopolophium dirhodum, Monellia costalis, Monelliopsis pecanis, Myzus spp., for example Myzus ascalonicus, Myzus cerasi, Myzus ligustri, Myzus ornatus, Myzus persicae, Myzus nicotianae, Nasonovia ribisnigri, Nephotettix spp., for example Nephotettix cincticeps, Nephotettix nigropictus, Nilaparvata lugens, Oncometopia spp., Orthezia praelonga, Oxya chinensis, Pachypsylla spp., Parabemisia myricae, Paratrioza spp., for example Paratrioza cockerelli, Parlatoria spp., Pemphigus spp., for example Pemphigus bursarius, Pemphigus populivenae, Peregrinus maidis, Phenacoccus spp., for example Phenacoccus madeirensis, Phloeomyzus passerinii, Phorodon humuli, Phylloxera spp., for example Phylloxera devastatrix, Phylloxera notabilis, Pinnaspis aspidistrae, Planococcus spp., for example Planococcus citri, Prosopidopsylla flava, Protopulvinaria pyriformis, Pseudaulacaspis pentagona, Pseudococcus spp., for example Pseudococcus calceolariae, Pseudococcus comstocki, Pseudococcus longispinus, Pseudococcus maritimus, Pseudococcus viburni, Psyllopsis spp., Psylla spp., for example Psylla buxi, Psylla mali, Psylla pyri, Pteromalus spp., Pyrilla spp., Quadraspidiotus spp., for example Quadraspidiotus juglansregiae, Quadraspidiotus ostreaeformis, Quadraspidiotus perniciosus, Quesada gigas, Rastrococcus spp., Rhopalosiphum spp., for example Rhopalosiphum maidis, Rhopalosiphum oxyacanthae, Rhopalosiphum padi, Rhopalosiphum rufiabdominale, Saissetia spp., for example Saissetia coffeae, Saissetia miranda, Saissetia neglecta, Saissetia oleae, Scaphoideus titanus, Schizaphis graminum, Selenaspidus articulatus, Sitobion avenae, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., for example Toxoptera aurantii, Toxoptera citricidus, Trialeurodes vaporariorum, Trioza spp., for example Trioza diospyri, Typhlocyba spp., Unaspis spp., Viteus vitifolii, Zygina spp.;

from the suborder of the Heteroptera, for example Anasa tristis, Antestiopsis spp., Boisea spp., Blissus spp., Calocoris spp., Campylomma livida, Cavelerius spp., Cimex spp., for example Cimex adjunctus, Cimex hemipterus, Cimex lectularius, Cimex pilosellus, Collaria spp., Creontiades dilutus, Dasynus piperis, Dichelops furcatus, Diconocoris hewetti, Dysdercus spp., Euschistus spp., for example Euschistus heros, Euschistus servus, Euschistus tristigmus, Euschistus variolarius, Eurygaster spp., Halyomorpha halys, Heliopeltis spp., Horcias nobilellus, Leptocorisa spp., Leptocorisa varicornis, Leptoglossus occidentalis, Leptoglossus phyllopus, Lygocoris spp., for example Lygocoris pabulinus, Lygus spp., for example Lygus elisus, Lygus hesperus, Lygus lineolaris, Macropes excavatus, Monalonion atratum, Nezara spp., for example Nezara viridula, Oebalus spp., Piesma quadrata, Piezodorus spp., for example Piezodorus guildinii, Psallus spp., Pseudacysta persea, Rhodnius spp., Sahlbergella singularis, Scaptocoris castanea, Scotinophora spp., Stephanitis nashi, Tibraca spp., Triatoma spp.;

from the order of the Hymenoptera, for example Acromyrmex spp., Athalia spp., for example Athalia rosae, Atta spp., Diprion spp., for example Diprion similis, Hoplocampa spp., for example Hoplocampa cookei, Hoplocampa testudinea, Lasius spp., Monomorium pharaonis, Sirex spp., Solenopsis invicta, Tapinoma spp., Urocerus spp., Vespa spp., for example Vespa crabro, Xeris spp.;

from the order of the Isopoda, for example Armadillidium vulgare, Oniscus asellus, Porcellio scaber;

from the order of the Isoptera, for example Coptotermes spp., for example Coptotermes formosanus, Cornitermes cumulans, Cryptotermes spp., Incisitermes spp., Microtermes obesi, Odontotermes spp., Reticulitermes spp., for example Reticulitermes flavipes, Reticulitermes hesperus; from the order of the Lepidoptera, for example Achroia grisella, Acronicta major, Adoxophyes spp., for example Adoxophyes orana, Aedia leucomelas, Agrotis spp., for example Agrotis segetum, Agrotis ipsilon, Alabama spp., for example Alabama argillacea, Amyelois transitella, Anarsia spp., Anticarsia spp., for example Anticarsia gemmatalis, Argyroploce spp., Barathra brassicae, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., for example Chilo plejadellus, Chilo suppressalis, Choristoneura spp., Clysia ambiguella, Cnaphalocerus spp., Cnaphalocrocis medinalis, Cnephasia spp., Conopomorpha spp., Conotrachelus spp., Copitarsia spp., Cydia spp., for example Cydia nigricana, Cydia pomonella, Dalaca noctuides, Diaphania spp., Diatraea saccharalis, Earias spp., Ecdytolopha aurantium, Elasmopalpus lignosellus, Eldana saccharina, Ephestia spp., for example Ephestia elutella, Ephestia kuehniella, Epinotia spp., Epiphyas postvittana, Etiella spp., Eulia spp., Eupoecilia ambiguella, Euproctis spp., for example Euproctis chrysorrhoea, Euxoa spp., Feltia spp., Galleria mellonella, Gracillaria spp., Grapholitha spp., for example Grapholita molesta, Grapholita prunivora, Hedylepta spp., Helicoverpa spp., for example Helicoverpa armigera, Helicoverpa zea, Heliothis spp., for example Heliothis virescens Hofmannophila pseudospretella, Homoeosoma spp., Homona spp., Hyponomeuta padella, Kakivoria flavofasciata, Laphygma spp., Leucinodes orbonalis, Leucoptera spp., for example Leucoptera coffeella, Lithocolletis spp., for example Lithocolletis blancardella, Lithophane antennata, Lobesia spp., for example Lobesia botrana, Loxagrotis albicosta, Lymantria spp., for example Lymantria dispar, Lyonetia spp., for example Lyonetia clerkella, Malacosoma neustria, Maruca testulalis, Mamestra brassicae, Melanitis leda, Mocis spp., Monopis obviella, Mythimna separata, Nemapogon cloacellus, Nymphula spp., Oiketicus spp., Oria spp., Orthaga spp., Ostrinia spp., for example Ostrinia nubilalis, Oulema melanopus, Oulema oryzae, Panolis flammea, Parnara spp., Pectinophora spp., for example Pectinophora gossypiella, Perileucoptera spp., Phthorimaea spp., for example Phthorimaea operculella, Phyllocnistis citrella, Phyllonorycter spp., for example Phyllonorycter blancardella, Phyllonorycter crataegella, Pieris spp., for example Pieris rapae, Platynota stultana, Plodia interpunctella, Plusia spp., Plutella xylostella (=Plutella maculipennis), Prays spp., Prodenia spp., Protoparce spp., Pseudaletia spp., for example Pseudaletia unipuncta, Pseudoplusia includens, Pyrausta nubilalis, Rachiplusia nu, Schoenobius spp., for example Schoenobius bipunctifer, Scirpophaga spp., for example Scirpophaga innotata, Scotia segetum, Sesamia spp., for example Sesamia inferens, Sparganothis spp., Spodoptera spp., for example Spodoptera eradiana, Spodoptera exigua, Spodoptera frugiperda, Spodoptera praefica, Stathmopoda spp., Stomopteryx subsecivella, Synanthedon spp., Tecia solanivora, Thermesia gemmatalis, Tinea cloacella, Tinea pellionella, Tineola bisselliella, Tortrix spp., Trichophaga tapetzella, Trichoplusia spp., for example Trichoplusia ni, Tryporyza incertulas, Tuta absoluta, Virachola spp.;

from the order of the Orthoptera or Saltatoria, for example Acheta domesticus, Dichroplus spp., Gryllotalpa spp., for example Gryllotalpa gryllotalpa, Hieroglyphus spp., Locusta spp., for example Locusta migratoria, Melanoplus spp., for example Melanoplus devastator, Paratlanticus ussuriensis, Schistocerca gregaria;

from the order of the Phthiraptera, for example Damalinia spp., Haematopinus spp., Linognathus spp., Pediculus spp., Phylloxera vastatrix, Phthirus pubis, Trichodectes spp.;

from the order of the Psocoptera, for example Lepinotus spp., Liposcelis spp.;

from the order of the Siphonaptera, for example, Ceratophyllus spp., Ctenocephalides spp., for example Ctenocephalides canis, Ctenocephalides felis, Pulex irritans, Tunga penetrans, Xenopsylla cheopis;

from the order of the Thysanoptera, for example Anaphothrips obscurus, Baliothrips biformis, Drepanothrips reuteri, Enneothrips flavens, Frankliniella spp., for example Frankliniella fusca, Frankliniella occidentalis, Frankliniella schultzei, Frankliniella tritici, Frankliniella vaccinii, Frankliniella williamsi, Heliothrips spp., Hercinothrips femoralis, Rhipiphorothrips cruentatus, Scirtothrips spp., Taeniothrips cardamomi, Thrips spp., for example Thrips palmi, Thrips tabaci;

from the order of the Zygentoma (=Thysanura), for example Ctenolepisma spp., Lepisma saccharina, Lepismodes inquilinus, Thermobia domestica;

from the class of the Symphyla, for example Scutigerella spp., for example Scutigerella immaculata;

pests from the phylum of the Mollusca, for example from the class of the Bivalvia, for example Dreissena spp.,

and also from the class of the Gastropoda, for example Anon spp., for example Anon ater rufus, Biomphalaria spp., Bulinus spp., Deroceras spp., for example Deroceras laeve, Galba spp., Lymnaea spp., Oncomelania spp., Pomacea spp., Succinea spp.;

animal and human parasites from the phyla of the Platyhelminthes and Nematoda, for example Aelurostrongylus spp., Amidostomum spp., Ancylostoma spp, Angiostrongylus spp., Anisakis spp., Anoplocephala spp., Ascaris spp., Ascaridia spp., Baylisascaris spp., Brugia spp., Bunostomum spp., Capillaria spp., Chabertia spp., Clonorchis spp., Cooperia spp., Crenosoma spp., Cyathostoma spp., Dicrocoelium spp., Dictyocaulus spp., Diphyllobothrium spp., Dipylidium spp., Dirofilaria spp., Dracunculus spp., Echinococcus spp., Echinostoma spp., Enterobius spp., Eucoleus spp., Fasciola spp., Fascioloides spp., Fasciolopsis spp., Filaroides spp., Gongylonema spp., Gyrodactylus spp., Habronema spp., Haemonchus spp., Heligmosomoides spp., Heterakis spp., Hymenolepis spp., Hyostrongylus spp., Litomosoides spp., Loa spp., Metastrongylus spp., Metorchis spp., Mesocestoides spp., Moniezia spp., Muellerius spp., Necator spp., Nematodirus spp., Nippostrongylus spp., Oesophagostomum spp., Ollulanus spp., Onchocerca spp, Opisthorchis spp., Oslerus spp., Ostertagia spp., Oxyuris spp., Paracapillaria spp., Parafilaria spp., Paragonimus spp., Paramphistomum spp., Paranoplocephala spp., Parascaris spp., Passalurus spp., Protostrongylus spp., Schistosoma spp., Setaria spp., Spirocerca spp., Stephanofilaria spp., Stephanurus spp., Strongyloides spp., Strongylus spp., Syngamus spp., Taenia spp., Teladorsagia spp., Thelazia spp., Toxascaris spp., Toxocara spp., Trichinella spp., Trichobilharzia spp., Trichostrongylus spp., Trichuris spp., Uncinaria spp., Wuchereria spp.;

plant pests from the phylum of the Nematoda, i.e. phytoparasitic nematodes, especially Aglenchus spp., for example Aglenchus agricola, Anguina spp., for example Anguina tritici, Aphelenchoides spp., for example Aphelenchoides arachidis, Aphelenchoides fragariae, Belonolaimus spp., for example Belonolaimus gracilis, Belonolaimus longicaudatus, Belonolaimus nortoni, Bursaphelenchus spp., for example Bursaphelenchus cocophilus, Bursaphelenchus eremus, Bursaphelenchus xylophilus, Cacopaurus spp., for example Cacopaurus pestis, Criconemella spp., for example Criconemella curvata, Criconemella onoensis, Criconemella ornata, Criconemella rusium, Criconemella xenoplax Mesocriconema xenoplax), Criconemoides spp., for example Criconemoides ferniae, Criconemoides onoense, Criconemoides ornatum, Ditylenchus spp., for example Ditylenchus dipsaci, Dolichodorus spp., Globodera spp., for example Globodera pallida, Globodera rostochiensis, Helicotylenchus spp., for example Helicotylenchus dihystera, Hemicriconemoides spp., Hemicycliophora spp., Heterodera spp., for example Heterodera avenae, Heterodera glycines, Heterodera schachtii, Hoplolaimus spp., Longidorus spp., for example Longidorus africanus, Meloidogyne spp., for example Meloidogyne chitwoodi, Meloidogyne fallax, Meloidogyne hapla, Meloidogyne incognita, Meloinema spp., Nacobbus spp., Neotylenchus spp., Paraphelenchus spp., Paratrichodorus spp., for example Paratrichodorus minor, Pratylenchus spp., for example Pratylenchus penetrans, Pseudohalenchus spp., Psilenchus spp., Punctodera spp., Quinisulcius spp., Radopholus spp., for example Radopholus citrophilus, Radopholus similis, Rotylenchulus spp., Rotylenchus spp., Scutellonema spp., Subanguina spp., Trichodorus spp., for example Trichodorus obtusus, Trichodorus primitivus, Tylenchorhynchus spp., for example Tylenchorhynchus annulatus, Tylenchulus spp., for example Tylenchulus semipenetrans, Xiphinema spp., for example Xiphinema index.

In addition, it is possible to control, from the sub-kingdom of the Protozoa, the order of the Coccidia, for example Eimeria spp.

The compounds of the formula (I) can optionally, at certain concentrations or application rates, also be used as herbicides, safeners, growth regulators or agents to improve plant properties, as microbicides or gametocides, for example as fungicides, antimycotics, bactericides, virucides (including agents against viroids) or as agents against MLO (mycoplasma-like organisms) and RLO (rickettsia-like organisms). If appropriate, they can also be used as intermediates or precursors for the synthesis of other active ingredients.

The present invention further relates to formulations and use forms prepared therefrom as crop protection compositions and/or pesticides, for example drench, drip and spray liquors, comprising at least one of the inventive active ingredients. In some cases, the use forms comprise further crop protection compositions and/or pesticides and/or adjuvants which improve action, such as penetrants, e.g. vegetable oils, for example rapeseed oil, sunflower oil, mineral oils, for example paraffin oils, alkyl esters of vegetable fatty acids, for example rapeseed oil methyl ester or soya oil methyl ester, or alkanol alkoxylates and/or spreaders, for example alkylsiloxanes and/or salts, for example organic or inorganic ammonium or phosphonium salts, for example ammonium sulphate or diammonium hydrogenphosphate and/or retention promoters, for example dioctyl sulphosuccinate or hydroxypropyl guar polymers and/or humectants, for example glycerol and/or fertilizers, for example ammonium-, potassium- or phosphorus-containing fertilizers.

Customary formulations are, for example, water-soluble liquids (SL), emulsion concentrates (EC), emulsions in water (EW), suspension concentrates (SC, SE, FS, OD), water-dispersible granules (WG), granules (GR) and capsule concentrates (CS); these and further possible formulation types are described, for example, by Crop Life International and in Pesticide Specifications, Manual on development and use of FAO and WHO specifications for pesticides, FAO Plant Production and Protection Papers—173, prepared by the FAO/WHO Joint Meeting on Pesticide Specifications, 2004, ISBN: 9251048576. The formulations optionally comprise, as well as one or more inventive active ingredients, further active agrochemical ingredients.

These are preferably formulations or use forms which comprise auxiliaries, for example extenders, solvents, spontaneity promoters, carriers, emulsifiers, dispersants, antifreezes, biocides, thickeners and/or further auxiliaries, for example adjuvants. An adjuvant in this context is a component which enhances the biological effect of the formulation, without the component itself having any biological effect. Examples of adjuvants are agents which promote retention, spreading, attachment to the leaf surface or penetration.

These formulations are prepared in a known manner, for example by mixing the active ingredients with auxiliaries, for example extenders, solvents and/or solid carriers, and/or further auxiliaries, for example surfactants. The formulations are produced either in suitable production plants or else before or during application.

Auxiliaries used may be substances capable of imparting particular properties, such as certain physical, technical and/or biological properties, to the formulation of the active ingredient, or the application forms prepared from these formulations (such as ready-to-use crop protection compositions, for example, such as spray liquors or seed dressings).

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, or else water.

In principle, it is possible to use all suitable solvents. Examples of suitable solvents are aromatic hydrocarbons, for example xylene, toluene or alkylnaphthalenes, chlorinated aromatic or chlorinated aliphatic hydrocarbons, for example chlorobenzene, chloroethylene or methylene chloride, aliphatic hydrocarbons, for example cyclohexane, paraffins, petroleum fractions, mineral and vegetable oils, alcohols, for example methanol, ethanol, isopropanol, butanol or glycol and the ethers and esters thereof, ketones, for example acetone, methyl ethyl ketone, methyl isobutyl ketone or cyclohexanone, strongly polar solvents such as dimethyl sulphoxide, and water.

In principle, it is possible to use all suitable carriers. Useful carriers especially include: for example ammonium salts and ground natural minerals such as kaolins, clays, talc, chalk, quartz, attapulgite, montmorillonite or diatomaceous earth, and ground synthetic materials such as finely divided silica, alumina and natural or synthetic silicates, resins, waxes and/or solid fertilizers. Mixtures of such carriers may also be used. Useful carriers for granules include: for example crushed and fractionated natural rocks such as calcite, marble, pumice, sepiolite, dolomite, and synthetic granules of inorganic and organic meals, and also granules of organic material such as sawdust, paper, coconut shells, corn cobs and tobacco stalks.

Liquefied gaseous extenders or solvents can also be used. Particularly suitable extenders or carriers are those which are gaseous at ambient temperature and under atmospheric pressure, for example aerosol propellant gases, such as halohydrocarbons, and also butane, propane, nitrogen and carbon dioxide.

Examples of emulsifiers and/or foam generators, dispersants or wetting agents having ionic or nonionic properties, or mixtures of these surfactants, are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyl taurates), phosphoric esters of polyethoxylated alcohols or phenols, fatty acid esters of polyols, and derivatives of the compounds comprising sulphates, sulphonates and phosphates, e.g. alkylaryl polyglycol ethers, alkylsulphonates, alkyl sulphates, arylsulphonates, protein hydrolysates, lignosulphite waste liquors, and methyl cellulose. The presence of a surfactant is advantageous when one of the active ingredients and/or one of the inert carriers is insoluble in water and when application is effected in water.

Further auxiliaries which may be present in the formulations and the use forms derived therefrom include dyes such as inorganic pigments, for example iron oxide, titanium oxide and Prussian Blue, and organic dyes such as alizarin dyes, azo dyes and metal phthalocyanine dyes, and nutrients and trace nutrients such as salts of iron, manganese, boron, copper, cobalt, molybdenum and zinc.

Additional components may be stabilizers, such as cold stabilizers, preservatives, antioxidants, light stabilizers, or other agents which improve chemical and/or physical stability. Foam formers or antifoams may also be present.

In addition, the formulations and the use forms derived therefrom may also comprise, as additional auxiliaries, stickers such as carboxymethyl cellulose and natural and synthetic polymers in the form of powders, granules or latices, such as gum arabic, polyvinyl alcohol and polyvinyl acetate, or else natural phospholipids such as cephalins and lecithins and synthetic phospholipids. Further auxiliaries may be mineral and vegetable oils.

Optionally, further auxiliaries may be present in the formulations and the use forms derived therefrom. Examples of such additives include fragrances, protective colloids, binders, adhesives, thickeners, thixotropic agents, penetrants, retention promoters, stabilizers, sequestrants, complexing agents, humectants, spreaders. In general, the active ingredients can be combined with any solid or liquid additive which is commonly used for formulation purposes.

Useful retention promoters include all those substances which reduce the dynamic surface tension, for example dioctyl sulphosuccinate, or increase the viscoelasticity, for example hydroxypropylguar polymers.

Useful penetrants in the present context are all those substances which are typically used to improve the penetration of active agrochemical ingredients into plants. Penetrants are defined in this context by their ability to penetrate from the (generally aqueous) application liquor and/or from the spray coating into the cuticle of the plant and thereby increase the mobility of active ingredients in the cuticle. The method described in the literature (Baur et al., Pesticide Science 1997, 51, 131) can be used to determine this property. Examples include alcohol alkoxylates such as coconut fatty ethoxylate (10) or isotridecyl ethoxylate (12), fatty acid esters, for example rapeseed oil methyl ester or soya oil methyl ester, fatty amine alkoxylates, for example tallowamine ethoxylate (15), or ammonium and/or phosphonium salts, for example ammonium sulphate or diammonium hydrogenphosphate.

In a preferred embodiment of the invention, a penetrant is additionally added to the crop protection compositions to enhance the action. Suitable penetrants also include, for example, substances which promote the availability of the compounds of the formula (I) in the spray coating. These include, for example, mineral and vegetable oils. Useful oils include all mineral or vegetable oils—modified or otherwise—which are typically usable in agrochemical compositions. Examples include sunflower oil, rapeseed oil, olive oil, castor oil, colza oil, cornseed oil, cottonseed oil and soybean oil or the esters of the oils mentioned. Preference is given to rapeseed oil, sunflower oil and their methyl or ethyl esters, especially rapeseed oil methyl ester.

The concentration of penetrant in the inventive compositions can be varied within a wide range. In the case of a formulated crop protection composition, it is generally 1 to 95% by weight, preferably 1 to 55% by weight, more preferably 15-40% by weight. In the ready-to-use compositions (spray liquors), the concentrations are generally between 0.1 and 10 g/l, preferably between 0.5 and 5 g/l.

The formulations contain preferably between 0.00000001% and 98% by weight of active ingredient or more preferably between 0.01% and 95% by weight of active ingredient, more preferably between 0.5% and 90% by weight of active ingredient, based on the weight of the formulation.

The active ingredient content of the use forms (crop protection compositions) prepared from the formulations can vary within wide limits. The active ingredient concentration of the use forms may typically be between 0.00000001% and 95% by weight of active ingredient, preferably between 0.00001% and 1% by weight, based on the weight of the use form. The compounds are applied in a customary manner appropriate for the use forms.

The inventive active ingredients can be used as such or in formulations thereof, including in a mixture with one or more suitable fungicides, bactericides, acaricides, nematicides, insecticides, microbicides, fertilizers, attractants, sterilants, synergists, safeners, semiochemicals and/or plant growth regulators, in order thus, for example, to broaden the spectrum of action, to prolong the duration of action, to increase the rate of action, to prevent repulsion or prevent evolution of resistance. In addition, combinations of this kind can improve plant growth, increase tolerance to high or low temperatures, to drought or to increased levels of water and/or soil salinity, improve flowering performance, facilitate harvesting and increase yields, accelerate ripening, increase the quality and/or nutritional value of the harvested products, prolong storage life and/or improve the processibility of the harvested products. In general, by combining the inventive active ingredients and mixing partners, synergistic effects are obtained, meaning that the efficacy of the mixture in question is greater than the efficacy of the individual components. In general, the combinations can be used either as seed treatments or else in premixes, tankmixes or readymixes.

The active ingredients identified here by their common name are known and are described, for example, in the pesticide handbook (“The Pesticide Manual” 14th Ed., British Crop Protection Council 2006) or can be found on the Internet (e.g. http://www.alanwood.net/pesticides).

Insecticides/acaricides/nematicides suitable as mixing components are:

(1) Acetylcholinesterase (AChE) inhibitors, for example

carbamates, e.g. alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or

organophosphates, e.g. acephate, azamethiphos, azinphos-ethyl, azinphos-methyl, cadusafos, chlorethoxyfos, chlorfenvinphos, chlormephos, chloropyrifos, chloropyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl)salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon and vamidothion.

(2) GABA-gated chloride channel antagonists, for example

cyclodiene organochlorines, e.g. chlordane and endosulfan; or

phenylpyrazoles (fiproles), e.g. ethiprole and fipronil.

(3) Sodium channel modulators/voltage-dependent sodium channel blockers, for example

pyrethroids, e.g. acrinathrin, allethrin, d-cis-trans allethrin, d-trans allethrin, bifenthrin, bioallethrin, bioallethrin S-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans isomers], deltamethrin, empenthrin [(EZ)-(1R) isomers), esfenvalerate, etofenprox, fenpropathrin, fenvalerate, flucythrinate, flumethrin, tau-fluvalinate, halfenprox, imiprothrin, kadethrin, permethrin, phenothrin [(1R)-trans isomer), prallethrin, pyrethrine (pyrethrum), resmethrin, silafluofen, tefluthrin, tetramethrin, tetramethrin [(1R) isomers)], tralomethrin and transfluthrin; or

DDT; or methoxychlor.

(4) Nicotinergic acetylcholine receptor (nAChR) agonists, for example

neonicotinoids, for example acetamiprid, clothianidin, dinotefuran, imidacloprid, nitenpyram, thiacloprid and thiamethoxam; or

nicotine; or

sulfoxaflor.

(5) Nicotinergic acetylcholine receptor (nAChR) allosteric activators, for example spinosyns, for example spinetoram and spinosad.

(6) Chloride channel activators, for example

avermectins/milbemycins, for example abamectin, emamectin benzoate, lepimectin and milbemectin.

(7) Juvenile hormone imitators, for example

juvenile hormone analogues, e.g. hydroprene, kinoprene and methoprene; or

fenoxycarb; or pyriproxyfen.

(8) Active ingredients with unknown or nonspecific mechanisms of action, for example

alkyl halides, e.g. methyl bromide and other alkyl halides; or

chloropicrin; or sulphuryl fluoride; or borax; or tartar emetic.

(9) Selective antifeedants, for example pymetrozine; or flonicamid.

(10) Mite growth inhibitors, for example clofentezine, hexythiazox and diflovidazin; or etoxazole.

(11) Microbial disruptors of the insect gut membrane, e.g. Bacillus thuringiensis subspecies israelensis, Bacillus thuringiensis subspecies aizawai, Bacillus thuringiensis subspecies kurstaki, Bacillus thuringiensis subspecies tenebrionis and B.t. plant proteins: Cry1Ab, Cry1Ac, Cry1Fa, Cry1A.105, Cry2Ab, Vip3A, mCry3A, Cry3Ab, Cry3Bb, Cry34 Ab1/35Ab1; or

Bacillus sphaericus.

(12) Oxidative phosphorylation inhibitors, ATP disruptors, for example diafenthiuron; or

organotin compounds, e.g. azocyclotin, cyhexatin and fenbutatin oxide; or

propargite; or tetradifon.

(13) Oxidative phosphorylation decouplers that disrupt the H proton gradient, for example, chlorfenapyr, DNOC and sulfluramid.

(14) Nicotinergic acetylcholine receptor antagonists, for example bensultap, cartap hydrochloride, thiocyclam, and thiosultap-sodium.

(15) Chitin biosynthesis inhibitors, type 0, for example bistrifluron, chlorfluazuron, diflubenzuron, flucycloxuron, flufenoxuron, hexaflumuron, lufenuron, novaluron, noviflumuron, teflubenzuron and triflumuron.

(16) Chitin biosynthesis inhibitors, type 1, for example buprofezin.

(17) Molting disruptors, dipteran, for example cyromazine.

(18) Ecdysone receptor agonists, for example chromafenozide, halofenozide, methoxyfenozide and tebufenozide.

(19) Octopaminergic agonists, for example amitraz.

(20) Complex-III electron transport inhibitors, for example hydramethylnone; or acequinocyl; or fluacrypyrim.

(21) Complex-I electron transport inhibitors, for example

METI acaricides, e.g. fenazaquin, fenpyroximate, pyrimidifen, pyridaben, tebufenpyrad and tolfenpyrad; or

rotenone (Derris).

(22) Voltage-dependent sodium channel blockers, for example indoxacarb; or metaflumizone.

(23) Inhibitors of acetyl-CoA carboxylase, for example

tetronic and tetramic acid derivatives, for example spirodiclofen, spiromesifen and spirotetramat.

(24) Complex-IV electron transport inhibitors, for example

phosphines, for example aluminium phosphide, calcium phosphide, phosphine and zinc phosphide; or cyanide.

(25) Complex-II electron transport inhibitors, for example cyenopyrafen and cyflumetofen.

(28) Ryanodine receptor effectors, for example

diamides, e.g. chlorantraniliprole, cyantraniliprole and flubendiamide.

Further active ingredients having an unknown mechanism of action, for example amidoflumet, azadirachtin, benclothiaz, benzoximate, bifenazate, bromopropylate, chinomethionat, cryolite, dicofol, diflovidazin, fluensulphone, flufenerim, flufiprole, fluopyram, fufenozide, imidaclothiz, iprodione, meperfluthrin, pyridalyl, pyrifluquinazon, tetramethylfluthrin and iodomethane; and additionally preparations based on Bacillus firmus (particularly strain CNCM 1-1582, for example VOTiVO™, BioNem), and the following known active compounds:

3-bromo-N-{2-bromo-4-chlor-6-[(1-cyclopropylethyl)carbamoyl]phenyl}-1-(3-chloropyridin-2-yl)-1H-pyrazole-5-carboxamide (known from WO 2005/077934), 4-{[(6-bromopyrid-3-yl)methyl](2-fluoroethyl)amino}furan-2(5H)-one (known from WO 2007/115644), 4-{[(6-fluoropyrid-3-yl)methyl](2,2-difluoroethyl)amino}furan-2(5H)-one (known from WO 2007/115644), 4-{[(2-chloro-1,3-thiazol-5-yl)methyl](2-fluoroethyl)amino}furan-2(5H)-one (known from WO 2007/115644), 4-{[(6-chloropyrid-3-yl)methyl](2-fluoroethyl)amino}furan-2(5H)-one (known from WO 2007/115644), flupyradifurone, 4-{[(6-chloro-5-fluoropyrid-3-yl)methyl](methyl)amino}furan-2(5H)-one (known from WO 2007/115643), 4-{[(5,6-dichloropyrid-3-yl)methyl](2-fluoroethyl)amino}furan-2(5H)-one (known from WO 2007/115646), 4-{[(6-chloro-5-fluoropyrid-3-yl)methyl](cyclopropyl)amino}furan-2(5H)-one (known from WO 2007/115643), 4-{[(6-chloropyrid-3-yl)methyl](cyclopropyl)amino}furan-2(5H)-one (known from EPA 0 539 588), 4-{[(6-chloropyrid-3-yl)methyl](methyl)amino}furan-2(5H)-one (known from EP A 0 539 588), {[1-(6-chloropyridin-3-yl)ethyl](methyl)oxido-λ4-sulphanylidene}cyanamide (known from WO2007/149134) and its diastereomers {[(1R)-1-(6-chloropyridin-3-yl)ethyl](methyl)oxido-λ4-sulphanylidene}cyanamide (A) and {[(1S)-1-(6-chloropyridin-3-yl)ethyl](methyl)oxido-λ4-sulphanylidene}cyanamide (B) (likewise known from WO 2007/149134) and also diastereomers [(R)-methyl(oxido) {(1R)-1-[6-(trifluoromethyl)pyridin-3-yl]ethyl}-λ4-sulphanylidene]cyanamide (A1) and [(S)-methyl(oxido) {(1S)-1-[6-(trifluoromethyl)pyridin-3-yl]ethyl}-λ4-sulphanylidene]cyanamide (A2), identified as diastereomer group A (known from WO 2010/074747, WO 2010/074751), [(R)-methyl(oxido) {(1S)-1-[6-(trifluoromethyl)pyridin-3-yl]ethyl}-λ4-sulphanylidene]cyanamide (B1) and [(S)-methyl(oxido) {(1R)-1-[6-(trifluoromethyl)pyridin-3-yl]ethyl}-λ4-sulphanylidene]cyanamide (B2), identified as diastereomer group B (likewise known from WO 2010/074747, WO 2010/074751) and 11-(4-chloro-2,6-dimethylphenyl)-12-hydroxy-1,4-dioxa-9-azadispiro[4.2.4.2]tetradec-11-en-10-one (known from WO 2006/089633), 3-(4′-fluoro-2,4-dimethylbiphenyl-3-yl)-4-hydroxy-8-oxa-1-azaspiro[4.5]dec-3-en-2-one (known from WO 2008/067911), 1-{2-fluoro-4-methyl-5-[(2,2,2-trifluoroethyl)sulphinyl]phenyl}-3-(trifluoromethyl)-1H-1,2,4-triazol-5-amine (known from WO 2006/043635), afidopyropen (known from WO 2008/066153), 2-cyano-3-(difluoromethoxy)-N,N-dimethylbenzolsulphonamide (known from WO 2006/056433), 2-cyano-3-(difluoromethoxy)-N-methylbenzolsulphonamide (known from WO 2006/100288), 2-cyano-3-(difluoromethoxy)-N-ethylbenzenesulphonamide (known from WO 2005/035486), 4-(difluoromethoxy)-N-ethyl-N-methyl-1,2-benzothiazol-3-amine-1,1-dioxide (known from WO 2007/057407), N-[1-(2,3-dimethylphenyl)-2-(3,5-dimethylphenyl)ethyl]-4,5-dihydro-1,3-thiazol-2-amine (known from WO 2008/104503), {1′-[(2E)-3-(4-chlorophenyl)prop-2-en-1-yl]-5-fluorospiro[indol-3,4′-piperidine]-1(2H)-yl}(2-chloropyridin-4-yl)methanone (known from WO 2003/106457), 3-(2,5-dimethylphenyl)-4-hydroxy-8-methoxy-1,8-diazaspiro[4.5]dec-3-en-2-one (known from WO 2009/049851), 3-(2,5-dimethylphenyl)-8-methoxy-2-oxo-1,8-diazaspiro[4.5]dec-3-en-4-yl ethyl carbonate (known from WO 2009/049851), 4-(but-2-yn-1-yloxy)-6-(3,5-dimethylpiperidin-1-yl)-5-fluoropyrimidine (known from WO 2004/099160), (2,2,3,3,4,4,5,5-octafluoropentyl)(3,3,3-trifluoropropyl)malononitrile (known from WO 2005/063094), (2,2,3,3,4,4,5,5-octafluoropentyl)(3,3,4,4,4-pentafluorobutyl)malononitrile (known from WO 2005/063094), 8-[2-(cyclopropylmethoxy)-4-(trifluoromethyl)phenoxy]-3-[6-(trifluoromethyl)pyridazin-3-yl]-3-azabicyclo[3.2.1]octane (known from WO 2007/040280), flometoquin, PF1364 (CAS Reg. No. 1204776-60-2) (known from JP 2010/018586), 5-[5-(3,5-dichlorophenyl)-5-(trifluoromethyl)-4,5-dihydro-1,2-oxazol-3-yl]-2-(1H-1,2,4-triazol-1-yl)benzonitrile (known from WO 2007/075459), 5-[5-(2-chloropyridin-4-yl)-5-(trifluoromethyl)-4,5-dihydro-1,2-oxazol-3-yl]-2-(1H-1,2,4-triazol-1-yl)benzonitrile (known from WO 2007/075459), 4-[5-(3,5-dichlorophenyl)-5-(trifluoromethyl)-4,5-dihydro-1,2-oxazol-3-yl]-2-methyl-N-{2-oxo-2-[(2,2,2-trifluoroethyl)amino]ethyl}benzamide (known from WO 2005/085216), 4-{[(6-chloropyridin-3-yl)methyl](cyclopropyl)amino}-1,3-oxazol-2(5H)-one, 4-{[(6-chloropyridin-3-yl)methyl](2,2-difluoroethyl)amino}-1,3-oxazol-2(5H)-one, 4-{[(6-chloropyridin-3-yl)methyl](ethyl)amino}-1,3-oxazol-2(5H)-one, 4-{[(6-chloropyridin-3-yl)methyl](methyl)amino}-1,3-oxazol-2(5H)-one (all known from WO 2010/005692), pyflubumide (known from WO 2002/096882), methyl 2-[2-({[3-bromo-1-(3-chloropyridin-2-yl)-1H-pyrazol-5-yl]carbonyl}amino)-5-chloro-3-methylbenzoyl]-2-methylhydrazinecarboxylate (known from WO 2005/085216), methyl 2-[2-({[3-bromo-1-(3-chloropyridin-2-yl)-1H-pyrazol-5-yl]carbonyl}amino)-5-cyano-3-methylbenzoyl]-2-ethylhydrazinecarboxylate (known from WO 2005/085216), methyl 2-[2-({[3-bromo-1-(3-chloropyridin-2-yl)-1H-pyrazol-5-yl]carbonyl}amino)-5-cyano-3-methylbenzoyl]-2-methylhydrazinecarboxylate (known from WO 2005/085216), methyl 2-[3,5-dibromo-2-({[3-bromo-1-(3-chloropyridin-2-yl)-1H-pyrazol-5-yl]carbonyl}amino)benzoyl]-1,2-diethylhydrazinecarboxylate (known from WO 2005/085216), methyl 2-[3,5-dibromo-2-({[3-bromo-1-(3-chloropyridin-2-yl)-1H-pyrazol-5-yl]carbonyl}amino)benzoyl]-2-ethylhydrazinecarboxylate (known from WO 2005/085216), (5RS,7RS;5RS,7SR)-1-(6-chloro-3-pyridylmethyl)-1,2,3,5,6,7-hexahydro-7-methyl-8-nitro-5-propoxyimidazo[1,2-a]pyridine (known from WO 2007/101369), 2-{6-[2-(5-fluoropyridin-3-yl)-1,3-thiazol-5-yl]pyridin-2-yl}pyrimidine (known from WO 2010/006713), 2-{6-[2-(pyridin-3-yl)-1,3-thiazol-5-yl]pyridin-2-yl}pyrimidine (known from WO 2010/006713), 1-(3-chloropyridin-2-yl)-N-[4-cyano-2-methyl-6-(methylcarbamoyl)phenyl]-3-{[5-(trifluoromethyl)-1H-tetrazol-1-yl]methyl}-1H-pyrazole-5-carboxamide (known from WO 2010/069502), 1-(3-chloropyridin-2-yl)-N-[4-cyano-2-methyl-6-(methylcarbamoyl)phenyl]-3-{[5-(trifluoromethyl)-2H-tetrazol-2-yl]methyl}-1H-pyrazole-5-carboxamide (known from WO 2010/069502), N-[2-(tert-butylcarbamoyl)-4-cyano-6-methylphenyl]-1-(3-chloropyridin-2-yl)-3-{[5-(trifluoromethyl)-1H-tetrazol-1-yl]methyl}-1H-pyrazole-5-carboxamide (known from WO 2010/069502), N-[2-(tert-butylcarbamoyl)-4-cyano-6-methylphenyl]-1-(3-chloropyridin-2-yl)-3-{[5-(trifluoromethyl)-2H-tetrazol-2-yl]methyl}-1H-pyrazole-5-carboxamide (known from WO 2010/069502), (1E)-N-[(6-chloropyridin-3-yl)methyl]-N′-cyano-N-(2,2-difluoroethyl)ethanimideamide (known from WO 2008/009360), N-[2-(5-amino-1,3,4-thiadiazol-2-yl)-4-chloro-6-methylphenyl]-3-bromo-1-(3-chloropyridin-2-yl)-1H-pyrazole-5-carboxamide (known from CN 102057925), methyl 2-[3,5-dibromo-2-({[3-bromo-1-(3-chloropyridin-2-yl)-1H-pyrazol-5-yl]carbonyl}amino)benzoyl]-2-ethyl-1-methylhydrazinecarboxylate (known from WO 2011/049233), heptafluthrin, pyriminostrobin, flufenoxystrobin and 3-chloro-N2-(2-cyanopropan-2-yl)-N1-[4-(1,1,1,2,3,3,3-heptafluoropropan-2-yl)-2-methylphenyl]phthalamide (known from WO2012/034472).

Fungicides suitable as mixing partners are:

(1) Ergosterol biosynthesis inhibitors, for example (1.1) aldimorph, (1.2) azaconazole, (1.3) bitertanol, (1.4) bromuconazole, (1.5) cyproconazole, (1.6) diclobutrazole, (1.7) difenoconazole, (1.8) diniconazole, (1.9) diniconazole-M, (1.10) dodemorph, (1.11) dodemorph acetate, (1.12) epoxiconazole, (1.13) etaconazole, (1.14) fenarimol, (1.15) fenbuconazole, (1.16) fenhexamid, (1.17) fenpropidin, (1.18) fenpropimorph, (1.19) fluquinconazole, (1.20) flurprimidol, (1.21) flusilazole, (1.22) flutriafole, (1.23) furconazole, (1.24) furconazole-cis, (1.25) hexaconazole, (1.26) imazalil, (1.27) imazalil sulphate, (1.28) imibenconazole, (1.29) ipconazole, (1.30) metconazole, (1.31) myclobutanil, (1.32) naftifin, (1.33) nuarimol, (1.34) oxpoconazole, (1.35) paclobutrazole, (1.36) pefurazoate, (1.37) penconazole, (1.38) piperalin, (1.39) prochloraz, (1.40) propiconazole, (1.41) prothioconazole, (1.42) pyributicarb, (1.43) pyrifenox, (1.44) quinconazole, (1.45) simeconazole, (1.46) spiroxamine, (1.47) tebuconazole, (1.48) terbinafin, (1.49) tetraconazole, (1.50) triadimefon, (1.51) triadimenol, (1.52) tridemorph, (1.53) triflumizole, (1.54) triforine, (1.55) triticonazole, (1.56) uniconazole, (1.57) uniconazole-P, (1.58) viniconazole, (1.59) voriconazole, (1.60) 1-(4-chlorophenyl)-2-(1H-1,2,4-triazol-1-yl)cycloheptanol, (1.61) methyl 1-(2,2-dimethyl-2,3-dihydro-1H-inden-1-yl)-1H-imidazole-5-carboxylate, (1.62) N′-{5-(difluoromethyl)-2-methyl-4-[3-(trimethylsilyl)propoxy]phenyl}-N-ethyl-N-methylimidoformamide, (1.63)N-ethyl-N-methyl-N′-{2-methyl-5-(trifluoromethyl)-4-[3-(trimethylsilyl)propoxy]phenyl}imidoformamide and (1.64) O-[1-(4-methoxyphenoxy)-3,3-dimethylbutan-2-yl]-1H-imidazole-1-carbothioate, (1.65) pyrisoxazole.

(2) Respiration inhibitors (respiratory chain inhibitors), for example (2.1) bixafen, (2.2) boscalid, (2.3) carboxin, (2.4) diflumetorim, (2.5) fenfuram, (2.6) fluopyram, (2.7) flutolanil, (2.8) fluxapyroxad, (2.9) furametpyr, (2.10) furmecyclox, (2.11) isopyrazam mixture of the syn-epimeric racemate 1RS,4SR,9RS and the anti-empimeric racemate 1RS,4SR,9SR, (2.12) isopyrazam (anti-epimeric racemate), (2.13) isopyrazam (anti-epimeric enantiomer 1R,4S,9S), (2.14) isopyrazam (anti-epimeric enantiomer 1S,4R,9R), (2.15) isopyrazam (syn-epimeric racemate 1RS,4SR,9RS), (2.16) isopyrazam (syn-epimeric enantiomer 1R,4S,9R), (2.17) isopyrazam (syn-epimeric enantiomer 1S,4R,9S), (2.18) mepronil, (2.19) oxycarboxin, (2.20) penflufen, (2.21) penthiopyrad, (2.22) sedaxane, (2.23) thifluzamide, (2.24) 1-methyl-N-[2-(1,1,2,2-tetrafluoroethoxy)phenyl]-3-(trifluoromethyl)-1H-pyrazole-4-carboxamide, (2.25) 3-(difluoromethyl)-1-methyl-N-[2-(1,1,2,2-tetrafluoroethoxy)phenyl]-1H-pyrazole-4-carboxamide, (2.26) 3-(difluoromethyl)-N-[4-fluoro-2-(1,1,2,3,3,3-hexafluoropropoxy)phenyl]-1-methyl-1H-pyrazole-4-carboxamide, (2.27)N-[1-(2,4-dichlorophenyl)-1-methoxypropan-2-yl]-3-(difluoromethyl)-1-methyl-1H-pyrazole-4-carboxamide, (2.28) 5,8-difluoro-N-[2-(2-fluoro-4-{[4-(trifluoromethyl)pyridin-2-yl]oxy}phenyl)ethyl]quinazoline-4-amine, (2.29) benzovindiflupyr, (2.30)N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide and (2.31)N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, (2.32) 3-(difluoromethyl)-1-methyl-N-(1,1,3-trimethyl-2,3-dihydro-1H-inden-4-yl)-1H-pyrazole-4-carboxamide, (2.33) 1,3,5-trimethyl-N-(1,1,3-trimethyl-2,3-dihydro-1H-inden-4-yl)-1H-pyrazole-4-carboxamide, (2.34) 1-methyl-3-(trifluoromethyl)-N-(1,1,3-trimethyl-2,3-dihydro-1H-inden-4-yl)-1H-pyrazole-4-carboxamide, (2.35) 1-methyl-3-(trifluoromethyl)-N-[(3R)-1,1,3-trimethyl-2,3-dihydro-1H-inden-4-yl]-1H-pyrazole-4-carboxamide, (2.36) 1-methyl-3-(trifluoromethyl)-N-[(3S)-1,1,3-trimethyl-2,3-dihydro-1H-inden-4-yl]-1H-pyrazole-4-carboxamide, (2.37) 3-(difluoromethyl)-1-methyl-N-[(3S)-1,1,3-trimethyl-2,3-dihydro-1H-inden-4-yl]-1H-pyrazole-4-carboxamide, (2.38) 3-(difluoromethyl)-1-methyl-N-[(3R)-1,1,3-trimethyl-2,3-dihydro-1H-inden-4-yl]-1H-pyrazole-4-carboxamide, (2.39) 1,3,5-trimethyl-N-[(3R)-1,1,3-trimethyl-2,3-dihydro-1H-inden-4-yl]-1H-pyrazole-4-carboxamide, (2.40) 1,3,5-trimethyl-N-[(3S)-1,1,3-trimethyl-2,3-dihydro-1H-inden-4-yl]-1H-pyrazole-4-carboxamide, (2.41) benodanil, (2.42) 2-chloro-N-(1,1,3-trimethyl-2,3-dihydro-1H-inden-4-yl)pyridine-3-carboxamide, (2.43) isofetamid

(3) Respiration inhibitors (respiratory chain inhibitors) acting on complex III of the respiratory chain, for example (3.1) ametoctradin, (3.2) amisulbrom, (3.3) azoxystrobin, (3.4) cyazofamid, (3.5) coumethoxystrobin, (3.6) coumoxystrobin, (3.5) dimoxystrobin, (3.8) enestroburin, (3.9) famoxadone, (3.10) fenamidone, (3.11) flufenoxystrobin, (3.12) fluoxastrobin, (3.13) kresoxim-methyl, (3.14) metominostrobin, (3.15) orysastrobin, (3.16) picoxystrobin, (3.17) pyraclostrobin, (3.18) pyrametostrobin, (3.19) pyraoxystrobin, (3.20) pyribencarb, (3.21) triclopyricarb, (3.22) trifloxystrobin, (3.23) (2E)-2-(2-{[6-(3-chloro-2-methylphenoxy)-5-fluoropyrimidin-4-yl]oxy}phenyl)-2-(methoxyimino)-N-methylethanamide, (3.24) (2E)-2-(methoxyimino)-N-methyl-2-(2-{[({(1E)-1-[3-(trifluoromethyl)phenyl]ethylidene}amino)oxy]methyl}phenyl)ethanamide, (3.25) (2E)-2-(methoxyimino)-N-methyl-2-{2-[(E)-({1-[3-(trifluoromethyl)phenyl]ethoxy}imino)methyl]phenyl}ethanamide, (3.26) (2E)-2-{2-[({[(1E)-1-(3-{[(E)-1-fluoro-2-phenylethenyl]oxy}phenyl)ethylidene]amino}oxy)methyl]phenyl}-2-(methoxyimino)-N-methylethanamide, (3.27) (2E)-2-{2-[({[(2E,3E)-4-(2,6-dichlorophenyl)but-3-en-2-ylidene]amino}oxy)methyl]phenyl}-2-(methoxyimino)-N-methylethanamide, (3.28) 2-chloro-N-(1,1,3-trimethyl-2,3-dihydro-1H-inden-4-yl)pyridine-3-carboxamide, (3.29) 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, (3.30) methyl (2E)-2-{2-[({cyclopropyl[(4-methoxyphenyl)imino]methyl}sulphanyl)methyl]phenyl}-3-methoxyprop-2-enoate, (3.31)N-(3-ethyl-3,5,5-trimethylcyclohexyl)-3-(formylamino)-2-hydroxybenzamide, (3.32) 2-{2-[(2,5-dimethylphenoxy)methyl]phenyl}-2-methoxy-N-methylacetamide. (4) Mitosis and cell division inhibitors, for example (4.1) benomyl, (4.2) carbendazim, (4.3) chlorfenazole, (4.4) diethofencarb, (4.5) ethaboxam, (4.6) fluopicolid, (4.7) fuberidazole, (4.8) pencycuron, (4.9) thiabendazole, (4.10) thiophanate-methyl, (4.11) thiophanate, (4.12) zoxamide, (4.13) 5-chloro-7-(4-methylpiperidin-1-yl)-6-(2,4,6-trifluorophenyl) [1,2,4]triazolo[1,5-a]-pyrimidine and (4.14) 3-chloro-5-(6-chloropyridin-3-yl)-6-methyl-4-(2,4,6-trifluorophenyl)pyridazine.

(5) Compounds having multisite activity, for example (5.1) Bordeaux mixture, (5.2) captafol, (5.3) captan, (5.4) chlorothalonil, (5.5) copper preparations such as copper hydroxide, (5.6) copper naphthenate, (5.7) copper oxide, (5.8) copper oxychloride, (5.9) copper sulphate, (5.10) dichlofluanid, (5.11) dithianon, (5.12) dodine, (5.13) dodine free base, (5.14) ferbam, (5.15) fluorfolpet, (5.16) folpet, (5.17) guazatine, (5.18) guazatine acetate, (5.19) iminoctadine, (5.20) iminoctadine albesilate, (5.21) iminoctadine triacetate, (5.22) mancopper, (5.23) mancozeb, (5.24) maneb, (5.25) metiram, (5.26) zinc metiram, (5.27) copper-oxine, (5.28) propamidine, (5.29) propineb, (5.30) sulphur and sulphur preparations, for example calcium polysulphide, (5.31) thiram, (5.32) tolylfluanid, (5.33) zineb, (5.34) ziram and (5.35) anilazine.

(6) Resistance inducers, for example (6.1) acibenzolar-S-methyl, (6.2) isotianil, (6.3) probenazole, (6.4) tiadinil and (6.5) laminarin.

(7) Amino acid and protein biosynthesis inhibitors, for example (7.1), (7.2) blasticidin-S, (7.3) cyprodinil, (7.4) kasugamycin, (7.5) kasugamycin hydrochloride hydrate, (7.6) mepanipyrim, (7.7) pyrimethanil, (7.8) 3-(5-fluoro-3,3,4,4-tetramethyl-3,4-dihydroisoquinolin-1-yl)quinoline and (7.9) oxytetracycline and (7.10) streptomycin.

(8) ATP production inhibitors, for example (8.1) fentin acetate, (8.2) fentin chloride, (8.3) fentin hydroxide and (8.4) silthiofam.

(9) Cell wall synthesis inhibitors, for example (9.1) benthiavalicarb, (9.2) dimethomorph, (9.3) flumorph, (9.4) iprovalicarb, (9.5) mandipropamid, (9.6) polyoxins, (9.7) polyoxorim, (9.8) validamycin A, (9.9) valifenalate and (9.10) polyoxin B.

(10) Lipid and membrane synthesis inhibitors, for example (10.1) biphenyl, (10.2) chlorneb, (10.3) dicloran, (10.4) edifenphos, (10.5) etridiazole, (10.6) iodocarb, (10.7) iprobenfos, (10.8) isoprothiolane, (10.9) propamocarb, (10.10) propamocarb hydrochloride, (10.11) prothiocarb, (10.12) pyrazophos, (10.13) quintozene, (10.14) tecnazene and (10.15) tolclofos-methyl.

(11) Melanin biosynthesis inhibitors, for example (11.1) carpropamid, (11.2) diclocymet, (11.3) fenoxanil, (11.4) fthalide, (11.5) pyroquilon, (11.6) tricyclazole and (11.7) 2,2,2-trifluoroethyl {3-methyl-1-[(4-methylbenzoyl)amino]butan-2-yl}carbamate.

(12) Nucleic acid synthesis inhibitors, for example (12.1) benalaxyl, (12.2) benalaxyl-M (kiralaxyl), (12.3) bupirimate, (12.4) clozylacon, (12.5) dimethirimol, (12.6) ethirimol, (12.7) furalaxyl, (12.8) hymexazole, (12.9) metalaxyl, (12.10) metalaxyl-M (mefenoxam), (12.11) ofurace, (12.12) oxadixyl, (12.13) oxolinic acid and (12.14) octhilinone.

(13) Signal transduction inhibitors, for example (13.1) chlozolinate, (13.2) fenpiclonil, (13.3) fludioxonil, (13.4) iprodione, (13.5) procymidone, (13.6) quinoxyfen, (13.7) vinclozolin and (13.8) proquinazid.

(14) Decouplers, for example (14.1) binapacryl, (14.2) dinocap, (14.3) ferimzone, (14.4) fluazinam and (14.5) meptyldinocap.

(15) Further compounds, for example (15.1) benthiazole, (15.2) bethoxazine, (15.3) capsimycin, (15.4) carvone, (15.5) chinomethionat, (15.6) pyriofenone (chlazafenone), (15.7) cufraneb, (15.8) cyflufenamid, (15.9) cymoxanil, (15.10) cyprosulfamide, (15.11) dazomet, (15.12) debacarb, (15.13) dichlorophen, (15.14) diclomezine, (15.15) difenzoquat, (15.16) difenzoquat methylsulphate, (15.17) diphenylamine, (15.18) EcoMate, (15.19) fenpyrazamine, (15.20) flumetover, (15.21) fluorimid, (15.22) flusulfamide, (15.23) flutianil, (15.24) fosetyl-aluminium, (15.25) fosetyl-calcium, (15.26) fosetyl-sodium, (15.27) hexachlorobenzene, (15.28) irumamycin, (15.29) methasulfocarb, (15.30) methyl isothiocyanate, (15.31) metrafenone, (15.32) mildiomycin, (15.33) natamycin, (15.34) nickel dimethyldithiocarbamate, (15.35) nitrothal-isopropyl, (15.36) octhilinone, (15.37) oxamocarb, (15.38) oxyfenthiin, (15.39) pentachlorophenol and its salts, (15.40) phenothrin, (15.41) phosphoric acid and its salts, (15.42) propamocarb-fosetylate, (15.43) propanosine-sodium, (15.44) pyrimorph, (15.45) (2E)-3-(4-tert-butylphenyl)-3-(2-chloropyridin-4-yl)-1-(morpholin-4-yl)prop-2-en-1-one, (15.46) (2Z)-3-(4-tert-butylphenyl)-3-(2-chloropyridin-4-yl)-1-(morpholin-4-yl)prop-2-en-1-one, (15.47) pyrrolnitrin, (15.48) tebufloquin, (15.49) tecloftalam, (15.50) tolnifanide, (15.51) triazoxide, (15.52) trichlamide, (15.53) zarilamid, (15.54) (3S,6 S,7R,8R)-8-benzyl-3-[{3-[(isobutyryloxy)methoxy]-4-methoxypyridin-2-yl}carbonyl)amino]-6-methyl-4,9-dioxo-1,5-dioxonan-7-yl 2-methylpropanoate, (15.55) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.56) 1-(4-{4-[(55)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.57) 1-(4-{4-[5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.58) 1-(4-methoxyphenoxy)-3,3-dimethylbutan-2-yl 1H-imidazole-1-carboxylate, (15.59) 2,3,5,6-tetrachloro-4-(methylsulphonyl)pyridine, (15.60) 2,3-dibutyl-6-chlorothieno[2,3-d]pyrimidin-4(3H)-one, (15.61) 2,6-dimethyl-1H,5H-[1,4]dithiino[2,3-c:5,6-c]dipyrrole-1,3,5,7(2H,6H)-tetrone, (15.62) 2-[5-methyl-3-(trifluoromethyl)-1H-pyrazol-1-yl]-1-(4-{4-[(5R)-5-phenyl-4,5-dihydro-1,2-oxazol-3-yl]-1,3-thiazol-2-yl}piperidin-1-yl)ethanone, (15.63) 2-[5-methyl-3-(trifluoromethyl)-1H-pyrazol-1-yl]-1-(4-{4-[(5S)-5-phenyl-4,5-dihydro-1,2-oxazol-3-yl]-1,3-thiazol-2-yl}piperidin-1-yl)ethanone, (15.64) 2-[5-methyl-3-(trifluoromethyl)-1H-pyrazol-1-yl]-1-{4-[4-(5-phenyl-4,5-dihydro-1,2-oxazol-3-yl)-1,3-thiazol-2-yl]piperidin-1-yl}ethanone, (15.65) 2-butoxy-6-iodo-3-propyl-4H-chromen-4-one, (15.66) 2-chloro-5-[2-chloro-1-(2,6-difluoro-4-methoxyphenyl)-4-methyl-1H-imidazol-5-yl]pyridine, (15.67) 2-phenylphenol and salts, (15.68) 3-(4,4,5-trifluoro-3,3-dimethyl-3,4-dihydroisoquinolin-1-yl)quinoline, (15.69) 3,4,5-trichloropyridine-2,6-dicarbonitrile, (15.70) 3-chloro-5-(4-chlorophenyl)-4-(2,6-difluorophenyl)-6-methylpyridazine, (15.71) 4-(4-chlorophenyl)-5-(2,6-difluorophenyl)-3,6-dimethylpyridazine, (15.72) 5-amino-1,3,4-thiadiazole-2-thiol, (15.73) 5-chloro-N′-phenyl-N′-(prop-2-yn-1-yl)thiophene-2-sulphonohydrazide, (15.74) 5-fluoro-2-[(4-fluorobenzyl)oxy]pyrimidine-4-amine, (15.75) 5-fluoro-2-[(4-methylbenzyl)oxy]pyrimidine-4-amine, (15.76) 5-methyl-6-octyl[1,2,4]triazolo[1,5-a]pyrimidine-7-amine, (15.77) ethyl (2Z)-3-amino-2-cyano-3-phenylacrylate, (15.78) N′-(4-[3-(4-chlorobenzyl)-1,2,4-thiadiazol-5-yl]oxyl-2,5-dimethylphenyl)-N-ethyl-N-methylimidoformamide, (15.79)N-(4-chlorobenzyl)-3-[3-methoxy-4-(prop-2-yn-1-yloxy)phenyl]propanamide, (15.80)N-[(4-chlorophenyl)(cyano)methyl]-3-[3-methoxy-4-(prop-2-yn-1-yloxy)phenyl]propanamide, (15.81)N-[(5-bromo-3-chloropyridin-2-yl)methyl]-2,4-dichloronicotinamide, (15.82)N-[1-(5-bromo-3-chloropyridin-2-yl)ethyl]-2,4-dichloronicotinamide, (15.83)N-[1-(5-bromo-3-chloropyridin-2-yl)ethyl]-2-fluoro-4-iodonicotinamide, (15.84) N-{(E)-[(cyclopropylmethoxy)imino][6-(difluoromethoxy)-2,3-difluorophenyl]methyl}-2-phenylacetamide, (15.85) N—{(Z)-[(cyclopropylmethoxy)imino][6-(difluoromethoxy)-2,3-difluorophenyl]methyl}-2-phenylacetamide, (15.86) N′-{4-[(3-tert-butyl-4-cyano-1,2-thiazol-5-yl)oxy]-2-chloro-5-methylphenyl}-N-ethyl-N-methylimidoformamide, (15.87)N-methyl-2-(1-{[5-methyl-3-(trifluoromethyl)-1H-pyrazol-1-yl]acetyl}piperidin-4-yl)-N-(1,2,3,4-tetrahydronaphthalen-1-yl)-1,3-thiazole-4-carboxamide, (15.88) N-methyl-2-(1-{[5-methyl-3-(trifluoromethyl)-1H-pyrazol-1-yl]acetyl}piperidin-4-yl)-N-[(1R)-1,2,3,4-tetrahydronaphthalen-1-yl]-1,3-thiazole-4-carboxamide, (15.89)N-methyl-2-(1-{[5-methyl-3-(trifluoromethyl)-1H-pyrazol-1-yl]acetyl}piperidin-4-yl)-N-[(1S)-1,2,3,4-tetrahydronaphthalen-1-yl]-1,3-thiazole-4-carboxamide, (15.90) pentyl {6-[({[(1-methyl-1H-tetrazol-5-yl)(phenyl)methylene]amino}oxy)methyl]pyridin-2-yl}carbamate, (15.91) phenazine-1-carboxylic acid, (15.92) quinolin-8-ol, (15.93) quinolin-8-ol sulphate (2:1), (15.94) tert-butyl {6-[({[(1-methyl-1H-tetrazol-5-yl)(phenyl)methylene]amino}oxy)methyl]pyridin-2-yl}carbamate, (15.95) 1-methyl-3-(trifluoromethyl)-N-[2′-(trifluoromethyl)biphenyl-2-yl]-1H-pyrazole-4-carboxamide, (15.96)N-(4′-chlorobiphenyl-2-yl)-3-(difluoromethyl)-1-methyl-1H-pyrazole-4-carboxamide, (15.97)N-(2′,4′-dichlorobiphenyl-2-yl)-3-(difluoromethyl)-1-methyl-1H-pyrazole-4-carboxamide, (15.98) 3-(difluoromethyl)-1-methyl-N-[4′-(trifluoromethyl)biphenyl-2-yl]-1H-pyrazole-4-carboxamide, (15.99) N-(2′,5′-difluorobiphenyl-2-yl)-1-methyl-3-(trifluoromethyl)-1H-pyrazole-4-carboxamide, (15.100) 3-(difluoromethyl)-1-methyl-N-[4′-(prop-1-yn-1-yl)biphenyl-2-yl]-1H-pyrazole-4-carboxamide, (15.101) 5-fluoro-1,3-dimethyl-N-[4′-(prop-1-yn-1-yl)biphenyl-2-yl]-1H-pyrazole-4-carboxamide, (15.102) 2-chloro-N-[4′-(prop-1-yn-1-yl)biphenyl-2-yl]nicotinamide, (15.103) 3-(difluoromethyl)-N-[4′-(3,3-dimethylbut-1-yn-1-yl)biphenyl-2-yl]-1-methyl-1H-pyrazole-4-carboxamide, (15.104)N-[4′-(3,3-dimethylbut-1-yn-1-yl)biphenyl-2-yl]-5-fluoro-1,3-dimethyl-1H-pyrazole-4-carboxamide, (15.105) 3-(difluoromethyl)-N-(4′-ethynylbiphenyl-2-yl)-1-methyl-1H-pyrazole-4-carboxamide, (15.106)N-(4′-ethynylbiphenyl-2-yl)-5-fluoro-1,3-dimethyl-1H-pyrazole-4-carboxamide, (15.107) 2-chloro-N-(4′-ethynylbiphenyl-2-yl)nicotinamide, (15.108) 2-chloro-N-[4′-(3,3-dimethylbut-1-yn-1-yl)biphenyl-2-yl]nicotinamide, (15.109) 4-(difluoromethyl)-2-methyl-N-[4′-(trifluoromethyl)biphenyl-2-yl]-1,3-thiazole-5-carboxamide, (15.110) 5-fluoro-N-[4′-(3-hydroxy-3-methylbut-1-yn-1-yl)biphenyl-2-yl]-1,3-dimethyl-1H-pyrazole-4-carboxamide, (15.111) 2-chloro-N-[4′-(3-hydroxy-3-methylbut-1-yn-1-yl)biphenyl-2-yl]nicotinamide, (15.112) 3-(difluoromethyl)-N-[4′-(3-methoxy-3-methylbut-1-yn-1-yl)biphenyl-2-yl]-1-methyl-1H-pyrazole-4-carboxamide, (15.113) 5-fluoro-N-[4′-(3-methoxy-3-methylbut-1-yn-1-yl)biphenyl-2-yl]-1,3-dimethyl-1H-pyrazole-4-carboxamide, (15.114) 2-chloro-N-[4′-(3-methoxy-3-methylbut-1-yn-1-yl)biphenyl-2-yl]nicotinamide, (15.115) (5-bromo-2-methoxy-4-methylpyridin-3-yl)(2,3,4-trimethoxy-6-methylphenyl)methanone, (15.116)N-[2-(4-[3-(4-chlorophenyl)prop-2-yn-1-yl]oxy-3-methoxyphenyl)ethyl]-N2-(methylsulphonyl)valinamide, (15.117) 4-oxo-4-[(2-phenylethyl)amino]butanoic acid, (15.118) but-3-yn-1-yl {6-[({[(Z)-(1-methyl-1H-tetrazol-5-yl)(phenyl)methylene]amino}oxy)methyl]pyridin-2-yl}carbamate, (15.119) 4-amino-5-fluoropyrimidin-2-ol (tautomeric form: 4-amino-5-fluoropyrimidin-2(1H)-one), (15.120) propyl 3,4,5-trihydroxybenzoate, (15.121) 1,3-dimethyl-N-(1,1,3-trimethyl-2,3-dihydro-1H-inden-4-yl)-1H-pyrazole-4-carboxamide, (15.122) 1,3-dimethyl-N-[(3R)-1,1,3-trimethyl-2,3-dihydro-1H-inden-4-yl]-1H-pyrazole-4-carboxamide, (15.123) 1,3-dimethyl-N-[(3S)-1,1,3-trimethyl-2,3-dihydro-1H-inden-4-yl]-1H-pyrazole-4-carboxamide, (15.124) [3-(4-chloro-2-fluorophenyl)-5-(2,4-difluorophenyl)-1,2-oxazol-4-yl](pyridin-3-yl)methanol, (15.125) (S)-[3-(4-chloro-2-fluorophenyl)-5-(2,4-difluorophenyl)-1,2-oxazol-4-yl](pyridin-3-yl)methanol, (15.126) (R)-[3-(4-chloro-2-fluorophenyl)-5-(2,4-difluorophenyl)-1,2-oxazol-4-yl](pyridin-3-yl)methanol, (15.127) 2-{[3-(2-chlorophenyl)-2-(2,4-difluorophenyl)oxiran-2-yl]methyl}-2,4-dihydro-3H-1,2,4-triazole-3-thione, (15.128) 1-{[3-(2-chlorophenyl)-2-(2,4-difluorophenyl)oxiran-2-yl]methyl}-1H-1,2,4-triazol-5-yl thiocyanate, (15.129) 5-(allylsulphanyl)-1-{[3-(2-chlorophenyl)-2-(2,4-difluorophenyl)oxiran-2-yl]methyl}-1H-1,2,4-triazole, (15.130) 2-[1-(2,4-dichlorophenyl)-5-hydroxy-2,6,6-trimethylheptan-4-yl]-2,4-dihydro-3H-1,2,4-triazole-3-thione, (15.131) 2-{[rel(2R,3S)-3-(2-chlorophenyl)-2-(2,4-difluorophenyl)oxiran-2-yl]methyl}-2,4-dihydro-3H-1,2,4-triazole-3-thione, (15.132) 2-{[rel(2R,3R)-3-(2-chlorophenyl)-2-(2,4-difluorophenyl)oxiran-2-yl]methyl}-2,4-dihydro-3H-1,2,4-triazole-3-thione, (15.133) 1-{[rel(2R,3S)-3-(2-chlorophenyl)-2-(2,4-difluorophenyl)oxiran-2-yl]methyl}-1H-1,2,4-triazol-5-yl thiocyanate, (15.134) 1-{[rel(2R,3R)-3-(2-chlorophenyl)-2-(2,4-difluorophenyl)oxiran-2-yl]methyl}-1H-1,2,4-triazol-5-yl thiocyanate, (15.135) 5-(allylsulphanyl)-1-{[rel(2R,3S)-3-(2-chlorophenyl)-2-(2,4-difluorophenyl)oxiran-2-yl]methyl}-1H-1,2,4-triazole, (15.136) 5-(allylsulphanyl)-1-{[rel(2R,3R)-3-(2-chlorophenyl)-2-(2,4-difluorophenyl)oxiran-2-yl]methyl}-1H-1,2,4-triazole, (15.137) 2-[(2S,4S,5S)-1-(2,4-dichlorophenyl)-5-hydroxy-2,6,6-trimethylheptan-4-yl]-2,4-dihydro-3H-1,2,4-triazole-3-thione, (15.138) 2-[(2R,4S,5S)-1-(2,4-dichlorophenyl)-5-hydroxy-2,6,6-trimethylheptan-4-yl]-2,4-dihydro-3H-1,2,4-triazole-3-thione, (15.139) 2-[(2R,4R,5R)-1-(2,4-dichlorophenyl)-5-hydroxy-2,6,6-trimethylheptan-4-yl]-2,4-dihydro-3H-1,2,4-triazole-3-thione, (15.140) 2-[(2S,4R,5R)-1-(2,4-dichlorophenyl)-5-hydroxy-2,6,6-trimethylheptan-4-yl]-2,4-dihydro-3H-1,2,4-triazole-3-thione, (15.141) 2-[(2S,4S,5R)-1-(2,4-dichlorophenyl)-5-hydroxy-2,6,6-trimethylheptan-4-yl]-2,4-dihydro-3H-1,2,4-triazole-3-thione, (15.142) 2-[(2R,4S,5R)-1-(2,4-dichlorophenyl)-5-hydroxy-2,6,6-trimethylheptan-4-yl]-2,4-dihydro-3H-1,2,4-triazole-3-thione, (15.143) 2-[(2R,4R,5S)-1-(2,4-dichlorophenyl)-5-hydroxy-2,6,6-trimethylheptan-4-yl]-2,4-dihydro-3H-1,2,4-triazole-3-thione, (15.144) 2-[(2S,4R,5S)-1-(2,4-dichlorophenyl)-5-hydroxy-2,6,6-trimethylheptan-4-yl]-2,4-dihydro-3H-1,2,4-triazole-3-thione, (15.145) 2-fluoro-6-(trifluoromethyl)-N-(1,1,3-trimethyl-2,3-dihydro-1H-inden-4-yl)benzamide, (15.146) 2-(6-benzylpyridin-2-yl)quinazoline, (15.147) 2-[6-(3-fluoro-4-methoxyphenyl)-5-methylpyridin-2-yl]quinazoline, (15.148) 3-(4,4-difluoro-3,3-dimethyl-3,4-dihydroisoquinolin-1-yl)quinoline, (15.149) abscisic acid, (15.150) 3-(difluoromethyl)-N-methoxy-1-methyl-N-[1-(2,4,6-trichlorophenyl)propan-2-yl]-1H-pyrazole-4-carboxamide, (15.151) N′-[5-bromo-6-(2,3-dihydro-1H-inden-2-yloxy)-2-methylpyridin-3-yl]-N-ethyl-N-methylimidoformamide, (15.152) N′-{5-bromo-6-[1-(3,5-difluorophenyl)ethoxy]-2-methylpyridin-3-yl}-N-ethyl-N-methylimidoformamide, (15.153) N′-{5-bromo-6-[(1R)-1-(3,5-difluorophenyl)ethoxy]-2-methylpyridin-3-yl}-N-ethyl-N-methylimidoformamide, (15.154) N′-{5-bromo-6-[(1S)-1-(3,5-difluorophenyl)ethoxy]-2-methylpyridin-3-yl}-N-ethyl-N-methylimidoformamide, (15.155) N′-{5-bromo-6-[(cis-4-isopropylcyclohexyl)oxy]-2-methylpyridin-3-yl}-N-ethyl-N-methylimidoformamide, (15.156) N′-{5-bromo-6-[(trans-4-isopropylcyclohexyl)oxy]-2-methylpyridin-3-yl}-N-ethyl-N-methylimidoformamide, (15.157)N-cyclopropyl-3-(difluoromethyl)-5-fluoro-N-(2-isopropylbenzyl)-1-methyl-1H-pyrazole-4-carboxamide, (15.158)N-cyclopropyl-N-(2-cyclopropylbenzyl)-3-(difluoromethyl)-5-fluoro-1-methyl-1H-pyrazole-4-carboxamide, (15.159)N-(2-tert-butylbenzyl)-N-cyclopropyl-3-(difluoromethyl)-5-fluoro-1-methyl-1H-pyrazole-4-carboxamide, (15.160)N-(5-chloro-2-ethylbenzyl)-N-cyclopropyl-3-(difluoromethyl)-5-fluoro-1-methyl-1H-pyrazole-4-carboxamide, (15.161)N-(5-chloro-2-isopropylbenzyl)-N-cyclopropyl-3-(difluoromethyl)-5-fluoro-1-methyl-1H-pyrazole-4-carboxamide, (15.162)N-cyclopropyl-3-(difluoromethyl)-N-(2-ethyl-5-fluorobenzyl)-5-fluoro-1-methyl-1H-pyrazole-4-carboxamide, (15.163) N-cyclopropyl-3-(difluoromethyl)-5-fluoro-N-(5-fluoro-2-isopropylbenzyl)-1-methyl-1H-pyrazole-4-carboxamide, (15.164)N-cyclopropyl-N-(2-cyclopropyl-5-fluorobenzyl)-3-(difluoromethyl)-5-fluoro-1-methyl-1H-pyrazole-4-carboxamide, (15.165)N-(2-cyclopentyl-5-fluorobenzyl)-N-cyclopropyl-3-(difluoromethyl)-5-fluoro-1-methyl-1H-pyrazole-4-carboxamide, (15.166)N-cyclopropyl-3-(difluoromethyl)-5-fluoro-N-(2-fluoro-6-isopropylbenzyl)-1-methyl-1H-pyrazole-4-carboxamide, (15.167)N-cyclopropyl-3-(difluoromethyl)-N-(2-ethyl-5-methylbenzyl)-5-fluoro-1-methyl-1H-pyrazole-4-carboxamide, (15.168)N-cyclopropyl-3-(difluoromethyl)-5-fluoro-N-(2-isopropyl-5-methylbenzyl)-1-methyl-1H-pyrazole-4-carboxamide, (15.169)N-cyclopropyl-N-(2-cyclopropyl-5-methylbenzyl)-3-(difluoromethyl)-5-fluoro-1-methyl-1H-pyrazole-4-carboxamide, (15.170)N-(2-tert-butyl-5-methylbenzyl)-N-cyclopropyl-3-(difluoromethyl)-5-fluoro-1-methyl-1H-pyrazole-4-carboxamide, (15.171)N-[5-chloro-2-(trifluoromethyl)benzyl]-N-cyclopropyl-3-(difluoromethyl)-5-fluoro-1-methyl-1H-pyrazole-4-carboxamide, (15.172)N-cyclopropyl-3-(difluoromethyl)-5-fluoro-1-methyl-N-[5-methyl-2-(trifluoromethyl)benzyl]-1H-pyrazole-4-carboxamide, (15.173)N-[2-chloro-6-(trifluoromethyl)benzyl]-N-cyclopropyl-3-(difluoromethyl)-5-fluoro-1-methyl-1H-pyrazole-4-carboxamide, (15.174)N-[3-chloro-2-fluoro-6-(trifluoromethyl)benzyl]-N-cyclopropyl-3-(difluoromethyl)-5-fluoro-1-methyl-1H-pyrazole-4-carboxamide, (15.175)N-cyclopropyl-3-(difluoromethyl)-N-(2-ethyl-4,5-dimethylbenzyl)-5-fluoro-1-methyl-1H-pyrazole-4-carboxamide, (15.176)N-cyclopropyl-3-(difluoromethyl)-5-fluoro-N-(2-isopropylbenzyl)-1-methyl-1H-pyrazol-4-carbothioamide, (15.177) 3-(difluoromethyl)-N-(7-fluoro-1,1,3-trimethyl-2,3-dihydro-1H-inden-4-yl)-1-methyl-1H-pyrazole-4-carboxamide, (15.178) 3-(difluoromethyl)-N-[(3R)-7-fluoro-1,1,3-trimethyl-2,3-dihydro-1H-inden-4-yl]-1-methyl-1H-pyrazole-4-carboxamide, (15.179) 3-(difluoromethyl)-N-[(3S)-7-fluoro-1,1,3-trimethyl-2,3-dihydro-1H-inden-4-yl]-1-methyl-1H-pyrazole-4-carboxamide, (15.180) N′-(2,5-dimethyl-4-phenoxyphenyl)-N-ethyl-N-methylimidoformamide, (15.181) N′-{4-[(4,5-dichloro-1,3-thiazol-2-yl)oxy]-2,5-dimethylphenyl}-N-ethyl-N-methylimidoformamide, (15.182)N-(4-chloro-2,6-difluorophenyl)-4-(2-chloro-4-fluorophenyl)-1,3-dimethyl-1H-pyrazole-5-amine. All the mixing partners mentioned in classes (1) to (15) may, as the case may be, form salts with suitable bases or acids if they are capable of doing do on the basis of their functional groups.

The compounds of the formula (I) can be combined with biological pesticides.

Biological pesticides include especially bacteria, fungi, yeasts, plant extracts and products formed by microorganisms, including proteins and secondary metabolites.

Biological pesticides include bacteria such as spore-forming bacteria, root-colonizing bacteria and bacteria which act as biological insecticides, fungicides or nematicides.

Examples of such bacteria which can be used as biological pesticides are:

Bacillus amyloliquefaciens, strain FZB42 (DSM 231179), or Bacillus cereus, especially B. cereus strain CNCM 1-1562 or Bacillus firmus, strain 1-1582 (Accession number CNCM 1-1582) or Bacillus pumilus, especially strain GB34 (Accession No. ATCC 700814) and strain QST2808 (Accession No. NRRL B-30087), or Bacillus subtilis, especially strain GB03 (Accession No. ATCC SD-1397), or Bacillus subtilis strain QST713 (Accession No. NRRL B-21661) or Bacillus subtilis strain OST 30002 (Accession No. NRRL B-50421) Bacillus thuringiensis, especially B. thuringiensis subspecies israelensis (serotype H-14), strain AM65-52 (Accession No. ATCC 1276), or B. thuringiensis subsp. aizawai, especially strain ABTS-1857 (SD-1372), or B. thuringiensis subsp. kurstaki strain HD-1, or B. thuringiensis subsp. tenebrionis strain NB 176 (SD-5428), Pasteuria penetrans, Pasteuria spp. (Rotylenchulus reniformis nematode)-PR3 (Accession Number ATCC SD-5834), Streptomyces microflavus strain AQ6121 (=QRD 31.013, NRRL B-50550), Streptomyces galbus strain AQ 6047 (Accession Number NRRL 30232).

Examples of fungi and yeasts which are used or can be used as biological pesticides are:

Beauveria bassiana, especially strain ATCC 74040, Coniothyrium minitans, especially strain CON/M/91-8 (Accession No. DSM-9660), Lecanicillium spp., especially strain HRO LEC 12, Lecanicillium lecanii, (formerly known as Verticillium lecanii), especially strain KV01, Metarhizium anisopliae, especially strain F52 (DSM3884/ATCC 90448), Metschnikowia fructicola, especially strain NRRL Y-30752, Paecilomyces fumosoroseus (now: Isaria fumosorosea), especially strain IFPC 200613, or strain Apopka 97 (Accesion No. ATCC 20874), Paecilomyces lilacinus, especially P. lilacinus strain 251 (AGAL 89/030550), Talaromyces flavus, especially strain V117b, Trichoderma atroviride, especially strain SC1 (Accession Number CBS 122089), Trichoderma harzianum, especially T. harzianum rifai T39 (Accession Number CNCM I-952).

Examples of viruses which are used or can be used as biological pesticides are:

Adoxophyes orana (summer fruit tortrix) granulosis virus (GV), Cydia pomonella (codling moth) granulosis virus (GV), Helicoverpa armigera (cotton bollworm) nuclear polyhedrosis virus (NPV), Spodoptera exigua (beet armyworm) mNPV, Spodoptera frugiperda (fall armyworm) mNPV, Spodoptera littoralis (African cotton leafworm) NPV.

Also included are bacteria and fungi which are added as ‘inoculant’ to plants or plant parts or plant organs and which, by virtue of their particular properties, promote plant growth and plant health. Examples include:

Agrobacterium spp., Azorhizobium caulinodans, Azospirillum spp., Azotobacter spp., Bradyrhizobium spp., Burkholderia spp., especially Burkholderia cepacia (formerly known as Pseudomonas cepacia), Gigaspora spp., or Gigaspora monosporum, Glomus spp., Laccaria spp., Lactobacillus buchneri, Paraglomus spp., Pisolithus tinctorus, Pseudomonas spp., Rhizobium spp., especially Rhizobium trifolii, Rhizopogon spp., Scleroderma spp., Suillus spp., Streptomyces spp.

Examples of plant extracts and products formed by microorganisms, including proteins and secondary metabolites, which are used or can be used as biological pesticides are:

Allium sativum, Artemisia absinthium, azadirachtin, Biokeeper WP, Cassia nigricans, Celastrus angulatus, Chenopodium anthelminticum, chitin, Armour-Zen, Dryopteris filix-mas, Equisetum arvense, Fortune Aza, Fungastop, Heads Up (Chenopodium quinoa saponin extract), Pyrethrum/Pyrethrins, Quassia amara, Quercus, Quillaja, Regalia, “Requiem™ Insecticide”, rotenone, ryania/ryanodine, Symphytum officinale, Tanacetum vulgare, thymol, Triact 70, TriCon, Tropaeulum majus, Urtica dioica, Veratrin, Viscum album, Brassicaceae extract, especially oilseed rape powder or mustard powder.

The compounds of the formula (I) can be combined with safeners, for example benoxacor, cloquintocet (-mexyl), cyometrinil, cyprosulfamide, dichlormid, fenchlorazole (-ethyl), fenclorim, flurazole, fluxofenim, furilazole, isoxadifen (-ethyl), mefenpyr (-diethyl), naphthalic anhydride, oxabetrinil, 2-methoxy-N-({4-[(methylcarbamoyl)amino]phenyl}sulphonyl)benzamide (CAS 129531-12-0), 4-(dichloroacetyl)-1-oxa-4-azaspiro[4.5]decane (CAS 71526-07-3), 2,2,5-trimethyl-3-(dichloroacetyl)-1,3-oxazolidine (CAS 52836-31-4).

When used as insecticides, the inventive active ingredients may also be present in their commercially available formulations and in the use forms, prepared from these formulations, as a mixture with synergists. Synergists are compounds which enhance the action of the active ingredients, without any need for the synergist added to be active itself.

When used as insecticides, the inventive active ingredients may also be present in their commercially available formulations and in the use forms, prepared from these formulations, as a mixture with inhibitors which reduce degradation of the active ingredient after use in the environment of the plant, on the surface of parts of plants or in plant tissues.

All plants and parts of plants can be treated in accordance with the invention. Plants are understood here to mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the transgenic plants and including the plant cultivars which are protectable and non-protectable by plant breeders' rights. Examples include the important crop plants, such as cereals (wheat, rice), maize, soya, potatoes, sugarbeet, tomatoes, peas and other vegetable species, cotton, tobacco, oilseed rape and also fruit plants (the fruits produced being apples, pears, citrus fruits and grapes). Parts of plants shall be understood to mean all above-ground and below-ground parts and organs of plants, such as shoot, leaf, flower and root, examples including leaves, needles, stems, trunks, flowers, fruit bodies, fruits and seeds, and also roots, tubers and rhizomes. Parts of plants also include harvested material and vegetative and generative propagation material, for example seedlings, tubers, rhizomes, cuttings and seeds.

As already mentioned above, it is possible to treat all plants and parts thereof in accordance with the invention. In a preferred embodiment, wild plant species and plant cultivars, or those obtained by conventional biological breeding techniques, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The terms “parts” or “parts of plants” or “plant parts” have been explained above.

More preferably, plants of the plant cultivars which are commercially available or are in use are treated in accordance with the invention. Plant cultivars are understood to mean plants having new properties (“traits”) and which have been obtained by conventional breeding, by mutagenesis or by recombinant DNA techniques. They may be cultivars, biotypes and genotypes.

The treatment of the plants and parts of plants with the inventive active ingredients, active ingredient combinations or compositions, or with those to be used in accordance with the invention, is effected directly or by action on their surroundings, habitat or storage space by the customary treatment methods, for example by dipping, spraying, atomizing, irrigating, evaporating, dusting, fogging, broadcasting, foaming, painting, spreading-on, injecting, watering (drenching), drip irrigating and, in the case of propagation material, especially in the case of seeds, also by dry seed treatment, wet seed treatment, slurry treatment, incrustation, coating with one or more coats, etc. It is also possible to deploy the active ingredients by the ultra-low volume method or to inject the active ingredient preparation or the active ingredient itself into the soil.

One preferred direct treatment of the plants is foliar application; in other words, the active ingredients, active ingredient combinations or compositions are applied to the foliage, and the frequency of treatment and the application rate may be adjusted for the infestation pressure of the particular pest.

In the case of systemically active ingredients, the active ingredients, active ingredient combinations or compositions access the plants via the root system. The treatment of the plants then takes place through the action of the active ingredients, active ingredient combinations or compositions on the habitat of the plant. This can be done, for example, by drenching, or by mixing into the soil or the nutrient solution, meaning that the locus of the plant (e.g. soil or hydroponic systems) is impregnated with a liquid form of the active ingredients, active ingredient combinations or compositions, or by soil application, i.e. the inventive active ingredients, active ingredient combinations or compositions are introduced in solid form (e.g. in the form of granules) into the locus of the plants. In the case of paddy rice crops, this can also be done by metering the invention in a solid application form (for example as granules) into a flooded paddy field.

The control of animal pests by the treatment of the seed of plants has long been known and is the subject of constant improvement. Nevertheless, the treatment of seed gives rise to a series of problems which cannot always be solved in a satisfactory manner Thus, it is desirable to develop methods for protecting the seed and the germinating plant which dispense with, or at least reduce considerably, the additional application of crop protection compositions during storage, after sowing or after emergence of the plants. It is additionally desirable to optimize the amount of active ingredient used so as to provide optimum protection for the seed and the germinating plant from attack by animal pests, but without damage to the plant itself by the active ingredient used. More particularly, methods for the treatment of seed should also take account of the intrinsic insecticidal or nematicidal properties of pest-resistant or -tolerant transgenic plants, in order to achieve optimum protection of the seed and of the germinating plant with minimum expenditure of crop protection products.

The present invention therefore also relates particularly to a method for protection of seed and germinating plants from attack by pests, by treating the seed with an inventive active ingredient or active ingredient for use in accordance with the invention. The method of the invention for protection of seed and germinating plants against attack by pests comprises a method in which the seed is treated simultaneously in one operation with an active ingredient of the formula (I) and a mixing partner. It also comprises a method in which the seed is treated at different times with an active ingredient of the formula (I) and a mixing partner.

The invention likewise relates to the use of the inventive active ingredients, or of an active ingredient combination comprising it, for treatment of seed to protect the seed and the resulting plant from animal pests.

The invention further relates to seed which has been treated with an inventive active ingredient or an active ingredient combination comprising it so as to afford protection from animal pests. The invention also relates to seed which has been treated simultaneously with an active ingredient of the formula (I) and a mixing partner. The invention further relates to seed which has been treated at different times with an active ingredient of the formula (I) and a mixing partner. In the case of seed which has been treated at different times with an active ingredient of the formula (I) and a mixing partner, the individual active ingredients in the inventive composition may be present on the seed in different layers. In this case, the layers comprising an active ingredient of the formula (I) and a mixing partner may be separated by an intermediate layer. The invention also relates to seed in which an active ingredient of the formula (I) and a mixing partner have been applied as part of a coating or as a further layer or further layers in addition to a coating.

The invention further relates to seed which, after the treatment with the inventive active ingredient or an active ingredient combination comprising it, has been subjected to a film-coating process to prevent dust abrasion in the seed.

One of the advantages of the present invention is that the particular systemic properties of the inventive compositions mean that treatment of the seed with these compositions protects not only the seed itself but also the resulting plants after emergence from animal pests. In this way, the immediate treatment of the crop at the time of sowing or shortly thereafter can be dispensed with.

A further advantage is that the treatment of the seed with the inventive active ingredient or an active ingredient combination comprising it can enhance germination and emergence of the treated seed.

It is likewise considered to be advantageous that the inventive active ingredients or active ingredient combinations comprising it can also be used in the case of transgenic seed in particular.

It should also be mentioned that the inventive active ingredients can be used in combination with signalling technology compositions, leading, for example, to better colonization by symbionts, for example rhizobia, mycorrhizae and/or endophytic bacteria, and/or to optimized nitrogen fixation.

The inventive compositions are suitable for protection of seed of any plant variety which is used in agriculture, in greenhouses, in forests or in horticulture. In particular, this includes seed of cereals (for example wheat, barley, rye, millet and oats), corn, cotton, soya beans, rice, potatoes, sunflowers, coffee, tobacco, canola, oilseed rape, beets (for example sugarbeets and fodder beets), peanuts, vegetables (for example tomatoes, cucumbers, bean, cruciferous vegetables, onions and lettuce), fruit plants, lawns and ornamental plants. Of particular significance is the treatment of the seed of cereals (such as wheat, barley, rye and oats), maize, soya beans, cotton, canola, oilseed rape and rice.

As already mentioned above, also of particular significance is the treatment of transgenic seed with an inventive active ingredient or an active ingredient combination. This involves the seed of plants which generally contain at least one heterologous gene which controls the expression of a polypeptide having insecticidal and/or nematicidal properties in particular. In this context, the heterologous genes in transgenic seed may be derived from microorganisms such as Bacillus, Rhizobium, Pseudomonas, Serratia, Trichoderma, Clavibacter, Glomus or Gliocladium. The present invention is particularly suitable for treatment of transgenic seed which comprises at least one heterologous gene originating from Bacillus sp. The heterologous gene is more preferably derived from Bacillus thuringiensis.

In the context of the present invention, active ingredient of the formula (I) is applied to the seed alone (or as an active ingredient combination) or in a suitable formulation. The seed is preferably treated in a state in which it is sufficiently stable for no damage to occur in the course of treatment. In general, the seed can be treated at any time between harvest and sowing. It is customary to use seed which has been separated from the plant and freed from cobs, shells, stalks, coats, hairs or the flesh of the fruits. For example, it is possible to use seed which has been harvested, cleaned and dried down to a moisture content of less than 15% by weight. Alternatively, it is also possible to use seed which, after drying, for example, has been treated with water and then dried again.

In general, in the treatment of the seed, it has to be ensured that the amount of the inventive composition and/or further additives applied to the seed is selected such that the germination of the seed is not impaired and the plant which arises therefrom is not damaged. This should be ensured particularly in the case of active ingredients which can exhibit phytotoxic effects at particular application rates.

The inventive compositions can be applied directly, i.e. without containing any other components and without having been diluted. In general, it is preferable to apply the compositions to the seed in the form of a suitable formulation. Suitable formulations and methods for seed treatment are known to those skilled in the art and are described, for example, in the following documents: U.S. Pat. No. 4,272,417 A, U.S. Pat. No. 4,245,432 A, U.S. Pat. No. 4,808,430 A, U.S. Pat. No. 5,876,739 A, US 2003/0176428 A1, WO 2002/080675 A1, WO 2002/028186 A2.

The active ingredients/active ingredient combinations usable in accordance with the invention can be converted to the customary seed-dressing formulations, such as solutions, emulsions, suspensions, powders, foams, slurries or other coating compositions for seed, and also ULV formulations.

These formulations are prepared in a known manner, by mixing the active ingredients/active ingredient combinations with customary additives, for example customary extenders and solvents or diluents, dyes, wetting agents, dispersants, emulsifiers, antifoams, preservatives, secondary thickeners, adhesives, gibberellins, and also water.

Useful dyes which may be present in the seed dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed dressing formulations usable in accordance with the invention are all substances which promote wetting and which are conventionally used for the formulation of agrochemically active compounds. Preference is given to using alkyl naphthalenesulphonates, such as diisopropyl or diisobutyl naphthalenesulphonates.

Useful dispersants and/or emulsifiers which may be present in the seed dressing formulations usable in accordance with the invention are all nonionic, anionic and cationic dispersants conventionally used for the formulation of active agrochemical ingredients. Usable with preference are nonionic or anionic dispersants or mixtures of nonionic or anionic dispersants. Suitable nonionic dispersants include especially ethylene oxide/propylene oxide block polymers, alkylphenol polyglycol ethers and tristryrylphenol polyglycol ethers, and the phosphated or sulphated derivatives thereof. Suitable anionic dispersants are especially lignosulphonates, polyacrylic acid salts and arylsulphonate-formaldehyde condensates.

Antifoams which may be present in the seed dressing formulations usable in accordance with the invention are all foam-inhibiting substances conventionally used for the formulation of active agrochemical ingredients. Preference is given to using silicone antifoams and magnesium stearate.

Preservatives which may be present in the seed dressing formulations usable in accordance with the invention are all substances usable for such purposes in agrochemical compositions. Examples include dichlorophene and benzyl alcohol hemiformal.

Secondary thickeners which may be present in the seed dressing formulations usable in accordance with the invention are all substances usable for such purposes in agrochemical compositions. Preferred examples include cellulose derivatives, acrylic acid derivatives, xanthan, modified clays and finely divided silica.

Adhesives which may be present in the seed dressing formulations usable in accordance with the invention are all customary binders usable in seed dressing products. Preferred examples include polyvinylpyrrolidone, polyvinyl acetate, polyvinyl alcohol and tylose.

The gibberellins which may be present in the seed dressing formulations usable in accordance with the invention may preferably be gibberellins A1, A3 (=gibberellic acid), A4 and A7; particular preference is given to using gibberellic acid. The gibberellins are known (cf. R. Wegler “Chemie der Pflanzenschutz- and Schadlingsbekampfungsmittel” [Chemistry of the Crop Protection Compositions and Pesticides], vol. 2, Springer Verlag, 1970, p. 401).

The seed dressing formulations usable in accordance with the invention can be used to treat a wide variety of different kinds of seed either directly or after prior dilution with water. For instance, the concentrates or the preparations obtainable therefrom by dilution with water can be used to dress the seed of cereals, such as wheat, barley, rye, oats, and triticale, and also the seed of maize, rice, oilseed rape, peas, beans, cotton, sunflowers, and beets, or else a wide variety of different vegetable seed. The seed dressing formulations usable in accordance with the invention, or the dilute preparations thereof, can also be used to dress seed of transgenic plants. In this case, additional synergistic effects may also occur in interaction with the substances formed by expression.

For treatment of seed with the seed dressing formulations usable in accordance with the invention, or the preparations prepared therefrom by adding water, all mixing units usable customarily for the seed dressing are useful. Specifically, the procedure in the seed dressing is to place the seed into a mixer, to add the particular desired amount of seed dressing formulations, either as such or after prior dilution with water, and to mix everything until the formulation is distributed homogeneously on the seed. If appropriate, this is followed by a drying operation.

The application rate of the seed dressing formulations usable in accordance with the invention can be varied within a relatively wide range. It is guided by the particular content of the active ingredient in the formulations and by the seed. The application rates in the case of active ingredients/active ingredient combinations are generally between 0.001 and 50 g per kilogram of seed, preferably between 0.01 and 15 g per kilogram of seed.

Preferred plants are those from the group of the useful plants, ornamental plants, turfgrass types, commonly used trees which are employed as ornamentals in public and domestic areas, and forestry trees. Forestry trees include trees for the production of timber, cellulose, paper and products made from parts of the trees.

The term “useful plants” as used here refers to crop plants which are employed as plants for obtaining foods, animal feeds, fuels or for industrial purposes.

The useful plants include, for example, the following types of plants: turf, vines, cereals, for example wheat, barley, rye, oats, rice, maize and millet/sorghum; beet, for example sugar beet and fodder beet; fruits, for example pome fruit, stone fruit and soft fruit, for example apples, pears, plums, peaches, almonds, cherries and berries, for example strawberries, raspberries, blackberries; legumes, for example beans, lentils, peas and soya beans; oil crops, for example oilseed rape, mustard, poppies, olives, sunflowers, coconuts, castor oil plants, cocoa beans and peanuts; cucurbits, for example pumpkin/squash, cucumbers and melons; fibre plants, for example cotton, flax, hemp and jute; citrus fruit, for example, oranges, lemons, grapefruit and tangerines; vegetables, for example spinach, lettuce, asparagus, cabbage species, carrots, onions, tomatoes, potatoes and bell peppers; Lauraceae, for example avocado, Cinnamomum, camphor, or also plants such as tobacco, nuts, coffee, aubergine, sugarcane, tea, pepper, grapevines, hops, bananas, latex plants and ornamentals, for example flowers, shrubs, deciduous trees and coniferous trees. This enumeration does not constitute a limitation.

Particularly suitable target crops are considered to be the following plants: cotton, aubergine, turf, pome fruit, stone fruit, soft fruit, maize, wheat, barley, cucumber, tobacco, vines, rice, cereals, pear, beans, soybeans, oilseed rape, tomato, bell pepper, melons, cabbage, potatoes and apples.

Examples of trees include: Abies sp., Eucalyptus sp., Picea sp., Pinus sp., Aesculus sp., Platanus sp., Tilia sp., Acer sp., Tsuga sp., Fraxinus sp., Sorbus sp., Betula sp., Crataegus sp., Ulmus sp., Quercus sp., Fagus sp., Salix sp., Populus sp.

Preferred trees include: from the tree species Aesculus: A. hippocastanum, A. pariflora, A. carnea; from the tree species Platanus: P. aceriflora, P. occidentalis, P. racemosa; from the tree species Picea: P. abies; from the tree species Pinus: P. radiate, P. ponderosa, P. contorta, P. sylvestre, P. elliottii, P. montecola, P. albicaulis, P. resinosa, P. palustris, P. taeda, P. flexilis, P. jeffregi, P. baksiana, P. strobes; from the tree species Eucalyptus: E. grandis, E. globulus, E. camadentis, E. nitens, E. obliqua, E. regnans, E. pilularus. Particularly preferred trees include: from the tree species Pinus: P. radiate, P. ponderosa, P. contorta, P. sylvestre, P. strobes; from the tree species Eucalyptus: E. grandis, E. globulus, E. camadentis. Very particularly preferred trees include: horse chestnut, Platanaceae, linden tree, maple tree.

The present invention can also be applied to any turfgrass types, including cool-season turfgrasses and warm-season turfgrasses. Examples of cool-season turfgrass types are bluegrasses (Poa spp.), such as Kentucky bluegrass (Poa pratensis L.), rough bluegrass (Poa trivialis L.), Canada bluegrass (Poa compressa L.), annual bluegrass (Poa annua L.), upland bluegrass (Poa glaucantha Gaudin), wood bluegrass (Poa nemoralis L.) and bulbous bluegrass (Poa bulbosa L.); bentgrasses (Agrostis spp.), such as creeping bentgrass (Agrostis palustris Huds.), colonial bentgrass (Agrostis tenuis Sibth.), velvet bentgrass (Agrostis canina L.), South German Mixed Bentgrass (Agrostis spp. including Agrostis tenius Sibth., Agrostis canina L., and Agrostis palustris Huds.), and redtop (Agrostis alba L.); fescues (Festucu spp.), such as red fescue (Festuca rubra L. spp. rubra), creeping fescue (Festuca rubra L.), chewings fescue (Festuca rubra commutata Gaud.), sheep fescue (Festuca ovina L.), hard fescue (Festuca longifolia Thuill.), hair fescue (Festucu capillata L.), tall fescue (Festuca arundinacea Schreb.) and meadow fescue (Festuca elanor L.); ryegrasses, (Lolium spp.), such as annual ryegrass (Lolium multiflorum Lam.), perennial ryegrass (Lolium perenne L.) and italian ryegrass (Lolium multiflorum Lam.); and wheatgrasses, (Agropyron spp.), such as fairway wheatgrass (Agropyron cristatum (L.) Gaertn.), crested wheatgrass (Agropyron desertorum (Fisch.) Schult.) and western wheatgrass (Agropyron smithii Rydb.). Examples of further cool-season turfgrasses are beachgrass (Ammophila breviligulata Fern.), smooth bromegrass (Bromus inermis Leyss.), cattails such as Timothy (Phleum pratense L.), sand cattail (Phleum subulatum L.), orchard grass (Dactylis glomerata L.), weeping alkaligrass (Puccinellia distans (L.) Parl.) and crested dog's-tail (Cynosurus cristatus L.). Examples of warm-season turfgrasses are Bermuda grass (Cynodon spp. L. C. Rich), zoysia grass (Zoysia spp. Willd.), St. Augustine grass (Stenotaphrum secundatum Walt Kuntze), centipede grass (Eremochloa ophiuroides Munro Hack.), carpet grass (Axonopus affinis Chase), Bahia grass (Paspalum notatum Flugge), Kikuyu grass (Pennisetum clandestinum Hochst. ex Chiov.), buffalo grass (Buchloe dactyloids (Nutt.) Engelm.), Blue grama (Bouteloua gracilis (H.B.K.) Lag. ex Griffiths), seashore paspalum (Paspalum vaginatum Swartz) and sideoats grama (Bouteloua curtipendula (Michx. Torr.). Cool-season turfgrasses are generally preferred for the inventive use. Particular preference is given to bluegrass, bentgrass and redtop, fescues and ryegrasses. Bentgrass is especially preferred.

Depending on the plant species or plant cultivars, and the location and growth conditions (soils, climate, vegetation period, diet) thereof, the inventive treatment may also result in superadditive (“synergistic”) effects. For example, the following effects extending beyond the effects that are actually to be expected are possible: reduced application rates and/or broadening of the activity spectrum and/or an increase in the activity of the compounds and compositions usable in accordance with the invention, better plant growth, increased tolerance to high or low temperatures, increased tolerance to drought or to levels of water or soil salinity, enhanced flowering performance, easier harvesting, accelerated ripening, higher yields, higher quality and/or higher nutritional value of the harvested products, increased storage life and/or processibility of the harvested products.

The transgenic plants or plant cultivars (those obtained by genetic engineering) which are to be treated with preference in accordance with the invention include all plants which, through the genetic modification, received genetic material which imparts particular advantageous useful properties (“traits”) to these plants. Examples of such properties are better plant growth, increased tolerance to high or low temperatures, increased tolerance to drought or to levels of water or soil salinity, enhanced flowering performance, easier harvesting, accelerated ripening, higher yields, higher quality and/or higher nutritional value of the harvested products, better storage life and/or processibility of the harvested products. Further and particularly emphasized examples of such properties are an improved defence of the plants against animal and microbial pests, such as against insects, mites, phytopathogenic fungi, bacteria and/or viruses, and also increased tolerance of the plants to certain herbicidally active ingredients. Examples of transgenic plants include the important crop plants, such as cereals (wheat, rice), maize, soybeans, potatoes, sugarbeet, tomatoes, peas and other vegetable types, cotton, tobacco, oilseed rape, and also fruit plants (with the following fruits: apples, pears, citrus fruits and grapes), particular emphasis being given to maize, soybeans, potatoes, cotton, tobacco and oilseed rape. Traits that are particularly emphasized are improved defence of the plants against insects, arachnids, nematodes, slugs and snails by toxins formed in the plants, especially those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF, and also combinations thereof) (referred to hereinafter as “Bt plants”). Traits that are also particularly emphasized are the improved defence of plants against fungi, bacteria and viruses by systemic acquired resistance (SAR), systemin, phytoalexins, elicitors and also resistance genes and correspondingly expressed proteins and toxins. Traits that are additionally particularly emphasized are the increased tolerance of the plants to certain active herbicidal compounds, for example imidazolinones, sulphonylureas, glyphosate or phosphinothricin (for example the “PAT” gene). The genes which impart the desired traits in question may also be present in combinations with one another in the transgenic plants. Examples of Bt plants include maize varieties, cotton varieties, soya bean varieties and potato varieties that are sold under the commercial names YIELD GARD® (e.g. maize, cotton, soya beans), KnockOut® (e.g. maize), StarLink® (e.g. maize), Bollgard® (cotton), Nucotn® (cotton) and NewLeaf® (potatoes). Examples of herbicide-tolerant plants include maize varieties, cotton varieties and soya bean varieties which are sold under the commercial designations Roundup Ready® (tolerance to glyphosate e g maize, cotton, soya beans), Liberty Link® (tolerance to phosphinothricin, e.g. oilseed rape), IMI® (tolerance to imidazolinones) and STS® (tolerance to sulphonylureas, e.g. maize) Herbicide-resistant plants (bred conventionally for herbicide tolerance) also include the varieties sold under the designation Clearfield® (e.g. maize). Of course, these statements also apply to plant cultivars which have these genetic traits or genetic traits which are still to be developed and will be developed and/or marketed in the future.

The plants listed can be treated in accordance with the invention in a particularly advantageous manner with the compounds of the general formula (I) and/or the inventive active ingredient mixtures. The areas of preference stated above for the active ingredients or mixtures also apply to the treatment of these plants. Particular emphasis is given to the treatment of plants with the compounds or mixtures specifically mentioned in the present text.

In the animal health field, i.e. in the field of veterinary medicine, the compounds of the formula (I) are active against animal parasites, in particular ectoparasites or endoparasites. The term “endoparasites” includes particularly helminths and protozoans, such as coccidia. Ectoparasites are typically and preferably arthropods, particularly insects and acarids.

In the field of veterinary medicine, the compounds of the formula (I) having favourable homeotherm toxicity are suitable for controlling parasites which occur in animal breeding and animal husbandry in livestock, breeding animals, zoo animals, laboratory animals, experimental animals and domestic animals. They are active against all or specific stages of development of the parasites.

Agricultural livestock include, for example, mammals such as sheep, goats, horses, donkeys, camels, buffalo, rabbits, reindeer, fallow deer, and particularly cattle and pigs; poultry such as turkeys, ducks, geese, and particularly chickens; fish and crustaceans, for example in aquaculture, and also insects such as bees.

Domestic animals include, for example, mammals, such as hamsters, guinea pigs, rats, mice, chinchillas, ferrets, and particularly dogs, cats, cage birds, reptiles, amphibians and aquarium fish.

In a preferred embodiment, the compounds of the formula (I) are administered to mammals.

In another preferred embodiment, the compounds of the formula (I) are administered to birds, namely caged birds and particularly poultry.

Use of the compounds of the formula (I) for the control of animal parasites is intended to reduce or prevent illness, cases of deaths and reductions in performance (in the case of meat, milk, wool, hides, eggs, honey and the like), such that more economical and simpler animal keeping is enabled and better animal well-being is achievable.

In relation to the animal health field, the term “control” or “controlling” means that the compounds of the formula (I) are effective in reducing the incidence of the particular parasite in an animal infected with such parasites to an innocuous degree. More specifically, “controlling” in the present context means that the compound of the formula (I) can kill the respective parasite, inhibit its growth, or inhibit its proliferation.

Arthropods include:

from the order of the Anoplurida, for example Haematopinus spp., Linognathus spp., Pediculus spp., Phtirus spp., Solenopotes spp.; from the order of the Mallophagida and the suborders Amblycerina and Ischnocerina, for example Trimenopon spp., Menopon spp., Trinoton spp., Bovicola spp., Werneckiella spp., Lepikentron spp., Damalina spp., Trichodectes spp., Felicola spp.; from the order of the Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp., Rhinoestrus spp., Tipula spp.; from the order of the Siphonapterida, for example Pulex spp., Ctenocephalides spp., Tunga spp., Xenopsylla spp., Ceratophyllus spp.;

from the order of the Heteropterida, for example Cimex spp., Triatoma spp., Rhodnius spp., Panstrongylus spp.; and also nuisance and hygiene pests from the order of the Blattarida.

Arthropods further include:

from the subclass of the Acari (Acarina) and the order of the Metastigmata, for example from the family of Argasidae like Argas spp., Ornithodorus spp., Otobius spp., from the family of Ixodidae like Ixodes spp., Amblyomma spp., Rhipicephalus (Boophilus) spp Dermacentor spp., Haemophysalis spp., Hyalomma spp., Rhipicephalus spp. (the original genus of multi-host ticks); from the order of Mesostigmata like Dermanyssus spp., Ornithonyssus spp., Pneumonyssus spp., Raillietia spp., Pneumonyssus spp., Sternostoma spp., Varroa spp., Acarapis spp.; from the order of the Actinedida (Prostigmata), for example Acarapis spp., Cheyletiella spp., Ornithocheyletia spp., Myobia spp., Psorergates spp., Demodex spp., Trombicula spp., Neotrombiculla spp., Listrophorus spp.; and from the order of the Acaridida (Astigmata), for example Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

Parasitic Protozoa include:

Mastigophora (Flagellata), for example Trypanosomatidae, for example, Trypanosoma b. brucei, T.b. gambiense, T.b. rhodesiense, T. congolense, T. cruzi, T. evansi, T. equinum, T. lewisi, T. percae, T. simiae, T. vivax, Leishmania brasiliensis, L. donovani, L. tropica, for example Trichomonadidae, for example, Giardia lamblia, G. canis;

Sarcomastigophora (Rhizopoda) such as Entamoebidae, for example, Entamoeba histolytica, Hartmanellidae, for example, Acanthamoeba sp., Harmanella sp.;

Apicomplexa (Sporozoa) such as Eimeridae, for example, Eimeria acervulina, E. adenoides, E. alabamensis, E. anatis, E. anserina, E. arloingi, E. ashata, E. auburnensis, E. bovis, E. brunetti, E. canis, E. chinchillae, E. clupearum, E. columbae, E. contorta, E. crandalis, E. debliecki, E. dispersa, E. ellipsoidales, E. falciformis, E. faurei, E. flavescens, E. gallopavonis, E. hagani, E. intestinalis, E. iroquoina, E. irresidua, E. labbeana, E. leucarti, E. magna, E. maxima, E. media, E. meleagridis, E. meleagrimitis, E. mitis, E. necatrix, E. ninakohlyakimovae, E. ovis, E. parva, E. pavonis, E. perforans, E. phasani, E. piriformis, E. praecox, E. residua, E. scabra, E. spec., E. stiedai, E. suis, E. tenella, E. truncata, E. truttae, E. zuernii, Globidium spec., Isospora belli, I. canis, I. felis, I. ohioensis, I. rivolta, I. spec., I. suis, Cystisospora spec., Cryptosporidium spec., in particular C. parvum; such as Toxoplasmadidae, for example, Toxoplasma gondii, Hammondia heydornii, Neospora caninum, Besnoitia besnoitii; such as Sarcocystidae, for example, Sarcocystis bovicanis, S. bovihominis, S. ovicanis, S. ovifelis, S. neurona, S. spec., S. suihominis, such as Leucozoidae, for example, Leucozytozoon simondi, such as Plasmodiidae, for example, Plasmodium berghei, P. falciparum, P. malariae, P. ovale, P. vivax, P. spec., such as Piroplasmea, for example, Babesia argentina, B. bovis, B. canis, B. spec., Theileria parva, Theileria spec., such as Adeleina, for example, Hepatozoon canis, H. spec.

Pathogenic endoparasites, which are helminths, include Platyhelmintha (e.g. Monogenea, cestodes and trematodes), nematodes, Acanthocephala, and Pentastoma. These include:

Monogenea: e.g.: Gyrodactylus spp., Dactylogyrus spp., Polystoma spp.

Cestodes: from the order of the Pseudophyllidea for example: Diphyllobothrium spp., Spirometra spp., Schistocephalus spp., Ligula spp., Bothridium spp., Diplogonoporus spp.;

from the order of the Cyclophyllida, for example: Mesocestoides spp., Anoplocephala spp., Paranoplocephala spp., Moniezia spp., Thysanosoma spp., Thysaniezia spp., Avitellina spp., Stilesia spp., Cittotaenia spp., Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.;

Trematodes: from the class of the Digenea, for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fasciolides spp., Fasciolopsis spp., Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp., Metorchis spp., Heterophyes spp., Metagonimus spp.;

Nematodes: Trichinellida, for example: Trichuris spp., Capillaria spp., Paracapillaria spp., Eucoleus spp., Trichomosoides spp., Trichinella spp.;

from the order of the Tylenchida, for example: Micronema spp., Strongyloides spp.;

from the order of the Rhabditida, for example: Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp., Uncinaria spp., Necator spp., Bunostomum spp., Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp. Parelaphostrongylus spp., Crenosoma spp., Paracrenosoma spp., Oslerus spp., Angiostrongylus spp., Aelurostrongylus spp., Filaroides spp., Parafilaroides spp., Trichostrongylus spp., Haemonchus spp., Ostertagia spp., Teladorsagia spp., Marshallagia spp., Cooperia spp., Nippostrongylus spp., Heligmosomoides spp., Nematodirus spp., Hyostrongylus spp., Obeliscoides spp., Amidostomum spp., Ollulanus spp.;

from the order of the Spirurida, for example: Oxyuris spp., Enterobius spp., Passalurus spp., Syphacia spp., Aspiculuris spp., Heterakis spp.; Ascaris spp., Toxascaris spp., Toxocara spp., Baylisascaris spp., Parascaris spp., Anisakis spp., Ascaridia spp.; Gnathostoma spp., Physaloptera spp., Thelazia spp., Gongylonema spp., Habronema spp., Parabronema spp., Draschia spp., Dracunculus spp.; Stephanofilaria spp., Parafilaria spp., Setaria spp., Loa spp., Dirofilaria spp., Litomosoides spp., Brugia spp., Wuchereria spp., Onchocerca spp., Spirocerca spp.;

Acanthocephala: from the order of the Oligacanthorhynchida, for example: Macracanthorhynchus spp., Prosthenorchis spp.; from the order of the Polymorphida, for example: Filicollis spp.; from the order of the Moniliformida, for example: Moniliformis spp.;

from the order of the Echinorhynchida, for example, Acanthocephalus spp., Echinorhynchus spp., Leptorhynchoides spp.;

Pentastoma: from the order of the Porocephalida, for example, Linguatula spp.

In the veterinary field and in animal keeping, the compounds of the formula (I) are administered by methods generally known in the art, such as via the enteral, parenteral, dermal or nasal route in the form of suitable preparations. Administration can be effected prophylactically or therapeutically.

Thus, one embodiment of the present invention refers to the use of a compound of the formula (I) as medicament.

A further aspect refers to the use of a compound of the formula (I) as an antiendoparasitic agent, in particular a helminthicidal agent or antiprotozoic agent. Compounds of the formula (I) are suitable for use as an antiendoparasitic agent, especially as a helminthicidal agent or antiprotozoic agent, for example in animal husbandry, in animal breeding, in animal housing and in the hygiene sector.

A further aspect in turn relates to the use of a compound of the formula (I) as an antiectoparasitic agent, especially an arthropodicide such as an insecticide or an acaricide. A further aspect relates to the use of a compound of the formula (I) as an antiectoparasitic agent, especially an arthropodicide such as an insecticide or an acaricide, for example in animal husbandry, in animal breeding, in stables or in the hygiene sector.

The compounds of the formula (I) can also be used in vector control. In the context of the present invention, a vector is an arthropod, especially an insect or arachnid, capable of transmitting pathogens, for example, viruses, worms, single-cell organisms and bacteria, from a reservoir (plant, animal, human, etc.) to a host. The pathogens can be transmitted either mechanically (for example trachoma by non-stinging flies) to a host or by injection (for example malaria parasites by mosquitoes) into a host.

Examples of vectors and the diseases or pathogens they transmit are:

### 1) Mosquitoes

Anopheles: malaria, filariasis;
Culex: Japanese encephalitis, filariasis, other viral diseases, transmission of worms;
Aedes: yellow fever, dengue fever, filariasis, other viral diseases;
Simuliidae: transmission of worms, in particular Onchocerca volvulus;
   2) Lice: skin infections, epidemic typhus;
   3) Fleas: plague, endemic typhus;
   4) Flies: sleeping sickness (trypanosomiasis); cholera, other bacterial diseases;
   5) Mites: acariosis, epidemic typhus, rickettsialpox, tularaemia, Saint Louis encephalitis, tick-borne encephalitis (TBE), Crimean-Congo haemorrhagic fever, borreliosis;

6) Ticks: borellioses such as Borrelia duttoni, tick-borne encephalitis, Q fever (Coxiella burnetii), babesioses (Babesia canis canis).

Examples of vectors in the context of the present invention are insects, for example aphids, flies, leafhoppers or thrips, which can transmit plant viruses to plants. Other vectors capable of transmitting plant viruses are spider mites, lice, beetles and nematodes.

Further examples of vectors in the context of the present invention are insects and arachnids such as mosquitoes, especially of the genera Aedes, Anopheles, for example A. gambiae, A. arabiensis, A. funestus, A. dirus (malaria) and Culex, lice, fleas, flies, mites and ticks, which can transmit pathogens to animals and/or humans.

Vector control is also possible if the compounds of the formula (I) are resistance-breaking.

Compounds of the formula (I) are suitable for use in the prevention of diseases and/or pathogens transmitted by vectors. Thus, a further aspect of the present invention is the use of compounds of the formula (I) for vector control, for example in agriculture, in horticulture, in forestry, in gardens and in leisure facilities, and also in the protection of materials and stored products.

The active ingredients of the formula (I) and compositions thereof are suitable for controlling animal pests in the hygiene sector. In particular, the invention can be used in the domestic sector, in the hygiene sector and in the protection of stored products, particularly for control of insects, arachnids and mites encountered in enclosed spaces, for example dwellings, factory halls, offices, vehicle cabins. For control of animal pests, the active ingredients or compositions are used alone or in combination with other active ingredients and/or auxiliaries. They are preferably used in domestic insecticide products. The inventive active ingredients are effective against sensitive and resistant species, and against all developmental stages.

These pests include, for example, pests from the class Arachnida, from the orders Scorpiones, Araneae and Opiliones, from the classes Chilopoda and Diplopoda, from the class Insecta the order Blattodea, from the orders Coleoptera, Dermaptera, Diptera, Heteroptera, Hymenoptera, Isoptera, Lepidoptera, Phthiraptera, Psocoptera, Saltatoria or Orthoptera, Siphonaptera and Zygentoma and from the class Malacostraca the order Isopoda.

Application is effected, for example, in aerosols, unpressurized spray products, for example pump and atomizer sprays, automatic fogging systems, foggers, foams, gels, evaporator products with evaporator tablets made of cellulose or plastic, liquid evaporators, gel and membrane evaporators, propeller-driven evaporators, energy-free, or passive, evaporation systems, moth papers, moth bags and moth gels, as granules or dusts, in baits for spreading or in bait stations.

The active ingredients of the formula (I) and compositions comprising them are suitable for protection of industrial materials against attack or destruction by insects, for example from the orders of Coleoptera, Hymenoptera, Isoptera, Lepidoptera, Psocoptera and Zygentoma.

Industrial materials in the present context are understood to mean inanimate materials, such as preferably plastics, adhesives, sizes, papers and cards, leather, wood, processed wood products and coating compositions. The employment of the invention for protection of wood is particularly preferred.

In one embodiment of the invention, the inventive compositions also comprise at least one further insecticide and/or at least one fungicide.

In a further embodiment, this inventive composition is a ready-to-use composition, meaning that it can be applied to the material in question without further modifications. Useful further insecticides or fungicides include those mentioned above.

It has also been found that, surprisingly, the inventive active ingredients and compositions can be used to protect objects which come into contact with saltwater or brackish water, especially hulls, screens, nets, buildings, moorings and signalling systems, against fouling. The inventive active ingredients and compositions can again be used alone or in combinations with other active ingredients as antifouling compositions.

The Preparation and Use Examples which follow illustrate the invention without limiting it.

### PREPARATION EXAMPLES

### Synthesis Example 1

### 6-Chloro-N-(1-{[3-chloro-5-(trifluoromethyl)pyridin-2-yl]methyl}-1H-pyrazol-3-yl)nicotinamide (Compound I-1-118 in Table 1)

### Stage 1: 3-Chloro-2-[(3-nitro-1H-pyrazol-1-yl)methyl]-5-(trifluoromethyl)pyridine (by Route A-1)

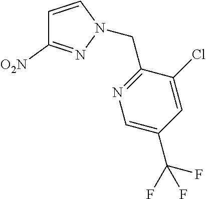 [SMILES: O=[N+]([O-])c1ccn(Cc2ncc(C(F)(F)F)cc2Cl)n1]

A solution of 3-nitro-1H-pyrazole (1.60 g) in 40 ml of acetonitrile was admixed with potassium carbonate (3.91 g) and 3-chloro-2-(chloromethyl)-5-(trifluoromethyl)pyridine (4.88 g), and the mixture was stirred at 60° C. for 12 h. Water was added, and the resulting precipitate was filtered off with suction and dried under reduced pressure. The crude product was purified by column chromatography on silica gel with cyclohexane/ethyl acetate as eluent. This gave 3.50 g of the title compound. HPLC-MS: log P=2.67; mass (m/z): 307.1 (M+H)⁺; 1H NMR (DMSO-D₆) 5.88 (s, 2H), 7.12-7.13 (m, 1H), 8.15-8.16 (m, 1H), 8.57 (s, 1H), 8.89 (s, 1H).

### Stage 2: 1-{[3-Chloro-5-(trifluoromethyl)pyridin-2-yl]methyl}-1H-pyrazole-3-amine

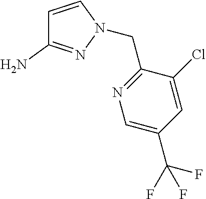 [SMILES: Nc1ccn(Cc2ncc(C(F)(F)F)cc2Cl)n1]

A solution of 3-chloro-2-[(3-nitro-1H-pyrazol-1-yl)methyl]-5-(trifluoromethyl)pyridine (3.40 g) in ethanol was admixed with SnCl₂ (8.30 g) and heated under reflux for 1 h. The reaction solution was concentrated under reduced pressure, and the residue was taken up with ice-water, brought to pH 9 with 10% NaOH solution and extracted with ethyl acetate. The organic phase was dried over magnesium sulphate, filtered and concentrated under reduced pressure. This gave 2.95 g of the title compound. HPLC-MS: log P=1.36; mass (m/z): 277.1 (M+H)⁺; 1H NMR (DMSO-D₆) 4.56 (br. s, 2H), 5.35 (s, 2H), 5.42 (s, 1H), 7.45 (s, 1H), 8.46 (s, 1H), 8.89 (s, 1H).

### Stage 3: 6-Chloro-N-(1-{[3-chloro-5-(trifluoromethyl)pyridin-2-yl]methyl}-1H-pyrazol-3-yl)nicotinamide

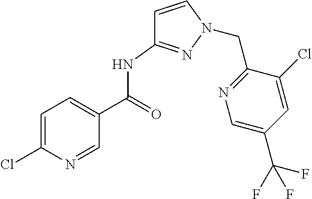 [SMILES: O=C(Nc1ccn(Cc2ncc(C(F)(F)F)cc2Cl)n1)c1ccc(Cl)nc1]

A solution of 6-chloronicotinic acid (56 mg) in 2 ml of 1,4-dioxane was admixed with 1-ethyl-3-(3-dimethylaminopropyl)carbodiimide hydrochloride (83 mg), and the mixture was stirred at room temperature for 30 minutes. Subsequently, 1-{[3-chloro-5-(trifluoromethyl)pyridin-2-yl]methyl}-1H-pyrazole-3-amine (100 mg) was added and the resulting reaction mixture was stirred at room temperature for 18 h. The mixture was concentrated under reduced pressure, the residue was admixed with water and extracted with ethyl acetate, and the organic phase was dried over magnesium sulphate, filtered and concentrated under reduced pressure. This gave 92 mg of the title compound. HPLC-MS: log P=2.64; mass (m/z): 416.1 (M)⁺; 1H NMR (DMSO-D₆) 5.63 (s, 2H), 6.69 (s, 1H), 7.63-7.65 (m, 1H), 7.86 (s, 1H), 8.31-8.34 (m, 1H), 8.52 (s, 1H), 8.92 (s, 2H), 11.18 (s, 1H).

### Synthesis Example 2

### Methyl 3-{[(5-fluoropyridin-3-yl)carbonyl]amino}-1-methyl-1H-pyrazole-5-carboxylate (Compound I-1-134 in Table 1)

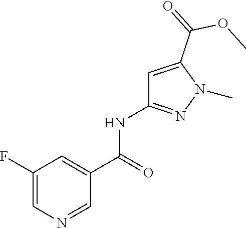 [SMILES: COC(=O)c1cc(NC(=O)c2cncc(F)c2)nn1C]

A solution of 5-fluoronicotinic acid (174 mg) in 10 ml of dichloromethane was admixed with 1-ethyl-3-(3-dimethylaminopropyl)carbodiimide hydrochloride (237 mg), 1-hydroxy-1H-benzotriazole (167 mg), diisopropylethylamine (0.26 ml) and 4-(dimethylamino)pyridine (56 mg), and the mixture was stirred at room temperature for 5 minutes. Subsequently, methyl 3-amino-1-methyl-1H-pyrazole-5-carboxylate (120 mg) was added and the resulting reaction mixture was stirred at room temperature for 48 h. The mixture was washed with 3 ml of water and the organic phase was dried over sodium sulphate, filtered and concentrated under reduced pressure. The crude product was purified by column chromatography on silica gel with cyclohexane/ethyl acetate (4:6) as eluent. This gave 175 mg of the title compound. HPLC-MS: log P=1.5; mass (m/z): 279.1 (M+H)⁺; 1H NMR (DMSO-D₆) 3.86 (s, 3H), 4.06 (s, 3H), 7.19 (s, 1H), 8.23-8.26 (m, 1H), 8.78-8.79 (m, 1H), 9.01 (s, 1H), 11.43 (s, 1H).

### Synthesis Example 3

### tert-Butyl 5-methyl-3-[(pyridin-3-ylcarbonyl)amino]-1H-pyrazole-1-carboxylate (Compound I-1-18 in Table 1)

### Stage 1: tert-Butyl 3-amino-5-methyl-1H-pyrazole-1-carboxylate (by Route B-1)

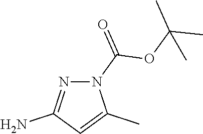

5-Methyl-1H-pyrazole-3-amine (1.00 g) was initially charged in dichloromethane (50 ml), triethylamine (1.58 ml) and di-tert-butyl dicarbonate (2.47 g) were added, and the resulting solution was stirred at room temperature for 3 h. The reaction mixture was subsequently washed with sat. NaCl solution, and the organic phase was dried over sodium sulphate, filtered and concentrated under reduced pressure. The crude product was purified by column chromatography on silica gel with cyclohexane/ethyl acetate (3:7) as eluent. This gave 1.48 g of the title compound. HPLC-MS: log P=1.23; 1H NMR (MeOD) 1.61 (s, 9H), 2.10 (s, 3H), 5.24 (s, 1H).

### Stage 2: tert-Butyl 5-methyl-3-[(pyridin-3-ylcarbonyl)amino]-1H-pyrazole-1-carboxylate

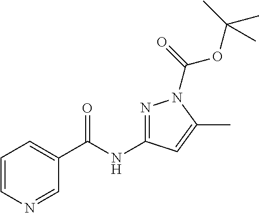 [SMILES: Cc1cc(NC(=O)c2cccnc2)nn1C(=O)OC(C)(C)C]

A solution of tert-butyl 3-amino-5-methyl-1H-pyrazole-1-carboxylate (11.1 g) in dichloromethane (740 ml) was admixed with triethylamine (15.69 ml) and nicotinoyl chloride hydrochloride (10.018 g), and stirred at room temperature for 48 h. The reaction mixture was subsequently washed with 200 ml of sat. NaCl solution, and the organic phase was dried over sodium sulphate, filtered and concentrated under reduced pressure. The crude product was purified by column chromatography on silica gel with dichloromethane/ethyl acetate (7:3) as eluent. This gave 3.98 g of the title compound. HPLC-MS: log P=2.04; mass (m/z): 303.1 (M+H)⁺; 1H NMR (DMSO-D₆) 1.55 (s, 9H), 2.21 (s, 3H), 6.60 (s, 1H), 7.61-7.65 (m, 1H), 8.24-8.27 (m, 1H), 8.81-8.83 (m, 1H), 9.08 (s, 1H), 10.80 (s, 1H).

### Synthesis Example 4

### N-(5-Methyl-1H-pyrazol-3-yl)nicotinamide (Compound I-1-17 in Table 1)

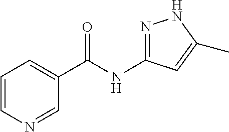

A solution of tert-butyl 5-methyl-3-[(pyridin-3-ylcarbonyl)amino]-1H-pyrazole-1-carboxylate (300 mg) in dichloromethane (10 ml) was admixed with trifluoroacetic acid (2.29 ml) and stirred at room temperature for 2 h. The reaction mixture was subsequently concentrated under reduced pressure and gave 200 mg of the title compound. HPLC-MS: log P=0.3; mass (m/z): 203.1 (M+H)⁺; 1H NMR (DMSO-D₆) 2.27 (s, 3H), 6.44 (s, 1H), 7.94-7.98 (m, 1H), 8.80-8.82 (m, 1H), 8.95-8.97 (m, 1H), 9.33 (s, 1H), 11.52 (s, 1H).

### Synthesis Example 5

### N-[5-(Difluoromethoxy)-1-methyl-1H-pyrazol-3-yl]-5-fluoronicotinamide (Compound I-1-112 in Table 1)

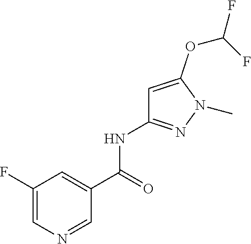 [SMILES: Cn1nc(NC(=O)c2cncc(F)c2)cc1OC(F)F]

A solution of 5-fluoronicotinic acid (141 mg) in 2 ml of tetrahydrofuran was admixed with 1-ethyl-3-(3-dimethylaminopropyl)carbodiimide hydrochloride (230 mg), and the mixture was stirred at room temperature for 30 minutes. Subsequently, a solution of 5-(difluoromethoxy)-1-methyl-1H-pyrazole-3-amine (163 mg) in 0.5 ml of tetrahydrofuran was added, and the resulting reaction mixture was stirred at room temperature for 12 h. The mixture was concentrated under reduced pressure and the crude product was purified by column chromatography on silica gel with dichloromethane/isopropanol as eluent. This gave 180 mg of the title compound. HPLC-MS: log P=1.56; mass (m/z): 287.0 (M+H)⁺; 1H NMR (DMSO-D₆) 3.63 (s, 3H), 6.45 (s, 1H), 7.33 (t, 1H), 8.20-8.24 (m, 1H), 8.76-8.77 (m, 1H), 8.98-8.99 (m, 1H), 11.23 (s, 1H).

### Synthesis Example 6

### N-[1-(2-Chlorobenzoyl)-5-methyl-1H-pyrazol-3-yl]nicotinamide (Compound I-1-122 in Table 1)

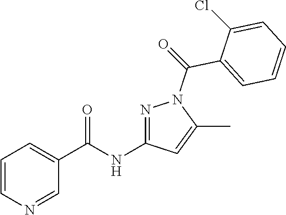 [SMILES: Cc1cc(NC(=O)c2cccnc2)nn1C(=O)c1ccccc1Cl]

A solution of N-(5-methyl-1H-pyrazol-3-yl)nicotinamide (0.1 g) in dichloromethane (5 ml) was admixed with triethylamine (0.07 ml) and 2-chlorobenzoyl chloride (0.087 g) and stirred at room temperature for 48 h. The reaction mixture was subsequently washed with water, and the organic phase was dried over sodium sulphate, filtered and concentrated under reduced pressure. The crude product was purified by column chromatography on silica gel with cyclohexane/ethyl acetate as eluent. This gave 11 mg of the title compound. HPLC-MS: log P=2.27; mass (m/z): 341.0 (M+H)⁺; 1H NMR (DMSO-D₆) 2.68 (s, 3H), 7.01 (s, 1H), 7.45-7.59 (m, 4H), 7.64-7.66 (m, 1H), 8.26-8.29 (m, 1H), 8.70-8.72 (m, 1H), 9.05 (s, 1H), 11.41 (s, 1H). 13C NMR (DMF-D₇) 15.2, 107.0, 124.9, 128.5, 130.9, 131.0, 132.0, 133.2, 136.9, 137.1, 145.9, 150.8, 152.3, 154.2, 166.0, 168.4.

### Synthesis Example 7

### N-(1-Benzyl-5-methyl-1H-pyrazol-3-yl)pyridazine-4-carboxamide (Compound I-1-79 in Table 1)

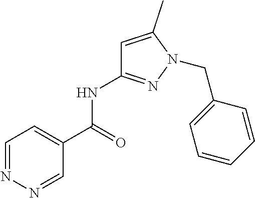 [SMILES: Cc1cc(NC(=O)c2ccnnc2)nn1Cc1ccccc1]

A solution of pyridazine-4-carboxylic acid (63 mg) in 2 ml of 1,4-dioxane was admixed with 1-ethyl-3-(3-dimethylaminopropyl)carbodiimide hydrochloride (117 mg), and the mixture was stirred at room temperature for 60 minutes. Subsequently, a solution of 1-benzyl-5-methyl-1H-pyrazole-3-amine (100 mg) in 0.5 ml of 1,4-dioxane was added and the resulting reaction mixture was stirred at room temperature for 24 h and at 50° C. for 12 h. The mixture was concentrated under reduced pressure, the residue was admixed with water and extracted with ethyl acetate, and the organic phase was dried over sodium sulphate, filtered and concentrated under reduced pressure. The residue was chromatographed on a preparative HPLC with water/acetonitrile as eluent. This gave 63 mg of the title compound. HPLC-MS: log P=1.67; mass (m/z): 294.1 (M+H)⁺; 1H NMR (DMSO-D₆) 2.26 (s, 3H), 5.23 (s, 2H), 6.52 (s, 1H), 7.14-7.36 (m, 5H), 8.08-8.10 (m, 1H), 9.39-9.41 (m, 1H), 9.61 (s, 1H), 11.17 (s, 1H).

### Synthesis Example 8

### N-[1-(Cyclopropylcarbonyl)-5-methyl-1H-pyrazol-3-yl]nicotinamide (Compound I-1-121 in Table 1)

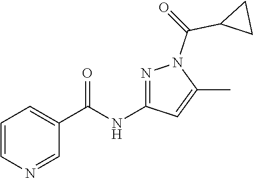 [SMILES: Cc1cc(NC(=O)c2cccnc2)nn1C(=O)C1CC1]

A solution of N-(5-methyl-1H-pyrazol-3-yl)nicotinamide (0.1 g) in dichloromethane (5 ml) was admixed with triethylamine (0.07 ml) and cyclopropanecarbonyl chloride (0.052 g) and stirred at room temperature for 48 h. The reaction mixture was subsequently washed with water, and the organic phase was dried over sodium sulphate, filtered and concentrated under reduced pressure. The crude product was purified by column chromatography on silica gel with cyclohexane/ethyl acetate as eluent. This gave 21 mg of the title compound. HPLC-MS: log P=1.65; mass (m/z): 271.1 (M+H)⁺; 1H NMR (DMF-D₇) 1.09-1.17 (m, 4H), 2.58 (s, 3H), 3.06-3.10 (m, 1H), 6.96 (s, 1H), 7.59-7.62 (m, 1H), 8.50-8.52 (m, 1H), 8.81-8.82 (m, 1H), 9.32 (s, 1H), 11.47 (s, 1H).

### Synthesis Example 9

### N-[5-Bromo-1-(pyrimidin-2-ylmethyl)-1H-pyrazol-3-yl]nicotinamide (Compound I-1-140 in Table 1)

### Stage 1: 2-(5-Bromo-1H-pyrazol-3-yl)-1H-isoindole-1,3(2H)-dione (by Route D-1)

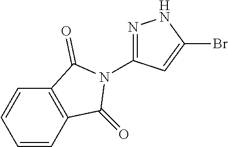 [SMILES: O=C1c2ccccc2C(=O)N1c1cc(Br)[nH]n1]

A solution of 5-bromo-1H-pyrazole-3-amine (1.00 g) in 50 ml of 1,4-dioxane was admixed with 2 g of molecular sieve (2 Å) and 2-benzofuran-1,3-dione (1.28 g) and heated under reflux overnight. Subsequently, the reaction mixture was filtered and the filtrate was concentrated under reduced pressure. The crude product was stirred with tert-butyl methyl ether and the resulting solid was dried under reduced pressure. This gave 1.68 g of the title compound. HPLC-MS: log P=1.83; 1H NMR (DMSO-D₆) 6.56 (s, 1H), 7.94-8.01 (m, 4H), 13.62-13.88 (m, 1H).

### Stage 2: 2-[5-Bromo-1-(pyrimidin-2-ylmethyl)-1H-pyrazol-3-yl]-1H-isoindole-1,3(2H)-dione

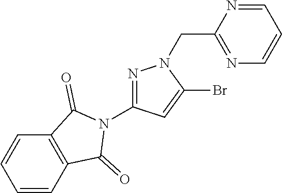 [SMILES: O=C1c2ccccc2C(=O)N1c1cc(Br)n(Cc2ncccn2)n1]

A solution of 2-(5-bromo-1H-pyrazol-3-yl)-1H-isoindole-1,3(2H)-dione (1.60 g) in 30 ml of acetonitrile was admixed with 2.27 g of potassium carbonate and 994 mg of 2-(chloromethyl)pyrimidine hydrochloride, and the mixture was heated under reflux for 3 h. The resulting reaction mixture was filtered and the filtrate was concentrated under reduced pressure. This left 2.00 g of the crude product, which was used without further purification for the next step. HPLC-MS: log P=2.02; mass (m/z): 385.0 (M+H)⁺.

### Stage 3: 5-Bromo-1-(pyrimidin-2-ylmethyl)-1H-pyrazole-3-amine

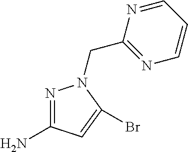 [SMILES: Nc1cc(Br)n(Cc2ncccn2)n1]

A mixture of 2-[5-bromo-1-(pyrimidin-2-ylmethyl)-1H-pyrazol-3-yl]-1H-isoindole-1,3(2H)-dione (2.00 g) and 4 ml of 2-aminoethanol was heated to 100° C. for 2 h. The resulting reaction mixture was concentrated under reduced pressure, the residue was taken up with ethyl acetate, 10 ml of 10% sodium hydroxide solution were added and the mixture was stirred at room temperature for 1 h. The organic phase was dried over sodium sulphate, filtered and concentrated under reduced pressure. The crude product was purified by column chromatography on silica gel with ethyl acetate/ethanol (9:1) as eluent. This gave 810 mg of 3-bromo-1-(pyrimidin-2-ylmethyl)-1H-pyrazole-5-amine and 110 mg of the title compound. HPLC-MS: log P=0.56; mass (m/z): 255.0 (M+H)⁺; 1H NMR (DMSO-D₆) 4.79 (br. s, 2H), 5.26 (s, 2H), 5.61 (s, 1H), 7.41-7.43 (m, 1H), 8.75-8.77 (m, 2H).

### Stage 4: N-[5-Bromo-1-(pyrimidin-2-ylmethyl)-1H-pyrazol-3-yl]nicotinamide

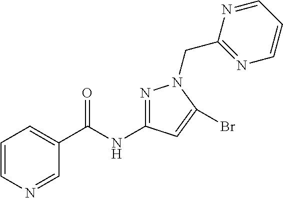 [SMILES: O=C(Nc1cc(Br)n(Cc2ncccn2)n1)c1cccnc1]

A solution of nicotinic acid (83 mg) in 10 ml of dichloromethane was admixed with 1-ethyl-3-(3-dimethylaminopropyl)carbodiimide hydrochloride (130 mg), 1-hydroxy-1H-benzotriazole (91 mg), diisopropylethylamine (0.15 ml) and 4-(dimethylamino)pyridine (31 mg), and the mixture was stirred at room temperature for 5 minutes. Subsequently, 5-bromo-1-(pyrimidin-2-ylmethyl)-1H-pyrazole-3-amine (108 mg) was added and the resulting reaction mixture was stirred at room temperature for 48 h. 3 ml of water were added, and the organic phase was dried over sodium sulphate, filtered and concentrated under reduced pressure. The crude product was purified by column chromatography on silica gel with ethyl acetate/ethanol (4:1) as eluent. This gave 99 mg of the title compound. HPLC-MS: log P=1.1; mass (m/z): 360.9 (M+H)⁺; 1H NMR (DMSO-D₆) 5.54 (s, 2H), 6.85 (s, 1H), 7.45-7.47 (m, 1H), 7.50-7.53 (m, 1H), 8.30-8.33 (m, 1H), 8.72-8.73 (m, 1H), 8.79-8.81 (m, 2H), 9.10-9.11 (m, 1H), 11.26 (s, 1H).

### Synthesis Example 10

### N-(1-Benzyl-5-ethyl-1H-pyrazol-3-yl)nicotinamide (Compound I-1-35 in Table 1)

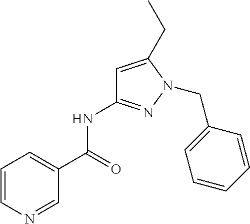 [SMILES: CCc1cc(NC(=O)c2cccnc2)nn1Cc1ccccc1]

A solution of nicotinic acid (61 mg) in 2 ml of 1,4-dioxane was admixed with 1-ethyl-3-(3-dimethylaminopropyl)carbodiimide hydrochloride (114 mg), and the mixture was stirred at room temperature for 30 minutes. Subsequently, a solution of 1-benzyl-5-ethyl-1H-pyrazole-3-amine (100 mg) in 0.5 ml of 1,4-dioxane was added and the resulting reaction mixture was stirred at room temperature for 24 h. The mixture was concentrated under reduced pressure, the residue was admixed with water and extracted with ethyl acetate, and the organic phase was dried over sodium sulphate, filtered and concentrated under reduced pressure. The crude product was purified by column chromatography on silica gel with cyclohexane/ethyl acetate as eluent. This gave 86 mg of the title compound. HPLC-MS: log P=2.03; mass (m/z): 307.1 (M+H)⁺; 1H NMR (DMSO-D₆) 1.17 (t, 3H), 2.62 (q, 2H), 5.23 (s, 2H), 6.54 (s, 1H), 7.13-7.35 (m, 5H), 7.46-7.50 (m, 1H), 8.29-8.32 (m, 1H), 8.69-8.70 (m, 1H), 9.11 (s, 1H), 10.81 (s, 1H).

### Synthesis Example 11

### N-[4-Methyl-1-(pyrimidin-2-ylmethyl)-1H-pyrazol-3-yl]nicotinamide (Compound I-1-136 in Table 1)

### Stage 1: 2-(4-Methyl-1H-pyrazol-3-yl)-1H-isoindole-1,3(2H)-dione (by Route D-1)

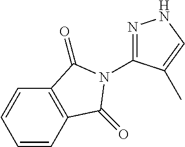 [SMILES: Cc1c[nH]nc1N1C(=O)c2ccccc2C1=O]

A solution of 4-methyl-1H-pyrazole-3-amine (1.00 g) in 50 ml of 1,4-dioxane was admixed with 2 g of molecular sieve (2 Å) and 2-benzofuran-1,3-dione (2.13 g) and heated under reflux overnight. Subsequently, the reaction mixture was filtered and the filtrate was concentrated under reduced pressure. The crude product was stirred with tert-butyl methyl ether and the resulting solid was dried under reduced pressure. This gave 2.30 g of the title compound. HPLC-MS: log P=1.34; mass (m/z): 228.1 (M+H)⁺; 1H NMR (DMSO-D₆) 1.87 (s, 3H), 7.68 (s, 1H), 7.92-8.02 (m, 4H), 12.89 (s, 1H).

### Stage 2: 2-[4-Methyl-1-(pyrimidin-2-ylmethyl)-1H-pyrazol-3-yl]-1H-isoindole-1,3(2H)-dione

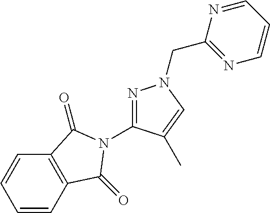 [SMILES: Cc1cn(Cc2ncccn2)nc1N1C(=O)c2ccccc2C1=O]

A solution of 2-(4-methyl-1H-pyrazol-3-yl)-1H-isoindole-1,3(2H)-dione (2.30 g) in 50 ml of acetonitrile was admixed with 4.19 g of potassium carbonate and 1.83 g of 2-(chloromethyl)pyrimidine hydrochloride, and the mixture was heated under reflux for 5 h. Another 1.39 g of potassium carbonate and 1.66 g of 2-(chloromethyl)pyrimidine hydrochloride were added and the mixture was heated under reflux for a further 3 h. The resulting reaction mixture was filtered and the filtrate was concentrated under reduced pressure. This left 3.50 g of the crude product, which was used without further purification for the next step. HPLC-MS: log P=1.58; mass (m/z): 320.1 (M+H)⁺.

### Stage 3: 4-Methyl-1-(pyrimidin-2-ylmethyl)-1H-pyrazole-3-amine

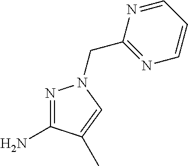

A mixture of 2-[4-methyl-1-(pyrimidin-2-ylmethyl)-1H-pyrazol-3-yl]-1H-isoindole-1,3(2H)-dione (3.50 g) and 6 ml of 2-aminoethanol was heated to 100° C. for 2 h. The resulting reaction mixture was concentrated under reduced pressure, the residue was taken up with ethyl acetate, 15 ml of 10% sodium hydroxide solution were added and the mixture was stirred at room temperature for 1 h. The organic phase was dried over sodium sulphate, filtered and concentrated under reduced pressure. The crude product was purified by column chromatography on silica gel with ethyl acetate/ethanol (4:1) as eluent. This gave 220 mg of 4-methyl-1-(pyrimidin-2-ylmethyl)-1H-pyrazole-5-amine and 250 mg of the title compound. HPLC-MS: log P=−0.22; mass (m/z): 190.1 (M+H)⁺; 1H NMR (DMSO-D₆) 1.81 (s, 3H), 4.32 (br. s, 2H), 5.14 (s, 2H), 7.24 (s, 1H), 7.38-7.41 (m, 1H), 8.74-8.75 (m, 2H).

### Stage 4: N-[4-Methyl-1-(pyrimidin-2-ylmethyl)-1H-pyrazol-3-yl]nicotinamide

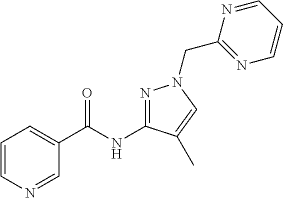 [SMILES: Cc1cn(Cc2ncccn2)nc1NC(=O)c1cccnc1]

A solution of nicotinic acid (145 mg) in 12 ml of dichloromethane was admixed with 1-ethyl-3-(3-dimethylaminopropyl)carbodiimide hydrochloride (226 mg), 1-hydroxy-1H-benzotriazole (159 mg), diisopropylethylamine (0.25 ml) and 4-(dimethylamino)pyridine (54 mg), and the mixture was stirred at room temperature for 5 minutes. Subsequently, 4-methyl-1-(pyrimidin-2-ylmethyl)-1H-pyrazole-3-amine (140 mg) was added and the resulting reaction mixture was stirred at room temperature for 18 h. The mixture was diluted with 10 ml of dichloromethane, 10 ml of water were added, and the organic phase was dried over sodium sulphate, filtered and concentrated under reduced pressure. The crude product was purified by column chromatography on silica gel with ethyl acetate/ethanol (4:1) as eluent. This gave 93 mg of the title compound. HPLC-MS: log P=0.44; mass (m/z): 295.1 (M+H)⁺; 1H NMR (DMSO-D₆) 1.91 (s, 3H), 5.43 (s, 2H), 7.43-7.46 (m, 1H), 7.51-7.54 (m, 1H), 7.66 (s, 1H), 8.28-8.31 (m, 1H), 8.72-8.74 (m, 1H), 8.79-8.80 (m, 2H), 9.10 (s, 1H), 10.39 (s, 1H).

### Synthesis Example 12

### 5-Fluoro-N-[5-methyl-1-(pyrimidin-2-ylmethyl)-1H-pyrazol-3-yl]nicotinamide (Compound I-1-7 in Table 1)

### Stage 1: Methyl 5-methyl-1-(pyrimidin-2-ylmethyl)-1H-pyrazole-3-carboxylate (by Route C-1)

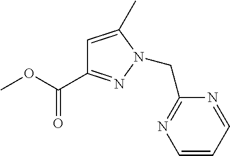 [SMILES: COC(=O)c1cc(C)n(Cc2ncccn2)n1]

A solution of methyl 5-methyl-1H-pyrazole-3-carboxylate (4.60 g) in 90 ml of N,N-dimethylformamide was admixed with potassium carbonate (10.88 g), and the mixture was stirred at room temperature for 15 minutes. Subsequently, 2-(chloromethyl)pyrimidine hydrochloride (6.49 g) was added, and the reaction mixture was heated under reflux for 18 h, poured onto 300 ml of water and extracted with ethyl acetate. The organic phase was dried over sodium sulphate, filtered and concentrated under reduced pressure. The crude product was purified by column chromatography on silica gel with cyclohexane/ethyl acetate (1:9) as eluent. This gave 0.96 g of methyl 3-methyl-1-(pyrimidin-2-ylmethyl)-1H-pyrazole-5-carboxylate and 3.85 g of the title compound. HPLC-MS: log P=0.98; mass (m/z): 233.0 (M+H)⁺; 1H NMR (DMSO-D₆) 2.27 (s, 3H), 3.75 (s, 3H), 5.59. (s, 2H), 6.59 (s, 1H), 7.44-7.46 (m, 1H), 8.77-8.78 (m, 2H).

### Stage 2: 5-Methyl-1-(pyrimidin-2-ylmethyl)-1H-pyrazole-3-carboxylic acid

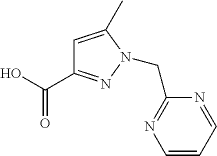 [SMILES: Cc1cc(C(=O)O)nn1Cc1ncccn1]

A solution of methyl 5-methyl-1-(pyrimidin-2-ylmethyl)-1H-pyrazole-3-carboxylate (3.80 g) in 70 ml of 1,4-dioxane was admixed dropwise with a solution of sodium hydroxide (0.85 g) in 15 ml of water, and the mixture was stirred at room temperature for 12 h. The resulting reaction mixture was concentrated under reduced pressure, the residue was taken up with 30 ml of water and acidified with 2 N hydrochloric acid, and the resulting solids were filtered off with suction and dried under reduced pressure. This gave 3.30 g of the title compound. HPLC-MS: log P=0.45; mass (m/z): 219.0 (M+H)+; 1H NMR (DMSO-D₆) 2.26 (s, 3H), 5.57 (s, 2H), 6.52 (s, 1H), 7.44-7.47 (m, 1H), 8.78-8.79 (m, 2H), 12.51 (s, 1H).

### Stage 3: tert-Butyl[5-methyl-1-(pyrimidin-2-ylmethyl)-1H-pyrazol-3-yl]carbamate

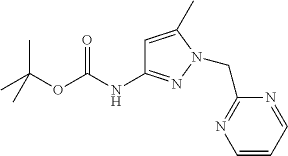 [SMILES: Cc1cc(NC(=O)OC(C)(C)C)nn1Cc1ncccn1]

A solution of 5-methyl-1-(pyrimidin-2-ylmethyl)-1H-pyrazole-3-carboxylic acid (0.50 g) in 2-methylpropan-2-ol (5.09 g) was admixed with triethylamine (0.696 g), heated to 80° C. and admixed dropwise with diphenyl phosphorazidate (0.694 g). The reaction mixture was stirred at 80° C. for 3 h and at room temperature for 12 h, then admixed with sat. NaCl solution and extracted twice with ethyl acetate. The combined organic phases were dried over sodium sulphate, filtered and concentrated under reduced pressure. The crude product was purified by column chromatography on silica gel with cyclohexane/ethyl acetate as eluent. This gave 97 mg of the title compound. HPLC-MS: log P=1.75; mass (m/z): 290.1 (M+H)⁺; 1H NMR (DMSO-D₆) 1.41 (s, 9H), 2.23 (s, 3H), 5.31 (s, 2H), 6.11 (br. s, 1H), 7.41-7.43 (m, 1H), 8.75-8.76 (m, 2H), 9.37 (br. s, 1H).

### Stage 4: 5-Methyl-1-(pyrimidin-2-ylmethyl)-1H-pyrazole-3-amine

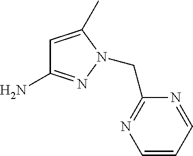

A mixture of tert-butyl[5-methyl-1-(pyrimidin-2-ylmethyl)-1H-pyrazol-3-yl]carbamate (37 mg) 4 N HCl solution in 1,4-dioxane (4 ml) was stirred at room temperature for 12 h and then concentrated under reduced pressure. The residue was admixed with sat. NaHCO₃ solution and extracted with dichloromethane. The organic phase was dried over sodium sulphate, filtered and concentrated under reduced pressure. This left 33 mg of the crude product, which was used without further purification for the next step. HPLC-MS: log P<−0.23; mass (m/z): 190.1 (M+H)⁺; 1H NMR (DMSO-D₆) 2.16 (s, 3H), 4.37 (s, 2H), 5.15 (s, 2H), 5.25 (s, 1H), 7.38-7.41 (m, 1H), 8.74-8.75 (m, 2H).

### Stage 5: 5-Fluoro-N-[5-methyl-1-(pyrimidin-2-ylmethyl)-1H-pyrazol-3-yl]nicotinamide

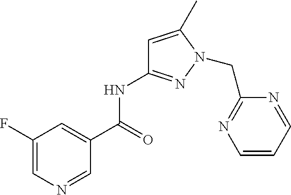 [SMILES: Cc1cc(NC(=O)c2cncc(F)c2)nn1Cc1ncccn1]

A solution of 5-fluoronicotinic acid (112 mg) in 3 ml of 1,4-dioxane was admixed with 1-ethyl-3-(3-dimethylaminopropyl)carbodiimide hydrochloride (183 mg), and the mixture was stirred at room temperature for 30 minutes. Subsequently, a solution of 5-methyl-1-(pyrimidin-2-ylmethyl)-1H-pyrazole-3-amine (166 mg) in 2 ml of 1,4-dioxane was added and the resulting reaction mixture was stirred at room temperature for 18 h. The mixture was concentrated under reduced pressure, the residue was admixed with water and extracted with ethyl acetate, and the organic phase was dried over sodium sulphate, filtered and concentrated under reduced pressure. This gave 207 mg of the title compound. HPLC-MS: log P=1.11; mass (m/z): 313.1 (M+H)⁺; 1H NMR (DMSO-D₆) 2.32 (s, 3H), 5.42 (s, 2H), 6.52 (s, 1H), 7.42-7.45 (m, 1H), 8.19-8.22 (m, 1H), 8.73-8.74 (m, 1H), 8.77-8.78 (m, 2H), 8.97 (s, 1H), 11.06 (s, 1H).

### Synthesis Example 13

### N-[5-Methyl-1-(pyrimidin-2-yl)-1H-pyrazol-3-yl]nicotinamide (Compound I-1-23 in Table 1)

### Stage 1: 2-(5-Methyl-3-nitro-1H-pyrazol-1-yl)pyrimidine (by Route A-1)

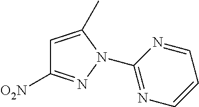

A solution of 5-methyl-3-nitro-1H-pyrazole (450 mg) in 30 ml of acetonitrile was admixed with potassium carbonate (733 mg) and 2-chloropyrimidine (608 mg), and the mixture was stirred at 90° C. for 12 h. The mixture was concentrated under reduced pressure, the residue was admixed with water and extracted with ethyl acetate, and the organic phase was dried over sodium sulphate, filtered and concentrated under reduced pressure. The crude product was stirred with n-hexane, filtered off with suction and dried under reduced pressure. This gave 539 mg of the title compound. HPLC-MS: log P=1.41; mass (m/z): 206.1 (M+H)⁺; 1H NMR (DMSO-D₆) 2.65 (s, 3H), 7.07 (s, 1H), 7.65-7.68 (m, 1H), 8.99-9.00 (m, 2H).

### Stage 2: 5-Methyl-1-(pyrimidin-2-yl)-1H-pyrazole-3-amine

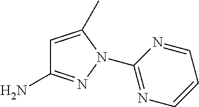

A solution of 2-(5-methyl-3-nitro-1H-pyrazol-1-yl)pyrimidine (539 mg) in ethanol was admixed with Pd/C (10%, 53 mg) and hydrogenated under a hydrogen atmosphere at 1 bar and room temperature for 18 h. The reaction solution was subsequently filtered through a silica gel pad and concentrated under reduced pressure. This gave 419 mg of the title compound. HPLC-MS: log P=0.5; mass (m/z): 176.1 (M+H)⁺; 1H NMR (DMSO-D₆) 2.52 (s, 3H), 4.87 (br. s, 2H), 5.65 (s, 1H), 7.19-7.21 (m, 1H), 8.68-8.70 (m, 2H).

### Stage 3: N-[5-Methyl-1-(pyrimidin-2-yl)-1H-pyrazol-3-yl]nicotinamide

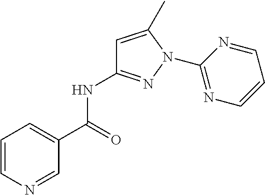 [SMILES: Cc1cc(NC(=O)c2cccnc2)nn1-c1ncccn1]

A solution of nicotinic acid (140 mg) in 2 ml of 1,4-dioxane was admixed with 1-ethyl-3-(3-dimethylaminopropyl)carbodiimide hydrochloride (262 mg), and the mixture was stirred at room temperature for 30 minutes. Subsequently, a solution of 5-methyl-1-(pyrimidin-2-yl)-1H-pyrazole-3-amine (200 mg) in 0.5 ml of 1,4-dioxane was added and the resulting reaction mixture was stirred at room temperature for 72 h. The mixture was concentrated under reduced pressure, the residue was admixed with water and extracted with ethyl acetate, and the organic phase was dried over sodium sulphate, filtered and concentrated under reduced pressure. The crude product was treated with acetonitrile in an ultrasound bath, and the precipitate was filtered off with suction and dried under reduced pressure. This gave 163 mg of the title compound. HPLC-MS: log P=1.14; mass (m/z): 281.1 (M+H)⁺; 1H NMR (DMSO-D₆) 2.66 (s, 3H), 6.83 (s, 1H), 7.41-7.43 (m, 1H), 7.49-7.53 (m, 1H), 8.35-8.38 (m, 1H), 8.72-8.74 (m, 1H), 8.84-8.85 (m, 2H), 9.17 (s, 1H), 11.26 (s, 1H).

### Synthesis Example 14

### N-Methyl-N-[5-methyl-1-(pyrimidin-2-yl)-1H-pyrazol-3-yl]nicotinamide (Compound I-1-25 in Table 1)

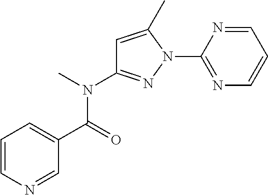 [SMILES: Cc1cc(N(C)C(=O)c2cccnc2)nn1-c1ncccn1]

A solution of N-[5-methyl-1-(pyrimidin-2-yl)-1H-pyrazol-3-yl]nicotinamide (154 mg) in 3 ml of tetrahydrofuran was admixed at 0° C. with sodium hydride (36 mg) and stirred at 0° C. for 1 h. Subsequently, iodomethane (155 mg) was added dropwise and the mixture was stirred at room temperature for 12 h. The mixture was diluted with ethyl acetate and washed with water, the aqueous phase was extracted with ethyl acetate, and the combined organic phases were dried over sodium sulphate, filtered and concentrated under reduced pressure. The residue was chromatographed by preparative HPLC using the mobile phase water/acetonitrile (gradient=40 min from 10% acetonitrile in water to 100% acetonitrile). This gave 16 mg of the title compound. HPLC-MS: log P=1.02; mass (m/z): 295.1 (M+H)⁺; 1H NMR (DMSO-D₆) 2.53 (s, 3H), 3.43 (s, 3H), 6.11 (br. s, 1H), 7.30-7.33 (m, 2H), 7.79-7.82 (m, 1H), 8.56-8.67 (m, 2H), 8.76-8.77 (m, 2H).

### Synthesis Example 15

### N-[5-Methyl-2-(pyridin-2-yl)-2H-1,2,3-triazol-4-yl]nicotinamide (Compound I-3-1 in Table 3)

### Stage 1-6: 5-Methyl-2-(pyridin-2-yl)-2H-1,2,3-triazol-4-amine

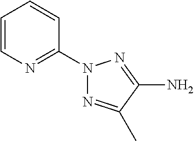

A mixture of 0.1 mol of 2-hydrazinopyridine and 0.1 mol of ethyl (2Z)-2-(hydroxyimino)-3-oxobutanoate in 100 ml propan-2-ol was stirred at room temperature for 1 h. Subsequently, the mixture was filtered, the filtrate was admixed with 100 ml of water and the mixture was left to stand overnight. The precipitated solids were filtered off with suction and dried (60% yield). The ethyl (2Z)-2-(hydroxyimino)-3-[2-(pyridin-2-yl)hydrazino]butanoate obtained was heated together with an excess of acetic anhydride to 120° C. for 1 h. The mixture was concentrated under reduced pressure and the resulting crude product was used without further purification (60% yield). Ethyl 5-methyl-2-(pyridin-2-yl)-2H-1,2,3-triazole-4-carboxylate obtained was heated together with hydrazine hydrate in ethanol under reflux for 4 h. The reaction mixture was cooled and the resulting solids were filtered off, washed with water and dried. 5-Methyl-2-(pyridin-2-yl)-2H-1,2,3-triazole-4-carbohydrazide was obtained with 75% yield. The hydrazide was subsequently suspended in acetic acid, an aqueous solution of sodium nitrite was added at 10° C. and the mixture was stirred for a while. The resulting solids were filtered off with suction, washed with water and dried under reduced pressure (65% yield). The 5-methyl-2-(pyridin-2-yl)-2H-1,2,3-triazole-4-carbonyl azide thus obtained was subsequently heated under reflux in propan-2-ol for 2 h until no further evolution of gas was observed. The mixture was concentrated under reduced pressure and the resulting crude product was used in the subsequent stage without further purification (90% yield). The isopropyl[5-methyl-2-(pyridin-2-yl)-2H-1,2,3-triazol-4-yl]carbamate thus obtained was heated in a mixture of acetic acid and conc. hydrochloric acid for 10 h. The mixture was concentrated under reduced pressure, the resulting crude product was treated with aqueous sodium carbonate solution, and the solids were filtered off with suction and washed with water. Recrystallization from hexane gave 5-methyl-2-(pyridin-2-yl)-2H-1,2,3-triazol-4-amine in 60% yield. HPLC-MS: log P=0.74; mass (m/z): 176.1 (M+H)⁺; 1H NMR (DMSO-D₆) 2.20 (s, 3H), 5.50 (br. s, 2H), 7.20-7.23 (m, 1H), 7.67-7.69 (m, 1H), 7.85-7.89 (m, 1H), 8.37-8.39 (m, 1H).

### Stage 7: N-[5-Methyl-2-(pyridin-2-yl)-2H-1,2,3-triazol-4-yl]nicotinamide

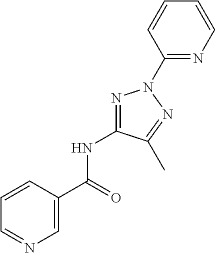 [SMILES: Cc1nn(-c2ccccn2)nc1NC(=O)c1cccnc1]

A solution of nicotinic acid (70 mg) in 12 ml of dichloromethane was admixed with 1-ethyl-3-(3-dimethylaminopropyl)carbodiimide hydrochloride (330 mg) and 4-(dimethylamino)pyridine (209 mg), and the mixture was stirred at room temperature for 15 minutes. Subsequently, 5-methyl-2-(pyridin-2-yl)-2H-1,2,3-triazole-4-amine (100 mg) was added and the resulting reaction mixture was stirred at room temperature for 18 h. The mixture was admixed with aqueous sodium dihydrogenphosphate solution, the phases were separated, and the aqueous phase was extracted with dichloromethane, dried over sodium sulphate and concentrated under reduced pressure. The residue was chromatographed on a preparative HPLC with water/acetonitrile as eluent. This gave 70 mg of the title compound. HPLC-MS: log P=1.08; mass (m/z): 281.1 (M+H)⁺; 1H NMR (DMSO-D₆) 2.32 (s, 3H), 7.44-7.47 (m, 1H), 7.58-7.62 (m, 1H), 7.93-7.95 (m, 1H), 8.03-8.07 (m, 1H), 8.36-8.38 (m, 1H), 8.54-8.55 (m, 1H), 8.80-8.81 (m, 1H), 9.18 (s, 1H), 11.11 (s, 1H).

### Synthesis Example 16

### N-(2-{2-Oxo-2-[(1-phenylethyl)amino]ethyl}-2H-tetrazol-5-yl)nicotinamide (Compound I-4-6 in Table 4)

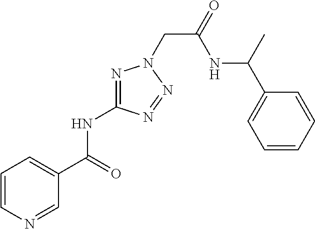 [SMILES: CC(NC(=O)Cn1nnc(NC(=O)c2cccnc2)n1)c1ccccc1]

A solution of 2-(5-amino-2H-tetrazol-2-yl)-N-(1-phenylethyl)acetamide (250 mg) in 4 ml of acetonitrile was admixed with pyridine (0.2 ml) and nicotinoyl chloride hydrochloride (180 mg). Subsequently, the reaction mixture was heated to 120° C. in a microwave (CEM Discover-S, 300W) for 1 h, and concentrated, and the residue was stirred with 10 ml of water. The resulting precipitate was filtered off with suction, washed with 5 ml of methyl tert-butyl ether/cyclohexane and dried under reduced pressure. This gave 350 mg of the title compound. HPLC-MS: log P=1.34; mass (m/z): 352.2 (M+H)⁺; 1H NMR (399.95 MHz, DMSO-D₆): 11.716 (5.7); 9.156 (4.0); 9.151 (4.1); 9.030 (2.2); 9.011 (2.3); 8.880 (2.7); 8.868 (2.7); 8.821 (2.7); 8.818 (2.9); 8.809 (2.9); 8.806 (2.9); 8.480 (0.6); 8.460 (1.3); 8.441 (0.7); 8.397 (1.5); 8.392 (2.2); 8.388 (1.6); 8.377 (1.7); 8.372 (2.4); 8.368 (1.6); 7.973 (1.7); 7.957 (2.1); 7.954 (2.1); 7.938 (1.5); 7.639 (2.0); 7.627 (2.1); 7.619 (2.0); 7.607 (2.0); 7.361 (16.0); 7.354 (8.6); 7.347 (8.2); 7.331 (1.8); 7.286 (0.5); 7.274 (1.2); 7.267 (1.6); 7.260 (1.7); 7.253 (1.9); 7.245 (1.4); 7.239 (1.0); 7.232 (0.6); 5.495 (12.6); 5.146 (0.4); 4.973 (0.4); 4.955 (1.6); 4.937 (2.4); 4.918 (1.7); 4.901 (0.5); 4.137 (0.3); 4.130 (0.3); 4.085 (0.4); 4.062 (0.4); 4.027 (0.4); 4.007 (0.4); 3.988 (0.4); 3.918 (0.4); 3.908 (0.4); 3.862 (0.3); 3.843 (0.3); 3.837 (0.3); 3.820 (0.3); 3.793 (0.4); 2.507 (35.7); 2.503 (45.5); 2.499 (36.0); 2.330 (0.3); 1.425 (9.9); 1.407 (10.0); 1.379 (0.6); 0.000 (28.5).

### Synthesis Example 17

### N-{2-[5-(Trifluoromethyl)pyridin-2-yl]-2H-1,2,3-triazol-4-yl}nicotinamide (Compound I-3-14 in Table 3)

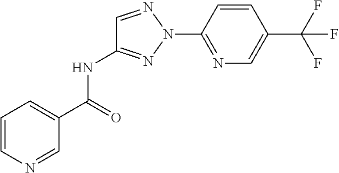 [SMILES: O=C(Nc1cnn(-c2ccc(C(F)(F)F)cn2)n1)c1cccnc1]

A solution of 2-[5-(trifluoromethyl)pyridin-2-yl]-2H-1,2,3-triazole-4-amine (217 mg) in dichloromethane (6 ml) was admixed with silver cyanide (317 mg) and nicotinoyl chloride hydrochloride (169 mg), and the resulting mixture was stirred at room temperature for six days. Filtration of the reaction mixture through kieselguhr, washing through with acetonitrile and methanol, and chromatography on a preparative HPLC with water/acetonitrile as eluent gave 31 mg of the title compound. HPLC-MS: log P=1.91; mass (m/z): 335.0 (M+H)⁺; 1H NMR (DMSO-D₆) 7.58-7.61 (m, 1H), 8.17-8.19 (m, 1H), 8.40-8.49 (m, 2H), 8.58 (s, 1H), 8.79-8.81 (m, 1H), 8.99 (s, 1H), 9.21-9.22 (m, 1H), 12.08 (s, 1H).

### Synthesis Example 18

### N-[1-(2-Chlorobenzyl)-1H-1,2,4-triazol-3-yl]nicotinamide (Compound I-2-7 in Table 2)

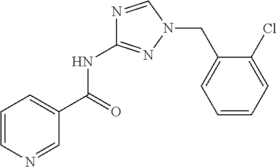 [SMILES: O=C(Nc1ncn(Cc2ccccc2Cl)n1)c1cccnc1]

A solution of 1-(2-chlorobenzyl)-1H-1,2,4-triazole-3-amine (202 mg) in dichloromethane (6 ml) was admixed with silver cyanide (324 mg) and nicotinoyl chloride hydrochloride (172 mg), and the resulting mixture was stirred at room temperature for four days. Filtration of the reaction mixture and chromatography on a preparative HPLC with water/acetonitrile as eluent gave 82 mg of the title compound. HPLC-MS: log P=1.22; mass (m/z): 314.0 (M)⁺; 1H NMR (DMSO-D₆) 5.50 (s, 2H), 7.28-7.30 (m, 1H), 7.35-7.42 (m, 2H), 7.49-7.54 (m, 2H), 8.25-8.27 (m, 1H), 8.62 (s, 1H), 8.73-8.75 (m, 1H), 9.06-9.07 (m, 1H), 10.99 (s, 1H).

### Synthesis Example 19

### N-{1-[(Ethylsulphanyl)methyl]-5-methyl-1H-pyrazol-3-yl}nicotinamide (Compound I-1-242 in Table 1)

### Stage 1: 1-[(Ethylsulphanyl)methyl]-5-methyl-3-nitro-1H-pyrazole (by Route A-1)

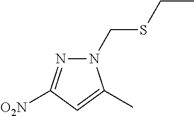

A solution of 1-(chloromethyl)-5-methyl-3-nitro-1H-pyrazole (2.00 g) in 50 ml of acetonitrile was admixed with sodium ethanethiolate (1.10 g) and stirred at 90° C. for 12 hours. More sodium ethanethiolate (0.5 g) was added and the mixture was stirred at 90° C. for a further 8 hours. The reaction mixture was concentrated under reduced pressure and purified by column chromatography on silica gel with cyclohexane/ethyl acetate (2:1) as eluent. This gave 1.00 g of the title compound. HPLC-MS: log P=2.21; mass (m/z): 202.1 (M+H)⁺; 1H NMR (DMSO-D₆) 1.14-1.18 (t, 3H), 2.39 (s, 3H), 2.58-2.63 (q, 2H), 5.41 (s, 2H), 6.91 (s, 1H).

### Stage 2: 1-[(Ethylsulphanyl)methyl]-5-methyl-1H-pyrazole-3-amine

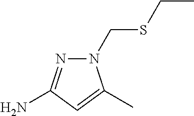

A solution of 1-[(ethylsulphanyl)methyl]-5-methyl-3-nitro-1H-pyrazole (980 mg) in methanol (60 ml) was admixed with Pd/C (10%, 100 mg) and hydrogenated under a hydrogen atmosphere at 10 bar and room temperature for 18 h. The reaction solution was subsequently filtered through Celite and concentrated under reduced pressure. This gave 830 mg of the title compound. HPLC-MS: log P=0.63; mass (m/z): 172.0 (M+H)⁺; 1H NMR (DMSO-D₆) 1.14-1.17 (t, 3H), 2.14 (s, 3H), 2.56-2.61 (q, 2H), 4.50 (br. s, 2H), 4.95 (s, 2H), 5.28 (s, 1H).

### Stage 3: N-{1-[(Ethylsulphanyl)methyl]-5-methyl-1H-pyrazol-3-yl}nicotinamide

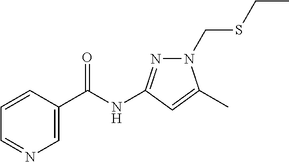 [SMILES: CCSCn1nc(NC(=O)c2cccnc2)cc1C]

A solution of 1-[(ethylsulphanyl)methyl]-5-methyl-1H-pyrazole-3-amine (500 mg) in dichloromethane (40 ml) was admixed with silver cyanide (977 mg) and nicotinoyl chloride hydrochloride (519 mg), and the resulting mixture was stirred at room temperature for two days. Filtration of the reaction mixture and purification of the crude product by column chromatography on silica gel with ethyl acetate/ethanol (98:2) as eluent gave 580 mg of the title compound. HPLC-MS: log P=1.49; mass (m/z): 277.1 (M+H)+; 1H NMR (DMSO-D₆) 1.16-1.19 (t, 3H), 2.32 (s, 3H), 2.59-2.65 (q, 2H), 5.19 (s, 2H), 6.53 (s, 1H), 7.49-7.53 (m, 1H), 8.30-8.33 (m, 1H), 8.71-8.72 (m, 1H), 9.10-9.11 (m, 1H), 11.05 (s, 1H).

### Synthesis Example 20

### N-{1-[(Ethylsulphonyl)methyl]-5-methyl-1H-pyrazol-3-yl}nicotinamide (Compound I-1-246 in Table 1)

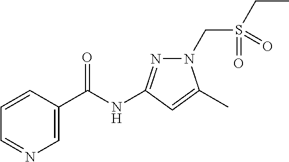 [SMILES: CCS(=O)(=O)Cn1nc(NC(=O)c2cccnc2)cc1C]

A solution of N-{1-[(ethylsulphanyl)methyl]-5-methyl-1H-pyrazol-3-yl}nicotinamide (425 mg) in 15 ml of dichloromethane was admixed with meta-chloroperbenzoic acid (77%, 448 mg), and the resulting mixture was stirred at room temperature overnight. The crude product was purified by column chromatography on silica gel with ethyl acetate/ethanol (6:4) as eluent. 105 mg of the title compound and 300 mg of N-{1-[(ethylsulphinyl)methyl]-5-methyl-1H-pyrazol-3-yl}nicotinamide were obtained. HPLC-MS: log P=0.83; mass (m/z): 309.1 (M+H)⁺; 1H NMR (DMSO-D₆) 1.24-1.28 (t, 3H), 2.36 (s, 3H), 3.16-3.22 (q, 2H), 5.60 (s, 2H), 6.60 (s, 1H), 7.50-7.54 (m, 1H), 8.30-8.32 (m, 1H), 8.72-8.74 (m, 1H), 9.10 (s, 1H), 11.12 (s, 1H).

Further novel 1H-pyrazole-3-amines of the formula (II-1) were obtained by or in analogy to the above-described synthesis examples.

### Compounds of the Formula (II-1)

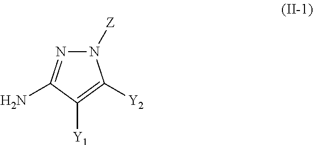

in which Y₁, Y₂ and Z are each defined as specified in the table below.

| Compd. | | | | log P |
|---|---|---|---|---|
| No. | Y₁ | Y₂ | Z | (acid)¹ |
| II-1-1 | H | cyclopropyl | (6-chloropyridin-3-yl)methyl | 1.21 |
| II-1-2 | H | cyclopropyl | benzyl | 1.36 |
| II-1-3 | H | H | (6-chloropyridin-3-yl)methyl | 0.56 |
| II-1-4 | H | cyclopropyl | prop-2-yn-1-yl | 0.63 |
| II-1-5 | H | methyl | (6-chloropyridin-3-yl)methyl | 0.77 |
| II-1-6 | H | methyl | pyrazin-2-yl | 0.8 |
| II-1-7 | H | H | 1,3-difluoropropan-2-yl | −0.09 |
| II-1-8 | H | H | [3-chloro-5-(trifluoromethyl)pyridin-2-yl]methyl | 1.36 |
| II-1-9 | H | methyl | (4,6-dimethylpyrimidin-2-yl)methyl | 0.42 |
| II-1-10 | H | methyl | 1,3-thiazol-2-ylmethyl | 0.19 |
| II-1-11 | H | methyl | 3-(trifluoromethoxy)benzyl | 1.39 |
| II-1-12 | H | methyl | (4,6-dimethoxypyrimidin-2-yl)methyl | 0.81 |
| II-1-13 | H | methyl | 2-chloroethyl | 0.13 |
| II-1-14 | 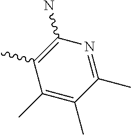 | | methyl | −0.12 |
| II-1-16 | H | methyl | (4-methylpyrimidin-2-yl)methyl | 0.02 |
| II-1-17 | H | ethyl | prop-2-yn-1-yl | 0.46 |
| II-1-18 | H | methyl | pyridin-4-ylmethyl | 0.45 |
| II-1-19 | H | ethyl | (6-chloropyridin-3-yl)methyl | 1.11 |
| II-1-20 | H | methyl | prop-2-yn-1-yl | −0.15 |
| II-1-23 | H | methyl | pyrimidin-2-ylmethyl | <−0.23 |
| II-1-24 | H | methyl | (3-chloropyridin-2-yl)methyl | 0.6 |
| II-1-25 | H | methyl | (3-cyanopyridin-2-yl)methyl | 0.21 |
| II-1-26 | bro- | difluoromethoxy | methyl | — |
| | mine | | | |
| II-1-27 | H | methyl | difluoromethyl | 0.47 |
| II-1-28 | H | methyl | pyridin-3-ylmethyl | <−0.23 |
| II-1-30 | H | bromine | pyrimidin-2-ylmethyl | 0.56 |
| II-1-31 | H | methyl | 3-chloropyridin-2-yl | 0.79 |
| II-1-33 | H | methyl | ethoxymethyl | 0.15 |
| II-1-34 | bro- | methyl | pyrimidin-2-ylmethyl | 0.92 |
| | mine | | | |
| II-1-35 | H | CF₃ | pyrimidin-2-ylmethyl | 1.06 |
| II-1-36 | H | Cl | pyrimidin-2-ylmethyl | 0.49 |
| II-1-37 | H | CH₃ | 2-cyanopropan-2-yl | 0.64 |
| II-1-38 | H | CH₃ | (3-methyl-1H-pyrazol-1-yl)methyl | 0.55 |
| II-1-39 | H | methyl | (5-methyl-1H-pyrazol-1-yl)methyl | 0.52 |
| II-1-40 | H | CHF₂ | methyl | 0.22 |
| II-1-41 | H | methyl | [5-(trifluoromethyl)-1H-pyrazol-1-yl]methyl | 1.26 |
| II-1-42 | H | methyl | [3-(trifluoromethyl)-1H-pyrazol-1-yl]methyl | 1.51 |
| II-1-43 | H | methyl | 1H-1,2,4-triazol-1-ylmethyl | −0.23 |
| II-1-44 | H | methyl | [(2E)-2-(cyanoimino)-1,3-thiazolidin-3-yl]methyl | 0.44 |
| II-1-45 | H | methyl | 1H-1,2,3-triazol-1-ylmethyl | <−0.23 |
| II-1-46 | H | methyl | (3,4-dimethyl-5-oxo-4,5-dihydro-1H-1,2,4-triazol-1- | <−0.23 |
| | | | yl)methyl | |
| II-1-47 | H | methyl | (4-cyclopropyl-3-methoxy-5-oxo-4,5-dihydro-1H- | 0.72 |
| | | | 1,2,4-triazol-1-yl)methyl | |
| II-1-48 | H | methyl | (2,4-dimethyl-3,5-dioxo-1,2,4-triazolidin-1-yl)methyl | 0.03 |
| II-1-49 | H | methyl | (4-cyclopropyl-3-methyl-5-oxo-4,5-dihydro-1H- | 0.43 |
| | | | 1,2,4-triazol-1-yl)methyl | |
| II-1-50 | H | methyl | (3,4-dicyclopropyl-5-oxo-4,5-dihydro-1H-1,2,4- | 0.98 |
| | | | triazol-1-yl)methyl | |
| II-1-51 | H | methyl | (pyrimidin-2-ylsulphanyl)methyl | 0.49 |
| II-1-52 | H | methyl | [(isopropylideneamino)oxy]methyl | 0.57 |
| II-1-53 | H | methyl | (2,2-dichlorocyclopropyl)methyl | 1.12 |
| II-1-54 | H | methyl | (1,3-dimethyl-5-oxo-1,5-dihydro-4H-1,2,4-triazol-4- | <−0.23 |
| | | | yl)methyl | |
| II-1-56 | H | methyl | 3,3-dichloroprop-2-en-1-yl | 1.13 |
| II-1-57 | H | methyl | (2-oxotetrahydropyrimidin-1(2H)-yl)methyl | <−0.23 |
| II-1-58 | H | methyl | 3-(2-chlorophenyl)-2-oxopropyl | 1.39 |
| II-1-59 | H | methyl | 3-methylbut-2-en-1-yl | 0.73 |
| II-1-60 | H | methyl | (methylsulphanyl)methyl | 0.14 |
| II-1-61 | H | methyl | (ethylsulphanyl)methyl | 0.63 |
| II-1-62 | H | CN | pyrimidin-2-ylmethyl | 0.20 |
| II-1-63 | H | methyl | (1-methyl-1H-1,2,4-triazol-5-yl)methyl | <−0.23 |
| II-1-64 | H | methyl | 1-cyanoethyl | 0.02 |

### NMR Data for Selected Examples²⁾

Compound No. II-1-1, solvent: [DMSO], spectrometer: 399.95 MHz
8.668 (0.5); 8.623 (0.4); 7.942 (0.3); 7.852 (0.4); 7.832 (0.4); 7.639 (0.4); 7.626 (0.4); 7.370 (0.4); 7.354
(0.4); 5.901 (0.5); 5.876 (0.4); 5.807 (1.1); 5.772 (0.4); 5.559 (0.7); 5.501 (2.0); 5.448 (0.6); 5.422 (0.4);
5.353 (0.8); 3.904 (4.0); 3.646 (0.4); 3.508 (0.4); 3.167 (16.0); 2.512 (24.5); 2.508 (48.8); 2.503 (63.9);
2.499 (47.6); 2.494 (24.4); 2.334 (0.3); 2.330 (0.4); 2.325 (0.4); 2.052 (0.4); 2.039 (0.6); 2.027 (0.4);
2.018 (0.5); 2.006 (0.4); 1.993 (0.4); 1.003 (0.5); 0.996 (0.6); 0.986 (1.3); 0.980 (1.4); 0.970 (1.1); 0.965
(1.5); 0.959 (1.7); 0.949 (1.1); 0.935 (0.9); 0.873 (0.3); 0.678 (0.8); 0.668 (1.3); 0.663 (1.3); 0.656 (1.3);
0.650 (1.3); 0.639 (0.8); 0.622 (0.9); 0.615 (0.9); 0.000 (2.2)
Compound No. II-1-2, solvent: [DMSO], spectrometer: 399.95 MHz
8.111 (0.5); 8.107 (0.5); 7.375 (0.8); 7.368 (0.9); 7.352 (2.1); 7.331 (4.7); 7.327 (5.4); 7.323 (4.3); 7.320
(4.6); 7.310 (10.0); 7.307 (6.5); 7.291 (7.9); 7.266 (2.7); 7.254 (5.5); 7.242 (3.1); 7.236 (5.3); 7.221
(1.8); 7.218 (2.0); 7.203 (1.0); 7.197 (1.0); 7.161 (1.8); 7.157 (1.9); 7.137 (7.3); 7.133 (7.8); 7.116 (6.0);
7.080 (0.5); 7.066 (0.5); 7.049 (0.4); 6.139 (0.8); 6.114 (0.4); 5.389 (0.5); 5.362 (0.5); 5.350 (0.4); 5.294
(1.6); 5.280 (1.3); 5.275 (1.4); 5.223 (0.4); 5.211 (0.5); 5.168 (0.6); 5.110 (16.0); 5.080 (11.4); 4.796
(0.6); 4.564 (0.9); 4.281 (0.8); 4.266 (0.7); 3.903 (5.8); 3.329 (134.7); 3.174 (0.5); 3.163 (0.5); 2.680
(0.5); 2.675 (0.8); 2.671 (1.0); 2.666 (0.6); 2.511 (96.4); 2.506 (149.7); 2.502 (175.8); 2.497 (122.2);
2.492 (57.6); 2.337 (1.0); 2.333 (1.4); 2.328 (4.0); 2.205 (1.4); 1.935 (0.4); 1.912 (0.4); 1.899 (0.3);
1.891 (0.4); 1.884 (0.3); 1.878 (0.5); 1.864 (0.5); 1.856 (0.4); 1.845 (0.4); 1.765 (1.0); 1.752 (1.7); 1.744
(2.0); 1.740 (1.5); 1.732 (3.0); 1.723 (1.4); 1.719 (1.8); 1.711 (1.6); 1.698 (0.8); 1.298 (0.4); 1.258 (0.6);
1.236 (0.7); 1.037 (0.3); 1.002 (0.3); 0.996 (0.4); 0.985 (0.6); 0.981 (0.6); 0.970 (0.7); 0.964 (0.9); 0.955
(0.7); 0.949 (0.8); 0.943 (0.7); 0.934 (0.7); 0.928 (0.6); 0.920 (0.9); 0.910 (1.3); 0.903 (1.6); 0.897 (1.4);
0.889 (1.4); 0.884 (1.4); 0.853 (3.5); 0.842 (6.9); 0.837 (7.4); 0.832 (4.5); 0.827 (4.6); 0.821 (6.7); 0.816
(6.1); 0.806 (2.7); 0.789 (0.7); 0.779 (0.5); 0.769 (0.6); 0.754 (0.4); 0.717 (1.1); 0.710 (0.9); 0.696 (1.2);
0.691 (0.9); 0.684 (0.8); 0.678 (0.6); 0.671 (0.8); 0.664 (0.5); 0.657 (0.6); 0.651 (0.7); 0.645 (0.5); 0.628
(0.5); 0.610 (0.5); 0.597 (0.7); 0.587 (0.9); 0.582 (0.9); 0.574 (0.9); 0.569 (1.0); 0.558 (0.8); 0.547 (0.7);
0.541 (0.6); 0.534 (0.5); 0.510 (3.4); 0.500 (7.1); 0.497 (5.3); 0.494 (6.9); 0.487 (6.4); 0.482 (5.9); 0.472
(2.1); 0.455 (0.5); 0.447 (0.5); 0.442 (0.5); 0.436 (0.4); 0.008 (0.4); 0.000 (3.9)
Compound No. II-1-3, solvent: [DMSO], spectrometer: 399.95 MHz
8.723 (1.0); 8.646 (2.1); 8.202 (1.5); 8.183 (1.1); 7.993 (1.7); 7.987 (1.9); 7.924 (3.5); 7.889 (1.7); 7.882
(1.7); 7.862 (2.0); 7.785 (0.8); 7.615 (1.0); 7.553 (0.4); 7.532 (0.4); 7.313 (1.8); 7.293 (1.5); 7.197 (0.4);
7.069 (0.3); 6.310 (0.7); 6.305 (0.8); 6.283 (2.1); 6.268 (1.6); 6.175 (1.2); 6.148 (1.2); 5.441 (2.6); 5.415
(3.2); 5.363 (0.7); 5.339 (0.6); 5.288 (3.2); 5.253 (3.0); 3.903 (12.5); 3.507 (5.8); 3.489 (5.7); 3.476
(5.8); 3.463 (5.6); 3.388 (4.4); 3.378 (4.2); 3.371 (4.2); 3.365 (4.0); 3.354 (3.8); 3.167 (16.0); 2.676
(0.8); 2.672 (1.3); 2.667 (0.9); 2.512 (102.1); 2.507 (217.5); 2.503 (295.7); 2.498 (225.8); 2.494 (120.7);
2.334 (2.2); 2.329 (2.7); 2.325 (2.3); 1.954 (0.5); 1.557 (0.4); 1.470 (0.3); 1.450 (0.3); 1.434 (0.4); 1.336
(0.5); 1.298 (1.5); 1.258 (1.9); 1.249 (0.6); 1.235 (0.7); 1.215 (0.4); 0.891 (0.4); 0.873 (0.7); 0.855 (0.4);
0.000 (9.8); −0.009 (0.5)
Compound No. II-1-4, solvent: [DMSO], spectrometer: 399.95 MHz
8.124 (0.4); 8.121 (0.4); 6.438 (0.4); 6.120 (0.7); 6.103 (0.7); 5.058 (11.8); 5.031 (0.5); 4.975 (0.5);
4.938 (0.8); 4.933 (1.0); 4.929 (0.4); 4.916 (1.0); 4.912 (1.0); 4.719 (15.7); 4.715 (16.0); 4.675 (0.5);
4.516 (0.3); 4.489 (0.6); 4.485 (0.8); 4.471 (6.3); 3.384 (0.3); 3.367 (0.5); 3.322 (197.8); 3.266 (3.7);
3.262 (8.0); 3.258 (3.8); 3.230 (0.4); 3.224 (0.4); 3.220 (0.5); 3.216 (0.4); 2.616 (0.7); 2.613 (1.0); 2.610
(0.7); 2.522 (2.0); 2.519 (2.6); 2.516 (2.9); 2.507 (56.4); 2.504 (120.3); 2.501 (165.7); 2.498 (121.5);
2.495 (57.7); 2.388 (0.9); 2.385 (1.2); 2.382 (0.9); 1.947 (2.5); 1.895 (0.4); 1.884 (1.3); 1.788 (0.8);
1.780 (1.5); 1.774 (1.6); 1.766 (2.9); 1.758 (1.7); 1.752 (1.6); 1.744 (0.9); 1.336 (0.5); 1.298 (0.6); 1.258
(0.8); 1.235 (0.9); 1.225 (0.7); 1.040 (0.3); 1.033 (0.5); 1.029 (0.5); 1.019 (0.6); 1.015 (0.6); 1.008 (0.4);
0.976 (0.4); 0.969 (0.8); 0.965 (0.8); 0.959 (0.8); 0.955 (1.0); 0.952 (0.8); 0.945 (0.7); 0.941 (0.6); 0.925
(0.4); 0.901 (2.6); 0.894 (6.4); 0.890 (6.6); 0.887 (3.2); 0.884 (3.2); 0.880 (6.6); 0.876 (6.6); 0.870 (2.8);
0.865 (1.1); 0.861 (1.1); 0.857 (1.0); 0.853 (0.9); 0.846 (0.7); 0.742 (0.4); 0.636 (0.6); 0.624 (0.7); 0.616
(0.7); 0.608 (0.6); 0.604 (0.6); 0.571 (0.3); 0.562 (0.4); 0.546 (2.7); 0.539 (6.6); 0.536 (6.8); 0.531 (6.5);
0.527 (6.7); 0.520 (2.3); 0.498 (0.5); 0.451 (0.4); 0.442 (0.4); 0.005 (0.4); 0.000 (9.2)
Compound No. II-1-5, solvent: [DMSO], spectrometer: 399.95 MHz
8.572 (0.5); 8.553 (0.3); 7.704 (0.3); 7.683 (0.4); 7.662 (0.4); 7.655 (0.4); 7.641 (0.3); 7.545 (0.4); 7.525
(0.5); 7.519 (0.5); 7.498 (0.5); 7.488 (0.4); 7.310 (0.7); 7.192 (0.6); 7.184 (0.6); 7.064 (0.6); 5.953 (0.5);
5.347 (0.5); 5.317 (1.2); 5.289 (1.0); 5.273 (0.4); 5.240 (0.4); 5.169 (0.6); 4.876 (0.3); 3.903 (4.4); 3.421
(2.5); 3.414 (2.5); 3.388 (2.3); 3.372 (2.1); 3.339 (1.7); 3.167 (16.0); 2.676 (0.4); 2.672 (0.5); 2.667
(0.4); 2.525 (0.7); 2.520 (1.4); 2.511 (35.6); 2.507 (74.0); 2.502 (98.6); 2.498 (72.3); 2.493 (36.0); 2.338
(0.6); 2.334 (0.8); 2.329 (1.0); 2.325 (0.8); 2.320 (0.5); 2.303 (3.5); 2.295 (2.5); 2.273 (2.3); 2.266 (2.5);
2.223 (1.0); 2.207 (0.9); 2.198 (0.5); 2.187 (0.5); 2.169 (0.4); 2.141 (0.4); 2.105 (0.4); 1.298 (0.4); 1.258
(0.5); 0.000 (3.5)
Compound No. II-1-6, solvent: [DMSO], spectrometer: 399.95 MHz
9.304 (0.7); 9.301 (0.6); 9.086 (0.3); 8.941 (13.8); 8.939 (13.5); 8.737 (0.6); 8.730 (0.7); 8.657 (0.5);
8.654 (0.5); 8.651 (0.5); 8.647 (0.4); 8.522 (0.4); 8.515 (0.6); 8.510 (0.5); 8.507 (0.4); 8.372 (7.3); 8.366
(12.1); 8.363 (11.5); 8.349 (15.6); 8.343 (9.0); 6.791 (0.7); 6.789 (0.7); 5.669 (16.0); 5.279 (2.5); 3.904
(10.1); 3.508 (0.4); 3.333 (178.9); 3.169 (5.4); 2.703 (2.9); 2.676 (1.7); 2.672 (2.2); 2.667 (1.8); 2.662
(1.3); 2.656 (1.6); 2.646 (0.6); 2.635 (1.3); 2.624 (0.4); 2.615 (0.4); 2.609 (0.4); 2.586 (0.6); 2.573 (1.5);
2.543 (59.9); 2.525 (6.3); 2.512 (116.2); 2.507 (229.4); 2.503 (298.4); 2.498 (213.5); 2.494 (101.9);
2.380 (0.5); 2.338 (0.7); 2.334 (1.4); 2.329 (1.9); 2.325 (1.3); 2.320 (0.6); 1.298 (0.7); 1.259 (1.1); 1.249
(0.6); 1.234 (2.0); 0.854 (0.6); 0.843 (0.4); 0.836 (0.5); 0.813 (0.4); 0.008 (0.4); 0.000 (10.2); −0.009
(0.3)
Compound No. II-1-7, solvent: [DMSO], spectrometer: 399.95 MHz
7.314 (13.7); 7.308 (13.7); 7.297 (0.4); 5.561 (16.0); 5.555 (15.8); 4.817 (3.2); 4.800 (4.0); 4.793 (7.7);
4.775 (9.7); 4.761 (8.3); 4.749 (10.2); 4.737 (3.5); 4.725 (3.9); 4.697 (2.9); 4.679 (4.6); 4.673 (6.8);
4.656 (9.7); 4.643 (8.0); 4.634 (5.6); 4.631 (10.5); 4.623 (2.5); 4.619 (3.7); 4.611 (2.5); 4.607 (5.4);
4.601 (0.9); 4.591 (2.2); 4.580 (2.6); 4.574 (2.6); 4.568 (1.3); 4.563 (3.8); 4.557 (1.3); 4.551 (2.0); 4.545
(1.8); 4.537 (1.5); 4.533 (1.5); 4.525 (0.8); 4.520 (2.0); 4.514 (0.7); 4.508 (0.9); 4.503 (0.7); 4.491 (0.4);
4.068 (0.6); 4.050 (0.6); 4.032 (0.4); 3.919 (2.0); 3.911 (1.9); 2.535 (0.4); 2.508 (0.3); 2.229 (0.3); 2.129
(0.5); 1.971 (1.9); 1.962 (1.1); 1.956 (1.7); 1.950 (7.9); 1.944 (13.9); 1.938 (18.3); 1.932 (12.5); 1.925
(6.4); 1.474 (1.4); 1.440 (1.6); 1.221 (0.5); 1.203 (1.0); 1.185 (0.5); 1.145 (0.6); 0.008 (1.3); 0.000
(32.6); −0.009 (1.0)
Compound No. II-1-8, solvent: [DMSO], spectrometer: 399.95 MHz
8.899 (4.9); 8.896 (4.8); 8.472 (4.9); 8.469 (4.7); 7.464 (7.7); 7.459 (7.5); 5.425 (8.8); 5.420 (8.7); 5.357
(16.0); 4.568 (6.4); 3.332 (2.3); 2.512 (4.8); 2.507 (9.5); 2.503 (12.3); 2.498 (8.8); 2.494 (4.2); 0.000
(2.1)
Compound No. II-1-9, solvent: [DMSO], spectrometer: 399.95 MHz
7.130 (2.0); 5.243 (2.1); 5.030 (5.0); 4.360 (2.2); 3.325 (3.7); 2.519 (0.4); 2.510 (3.0); 2.506 (5.9); 2.501
(7.8); 2.497 (5.6); 2.492 (2.7); 2.382 (0.4); 2.362 (16.0); 2.179 (7.4); 0.000 (5.5)
Compound No. II-1-10, solvent: [DMSO], spectrometer: 399.95 MHz
7.771 (0.7); 7.764 (0.8); 7.731 (4.5); 7.693 (0.7); 7.686 (0.7); 7.647 (4.1); 7.221 (0.7); 7.093 (0.7); 6.965
(0.7); 5.508 (0.9); 5.485 (0.3); 5.447 (0.4); 5.389 (0.4); 5.359 (0.8); 5.321 (5.8); 5.294 (10.3); 4.793
(0.7); 3.903 (5.5); 3.329 (128.7); 3.208 (0.6); 3.168 (1.0); 2.675 (1.0); 2.671 (1.3); 2.666 (0.9); 2.523
(2.8); 2.506 (186.6); 2.501 (232.8); 2.497 (171.3); 2.360 (0.4); 2.328 (1.9); 2.324 (1.5); 2.309 (0.9);
2.277 (1.6); 2.154 (16.0); 2.101 (0.8); 2.066 (1.0); 2.038 (0.7); 1.990 (0.3); 1.926 (0.5); 1.820 (0.3);
1.258 (0.3); 1.235 (0.5); 0.000 (6.1)
Compound No. II-1-11, solvent: [DMSO], spectrometer: 399.95 MHz
7.478 (0.4); 7.471 (1.8); 7.451 (3.7); 7.431 (2.9); 7.411 (0.9); 7.390 (0.6); 7.317 (0.4); 7.299 (0.9); 7.280
(1.0); 7.250 (2.0); 7.225 (2.0); 7.210 (1.4); 7.206 (1.5); 7.189 (0.9); 7.141 (2.5); 7.121 (2.2); 7.107 (0.4);
7.082 (3.3); 7.060 (0.6); 7.050 (0.4); 7.016 (0.7); 6.441 (0.4); 5.360 (0.4); 5.349 (0.4); 5.323 (0.7); 5.310
(0.7); 5.295 (0.7); 5.271 (0.7); 5.268 (0.6); 5.260 (0.6); 5.249 (0.6); 5.243 (0.5); 5.234 (0.5); 5.188 (0.3);
5.175 (0.4); 5.140 (2.5); 5.113 (1.9); 5.075 (8.0); 4.011 (0.8); 3.995 (0.3); 3.440 (0.3); 3.426 (0.4); 3.393
(0.6); 3.378 (0.9); 3.324 (943.5); 3.268 (0.9); 3.241 (0.4); 2.710 (0.5); 2.692 (0.5); 2.675 (8.2); 2.670
(11.2); 2.666 (8.1); 2.589 (0.7); 2.572 (0.9); 2.540 (7.9); 2.510 (699.4); 2.505 (1346.2); 2.501 (1747.0);
2.497 (1277.8); 2.492 (632.1); 2.371 (0.9); 2.332 (8.7); 2.328 (11.6); 2.323 (8.6); 2.301 (0.7); 2.281
(0.8); 2.261 (0.5); 2.248 (0.5); 2.245 (0.4); 2.242 (0.4); 2.227 (0.5); 2.195 (0.5); 2.186 (0.5); 2.179 (0.4);
2.146 (0.4); 2.132 (0.4); 2.109 (0.4); 2.097 (0.4); 2.091 (0.5); 2.075 (0.3); 2.062 (0.3); 2.047 (0.4); 2.027
(0.4); 2.006 (0.8); 1.975 (16.0); 1.919 (3.2); 1.882 (0.8); 1.848 (0.3); 1.829 (1.0); 1.814 (0.3); 1.760
(0.8); 1.734 (0.3); 1.667 (0.5); 1.559 (2.4); 1.505 (0.3); 1.459 (0.5); 1.440 (0.4); 1.433 (0.4); 1.297 (1.3);
1.258 (2.6); 1.236 (9.3); 1.147 (0.3); 1.131 (0.3); 0.869 (0.8); 0.853 (2.0); 0.835 (0.9); 0.008 (1.5); 0.000
(32.7); −0.008 (1.4)
Compound No. II-1-12, solvent: [DMSO], spectrometer: 399.95 MHz
6.117 (2.6); 5.281 (1.6); 4.961 (3.7); 3.903 (0.7); 3.810 (16.0); 3.331 (26.7); 2.519 (0.4); 2.511 (6.9);
2.506 (14.1); 2.502 (18.6); 2.497 (13.4); 2.493 (6.4); 2.217 (5.7); 0.000 (0.6)
Compound No. II-1-13, solvent: [DMSO], spectrometer: 399.95 MHz
5.983 (0.7); 5.873 (2.3); 5.455 (0.7); 5.433 (1.3); 5.400 (1.4); 5.384 (2.0); 5.372 (1.5); 5.340 (1.7); 4.368
(2.9); 4.359 (5.6); 4.350 (3.4); 4.186 (2.9); 4.177 (3.8); 4.167 (2.9); 4.157 (3.8); 4.147 (6.1); 4.137 (4.1);
4.068 (4.1); 3.974 (5.2); 3.964 (7.6); 3.955 (5.0); 3.916 (4.2); 3.907 (6.4); 3.897 (4.8); 3.425 (4.5); 3.374
(3.8); 3.296 (1.5); 2.617 (1.2); 2.614 (1.9); 2.610 (1.3); 2.523 (0.9); 2.520 (1.3); 2.517 (0.6); 2.508
(106.3); 2.505 (238.7); 2.502 (338.9); 2.499 (251.3); 2.496 (121.8); 2.389 (2.1); 2.386 (2.7); 2.383 (2.1);
2.301 (16.0); 2.205 (7.5); 2.192 (2.6); 2.182 (12.1); 2.169 (6.7); 2.116 (7.9); 2.088 (5.8); 2.085 (4.3);
2.000 (2.0); 1.967 (1.5); 1.909 (2.6); 1.235 (0.9); 0.000 (5.4)
Compound No. II-1-14, solvent: [DMSO], spectrometer: 399.95 MHz
5.711 (3.1); 3.742 (16.0); 3.672 (0.8); 3.323 (53.0); 2.522 (0.4); 2.519 (0.4); 2.516 (0.4); 2.507 (11.3);
2.504 (24.8); 2.501 (34.1); 2.498 (24.9); 2.495 (11.7); 2.474 (0.7); 2.459 (11.4); 2.387 (14.2); 2.171
(0.5); 2.117 (10.9); 0.000 (1.0)
Compound No. II-1-16, solvent: [DMSO], spectrometer: 399.95 MHz
8.573 (3.3); 8.560 (3.3); 7.271 (2.3); 7.258 (2.2); 5.756 (0.8); 5.251 (3.8); 5.089 (9.3); 4.364 (3.8); 3.325
(1.9); 2.519 (0.4); 2.510 (4.7); 2.506 (9.7); 2.501 (12.8); 2.497 (9.2); 2.492 (4.3); 2.429 (16.0); 2.166
(13.2); 2.165 (13.8); 0.000 (2.7)
Compound No. II-1-17, solvent: [DMSO], spectrometer: 399.95 MHz
5.263 (6.6); 5.033 (0.5); 5.029 (0.5); 4.620 (9.7); 4.615 (9.9); 4.486 (3.7); 3.338 (194.5); 3.304 (0.3);
3.266 (2.2); 3.261 (4.6); 3.257 (2.1); 2.835 (0.4); 2.822 (0.4); 2.701 (0.4); 2.625 (0.5); 2.613 (0.6); 2.523
(2.2); 2.517 (1.1); 2.508 (12.7); 2.505 (23.2); 2.502 (31.3); 2.499 (24.4); 2.497 (14.1); 2.485 (1.9); 2.409
(0.4); 1.314 (0.3); 1.298 (0.4); 1.258 (0.6); 1.255 (0.5); 1.242 (1.0); 1.237 (0.6); 1.230 (0.5); 1.210 (0.5);
1.197 (1.0); 1.193 (0.4); 1.184 (0.6); 1.149 (7.7); 1.137 (16.0); 1.124 (7.5); 0.000 (1.3)
Compound No. II-1-18, solvent: [DMSO], spectrometer: 399.95 MHz
8.740 (0.3); 8.731 (0.5); 8.716 (0.4); 8.684 (0.3); 8.540 (0.7); 8.525 (0.8); 8.499 (2.4); 8.485 (2.5); 7.891
(0.4); 7.876 (0.4); 7.481 (0.5); 7.466 (0.5); 7.131 (0.5); 7.117 (0.4); 7.087 (0.3); 7.076 (0.4); 7.061 (0.4);
7.014 (3.1); 7.000 (3.0); 6.959 (0.3); 6.588 (0.4); 5.861 (0.4); 5.489 (0.4); 5.322 (4.3); 5.310 (0.8); 5.190
(0.8); 5.042 (8.1); 3.903 (5.6); 3.329 (101.5); 3.174 (0.9); 3.162 (0.8); 2.675 (0.5); 2.671 (0.7); 2.666
(0.5); 2.541 (0.3); 2.524 (1.8); 2.511 (46.8); 2.506 (93.4); 2.502 (122.4); 2.497 (88.3); 2.493 (42.7);
2.333 (0.6); 2.329 (0.8); 2.324 (0.6); 2.275 (0.8); 2.261 (1.4); 2.199 (0.4); 2.080 (16.0); 2.065 (0.7);
2.056 (0.4); 2.008 (0.4); 1.994 (0.4); 1.973 (0.4); 1.835 (2.1); 1.235 (0.3); 0.000 (3.9)
Compound No. II-1-19, solvent: [DMSO], spectrometer: 399.95 MHz
8.631 (0.9); 8.553 (0.6); 8.073 (0.5); 7.877 (0.3); 7.784 (0.5); 7.601 (0.5); 7.360 (0.4); 6.124 (0.5); 6.101
(0.5); 6.024 (0.9); 5.984 (0.4); 5.436 (0.8); 5.383 (2.1); 5.333 (0.6); 5.305 (0.6); 5.241 (0.9); 3.903 (5.8);
3.463 (6.3); 3.168 (16.0); 2.992 (0.5); 2.714 (0.6); 2.695 (1.7); 2.676 (2.7); 2.668 (1.6); 2.656 (1.9);
2.648 (1.2); 2.637 (1.3); 2.508 (80.9); 2.503 (107.0); 2.499 (81.9); 2.422 (0.7); 2.330 (0.9); 2.279 (0.5);
2.232 (0.3); 1.298 (0.5); 1.258 (0.6); 1.230 (1.0); 1.211 (1.8); 1.192 (1.9); 1.178 (2.7); 1.173 (2.9); 1.159
(4.7); 1.152 (3.3); 1.141 (2.9); 0.000 (1.6)
Compound No. II-1-20, solvent: [DMSO], spectrometer: 399.95 MHz
8.131 (0.4); 6.323 (0.5); 5.253 (4.0); 5.169 (0.4); 4.841 (0.9); 4.834 (1.0); 4.811 (0.3); 4.618 (7.0); 4.612
(7.6); 4.476 (1.1); 3.903 (9.1); 3.327 (214.3); 3.278 (3.2); 3.272 (5.3); 3.265 (3.1); 3.174 (1.0); 3.161
(1.0); 2.675 (0.6); 2.671 (0.9); 2.666 (0.6); 2.524 (1.2); 2.510 (62.4); 2.506 (128.7); 2.501 (171.3); 2.497
(126.4); 2.492 (63.7); 2.422 (2.1); 2.397 (1.0); 2.388 (1.1); 2.365 (1.2); 2.333 (1.4); 2.328 (1.6); 2.324
(1.3); 2.302 (0.9); 2.281 (0.9); 2.258 (2.4); 2.246 (1.6); 2.210 (0.8); 2.180 (0.8); 2.159 (1.0); 2.131
(16.0); 2.104 (1.2); 1.958 (1.3); 1.298 (0.4); 1.258 (0.5); 1.235 (0.7); 0.873 (0.4); 0.855 (0.4); 0.000 (5.5)
Compound No. II-1-23, solvent: [DMSO], spectrometer: 399.95 MHz
8.755 (7.5); 8.743 (7.5); 7.413 (1.7); 7.401 (3.3); 7.389 (1.6); 5.259 (4.4); 5.153 (10.9); 4.442 (0.3);
4.373 (4.3); 4.258 (0.6); 3.326 (2.7); 3.132 (0.5); 3.117 (0.3); 2.524 (0.5); 2.510 (7.6); 2.506 (14.4);
2.501 (18.4); 2.497 (12.9); 2.492 (6.0); 2.161 (16.0); 2.084 (1.3); 1.073 (0.4); 1.055 (0.8); 1.038 (0.4);
0.000 (5.0)
Compound No. II-1-24, solvent: [DMSO], spectrometer: 399.95 MHz
8.458 (0.3); 8.452 (2.1); 8.449 (2.3); 8.441 (2.2); 8.437 (2.1); 8.177 (1.4); 7.923 (2.1); 7.920 (2.1); 7.903
(2.3); 7.900 (2.2); 7.372 (2.0); 7.360 (2.0); 7.351 (1.8); 7.340 (1.8); 6.333 (0.4); 5.756 (2.6); 5.380 (0.9);
5.241 (4.4); 5.240 (4.4); 5.181 (11.0); 2.519 (0.5); 2.510 (7.3); 2.506 (14.9); 2.501 (19.6); 2.497 (14.0);
2.492 (6.6); 2.324 (0.4); 2.278 (1.3); 2.244 (0.4); 2.183 (16.0); 2.041 (0.4); 0.008 (0.5); 0.000 (13.9); −0.009
(0.4)
Compound II-1-25, solvent: [DMSO], spectrometer: 399.95 MHz
8.770 (2.2); 8.765 (2.3); 8.757 (2.1); 8.753 (2.1); 8.320 (2.0); 8.316 (2.1); 8.301 (2.2); 8.296 (2.1); 8.157
(1.3); 7.543 (2.0); 7.531 (2.0); 7.524 (1.9); 7.512 (1.8); 5.755 (0.5); 5.440 (0.5); 5.249 (4.5); 5.248 (4.6);
5.221 (11.1); 2.510 (6.6); 2.506 (13.4); 2.501 (17.6); 2.497 (12.5); 2.492 (5.8); 2.386 (0.4); 2.344 (0.8);
2.306 (0.5); 2.250 (16.0); 2.097 (0.5); 0.008 (0.4); 0.000 (11.0); −0.009 (0.3)
Compound No. II-1-27, solvent: [DMSO], spectrometer: 399.95 MHz
12.048 (0.3); 10.357 (1.1); 8.608 (0.9); 8.589 (0.8); 8.114 (2.9); 8.111 (2.8); 6.243 (1.6); 5.740 (1.5);
5.400 (3.7); 3.326 (2.4); 2.616 (0.5); 2.613 (0.6); 2.610 (0.5); 2.522 (1.1); 2.519 (1.4); 2.516 (1.6); 2.507
(34.2); 2.504 (71.8); 2.501 (98.4); 2.498 (72.1); 2.495 (34.0); 2.389 (0.4); 2.386 (0.6); 2.383 (0.4); 2.184
(13.9); 2.171 (6.4); 2.130 (16.0); 1.235 (1.0); 0.854 (0.3); 0.000 (4.1)
Compound II-1-28: 1H NMR (DMSO-D6) 1.90-1.93 (m, 3H), 5.02 (s, 2H), 5.37 (s, 1H),
7.24-7.27 (m, 1H), 7.43-7.46 (m, 1H), 8.36-8.37 (m, 1H), 8.44-8.45 (m, 1H).
Compound No. II-1-30, solvent: [DMSO], spectrometer: 399.95 MHz
8.943 (0.4); 8.811 (0.6); 8.799 (0.7); 8.782 (0.4); 8.771 (12.0); 8.758 (12.0); 7.436 (2.7); 7.424 (5.1);
7.412 (2.5); 6.782 (0.7); 5.612 (12.1); 5.602 (0.7); 5.262 (16.0); 4.795 (7.0); 4.636 (0.7); 4.622 (0.3);
4.490 (0.3); 3.395 (0.4); 3.380 (0.9); 3.366 (0.9); 3.351 (0.4); 3.323 (25.0); 3.089 (0.8); 3.074 (0.8);
2.671 (0.3); 2.524 (1.0); 2.511 (20.4); 2.506 (41.6); 2.502 (54.9); 2.497 (39.1); 2.492 (18.5); 2.328 (0.4);
1.790 (4.2); 0.008 (1.7); 0.000 (47.4); −0.009 (1.6)
Compound II-1-31: 1H NMR (CD3CN) 1.91 (s, 3H), 5.59 (s, 1H), 7.37-7.40 (m, 1H), 7.94-7.97 (m,
1H), 8.43-8.44 (m, 1H).
Compound No. II-1-33, solvent: [DMSO], spectrometer: 399.95 MHz
7.785 (0.4); 7.780 (0.5); 7.706 (0.5); 7.701 (0.5); 6.558 (0.4); 5.682 (0.6); 5.569 (1.4); 5.079 (1.5); 4.976
(0.7); 4.628 (1.3); 4.609 (3.0); 4.598 (2.1); 4.590 (2.0); 4.580 (2.1); 4.488 (0.8); 4.454 (0.6); 3.816 (0.4);
3.457 (3.3); 3.445 (6.4); 3.434 (6.9); 3.422 (4.8); 3.412 (4.3); 3.400 (5.1); 3.377 (7.4); 3.335 (10.1);
3.175 (3.9); 3.141 (1.9); 3.134 (2.8); 3.128 (2.1); 3.091 (1.4); 2.752 (0.8); 2.616 (1.2); 2.613 (1.7); 2.610
(1.2); 2.540 (0.6); 2.522 (3.3); 2.519 (4.3); 2.516 (4.5); 2.507 (88.3); 2.504 (190.7); 2.501 (263.2); 2.498
(192.3); 2.495 (90.7); 2.388 (1.5); 2.385 (2.0); 2.382 (1.6); 2.338 (2.1); 2.293 (0.8); 2.243 (1.4); 2.222
(1.0); 2.195 (1.9); 2.166 (1.5); 2.154 (1.4); 2.131 (1.8); 2.097 (2.6); 2.086 (3.0); 2.080 (3.2); 2.069 (2.5);
2.059 (4.0); 2.051 (3.6); 2.049 (5.4); 2.034 (2.3); 2.024 (4.2); 2.003 (2.5); 1.981 (14.1); 1.955 (4.7);
1.949 (3.1); 1.939 (2.4); 1.926 (2.5); 1.921 (2.1); 1.915 (1.9); 1.908 (3.0); 1.870 (1.8); 1.866 (1.8); 1.856
(1.3); 1.108 (0.5); 1.096 (0.4); 1.083 (0.4); 1.067 (8.3); 1.055 (16.0); 1.049 (1.3); 1.043 (8.4); 1.037
(2.0); 1.026 (1.2); 0.986 (0.5); 0.005 (0.4); 0.000 (11.4); −0.006 (0.4)
Compound No. II-1-34, solvent: [DMSO], spectrometer: 399.95 MHz
8.796 (0.4); 8.783 (0.5); 8.769 (6.7); 8.767 (5.8); 8.757 (6.8); 8.755 (5.7); 8.173 (0.8); 7.428 (1.8); 7.416
(3.3); 7.405 (1.6); 5.512 (0.4); 5.345 (2.9); 5.286 (7.2); 5.264 (6.5); 4.619 (0.8); 3.373 (0.4); 3.168 (1.1);
2.524 (0.7); 2.510 (14.0); 2.506 (27.6); 2.501 (35.7); 2.497 (25.2); 2.492 (11.8); 2.244 (0.6); 2.174
(14.7); 2.074 (1.7); 1.946 (16.0); 1.790 (0.9); 0.000 (7.3)
Compound No. II-1-35: 1H NMR (DMSO-D₆) 4.99 (s, 2H), 5.36 (s, 2H), 5.98 (s, 1H), 7.41-7.44 (t,
1H), 8.75-8.77 (d, 2H).
Compound No. II-1-36: ¹H NMR (400.0 MHz, CD3CN):
8.700 (7.7); 8.688 (7.7); 7.315 (2.1); 7.302 (3.9); 7.290 (1.9); 5.645 (8.4); 5.317 (0.6); 5.281 (16.0); 3.963 (2.1); 3.544
(0.4); 3.526 (0.4); 3.274 (4.5); 2.176 (7.1); 2.114 (0.5); 2.107 (0.5); 2.101 (0.4); 2.080 (0.3); 1.964 (1.7); 1.952 (14.0);
1.946 (24.2); 1.940 (31.2); 1.934 (21.5); 1.928 (11.0); 1.131 (0.4); 1.114 (0.9); 1.096 (0.4); 0.000 (0.7)
Compound No. II-1-37: ¹H NMR (400.0 MHz, d₆-DMSO):
5.404 (2.1); 4.684 (2.1); 3.328 (9.7); 2.505 (5.8); 2.501 (7.6); 2.497 (5.7); 2.324 (8.2); 1.855 (0.5); 1.835 (16.0); 0.006
(0.4); 0.000 (8.4); −0.008 (0.4)
Compound No. II-1-38: ¹H NMR (600.1 MHz, d₆-DMSO):
7.619 (2.8); 7.615 (2.8); 6.010 (2.7); 6.006 (2.7); 5.891 (10.7); 5.258 (4.0); 4.568 (4.5); 3.328 (10.0); 2.504 (2.2); 2.501
(4.6); 2.498 (6.3); 2.495 (4.5); 2.493 (2.1); 2.283 (14.4); 2.120 (16.0)
Compound No. II-1-39: ¹H NMR (400.0 MHz, d₆-DMSO):
7.311 (3.3); 7.308 (3.2); 6.000 (3.2); 5.932 (11.7); 5.241 (4.5); 4.565 (5.2); 4.046 (1.2); 3.326 (16.9); 2.505 (18.6); 2.501
(23.3); 2.496 (17.0); 2.448 (15.9); 2.322 (16.0); 0.000 (8.2); −0.008 (0.4)
Compound No. II-1-40: ¹H NMR (400.0 MHz, d₆-DMSO):
7.242 (1.7); 7.107 (3.5); 6.973 (1.7); 5.696 (3.7); 4.751 (3.7); 3.620 (16.0); 3.331 (17.7); 2.511 (4.1); 2.507 (8.3); 2.502
(11.0); 2.498 (8.0); 2.494 (4.0); 0.008 (0.4); 0.000 (11.1); −0.009 (0.5)
Compound No. II-1-41: ¹H NMR (400.0 MHz, d₆-DMSO):
7.697 (2.8); 7.694 (2.8); 6.908 (3.0); 6.907 (3.0); 6.905 (3.0); 6.167 (0.3); 6.105 (10.5); 5.296 (4.7); 4.635 (5.3); 3.327
(13.0); 2.505 (16.0); 2.501 (20.6); 2.497 (15.7); 2.329 (16.0); 2.297 (0.6); 1.054 (0.4); 0.008 (1.0); 0.000 (19.8);
−0.008 (1.4)
Compound No. II-1-42: ¹H NMR (400.0 MHz, d₆-DMSO):
7.991 (2.5); 7.988 (2.5); 6.730 (2.8); 6.724 (2.8); 6.132 (10.5); 5.307 (4.7); 4.677 (5.4); 3.332 (14.4); 2.506 (13.9); 2.502
(17.9); 2.498 (13.4); 2.291 (16.0); 0.008 (0.8); 0.000 (17.3); −0.008 (0.8)
Compound No. II-1-43: ¹H NMR (400.0 MHz, d₆-DMSO):
8.611 (6.2); 7.973 (5.7); 6.130 (11.5); 5.285 (4.5); 4.654 (5.0); 3.331 (9.1); 2.510 (6.0); 2.506 (11.9); 2.501 (15.4); 2.497
(11.0); 2.493 (5.2); 2.314 (16.0); 0.000 (5.4)
Compound No. II-1-44: ¹H NMR (400.0 MHz, d₆-DMSO):
8.316 (0.7); 5.333 (10.8); 5.296 (4.7); 4.699 (5.3); 3.910 (2.5); 3.892 (4.7); 3.872 (2.9); 3.472 (3.0); 3.453 (4.8); 3.434
(2.5); 3.329 (10.2); 2.506 (11.4); 2.502 (14.7); 2.497 (10.7); 2.180 (16.0); 0.000 (4.6)
Compound No. II-1-45: ¹H NMR (400.0 MHz, d₆-DMSO):
8.317 (0.7); 8.095 (4.5); 8.093 (4.6); 7.722 (4.4); 7.720 (4.3); 6.342 (11.8); 5.309 (4.4); 4.685 (4.7); 3.330 (7.6); 2.510
(6.5); 2.506 (13.0); 2.501 (16.9); 2.497 (12.2); 2.493 (5.9); 2.306 (16.0); 2.142 (0.4); 1.055 (0.5); 0.008 (1.0); 0.000
(23.5); −0.009 (0.9)
Compound No. II-1-46: ¹H NMR (400.0 MHz, d₆-DMSO):
5.525 (8.7); 5.232 (3.3); 5.231 (3.3); 4.539 (4.0); 3.326 (14.3); 3.084 (16.0); 2.510 (7.0); 2.506 (13.8); 2.501 (18.0);
2.497 (13.1); 2.492 (6.3); 2.267 (11.5); 2.121 (15.7); 0.000 (2.7)
Compound No. II-1-47: ¹H NMR (400.0 MHz, d₆-DMSO):
8.316 (0.5); 5.458 (8.0); 5.244 (3.5); 4.556 (4.2); 4.103 (0.9); 4.090 (0.9); 3.875 (1.0); 3.863 (16.0); 3.857 (6.0); 3.327
(29.7); 3.175 (4.3); 3.162 (3.7); 2.691 (0.6); 2.684 (0.7); 2.676 (1.4); 2.667 (0.8); 2.659 (0.8); 2.649 (0.4); 2.644
(0.4); 2.524 (0.4); 2.510 (10.3); 2.506 (20.4); 2.502 (26.8); 2.497 (20.0); 2.260 (11.7); 0.858 (1.5); 0.853 (2.3); 0.849
(1.8); 0.836 (5.5); 0.829 (4.5); 0.821 (4.0); 0.809 (0.4); 0.000 (3.6)
Compound No. II-1-48: ¹H NMR (400.0 MHz, d₆-DMSO):
5.755 (5.0); 5.475 (8.4); 5.272 (3.6); 4.628 (4.1); 3.325 (6.4); 3.103 (16.0); 2.895 (15.4); 2.506 (9.0); 2.502 (11.5); 2.497
(8.3); 2.199 (12.2); 0.000 (5.8)
Compound No. II-1-49: ¹H NMR (400.0 MHz, d₆-DMSO):
8.316 (1.3); 5.756 (0.4); 5.479 (8.6); 5.230 (3.5); 4.542 (4.1); 3.325 (16.4); 3.301 (0.4); 2.778 (0.6); 2.775 (0.6); 2.773
(0.5); 2.771 (0.6); 2.764 (1.3); 2.756 (0.6); 2.752 (0.6); 2.748 (0.6); 2.737 (0.4); 2.510 (6.8); 2.506 (13.6); 2.501
(17.9); 2.497 (12.9); 2.492 (6.2); 2.256 (12.1); 2.175 (16.0); 2.170 (3.9); 0.902 (1.4); 0.897 (2.6); 0.886 (3.5); 0.879
(6.4); 0.865 (1.3); 0.858 (0.4); 0.008 (0.3); 0.000 (9.8); −0.009 (0.3)
Compound No. II-1-50: ¹H NMR (400.0 MHz, d₆-DMSO):
11.156 (0.7); 8.316 (1.5); 5.756 (0.9); 5.500 (0.4); 5.495 (0.5); 5.453 (11.1); 5.225 (4.7); 4.552 (5.8); 3.326 (56.4); 3.302
(0.6); 3.110 (0.8); 2.873 (0.4); 2.862 (0.8); 2.855 (1.0); 2.846 (1.7); 2.836 (0.9); 2.830 (1.0); 2.819 (0.9); 2.811
(0.6); 2.803 (1.1); 2.792 (0.5); 2.786 (0.4); 2.524 (0.6); 2.511 (14.1); 2.506 (28.5); 2.502 (37.7); 2.497 (27.3); 2.493
(13.0); 2.278 (0.6); 2.266 (0.8); 2.261 (0.7); 2.248 (16.0); 1.961 (0.4); 1.948 (1.0); 1.940 (1.0); 1.935 (1.0); 1.928 (2.1);
1.920 (0.8); 1.915 (1.8); 1.907 (1.2); 1.902 (0.7); 1.894 (0.9); 0.956 (2.1); 0.951 (3.2); 0.946 (3.6); 0.934 (8.5); 0.929
(7.1); 0.926 (8.1); 0.922 (7.5); 0.915 (6.5); 0.907 (4.8); 0.898 (1.9); 0.892 (1.7); 0.885 (2.0); 0.877 (0.8); 0.805
(1.3); 0.796 (3.2); 0.790 (4.5); 0.784 (4.4); 0.778 (4.4); 0.772 (1.9); 0.767 (1.0); 0.760 (0.6); 0.008 (0.6); 0.000 (19.8);
−0.009 (0.7)
Compound No. II-1-51: ¹H NMR (400.0 MHz, d₆-DMSO):
8.689 (7.7); 8.677 (7.8); 7.278 (2.2); 7.265 (4.2); 7.253 (2.1); 5.680 (12.9); 5.261 (4.5); 4.628 (5.1); 3.329 (19.2); 2.507
(16.5); 2.502 (21.4); 2.498 (15.7); 2.479 (0.4); 2.200 (16.0); 0.008 (0.6); 0.000 (16.0); −0.009 (0.6)
Compound No. II-1-52: ¹H NMR(601.6 MHz, d₆-DMSO):
5.506 (9.7); 5.274 (2.8); 5.273 (2.9); 4.546 (2.4); 3.342 (2.9); 2.508 (2.4); 2.505 (5.5); 2.502 (7.7); 2.499 (5.7); 2.496
(2.6); 2.219 (0.3); 2.202 (10.3); 2.201 (10.8); 1.789 (0.5); 1.780 (15.4); 1.759 (0.4); 1.742 (16.0); 1.731 (0.4); 0.005
(0.4); 0.000 (13.4); −0.006 (0.4)
Compound No. II-1-53: ¹H NMR (400.0 MHz, d₆-DMSO):
5.235 (4.5); 4.492 (1.8); 4.058 (1.3); 4.040 (1.4); 4.021 (1.8); 4.003 (1.8); 3.836 (1.9); 3.820 (1.9); 3.799 (1.4); 3.783
(1.4); 3.326 (9.3); 2.510 (5.2); 2.506 (10.3); 2.501 (13.4); 2.497 (9.6); 2.493 (4.6); 2.185 (0.4); 2.165 (0.9); 2.158
(0.6); 2.147 (1.1); 2.133 (16.0); 2.123 (1.1); 2.104 (0.5); 1.799 (1.8); 1.780 (2.0); 1.772 (1.6); 1.753 (1.7); 1.501 (1.8);
1.482 (2.8); 1.463 (1.5); 0.000 (6.7)
Compound No. II-1-54: ¹H NMR (400.0 MHz, d₆-DMSO):
5.520 (0.4); 5.480 (8.8); 5.233 (3.4); 4.633 (3.8); 3.327 (17.8); 3.227 (16.0); 3.186 (1.2); 3.175 (1.2); 3.162 (1.1); 2.510
(6.5); 2.506 (13.1); 2.502 (17.3); 2.497 (12.6); 2.493 (6.2); 2.394 (0.9); 2.379 (15.1); 2.312 (0.8); 2.300 (12.1);
2.035 (1.2); 1.055 (0.5); 0.008 (0.8); 0.000 (21.9); −0.009 (0.9)
Compound No. II-1-55: ¹H NMR(601.6 MHz, d₆-DMSO):
8.321 (4.4); 5.641 (0.4); 5.325 (10.1); 5.248 (3.7); 4.480 (2.3); 3.344 (1.8); 2.508 (4.7); 2.505 (10.2); 2.502 (14.0); 2.499
(10.1); 2.497 (4.7); 2.228 (0.5); 2.215 (13.8); 2.076 (0.3); 2.041 (0.7); 1.595 (16.0); 0.005 (0.5); 0.000 (19.3); −0.006
(0.7)
Compound No. II-1-56: ¹H NMR (400.0 MHz, d₆-DMSO):
6.234 (1.7); 6.218 (3.8); 6.201 (1.9); 5.240 (4.4); 5.239 (4.2); 4.521 (8.4); 4.505 (7.8); 3.325 (14.3); 2.510 (8.2); 2.506
(15.4); 2.502 (19.6); 2.497 (14.2); 2.422 (0.4); 2.117 (16.0); 0.008 (1.3); 0.000 (25.1); −0.009 (1.1)
Compound No. II-1-57: ¹H NMR (600.1 MHz, d₆-DMSO):
6.395 (1.5); 5.226 (4.7); 5.186 (11.7); 4.467 (5.1); 3.334 (24.0); 3.309 (2.1); 3.300 (3.1); 3.290 (2.2); 3.173 (0.7); 3.165
(0.7); 3.130 (0.4); 3.074 (1.2); 3.070 (1.4); 3.064 (2.1); 3.061 (2.1); 3.055 (1.5); 3.051 (1.2); 2.507 (2.4); 2.504
(5.3); 2.501 (7.3); 2.498 (5.3); 2.495 (2.4); 2.182 (16.0); 2.181 (15.5); 1.753 (0.6); 1.743 (1.5); 1.734 (2.3); 1.724 (1.4);
1.714 (0.5)
Compound No. II-1-58: ¹H NMR (400.0 MHz, d₆-DMSO):
7.445 (1.0); 7.441 (0.9); 7.435 (1.6); 7.427 (1.7); 7.421 (1.3); 7.416 (0.6); 7.309 (2.1); 7.302 (8.3); 7.298 (3.8); 7.294
(5.5); 7.279 (1.4); 5.288 (4.5); 4.830 (10.7); 4.478 (4.0); 3.871 (10.4); 3.329 (107.8); 3.174 (1.5); 3.162 (1.5); 2.675
(0.3); 2.671 (0.5); 2.666 (0.4); 2.524 (0.9); 2.519 (1.4); 2.511 (26.8); 2.506 (56.7); 2.502 (77.0); 2.497 (57.4); 2.493
(28.9); 2.333 (0.4); 2.329 (0.5); 2.324 (0.4); 2.138 (0.4); 2.099 (0.6); 2.042 (0.7); 2.028 (16.0); 1.397 (0.5); 0.000
(0.4)
Compound No. II-1-59: ¹H NMR (400.0 MHz, d₆-DMSO):
5.188 (4.9); 5.187 (4.7); 5.181 (0.7); 5.177 (1.1); 5.174 (1.3); 5.170 (1.0); 5.167 (0.5); 5.164 (0.4); 5.160 (0.6); 5.157
(0.7); 5.154 (0.5); 4.351 (4.4); 4.333 (3.1); 3.328 (8.6); 2.510 (4.5); 2.506 (9.0); 2.501 (11.7); 2.497 (8.4); 2.492 (3.9);
2.072 (16.0); 2.070 (15.0); 1.693 (9.7); 1.673 (8.6); 1.671 (8.4); 0.008 (0.7); 0.000 (19.0); −0.009 (0.6)
Compound No. II-1-60: ¹H NMR (400.0 MHz, d₆-DMSO):
5.288 (3.1); 4.913 (9.4); 4.516 (1.8); 3.329 (8.6); 2.510 (3.2); 2.506 (6.4); 2.501 (8.5); 2.497 (6.2); 2.493 (3.1); 2.139
(11.4); 2.138 (11.5); 2.099 (16.0); 0.000 (6.7)
Compound No. II-1-61: ¹H NMR (400.0 MHz, d₆-DMSO):
5.285 (4.4); 4.952 (11.5); 4.509 (5.1); 3.326 (15.5); 2.616 (1.5); 2.597 (4.6); 2.579 (4.8); 2.560 (1.7); 2.502 (18.6); 2.155
(0.6); 2.140 (16.0); 1.178 (4.9); 1.160 (9.6); 1.142 (4.8); 0.000 (9.9)
Compound No. II-1-62: ¹H NMR (400.0 MHz, d₆-DMSO):
8.859 (1.8); 8.847 (1.8); 8.807 (0.7); 8.801 (10.9); 8.788 (11.2); 8.781 (0.7); 8.773 (1.2); 8.768 (0.5); 7.505 (0.5); 7.493
(1.0); 7.480 (0.7); 7.475 (2.6); 7.463 (4.9); 7.451 (2.5); 7.446 (0.7); 6.312 (0.4); 6.223 (10.8); 5.861 (1.0); 5.504
(0.5); 5.426 (16.0); 5.409 (0.6); 4.808 (4.3); 2.525 (0.4); 2.520 (0.7); 2.511 (13.9); 2.507 (29.0); 2.502 (38.7); 2.498
(28.7); 2.493 (14.5); 0.008 (1.1); 0.000 (40.3); −0.009 (1.8)
Compound No. II-1-63: ¹H NMR (400.0 MHz, d₆-DMSO):
7.821 (4.3); 5.248 (3.8); 5.165 (9.1); 4.505 (4.1); 3.828 (16.0); 3.328 (6.1); 2.505 (8.7); 2.501 (11.4); 2.497 (9.0); 2.196
(12.9); 0.008 (0.9); 0.000 (19.4)
Compound No. II-1-64: ¹H NMR (400.0 MHz, d₆-DMSO):
5.755 (0.4); 5.512 (0.8); 5.495 (2.7); 5.478 (2.7); 5.460 (0.8); 5.308 (4.3); 4.758 (4.2); 3.325 (8.5); 2.519 (0.3); 2.510
(5.1); 2.506 (10.4); 2.502 (13.7); 2.497 (9.7); 2.492 (4.6); 2.142 (16.0); 1.629 (10.2); 1.611 (10.0); 0.008 (0.6); 0.000
(17.4); −0.009 (0.6)

The compounds for the formulae (I-1), (I-2), (I-3) and (I-4) described in Tables 1 to 4 are likewise preferred compounds which were obtained according to or analogously to the synthesis examples described above. In the tables, Me represents methyl and Et represents ethyl.

Compounds of the formula (I-1)

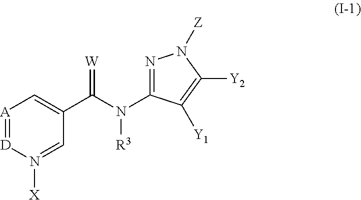

in which X, unless stated otherwise, is a free electron pair, and A, D, W, R³, Y₁, Y₂ and Z are each as defined in Table 1.

TABLE 1

| Compd. | | | | | | | | | log P |
|---|---|---|---|---|---|---|---|---|---|
| No. | X | D | A | R³ | W | Y₁ | Y₂ | Z | (acid)¹ |
| I-1-1 | | C—H | C—H | H | O | H | methyl | pyrimidin-2-ylmethyl | 0.77 |
| I-1-10 | | C—H | C—H | H | O | H | H | 2,2,2-trifluoroethyl | 1.43 |
| I-1-100 | | C—Cl | C—Cl | H | O | H | methyl | benzyl | 3.18 |
| I-1-101 | | C—H | C—Br | H | O | H | methyl | 2-fluorobenzyl | 2.69 |
| I-1-102 | | C—Me | C—H | H | O | H | methyl | 2-fluorobenzyl | 1.73 |
| I-1-103 | | C—Cl | C—Cl | H | O | H | methyl | 2-fluorobenzyl | 3.28 |
| I-1-104 | | C—OMe | C—H | H | O | H | methyl | benzyl | 2.43 |
| I-1-105 | | C—OMe | C—H | H | O | H | methyl | 2-fluorobenzyl | 2.52 |
| I-1-106 | | C—Cl | C—H | H | O | H | methyl | 3-fluorobenzyl | 2.6 |
| I-1-107 | | C—H | C—Br | H | O | H | methyl | 3-fluorobenzyl | 2.71 |
| I-1-108 | | C—Me | C—H | H | O | H | methyl | 3-fluorobenzyl | 1.79 |
| I-1-109 | | C—Me | C—H | H | O | H | methyl | 4-fluorobenzyl | 1.76 |
| I-1-11 | | C—H | C—H | H | O | H | methyl | cyanomethyl | 0.7 |
| I-1-110 | | C—OMe | C—H | H | O | H | methyl | 4-fluorobenzyl | 2.47 |
| I-1-111 | | C—H | C—H | H | O | H | difluoro- | methyl | 0.89 |
| | | | | | | | methoxy | | |
| I-1-112 | | C—H | C—F | H | O | H | difluoro- | methyl | 1.56 |
| | | | | | | | methoxy | | |
| I-1-113 | | C—H | C—F | H | O | bro- | difluoro- | methyl | 1.67 |
| | | | | | | mine | methoxy | | |
| I-1-114 | | C—H | C—F | H | O | H | methyl | carboxymethyl | 0.87 |
| I-1-115 | | C—H | C—H | H | O | H | methyl | carboxymethyl | 0.09 |
| I-1-116 | | C—H | C—H | H | O | H | H | [3-chloro-5- | 1.86 |
| | | | | | | | | (trifluoromethyl)pyridin-2- | |
| | | | | | | | | yl]methyl | |
| I-1-117 | | C—H | C—H | H | O | H | H | 2-cyanophenyl | 1.42 |
| I-1-118 | | C—Cl | C—H | H | O | H | H | [3-chloro-5- | 2.64 |
| | | | | | | | | (trifluoromethyl)pyridin-2- | |
| | | | | | | | | yl]methyl | |
| I-1-119 | | C—H | C—H | H | O | 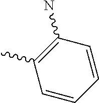 | | methyl | 1.13 |
| I-1-12 | | C—H | C—H | H | O | H | methyl | isobutyl | 1.59 |
| I-1-120 | | C—H | C—H | H | O | H | methyl | acetyl | 1.18 |
| I-1-121 | | C—H | C—H | H | O | H | methyl | cyclopropylcarbonyl | 1.65 |
| I-1-122 | | C—H | C—H | H | O | H | methyl | 2-chlorobenzoyl | 2.27 |
| I-1-123 | | C—H | C—H | H | O | methyl | methyl | pyrimidin-2-ylmethyl | 0.76 |
| I-1-124 | | C—H | C—H | H | O | H | methyl | 2-(methylamino)-2- | 0.29 |
| | | | | | | | | oxoethyl | |
| I-1-125 | | C—H | C—H | H | O | bro- | methyl | pyrimidin-2-ylmethyl | 0.98 |
| | | | | | | mine | | | |
| I-1-126 | | C—H | C—H | H | O | H | methyl | methoxycarbonyl | 1.08 |
| I-1-127 | | C—H | C—H | H | O | H | methyl | 3-chlorobenzoyl | 1.92 |
| I-1-128 | O | C—H | C—H | H | O | H | methyl | pyrimidin-2-ylmethyl | 0.55 |
| I-1-129 | | C—H | C—H | H | O | H | methyl | propionyl | 1.56 |
| I-1-13 | | C—H | C—H | H | O | H | H | 2,2-difluoroethyl | 0.74 |
| I-1-130 | | C—H | C—H | H | O | H | methyl | isobutyryl | 1.95 |
| I-1-131 | | C—H | C—H | H | O | H | methyl | butyryl | 1.94 |
| I-1-132 | | C—H | C—F | H | O | H | methyl | 4,6-dimethylpyrimidin-2-yl | 2.04 |
| I-1-133 | | C—H | C—H | H | O | H | methyl | 2,2,2-trifluoroethyl | 1.32 |
| I-1-134 | | C—H | C—F | H | O | H | methoxy | methyl | 1.5 |
| | | | | | | | carbonyl | | |
| I-1-135 | | C—H | C—F | H | O | H | methyl | 2,2,2-trifluoroethyl | 1.79 |
| I-1-136 | | C—H | C—H | H | O | methyl | H | pyrimidin-2-ylmethyl | 0.44 |
| I-1-137 | | C—H | C—H | H | O | chlo- | H | pyrimidin-2-ylmethyl | 0.58 |
| | | | | | | rine | | | |
| I-1-138 | | C—H | C—H | H | O | H | methoxy | methyl | 1.05 |
| | | | | | | | carbonyl | | |
| I-1-139 | | C—H | C—H | H | O | H | methyl | 4,6-dimethylpyrimidin-2-yl | 1.6 |
| I-1-14 | | C—H | C—H | H | O | H | methyl | 1,3-thiazol-2-ylmethyl | 1.05 |
| I-1-140 | | C—H | C—H | H | O | H | bromine | pyrimidin-2-ylmethyl | 1.1 |
| I-1-15 | | C—H | C—H | H | O | H | methyl | pyridin-3-ylmethyl | 0.12 |
| I-1-16 | | C—H | C—H | methyl | O | H | methyl | methyl | 0.65 |
| I-1-17 | | C—H | C—H | H | O | H | methyl | H | 0.3 |
| I-1-18 | | C—H | C—H | H | O | H | methyl | tert-butoxycarbonyl | 2.04 |
| I-1-19 | | C—H | C—H | H | O | H | methyl | prop-2-yn-1-yl | 0.93 |
| I-1-2 | | C—H | C—H | H | O | H | methyl | ethoxymethyl | 1.1 |
| I-1-20 | | C—H | C—H | H | O | H | methyl | (3-cyanopyridin-2- | 1.08 |
| | | | | | | | | yl)methyl | |
| I-1-21 | | C—H | C—H | H | O | H | methyl | (6-chloropyridin-3- | 1.42 |
| | | | | | | | | yl)methyl | |
| I-1-22 | | C—H | C—H | 3- | O | H | methyl | 3-chlorobenzoyl | 3.98 |
| | | | | chloro- | | | | | |
| | | | | benzoyl | | | | | |
| I-1-23 | | C—H | C—H | H | O | H | methyl | pyrimidin-2-yl | 1.16 |
| I-1-24 | | C—H | C—H | H | O | H | methyl | 2-ethoxy-2-oxoethyl | 0.89 |
| I-1-25 | | C—H | C—H | methyl | O | H | methyl | pyrimidin-2-yl | 1.02 |
| I-1-26 | | C—H | C—H | H | O | H | H | pyridin-2-ylmethyl | 0.57 |
| I-1-27 | | C—H | C—H | H | O | H | H | (6-chloropyridin-3- | 1.22 |
| | | | | | | | | yl)methyl | |
| I-1-28 | | C—H | C—H | H | O | H | methyl | pyridin-3-ylcarbonyl | 1.57 |
| I-1-29 | | C—H | C—H | H | O | H | cyclo- | (6-chloropyridin-3- | 1.77 |
| | | | | | | | propyl | yl)methyl | |
| I-1-3 | | C—H | C—H | H | O | H | methyl | methyl | 0.63 |
| I-1-30 | | C—H | C—H | H | O | H | H | 3-fluoropyridin-2-yl | 1.15 |
| I-1-31 | | C—H | C—H | H | O | H | methyl | (4-methylpyrimidin-2- | 0.96 |
| | | | | | | | | yl)methyl | |
| I-1-32 | | C—H | C—H | H | O | H | methyl | 2-[(1-methoxy-2-methyl-1- | 0.72 |
| | | | | | | | | oxopropan-2-yl)amino]-2- | |
| | | | | | | | | oxoethyl | |
| I-1-33 | | C—H | C—H | H | O | H | methyl | 2-chloroethyl | 1.16 |
| I-1-34 | | C—H | C—H | H | O | H | methyl | 3-chloropyridin-2-yl | 1.35 |
| I-1-35 | | C—H | C—H | H | O | H | ethyl | benzyl | 2.03 |
| I-1-36 | | C—H | C—H | H | O | H | methyl | (4,6-dimethylpyrimidin-2- | 1.2 |
| | | | | | | | | yl)methyl | |
| I-1-37 | | C—H | C—H | H | O | H | methyl | (3-chloropyridin-2- | 1.42 |
| | | | | | | | | yl)methyl | |
| I-1-38 | | C—H | C—H | H | O | H | methyl | ethyl | 0.93 |
| I-1-39 | | C—H | C—H | H | O | 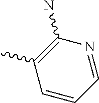 | | methyl | 0.78 |
| I-1-4 | | C—H | C—H | H | O | H | methyl | 2,2-difluoroethyl | 1.05 |
| I-1-40 | | C—H | C—H | H | O | H | ethyl | prop-2-yn-1-yl | 1.31 |
| I-1-41 | | C—H | C—F | H | O | H | methyl | 2-[(1-methoxy-2-methyl-1- | 1.33 |
| | | | | | | | | oxopropan-2-yl)amino]-2- | |
| | | | | | | | | oxoethyl | |
| I-1-42 | | C—H | C—H | 4- | O | H | methyl | 4-chlorobenzoyl | 4.18 |
| | | | | chloro- | | | | | |
| | | | | benzoyl | | | | | |
| I-1-43 | | C—H | C—H | H | O | H | cyclo- | prop-2-yn-1-yl | 1.41 |
| | | | | | | | propyl | | |
| I-1-44 | | C—H | C—H | H | O | H | methyl | 3-chloro-5- | 2.2 |
| | | | | | | | | (trifluoromethyl)pyridin-2- | |
| | | | | | | | | yl | |
| I-1-45 | | C—H | C—H | H | O | H | methyl | 5-(trifluoromethyl)pyridin- | 2.64 |
| | | | | | | | | 2-yl | |
| I-1-46 | | C—H | C—H | H | O | H | methyl | benzyl | 1.79 |
| I-1-47 | | C—H | C—H | H | O | bro- | difluoro- | methyl | 1.13 |
| | | | | | | mine | methoxy | | |
| I-1-48 | | C—H | C—H | H | O | H | H | 3-chloropyridin-2-yl | 1.25 |
| I-1-49 | | C—H | C—H | H | O | H | methyl | isopropyl | 1.4 |
| I-1-5 | | C—H | C—H | H | O | H | methyl | 2-fluoroethyl | 0.84 |
| I-1-50 | | C—H | C—H | H | O | H | methyl | 3-methylbenzyl | |
| I-1-51 | | C—H | C—H | H | O | H | methyl | pyridin-4-ylmethyl | 0.13 |
| I-1-52 | | C—H | C—H | H | O | H | H | pyridin-2-yl | 1.32 |
| I-1-53 | | C—H | C—H | H | O | H | methyl | (4,6-dimethoxypyrimidin- | 1.59 |
| | | | | | | | | 2-yl)methyl | |
| I-1-54 | | C—H | C—H | H | O | H | H | prop-2-yn-1-yl | 0.69 |
| I-1-55 | | C—H | C—H | H | O | H | methyl | pyrazin-2-yl | 1.27 |
| I-1-56 | | C—H | C—H | H | O | H | cyclo- | benzyl | 2.1 |
| | | | | | | | propyl | | |
| I-1-57 | | C—H | C—F | H | O | 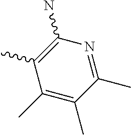 | | methyl | 1.6 |
| I-1-58 | | C—H | C—H | H | O | H | ethyl | (6-chloropyridin-3- | 1.67 |
| | | | | | | | | yl)methyl | |
| I-1-59 | | C—H | C—F | H | O | H | methyl | 2-ethoxy-2-oxoethyl | 1.53 |
| I-1-6 | | C—H | C—H | H | O | H | methyl | pyridin-2-ylmethyl | 0.88 |
| I-1-60 | | C—H | C—H | H | O | H | methyl | 3-fluorobenzyl | 1.9 |
| I-1-61 | | C—H | C—F | H | O | H | methyl | 2-[(2-ethoxy-2- | 1.35 |
| | | | | | | | | oxoethyl)(methyl)amino]- | |
| | | | | | | | | 2-oxoethyl | |
| I-1-62 | | C—H | C—H | H | O | H | methyl | 4-fluorobenzyl | 1.88 |
| I-1-63 | | C—H | C—H | H | O | H | H | 3-chloro-5- | 2.18 |
| | | | | | | | | (trifluoromethyl)pyridin-2- | |
| | | | | | | | | yl | |
| I-1-64 | | C—H | C—H | H | O | H | methyl | 2-fluorobenzyl | 1.82 |
| I-1-65 | | C—H | C—H | H | O | H | H | 1,3-difluoropropan-2-yl | 0.84 |
| I-1-66 | | C—H | N | H | O | H | methyl | benzyl | 1.76 |
| I-1-67 | | C—H | C—H | H | O | H | methyl | 4-methoxy-4-oxobutyl | |
| I-1-68 | | C—H | C—H | H | O | H | H | 2,6-difluorobenzyl | 1.55 |
| I-1-69 | | C—H | C—H | H | O | H | H | 2-ethoxy-2-oxoethyl | |
| I-1-7 | | C—H | C—F | H | O | H | methyl | pyrimidin-2-ylmethyl | 1.11 |
| I-1-70 | | C—H | C—H | H | O | H | H | 3-fluorobenzyl | 1.59 |
| I-1-71 | | C—H | C—H | H | O | H | H | 3-(trifluoromethyl)benzyl | 2.07 |
| I-1-72 | | C—H | C—H | H | O | H | H | 3-methylbenzyl | 1.83 |
| I-1-73 | | C—H | C—H | H | O | H | H | 3-(trifluoromethoxy)benzyl | 2.20 |
| I-1-74 | | C—H | C—H | H | O | H | H | 3-methoxybenzyl | 1.57 |
| I-1-75 | | C—H | C—H | H | O | H | H | 4-tert-butylbenzyl | 2.68 |
| I-1-76 | | C—H | C—H | H | O | H | H | 2,4-difluorobenzyl | 1.68 |
| I-1-77 | | C—H | C—H | H | O | H | H | 3- | 1.57 |
| | | | | | | | | (methoxycarbonyl)benzyl | |
| I-1-78 | | C—H | C—H | H | O | H | H | 4-methoxybenzyl | 1.55 |
| I-1-79 | | N | C—H | H | O | H | methyl | benzyl | 1.67 |
| I-1-8 | | C—H | C—H | H | O | H | methyl | difluoromethyl | 1.19 |
| I-1-80 | | C—Me | C—H | H | O | H | methyl | benzyl | 1.71 |
| I-1-81 | | C—H | C—H | H | O | H | H | 2,5-dimethylbenzyl | 2.11 |
| I-1-82 | | C—H | C—H | H | O | H | H | 2,4-dichlorobenzyl | |
| I-1-83 | | C—H | C—Br | H | O | H | methyl | 4-fluorobenzyl | 2.67 |
| I-1-84 | | C—H | C—H | H | O | H | H | benzyl | 1.52 |
| I-1-85 | | C—H | C—H | H | O | H | H | 2,4-difluorophenyl | |
| I-1-86 | | C—Cl | C—H | H | O | H | H | 4-tert-butylbenzyl | 3.54 |
| I-1-87 | | C—Cl | C—H | H | O | H | H | 3-(trifluoromethyl)benzyl | 2.82 |
| I-1-88 | | C—H | C—H | H | O | H | H | 2-fluorobenzyl | 1.58 |
| I-1-89 | | C—H | C—H | H | O | H | H | 2-cyanobenzyl | 1.37 |
| I-1-9 | | C—H | C—H | H | O | H | H | pyrimidin-2-ylmethyl | 0.42 |
| I-1-90 | | C—H | C—H | H | O | H | H | 2,5-difluorobenzyl | 1.65 |
| I-1-91 | | C—H | C—H | H | O | H | H | 2-(trifluoromethyl)benzyl | 2.09 |
| I-1-92 | | C—H | C—H | H | O | H | H | 4-(ethoxycarbonyl)benzyl | 1.85 |
| I-1-93 | | C—Cl | C—H | H | O | H | methyl | 2-fluorobenzyl | 2.59 |
| I-1-94 | | C—OMe | C—H | H | O | H | methyl | 3-fluorobenzyl | 2.51 |
| I-1-95 | | C—Cl | C—H | H | O | H | methyl | 4-fluorobenzyl | 2.57 |
| I-1-96 | | C—Cl | C—Cl | H | O | H | methyl | 3-fluorobenzyl | 3.26 |
| I-1-97 | | C—Cl | C—Cl | H | O | H | methyl | 4-fluorobenzyl | 3.23 |
| I-1-98 | | C—Cl | C—H | H | O | H | methyl | benzyl | 2.48 |
| I-1-99 | | C—H | C—Br | H | O | H | methyl | benzyl | 2.58 |
| I-1-141 | | C—H | C—H | H | O | CN | SOMe | Me | 0.06 |
| I-1-142 | | C—H | C—H | H | O | H | Me | [(2E)-2-(cyanoimino)-1,3- | 1.08 |
| | | | | | | | | thiazolidin-3-yl]methyl | |
| I-1-143 | | C—H | C—H | H | O | H | Me | (3,4-dimethyl-5-oxo-4,5- | 0.72 |
| | | | | | | | | dihydro-1H-1,2,4-triazol-1- | |
| | | | | | | | | yl)methyl | |
| I-1-144 | | C—H | C—H | H | O | H | Me | (4-cyclopropyl-3-methoxy- | 1.27 |
| | | | | | | | | 5-oxo-4,5-dihydro-1H- | |
| | | | | | | | | 1,2,4-triazol-1-yl)methyl | |
| I-1-145 | | C—H | C—H | H | O | H | Me | (2,4-dimethyl-3,5-dioxo- | 0.82 |
| | | | | | | | | 1,2,4-triazolidin-1- | |
| | | | | | | | | yl)methyl | |
| I-1-146 | | C—H | C—H | H | O | H | Me | (4-cyclopropyl-3-methyl-5- | 1.05 |
| | | | | | | | | oxo-4,5-dihydro-1H-1,2,4- | |
| | | | | | | | | triazol-1-yl)methyl | |
| I-1-147 | | C—H | C—H | H | O | H | Me | (3,4-dicyclopropyl-5-oxo- | 1.45 |
| | | | | | | | | 4,5-dihydro-1H-1,2,4- | |
| | | | | | | | | triazol-1-yl)methyl | |
| I-1-148 | | C—H | C—H | H | O | H | Me | (1,3-dimethyl-5-oxo-1,5- | 0.74 |
| | | | | | | | | dihydro-4H-1,2,4-triazol-4- | |
| | | | | | | | | yl)methyl | |
| I-1-149 | | C—H | C—H | H | O | H | Me | (2-oxotetrahydropyrimidin- | 0.62 |
| | | | | | | | | 1(2H)-yl)methyl | |
| I-1-150 | | N | C—H | H | O | H | Me | (3,4-dicyclopropyl-5-oxo- | 1.33 |
| | | | | | | | | 4,5-dihydro-1H-1,2,4- | |
| | | | | | | | | triazol-1-yl)methyl | |
| I-1-151 | | N | C—H | H | O | H | Me | (4-cyclopropyl-3-methyl-5 | 0.92 |
| | | | | | | | | oxo-4,5-dihydro-1H-1,2,4- | |
| | | | | | | | | triazol-1-yl)methyl | |
| I-1-152 | | N | C—H | H | O | H | Me | (4-cyclopropyl-3-methoxy- | 1.17 |
| | | | | | | | | 5-oxo-4,5-dihydro-1H- | |
| | | | | | | | | 1,2,4-triazol-1-yl)methyl | |
| I-1-153 | | C—H | C—H | H | O | H | Me | (1,4-dimethyl-5-oxo-4,5- | 0.62 |
| | | | | | | | | dihydro-1H-1,2,4-triazol-3- | |
| | | | | | | | | yl)methyl | |
| I-1-154 | | C—H | C—H | H | O | H | Me | (4-methyl-2-oxo-2H-pyran- | |
| | | | | | | | | 6-yl)methyl | |
| I-1-155 | | C—H | C—H | H | O | H | Me | [6-(trifluoromethyl)- | 1.54 |
| | | | | | | | | imidazo[1,2-a]pyridin-2- | |
| | | | | | | | | yl]methyl | |
| I-1-156 | | C—H | C—H | H | O | H | Me | [5-carboxyl-1-(3- | 1.18 |
| | | | | | | | | chlorpyridin-2-yl)-1H- | |
| | | | | | | | | pyrazol-3-yl]methyl | |
| I-1-157 | | C—H | C—H | H | O | H | Me | 2,3-dihydro-1,4- | 1.99 |
| | | | | | | | | benzodioxin-2-ylmethyl | |
| I-1-158 | | C—H | C—H | H | O | H | Me | 5,6,7,8- | 2.73 |
| | | | | | | | | tetrahydronaphthalen-2- | |
| | | | | | | | | ylmethyl | |
| I-1-159 | | C—H | C—H | H | O | H | Me | (6-fluoroimidazo[1,2-a]- | 0.66 |
| | | | | | | | | pyridin-2-yl)methyl | |
| I-1-160 | | C—H | C—H | H | O | H | Me | (6-chloroimidazo[1,2-a]- | 1.03 |
| | | | | | | | | pyridin-2-yl)methyl | |
| I-1-161 | | C—H | C—H | H | O | H | Me | pyrrolidin-1-ylcarbonyl | 1.29 |
| I-1-162 | | C—H | C—H | H | O | H | Me | (4-methylpiperidin-1-yl)- | 2.51 |
| | | | | | | | | sulphonyl | |
| I-1-163 | | C—H | C—H | H | O | H | Me | (2-methoxyethyl)- | 1.22 |
| | | | | | | | | carbamoyl | |
| I-1-164 | | C—H | C—H | H | O | H | Me | [(2,2-dimethyl-1,3- | 1.56 |
| | | | | | | | | dioxolan-4- | |
| | | | | | | | | yl)methyl]carbamoyl | |
| I-1-165 | | C—H | C—H | H | O | H | Me | [3-(methylsulphanyl)- | 1.7 |
| | | | | | | | | propyl]carbamoyl | |
| I-1-166 | | C—H | C—H | H | O | H | Me | (1-methoxypropan-2-yl)- | 1.44 |
| | | | | | | | | carbamoyl | |
| I-1-167 | | C—H | C—H | H | O | H | Me | [2-(methylsulphanyl)- | 1.55 |
| | | | | | | | | ethyl]carbamoyl | |
| I-1-168 | | C—H | C—H | H | O | H | Me | (2- | 2.18 |
| | | | | | | | | phenoxyethyl)carbamoyl | |
| I-1-169 | | C—H | C—H | H | O | H | Me | bis(2- | 1.91 |
| | | | | | | | | methoxyethyl)carbamoyl | |
| I-1-170 | | C—H | C—H | H | O | H | Me | (2,2- | 1.88 |
| | | | | | | | | diethoxyethyl)carbamoyl | |
| I-1-171 | | C—H | C—H | H | O | H | Me | [2-(2,6-difluorophenoxy)- | 2.21 |
| | | | | | | | | ethyl]carbamoyl | |
| I-1-172 | | C—H | C—H | H | O | H | Me | [2-(trifluoromethoxy)- | 1.91 |
| | | | | | | | | ethyl]carbamoyl | |
| I-1-173 | | C—H | C—H | H | O | H | Me | 2-methoxyethyl | 0.79 |
| I-1-174 | | C—H | C—H | H | O | H | Me | cyclopropylmethyl | 1.31 |
| I-1-175 | | C—H | C—H | H | O | H | Me | CH₂OCH₃ | 0.71 |
| I-1-176 | | C—H | C—H | H | O | H | Me | tetrahydrofuran-2-ylmethyl | 1.06 |
| I-1-177 | | C—H | C—H | Me | O | H | Me | cyclopropylmethyl | 1.27 |
| I-1-178 | | C—H | C—H | Me | O | H | Me | CH₂OCH₃ | 0.7 |
| I-1-179 | | C—H | C—H | Me | O | H | Me | 2-methoxyethyl | 0.79 |
| I-1-180 | | C—H | C—H | H | O | H | Me | 1,3-dioxolan-2-ylmethyl | 0.81 |
| I-1-181 | | C—H | C—H | Me | O | H | Me | tetrahydrofuran-2-ylmethyl | 1.05 |
| I-1-182 | | C—H | C—H | H | O | H | Me | 2-(methylsulphonyl)ethyl | 0.49 |
| I-1-183 | | C—H | C—H | H | O | H | Me | 2-(methylsulphinyl)ethyl | 0.25 |
| I-1-184 | | C—H | C—H | H | O | H | Me | 2-(methylsulphanyl)ethyl | 1.22 |
| I-1-185 | | C—H | C—H | H | O | H | Me | 2-cyanoethyl | 0.57 |
| I-1-186 | | C—H | C—H | Me | O | H | Me | 2-(methylsulphinyl)ethyl | 0.16 |
| I-1-187 | | C—H | C—H | Me | O | H | Me | 2-(methylsulphanyl)ethyl | 1.17 |
| I-1-188 | | C—H | C—H | H | O | H | Me | 3,3,3-trifluoropropyl | 1.45 |
| I-1-189 | | C—H | C—H | H | O | H | Me | 2-(ethylsulphanyl)ethyl | 1.53 |
| I-1-190 | | C—H | C—H | H | O | H | Me | 2-(ethylsulphinyl)ethyl | 0.46 |
| I-1-191 | | C—H | C—H | H | O | H | Me | 2-[(methoxycarbonyl)- | 0.98 |
| | | | | | | | | oxy]ethyl | |
| I-1-192 | | C—H | C—H | H | O | H | Me | (3-methyl-1H-pyrazol-1- | 1.16 |
| | | | | | | | | yl)methyl | |
| I-1-193 | | C—H | C—H | H | O | H | Me | 4-methylphenyl | 2.02 |
| I-1-194 | | C—H | C—H | H | O | CN | Me | pyrimidin-2-ylmethyl | 0.63 |
| I-1-195 | | C—H | C—H | H | O | H | Me | (5-chloropyrimidin-2- | 1.23 |
| | | | | | | | | yl)methyl | |
| I-1-196 | | C—H | C—H | H | O | H | CF₃ | pyrimidin-2-ylmethyl | 1.46 |
| I-1-197 | | C—H | C—H | H | O | H | Cl | pyrimidin-2-ylmethyl | 1.04 |
| I-1-198 | | C—H | C—H | H | O | H | H | CH₃ | 0.22 |
| I-1-199 | | C—H | C—H | H | O | Me | H | CH₂CN | 0.37 |
| I-1-200 | | C—H | C—H | H | O | H | Me | 4-methoxyphenyl | 1.34 |
| I-1-201 | | C—H | C—H | H | O | Cl | SMe | CH₃ | 1.25 |
| I-1-202 | | C—H | C—H | H | O | H | Me | 3-fluorophenyl | 1.85 |
| I-1-203 | | C—H | C—H | H | O | H | Me | 4-fluorophenyl | 1.79 |
| I-1-204 | | C—H | C—H | H | O | H | Me | cyclohexyl | 2.02 |
| I-1-205 | | C—H | C—H | H | O | H | Me | s-C₄H₉ | 1.59 |
| I-1-206 | | C—H | C—H | H | O | H | Me | 3,4-dichlorophenyl | 2.59 |
| I-1-207 | | C—H | C—H | H | O | H | Me | 2,4-dichlorophenyl | 2.36 |
| I-1-208 | | C—H | C—H | H | O | H | Me | 3,5-dimethylphenyl | 2.4 |
| I-1-209 | | C—H | C—H | H | O | H | Me | 4-chlorophenyl | 2.09 |
| I-1-210 | | C—H | C—H | H | O | H | Me | pentan-3-yl | 1.92 |
| I-1-211 | | C—H | C—H | H | O | F | Me | pyrimidin-2-ylmethyl | 0.72 |
| I-1-212 | | C—H | C—H | H | O | Cl | Me | pyrimidin-2-ylmethyl | 0.94 |
| I-1-213 | | C—H | C—H | H | O | H | Me | 2-chlorophenyl | 1.86 |
| I-1-214 | | C—H | C—H | H | O | H | Me | 1-cyanoethyl | 0.92 |
| I-1-215 | | C—H | C—H | H | O | 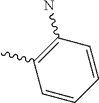 | | pyrimidin-2-ylmethyl | 1.16 |
| I-1-216 | | C—H | C—F | H | O | 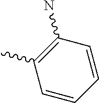 | | pyrimidin-2-ylmethyl | 1.54 |
| I-1-217 | | C—H | C—H | H | O | H | Me | 3-cyanophenyl | 1.59 |
| I-1-218 | | C—H | C—H | H | O | H | Me | 1-cyclopropylethyl | 1.63 |
| I-1-219 | | C—H | C—H | H | O | H | Me | 2-cyanopropan-2-yl | 1.31 |
| I-1-220 | | C—H | C—H | H | O | H | Me | (5-methyl-1H-pyrazol-1- | 1.17 |
| | | | | | | | | yl)methyl | |
| I-1-221 | | C—H | C—H | H | O | H | CHF₂ | CH₃ | 1.04 |
| I-1-222 | | C—H | C—F | H | O | H | CHF₂ | CH₃ | 1.51 |
| I-1-223 | | C—H | C—H | H | O | H | Me | 1H-1,2,3-triazol-1- | 0.65 |
| | | | | | | | | ylmethyl | |
| I-1-224 | | C—H | C—H | H | O | H | Me | 1H-1,2,4-triazol-1- | 0.58 |
| | | | | | | | | ylmethyl | |
| I-1-225 | | C—H | C—H | H | O | H | Me | 1H-pyrazol-1-ylmethyl | 0.94 |
| I-1-226 | | C—H | C—H | H | O | H | Me | (4-methyl-1H-pyrazol-1- | 1.24 |
| | | | | | | | | yl)methyl | |
| I-1-227 | | C—H | C—H | H | O | H | Me | [5-(trifluoromethyl)-1H- | 1.72 |
| | | | | | | | | pyrazol-1-yl]methyl | |
| I-1-228 | | C—H | C—H | H | O | H | Me | [3-(trifluoromethyl)-1H- | 1.86 |
| | | | | | | | | pyrazol-1-yl]methyl | |
| I-1-229 | | C—H | C—H | H | O | H | Me | cyclopentyl | 1.74 |
| I-1-230 | | C—H | C—H | H | O | H | Me | 2-pyridyl | 2.19 |
| I-1-231 | | C—H | C—H | H | O | H | Me | SO₂N(CH₃)₂ | 1.52 |
| I-1-232 | | C—H | C—H | H | (2,4-di- | H | Me | CH₃ | 1.77 |
| | | | | | chloro- | | | | |
| | | | | | phenyl) | | | | |
| | | | | | imino | | | | |
| I-1-233 | | C—H | C—H | H | O | H | Me | CO₂phenyl | 2 |
| I-1-234 | | C—H | C—H | H | O | H | Me | (pyrimidin-2-ylsulphanyl) | 1.25 |
| | | | | | | | | methyl | |
| I-1-235 | | C—H | C—H | H | O | H | Me | (pyrimidin-2-ylsulphinyl) | 0.5 |
| | | | | | | | | methyl | |
| I-1-236 | | C—H | C—H | H | O | H | Me | (pyrimidin-2-ylsulphonyl) | 0.74 |
| | | | | | | | | methyl | |
| I-1-237 | | C—H | C—H | H | O | H | Me | (2,2-dichloro- | 1.84 |
| | | | | | | | | cyclopropyl)methyl | |
| I-1-238 | | C—H | C—H | H | O | H | Me | 3,3-dichloroprop-2-en-1-yl | 1.83 |
| I-1-239 | | C—H | C—H | H | O | H | Me | 3-methylbut-2-en-1-yl | 1.75 |
| I-1-240 | | N | C—H | H | O | H | Me | 1H-pyrazol-1-ylmethyl | 0.91 |
| I-1-241 | | C—H | C—H | H | O | H | Me | (methylsulphanyl)methyl | 1.17 |
| I-1-242 | | C—H | C—H | H | O | H | Me | (ethylsulphanyl)methyl | 1.49 |
| I-1-243 | | C—H | C—H | H | O | H | Me | (methylsulphinyl)methyl | 0.19 |
| I-1-244 | | C—H | C—H | H | O | H | Me | (methylsulphonyl)methyl | 0.59 |
| I-1-245 | | C—H | C—H | H | O | H | Me | (ethylsulphinyl)methyl | 0.45 |
| I-1-246 | | C—H | C—H | H | O | H | Me | (ethylsulphonyl)methyl | 0.83 |
| I-1-247 | | C—H | C—H | H | O | H | Me | 3-(2-chlorophenyl)-2- | 1.92 |
| | | | | | | | | oxopropyl | |
| I-1-248 | | N | C—H | H | O | H | Me | 4,6-dimethylpyrimidin-2-yl | 1.53 |
| I-1-249 | | N | C—H | H | O | H | Me | 2-pyridyl | 1.83 |
| I-1-250 | | N | C—H | H | O | H | Me | 2,2,2-trifluoroethyl | 1.2 |
| I-1-251 | | N | C—H | H | O | H | Me | (4,6-dimethylpyrimidin-2- | 1.14 |
| | | | | | | | | yl)methyl | |
| I-1-252 | | N | C—H | H | O | H | Me | (4,6-dimethoxypyrimidin- | 1.5 |
| | | | | | | | | 2-yl)methyl | |
| I-1-253 | | N | C—H | H | O | H | Me | (4-methylpyrimidin-2- | 0.88 |
| | | | | | | | | yl)methyl | |
| I-1-254 | | N | C—H | H | O | H | Me | (3-chloropyridin-2- | 1.33 |
| | | | | | | | | yl)methyl | |
| I-1-255 | | N | C—H | H | O | H | Me | [3-(trifluoromethyl)-1H- | 1.73 |
| | | | | | | | | pyrazol-1-yl]methyl | |
| I-1-256 | | N | C—H | H | O | H | Me | (4-methyl-1H-pyrazol-1- | 1.1 |
| | | | | | | | | yl)methyl | |
| I-1-257 | | C—H | C—H | H | O | H | Me | methylsulphamoyl | 0.55 |
| I-1-258 | | C—H | C—H | H | O | H | Me | propylsulphamoyl | 1.07 |
| I-1-259 | | C—H | C—H | H | O | H | Me | (2,2,2-trifluoroethoxy)- | 1.79 |
| | | | | | | | | carbonyl | |
| I-1-260 | | C—H | C—H | H | O | H | Me | 2-(1-chlorocyclopropyl)-2- | 1.38 |
| | | | | | | | | oxoethyl | |
| I-1-261 | | C—H | C—H | H | O | H | Me | methoxyacetyl | 1.17 |
| I-1-262 | | C—H | C—H | cyano | O | H | Me | pyrimidin-2-ylmethyl | 0.84 |
| | | | | methyl | | | | | |
| I-1-263 | | C—H | C—H | H | O | H | Me | methyl(phenylsulphonyl) | 2.13 |
| | | | | | | | | carbamoyl | |
| I-1-264 | | C—H | C—H | prop- | O | H | Me | pyrimidin-2-ylmethyl | 0.98 |
| | | | | 2-yn- | | | | | |
| | | | | 1-yl | | | | | |
| I-1-265 | | C—H | C—H | 2,2- | O | H | Me | pyrimidin-2-ylmethyl | 1.19 |
| | | | | difluoro- | | | | | |
| | | | | ethyl | | | | | |
| I-1-266 | | C—H | C—H | H | O | H | Me | 3-(methylsulphanyl)- | 1.87 |
| | | | | | | | | propanoyl | |
| I-1-267 | | C—H | C—H | H | O | F | H | pyrimidin-2-ylmethyl | 0.42 |
| I-1-268 | | C—H | C—H | H | O | H | Me | 3-(methylsulphonyl) | 1.04 |
| | | | | | | | | propanoyl | |
| I-1-269 | | C—H | C—H | H | O | H | CN | pyrimidin-2-ylmethyl | 1.0 |
| I-1-270 | | C—H | C—H | H | O | CN | SMe | CH₃ | 0.96 |
| I-1-271 | | C—H | C—H | H | O | H | H | 4-methoxyphenyl | 1.6 |
| I-1-272 | | C—H | C—H | H | O | COOEt | H | benzyl | 0.79 |
| I-1-273 | | C—H | C—H | H | O | H | H | 1,3-thiazol-2-yl | 1.16 |
| I-1-274 | | C—H | C—H | H | O | H | Cl | phenyl | 2.1 |
| I-1-275 | | C—H | C—H | H | O | Cl | OCHF₂ | CH₃ | 1.19 |
| I-1-276 | | C—H | C—Cl | H | O | H | Me | pyrimidin-2-ylmethyl | 1.4 |
| I-1-277 | | C—H | C—H | H | O | H | Me | SO₂CH₃ | 1.03 |
| I-1-278 | | C—H | C—H | H | O | H | Me | ethylsulphonyl | 1.29 |
| I-1-279 | | C—H | C—H | H | O | H | Me | pyridin-2-ylsulphonyl | 1.51 |
| I-1-280 | | C—H | C—H | H | O | H | Me | pyridin-3-ylsulphonyl | 1.45 |
| I-1-281 | | C—H | C—H | H | O | H | Me | (5-tert-butyl-1,3,4- | 1.44 |
| | | | | | | | | oxadiazol-2-yl)methyl | |
| I-1-282 | | C—H | C—H | H | O | H | Me | (5-methyl-1,2,4-oxadiazol- | 0.9 |
| | | | | | | | | 3-yl)methyl | |
| I-1-283 | | C—H | C—H | H | O | H | Me | (5-methyl-1,2-oxazol-3- | 1.19 |
| | | | | | | | | yl)methyl | |
| I-1-284 | | C—H | C—H | H | O | H | Me | (3-methyl-1,2,4-oxadiazol- | 0.92 |
| | | | | | | | | 5-yl)methyl | |
| I-1-285 | | C—H | C—H | H | O | H | Me | (5-tert-butyl-1,2,4- | 1.71 |
| | | | | | | | | oxadiazol-3-yl)methyl | |
| I-1-286 | | C—H | C—H | H | O | H | Me | [5-(dimethylcarbamoyl)- | 1.03 |
| | | | | | | | | 1,2,4-oxadiazol-3- | |
| | | | | | | | | yl]methyl | |
| I-1-287 | | C—H | C—H | H | O | H | Me | isopropylcarbamoyl | 1.46 |
| I-1-288 | | C—H | C—H | H | O | H | Me | ethylcarbamoyl | 1.24 |
| I-1-289 | | C—H | C—H | H | O | H | Me | (ethylsulphonyl)(methyl)- | 1.5 |
| | | | | | | | | carbamoyl | |
| I-1-290 | | C—H | C—H | H | O | H | Me | diethylcarbamoyl | 2.31 |
| I-1-291 | | C—H | C—H | H | O | H | Me | dimethylcarbamoyl | 0.86 |
| I-1-292 | | C—H | C—H | H | O | H | Me | CONHCH₃ | 0.97 |
| I-1-293 | | C—H | C—H | H | O | H | Me | (1-methyl-1H-imidazol-2- | −0.21 |
| | | | | | | | | yl)methyl | |
| I-1-294 | | C—H | C—H | H | O | H | Me | 2,2-difluoro-2-(5- | 1.77 |
| | | | | | | | | fluorpyridin-2-yl)ethyl | |
| I-1-295 | | C—H | C—H | H | O | H | Me | 1,3-thiazol-4-ylmethyl | 0.95 |
| I-1-296 | | C—H | C—H | H | O | H | Me | (5-methyl-1,3,4-thiadiazol- | 0.83 |
| | | | | | | | | 2-yl)methyl | |
| I-1-297 | | C—H | C—H | H | O | H | Me | (5-chloro-2-thienyl)methyl | 2.07 |
| I-1-298 | | C—H | C—H | H | O | H | Me | [3-(dimethylcarbamoyl)- | 1.09 |
| | | | | | | | | 1,2-oxazol-5-yl]methyl | |
| I-1-299 | | C—H | C—H | H | O | H | Me | (5-phenyl-1,3,4-oxadiazol- | 1.57 |
| | | | | | | | | 2-yl)methyl | |
| I-1-300 | | C—H | C—H | H | O | H | Me | (3-phenyl-1,2,4-oxadiazol- | 2.01 |
| | | | | | | | | 5-yl)methyl | |
| I-1-301 | | C—H | C—H | H | O | H | Me | (5,6-dichloropyridin-2- | 2.06 |
| | | | | | | | | yl)methyl | |
| I-1-302 | | C—H | C—H | H | O | H | Me | [2-(trifluoromethyl)-1,3- | 1.93 |
| | | | | | | | | thiazol-4-yl]methyl | |
| I-1-303 | | C—H | C—H | H | O | H | Me | (5-methyl-2-phenyl-2H- | 2.33 |
| | | | | | | | | 1,2,3-triazol-4-yl)methyl | |
| I-1-304 | | C—H | C—H | H | O | H | Me | (1-phenyl-1H-1,2,4-triazol- | 1.32 |
| | | | | | | | | 5-yl)methyl | |
| I-1-305 | | C—H | C—H | (3-phenyl- | O | H | Me | (3-phenyl-1,2,4-oxadiazol- | 3.26 |
| | | | | 1,2,4- | | | | 5-yl)methyl | |
| | | | | oxadiazol- | | | | | |
| | | | | 5-yl) | | | | | |
| | | | | methyl | | | | | |
| I-1-306 | | C—H | C—H | H | O | H | Me | (Z)-2-(5-chloropyridin-2- | 2.31 |
| | | | | | | | | yl)-2-fluorovinyl | |
| I-1-307 | | C—H | C—H | H | O | H | Me | (1,1,1-trifluoropropan-2- | 1.89 |
| | | | | | | | | yl)carbamoyl | |
| I-1-308 | | C—H | C—H | H | O | H | Me | [2-(methylsulphinyl)- | 0.84 |
| | | | | | | | | ethyl]carbamoyl | |
| I-1-309 | | C—H | C—H | H | O | H | Me | [2-(methylsulphonyl)- | 0.97 |
| | | | | | | | | ethyl]carbamoyl | |
| I-1-310 | | C—H | C—H | H | O | H | Me | (1,4-dioxan-2- | 1.2 |
| | | | | | | | | ylmethyl)carbamoyl | |
| I-1-311 | | C—H | C—H | H | O | H | Me | (tetrahydrofuran-2- | 1.45 |
| | | | | | | | | ylmethyl)carbamoyl | |
| I-1-312 | | C—H | C—H | H | O | H | Me | cyclopropylcarbamoyl | 1.27 |
| I-1-313 | | C—H | C—H | H | O | H | Me | (tetrahydro-2H-pyran-2- | 1.78 |
| | | | | | | | | ylmethyl)carbamoyl | |
| I-1-314 | | C—H | C—H | H | O | H | Me | propylcarbamoyl | 1.56 |
| I-1-315 | | C—H | C—H | H | O | H | Me | butylcarbamoyl | 1.92 |
| I-1-316 | | C—H | C—H | H | O | H | Me | (cyclopropylmethyl)- | 1.65 |
| | | | | | | | | carbamoyl | |
| I-1-317 | | C—H | C—H | H | O | H | Me | sec-butylcarbamoyl | 1.83 |
| I-1-318 | | C—H | C—H | H | O | H | Me | n-pentan-2-ylcarbamoyl | 2.18 |
| I-1-319 | | C—H | C—H | H | O | H | Me | tert-butylcarbamoyl | 1.99 |
| I-1-320 | | C—H | C—H | H | O | H | Me | isobutylcarbamoyl | 1.89 |
| I-1-321 | | C—H | C—H | H | O | H | Me | (2,2-dimethylpropyl)- | 2.19 |
| | | | | | | | | carbamoyl | |
| I-1-322 | | C—H | C—H | H | O | H | Me | benzylcarbamoyl | 1.97 |
| I-1-323 | | C—H | C—H | H | O | H | Me | (2-chlorobenzyl)carbamoyl | 2.29 |
| I-1-324 | | C—H | C—H | H | O | H | Me | (3-chlorobenzyl)carbamoyl | 2.39 |
| I-1-325 | | C—H | C—H | H | O | H | Me | (4-chlorobenzyl)carbamoyl | 2.34 |
| I-1-326 | | C—H | C—H | H | O | H | Me | (2-fluorobenzyl)carbamoyl | 2.04 |
| I-1-327 | | C—H | C—H | H | O | H | Me | (3-fluorobenzyl)carbamoyl | 2.1 |
| I-1-328 | | C—H | C—H | H | O | H | Me | (4-fluorobenzyl)carbamoyl | 2.12 |
| I-1-329 | | C—H | C—H | H | O | H | Me | (pyridin-2-ylmethyl)- | 0.88 |
| | | | | | | | | carbamoyl | |
| I-1-330 | | C—H | C—H | H | O | H | Me | (pyridin-3-ylmethyl)- | 0.64 |
| | | | | | | | | carbamoyl | |
| I-1-331 | | C—H | C—H | H | O | H | Me | (1-phenylethyl)carbamoyl | 2.22 |
| I-1-332 | | C—H | C—H | H | O | H | Me | [1-(4-fluorophenyl)- | 2.28 |
| | | | | | | | | ethyl]carbamoyl | |
| I-1-333 | | C—H | C—H | H | O | H | Me | [1-(pyrimidin-2- | 1.29 |
| | | | | | | | | yl)ethyl]carbamoyl | |
| I-1-334 | | C—H | C—H | H | O | H | Me | (pyrimidin-2- | 0.95 |
| | | | | | | | | ylmethyl)carbamoyl | |
| I-1-335 | | C—H | C—H | H | O | H | Me | [2-(pyrimidin-2-yl)propan- | 1.67 |
| | | | | | | | | 2-yl]carbamoyl | |
| I-1-336 | | C—H | C—H | H | O | H | Me | (pyrimidin-4- | 1.05 |
| | | | | | | | | ylmethyl)carbamoyl | |
| I-1-337 | | C—H | C—H | H | O | H | Me | 2-fluoro-4-methyl-5- | 2.89 |
| | | | | | | | | [(2,2,2-trifluoroethyl)- | |
| | | | | | | | | sulphanyl]benzyl | |
| I-1-338 | | C—H | C—H | H | O | H | Me | [(4,6-dimethylpyrimidin-2- | 1.43 |
| | | | | | | | | yl)methyl]carbamoyl | |
| I-1-339 | | C—H | C—H | H | O | H | Me | 2,1,3-benzothiadiazol-5- | 1.61 |
| | | | | | | | | ylmethyl | |
| I-1-340 | | C—H | C—H | H | O | H | Me | (5-chloro-1,3-benzoxazol- | 2 |
| | | | | | | | | 2-yl)methyl | |
| I-1-341 | | C—H | C—H | H | O | H | Me | quinolin-2-ylmethyl | 1.66 |
| I-1-342 | | C—H | C—H | H | O | H | Me | (6-chloro-1,3-benzoxazol- | 1.82 |
| | | | | | | | | 2-yl)methyl | |
| I-1-343 | | C—H | C—H | 2,1,3- | O | H | Me | 2,1,3-benzothiadiazol-5- | 2.57 |
| | | | | benzothia- | | | | ylmethyl | |
| | | | | diazol-5- | | | | | |
| | | | | ylmethyl | | | | | |
| I-1-344 | | C—H | C—H | H | S | H | Me | pyrimidin-2-ylmethyl | 1.24 |
| I-1-345 | | C—H | C—H | H | O | H | Me | pyrimidin-2-ylcarbonyl | 1.11 |
| I-1-346 | | C—H | C—H | Me | O | H | Me | pyrimidin-2-ylmethyl | 0.63 |
| I-1-347 | | C—H | C—H | H | O | H | Me | t-butyl | 1.61 |
| I-1-348 | | C—H | C—F | H | O | H | Me | t-butyl | 2.13 |
| I-1-349 | | C—H | C—H | H | O | H | Me | phenyl | 1.7 |
| I-1-350 | | C—H | C—F | H | O | H | Me | phenyl | 2.18 |
| I-1-351 | | C—H | C—H | H | O | H | Me | (6-chloroimidazo[1,2- | 1.42 |
| | | | | | | | | b]pyridazin-2-yl)methyl | |
| I-1-352 | | C—H | C—H | H | O | H | Me | (2-ethyl-5-oxo-5H- | 1.23 |
| | | | | | | | | [1,3,4]thiadiazolo[3,2- | |
| | | | | | | | | a]pyrimidin-7-yl)methyl | |
| I-1-353 | | C—H | C—H | H | O | H | Me | (5-methyl-1,3-benzoxazol- | 1.9 |
| | | | | | | | | 2-yl)methyl | |

Compounds of the formula (1-2)

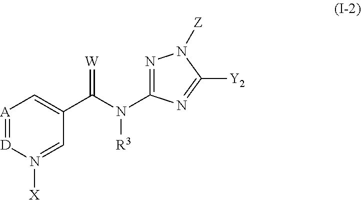

in which X is a free electron pair, and A, D, W, R³, Y₂ and Z are each as defined in Table 2.

TABLE 2

| Compd. | | | | | | | log P |
|---|---|---|---|---|---|---|---|
| No. | D | A | R³ | W | Y₂ | Z | (acid)¹ |
| I-2-1 | C—H | C—H | H | O | methyl | H | <−0.23 |
| I-2-2 | C—H | C—H | H | O | methyl | pyrimidin-2-ylmethyl | −0.05 |
| I-2-3 | C—H | C—H | H | O | H | 2,6-dichloro-4-[(trifluoromethyl)- | 2.02 |
| | | | | | | sulphonyl]phenyl | |
| I-2-4 | C—H | C—H | H | O | H | 2-methoxy-5-nitrobenzyl | 1.13 |
| I-2-5 | C—H | C—H | H | O | H | phenyl | 0.98 |
| I-2-6 | C—H | C—H | H | O | ethyl | phenyl | 1.24 |
| I-2-7 | C—H | C—H | H | O | H | 2-chlorobenzyl | 1.22 |
| I-2-8 | C—H | C—H | H | O | H | 2,6-dichlorobenzyl | 1.39 |
| I-2-9 | C—H | C—H | H | O | H | dimethylcarbamoyl | 0.47 |
| I-2-10 | C—H | C—H | H | O | H | 4,6-diethoxy-1,3,5-triazin-2-yl | 1.41 |
| I-2-11 | C—H | C—H | H | O | H | 4-methoxypyrimidin-2-yl | 0.70 |
| I-2-12 | C—H | C—H | H | O | SCH₃ | ethyl | 0.71 |
| I-2-13 | C—H | C—H | H | O | H | 4-chlorobenzyl | 1.36 |
| I-2-14 | C—H | C—H | H | O | H | methyl | <−0.23 |
| I-2-15 | C—H | C—H | H | O | H | 3-bromopyridin-2-yl | 0.67 |
| I-2-16 | C—H | C—H | H | O | OCH₃ | methyl | 0.00 |
| I-2-17 | C—H | C—H | H | O | ethyl | methyl | 0.01 |
| I-2-18 | C—H | C—H | H | O | H | CH₂OMe | −0.23 |
| I-2-19 | C—H | C—H | H | O | H | 3-chlorobenzyl | 1.33 |
| I-2-20 | C—H | C—H | H | O | methyl | i-propyl | 0.44 |
| I-2-21 | C—H | C—H | H | O | H | n-propyl | 0.29 |
| I-2-22 | C—H | C—H | H | O | H | 4,6-dimethylpyrimidin-2-yl | 0.86 |
| I-2-23 | C—H | C—H | H | O | H | 2-methylbenzyl | 1.23 |
| I-2-24 | C—H | C—H | H | O | H | 4,6-dimethoxypyrimidin-2-yl | 1.11 |
| I-2-25 | C—H | C—H | H | O | H | 4,5,6-trimethylpyrimidin-2-yl | 1.07 |
| I-2-26 | C—H | C—H | H | O | SCH₃ | methyl | 0.32 |
| I-2-27 | C—H | C—H | H | O | Cl | phenyl | 1.58 |
| I-2-28 | C—H | C—H | H | O | OMe | phenyl | 1.23 |

Compounds of the formula (1-3)

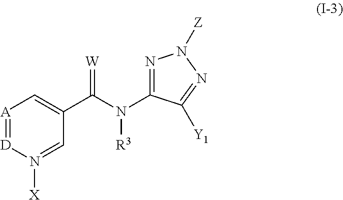

in which X is a free electron pair, and A, D, W, R³, Y₁ and Z are each as defined in Table 3.

TABLE 3

| Compd. | | | | | | | log P |
|---|---|---|---|---|---|---|---|
| No. | D | A | R³ | W | Y₁ | Z | (acid)¹ |
| I-3-1 | C—H | C—H | H | O | methyl | pyridin-2-yl | 1.08 |
| I-3-2 | C—H | C—H | H | O | H | CH₃ | 0.38 |
| I-3-3 | C—H | C—H | H | O | Br | phenyl | 2.09 |
| I-3-4 | C—H | C—H | H | O | H | 4-chlorophenyl | 2.43 |
| I-3-5 | C—H | C—H | H | O | H | 3-chloro-5-(trifluoromethyl)pyridin-2-yl | 2.12 |
| I-3-6 | C—H | C—H | H | O | CF₃ | 3-chloropyridin-2-yl | 1.89 |
| I-3-7 | C—H | C—H | H | O | H | 3-bromopyridin-2-yl | 1.33 |
| I-3-8 | C—H | C—H | H | O | CF₃ | 2,4-dichlorophenyl | 2.97 |
| I-3-9 | C—H | C—H | H | O | Me | 3-chloropyridin-2-yl | 1.24 |
| I-3-10 | C—H | C—H | H | O | H | i-propyl | 1.09 |
| I-3-11 | C—H | C—H | H | O | Me | phenyl | 1.82 |
| I-3-12 | C—H | C—H | H | O | CF₃ | phenyl | 2.44 |
| I-3-13 | C—H | C—H | H | O | Cl | 2-chlorophenyl | 2.04 |
| I-3-14 | C—H | C—H | H | O | H | 5-(trifluoromethyl)pyridin-2-yl | 1.91 |
| I-3-15 | C—H | C—H | H | O | H | 3,5-dichloropyridin-2-yl | 1.83 |
| I-3-16 | C—H | C—H | H | O | H | 3-chloropyridin-2-yl | 1.32 |
| I-3-17 | C—H | C—H | H | O | CH₃ | 3,5-dichloropyridin-2-yl | 1.74 |
| I-3-18 | C—H | C—H | H | O | CH₃ | 3-(trifluoromethyl)phenyl | 2.58 |
| I-3-19 | C—H | C—H | H | O | CH₃ | 2,4-dichlorophenyl | 2.24 |
| I-3-20 | C—H | C—H | H | O | CF₃ | 4-chlorophenyl | 2.93 |
| I-3-21 | C—H | C—H | H | O | Cl | 3-chloropyridin-2-yl | 1.51 |
| I-3-22 | C—H | C—H | H | O | Me | 2,5-dichlorophenyl | 2.26 |
| I-3-23 | C—H | C—H | H | O | Me | 2-chlorophenyl | 1.71 |
| I-3-24 | C—H | C—H | H | O | H | 2,6-difluorophenyl | 1.67 |

Compounds of the formula (1-4)

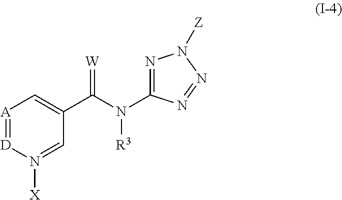

in which X is a free electron pair, and A, D, W, R³ and Z are each as defined in Table 4.

TABLE 4

| Compd. | | | | | | log P |
|---|---|---|---|---|---|---|
| No. | D | A | R³ | W | Z | (acid)¹ |
| I-4-1 | C—H | C—H | H | O | ethyl | — |
| I-4-2 | C—H | C—H | H | O | CH₃ | <−0.23 |
| I-4-3 | C—H | C—H | H | O | (6-chloropyridin-3-yl)methyl | 1.05 |
| I-4-4 | C—H | C—H | H | O | 4,6-dimethoxypyrimidin-2-yl | 1.35 |
| I-4-5 | C—H | C—H | H | O | 2-ethoxy-2-oxoethyl | 0.71 |
| I-4-6 | C—H | C—H | H | O | 2-oxo-2-[(1-phenylethyl)amino]ethyl | 1.34 |
| I-4-7 | C—H | C—H | H | O | 2-[(1-naphthylmethyl)amino]-2-oxoethyl | 1.71 |
| I-4-8 | C—H | C—H | H | O | allyl | 0.60 |
| I-4-9 | C—H | C—H | H | O | n-propyl | 0.76 |
| I-4-10 | C—H | C—H | H | O | benzyl | 1.38 |
| I-4-11 | C—H | C—H | H | O | 2-(butylamino)-2-oxoethyl | 0.97 |

¹The logP values were measured according to EEC Directive 79/831 Annex V.A8 by HPLC (high-performance liquid chromatography) on a reversed-phase column (C 18). Temperature: 55° C.

The LC-MS determination in the acidic range is effected at pH 2.7 using 0.1% aqueous formic acid and acetonitrile (contains 0.1% formic acid) as eluents, linear gradient from 10% acetonitrile to 95% acetonitrile. Alternatively, the determination is effected in the acidic range at pH 2.3 using 0.1% aqueous phosphoric acid and acetonitrile as eluents; linear gradient from 10% acetonitrile to 95% acetonitrile.

Calibration is effected with unbranched alkan-2-ones (having 3 to 16 carbon atoms) with known log P values (log P values determined on the basis of the retention times by linear interpolation between two successive alkanones).

### NMR Data for Selected Examples²⁾

Compound No. I-1-25 (Synthesis Example 14): 1H NMR (DMSO-D₆) 2.53 (s, 3H), 3.43 (s, 3H), 6.11 (br. s, 1H),
7.30-7.33 (m, 2H), 7.79-7.82 (m, 1H), 8.56-8.67 (m, 2H), 8.76-8.77 (m, 2H).
Compound No. I-1-131, solvent: [CDCl3], spectrometer: 600.13 MHz
9.1353 (2.45); 9.1324 (2.42); 8.8222 (1.68); 8.82 (1.73); 8.8143 (1.76); 8.8122 (1.64); 8.412 (1.52); 8.2381 (1.24);
8.2351 (1.75); 8.2315 (1.22); 8.2249 (1.35); 8.2218 (1.82); 8.2183 (1.21); 7.4794 (1.48); 7.4713 (1.53); 7.4662
(1.52); 7.4582 (1.42); 7.2618 (20.87); 6.8688 (3.56); 3.018 (3.82); 3.0057 (6.42); 3 (0.56); 2.9932 (4.04); 2.6262
(16); 2.625 (15.51); 2.3081 (0.54); 2.0455 (0.33); 1.8123 (0.49); 1.7999 (2.18); 1.7876 (4.17); 1.7816 (0.43);
1.7752 (4.16); 1.7628 (2.19); 1.7505 (0.5); 1.6059 (2.23); 1.3326 (0.75); 1.2844 (1.01); 1.2714 (0.33); 1.2543
(1.84); 1.0586 (0.35); 1.0443 (6.17); 1.032 (12.22); 1.0196 (5.8); 0.8801 (0.34); 0.0697 (2.45); 0.0052 (0.65); −0.0001
(14.55); −0.0056 (0.61)
Compound No. I-1-130, solvent: [CDCl3], spectrometer: 600.13 MHz
9.1456 (1.87); 9.1426 (1.83); 8.8217 (1.31); 8.8192 (1.34); 8.8138 (1.36); 8.8112 (1.27); 8.5293 (1.07); 8.2486
(0.83); 8.2455 (1.16); 8.2421 (0.8); 8.2354 (0.88); 8.2323 (1.19); 8.2289 (0.78); 7.4796 (1); 7.4715 (1.04); 7.4664
(1.02); 7.4583 (0.96); 7.2645 (5.13); 6.8776 (2.33); 3.7868 (0.42); 3.7753 (1.08); 3.7638 (1.46); 3.7523 (1.1);
3.7408 (0.44); 2.6204 (10.39); 2.6192 (9.95); 2.0456 (0.35); 1.7042 (0.83); 1.2846 (0.5); 1.27 (15.75); 1.2585 (16);
1.2475 (0.39); 0.0708 (0.5); −0.0001 (3.64)
Compound No. I-1-129, solvent: [CDCl3], spectrometer: 600.13 MHz
9.1449 (2.75); 8.8175 (1.92); 8.8108 (1.93); 8.5713 (1.88); 8.2413 (1.82); 8.2281 (1.87); 7.4778 (1.49); 7.4697
(1.62); 7.4648 (1.59); 7.4567 (1.4); 7.2653 (4.96); 6.8694 (3.8); 3.0639 (1.63); 3.0516 (4.88); 3.0393 (4.95); 3.027
(1.68); 2.6252 (16); 1.7875 (0.36); 1.2561 (5.49); 1.2438 (9.98); 1.2315 (4.96); 0.0711 (0.71); −0.0001 (3.52)
Compound No. I-1-121 (Synthesis Example 8), solvent: [DMF], spectrometer: 601.6 MHz
11.4702 (6.65); 9.3267 (10.56); 9.3255 (10.81); 9.3229 (10.95); 9.3218 (10.11); 8.8237 (8.74); 8.821 (9.21);
8.8157 (9.23); 8.813 (9.04); 8.5224 (5.53); 8.5195 (6.91); 8.5189 (6.42); 8.5158 (5.41); 8.5092 (5.83); 8.5062 (7);
8.5054 (6.87); 8.5025 (5.38); 8.3676 (1.02); 8.0268 (8.21); 7.6214 (6.33); 7.6201 (5.97); 7.6135 (6.21); 7.6122
(6.04); 7.6082 (6.32); 7.6069 (6.03); 7.6003 (6.05); 7.599 (5.65); 6.9658 (16); 6.9642 (15.53); 3.479 (10.85);
3.1044 (1.8); 3.0966 (4.03); 3.0912 (4.3); 3.0891 (2.81); 3.0834 (7.77); 3.0786 (2.36); 3.0757 (4.44); 3.0703
(4.28); 3.0626 (2.03); 2.9524 (7.84); 2.922 (4.07); 2.9189 (8.25); 2.9158 (11.65); 2.9126 (8.18); 2.9095 (3.83);
2.7852 (7.73); 2.7521 (4.55); 2.7489 (9.01); 2.7457 (13.1); 2.7425 (9.08); 2.7392 (4.48); 2.6904 (0.33); 2.633
(0.36); 2.6145 (0.34); 2.5838 (58.74); 2.5823 (59.79); 2.4736 (0.34); 2.4721 (0.33); 2.3282 (0.34); 2.0138 (0.88);
1.3793 (1.67); 1.2967 (2.36); 1.2765 (1.37); 1.1997 (0.51); 1.1701 (2.67); 1.1661 (3.39); 1.1627 (6.72); 1.1575
(14.41); 1.1528 (6.67); 1.1494 (6.39); 1.1444 (12.15); 1.1392 (5.49); 1.1295 (2.51); 1.1146 (4.88); 1.1097 (12.56);
1.1074 (6.13); 1.1064 (6.7); 1.1045 (8.39); 1.102 (14.4); 1.097 (8.48); 1.0928 (2.95); 1.0899 (2.46); 0.0052 (0.47);
−0.0002 (14.23); −0.0058 (0.44)
Compound No. I-1-126, solvent: [DMSO], spectrometer: 399.95 MHz
11.5431 (2.28); 10.8427 (0.56); 9.1475 (2.09); 9.1423 (2.07); 9.0744 (0.62); 9.0697 (0.61); 8.8372 (0.41); 8.8334
(0.43); 8.825 (0.43); 8.8205 (0.42); 8.7591 (1.48); 8.7551 (1.54); 8.7471 (1.52); 8.7432 (1.49); 8.369 (0.87);
8.3642 (1.24); 8.3591 (0.84); 8.349 (0.9); 8.3439 (1.3); 8.3393 (0.81); 8.3155 (0.55); 8.2626 (0.33); 8.2422 (0.37);
7.6516 (0.33); 7.6405 (0.32); 7.6329 (0.34); 7.6203 (0.32); 7.5566 (1.12); 7.5452 (1.14); 7.5364 (1.13); 7.5246
(1.06); 6.8599 (3.08); 6.6595 (1.36); 5.7555 (0.99); 3.9801 (4.65); 3.9407 (16); 3.3209 (39.7); 2.6751 (0.92);
2.6706 (1.27); 2.6659 (0.9); 2.5246 (15.38); 2.5101 (76.95); 2.506 (146.69); 2.5015 (188.94); 2.4971 (135.14);
2.493 (64.86); 2.3329 (0.85); 2.3282 (1.17); 2.3238 (0.88); 2.226 (4.09); 1.9884 (0.75); 1.2585 (0.45); 1.2341
(0.59); 1.1748 (0.41); 0.146 (0.44); 0.0079 (4.34); −0.0002 (99.88); −0.0085 (3.51); −0.1498 (0.47)
Compound No. I-1-127, solvent: [DMSO], spectrometer: 399.95 MHz
12.5148 (2.08); 8.9062 (3.17); 8.9023 (3.1); 8.7071 (2.27); 8.7031 (2.39); 8.695 (2.4); 8.691 (2.29); 8.3159 (1.1);
8.1252 (1.34); 8.1209 (1.85); 8.1154 (1.32); 8.1053 (1.51); 8.0998 (1.95); 8.0954 (1.37); 7.7921 (2.32); 7.7877
(3.77); 7.7834 (2.51); 7.7342 (1.61); 7.7317 (2.07); 7.7278 (1.51); 7.7149 (1.92); 7.7122 (2.35); 7.7085 (1.68);
7.6381 (1.45); 7.6356 (1.66); 7.6328 (1.51); 7.6302 (1.42); 7.6179 (1.97); 7.6154 (2.07); 7.6126 (2.06); 7.6102
(1.7); 7.5121 (1.77); 7.5103 (1.78); 7.4999 (4.1); 7.4923 (1.84); 7.4904 (1.8); 7.4801 (5.24); 7.4603 (1.66); 6.1543
(3.27); 6.151 (3.23); 4.0554 (0.67); 4.0376 (1.96); 4.0199 (1.96); 4.0021 (0.62); 3.3215 (206.98); 2.675 (2.46);
2.6705 (3.31); 2.666 (2.45); 2.5408 (2.59); 2.5237 (10.78); 2.5103 (199.81); 2.5059 (388.58); 2.5014 (498.01);
2.4969 (358.02); 2.4925 (170.77); 2.3328 (2.36); 2.3282 (3.13); 2.3237 (2.23); 2.2278 (0.78); 2.2141 (0.38);
2.1856 (16); 1.9886 (8.67); 1.298 (3.13); 1.2811 (0.53); 1.2586 (4.67); 1.2351 (5.43); 1.1926 (2.59); 1.1748 (4.92);
1.157 (2.46); 0.8842 (0.36); 0.8674 (0.66); 0.8537 (0.99); 0.8366 (0.55); 0.146 (2.54); 0.0215 (1.17); 0.008
(24.33); −0.0001 (613.66); −0.0084 (23.69); −0.1496 (2.55)
Compound No. I-1-8: mass (m/z): 253.1 (M + H)⁺; 1H NMR (CD₃CN) 2.44 (s, 3H), 6.74 (s, 1H), 7.08-7.38 (m,
1H), 7.44-7.47 (m, 1H), 8.20-8.23 (m, 1H), 8.72-8.73 (m, 1H), 9.06 (s, 1H), 9.13 (br. s, 1H).
Compound No. I-1-122 (Synthesis Example 6): HPLC-MS: logP = 2.27; mass (m/z): 341.0 (M + H)⁺; ¹H NMR
(DMSO-D₆) 2.68 (s, 3H), 7.01 (s, 1H), 7.45-7.59 (m, 4H), 7.64-7.66 (m, 1H), 8.26-8.29 (m, 1H),
8.70-8.72 (m, 1H), 9.05 (s, 1H), 11.41 (s, 1H), 13C NMR (DMF-D7) 15.2, 107.0, 124.9, 128.5, 130.9, 131.0, 132.0, 133.2,
136.9, 137.1, 145.9, 150.8, 152.3, 154.2, 166.0, 168.4.
Compound No. I-1-28, solvent: [DMSO], spectrometer: 399.95 MHz
11.3961 (2.16); 11.2451 (2.58); 9.1109 (4.38); 9.1065 (4.28); 9.0984 (2.24); 9.097 (2.29); 9.0931 (2.2); 9.0748
(2.74); 9.0724 (2.68); 9.0694 (2.62); 8.8513 (1.88); 8.8473 (1.95); 8.8393 (2.03); 8.8352 (2.02); 8.8305 (1.95);
8.8263 (1.97); 8.8183 (1.98); 8.8141 (1.92); 8.7995 (1.11); 8.7952 (1.15); 8.788 (2.35); 8.7838 (2.31); 8.7761
(1.69); 8.772 (1.69); 8.7538 (1.65); 8.7497 (1.7); 8.7418 (1.7); 8.7377 (1.62); 8.3568 (1.14); 8.3517 (1.57); 8.3469
(1.08); 8.3363 (1.7); 8.3314 (2.71); 8.3269 (1.58); 8.3154 (1.07); 8.3098 (1.6); 8.3075 (1.66); 8.302 (2.3); 8.2973
(2.26); 8.292 (1.18); 8.2875 (1.46); 8.282 (2.62); 8.2776 (2.65); 8.2723 (1.07); 8.2645 (0.74); 8.2593 (0.95);
8.2546 (0.59); 7.6707 (1.3); 7.669 (1.3); 7.6586 (1.31); 7.657 (1.31); 7.6508 (1.28); 7.6491 (1.27); 7.6377 (2.39);
7.6255 (1.36); 7.6238 (1.35); 7.6178 (1.3); 7.616 (1.29); 7.6056 (1.41); 7.6029 (1.78); 7.6004 (1.24); 7.5899
(1.17); 7.5881 (1.16); 7.5823 (1.15); 7.5804 (1.15); 7.5701 (1.17); 7.5682 (1.21); 7.5652 (1.02); 7.5632 (0.96);
7.5528 (0.95); 7.5509 (1.02); 7.5453 (1.92); 7.5332 (1.81); 7.5272 (1.15); 7.5255 (1.09); 7.5151 (1.04); 7.5133
(1.01); 7.0048 (2.58); 7.0028 (2.47); 6.8275 (5.57); 4.0385 (0.63); 4.0207 (0.63); 3.3332 (5.42); 3.0885 (0.32);
2.6717 (9.36); 2.6699 (9.07); 2.6492 (0.41); 2.6275 (0.34); 2.5257 (1.12); 2.5125 (18.31); 2.5081 (35.47); 2.5035
(45.71); 2.499 (32.73); 2.4945 (15.61); 2.3303 (0.46); 2.2555 (16); 2.0746 (1.2); 2.0394 (0.92); 1.9898 (2.89);
1.2008 (1.03); 1.1931 (0.85); 1.1826 (2.11); 1.1753 (1.64); 1.1643 (1.03); 1.1576 (0.83); 0.008 (0.38); −0.0002
(10.56); −0.0085 (0.39)
Compound No. I-1-118 (Synthesis Example 1), solvent: [DMSO], spectrometer: 399.95 MHz
11.186 (6.01); 8.9279 (10.58); 8.923 (6.61); 8.5285 (5.54); 8.5241 (5.26); 8.349 (3.58); 8.3427 (3.46); 8.3281
(3.76); 8.3217 (3.7); 8.3131 (0.43); 7.8689 (7.75); 7.863 (7.69); 7.651 (6.34); 7.63 (5.99); 6.6992 (6.72); 6.6934
(6.66); 5.6307 (16); 3.3468 (382.22); 3.3421 (479.14); 2.9953 (0.52); 2.7123 (0.37); 2.6811 (0.43); 2.6768 (0.89);
2.6723 (1.23); 2.6678 (0.89); 2.6632 (0.41); 2.5425 (98.83); 2.5256 (3.55); 2.5207 (5.28); 2.5122 (69.51); 2.5077
(139.53); 2.5031 (185.38); 2.4985 (134.63); 2.4941 (63.84); 2.3687 (0.38); 2.3389 (0.42); 2.3344 (0.88); 2.3299
(1.22); 2.3253 (0.86); 2.321 (0.39); −0.0002 (2.89)
Compound No. I-1-116, solvent: [DMSO], spectrometer: 399.95 MHz
11.105 (5.35); 9.0903 (5.07); 9.086 (4.95); 8.9348 (5.34); 8.9323 (5.23); 8.7152 (4.17); 8.7112 (4.43); 8.7032
(4.37); 8.6991 (4.27); 8.5324 (5.51); 8.5287 (5.27); 8.3165 (0.7); 8.31 (2.11); 8.3051 (2.97); 8.3002 (2.08); 8.29
(2.22); 8.2849 (3.05); 8.2802 (2.02); 7.863 (7.71); 7.8572 (7.62); 7.5162 (2.9); 7.5145 (3.01); 7.5042 (2.87);
7.5024 (2.95); 7.4963 (2.82); 7.4945 (2.85); 7.4843 (2.68); 7.4825 (2.7); 6.7093 (6.47); 6.7035 (6.38); 5.6316 (16);
3.5679 (2.2); 3.3227 (88.64); 2.6798 (0.5); 2.6754 (1.04); 2.6707 (1.44); 2.6662 (1.04); 2.6615 (0.51); 2.5241
(5.38); 2.5192 (8.57); 2.5107 (83.22); 2.5063 (164.45); 2.5017 (214.4); 2.4971 (153.48); 2.4926 (72.98); 2.3375
(0.49); 2.333 (1.04); 2.3284 (1.4); 2.3239 (1.02); 2.3194 (0.49); −0.0002 (5.74)
Compound No. I-1-128, solvent: [DMSO], spectrometer: 399.95 MHz
11.0842 (3.67); 8.7901 (6.31); 8.778 (6.38); 8.6469 (3.9); 8.3526 (2.19); 8.3363 (2.22); 7.8443 (2.13); 7.8243
(2.34); 7.5255 (1.69); 7.5069 (2.39); 7.4897 (1.47); 7.4537 (1.78); 7.4415 (3.26); 7.4294 (1.7); 6.5002 (4.82);
5.4266 (9.94); 3.3265 (12.95); 2.6717 (0.44); 2.5026 (64.24); 2.3131 (16); −0.0002 (3.96)
Compound No. I-1-68, solvent: [DMSO], spectrometer: 399.95 MHz
11.0958 (5.44); 9.0804 (5.51); 9.0761 (5.44); 8.7094 (4.18); 8.7054 (4.47); 8.6975 (4.4); 8.6934 (4.28); 8.3025
(2.24); 8.2978 (3.23); 8.2928 (2.26); 8.2826 (2.43); 8.2777 (3.37); 8.2729 (2.24); 7.7956 (6.29); 7.7901 (6.22);
7.506 (3.87); 7.4939 (3.41); 7.488 (4.55); 7.4859 (4.5); 7.4738 (3.67); 7.4679 (3.72); 7.4509 (1.85); 7.4469 (2.18);
7.4301 (0.95); 7.1832 (0.66); 7.1791 (0.91); 7.1688 (5.36); 7.1584 (1.31); 7.1489 (8.15); 7.1397 (1.32); 7.1288
(4.54); 7.118 (0.75); 6.6523 (6.87); 6.6466 (6.74); 5.3498 (16); 3.5684 (2.4); 3.3262 (12.24); 2.6716 (0.42); 2.5113
(28.02); 2.507 (52.81); 2.5026 (67.34); 2.498 (49.13); 2.4937 (24.41); 2.3338 (0.34); 2.3293 (0.44); 2.3248 (0.33);
0.0079 (0.38); −0.0002 (7.87); −0.0084 (0.33)
Compound No. I-1-65, solvent: [DMSO], spectrometer: 399.95 MHz
11.2029 (3.03); 9.1267 (3.24); 9.1222 (3.11); 8.7373 (2.62); 8.7332 (2.83); 8.7253 (2.78); 8.7212 (2.76); 8.3453
(1.29); 8.3402 (1.9); 8.3355 (1.31); 8.3254 (1.4); 8.3202 (2.01); 8.3156 (1.32); 7.8435 (4.6); 7.8376 (4.56); 7.5419
(1.84); 7.5402 (1.98); 7.5298 (1.8); 7.5282 (1.95); 7.522 (1.78); 7.5203 (1.89); 7.51 (1.71); 7.5082 (1.82); 6.7181
(4); 6.7122 (3.94); 4.9978 (0.48); 4.9813 (0.41); 4.969 (0.55); 4.9652 (0.46); 4.953 (0.91); 4.941 (0.71); 4.9377
(0.75); 4.9265 (1.32); 4.9025 (3); 4.8871 (3.88); 4.8769 (2.1); 4.8644 (0.74); 4.8523 (0.43); 4.8079 (0.71); 4.7833
(2.88); 4.7711 (3.54); 4.7677 (3.15); 4.7594 (2.3); 4.7468 (0.74); 4.7346 (0.41); 3.5679 (0.59); 3.3237 (23.24);
2.5243 (1); 2.5108 (16.85); 2.5064 (33.75); 2.5018 (44.33); 2.4972 (31.57); 2.4927 (14.9); 2.0859 (16); 1.9889
(0.83); 1.1744 (0.45); 0.008 (0.59); −0.0002 (16.34); −0.0086 (0.47)
Compound No. I-1-10, solvent: [DMSO], spectrometer: 399.95 MHz
11.2613 (10.17); 9.1245 (10.86); 9.12 (10.69); 8.7412 (8.57); 8.7372 (9.15); 8.7292 (9.01); 8.7251 (8.87); 8.3457
(4.57); 8.3408 (6.65); 8.3359 (4.59); 8.3258 (4.88); 8.3208 (6.94); 8.316 (4.58); 8.0063 (0.42); 7.9997 (0.43);
7.8191 (15.1); 7.8131 (15.08); 7.5443 (6.21); 7.5429 (6.33); 7.5322 (6.15); 7.5308 (6.26); 7.5244 (6.01); 7.523
(6.08); 7.5124 (5.74); 7.5109 (5.8); 6.7806 (14.07); 6.7746 (13.85); 6.6696 (0.49); 6.6632 (0.5); 5.7577 (0.36);
5.3111 (0.4); 5.2887 (0.43); 5.1141 (4.71); 5.0914 (15.23); 5.0686 (16); 5.0457 (5.43); 3.3215 (92.5); 2.6794
(0.64); 2.6751 (1.32); 2.6706 (1.81); 2.6661 (1.32); 2.6617 (0.62); 2.5238 (6.97); 2.5189 (10.67); 2.5105 (102.78);
2.5061 (203.49); 2.5015 (266.6); 2.497 (191.68); 2.4925 (92.04); 2.3373 (0.62); 2.3328 (1.29); 2.3283 (1.77);
2.3237 (1.28); 2.3193 (0.61); 2.1388 (0.34); 1.2333 (0.39); 0.1459 (0.79); 0.0079 (6.68); −0.0003 (182.05); −0.0086
(6.37); −0.1497 (0.82)
Compound No. I-1-2: mass (m/z): 261.1 (M + H)⁺; 1H NMR (CD₃CN) 1.10-1.16 (t, 3H), 2.33 (s, 3H),
3.49-3.54 (q, 2H), 5.29 (s, 2H), 6.54 (s, 1H), 7.41-7.45 (m, 1H), 8.17-8.20 (m, 1H), 8.70-8.71 (m, 1H), 8.91 (br. s, 1H),
9.05 (s, 1H).
Compound No. I-1-13, solvent: [DMSO], spectrometer: 399.95 MHz
11.2004 (9.57); 9.1215 (10.71); 9.1174 (10.62); 9.116 (9.9); 8.7389 (9.46); 8.7348 (10.03); 8.7269 (9.98); 8.7227
(9.7); 8.3403 (4.84); 8.3356 (6.51); 8.3304 (4.84); 8.3204 (5.17); 8.3152 (6.75); 8.3105 (4.75); 7.9274 (0.42);
7.921 (0.42); 7.7939 (0.36); 7.7689 (0.47); 7.763 (0.71); 7.7526 (16); 7.7467 (15.94); 7.5439 (6.8); 7.5419 (6.78);
7.5319 (6.64); 7.5299 (6.59); 7.524 (6.54); 7.522 (6.42); 7.512 (6.36); 7.51 (6.21); 6.7215 (14.18); 6.7157 (13.94);
6.6009 (0.51); 6.5946 (0.49); 6.4839 (1.78); 6.4745 (3.59); 6.4652 (1.56); 6.3466 (3.57); 6.3372 (7.81); 6.3278
(3.43); 6.275 (0.37); 6.269 (0.41); 6.2092 (1.73); 6.1998 (3.92); 6.1905 (1.78); 5.6649 (0.32); 4.997 (0.35); 4.6165
(5.54); 4.6072 (5.92); 4.5788 (12.33); 4.5694 (11.91); 4.5578 (0.63); 4.541 (6.32); 4.5317 (5.85); 4.113 (0.4);
4.0998 (1.17); 4.0867 (1.19); 4.0736 (0.42); 3.3224 (164.26); 3.1746 (5.69); 3.1615 (5.49); 2.6796 (0.62); 2.6752
(1.34); 2.6706 (1.87); 2.666 (1.35); 2.6613 (0.62); 2.5515 (0.45); 2.524 (6.02); 2.5193 (8.91); 2.5106 (103.82);
2.5061 (211.76); 2.5015 (279.15); 2.4969 (198.41); 2.4923 (93.16); 2.3373 (0.61); 2.3329 (1.33); 2.3283 (1.85);
2.3237 (1.32); 2.3191 (0.62); 1.9799 (0.87); 1.2982 (0.48); 1.2584 (0.74); 1.2337 (1.11); 1.2249 (0.45); 1.1336
(0.44); 1.1161 (0.89); 1.0985 (0.45); 0.146 (0.41); 0.008 (3.45); −0.0002 (110.9); −0.0086 (3.51); −0.1497 (0.43)
Compound No. I-1-48, solvent: [CD3CN], spectrometer: 399.95 MHz
9.4678 (3.5); 9.1159 (11.57); 9.1119 (11.83); 8.7531 (8.46); 8.7491 (8.63); 8.741 (8.89); 8.7371 (8.3); 8.5255
(0.82); 8.5177 (0.86); 8.5139 (0.84); 8.4673 (9.23); 8.4636 (9.13); 8.4557 (9.5); 8.4521 (8.87); 8.3522 (0.41);
8.2789 (5.68); 8.2746 (7.42); 8.2692 (5.11); 8.259 (6.14); 8.2535 (8.68); 8.2492 (5.65); 8.183 (0.35); 8.1155 (16);
8.1089 (15.75); 8.0367 (9.81); 8.0332 (9.12); 8.0166 (10.56); 8.013 (9.66); 7.9292 (0.56); 7.5173 (0.98); 7.5017
(7.24); 7.5 (6.38); 7.4895 (7.45); 7.4878 (6.84); 7.4817 (7.3); 7.4696 (6.67); 7.4242 (9.49); 7.4125 (9.51); 7.404
(9.12); 7.3925 (8.47); 7.3157 (0.54); 7.2973 (0.62); 7.2836 (0.76); 7.2637 (1.4); 7.2503 (1.46); 7.1911 (1.39);
7.1728 (1.04); 7.157 (0.91); 7.1353 (0.59); 7.0322 (14.15); 7.0257 (13.66); 6.9733 (0.67); 6.8915 (1.55); 6.8846
(1.47); 5.9789 (0.39); 5.9714 (0.37); 5.4473 (2.42); 5.2091 (0.32); 3.6001 (4.8); 2.6305 (0.67); 2.463 (1.52);
2.4582 (1.19); 2.3641 (0.6); 2.2216 (1.31); 2.1462 (455.25); 2.1198 (5.12); 2.1133 (4.96); 2.1071 (5.09); 2.101
(3.55); 2.095 (2.01); 1.9637 (33.68); 1.9575 (53.42); 1.9519 (256.12); 1.9457 (459.7); 1.9396 (599.75); 1.9335
(416.36); 1.9274 (217.09); 1.7803 (1.82); 1.7741 (2.95); 1.7681 (3.81); 1.7618 (2.69); 1.7557 (1.59); 1.629 (1.03);
1.6178 (1.39); 1.5454 (0.59); 1.53 (0.56); 1.3875 (10.31); 1.2712 (10.53); 1.2553 (3.32); 1.2377 (2.63); 1.1903
(0.88); 1.1313 (0.83); 1.1139 (1.08); 1.0964 (0.9); 0.9339 (0.51); 0.8818 (2.77); 0.8767 (2.35); 0.8578 (2.77);
0.8393 (1.69); 0.2256 (2.42); 0.1462 (1.26); −0.0002 (259.77); −0.0079 (14.1); −0.1492 (1.21)
Compound No. I-1-23 (Synthesis Example 13): 1H NMR (DMSO-D₆) 2.66 (s, 3H), 6.83 (s, 1H), 7.41-7.43 (m,
1H), 7.49-7.53 (m, 1H), 8.35-8.38 (m, 1H), 8.72-8.74 (m, 1H), 8.84-8.85 (m, 2H), 9.17 (s, 1H), 11.26 (s,
1H).
Compound No. I-1-16, solvent: [DMSO], spectrometer: 399.95 MHz
8.5322 (1.69); 8.5228 (1.74); 8.4772 (1.46); 8.3155 (0.5); 7.7336 (0.94); 7.7176 (1); 7.3603 (1.02); 7.3476 (1.25);
7.3306 (0.91); 6.4401 (0.41); 3.9021 (2.23); 3.6664 (2.4); 3.5739 (16); 3.5243 (0.37); 3.3306 (106.01); 3.2854
(41.42); 2.6752 (0.61); 2.6709 (0.84); 2.6662 (0.59); 2.541 (0.5); 2.5241 (2.12); 2.5107 (54.82); 2.5064 (108.01);
2.5018 (139.17); 2.4973 (99.74); 2.4929 (48.2); 2.3332 (0.63); 2.3286 (0.84); 2.324 (0.62); 2.2541 (1.72); 2.1307
(12.56); 1.235 (0.84); 0.008 (0.41); −0.0002 (12.78); −0.0085 (0.41)
Compound No. I-1-55, solvent: [DMSO], spectrometer: 399.95 MHz
11.4245 (2.93); 9.1654 (2.05); 9.1618 (2.03); 9.1126 (3.96); 9.1092 (3.91); 8.7665 (1.42); 8.7579 (1.43); 8.584
(3.21); 8.5775 (4.94); 8.5583 (3.39); 8.5547 (3.37); 8.5519 (2.3); 8.5483 (2.06); 8.3827 (1.21); 8.3781 (1.63);
8.3729 (1.19); 8.3628 (1.3); 8.3578 (1.71); 8.3529 (1.21); 8.315 (0.37); 7.572 (1.29); 7.5598 (1.31); 7.5531 (1.29);
7.5412 (1.21); 6.8889 (4.09); 3.9023 (2.09); 3.3269 (107.68); 2.671 (16); 2.6696 (15.46); 2.5416 (0.68); 2.5248
(2.16); 2.5115 (45.57); 2.507 (90.92); 2.5025 (117.85); 2.4979 (82.89); 2.4933 (38.32); 2.3338 (0.52); 2.3293
(0.69); 2.3246 (0.5); 1.2303 (0.87); 0.008 (0.42); −0.0002 (12.29); −0.0085 (0.32)
Compound No. I-1-51, solvent: [DMSO], spectrometer: 399.95 MHz
11.0667 (2.54); 9.1155 (1.81); 8.7289 (1.33); 8.7192 (1.34); 8.5513 (1.7); 8.3237 (1.45); 8.3194 (1.04); 8.3162
(0.85); 8.3085 (1.1); 8.3039 (1.49); 7.5298 (1.23); 7.518 (1.28); 7.5099 (1.23); 7.4979 (1.13); 7.0918 (2.86);
7.0791 (2.76); 6.5843 (4.12); 5.3112 (7.61); 3.9023 (2.39); 3.3341 (18); 3.1686 (1.07); 2.6756 (0.61); 2.6711
(0.78); 2.6667 (0.56); 2.5414 (0.45); 2.5244 (2.29); 2.5111 (52.85); 2.5067 (103.65); 2.5021 (133.21); 2.4976
(95.08); 2.4931 (45.4); 2.3335 (0.59); 2.3289 (0.8); 2.3243 (0.58); 2.2615 (16); 2.2365 (0.43); 0.0079 (0.44); −0.0002
(12.74); −0.0085 (0.37)
Compound No. I-1-33, solvent: [DMSO], spectrometer: 399.95 MHz
11.0522 (2.23); 9.1158 (1.67); 8.7321 (1.21); 8.7219 (1.2); 8.3327 (0.97); 8.3279 (1.37); 8.3232 (0.91); 8.3128
(1.04); 8.3081 (1.43); 8.3033 (0.92); 7.5362 (1.15); 7.5242 (1.2); 7.5166 (1.16); 7.5044 (1.06); 6.4815 (3.86);
4.3253 (2.28); 4.3107 (5.04); 4.2963 (2.63); 3.9809 (2.67); 3.9665 (5.13); 3.9519 (2.27); 3.9023 (1.48); 3.3325
(63.82); 3.1685 (0.33); 2.6756 (0.43); 2.6713 (0.59); 2.6665 (0.43); 2.5244 (1.48); 2.511 (38.98); 2.5066 (77.58);
2.5021 (100.32); 2.4975 (71.24); 2.493 (33.84); 2.4654 (0.36); 2.3332 (0.49); 2.3287 (0.71); 2.3243 (0.59); 2.3063
(16); −0.0002 (9.05)
Compound No. I-1-5, solvent: [DMSO], spectrometer: 399.95 MHz
11.036 (2.16); 9.1258 (1.21); 8.7388 (0.93); 8.7315 (0.92); 8.3344 (0.91); 8.33 (1.33); 8.3255 (0.9); 8.3151 (1.14);
8.3101 (1.37); 8.3056 (0.87); 7.5401 (0.97); 7.5282 (1.04); 7.5206 (1.01); 7.5083 (0.89); 6.4831 (3.77); 4.7996
(1.26); 4.7879 (2.28); 4.7759 (1.37); 4.6815 (1.24); 4.6698 (2.29); 4.6577 (1.4); 4.3457 (1.27); 4.3338 (2.14);
4.322 (1.17); 4.278 (1.33); 4.2661 (2.15); 4.2544 (1.1); 3.9023 (1.97); 3.337 (52.28); 2.6762 (0.32); 2.6714 (0.47);
2.6668 (0.32); 2.5417 (0.36); 2.5248 (1.46); 2.5114 (31.22); 2.5069 (61.27); 2.5024 (78.95); 2.4978 (56.38);
2.4933 (26.93); 2.3336 (0.38); 2.3292 (0.49); 2.3246 (0.37); 2.2754 (16); −0.0002 (8.18)
Compound No. I-1-1, solvent: [DMSO], spectrometer: 600.13 MHz
10.990 (3.4); 9.102 (3.2); 9.099 (3.2); 8.794 (6.8); 8.786 (6.8); 8.714 (2.2); 8.712 (2.3); 8.706 (2.3); 8.704 (2.2);
8.313 (1.8); 8.300 (1.9); 7.513 (1.6); 7.505 (1.8); 7.500 (1.7); 7.492 (1.5); 7.453 (1.8); 7.444 (3.4); 7.436 (1.7);
6.532 (4.3); 5.431 (10.3); 3.346 (5.1); 2.507 (9.9); 2.324 (16.0)
Compound No. I-1-4, solvent: [DMSO], spectrometer: 399.95 MHz
11.0907 (2.57); 9.1104 (2.62); 9.106 (2.55); 8.7307 (1.9); 8.7268 (2); 8.7188 (1.98); 8.7149 (1.9); 8.3304 (1.16);
8.3257 (1.6); 8.3206 (1.12); 8.3148 (0.61); 8.3104 (1.26); 8.3054 (1.66); 8.3006 (1.1); 7.5346 (1.48); 7.533 (1.48);
7.5224 (1.49); 7.5209 (1.5); 7.5147 (1.46); 7.513 (1.42); 7.5026 (1.36); 7.501 (1.32); 6.5409 (4.18); 6.4718 (0.38);
6.4625 (0.76); 6.4533 (0.34); 6.3347 (0.78); 6.3253 (1.65); 6.3161 (0.74); 6.1976 (0.38); 6.188 (0.81); 6.1789
(0.39); 4.5348 (1.24); 4.5257 (1.31); 4.497 (2.71); 4.4877 (2.61); 4.4592 (1.39); 4.4498 (1.27); 3.9022 (1.26);
3.3281 (157.45); 2.6753 (0.54); 2.6708 (0.71); 2.6663 (0.52); 2.5411 (0.42); 2.5106 (47.54); 2.5063 (90.79);
2.5018 (115.85); 2.4972 (82.7); 2.4928 (39.52); 2.3332 (0.58); 2.3285 (0.79); 2.3236 (0.69); 2.2985 (16); 0.0079
(1.02); −0.0002 (21.85); −0.0085 (0.77)
Compound No. I-1-38, solvent: [DMSO], spectrometer: 399.95 MHz
10.9825 (2.06); 9.111 (1.89); 8.7207 (1.37); 8.7121 (1.36); 8.3266 (0.94); 8.3219 (1.34); 8.3169 (0.94); 8.3067
(1.03); 8.3019 (1.4); 8.2972 (0.91); 7.5278 (1.19); 7.5157 (1.21); 7.5079 (1.2); 7.4958 (1.1); 6.4383 (3.86); 4.0198
(1.43); 4.0018 (4.71); 3.9837 (4.76); 3.9657 (1.48); 3.9023 (1.26); 3.3269 (73.21); 2.6756 (0.34); 2.6711 (0.46);
2.6665 (0.33); 2.5245 (1.31); 2.5111 (32.01); 2.5066 (62.9); 2.5021 (80.69); 2.4975 (57.17); 2.493 (26.9); 2.3334
(0.38); 2.3287 (0.51); 2.3242 (0.37); 2.269 (16); 1.3213 (5.07); 1.3033 (11.2); 1.2852 (4.94); −0.0002 (7.72)
Compound No. I-1-15, solvent: [DMSO], spectrometer: 399.95 MHz
11.0372 (2.51); 9.1006 (1.99); 8.7207 (1.41); 8.7175 (1.48); 8.7089 (1.47); 8.508 (1.22); 8.4983 (1.23); 8.4588
(1.68); 8.3182 (1.04); 8.3144 (1.63); 8.3086 (0.97); 8.2984 (1.1); 8.2935 (1.5); 8.2887 (0.97); 7.5521 (1.21);
7.5324 (1.57); 7.5217 (1.38); 7.5086 (1.32); 7.5017 (1.27); 7.4897 (1.18); 7.4006 (1.23); 7.3888 (1.27); 7.3812
(1.06); 7.369 (0.92); 6.5412 (4.12); 5.2861 (8.02); 3.9022 (3); 3.331 (148.75); 3.1687 (0.64); 2.6759 (0.49); 2.6714
(0.67); 2.6668 (0.49); 2.5415 (0.37); 2.5246 (2.01); 2.5114 (45.24); 2.5069 (88.65); 2.5023 (114.2); 2.4977
(80.72); 2.4932 (37.85); 2.3335 (0.59); 2.329 (0.89); 2.3242 (0.87); 2.3125 (16); 0.0079 (0.39); −0.0002 (10.47)
Compound No. I-1-14, solvent: [DMSO], spectrometer: 399.95 MHz
11.0908 (2.63); 9.1089 (2.1); 8.7242 (1.55); 8.7151 (1.52); 8.3265 (1.07); 8.3218 (1.57); 8.3169 (1.12); 8.3065
(1.17); 8.3019 (1.61); 8.2972 (1.03); 7.7865 (3.78); 7.7784 (4.51); 7.7067 (4.83); 7.6986 (3.88); 7.5272 (1.42);
7.5147 (1.46); 7.5071 (1.39); 7.495 (1.31); 6.5593 (4.12); 5.5694 (10.83); 3.902 (2.03); 3.3251 (92.11); 2.6755
(0.59); 2.6711 (0.82); 2.6667 (0.59); 2.5414 (0.52); 2.5243 (2.38); 2.511 (53.83); 2.5066 (106.69); 2.502 (137.88);
2.4974 (97.26); 2.4929 (45.4); 2.3388 (16); 2.329 (1.45); 2.3244 (0.85); 1.2346 (0.56); 0.0079 (0.51); −0.0002
(14.27); −0.0085 (0.42)
Compound No. I-1-44, solvent: [DMSO], spectrometer: 399.95 MHz
11.3077 (3.35); 9.1414 (2.45); 9.1368 (2.41); 9.0294 (2.55); 9.0275 (2.92); 9.0244 (2.91); 8.8361 (3.14); 8.832
(2.96); 8.7539 (1.68); 8.7504 (1.78); 8.7421 (1.74); 8.7385 (1.69); 8.3608 (1.19); 8.3558 (1.66); 8.351 (1.17);
8.3408 (1.27); 8.3357 (1.72); 8.331 (1.13); 8.3153 (0.36); 7.5555 (1.45); 7.5435 (1.46); 7.5367 (1.42); 7.5234
(1.33); 6.8349 (4.36); 3.9022 (2.26); 3.3238 (106.68); 2.6754 (0.64); 2.6708 (0.89); 2.6663 (0.66); 2.541 (0.8);
2.5239 (3.19); 2.5107 (56.29); 2.5063 (109.93); 2.5018 (141.79); 2.4972 (100.86); 2.4927 (47.64); 2.333 (0.65);
2.3286 (0.86); 2.3239 (0.67); 2.3192 (0.41); 2.297 (16); 1.2983 (0.39); 1.2586 (0.53); 1.2327 (0.45); −0.0002 (7.49)
Compound No. I-1-34, solvent: [DMSO], spectrometer: 399.95 MHz
11.2383 (2.69); 9.1427 (1.93); 8.7474 (1.38); 8.7384 (1.36); 8.6105 (2.67); 8.6066 (2.86); 8.5988 (2.83); 8.5949
(2.78); 8.3592 (1.08); 8.3544 (1.52); 8.3495 (1.05); 8.3392 (1.15); 8.3342 (1.57); 8.3295 (1.05); 8.3153 (0.34);
8.286 (2.76); 8.2821 (2.78); 8.2658 (3.05); 8.2619 (2.84); 7.6715 (2.98); 7.6598 (2.82); 7.6513 (2.71); 7.6396
(2.69); 7.5532 (1.26); 7.5406 (1.27); 7.5336 (1.24); 7.5214 (1.16); 6.7727 (4.14); 3.9023 (2.38); 3.3258 (88.39);
2.6757 (0.56); 2.6711 (0.74); 2.6665 (0.55); 2.5414 (0.58); 2.5244 (2.47); 2.5111 (48.22); 2.5066 (94.42); 2.5021
(121.68); 2.4975 (85.81); 2.4929 (39.88); 2.3334 (0.48); 2.3288 (0.7); 2.3243 (0.47); 2.2048 (16); 2.2036 (15.87);
1.2829 (0.39); 1.2347 (0.5); 0.8758 (0.64); 0.8626 (0.46); 0.8569 (0.39); 0.008 (0.42); −0.0002 (11.59)
Compound No. I-1-45, solvent: [DMSO], spectrometer: 399.95 MHz
11.4154 (3.59); 9.1646 (2.46); 9.1602 (2.38); 8.886 (2.48); 8.8843 (2.46); 8.8803 (2.42); 8.7712 (1.66); 8.7676
(1.72); 8.7593 (1.74); 8.7557 (1.64); 8.3948 (1.52); 8.3885 (1.53); 8.3798 (1.46); 8.3738 (2.86); 8.3665 (1.87);
8.36 (1.49); 8.3552 (1.85); 8.3501 (1.24); 8.3154 (0.34); 8.0268 (2.68); 8.0049 (2.42); 7.5742 (1.46); 7.5616
(1.48); 7.5543 (1.43); 7.5423 (1.37); 6.887 (4.52); 3.9025 (1.8); 3.3257 (99.06); 2.7266 (16); 2.6761 (0.57); 2.6715
(0.77); 2.667 (0.57); 2.5418 (0.53); 2.5248 (2.29); 2.5115 (50.31); 2.5071 (98.93); 2.5026 (127.7); 2.498 (90.93);
2.4935 (43.5); 2.3338 (0.57); 2.3293 (0.76); 2.3247 (0.55); 1.3514 (0.34); 1.2982 (0.61); 1.2589 (0.86); 1.2313
(0.6); 0.0079 (0.48); −0.0002 (12.55); −0.0085 (0.44)
Compound No. I-1-43, solvent: [DMSO], spectrometer: 399.95 MHz
11.0618 (6.27); 9.1012 (4.76); 8.7281 (3.38); 8.7195 (3.37); 8.3152 (3.28); 8.3103 (3.61); 8.3054 (2.51); 8.2953
(2.79); 8.2902 (3.72); 8.2855 (2.45); 7.5334 (3.06); 7.5221 (3.12); 7.5138 (2.98); 7.5025 (2.76); 6.3135 (12.89);
4.9991 (16); 4.9928 (15.69); 3.9022 (6.47); 3.4126 (5.02); 3.4065 (11.29); 3.4003 (4.65); 3.3712 (0.46); 3.3243
(165.08); 3.1749 (0.33); 3.1619 (0.37); 2.6757 (1.25); 2.671 (1.68); 2.6664 (1.23); 2.6619 (0.62); 2.5681 (0.4);
2.5636 (0.51); 2.5413 (1.29); 2.5242 (5.35); 2.5109 (109.53); 2.5065 (213.36); 2.5019 (273); 2.4973 (191.54);
2.4928 (89.2); 2.3377 (0.54); 2.3332 (1.19); 2.3287 (1.59); 2.3241 (1.17); 1.9857 (0.79); 1.9728 (1.67); 1.9645
(1.89); 1.9522 (3.44); 1.9399 (2.01); 1.9317 (1.8); 1.9189 (0.88); 1.3363 (0.45); 1.2982 (0.92); 1.2587 (1.35);
1.2354 (1.09); 1.225 (0.74); 1.0196 (2.45); 1.0088 (6.64); 1.0033 (6.89); 0.9988 (3.53); 0.993 (3.67); 0.9879
(6.48); 0.9824 (6.65); 0.9723 (2.57); 0.9575 (0.41); 0.9355 (0.36); 0.8783 (0.41); 0.8556 (0.35); 0.6928 (2.85);
0.6826 (7.26); 0.6772 (7.23); 0.6701 (7); 0.6648 (7.34); 0.654 (2.26); 0.008 (1); −0.0002 (28.42); −0.0087 (0.78)
Compound No. I-1-40, solvent: [DMSO], spectrometer: 399.95 MHz
11.0644 (3.41); 9.116 (2.49); 8.7334 (1.77); 8.7244 (1.75); 8.3295 (1.35); 8.3246 (1.95); 8.3199 (1.36); 8.3152
(0.73); 8.3096 (1.48); 8.3045 (2.02); 8.3 (1.31); 7.5393 (1.63); 7.5272 (1.65); 7.5194 (1.61); 7.5074 (1.47); 6.5219
(6.07); 5.0353 (0.43); 5.0289 (0.42); 4.9031 (8.57); 4.8968 (8.46); 3.9022 (2.39); 3.4991 (0.35); 3.4128 (2.83);
3.4067 (6.51); 3.4005 (2.64); 3.3249 (89.02); 2.8388 (0.34); 2.8198 (0.33); 2.7138 (1.64); 2.6947 (5.09); 2.6758
(5.74); 2.6664 (0.87); 2.6572 (1.75); 2.6287 (0.49); 2.6096 (0.5); 2.5414 (0.59); 2.5243 (2.43); 2.5109 (52.34);
2.5065 (102.48); 2.502 (131.97); 2.4974 (93.85); 2.4929 (44.4); 2.4098 (0.38); 2.3333 (0.59); 2.3287 (0.79);
2.3242 (0.56); 1.2981 (0.46); 1.2583 (1.03); 1.251 (7.43); 1.2427 (1.44); 1.2322 (16); 1.2133 (7.22); 1.1974 (0.96);
1.1931 (0.37); 1.1783 (0.49); 0.0079 (0.42); −0.0002 (12.46); −0.0086 (0.38)
Compound No. I-1-54, solvent: [DMSO], spectrometer: 399.95 MHz
11.1808 (5.05); 9.1292 (3.95); 8.7395 (2.81); 8.7301 (2.77); 8.3444 (2.25); 8.3396 (3.24); 8.3348 (2.22); 8.3245
(2.42); 8.3195 (3.43); 8.3149 (2.66); 7.7397 (8.3); 7.7339 (8.23); 7.5451 (2.7); 7.5331 (2.75); 7.5257 (2.68);
7.5137 (2.47); 6.6748 (7.23); 6.6691 (7.09); 4.9883 (16); 4.982 (15.93); 3.9021 (2.7); 3.4869 (4.61); 3.4806
(10.07); 3.4743 (4.34); 3.3254 (110.21); 3.1751 (1.31); 3.1622 (1.31); 2.6756 (0.78); 2.6712 (1.05); 2.6666 (0.77);
2.5413 (0.81); 2.5243 (2.85); 2.5109 (66.35); 2.5066 (132.31); 2.502 (171.79); 2.4975 (123.01); 2.4931 (58.76);
2.3332 (0.73); 2.3289 (0.99); 2.3244 (0.74); 0.0079 (0.53); −0.0002 (16.36); −0.0084 (0.53)
Compound No. I-1-79 (Synthesis Example 7), solvent: [DMSO], spectrometer: 399.95 MHz
11.4076 (2.83); 9.6377 (2.26); 9.6348 (2.69); 9.6323 (2.61); 9.6293 (2.16); 9.4434 (2.13); 9.4407 (2.08); 9.4302
(2.21); 9.4274 (2.11); 8.1351 (2.08); 8.1292 (2.08); 8.1218 (2.06); 8.1159 (1.97); 7.3666 (1.63); 7.3628 (0.71);
7.3492 (4.47); 7.3303 (3.48); 7.2953 (1.78); 7.2822 (0.71); 7.277 (2.17); 7.2708 (0.51); 7.2587 (0.65); 7.1551
(3.8); 7.1374 (3.23); 6.552 (4.86); 5.25 (8.33); 3.9022 (3.49); 3.3257 (84.46); 3.1676 (0.35); 2.6755 (0.53); 2.671
(0.7); 2.6664 (0.5); 2.5415 (0.54); 2.5243 (2.04); 2.5109 (45.85); 2.5065 (89.88); 2.502 (115.63); 2.4974 (82.09);
2.4929 (38.81); 2.3332 (0.51); 2.3288 (0.69); 2.3241 (0.49); 2.2757 (16); 1.2349 (0.37); 0.0081 (0.38); −0.0002
(11); −0.0086 (0.33)
Compound No. I-1-6: mass (m/z): 294.1 (M + H)⁺; 1H NMR (CD₃CN) 2.28 (s, 3H), 5.27 (s, 2H), 6.54 (s, 1H),
6.98-7.00 (m, 1H), 7.21-7.24 (m, 1H), 7.40-7.44 (m, 1H), 7.67-7.71 (m, 1H), 8.17-8.20 (m, 1H), 8.50-8.51 (m,
1H), 8.69-8.70 (m, 1H), 8.94 (br. s, 1H), 9.04 (s, 1H).
Compound No. I-1-66, solvent: [DMSO], spectrometer: 399.95 MHz
11.2631 (2.78); 9.317 (6.75); 9.2585 (12.79); 8.315 (0.33); 7.3694 (1.11); 7.3665 (1.77); 7.3625 (0.8); 7.349
(4.74); 7.3456 (2.63); 7.3302 (3.8); 7.3135 (0.34); 7.2947 (1.92); 7.2915 (1.24); 7.2818 (0.85); 7.2765 (2.34);
7.2703 (0.69); 7.2581 (0.8); 7.155 (3.77); 7.1373 (3.19); 7.1137 (0.38); 7.0944 (0.35); 6.5403 (4.59); 5.2434
(8.43); 5.1764 (0.39); 5.155 (0.46); 3.9021 (4.04); 3.3309 (71.79); 3.3282 (71.38); 2.6758 (0.51); 2.6711 (0.69);
2.6665 (0.49); 2.5413 (0.49); 2.5244 (2.21); 2.5111 (44.79); 2.5067 (87.23); 2.5021 (111.67); 2.4975 (78.96);
2.493 (37.19); 2.3333 (0.49); 2.3288 (0.68); 2.3242 (0.48); 2.2735 (16); 2.204 (0.6); 2.1976 (0.33); 2.1847 (0.9);
1.8664 (0.35); 1.2983 (0.75); 1.2587 (1.06); 0.0079 (0.43); −0.0002 (11.6); −0.0086 (0.35)
Compound No. I-1-56, solvent: [DMSO], spectrometer: 399.95 MHz
11.0446 (5.25); 9.0931 (3.53); 8.7173 (2.57); 8.7078 (2.53); 8.3152 (0.82); 8.3076 (1.94); 8.3031 (2.84); 8.2982
(1.91); 8.2877 (2.08); 8.2829 (2.91); 8.2784 (1.87); 7.5194 (2.43); 7.5077 (2.47); 7.5002 (2.4); 7.4881 (2.23);
7.3701 (3.46); 7.3662 (1.56); 7.3525 (9.31); 7.3493 (5.09); 7.3336 (7.56); 7.3201 (1.94); 7.2961 (3.59); 7.293
(2.42); 7.2832 (1.47); 7.2778 (4.45); 7.2717 (1.13); 7.2595 (1.42); 7.1993 (7.64); 7.1817 (6.15); 6.339 (10.52);
5.3572 (16); 4.3241 (0.49); 4.3086 (0.46); 3.9021 (6.07); 3.3257 (166.84); 3.1752 (0.43); 3.1624 (0.42); 2.6799
(0.44); 2.6754 (0.9); 2.671 (1.22); 2.6664 (0.89); 2.5412 (1.03); 2.5242 (3.83); 2.5109 (81.5); 2.5065 (160.87);
2.5019 (207.93); 2.4973 (147.4); 2.4928 (69.4); 2.3333 (0.89); 2.3286 (1.19); 2.3242 (0.88); 1.9541 (0.63); 1.9416
(1.33); 1.933 (1.53); 1.9207 (2.73); 1.9084 (1.68); 1.9001 (1.41); 1.8875 (0.7); 1.2983 (0.79); 1.2586 (1.16);
1.2351 (0.76); 0.9784 (0.34); 0.9609 (1.97); 0.9501 (5.29); 0.9446 (5.46); 0.9402 (2.92); 0.9344 (2.9); 0.9293
(5.1); 0.9238 (5.35); 0.9138 (2.01); 0.8385 (0.38); 0.8321 (0.33); 0.8262 (0.37); 0.6498 (2.22); 0.6395 (5.82);
0.6343 (5.89); 0.6271 (5.65); 0.6219 (5.86); 0.611 (1.77); 0.008 (0.69); −0.0002 (21.48); −0.0085 (0.62)
Compound No. I-1-58, solvent: [DMSO], spectrometer: 399.95 MHz
11.0606 (3.98); 9.1004 (2.94); 8.7236 (2.12); 8.7141 (2.13); 8.3142 (2.26); 8.308 (1.53); 8.293 (6.09); 8.2877
(5.35); 7.6011 (1.76); 7.5951 (1.7); 7.5805 (3.02); 7.5744 (3); 7.527 (7.62); 7.5154 (2.1); 7.5064 (4.97); 7.4958
(1.74); 7.3915 (0.33); 6.5729 (6.61); 5.3012 (12.17); 3.9022 (1.06); 3.5113 (0.37); 3.4136 (0.34); 3.3956 (0.4);
3.3274 (248.86); 3.1752 (0.6); 3.1624 (0.52); 2.7009 (1.89); 2.6822 (6); 2.6713 (1.99); 2.6635 (6.27); 2.6445
(2.02); 2.6165 (0.32); 2.571 (0.34); 2.5415 (1.07); 2.5243 (4.49); 2.511 (93.98); 2.5068 (177.56); 2.5024 (223.43);
2.4979 (158.87); 2.4938 (76.61); 2.3335 (0.96); 2.3292 (1.23); 2.3245 (0.92); 1.2984 (3.97); 1.2588 (5.41); 1.2351
(2.63); 1.2057 (7.68); 1.1869 (16); 1.1682 (7.19); 1.1309 (0.42); 0.8541 (0.42); 0.0078 (0.63); −0.0002 (17.04); −0.0081
(0.59)
Compound No. I-1-26: mass (m/z): 280.1 (M + H)⁺; 1H NMR (DMSO-D₆) 5.36 (s, 2H), 6.68 (s, 1H),
7.10-7.12 (m, 1H), 7.28-7.31 (m, 1H), 7.46-7.50 (m, 1H), 7.74-7.80 (m, 2H), 8.28-8.31 (m, 1H), 8.52-8.53 (m, 1H),
8.69-8.70 (m, 1H), 9.10 (s, 1H), 10.90 (s, 1H),
Compound No. I-1-72, solvent: [DMSO], spectrometer: 399.95 MHz
11.1127 (2.35); 9.1049 (2.26); 9.1005 (2.23); 8.7211 (1.61); 8.7174 (1.69); 8.7092 (1.68); 8.7055 (1.58); 8.3223
(1.02); 8.3176 (1.48); 8.313 (1.02); 8.3025 (1.09); 8.2976 (1.49); 8.2929 (0.95); 7.8201 (3.67); 7.8145 (3.59);
7.523 (1.34); 7.5111 (1.37); 7.5032 (1.31); 7.4911 (1.23); 7.2525 (1.23); 7.2337 (3.05); 7.2148 (1.99); 7.1144
(1.82); 7.0949 (1.38); 7.0722 (2.88); 7.0521 (1.74); 7.0338 (1.42); 6.6721 (3.21); 6.6666 (3.12); 5.2248 (9.11);
3.902 (1.57); 3.3284 (127.81); 3.1753 (0.52); 3.1625 (0.5); 2.6756 (0.45); 2.671 (0.62); 2.6666 (0.45); 2.5412
(0.4); 2.5243 (1.91); 2.511 (40.97); 2.5066 (79.71); 2.502 (102.01); 2.4975 (72.3); 2.4931 (34.21); 2.3332 (0.46);
2.3288 (0.61); 2.3243 (0.45); 2.2805 (16); 0.008 (0.34); −0.0002 (9.66)
Compound No. I-1-84, solvent: [DMSO], spectrometer: 399.95 MHz
11.1184 (4); 9.1045 (3.67); 9.1001 (3.64); 8.721 (2.58); 8.7174 (2.77); 8.7092 (2.76); 8.7055 (2.62); 8.3217 (1.66);
8.316 (2.57); 8.3122 (1.68); 8.3018 (1.84); 8.2968 (2.51); 8.2922 (1.65); 7.8373 (6.75); 7.8316 (6.69); 7.5217
(2.2); 7.5096 (2.28); 7.5031 (2.2); 7.4911 (2.09); 7.3746 (1.55); 7.371 (2.7); 7.3671 (1.19); 7.3583 (1.98); 7.3539
(7.29); 7.3501 (4.43); 7.3387 (2.93); 7.3354 (6.94); 7.3114 (1.63); 7.3077 (3.32); 7.3042 (2.36); 7.2957 (1.15);
7.2897 (3.88); 7.2825 (0.98); 7.275 (0.82); 7.2714 (1.24); 7.2678 (0.89); 7.2584 (5.94); 7.2547 (7.02); 7.2377 (5);
6.6794 (5.7); 6.6738 (5.6); 5.2753 (16); 3.9021 (3.66); 3.3259 (181.81); 3.174 (0.8); 3.1627 (0.76); 2.7549 (0.54);
2.6754 (0.89); 2.6708 (1.2); 2.6662 (0.89); 2.6619 (0.42); 2.5411 (0.86); 2.5242 (3.57); 2.5108 (77.77); 2.5064
(153.77); 2.5018 (198.62); 2.4972 (141.08); 2.4927 (66.65); 2.3376 (0.42); 2.3331 (0.88); 2.3286 (1.17); 2.324
(0.87); 0.9869 (0.48); 0.0079 (0.78); −0.0002 (20.79); −0.0085 (0.68)
Compound No. I-1-9: mass (m/z): 281.1 (M + H)⁺; 1H NMR (DMSO-D₆) 5.46 (s, 2H), 6.67 (s, 1H), 7.41-7.49 (m,
2H), 7.78 (s, 1H), 8.28-8.31 (m, 1H), 8.61-8.78 (m, 3H), 9.10 (s, 1H), 10.87 (s, 1H).
Compound No. I-1-21, solvent: [DMSO], spectrometer: 399.95 MHz
11.037 (2.51); 9.1001 (1.66); 8.7229 (1.2); 8.7131 (1.19); 8.3154 (1.41); 8.311 (1.58); 8.3036 (3.11); 8.2967
(3.42); 7.6155 (1.31); 7.6094 (1.27); 7.5949 (2.05); 7.5888 (2); 7.5293 (4.09); 7.5091 (3.21); 7.4916 (1.03); 6.5436
(4.09); 5.2895 (8.39); 3.9023 (3.01); 3.3244 (69.56); 2.6759 (0.65); 2.6712 (0.85); 2.6666 (0.62); 2.5414 (0.72);
2.5242 (2.98); 2.5111 (55.19); 2.5067 (106.8); 2.5022 (136.24); 2.4976 (96.59); 2.4932 (45.64); 2.3334 (0.69);
2.3288 (0.97); 2.3241 (0.84); 2.31 (16); 1.2983 (0.83); 1.2587 (1.2); 1.2352 (0.35); 0.008 (0.46); −0.0002 (13.73); −0.0086
(0.4)
Compound No. I-1-49, solvent: [DMSO], spectrometer: 399.95 MHz
11.0029 (1.84); 9.1119 (2.05); 9.1073 (1.98); 8.7123 (1.46); 8.7085 (1.46); 8.7004 (1.51); 8.6966 (1.37); 8.3325
(0.88); 8.3276 (1.26); 8.3229 (0.85); 8.3125 (1); 8.3066 (2.24); 8.3032 (0.91); 7.5137 (1.13); 7.5016 (1.14); 7.4938
(1.11); 7.4818 (1.03); 6.4307 (3.61); 4.504 (0.42); 4.4876 (1.09); 4.4712 (1.49); 4.4548 (1.11); 4.4384 (0.42);
3.8966 (0.77); 3.3294 (112.76); 3.325 (112.9); 2.6771 (0.44); 2.6726 (0.59); 2.6681 (0.42); 2.5427 (0.46); 2.5257
(1.86); 2.5125 (40.15); 2.5081 (77.22); 2.5035 (98.5); 2.499 (69.54); 2.4945 (32.61); 2.3348 (0.46); 2.3303 (0.62);
2.3257 (0.45); 2.2748 (14.07); 1.3736 (16); 1.3572 (15.83); 0.0078 (1.06); −0.0002 (25.49); −0.0085 (0.79)
Compound No. I-1-110, solvent: [DMSO], spectrometer: 399.95 MHz
10.8332 (1.98); 8.8059 (1.92); 8.8003 (1.91); 8.2612 (1.19); 8.2551 (1.16); 8.2394 (1.24); 8.2332 (1.22); 7.1931
(8.25); 7.178 (3.53); 7.1722 (4.02); 7.1567 (0.34); 7.1504 (0.51); 6.8984 (2.08); 6.8766 (2.04); 6.5068 (2.91);
5.2152 (4.91); 3.913 (16); 3.9023 (2.4); 3.8931 (0.39); 3.326 (50.47); 2.676 (0.36); 2.6715 (0.49); 2.667 (0.37);
2.5416 (0.35); 2.5244 (1.58); 2.5112 (33.62); 2.5068 (65.38); 2.5023 (84.13); 2.4978 (60.68); 2.4934 (29.67);
2.3336 (0.38); 2.3289 (0.51); 2.3246 (0.39); 2.2595 (10.18); −0.0002 (8.02)
Compound No. I-1-97, solvent: [DMSO], spectrometer: 399.95 MHz
11.2093 (3.14); 8.8997 (4.1); 8.8944 (4.16); 8.6305 (4.44); 8.6252 (4.2); 8.3154 (0.34); 7.2215 (0.39); 7.1964
(8.21); 7.1842 (4.49); 7.1751 (5.62); 7.1682 (0.8); 7.1595 (0.48); 7.152 (0.82); 6.5224 (4.79); 5.2263 (7.99);
3.9024 (3.64); 3.3274 (128.85); 3.1747 (0.52); 3.1626 (0.51); 2.6759 (0.61); 2.6713 (0.82); 2.6669 (0.59); 2.5415
(0.67); 2.5245 (2.77); 2.5113 (56.37); 2.5068 (110.37); 2.5023 (141.89); 2.4977 (100.95); 2.4932 (47.93); 2.3381
(0.34); 2.3336 (0.62); 2.3292 (0.86); 2.3245 (0.64); 2.2721 (16); 0.0079 (0.53); −0.0002 (14.91); −0.0086 (0.42)
Compound No. I-1-109, solvent: [DMSO], spectrometer: 399.95 MHz
10.9441 (2.67); 8.9981 (2.68); 8.993 (2.6); 8.3158 (0.32); 8.2207 (1.57); 8.2149 (1.52); 8.2004 (1.66); 8.1947
(1.59); 7.3651 (2.61); 7.3449 (2.48); 7.2186 (0.36); 7.1942 (10.41); 7.1802 (5.03); 7.1766 (3.17); 7.1729 (5.88);
7.1655 (0.72); 7.1573 (0.49); 7.1498 (0.73); 6.5183 (4.35); 5.2185 (8.04); 3.9024 (3.14); 3.3293 (30.53); 3.1686
(1.16); 2.6806 (0.33); 2.6754 (0.55); 2.6709 (0.7); 2.6664 (0.51); 2.541 (0.93); 2.5245 (20.21); 2.5108 (49.12);
2.5065 (93.73); 2.5019 (119.35); 2.4974 (85.14); 2.4929 (40.77); 2.3332 (0.53); 2.3287 (0.7); 2.3242 (0.51);
2.2628 (16); 0.0079 (0.53); −0.0002 (13.22); −0.0085 (0.41)
Compound No. I-1-11: mass (m/z): 242.1 (M + H)⁺; 1H NMR (DMSO-D₆) 2.33 (s, 3H), 5.30 (s, 2H), 6.55 (s, 1H),
7.39-7.51 (m, 1H), 8.29-8.32 (m, 1H), 8.70-8.72 (m, 1H), 9.11 (s, 1H), 10.90 (s, 1H).
Compound No. I-1-83, solvent: [DMSO], spectrometer: 399.95 MHz
11.1519 (2.97); 9.0715 (2.85); 9.0674 (2.79); 8.8691 (3.07); 8.8639 (3.04); 8.5627 (2.08); 8.5578 (3.26); 8.5529
(1.82); 8.3156 (0.34); 7.2232 (0.42); 7.1971 (7.73); 7.1852 (4.3); 7.1756 (5.44); 7.1596 (0.49); 7.1527 (0.79);
6.5229 (4.58); 5.2252 (8.08); 3.9024 (2.68); 3.3252 (73.75); 2.6756 (0.62); 2.6712 (0.82); 2.6667 (0.6); 2.5414
(0.6); 2.5109 (57.73); 2.5067 (110.15); 2.5022 (140.04); 2.4977 (100.57); 2.4934 (49.11); 2.3334 (0.62); 2.3289
(0.83); 2.3244 (0.61); 2.2696 (16); 0.0079 (0.49); −0.0002 (13.21); −0.0084 (0.46)
Compound No. I-1-95, solvent: [DMSO], spectrometer: 399.95 MHz
11.1282 (2.97); 8.9443 (2.92); 8.9386 (2.87); 8.3634 (1.62); 8.3574 (1.58); 8.3426 (1.73); 8.3365 (1.65); 7.6565
(3.18); 7.6357 (3.01); 7.2195 (0.41); 7.1956 (9.77); 7.1815 (4.75); 7.1739 (5.42); 7.1584 (0.49); 7.1517 (0.75);
6.5231 (4.45); 5.2231 (7.89); 3.9022 (3.7); 3.3294 (84.6); 2.6716 (0.73); 2.6677 (0.56); 2.542 (0.87); 2.5071
(102.51); 2.5028 (127.82); 2.4985 (94.93); 2.3338 (0.61); 2.3296 (0.77); 2.2685 (16); −0.0002 (9.94); −0.0084
(0.46)
Compound No. I-1-62, solvent: [DMSO], spectrometer: 399.95 MHz
11.0412 (2.7); 9.106 (2.43); 8.7185 (1.75); 8.7095 (1.73); 8.3187 (1.57); 8.2986 (1.57); 7.5228 (1.39); 7.5109
(1.43); 7.5032 (1.39); 7.4912 (1.25); 7.2212 (0.43); 7.1971 (8.56); 7.1838 (4.47); 7.1753 (5.18); 7.1595 (0.47);
7.1523 (0.77); 6.5316 (4.21); 5.2246 (8.43); 3.9023 (1.31); 3.324 (63.08); 2.6752 (0.5); 2.6707 (0.61); 2.5059
(84.65); 2.5018 (105.45); 2.4975 (78.62); 2.328 (0.65); 2.2681 (16); −0.0002 (4.07)
Compound No. I-1-94, solvent: [DMSO], spectrometer: 399.95 MHz
10.8384 (1.81); 8.809 (1.8); 8.8034 (1.77); 8.2646 (1.21); 8.2584 (1.15); 8.2428 (1.25); 8.2366 (1.21); 7.4241
(0.49); 7.4088 (0.65); 7.4041 (0.91); 7.3889 (1.01); 7.3846 (0.73); 7.3691 (0.66); 7.1384 (0.45); 7.1319 (0.47);
7.1173 (0.76); 7.111 (0.78); 7.0949 (0.4); 7.09 (0.39); 6.9745 (1.18); 6.9528 (1.49); 6.946 (0.79); 6.9268 (0.68);
6.921 (0.76); 6.9008 (1.97); 6.8789 (1.92); 6.5246 (2.75); 5.2564 (4.72); 3.9141 (16); 3.9025 (1.74); 3.8934 (0.43);
3.3254 (60.55); 2.6756 (0.37); 2.6711 (0.49); 2.6665 (0.37); 2.5412 (0.39); 2.5244 (1.73); 2.5111 (32.77); 2.5067
(64.28); 2.5021 (82.83); 2.4975 (58.95); 2.4931 (27.93); 2.3334 (0.37); 2.3288 (0.48); 2.3244 (0.36); 2.2605
(9.56); −0.0002 (8.37)
Compound No. I-1-96, solvent: [DMSO], spectrometer: 399.95 MHz
11.2124 (3.08); 8.9024 (3.94); 8.8972 (3.92); 8.6353 (4.23); 8.63 (3.93); 8.3157 (0.36); 7.4255 (0.76); 7.41 (1.07);
7.4055 (1.45); 7.3902 (1.65); 7.386 (1.17); 7.3704 (1.09); 7.1419 (0.79); 7.1367 (0.8); 7.1206 (1.33); 7.1147
(1.29); 7.0997 (0.67); 7.0938 (0.65); 6.9811 (1.9); 6.9601 (2.66); 6.9296 (1.22); 6.5407 (4.72); 5.2676 (8.13);
3.9023 (3.41); 3.3244 (84.01); 2.6756 (0.68); 2.6711 (0.87); 2.6667 (0.63); 2.5413 (0.66); 2.511 (61.26); 2.5066
(117.71); 2.5021 (149.82); 2.4975 (106.16); 2.4931 (50.3); 2.3376 (0.36); 2.3333 (0.65); 2.3288 (0.89); 2.3242
(0.62); 2.2727 (16); 0.9866 (0.33); 0.0079 (0.6); −0.0002 (16.72); −0.0086 (0.49)
Compound No. I-1-108, solvent: [DMSO], spectrometer: 399.95 MHz
10.9469 (2.69); 9.0002 (2.68); 8.9951 (2.58); 8.3156 (0.38); 8.2218 (1.7); 8.216 (1.61); 8.2015 (1.75); 8.1957
(1.69); 7.4244 (0.82); 7.4089 (1.12); 7.4043 (1.53); 7.389 (1.68); 7.3847 (1.22); 7.3652 (2.81); 7.3449 (2.57);
7.1388 (0.77); 7.1332 (0.79); 7.1175 (1.28); 7.1113 (1.27); 7.0954 (0.64); 7.0906 (0.63); 6.9766 (1.93); 6.9553
(2.57); 6.9485 (1.29); 6.9295 (1.09); 6.9252 (1.19); 6.5361 (4.26); 5.2595 (8.02); 3.9023 (2.53); 3.3264 (71.21);
2.6804 (0.34); 2.6756 (0.58); 2.671 (0.78); 2.6664 (0.57); 2.5413 (0.99); 2.5249 (20.48); 2.52 (6.48); 2.511
(53.03); 2.5065 (103.25); 2.502 (132.69); 2.4974 (94.31); 2.4929 (44.68); 2.3378 (0.32); 2.3333 (0.59); 2.3287
(0.8); 2.3241 (0.58); 2.264 (16); 0.008 (0.56); −0.0002 (15.52); −0.0085 (0.47)
Compound No. I-1-107, solvent: [DMSO], spectrometer: 399.95 MHz
11.1546 (2.87); 9.0735 (2.92); 9.0692 (2.85); 8.87 (3.2); 8.8646 (3.21); 8.5668 (2.11); 8.5619 (3.36); 8.5568
(1.85); 8.3148 (0.52); 7.4262 (0.8); 7.4108 (1.07); 7.4061 (1.52); 7.3909 (1.67); 7.3865 (1.17); 7.3711 (1.1);
7.1418 (0.73); 7.1367 (0.76); 7.1205 (1.28); 7.1143 (1.29); 7.0983 (0.66); 7.0933 (0.63); 6.9823 (1.86); 6.9608
(2.36); 6.954 (1.3); 6.9294 (1.18); 6.5406 (4.45); 5.2661 (8.13); 3.9021 (3.63); 3.3285 (158.06); 2.6758 (0.69);
2.6714 (0.95); 2.667 (0.69); 2.6626 (0.35); 2.5416 (0.7); 2.5247 (2.92); 2.5114 (61.69); 2.5069 (121); 2.5024
(155.49); 2.4978 (110.3); 2.4933 (52.34); 2.3377 (0.33); 2.3337 (0.67); 2.3291 (0.9); 2.3245 (0.66); 2.3201 (0.33);
2.2703 (16); 0.008 (0.46); −0.0002 (14.88); −0.0085 (0.44)
Compound No. I-1-106, solvent: [DMSO], spectrometer: 399.95 MHz
11.1321 (2.84); 8.9478 (2.82); 8.9422 (2.8); 8.3674 (1.76); 8.3612 (1.71); 8.3465 (1.87); 8.3403 (1.83); 8.3155
(0.36); 7.6585 (3.2); 7.6382 (3); 7.4247 (0.76); 7.4095 (1.05); 7.4048 (1.47); 7.3895 (1.65); 7.3853 (1.18); 7.3698
(1.05); 7.1403 (0.75); 7.1345 (0.79); 7.119 (1.28); 7.1129 (1.3); 7.0971 (0.63); 7.0921 (0.64); 6.9778 (1.88);
6.9572 (2.74); 6.9314 (1.06); 6.9261 (1.19); 6.541 (4.34); 5.2646 (8.07); 3.9023 (3.18); 3.3253 (80.71); 2.6757
(0.6); 2.6713 (0.8); 2.6666 (0.6); 2.5414 (0.74); 2.5244 (2.66); 2.5111 (52.56); 2.5067 (103.02); 2.5022 (132.9);
2.4976 (95.61); 2.4932 (46.43); 2.3335 (0.57); 2.329 (0.77); 2.3245 (0.56); 2.2693 (16); 0.008 (0.42); −0.0002
(13.86); −0.0085 (0.48)
Compound No. I-1-60, solvent: [DMSO], spectrometer: 399.95 MHz
11.0462 (2.49); 9.1286 (0.95); 8.738 (0.86); 8.3248 (1.23); 8.316 (0.77); 8.3043 (1.28); 7.5326 (0.82); 7.5208
(0.95); 7.5136 (0.94); 7.5023 (0.76); 7.4266 (0.71); 7.4068 (1.45); 7.3912 (1.6); 7.3874 (1.2); 7.3715 (0.99);
7.1406 (0.75); 7.1351 (0.82); 7.1194 (1.33); 7.1137 (1.32); 7.0979 (0.69); 7.092 (0.68); 6.9809 (1.95); 6.96 (2.73);
6.9287 (1.29); 6.55 (4); 5.2655 (7.52); 3.9023 (1.9); 3.3284 (81.27); 2.6718 (0.69); 2.6675 (0.55); 2.5417 (0.63);
2.5071 (93.68); 2.5027 (118.77); 2.4983 (87.57); 2.3336 (0.54); 2.3295 (0.69); 2.2694 (16); 0.0079 (0.37); −0.0002
(10.28)
Compound No. I-1-3: mass (m/z): 217.1 (M + H)⁺; 1H NMR (DMSO-D₆) 2.25 (s, 3H), 3.66 (s, 3H), 6.41 (s, 1H),
7.47-7.50 (m, 1H), 8.27-8.30 (m, 1H), 8.69-8.70 (m, 1H), 9.10 (s, 1H), 10.69 (s, 1H).
Compound No. I-1-105, solvent: [DMSO], spectrometer: 399.95 MHz
10.8212 (1.84); 8.7951 (1.8); 8.7899 (1.74); 8.2521 (1.21); 8.2458 (1.15); 8.2303 (1.25); 8.224 (1.2); 7.3731
(0.33); 7.3687 (0.62); 7.3483 (0.74); 7.3437 (0.44); 7.3342 (0.4); 7.3298 (0.38); 7.2488 (0.77); 7.2465 (0.84);
7.2282 (0.7); 7.2251 (0.8); 7.223 (0.87); 7.2202 (0.91); 7.2019 (0.71); 7.1981 (1.12); 7.1949 (0.74); 7.1788 (1.52);
7.1762 (1.35); 7.1602 (0.84); 7.1574 (0.74); 7.0453 (0.58); 7.0413 (0.57); 7.0261 (0.99); 7.0221 (0.96); 7.007
(0.47); 7.0031 (0.43); 6.891 (1.94); 6.89 (1.92); 6.8693 (1.87); 6.8681 (1.85); 6.5124 (2.75); 5.2628 (4.43); 3.9083
(16); 3.9024 (2.33); 3.8933 (0.39); 3.3267 (51.97); 2.6756 (0.35); 2.6709 (0.47); 2.6665 (0.34); 2.5413 (0.44);
2.5242 (1.74); 2.5109 (31.79); 2.5065 (61.6); 2.502 (78.77); 2.4974 (55.83); 2.4929 (26.27); 2.3332 (0.38); 2.3287
(0.5); 2.3241 (0.4); 2.3018 (9.31); −0.0002 (8.08)
Compound No. I-1-103, solvent: [DMSO], spectrometer: 399.95 MHz
11.1922 (3.14); 8.8872 (4.07); 8.8819 (4.06); 8.6202 (4.34); 8.6149 (4.11); 8.3153 (0.4); 7.3911 (0.41); 7.3866
(0.44); 7.3771 (0.5); 7.3728 (1.07); 7.3525 (1.24); 7.3481 (0.7); 7.3385 (0.65); 7.3342 (0.62); 7.2468 (1.39);
7.2233 (1.45); 7.2205 (1.47); 7.1997 (2.25); 7.1808 (2.45); 7.1783 (2.22); 7.1622 (1.35); 7.1596 (1.25); 7.0717
(0.94); 7.0682 (0.94); 7.0527 (1.6); 7.0492 (1.55); 7.0337 (0.71); 7.0301 (0.68); 6.5269 (4.84); 5.272 (7.79);
3.9021 (2.28); 3.3246 (108.09); 3.1751 (0.8); 3.1621 (0.78); 2.6757 (0.64); 2.671 (0.87); 2.6665 (0.62); 2.5411
(0.6); 2.5244 (2.53); 2.511 (59.39); 2.5066 (116.61); 2.5021 (150.19); 2.4975 (107.66); 2.493 (51.85); 2.3333
(0.79); 2.3287 (1.19); 2.3159 (16); 0.008 (0.48); −0.0002 (15.2); −0.0085 (0.51)
Compound No. I-1-102, solvent: [DMSO], spectrometer: 399.95 MHz
10.9291 (2.71); 8.9851 (2.76); 8.9801 (2.64); 8.2075 (1.68); 8.2017 (1.57); 8.1872 (1.74); 8.1814 (1.65); 7.3876
(0.42); 7.3832 (0.49); 7.3689 (1.12); 7.3534 (3.55); 7.3442 (0.87); 7.3335 (3.06); 7.2487 (1.31); 7.2467 (1.35);
7.223 (1.49); 7.2206 (1.46); 7.1984 (1.84); 7.1789 (2.5); 7.1767 (2.21); 7.1604 (1.36); 7.1578 (1.2); 7.0497 (0.97);
7.0458 (0.95); 7.0305 (1.65); 7.0268 (1.56); 7.0113 (0.76); 7.0075 (0.69); 6.5239 (4.46); 5.2657 (7.8); 3.9021
(1.88); 3.3261 (86.91); 2.6755 (0.62); 2.671 (0.73); 2.6666 (0.52); 2.541 (0.65); 2.5182 (23.24); 2.5107 (52.33);
2.5064 (97.66); 2.5019 (122.92); 2.4974 (87.5); 2.4931 (42.05); 2.4654 (0.59); 2.3331 (0.64); 2.3286 (0.83);
2.3239 (0.73); 2.3056 (16); 0.0079 (0.42); −0.0002 (10.87); −0.0085 (0.34)
Compound No. I-1-101, solvent: [DMSO], spectrometer: 399.95 MHz
11.1372 (2.83); 9.0583 (2.95); 9.0541 (2.89); 8.861 (3.18); 8.8555 (3.16); 8.5523 (2.08); 8.5472 (3.33); 8.5422
(1.8); 7.3914 (0.42); 7.3873 (0.45); 7.3775 (0.5); 7.3727 (1.02); 7.3527 (1.21); 7.3481 (0.71); 7.3385 (0.63);
7.3343 (0.62); 7.2504 (1.3); 7.2482 (1.4); 7.2245 (1.43); 7.2219 (1.48); 7.2007 (2.16); 7.1815 (2.46); 7.179 (2.22);
7.163 (1.33); 7.1604 (1.21); 7.0682 (0.91); 7.0647 (0.91); 7.0492 (1.55); 7.0455 (1.52); 7.0298 (0.7); 6.5278
(4.59); 5.2714 (7.78); 3.9024 (2.89); 3.3248 (82.34); 3.1749 (0.6); 3.1619 (0.6); 2.6757 (0.62); 2.6711 (0.83);
2.6666 (0.62); 2.5412 (0.64); 2.5242 (2.67); 2.5109 (57.13); 2.5065 (112.07); 2.502 (144.38); 2.4974 (103.37);
2.493 (49.83); 2.3331 (0.74); 2.3287 (1.05); 2.3131 (16); 0.008 (0.44); −0.0002 (13.94); −0.0083 (0.45)
Compound No. I-1-93, solvent: [DMSO], spectrometer: 399.95 MHz
11.1147 (2.87); 8.932 (2.82); 8.9263 (2.82); 8.3529 (1.76); 8.3466 (1.71); 8.332 (1.84); 8.3257 (1.81); 8.3155
(0.42); 7.647 (3.19); 7.6261 (3.01); 7.3892 (0.41); 7.3847 (0.44); 7.3707 (1.03); 7.3506 (1.25); 7.346 (0.7); 7.3365
(0.64); 7.3323 (0.61); 7.2464 (1.4); 7.228 (1.1); 7.223 (1.42); 7.2203 (1.49); 7.1989 (2.16); 7.1798 (2.44); 7.1773
(2.25); 7.1612 (1.32); 7.1586 (1.24); 7.0601 (0.89); 7.0576 (0.91); 7.0418 (1.55); 7.0383 (1.52); 7.0225 (0.71);
7.019 (0.67); 6.5278 (4.49); 5.2698 (7.74); 3.9021 (2.23); 3.3243 (65.4); 3.1752 (0.53); 3.1621 (0.48); 2.6757
(0.61); 2.6711 (0.8); 2.6666 (0.59); 2.5412 (0.71); 2.5243 (2.55); 2.511 (51.35); 2.5066 (101.5); 2.5021 (131.31);
2.4975 (94.34); 2.4931 (45.52); 2.3332 (0.63); 2.3286 (0.9); 2.324 (0.89); 2.3119 (16); 0.008 (0.39); −0.0002
(13.77); −0.0085 (0.43)
Compound No. I-1-64, solvent: [DMSO], spectrometer: 399.95 MHz
11.0304 (2.52); 9.1001 (1.48); 8.7191 (1.12); 8.7096 (1.1); 8.3092 (1.35); 8.2893 (1.4); 7.517 (1.05); 7.5053
(1.11); 7.4975 (1.09); 7.4852 (0.96); 7.3899 (0.4); 7.3855 (0.42); 7.3711 (1.01); 7.3513 (1.24); 7.3465 (0.72);
7.3372 (0.64); 7.3328 (0.6); 7.2489 (1.34); 7.2253 (1.44); 7.2227 (1.48); 7.2009 (1.95); 7.1815 (2.41); 7.1792
(2.25); 7.1629 (1.32); 7.1606 (1.25); 7.0574 (0.9); 7.0538 (0.91); 7.038 (1.56); 7.0348 (1.52); 7.0194 (0.72);
6.5374 (4.24); 5.2717 (7.7); 3.9024 (1.78); 3.3264 (78.21); 2.6756 (0.5); 2.6713 (0.66); 2.6669 (0.48); 2.5416
(0.66); 2.5109 (45.32); 2.5066 (87.34); 2.5021 (111.86); 2.4976 (80.6); 2.4932 (39.32); 2.3334 (0.59); 2.3286
(0.8); 2.3241 (0.81); 2.3113 (16); 0.0078 (0.36); −0.0002 (9.58)
Compound No. I-1-104, solvent: [DMSO], spectrometer: 399.95 MHz
10.8316 (1.87); 8.8078 (1.93); 8.8021 (1.84); 8.2632 (1.22); 8.257 (1.14); 8.2414 (1.25); 8.2352 (1.21); 7.3631
(1.1); 7.3591 (0.52); 7.3455 (2.93); 7.3266 (2.26); 7.2903 (1.13); 7.2722 (1.38); 7.2659 (0.34); 7.2538 (0.43);
7.1473 (2.51); 7.1297 (2.11); 6.898 (2.04); 6.8762 (1.95); 6.5092 (2.89); 5.2287 (5.01); 3.9126 (16); 3.9022 (1.93);
3.8922 (0.35); 3.326 (66.93); 2.6756 (0.39); 2.6711 (0.51); 2.6665 (0.37); 2.541 (0.41); 2.5106 (35.11); 2.5064
(66.58); 2.5019 (84.65); 2.4974 (60.71); 2.4929 (29.41); 2.333 (0.38); 2.3287 (0.49); 2.324 (0.36); 2.2565 (10.02);
−0.0002 (7.86)
Compound No. I-1-100, solvent: [DMSO], spectrometer: 399.95 MHz
11.209 (3.14); 8.9021 (4.1); 8.8968 (4.12); 8.6325 (4.43); 8.6272 (4.16); 8.315 (0.43); 7.3642 (1.79); 7.3603 (0.8);
7.3468 (4.66); 7.3434 (2.53); 7.328 (3.63); 7.2935 (1.82); 7.2903 (1.16); 7.281 (0.7); 7.2753 (2.24); 7.2692 (0.53);
7.257 (0.64); 7.1528 (3.8); 7.1352 (3.24); 6.5257 (4.74); 5.2395 (8.29); 3.902 (4.15); 3.3267 (93.65); 2.6758
(0.66); 2.6712 (0.88); 2.6667 (0.63); 2.5415 (0.59); 2.5244 (2.81); 2.5112 (59.24); 2.5068 (114.82); 2.5022
(147.01); 2.4976 (104.25); 2.4931 (49.44); 2.3333 (0.69); 2.329 (0.89); 2.3241 (0.63); 2.2686 (16); 1.2981 (1.02);
1.2587 (1.45); 1.2344 (0.53); 0.008 (0.57); −0.0002 (14.86); −0.0085 (0.45)
Compound No. I-1-80, solvent: [DMSO], spectrometer: 399.95 MHz
10.9408 (2.6); 8.999 (2.69); 8.994 (2.63); 8.2205 (1.63); 8.2148 (1.58); 8.2002 (1.7); 8.1944 (1.67); 7.363 (4.27);
7.3455 (5.41); 7.3424 (5.05); 7.3269 (3.5); 7.2909 (1.74); 7.2727 (2.15); 7.2665 (0.5); 7.2543 (0.63); 7.1494
(3.92); 7.1318 (3.28); 6.521 (4.31); 5.2319 (8.25); 3.9021 (2.14); 3.3271 (78.93); 2.6799 (0.36); 2.6755 (0.56);
2.671 (0.73); 2.6665 (0.52); 2.5411 (0.81); 2.5236 (20.2); 2.5108 (49.41); 2.5064 (95); 2.5019 (121.11); 2.4974
(86.34); 2.4929 (41.45); 2.3332 (0.53); 2.3288 (0.7); 2.324 (0.51); 2.2597 (16); 1.2982 (1.04); 1.2587 (1.44); 0.008
(0.4); −0.0002 (11.26); −0.0085 (0.35)
Compound No. I-1-99, solvent: [DMSO], spectrometer: 399.95 MHz
11.1514 (2.76); 9.0736 (2.9); 9.0694 (2.81); 8.8678 (3.14); 8.8624 (3.09); 8.5647 (2.08); 8.5597 (3.28); 8.5547
(1.78); 8.3153 (0.4); 7.3652 (1.71); 7.3615 (0.77); 7.3477 (4.6); 7.3288 (3.62); 7.2939 (1.8); 7.2758 (2.24); 7.2698
(0.54); 7.2575 (0.71); 7.1542 (3.73); 7.1365 (3.22); 6.5259 (4.56); 5.2386 (8.28); 3.9022 (2.55); 3.3256 (104.59);
3.1752 (0.75); 3.1622 (0.75); 2.6802 (0.34); 2.6758 (0.67); 2.6711 (0.87); 2.6667 (0.65); 2.5414 (0.64); 2.5242
(2.8); 2.5109 (61.84); 2.5066 (119.12); 2.5021 (151.56); 2.4976 (107.87); 2.4932 (51.62); 2.3334 (0.65); 2.3288
(0.88); 2.3243 (0.65); 2.2662 (16); 1.2589 (0.49); 1.2346 (1.14); 0.0079 (0.43); −0.0002 (13.52); −0.0085 (0.41)
Compound No. I-1-98, solvent: [DMSO], spectrometer: 399.95 MHz
11.1275 (2.75); 8.9468 (2.8); 8.941 (2.82); 8.366 (1.62); 8.3598 (1.61); 8.345 (1.76); 8.3388 (1.72); 8.3152 (0.37);
7.6559 (3.14); 7.635 (2.99); 7.3638 (1.64); 7.3463 (4.52); 7.3274 (3.56); 7.2921 (1.78); 7.2791 (0.78); 7.2741
(2.21); 7.2557 (0.72); 7.1505 (3.77); 7.1328 (3.27); 6.5261 (4.44); 5.2368 (8.21); 4.02 (0.88); 3.902 (3.81); 3.5035
(0.43); 3.49 (0.53); 3.3265 (79.58); 2.6757 (0.5); 2.6713 (0.68); 2.6671 (0.48); 2.5415 (0.49); 2.5109 (49.41);
2.5067 (95.16); 2.5023 (121.54); 2.4978 (87.97); 2.4936 (43.42); 2.3333 (0.57); 2.3289 (0.75); 2.3245 (0.56);
2.2653 (16); 1.2587 (0.36); 0.0079 (0.37); −0.0002 (11.15); −0.0083 (0.33)
Compound No. I-1-46, solvent: [DMSO], spectrometer: 399.95 MHz
11.0396 (2.49); 9.1182 (1.29); 8.7288 (1.01); 8.3221 (1.31); 8.3155 (0.98); 8.3021 (1.36); 7.5267 (1.01); 7.5149
(1.07); 7.5072 (1.04); 7.4952 (0.91); 7.3656 (1.72); 7.3481 (4.56); 7.3292 (3.54); 7.293 (1.78); 7.2803 (0.78);
7.2748 (2.2); 7.2564 (0.7); 7.1534 (3.78); 7.1357 (3.23); 6.5348 (4.08); 5.2379 (7.99); 4.0197 (0.51); 3.902 (1.08);
3.4895 (0.34); 3.327 (117.37); 2.6803 (0.36); 2.6758 (0.62); 2.6713 (0.81); 2.6667 (0.6); 2.5415 (0.84); 2.5111
(56.65); 2.5068 (107.56); 2.5023 (136.51); 2.4977 (98.29); 2.4934 (47.9); 2.3334 (0.6); 2.329 (0.77); 2.3245
(0.57); 2.2653 (16); 1.2346 (1.2); −0.0002 (5.56)
Compound No. I-1-140 (Synthesis Example 9), solvent: [DMSO], spectrometer: 399.95 MHz
11.267 (4.96); 9.1112 (4.57); 9.1071 (4.48); 8.8097 (13.79); 8.7974 (13.97); 8.7378 (3.51); 8.7338 (3.65); 8.7258
(3.74); 8.7217 (3.58); 8.3298 (1.91); 8.3251 (2.64); 8.3199 (1.83); 8.3155 (0.52); 8.3098 (2.08); 8.3047 (2.78);
8.2999 (1.86); 7.5381 (2.49); 7.5365 (2.43); 7.5261 (2.45); 7.5244 (2.44); 7.5182 (2.44); 7.5166 (2.33); 7.5062
(2.35); 7.5045 (2.26); 7.478 (3.09); 7.4657 (5.94); 7.4535 (2.94); 6.8529 (10.98); 5.5438 (16); 3.322 (29.51);
3.1756 (0.32); 3.1625 (0.33); 2.671 (0.38); 2.5244 (0.9); 2.5109 (23.53); 2.5065 (46.44); 2.5019 (60.99); 2.4974
(44.43); 2.4929 (21.07); 2.3287 (0.41); 0.0079 (1.45); −0.0002 (38.86); −0.0085 (1.3)
Compound No. I-1-134 (Synthesis Example 2), solvent: [DMSO], spectrometer: 600.13 MHz
11.4298 (2.04); 9.0149 (2.36); 8.7884 (2.36); 8.7838 (2.4); 8.2606 (0.71); 8.2575 (0.9); 8.2562 (0.89); 8.2532
(0.71); 8.2446 (0.74); 8.2416 (0.93); 8.2403 (0.87); 8.2372 (0.69); 7.1952 (4.33); 4.067 (16); 3.8634 (15.57);
3.3277 (6.53); 2.5124 (2.52); 2.5095 (5.35); 2.5065 (7.33); 2.5036 (5.31); 2.5007 (2.49)
Compound No. I-1-138, solvent: [DMSO], spectrometer: 399.95 MHz
11.3355 (1.63); 9.129 (1.76); 9.125 (1.76); 8.7542 (1.4); 8.7501 (1.49); 8.7422 (1.49); 8.7381 (1.46); 8.3454
(0.77); 8.3408 (1.04); 8.3355 (0.76); 8.3255 (0.83); 8.3201 (1.08); 8.3156 (0.82); 7.5578 (0.98); 7.556 (0.98);
7.5457 (0.96); 7.5439 (0.97); 7.5379 (0.96); 7.5361 (0.95); 7.5259 (0.92); 7.524 (0.91); 7.2032 (4.63); 4.1001
(0.45); 4.0869 (0.49); 4.0639 (16); 3.8616 (15.2); 3.3238 (15.96); 3.1756 (2.79); 3.1625 (2.7); 2.5249 (0.58); 2.52
(0.91); 2.5115 (11.33); 2.507 (22.88); 2.5024 (30.11); 2.4978 (21.37); 2.4933 (10.01); 0.008 (0.98); −0.0002
(27.98); −0.0086 (0.94)
Compound No. I-1-135, solvent: [DMSO], spectrometer: 399.95 MHz
11.2558 (2.83); 9.0026 (2.12); 8.999 (3.62); 8.7683 (3.79); 8.7614 (3.84); 8.2628 (1.09); 8.2581 (1.35); 8.2562
(1.32); 8.2516 (1.07); 8.2386 (1.12); 8.2339 (1.37); 8.2274 (1.03); 6.602 (4.59); 5.0416 (1.13); 5.0188 (3.64);
4.9959 (3.81); 4.9729 (1.3); 3.3263 (17.64); 3.1767 (0.4); 3.1635 (0.4); 2.5255 (0.51); 2.5122 (9.6); 2.5078 (19.1);
2.5033 (24.92); 2.4987 (17.71); 2.4943 (8.46); 2.3253 (16); −0.0002 (4.52)
Compound No. I-1-133, solvent: [DMSO], spectrometer: 600.13 MHz
11.1513 (2.75); 9.1183 (2.84); 9.1154 (2.88); 8.7333 (2.19); 8.7306 (2.27); 8.7253 (2.3); 8.7227 (2.25); 8.3341
(1.09); 8.3311 (1.63); 8.3278 (1.1); 8.3209 (1.17); 8.3177 (1.69); 8.3145 (1.1); 7.5307 (1.62); 7.5297 (1.54);
7.5227 (1.62); 7.5217 (1.55); 7.5175 (1.64); 7.5165 (1.53); 7.5095 (1.57); 7.5084 (1.47); 6.6052 (4.19); 5.0229
(0.99); 5.0077 (3.17); 4.9925 (3.36); 4.9772 (1.17); 3.3306 (14.88); 2.5078 (8.17); 2.5049 (11.1); 2.5019 (8.09);
2.3249 (16)
Compound No. I-1-132, solvent: [DMSO], spectrometer: 600.13 MHz
11.6586 (1.39); 9.0707 (0.9); 9.0682 (1.5); 9.066 (0.86); 8.7889 (1.48); 8.7843 (1.49); 8.321 (0.46); 8.318 (0.56);
8.3165 (0.54); 8.3135 (0.44); 8.3049 (0.48); 8.3019 (0.58); 8.3004 (0.53); 8.2974 (0.43); 7.2082 (2.24); 6.8361
(1.7); 3.328 (5.08); 2.665 (6.28); 2.513 (2.02); 2.51 (4.26); 2.507 (5.74); 2.504 (4.15); 2.5011 (1.93); 2.4699 (16)
Compound No. I-1-12: mass (m/z): 259.1 (M + H)⁺; 1H NMR (DMSO-D₆) 0.87-0.89 (d, 6H), 2.10-2.16 (m, 1H),
2.26 (s, 3H), 3.75-3.76 (d, 2H), 6.42 (s, 1H), 7.46-7.49 (m, 1H), 8.28-8.31 (m, 1H), 8.68-8.70 (m, 1H),
9.10 (s, 1H), 10.74 (s, 1H).
Compound No. I-1-139, solvent: [DMSO], spectrometer: 399.95 MHz
11.5553 (1.44); 9.1799 (1.3); 9.1759 (1.29); 9.1744 (1.2); 8.753 (0.97); 8.749 (1.02); 8.741 (1.03); 8.737 (1);
8.4029 (0.55); 8.3983 (0.75); 8.393 (0.54); 8.3829 (0.6); 8.3776 (0.79); 8.373 (0.55); 7.5561 (0.68); 7.5544 (0.68);
7.544 (0.67); 7.5424 (0.68); 7.5362 (0.67); 7.5345 (0.66); 7.5242 (0.64); 7.5224 (0.64); 7.2068 (2.35); 6.8408
(1.87); 6.8394 (1.85); 3.3232 (1.88); 2.6635 (6.38); 2.6624 (6.3); 2.5125 (4.34); 2.5081 (8.58); 2.5035 (11.36);
2.499 (8.39); 2.4945 (4.03); 2.4697 (16); −0.0002 (7.34)
Compound No. I-1-137, solvent: [DMSO], spectrometer: 399.95 MHz
10.5164 (3.25); 9.1054 (2.76); 8.8206 (12.35); 8.8083 (12.48); 8.7684 (2.72); 8.7651 (2.84); 8.7565 (2.84); 8.7532
(2.68); 8.303 (1.48); 8.285 (1.52); 8.1711 (10.82); 7.567 (2.02); 7.5549 (2.08); 7.5471 (2.05); 7.535 (1.87); 7.4875
(3.02); 7.4752 (5.76); 7.4629 (2.9); 5.5272 (16); 4.038 (0.81); 4.0203 (0.8); 3.3238 (34.5); 2.6762 (0.4); 2.671
(0.52); 2.5409 (0.44); 2.5064 (67.46); 2.502 (86.29); 2.4977 (62.17); 2.3335 (0.41); 2.3288 (0.55); 2.3245 (0.39);
1.989 (3.48); 1.2362 (0.57); 1.1927 (0.9); 1.1749 (1.79); 1.1571 (0.89); 0.0082 (0.32); −0.0002 (8.46)
Compound No. I-1-136 (Synthesis Example 11), solvent: [DMSO], spectrometer: 399.95 MHz
10.3936 (2.65); 9.1082 (2.55); 8.8035 (11.47); 8.7912 (11.64); 8.7553 (0.37); 8.7425 (2.15); 8.7386 (2.28); 8.7303
(2.06); 8.7269 (1.99); 8.3158 (0.39); 8.3039 (1.4); 8.2846 (1.43); 7.6604 (5.74); 7.5436 (1.41); 7.5313 (1.52);
7.5239 (1.48); 7.5116 (1.33); 7.4638 (2.83); 7.4515 (5.35); 7.4392 (2.66); 5.4369 (12.53); 5.147 (0.35); 3.3264
(39.31); 3.1758 (1.05); 3.1627 (1.04); 2.5243 (0.91); 2.511 (18.16); 2.5066 (36.92); 2.502 (48.86); 2.4975 (34.95);
2.493 (16.73); 1.9163 (16); 1.8877 (0.56); 1.8169 (0.52); −0.0002 (8.8
Compound No. I-1-125, solvent: [DMSO], spectrometer: 601.6 MHz
10.4392 (0.79); 9.1036 (0.65); 8.8124 (5.6); 8.8043 (5.77); 8.7631 (0.8); 8.7611 (0.84); 8.7555 (0.79); 8.7534
(0.75); 8.2959 (0.37); 8.2843 (0.39); 7.5591 (0.53); 7.551 (0.58); 7.5461 (0.58); 7.538 (0.5); 7.4756 (1.2); 7.4674
(2.32); 7.4592 (1.16); 5.7562 (16); 5.5703 (5.19); 4.289 (0.47); 3.4391 (0.32); 3.3247 (11.38); 2.5088 (3.98);
2.5058 (8.7); 2.5027 (12.01); 2.4996 (8.77); 2.4966 (4.01); 2.309 (8.98); 2.161 (0.46); 1.9897 (1.34); 1.8273
(2.25); 1.187 (0.37); 1.1751 (0.72); 1.1633 (0.37); −0.0002 (2.26)
Compound No. I-1-20, solvent: [DMSO], spectrometer: 399.95 MHz
10.9775 (2.88); 9.0736 (2.7); 9.0686 (2.8); 8.7994 (1.99); 8.7953 (2.12); 8.7871 (2.09); 8.783 (2.06); 8.7027
(1.96); 8.6989 (2.08); 8.6908 (2.06); 8.6869 (2); 8.3752 (2.01); 8.3711 (2.01); 8.3554 (2.15); 8.3513 (2.07); 8.294
(1.02); 8.2898 (1.54); 8.2742 (1.11); 8.2698 (1.61); 7.5839 (1.94); 7.5716 (1.91); 7.5642 (1.86); 7.5519 (1.78);
7.5007 (1.56); 7.4886 (1.54); 7.4808 (1.53); 7.4687 (1.44); 6.5362 (4.37); 5.5075 (9.9); 3.3237 (14.04); 2.5063
(37.65); 2.5019 (48); 2.4975 (34.73); 2.4027 (16); 1.9889 (0.36); −0.0002 (7.41)
Compound No. I-1-37, solvent: [DMSO], spectrometer: 399.95 MHz
10.9663 (2.74); 9.0819 (2.71); 9.0767 (2.66); 8.7049 (1.92); 8.7011 (2.1); 8.693 (2.02); 8.6891 (2.03); 8.4789
(2.05); 8.4757 (2.22); 8.4673 (2.13); 8.464 (2.19); 8.3012 (0.99); 8.2966 (1.53); 8.2919 (1.03); 8.2813 (1.08);
8.2765 (1.62); 8.272 (1.03); 7.9762 (2.03); 7.9729 (2.12); 7.9559 (2.22); 7.9526 (2.22); 7.5036 (1.53); 7.4916
(1.54); 7.4837 (1.49); 7.4716 (1.42); 7.4106 (1.92); 7.3989 (1.91); 7.3903 (1.81); 7.3787 (1.75); 6.519 (4.28);
5.4463 (10.01); 3.3236 (4.61); 2.5062 (25.75); 2.5019 (33.44); 2.4975 (24.42); 2.3391 (16); −0.0002 (6.15)
Compound No. I-1-123, solvent: [CD3CN], spectrometer: 601.6 MHz
9.0535 (0.94); 9.0296 (0.49); 9.0282 (0.56); 9.024 (1.44); 9.0198 (1.35); 8.7487 (0.48); 8.7451 (1.07); 8.7422
(1.07); 8.7371 (1.15); 8.7343 (1.11); 8.7196 (0.94); 8.7128 (6.78); 8.707 (6.36); 8.7047 (6.51); 8.6988 (5.84);
8.6383 (1.12); 8.6302 (1.13); 8.5925 (0.73); 8.5844 (0.83); 8.5741 (0.39); 8.1994 (0.5); 8.1884 (0.73); 8.1854
(0.8); 8.1819 (1.06); 8.1787 (0.99); 8.1753 (0.9); 8.1722 (0.52); 8.1717 (0.51); 8.1687 (0.86); 8.1655 (0.87);
8.1622 (0.6); 7.4821 (0.71); 7.4741 (0.81); 7.4699 (0.91); 7.4688 (0.93); 7.4609 (1.29); 7.457 (0.58); 7.4553
(0.63); 7.4533 (0.68); 7.4473 (0.9); 7.4458 (0.73); 7.4392 (0.7); 7.4378 (0.58); 7.434 (0.37); 7.4326 (0.37); 7.3182
(1.52); 7.3155 (1.5); 7.3101 (3.13); 7.3073 (2.74); 7.3019 (1.56); 7.2991 (1.45); 7.1971 (0.48); 5.4461 (7.81);
5.4001 (7.55); 5.348 (6.65); 4.8207 (1.66); 4.7621 (0.89); 4.5171 (0.34); 4.5081 (0.63); 4.4988 (0.37); 4.4202
(0.64); 4.4114 (1.02); 4.4022 (0.73); 3.8819 (0.52); 3.8116 (0.5); 3.8026 (0.84); 3.7937 (0.48); 2.1966 (12.66);
2.1741 (2.37); 2.1273 (10.18); 2.0495 (0.49); 2.0048 (4.32); 1.9833 (0.44); 1.9753 (0.59); 1.9711 (0.39); 1.9632
(3.05); 1.9573 (0.85); 1.9551 (4.38); 1.9511 (4.63); 1.9471 (22.58); 1.9431 (39.14); 1.939 (55.5); 1.9348 (35.72);
1.9307 (18.86); 1.9261 (0.51); 1.8998 (16); 1.868 (0.48); 1.8242 (0.35); 1.8098 (0.56); 0.0053 (1.44); −0.0002
(51.4); −0.0057 (1.35)
Compound No. I-1-36, solvent: [DMSO], spectrometer: 399.95 MHz
10.9929 (1.81); 9.0976 (1.9); 8.7122 (1.52); 8.3102 (1.26); 8.2921 (0.97); 7.5152 (1.24); 7.5036 (1.22); 7.4968
(1.04); 7.484 (0.68); 7.1712 (2.24); 6.5152 (2.02); 5.2989 (4.26); 3.3548 (0.67); 3.3249 (3.21); 2.7881 (1.14);
2.7481 (1.07); 2.7436 (1.07); 2.6584 (1.76); 2.5045 (19.91); 2.3751 (16); 2.3392 (6.89); 0.0066 (3.35); −0.0002
(4.77)
Compound No. I-1-53, solvent: [DMSO], spectrometer: 399.95 MHz
10.9859 (1.21); 9.0997 (1.07); 9.0947 (1.15); 8.7157 (0.7); 8.712 (0.84); 8.7038 (0.87); 8.7 (0.93); 8.3168 (0.39);
8.3121 (0.62); 8.3077 (0.47); 8.297 (0.46); 8.292 (0.67); 8.2877 (0.49); 7.5174 (0.6); 7.5054 (0.66); 7.4975 (0.64);
7.4855 (0.62); 6.5342 (1.69); 6.1575 (2.69); 6.1455 (0.34); 5.2292 (3.72); 3.8168 (16); 3.8048 (1.99); 3.3247
(4.68); 3.3127 (0.72); 2.5066 (9.78); 2.5023 (13.64); 2.4981 (11.21); 2.3738 (6.2); 2.363 (0.91); −0.0002 (7.35); −0.0084
(0.68); −0.0123 (0.91)
Compound No. I-1-124, solvent: [DMSO], spectrometer: 399.95 MHz
10.986 (2.92); 9.108 (2.79); 9.1037 (2.77); 8.7265 (2.14); 8.7225 (2.32); 8.7145 (2.28); 8.7105 (2.28); 8.3246
(1.07); 8.3197 (1.59); 8.315 (1.16); 8.3047 (1.18); 8.2996 (1.68); 8.2949 (1.11); 7.9743 (1.11); 7.9629 (1.15);
7.5314 (1.53); 7.5298 (1.56); 7.5193 (1.54); 7.5178 (1.59); 7.5115 (1.52); 7.5099 (1.53); 7.4995 (1.49); 7.4978
(1.52); 6.4799 (4.32); 4.6267 (9.57); 3.3247 (17.09); 2.6224 (9.5); 2.6109 (9.47); 2.5243 (0.75); 2.5108 (14.68);
2.5065 (29.66); 2.502 (39.13); 2.4974 (28.3); 2.493 (13.72); 2.2374 (16); 1.9888 (1.07); 1.1748 (0.56); −0.0002
(1.32)
Compound No. I-1-31, solvent: [DMSO], spectrometer: 399.95 MHz
10.9873 (1.26); 9.0963 (2.09); 9.0914 (2.14); 8.7135 (1.57); 8.7096 (1.72); 8.7016 (1.69); 8.6975 (1.69); 8.6073
(3.09); 8.5945 (3.16); 8.3136 (0.79); 8.309 (1.22); 8.304 (0.83); 8.2937 (0.87); 8.2889 (1.3); 8.2841 (0.85); 8.2547
(1.12); 7.5156 (1.18); 7.5035 (1.18); 7.4958 (1.17); 7.4837 (1.1); 7.3123 (2.36); 7.2995 (2.3); 6.5204 (3.37);
5.3607 (7.7); 2.9497 (0.83); 2.5246 (0.42); 2.5113 (8.85); 2.507 (17.66); 2.5025 (23.76); 2.4981 (17.08); 2.4938
(8.47); 2.4454 (16); 2.3249 (12.62); −0.0002 (0.67)
Compound No. I-1-57, solvent: [DMSO], spectrometer: 399.95 MHz
10.8674 (2.49); 9.0649 (1.92); 8.8583 (2.09); 8.8517 (2.11); 8.3162 (0.35); 8.2619 (0.96); 8.2394 (0.94); 3.969
(14.69); 3.3979 (0.46); 3.3248 (923.39); 2.9946 (0.82); 2.6747 (3.1); 2.6704 (4.08); 2.666 (3.03); 2.5845 (16);
2.5408 (23.83); 2.5058 (485.46); 2.5013 (615.9); 2.4969 (451.2); 2.3972 (12.1); 2.3326 (2.92); 2.3281 (3.88);
2.3237 (2.83); 2.2875 (0.37); 2.2392 (14.31); 2.0744 (0.49); 1.2583 (0.41); 1.2353 (2.82); −0.0002 (64.79); −0.0083
(2.95)
Compound No. I-1-119, solvent: [DMSO], spectrometer: 399.95 MHz
11.0434 (1.56); 9.2061 (1.44); 9.2021 (1.43); 8.7947 (1.23); 8.7907 (1.31); 8.7827 (1.3); 8.7786 (1.26); 8.4077
(0.59); 8.4031 (0.87); 8.3984 (0.59); 8.3877 (0.64); 8.3831 (0.91); 7.7767 (1.59); 7.7562 (1.7); 7.6322 (1.68);
7.6108 (2.12); 7.6043 (1.03); 7.5922 (0.94); 7.5908 (0.94); 7.5843 (0.93); 7.5723 (0.89); 7.4413 (0.92); 7.4388
(0.94); 7.4243 (1.1); 7.4214 (1.28); 7.4175 (0.82); 7.4029 (0.86); 7.4004 (0.84); 7.1357 (1.04); 7.1341 (1.06);
7.1161 (1.39); 7.0981 (0.9); 7.0965 (0.86); 4.0194 (16); 3.3245 (87.06); 2.6751 (0.43); 2.6704 (0.59); 2.666 (0.43);
2.5408 (15.15); 2.5238 (1.58); 2.5104 (34.23); 2.506 (69.29); 2.5015 (91.48); 2.4969 (65.92); 2.4924 (31.4);
2.3328 (0.43); 2.3283 (0.58); 2.3236 (0.41); 1.235 (0.41); 0.008 (0.55); −0.0002 (17.1); −0.0085 (0.52)
Compound No. I-1-18 (Synthesis Example 3), solvent: [DMSO], spectrometer: 399.95 MHz
10.8077 (0.94); 9.0862 (0.92); 9.0819 (0.9); 9.0805 (0.89); 8.8312 (0.66); 8.8273 (0.71); 8.8192 (0.71); 8.8152
(0.7); 8.279 (0.37); 8.2737 (0.51); 8.2691 (0.38); 8.2591 (0.41); 8.2536 (0.56); 8.2491 (0.4); 7.6518 (0.47); 7.6502
(0.49); 7.6397 (0.47); 7.6382 (0.49); 7.6319 (0.46); 7.6303 (0.47); 7.6198 (0.44); 7.6182 (0.45); 6.6089 (2.25);
3.3238 (4.68); 2.5114 (4); 2.507 (7.91); 2.5024 (10.35); 2.4979 (7.53); 2.4934 (3.68); 2.214 (6.07); 1.5586 (16); −0.0002
(2.41)
Compound No. I-1-27: mass (m/z): 314.1 (M + H)⁺; 1H NMR (DMSO-D₆) 5.32 (s, 2H), 6.66-6.67 (d, 1H),
7.46-7.50 (m, 2H), 7.69-7.72 (m, 1H), 7.81-7.82 (d, 1H), 8.26-8.30 (m, 1H), 8.35 (s, 1H), 8.69-8.70 (m, 1H),
9.09 (s, 1H), 10.88 (s, 1H).
Compound No. I-1-17 (Synthesis Example 4), solvent: [DMSO], spectrometer: 399.95 MHz
11.5244 (2.09); 9.3343 (2.9); 9.3301 (2.79); 8.9696 (2.07); 8.9662 (2.16); 8.9562 (2.16); 8.9528 (2.07); 8.8224
(1.44); 8.8021 (1.46); 7.9797 (1.38); 7.9661 (1.42); 7.9596 (1.36); 7.9461 (1.24); 6.4467 (4.23); 2.5271 (0.9);
2.5138 (14.54); 2.5095 (28.07); 2.5049 (36.1); 2.5004 (25.87); 2.4959 (12.29); 2.2701 (16); −0.0002 (2.81)
Compound No. I-2-1, solvent: [DMSO], spectrometer: 399.95 MHz
13.2623 (0.61); 13.2552 (0.59); 9.1661 (1.61); 9.1319 (4.59); 8.776 (6.31); 8.7671 (6.28); 8.7644 (6.06); 8.5652
(0.57); 8.5502 (0.6); 8.342 (2.69); 8.3292 (2.73); 7.5779 (4.86); 7.5658 (5.17); 7.5582 (5.05); 7.5461 (4.54);
7.2679 (0.8); 7.253 (0.79); 3.3295 (4.56); 3.1701 (0.55); 2.7642 (5.84); 2.6769 (0.54); 2.6723 (0.74); 2.6678
(0.55); 2.5424 (0.54); 2.5255 (2.65); 2.5121 (42.88); 2.5078 (84.73); 2.5033 (110.99); 2.4987 (80.56); 2.4943
(39.26); 2.35 (0.72); 2.3341 (1.33); 2.3299 (1.64); 2.3253 (1.61); 2.3207 (1.52); 2.2744 (8.52); 2.0893 (16); 1.9895
(0.51); 0.0077 (1.85); −0.0002 (49.21); −0.0084 (1.81)
Compound No. I-1-35 (Synthesis Example 10): 1H NMR (DMSO-D₆) 1.17 (t, 3H), 2.62 (q, 2H), 5.23 (s, 2H),
6.54 (s, 1H), 7.13-7.35 (m. 5H). 7.46-7.50 (m, 1H), 8.29-8.32 (m, 1H), 8.69-8.70 (m, 1H), 9.11 (s, 1H), 10.81 (s,
1H).
Compound No. I-1-52, solvent: [DMSO], spectrometer: 399.95 MHz
11.4819 (10.59); 9.179 (9.97); 9.1774 (10.3); 9.1734 (10.58); 9.1717 (9.58); 8.7727 (8.4); 8.7686 (8.98); 8.7607
(8.95); 8.7566 (8.75); 8.6099 (16); 8.6033 (15.93); 8.4887 (5.51); 8.4868 (6.16); 8.4842 (6.62); 8.4822 (5.71);
8.4766 (5.86); 8.4746 (6.49); 8.4721 (6.39); 8.4701 (5.52); 8.3946 (4.86); 8.3902 (6.53); 8.3846 (4.78); 8.3747
(5.24); 8.3702 (6.63); 8.3691 (6.5); 8.3647 (4.92); 8.3177 (0.32); 8.2591 (0.43); 8.2525 (0.4); 8.0305 (4.45);
8.0259 (4.41); 8.012 (5.85); 8.0098 (6.62); 8.0074 (6.02); 8.0053 (6.24); 7.9914 (5.9); 7.9868 (5.72); 7.8477
(0.38); 7.8368 (11.7); 7.8161 (9.56); 7.8141 (5.36); 7.5979 (0.34); 7.5785 (6.37); 7.5765 (6.2); 7.5664 (6.08);
7.5645 (5.92); 7.5586 (6.04); 7.5566 (5.78); 7.5465 (5.87); 7.5445 (5.61); 7.362 (5.87); 7.3596 (5.92); 7.3498
(5.61); 7.3474 (6.05); 7.3436 (6.08); 7.3412 (5.4); 7.3314 (5.67); 7.329 (5.34); 7.0181 (14.8); 7.0114 (14.62); 5.8
(0.48); 5.7934 (0.46); 5.2497 (0.43); 3.3283 (75.47); 2.6778 (0.52); 2.6733 (0.72); 2.6687 (0.51); 2.5266 (2.17);
2.5218 (3.38); 2.5133 (39.29); 2.5088 (78.93); 2.5042 (104.18); 2.4996 (75.12); 2.4951 (35.55); 2.3356 (0.49);
2.3309 (0.69); 2.3264 (0.48); 1.3368 (0.37); 1.25 (0.5); 0.0079 (2.47); −0.0002 (70.38); −0.0086 (2.23)
Compound No. I-1-30, solvent: [DMSO], spectrometer: 399.95 MHz
11.5021 (11.76); 9.1701 (11.28); 9.166 (11.23); 8.7641 (8.87); 8.76 (9.52); 8.752 (9.43); 8.7479 (9.28); 8.4344
(11.84); 8.4333 (12.02); 8.4277 (11.98); 8.4267 (11.77); 8.3935 (5.74); 8.3893 (12.03); 8.3869 (13.88); 8.3837
(10.85); 8.378 (6.61); 8.3745 (13.07); 8.3687 (8.01); 8.3637 (5.35); 8.2362 (0.36); 8.233 (0.63); 8.2296 (0.36);
8.2246 (0.37); 8.2214 (0.62); 8.2183 (0.35); 8.1404 (0.85); 8.1338 (0.81); 8.04 (5.02); 8.0366 (5.07); 8.0194
(5.65); 8.016 (5.53); 8.0112 (5.26); 8.0077 (5.22); 7.9905 (5.5); 7.9871 (5.37); 7.8674 (0.34); 7.8638 (0.33);
7.8469 (0.35); 7.8434 (0.36); 7.8371 (0.35); 7.8337 (0.32); 7.8167 (0.34); 7.8132 (0.34); 7.5678 (6.46); 7.566
(6.51); 7.5557 (6.38); 7.554 (6.42); 7.5478 (6.29); 7.5461 (6.27); 7.5358 (6.19); 7.534 (6.32); 7.5294 (5.73);
7.5208 (6.09); 7.5178 (5.7); 7.509 (9.52); 7.5002 (5.04); 7.4972 (5.13); 7.4887 (4.55); 7.3063 (0.44); 7.2983
(0.44); 7.2948 (0.45); 7.2863 (0.74); 7.2779 (0.46); 7.2745 (0.4); 7.2662 (0.43); 7.0478 (16); 7.041 (15.84); 5.833
(0.96); 5.8263 (0.96); 5.2229 (1.09); 3.3267 (75.69); 3.1771 (0.55); 3.164 (0.55); 2.6772 (0.62); 2.6727 (0.87);
2.6682 (0.64); 2.6638 (0.32); 2.526 (2.75); 2.5211 (4.31); 2.5127 (47.37); 2.5082 (94.59); 2.5036 (124.74); 2.4991
(90.44); 2.4945 (43.34); 2.3349 (0.58); 2.3304 (0.81); 2.3259 (0.57); 1.259 (0.37); 1.25 (0.41); 1.2322 (0.32);
0.1459 (0.37); 0.0079 (2.96); −0.0002 (80.32); −0.0084 (2.67); −0.1497 (0.35)
Compound No. I-1-117, solvent: [DMSO], spectrometer: 399.95 MHz
11.475 (11.08); 9.1604 (9.76); 9.1572 (9.64); 8.7644 (6.68); 8.7553 (6.64); 8.3881 (12.97); 8.3819 (16); 8.3575
(6.41); 8.036 (7.02); 8.0168 (7.52); 7.8979 (2.86); 7.8953 (2.87); 7.8773 (7.15); 7.8591 (5.76); 7.8565 (5.62);
7.8247 (10.78); 7.8052 (5.6); 7.6024 (4.83); 7.5839 (8.48); 7.5725 (6.57); 7.5621 (7.36); 7.553 (5.76); 7.5408
(4.91); 7.4142 (0.32); 7.3931 (0.41); 7.3654 (0.44); 7.0541 (11.38); 7.0479 (11.15); 5.7567 (1.47); 3.7424 (0.36);
3.3278 (68.26); 2.6716 (1.06); 2.5027 (148.84); 2.3294 (1.05); 1.9911 (0.59); 1.4917 (0.4); 1.3367 (0.6); 1.2989
(4.37); 1.259 (5.39); 1.2335 (0.82); −0.0002 (51.23)
Compound No. I-1-39, solvent: [DMSO], spectrometer: 399.95 MHz
11.3674 (1.9); 9.2176 (1.89); 9.2132 (1.91); 8.8012 (1.46); 8.7972 (1.54); 8.7892 (1.54); 8.7851 (1.49); 8.5859
(1.66); 8.5819 (1.81); 8.5748 (1.77); 8.5708 (1.74); 8.4233 (0.83); 8.4186 (1.2); 8.4134 (0.91); 8.4076 (1.81);
8.4036 (2.45); 8.3987 (1.35); 8.3935 (0.93); 8.3872 (1.8); 8.3832 (1.64); 7.6086 (1.1); 7.607 (1.07); 7.5965 (1.1);
7.5949 (1.07); 7.5887 (1.09); 7.5871 (1.03); 7.5767 (1.03); 7.575 (0.98); 7.2298 (1.73); 7.2187 (1.72); 7.2093
(1.71); 7.1982 (1.67); 4.0402 (16); 3.75 (0.59); 3.3283 (7.67); 3.1777 (0.44); 3.1646 (0.43); 2.5261 (0.41); 2.5128
(6.6); 2.5085 (12.79); 2.5039 (16.63); 2.4994 (12.16); 2.495 (5.98); −0.0002 (0.93)
Compound No. I-1-63, solvent: [DMSO], spectrometer: 399.95 MHz
11.511 (9.56); 9.1672 (8.52); 9.1629 (8.54); 8.9527 (7.08); 8.9507 (8.31); 8.9478 (8.14); 8.7696 (6.84); 8.7655
(7.27); 8.7576 (7.34); 8.7535 (7.46); 8.7447 (8.67); 8.7403 (8.38); 8.4333 (15.96); 8.4264 (16); 8.3901 (3.79);
8.3854 (5.26); 8.3802 (3.7); 8.3702 (4.09); 8.3653 (5.49); 8.3603 (3.75); 8.3165 (0.35); 7.5733 (4.92); 7.5717
(4.78); 7.5613 (4.81); 7.5596 (4.72); 7.5534 (4.74); 7.5518 (4.57); 7.5414 (4.6); 7.5396 (4.43); 7.0696 (13.27);
7.0628 (13.12); 5.7569 (13.74); 4.1008 (0.98); 4.0876 (1); 4.0745 (0.34); 4.0207 (0.34); 3.3247 (94.14); 3.1761
(4.4); 3.163 (4.27); 2.6767 (0.66); 2.672 (0.9); 2.6675 (0.64); 2.5254 (2.78); 2.5205 (4.37); 2.512 (51.18); 2.5075
(102.26); 2.5029 (134.94); 2.4984 (97.18); 2.4939 (46.08); 2.3342 (0.64); 2.3298 (0.89); 2.3251 (0.65); 1.9894
(1.46); 1.1932 (0.39); 1.1753 (0.79); 1.1575 (0.39); 0.1461 (0.37); 0.008 (3.11); −0.0002 (86.72); −0.0085 (2.62); −0.1494
(0.38)
Compound No. I-1-32, solvent: [DMSO], spectrometer: 399.95 MHz
10.9793 (1.72); 9.111 (1.5); 9.1062 (1.51); 8.7277 (1.14); 8.7245 (1.21); 8.7158 (1.21); 8.7125 (1.2); 8.55 (1.74);
8.3219 (0.89); 8.3172 (0.58); 8.3061 (0.67); 8.302 (0.93); 8.2973 (0.58); 7.533 (0.9); 7.5211 (0.94); 7.5131 (1.04);
7.5012 (0.89); 6.4696 (2.23); 5.7575 (0.45); 4.6696 (4.7); 4.5295 (0.37); 3.8833 (0.69); 3.7057 (1.43); 3.6082
(0.33); 3.5968 (2.19); 3.5641 (10.33); 3.5535 (1.5); 3.5465 (0.5); 3.3379 (1.67); 2.5075 (8.7); 2.5035 (10.77);
2.4992 (7.83); 2.2016 (8.09); 2.0567 (0.63); 1.9896 (0.69); 1.4754 (3.67); 1.3772 (16); 1.363 (3.13); 1.3502 (2.31);
1.1748 (0.42); 0.0004 (0.78); −0.0002 (0.79)
Compound No. I-1-115, solvent: [DMSO], spectrometer: 399.95 MHz
10.9991 (3.37); 9.1096 (3.07); 9.1053 (3.13); 8.7284 (2.1); 8.7249 (2.22); 8.7166 (2.21); 8.7131 (2.15); 8.3224
(1.7); 8.3023 (1.79); 7.5338 (1.6); 7.5219 (1.61); 7.5142 (1.61); 7.502 (1.52); 6.4914 (4.6); 4.8147 (10.11); 3.3329
(9.41); 2.5023 (38.1); 2.2254 (16); 2.0742 (0.34); 1.2351 (0.33); −0.0002 (1.84)
Compound No. I-1-24, solvent: [DMSO], spectrometer: 601.6 MHz
11.0424 (1.93); 9.1088 (1.64); 9.1059 (1.6); 8.7305 (1.4); 8.7279 (1.49); 8.7226 (1.45); 8.7199 (1.48); 8.323
(0.66); 8.3197 (0.95); 8.3165 (0.69); 8.3098 (0.71); 8.3063 (1); 8.3033 (0.69); 7.5336 (0.97); 7.5325 (1.08); 7.5257
(0.95); 7.5245 (1.01); 7.5205 (0.94); 7.5193 (0.99); 7.5125 (0.92); 7.5113 (0.97); 6.517 (2.49); 4.9386 (7.38);
4.6393 (0.77); 4.1777 (1.25); 4.1659 (3.99); 4.1541 (4.02); 4.1422 (1.29); 4.1179 (0.43); 4.1061 (0.43); 4.046
(1.2); 4.0342 (3.63); 4.0224 (3.66); 4.0105 (1.21); 3.3638 (26.71); 2.511 (3.92); 2.508 (8.39); 2.505 (11.54); 2.502
(8.39); 2.499 (3.84); 2.234 (10.13); 2.0586 (0.92); 2.0578 (0.96); 1.9957 (0.34); 1.9911 (16); 1.3963 (0.42); 1.225
(4.65); 1.2132 (9.8); 1.2014 (4.64); 1.1917 (0.95); 1.1865 (4.54); 1.1799 (0.54); 1.1746 (8.86); 1.1628 (4.5)
Compound No. I-1-41, solvent: [DMSO], spectrometer: 601.6 MHz
11.1038 (1.55); 8.9982 (0.86); 8.9956 (1.49); 8.9932 (0.84); 8.7657 (1.62); 8.7611 (1.61); 8.5651 (1.44); 8.243
(0.47); 8.2401 (0.54); 8.2385 (0.54); 8.2356 (0.45); 8.227 (0.49); 8.2241 (0.57); 8.2225 (0.53); 8.2195 (0.44);
6.4699 (1.85); 4.6717 (4.09); 3.6936 (0.46); 3.5603 (13.53); 3.3484 (9.25); 2.5424 (3.35); 2.509 (7.78); 2.506
(17.41); 2.503 (23.98); 2.4999 (17.12); 2.4969 (7.53); 2.2017 (6.79); 2.2012 (6.52); 2.077 (0.43); 1.3731 (16); −0.0002
(6.15)
Compound No. I-1-7 (Synthesis Example 12), solvent: [DMSO], spectrometer: 399.95 MHz
11.0604 (2.71); 8.9829 (1.96); 8.9791 (3.18); 8.7886 (8.34); 8.7764 (8.39); 8.7417 (3.2); 8.7348 (3.23); 8.2252
(1.03); 8.2207 (1.24); 8.2185 (1.24); 8.2141 (1.01); 8.201 (1.05); 8.1965 (1.24); 8.1943 (1.18); 8.1898 (0.93);
7.4498 (1.94); 7.4375 (3.71); 7.4253 (1.83); 6.5223 (3.92); 5.4262 (10.31); 4.0391 (0.55); 4.0213 (0.54); 3.5678
(0.39); 3.4225 (0.4); 3.3117 (570.27); 2.6744 (0.43); 2.6697 (0.6); 2.6652 (0.44); 2.5397 (0.8); 2.5228 (2.6);
2.5095 (33.79); 2.5051 (63.13); 2.5007 (82.71); 2.4962 (57.45); 2.4919 (27.5); 2.3215 (16); 1.9868 (2.34); 1.2363
(0.64); 1.1928 (0.67); 1.1749 (1.3); 1.1572 (0.65); −0.0002 (0.58)
Compound No. I-1-29: mass (m/z): 354.1 (M + H)⁺; 1H NMR (DMSO-D₆) 0.60-0.64 (m, 2H), 0.94-0.98 (m,
2H), 1.90-2.00 (m, 1H), 5.40 (s, 2H), 6.33 (s, 1H), 7.46-7.50 (m, 2H), 7.62-7.65 (m, 1H), 8.26-8.32 (m, 2H),
8.68-8.70 (m, 1H), 9.08 (s, 1H), 10.80 (s, 1H).
Compound No. I-1-61: HPLC-MS: logP = 1.35; mass (m/z): 378.1 (M + H)⁺; 1H NMR (CD₃CN) 1.21-1.28 (m,
3H), 2.18 (s, 3H), 2.89 und 3.09 (s, 3H), 4.05 (s, 2H), 4.12-4.23 (m, 2H), 4.78 und 4.93 (s, 2H), 6.53 (s, 1H),
7.98-8.00 (m, 1H), 8.61-8.62 (m, 1H), 8.90 (s, 1H), 9.19 (s. 1H); 13C NMR (CD₃CN) 14.4, 35.3, 36.1, 50.5, 51.1,
51.3, 61.8, 62.4, 98.6, 122.9, 123.0, 132.6, 141.4, 141.9, 145.7, 146.6, 159.2, 160.9, 162.8, 168.3, 168.4, 170.1.
Compound No. I-1-114, solvent: [DMSO], spectrometer: 399.95 MHz
11.079 (3.12); 8.9949 (3.4); 8.7564 (3.21); 8.7496 (3.28); 8.2412 (1.09); 8.235 (1.37); 8.2304 (1.09); 8.2171
(1.13); 8.2126 (1.38); 8.2062 (1.05); 6.487 (4.2); 4.8053 (10.5); 3.4508 (2.35); 3.4331 (2.9); 3.3269 (16.37);
3.0754 (0.46); 2.6749 (0.39); 2.6703 (0.46); 2.6657 (0.38); 2.54 (1.04); 2.5056 (41.86); 2.5013 (52.51); 2.497
(37.41); 2.3279 (0.37); 2.2264 (16); 2.0691 (0.57); 1.2372 (0.98); 1.1696 (0.38); 1.0745 (0.71); 1.0571 (1.35);
1.0396 (0.68); 0.8749 (0.46); −0.0002 (2.39)
Compound No. I-1-59, solvent: [DMSO], spectrometer: 399.95 MHz
11.0906 (2.78); 8.9963 (2.03); 8.9925 (3.29); 8.9362 (0.34); 8.7579 (3.28); 8.751 (3.32); 8.2406 (1.09); 8.2361
(1.3); 8.2339 (1.3); 8.2294 (1.07); 8.2164 (1.11); 8.212 (1.33); 8.2099 (1.25); 8.2053 (1.02); 6.5088 (3.89); 4.931
(11.68); 4.7731 (0.36); 4.4845 (0.49); 4.1869 (1.95); 4.1691 (6.04); 4.1514 (6.08); 4.1419 (0.5); 4.1336 (2.02);
3.376 (0.39); 3.3599 (0.69); 3.3019 (305.24); 2.6736 (0.56); 2.669 (0.74); 2.6645 (0.55); 2.539 (1.16); 2.5221
(3.62); 2.5088 (43.33); 2.5044 (80.04); 2.5 (104.25); 2.4956 (73.63); 2.4912 (36.16); 2.3313 (0.62); 2.3268 (0.78);
2.3221 (0.59); 2.3179 (0.34); 2.2358 (16); 2.1814 (0.38); 2.0695 (0.76); 2.0591 (0.71); 1.9913 (1.03); 1.4627
(0.34); 1.3561 (1.3); 1.3308 (0.42); 1.3118 (0.4); 1.2985 (0.34); 1.2591 (0.34); 1.2299 (6.95); 1.2121 (14.17);
1.1943 (6.7); 1.1811 (0.41); 1.1413 (0.35); 0.8932 (0.68); 0.8855 (0.7); 0.8787 (0.65); 0.8748 (1.28); 0.8691
(0.77); 0.8565 (0.62); 0.8507 (0.38); 0.845 (0.46); 0.8281 (0.41); 0.0079 (0.95); −0.0002 (17.7); −0.0084 (0.82)
Compound No. I-1-47, solvent: [DMSO], spectrometer: 601.6 MHz
10.5679 (1.36); 9.1121 (1.36); 8.7939 (1.41); 8.7913 (1.48); 8.7859 (1.48); 8.7833 (1.44); 8.3051 (0.76); 8.292
(0.79); 7.5925 (1.05); 7.5844 (1.05); 7.5793 (1.06); 7.5713 (0.99); 7.4676 (1.25); 7.3493 (2.86); 7.231 (1.42);
5.7622 (6.17); 3.7493 (16); 3.3451 (39.63); 3.3215 (0.52); 2.5214 (0.33); 2.5183 (0.32); 2.5094 (7.19); 2.5064
(15.55); 2.5034 (21.3); 2.5003 (15.57); 2.4974 (7.19); −0.0002 (2.4)
Compound No. I-1-113, solvent: [DMSO], spectrometer: 399.95 MHz
10.6385 (2.6); 8.9971 (2.07); 8.8308 (2.66); 8.8238 (2.62); 8.1996 (0.77); 8.1952 (0.96); 8.1891 (0.74); 8.1762
(0.8); 8.1717 (0.95); 7.5153 (1.6); 7.3372 (3.51); 7.1592 (1.74); 5.7471 (0.37); 3.7498 (16); 3.307 (150.9); 3.2851
(1.15); 2.5397 (0.41); 2.5228 (1.23); 2.5095 (13.33); 2.5052 (23.9); 2.5007 (30.52); 2.4963 (20.91); 2.4919 (9.89)
Compound No. I-1-112 (Synthesis Example 5), solvent: [DMSO], spectrometer: 399.95 MHz
11.2317 (1.77); 8.9941 (1.43); 8.9902 (2.41); 8.9863 (1.34); 8.7756 (2.19); 8.7687 (2.22); 8.2396 (0.82); 8.2352
(0.95); 8.2328 (0.94); 8.2283 (0.79); 8.2156 (0.84); 8.2111 (0.96); 8.2087 (0.9); 8.2043 (0.76); 7.5178 (1.32);
7.3367 (2.89); 7.1557 (1.42); 6.4514 (2.93); 6.2668 (0.34); 3.636 (16); 3.5669 (1.96); 3.3175 (165.02); 2.5243
(0.62); 2.511 (8.63); 2.5066 (16.04); 2.5021 (20.89); 2.4977 (14.52); 2.4933 (6.95); 2.0702 (0.72); −0.0002 (1.46)
Compound No. I-1-111, solvent: [DMSO], spectrometer: 399.95 MHz
11.1283 (2.55); 9.1083 (2.89); 9.1034 (2.46); 8.7443 (2.1); 8.7404 (2.08); 8.7324 (1.86); 8.7285 (1.56); 8.3235
(1.59); 8.3191 (1.86); 8.3143 (1.36); 8.3037 (1.45); 8.2992 (1.63); 8.2946 (0.98); 7.5475 (1.78); 7.5353 (1.9);
7.5276 (1.76); 7.5164 (2.41); 7.3354 (2.76); 7.1543 (1.37); 6.4533 (3.65); 3.6312 (16); 3.3104 (29.99); 2.5661
(1.17); 2.5054 (25.89); 2.5012 (27.09); −0.0002 (2.17)
Compound No. I-3-1 (Synthesis Example 15), solvent: [DMSO], spectrometer: 399.95 MHz
11.1114 (2.03); 9.1905 (1.95); 9.185 (1.88); 8.8172 (1.43); 8.8131 (1.59); 8.8051 (1.53); 8.8011 (1.55); 8.5571
(1.18); 8.5546 (1.25); 8.5526 (1.18); 8.545 (1.26); 8.5425 (1.22); 8.5405 (1.15); 8.3894 (0.76); 8.3841 (1.12);
8.3796 (0.79); 8.3695 (0.82); 8.3641 (1.19); 8.3596 (0.8); 8.0746 (0.68); 8.07 (0.69); 8.054 (1.22); 8.0518 (1.1);
8.0495 (1.19); 8.0355 (1.05); 8.0309 (1.04); 7.9562 (2.21); 7.9356 (1.54); 7.6206 (1.12); 7.6085 (1.12); 7.6007
(1.09); 7.5902 (0.93); 7.5886 (1.04); 7.4773 (0.96); 7.475 (1.09); 7.4652 (0.95); 7.4629 (1.12); 7.459 (1.03);
7.4566 (1.01); 7.4469 (0.92); 7.4445 (0.98); 3.3248 (20.52); 2.5113 (14.15); 2.507 (28.39); 2.5025 (37.64); 2.498
(27.43); 2.4935 (13.49); 2.3237 (16); −0.0002 (1.69)
Compound No. I-1-22, solvent: [DMSO], spectrometer: 399.95 MHz
8.968 (3.4); 8.962 (3.6); 8.806 (2.3); 8.803 (2.5); 8.794 (2.4); 8.790 (2.3); 8.177 (1.3); 8.173 (2.0); 8.168 (1.4);
8.158 (1.5); 8.153 (2.1); 8.148 (1.4); 7.907 (1.0); 7.903 (0.8); 7.862 (4.0); 7.858 (2.8); 7.841 (0.6); 7.822 (0.6);
7.782 (2.2); 7.763 (2.6); 7.744 (1.7); 7.741 (1.7); 7.726 (2.1); 7.723 (2.1); 7.721 (2.2); 7.694 (0.4); 7.679 (1.9);
7.676 (1.9); 7.661 (1.9); 7.659 (2.0); 7.656 (2.2); 7.609 (2.3); 7.601 (3.1); 7.597 (2.6); 7.589 (2.5); 7.581 (4.2);
7.561 (1.7); 7.548 (0.4); 7.527 (0.6); 7.513 (0.8); 7.494 (0.9); 7.474 (0.4); 7.400 (2.5); 7.396 (3.9); 7.392 (2.6);
7.343 (2.1); 7.323 (3.9); 7.303 (2.0); 6.941 (2.4); 6.921 (2.2); 6.748 (5.2); 6.746 (4.8); 5.756 (1.6); 4.056 (0.8);
4.038 (2.4); 4.020 (2.4); 4.003 (0.8); 3.323 (64.9); 2.671 (1.0); 2.667 (0.8); 2.651 (0.8); 2.614 (16.0); 2.541 (1.1);
2.507 (110.7); 2.502 (141.1); 2.498 (105.0); 2.334 (0.7); 2.329 (0.9); 2.325 (0.7); 1.989 (10.1); 1.398 (0.9); 1.299
(0.6); 1.259 (0.9); 1.235 (2.4); 1.193 (2.9); 1.175 (5.7); 1.157 (2.8); 0.868 (0.3); 0.854 (0.4); 0.146 (0.7); 0.008
(7.7); 0.000 (145.2); −0.008 (6.0); −0.149 (0.7)
Compound No. I-1-120, solvent: [DMSO], spectrometer: 399.95 MHz
11.435 (1.5); 9.136 (1.7); 9.132 (1.7); 9.130 (1.6); 8.763 (1.3); 8.759 (1.4); 8.751 (1.3); 8.747 (1.3); 8.358 (0.8);
8.352 (1.0); 8.348 (0.8); 8.338 (0.9); 8.332 (1.1); 8.328 (0.8); 7.561 (0.9); 7.559 (1.0); 7.549 (0.9); 7.547 (1.0);
7.541 (0.9); 7.539 (0.9); 7.529 (0.9); 7.527 (0.9); 6.861 (2.4); 6.859 (2.3); 3.322 (11.6); 3.176 (0.6); 3.163 (0.6);
2.608 (16.0); 2.544 (8.9); 2.542 (8.9); 2.525 (0.5); 2.511 (10.1); 2.507 (20.0); 2.502 (25.9); 2.498 (18.3); 2.493
(8.5); 0.008 (1.2); 0.000 (28.9); −0.009 (0.9)
Compound No. I-1-42, solvent: [DMSO], spectrometer: 399.95 MHz
13.176 (0.6); 11.214 (1.5); 8.962 (3.1); 8.957 (3.1); 8.923 (1.3); 8.919 (1.2); 8.816 (2.1); 8.812 (2.3); 8.803 (2.3);
8.800 (2.2); 8.746 (0.9); 8.742 (1.0); 8.734 (1.0); 8.730 (0.9); 8.184 (1.4); 8.178 (2.0); 8.174 (1.5); 8.164 (1.6);
8.158 (2.2); 8.154 (1.5); 8.141 (0.6); 8.136 (0.9); 8.131 (0.6); 8.121 (0.7); 8.115 (0.9); 8.111 (0.6); 8.006 (0.4);
7.985 (0.4); 7.959 (0.5); 7.953 (3.4); 7.948 (1.5); 7.941 (1.6); 7.937 (2.0); 7.931 (4.1); 7.926 (0.7); 7.911 (1.4);
7.869 (1.2); 7.863 (8.8); 7.858 (3.0); 7.846 (3.5); 7.841 (11.2); 7.835 (1.5); 7.809 (0.4); 7.803 (2.7); 7.798 (1.0);
7.786 (1.1); 7.781 (3.4); 7.775 (0.5); 7.681 (1.2); 7.675 (8.2); 7.670 (2.5); 7.666 (0.9); 7.658 (2.5); 7.653 (7.1);
7.649 (4.3); 7.644 (1.7); 7.632 (1.2); 7.627 (4.6); 7.613 (2.0); 7.606 (2.2); 7.594 (1.8); 7.593 (1.9); 7.587 (0.7);
7.581 (3.9); 7.576 (1.7); 7.570 (3.5); 7.564 (2.0); 7.559 (3.6); 7.553 (1.4); 7.548 (3.0); 7.539 (1.1); 7.526 (1.0);
7.518 (1.0); 7.506 (0.8); 7.399 (0.9); 7.393 (6.7); 7.389 (2.3); 7.376 (2.4); 7.372 (8.2); 7.366 (1.1); 7.125 (1.0);
7.119 (7.1); 7.114 (2.3); 7.102 (2.0); 7.097 (6.2); 7.091 (0.8); 6.801 (0.4); 6.766 (3.8); 6.709 (5.4); 6.707 (5.5);
5.757 (13.3); 5.367 (1.4); 5.337 (1.4); 3.522 (1.4); 3.504 (4.2); 3.486 (4.3); 3.469 (1.4); 3.395 (1.3); 3.377 (3.9);
3.359 (4.0); 3.341 (1.7); 3.326 (28.8); 2.677 (0.4); 2.672 (0.5); 2.668 (0.4); 2.646 (0.8); 2.595 (16.0); 2.526 (1.5);
2.512 (28.6); 2.508 (57.0); 2.503 (74.5); 2.499 (53.1); 2.494 (25.1); 2.335 (0.4); 2.330 (0.5); 2.326 (0.4); 2.245
(1.2); 2.163 (8.7); 1.836 (0.4); 1.642 (0.4); 1.299 (0.4); 1.259 (1.0); 1.234 (3.3); 1.214 (0.8); 1.194 (5.7); 1.176
(11.6); 1.158 (5.5); 1.136 (5.1); 1.118 (10.8); 1.100 (4.9); 0.853 (0.4); 0.146 (0.4); 0.008 (3.9); 0.000 (99.1); −0.009
(3.4); −0.150 (0.5)
Compound No. I-1-19: mass (m/z): 241.1 (M + H)⁺; 1H NMR (DMSO-D₆) 2.31 (s, 3H), 3.27-3.28 (m, 1H),
4.86 (s, 2H), 6.47 (s, 1H), 7.47-7.51 (m, 1H), 8.28-8.31 (m, 1H), 8.69-8.71 (m, 1H), 9.10 (s, 1H), 10.80 (s, 1H).
Compound No. I-1-75, solvent: [DMSO], spectrometer: 399.95 MHz
11.105 (0.6); 9.100 (0.6); 9.096 (0.6); 8.719 (0.5); 8.715 (0.5); 8.707 (0.5); 8.703 (0.5); 8.314 (0.3); 8.294 (0.4);
7.822 (0.9); 7.816 (0.9); 7.523 (0.3); 7.521 (0.3); 7.511 (0.3); 7.509 (0.3); 7.503 (0.3); 7.501 (0.3); 7.489 (0.3);
7.375 (1.1); 7.371 (0.4); 7.359 (0.4); 7.354 (1.4); 7.198 (1.1); 7.177 (0.9); 6.661 (0.7); 6.655 (0.7); 5.220 (2.0);
3.331 (6.1); 2.541 (8.0); 2.520 (0.5); 2.511 (7.6); 2.507 (15.8); 2.502 (21.2); 2.497 (15.5); 2.493 (7.5); 1.254
(16.0); 0.000 (6.0)
Compound No. I-1-86, solvent: [DMSO], spectrometer: 399.95 MHz
11.192 (0.6); 8.940 (0.6); 8.934 (0.6); 8.359 (0.4); 8.352 (0.4); 8.338 (0.4); 8.331 (0.4); 7.829 (0.8); 7.823 (0.8);
7.659 (0.8); 7.638 (0.6); 7.373 (1.0); 7.357 (0.4); 7.352 (1.3); 7.194 (1.0); 7.174 (0.8); 6.651 (0.7); 6.646 (0.7);
5.219 (1.9); 3.330 (41.1); 2.542 (7.5); 2.524 (0.5); 2.511 (9.8); 2.507 (19.6); 2.502 (25.9); 2.498 (18.8); 2.493
(9.1); 1.290 (0.8); 1.273 (1.0); 1.267 (1.2); 1.253 (16.0); 0.000 (6.9)
Compound No. I-1-70, solvent: [DMSO], spectrometer: 399.95 MHz
11.129 (4.3); 9.105 (4.4); 9.101 (4.3); 8.724 (3.7); 8.720 (3.9); 8.712 (3.9); 8.708 (3.9); 8.324 (1.8); 8.320 (2.6);
8.315 (1.8); 8.304 (2.0); 8.299 (2.7); 8.295 (1.8); 7.865 (6.9); 7.859 (6.8); 7.528 (2.6); 7.526 (2.7); 7.516 (2.6);
7.514 (2.6); 7.508 (2.5); 7.506 (2.6); 7.496 (2.5); 7.494 (2.5); 7.432 (1.5); 7.416 (1.7); 7.411 (3.2); 7.395 (2.9);
7.391 (1.9); 7.376 (1.9); 7.156 (1.2); 7.149 (1.3); 7.133 (2.0); 7.128 (2.2); 7.112 (1.0); 7.108 (1.1); 7.106 (1.1);
7.084 (3.4); 7.075 (1.9); 7.065 (5.0); 7.049 (1.9); 7.044 (2.0); 6.696 (5.6); 6.690 (5.5); 5.307 (16.0); 3.330 (211.9);
2.996 (0.3); 2.676 (0.6); 2.671 (0.8); 2.667 (0.6); 2.541 (42.5); 2.524 (1.8); 2.520 (2.8); 2.511 (44.3); 2.507 (90.4);
2.502 (120.2); 2.497 (86.8); 2.493 (41.2); 2.333 (0.6); 2.329 (0.8); 2.324 (0.6); 1.273 (0.4); 1.258 (0.6); 1.242
(0.8); 0.008 (1.1); 0.000 (35.0); −0.009 (1.1)
Compound No. I-1-74, solvent: [DMSO], spectrometer: 399.95 MHz
11.118 (1.7); 9.105 (1.6); 9.100 (1.6); 8.722 (1.2); 8.718 (1.3); 8.710 (1.3); 8.706 (1.3); 8.324 (0.6); 8.319 (1.0);
8.314 (0.7); 8.304 (0.7); 8.299 (1.0); 8.294 (0.7); 7.831 (2.5); 7.825 (2.4); 7.524 (1.0); 7.512 (1.0); 7.505 (1.0);
7.492 (0.9); 7.283 (1.0); 7.264 (1.7); 7.244 (1.1); 6.876 (0.8); 6.870 (1.0); 6.856 (0.7); 6.849 (1.0); 6.818 (1.9);
6.813 (2.7); 6.794 (1.1); 6.676 (2.0); 6.671 (2.0); 5.239 (5.8); 3.728 (16.0); 3.330 (109.7); 3.308 (0.4); 2.541
(12.8); 2.524 (0.9); 2.511 (18.3); 2.506 (36.6); 2.502 (48.4); 2.497 (35.4); 2.493 (17.1); 0.000 (8.7)
Compound No. I-1-76, solvent: [DMSO], spectrometer: 399.95 MHz
11.125 (5.5); 9.095 (5.4); 9.091 (5.3); 9.076 (2.8); 9.074 (3.0); 9.071 (2.9); 9.069 (2.8); 8.800 (2.3); 8.796 (2.4);
8.788 (2.4); 8.784 (2.4); 8.720 (4.2); 8.716 (4.5); 8.708 (4.5); 8.704 (4.4); 8.317 (2.2); 8.312 (3.2); 8.307 (2.2);
8.297 (2.4); 8.292 (3.4); 8.287 (2.5); 8.285 (2.4); 8.280 (2.4); 8.275 (1.5); 8.265 (1.6); 8.260 (2.4); 8.255 (1.5);
7.823 (7.4); 7.817 (7.4); 7.567 (1.8); 7.565 (1.9); 7.554 (1.8); 7.553 (1.9); 7.547 (1.8); 7.545 (1.8); 7.535 (1.7);
7.533 (1.8); 7.522 (3.1); 7.521 (3.2); 7.509 (3.1); 7.503 (3.0); 7.501 (3.1); 7.490 (2.9); 7.489 (2.9); 7.359 (1.4);
7.342 (1.8); 7.338 (3.0); 7.321 (3.1); 7.316 (2.1); 7.311 (2.1); 7.304 (2.2); 7.300 (1.9); 7.287 (2.3); 7.285 (2.5);
7.281 (2.6); 7.279 (2.4); 7.261 (1.8); 7.255 (1.9); 7.127 (1.6); 7.121 (1.5); 7.106 (2.9); 7.101 (2.9); 7.085 (1.3);
7.080 (1.2); 6.677 (6.7); 6.671 (6.6); 5.315 (16.0); 3.621 (0.4); 3.611 (0.4); 3.333 (271.4); 3.153 (0.5); 3.143 (0.5);
3.135 (0.5); 3.124 (0.5); 2.676 (0.9); 2.671 (1.3); 2.667 (1.0); 2.662 (0.5); 2.542 (9.9); 2.525 (3.1); 2.511 (71.7);
2.507 (146.0); 2.502 (193.8); 2.498 (141.6); 2.493 (68.5); 2.338 (0.5); 2.333 (0.9); 2.329 (1.3); 2.324 (1.0); 1.278
(3.5); 1.261 (6.9); 1.245 (4.1); 1.230 (1.1); 1.215 (1.1); 1.199 (1.1); 1.179 (0.4); 0.008 (1.6); 0.000 (51.9); −0.009
(1.8)
Compound No. I-1-71, solvent: [DMSO], spectrometer: 399.95 MHz
11.133 (4.7); 9.099 (4.8); 9.095 (4.8); 8.722 (3.8); 8.718 (4.1); 8.710 (4.0); 8.706 (4.0); 8.319 (2.0); 8.314 (2.8);
8.309 (2.0); 8.299 (2.1); 8.294 (2.9); 8.289 (2.0); 7.908 (7.3); 7.902 (7.3); 7.687 (2.0); 7.668 (3.6); 7.628 (7.2);
7.609 (4.3); 7.590 (2.3); 7.546 (3.6); 7.524 (4.4); 7.522 (3.9); 7.512 (2.8); 7.510 (2.8); 7.504 (2.7); 7.502 (2.7);
7.492 (2.6); 7.490 (2.6); 6.708 (5.9); 6.702 (5.9); 5.399 (16.0); 3.390 (0.3); 3.331 (354.8); 2.996 (2.3); 2.676 (0.8);
2.671 (1.0); 2.667 (0.8); 2.662 (0.4); 2.541 (28.4); 2.524 (3.5); 2.511 (57.0); 2.507 (114.1); 2.502 (151.3); 2.498
(111.3); 2.493 (54.4); 2.338 (0.3); 2.333 (0.7); 2.329 (1.0); 2.324 (0.7); 1.266 (0.8); 1.259 (0.3); 1.237 (1.3); 0.008
(1.1); 0.000 (29.0); −0.009 (1.0)
Compound No. I-1-87, solvent: [DMSO], spectrometer: 399.95 MHz
11.216 (5.3); 8.938 (5.0); 8.933 (5.0); 8.890 (0.5); 8.885 (0.5); 8.359 (3.4); 8.353 (3.3); 8.338 (3.6); 8.332 (3.5);
8.302 (0.5); 8.296 (0.5); 8.281 (0.5); 8.275 (0.5); 7.913 (7.5); 7.908 (7.4); 7.687 (2.0); 7.682 (1.7); 7.668 (3.8);
7.660 (7.0); 7.639 (5.7); 7.638 (5.6); 7.627 (7.1); 7.607 (4.4); 7.589 (2.5); 7.544 (3.5); 7.524 (2.2); 6.698 (6.2);
6.692 (6.1); 5.399 (16.0); 5.318 (0.6); 3.642 (0.3); 3.636 (0.6); 3.626 (0.7); 3.620 (0.8); 3.610 (0.8); 3.604 (0.6);
3.593 (0.6); 3.390 (0.4); 3.332 (532.2); 3.292 (0.6); 3.282 (0.4); 3.169 (0.4); 3.159 (0.4); 3.151 (1.0); 3.141 (1.0);
3.133 (1.0); 3.122 (1.1); 3.115 (0.5); 3.104 (0.5); 2.996 (0.7); 2.922 (0.4); 2.676 (1.0); 2.671 (1.3); 2.667 (1.0);
2.662 (0.5); 2.561 (0.5); 2.542 (50.9); 2.525 (3.3); 2.520 (5.1); 2.511 (72.2); 2.507 (147.1); 2.502 (195.0); 2.498
(140.8); 2.493 (66.7); 2.338 (0.4); 2.334 (0.9); 2.329 (1.3); 2.324 (0.9); 1.427 (0.4); 1.281 (7.4); 1.263 (13.2);
1.251 (6.3); 1.246 (8.7); 1.233 (2.7); 1.218 (1.5); 1.211 (0.4); 1.201 (1.6); 1.180 (1.7); 1.162 (0.9); 1.036 (1.3);
1.019 (1.3); 0.008 (1.4); 0.000 (45.4); −0.009 (1.4)
Compound No. I-1-77, solvent: [DMSO], spectrometer: 399.95 MHz
11.134 (1.6); 9.099 (1.6); 9.095 (1.6); 8.720 (1.3); 8.716 (1.4); 8.708 (1.4); 8.704 (1.3); 8.319 (0.6); 8.314 (0.9);
8.309 (0.6); 8.299 (0.7); 8.294 (1.0); 8.289 (0.6); 7.905 (0.6); 7.901 (1.1); 7.893 (2.7); 7.888 (3.0); 7.879 (1.0);
7.869 (1.9); 7.556 (0.4); 7.542 (1.4); 7.538 (3.4); 7.520 (2.5); 7.510 (1.0); 7.509 (1.0); 7.502 (1.3); 7.501 (1.3);
7.490 (0.9); 7.489 (0.9); 6.698 (2.0); 6.692 (2.0); 5.369 (5.4); 3.846 (16.0); 3.327 (16.1); 2.671 (0.4); 2.541 (21.6);
2.524 (0.8); 2.520 (1.3); 2.511 (20.8); 2.507 (42.7); 2.502 (57.0); 2.497 (41.3); 2.493 (19.7); 2.329 (0.4); 0.008
(0.5); 0.000 (16.3); −0.009 (0.5)
Compound No. I-1-88, solvent: [DMSO], spectrometer: 399.95 MHz
11.127 (3.8); 9.097 (5.0); 9.093 (5.0); 8.717 (4.0); 8.713 (4.2); 8.705 (4.1); 8.701 (4.0); 8.318 (4.1); 8.312 (3.0);
8.307 (2.0); 8.297 (2.2); 8.292 (3.1); 8.287 (2.0); 7.826 (7.2); 7.820 (7.2); 7.519 (3.0); 7.517 (2.8); 7.507 (2.9);
7.505 (2.8); 7.499 (2.9); 7.487 (2.7); 7.485 (2.6); 7.406 (0.8); 7.401 (0.8); 7.392 (0.9); 7.386 (1.9); 7.381 (1.3);
7.376 (1.1); 7.367 (2.0); 7.363 (1.4); 7.354 (1.0); 7.349 (1.2); 7.256 (1.1); 7.251 (3.4); 7.238 (2.9); 7.231 (3.5);
7.227 (2.8); 7.222 (4.4); 7.212 (5.3); 7.202 (2.3); 7.195 (4.1); 7.192 (3.8); 7.176 (1.4); 7.173 (1.2); 6.683 (6.8);
6.678 (6.7); 5.341 (16.0); 3.327 (127.8); 3.304 (1.6); 2.675 (0.7); 2.671 (0.9); 2.666 (0.7); 2.541 (10.7); 2.524
(2.3); 2.519 (3.5); 2.511 (50.7); 2.506 (103.6); 2.502 (137.7); 2.497 (99.4); 2.493 (47.3); 2.333 (0.7); 2.329 (0.9);
2.324 (0.6); 1.239 (0.6); 0.008 (1.4); 0.000 (45.2); −0.009 (1.5)
Compound No. I-1-89, solvent: [DMSO], spectrometer: 399.95 MHz
11.143 (4.4); 9.095 (4.1); 9.090 (4.0); 8.719 (3.1); 8.715 (3.2); 8.707 (3.3); 8.703 (3.1); 8.316 (1.8); 8.311 (2.6);
8.306 (1.7); 8.296 (1.9); 8.291 (2.7); 8.286 (1.7); 7.898 (9.4); 7.892 (6.8); 7.879 (3.6); 7.876 (3.6); 7.732 (1.8);
7.729 (1.7); 7.713 (3.7); 7.710 (3.5); 7.694 (2.3); 7.691 (2.1); 7.547 (2.4); 7.545 (2.4); 7.528 (3.9); 7.526 (3.9);
7.520 (2.7); 7.507 (4.1); 7.500 (2.5); 7.488 (2.3); 7.305 (3.6); 7.285 (3.3); 6.727 (5.4); 6.722 (5.3); 5.501 (16.0);
3.328 (246.4); 2.995 (1.1); 2.675 (0.9); 2.671 (1.2); 2.666 (0.9); 2.560 (0.4); 2.541 (17.6); 2.524 (3.0); 2.511
(69.5); 2.506 (140.1); 2.502 (184.9); 2.497 (134.0); 2.493 (64.4); 2.338 (0.4); 2.333 (0.9); 2.329 (1.2); 2.324 (0.8);
2.320 (0.5); 1.388 (0.4); 1.276 (2.3); 1.266 (1.9); 1.260 (4.5); 1.244 (3.6); 1.237 (2.7); 1.228 (1.6); 1.213 (0.5);
1.196 (0.5); 1.177 (0.3); 1.031 (0.4); 1.014 (0.4); 0.853 (0.5); 0.008 (1.4); 0.000 (48.3); −0.009 (1.6)
Compound No. I-1-90, solvent: [DMSO], spectrometer: 399.95 MHz
11.128 (5.2); 9.100 (5.1); 9.095 (5.2); 9.074 (0.4); 9.071 (0.4); 8.796 (0.3); 8.788 (0.4); 8.784 (0.3); 8.723 (4.2);
8.719 (4.5); 8.711 (4.4); 8.707 (4.4); 8.321 (2.2); 8.316 (3.2); 8.311 (2.2); 8.301 (2.3); 8.296 (3.3); 8.291 (2.2);
8.285 (0.4); 8.280 (0.4); 8.260 (0.4); 7.852 (7.5); 7.846 (7.6); 7.533 (0.4); 7.526 (3.1); 7.524 (3.0); 7.514 (3.1);
7.512 (3.0); 7.506 (3.0); 7.504 (2.9); 7.494 (2.9); 7.492 (2.8); 7.333 (1.3); 7.321 (1.4); 7.310 (3.5); 7.298 (3.5);
7.287 (2.5); 7.275 (2.4); 7.261 (1.3); 7.251 (1.8); 7.242 (2.0); 7.231 (2.4); 7.222 (1.7); 7.209 (1.0); 7.200 (0.7);
7.088 (1.5); 7.080 (1.5); 7.074 (1.7); 7.066 (2.8); 7.058 (1.5); 7.052 (1.6); 7.044 (1.3); 6.694 (6.6); 6.688 (6.6);
5.340 (16.0); 3.622 (0.4); 3.613 (0.4); 3.332 (259.5); 3.154 (0.5); 3.144 (0.5); 3.136 (0.5); 3.125 (0.5); 2.996 (0.4);
2.680 (0.4); 2.676 (0.8); 2.671 (1.2); 2.667 (0.8); 2.662 (0.4); 2.542 (27.4); 2.525 (2.8); 2.520 (4.3); 2.511 (62.8);
2.507 (129.4); 2.502 (172.8); 2.498 (125.1); 2.493 (59.7); 2.338 (0.4); 2.333 (0.8); 2.329 (1.2); 2.324 (0.8); 2.320
(0.4); 1.277 (3.5); 1.260 (7.0); 1.244 (4.5); 1.228 (1.1); 1.214 (0.4); 1.197 (0.4); 1.035 (0.6); 1.018 (0.6); 0.853
(0.3); 0.008 (1.4); 0.000 (46.9); −0.008 (1.5)
Compound No. I-1-78, solvent: [DMSO], spectrometer: 399.95 MHz
11.108 (1.8); 9.101 (1.7); 9.097 (1.8); 8.720 (1.2); 8.716 (1.3); 8.708 (1.3); 8.704 (1.2); 8.315 (1.1); 8.300 (0.7);
8.295 (1.1); 8.291 (0.7); 7.785 (2.4); 7.779 (2.4); 7.522 (1.0); 7.510 (1.1); 7.502 (1.0); 7.490 (0.9); 7.228 (2.9);
7.206 (3.3); 6.927 (0.5); 6.919 (3.7); 6.898 (3.4); 6.650 (2.1); 6.645 (2.1); 5.184 (6.3); 5.103 (0.4); 3.729 (16.0);
3.329 (111.3); 2.671 (0.4); 2.541 (3.9); 2.506 (47.6); 2.502 (62.0); 2.497 (46.9); 2.329 (0.4); 1.033 (0.7); 1.016
(0.7); 0.008 (0.4); 0.000 (9.0); −0.008 (0.5)
Compound No. I-1-91, solvent: [DMSO], spectrometer: 399.95 MHz
11.167 (6.9); 9.109 (6.2); 9.104 (6.2); 8.725 (4.9); 8.721 (5.1); 8.713 (5.1); 8.709 (5.0); 8.329 (2.8); 8.324 (4.0);
8.319 (2.8); 8.309 (3.0); 8.304 (4.2); 8.299 (2.8); 7.881 (10.0); 7.875 (10.0); 7.790 (4.7); 7.771 (5.5); 7.675 (2.2);
7.656 (4.9); 7.637 (3.1); 7.545 (3.1); 7.527 (8.5); 7.526 (8.4); 7.515 (4.3); 7.513 (4.4); 7.507 (5.9); 7.506 (5.5);
7.495 (3.9); 7.493 (3.7); 7.204 (0.3); 7.076 (0.4); 6.964 (4.7); 6.945 (4.5); 6.757 (8.6); 6.752 (8.5); 5.507 (16.0);
3.331 (565.4); 2.680 (0.5); 2.676 (1.2); 2.671 (1.6); 2.666 (1.2); 2.662 (0.5); 2.541 (25.4); 2.524 (3.8); 2.520 (6.1);
2.511 (90.5); 2.507 (186.3); 2.502 (248.0); 2.497 (180.2); 2.493 (86.2); 2.338 (0.6); 2.333 (1.2); 2.329 (1.7); 2.324
(1.2); 2.320 (0.6); 1.258 (0.4); 1.239 (1.4); 1.044 (0.4); 1.027 (0.4); 0.853 (0.4); 0.008 (1.9); 0.000 (65.0); −0.009
(2.1)
Compound No. I-1-73, solvent: [DMSO], spectrometer: 399.95 MHz
11.144 (4.7); 9.108 (4.3); 9.103 (4.3); 8.730 (3.4); 8.725 (3.6); 8.717 (3.6); 8.713 (3.5); 8.334 (1.9); 8.329 (2.7);
8.324 (1.9); 8.314 (2.0); 8.309 (2.8); 8.304 (1.9); 7.898 (0.3); 7.888 (7.5); 7.882 (7.6); 7.538 (2.8); 7.536 (2.8);
7.526 (2.9); 7.524 (3.2); 7.521 (3.1); 7.518 (3.4); 7.516 (2.9); 7.503 (4.6); 7.501 (6.0); 7.481 (4.1); 7.313 (2.4);
7.310 (2.3); 7.293 (2.1); 7.290 (2.0); 7.260 (3.6); 7.240 (7.0); 7.206 (0.3); 6.706 (6.1); 6.700 (6.0); 5.354 (16.0);
5.272 (0.5); 3.359 (227.0); 3.314 (1.2); 2.995 (0.4); 2.680 (0.4); 2.676 (0.8); 2.671 (1.2); 2.667 (0.8); 2.662 (0.4);
2.564 (0.5); 2.550 (1.0); 2.542 (35.8); 2.525 (2.8); 2.520 (4.1); 2.511 (61.4); 2.507 (128.1); 2.502 (172.3); 2.498
(124.3); 2.493 (58.8); 2.338 (0.4); 2.334 (0.8); 2.329 (1.1); 2.324 (0.8); 2.320 (0.4); 1.956 (0.4); 1.239 (1.2); 1.039
(0.8); 1.022 (0.8); 0.008 (1.3); 0.000 (44.9); −0.009 (1.4)
Compound No. I-1-92, solvent: [DMSO], spectrometer: 399.95 MHz
11.155 (0.8); 11.132 (4.1); 9.103 (5.6); 9.099 (5.5); 8.946 (2.9); 8.944 (3.1); 8.941 (3.1); 8.939 (2.7); 8.721 (1.3);
8.717 (4.8); 8.713 (4.2); 8.709 (1.6); 8.705 (5.0); 8.701 (4.0); 8.528 (0.8); 8.452 (2.7); 8.448 (2.8); 8.440 (2.8);
8.436 (2.7); 8.326 (2.1); 8.322 (3.0); 8.317 (2.2); 8.306 (2.2); 8.301 (3.1); 8.297 (2.2); 8.097 (1.8); 8.092 (3.1);
8.088 (1.7); 8.078 (1.8); 8.073 (3.2); 8.068 (1.7); 7.953 (2.4); 7.949 (0.8); 7.936 (1.0); 7.932 (2.6); 7.881 (2.0);
7.876 (2.0); 7.821 (9.3); 7.815 (16.0); 7.799 (3.8); 7.795 (10.2); 7.783 (1.8); 7.762 (1.7); 7.521 (3.0); 7.520 (2.7);
7.509 (3.0); 7.507 (2.8); 7.502 (2.9); 7.500 (2.6); 7.489 (2.8); 7.488 (2.5); 7.403 (1.2); 7.398 (1.2); 7.359 (2.0);
7.338 (1.9); 7.281 (2.1); 7.279 (2.1); 7.269 (2.1); 7.267 (2.0); 7.262 (2.0); 7.260 (1.9); 7.250 (1.9); 7.248 (1.8);
7.138 (8.5); 7.118 (7.8); 7.063 (1.5); 7.042 (1.4); 6.711 (1.5); 6.705 (1.5); 6.674 (6.1); 6.668 (5.9); 5.404 (1.4);
5.398 (1.4); 5.382 (3.5); 5.253 (14.1); 4.997 (2.8); 4.534 (1.7); 4.329 (0.8); 4.311 (2.7); 4.293 (2.8); 4.276 (0.9);
3.335 (328.2); 2.996 (3.0); 2.711 (0.5); 2.680 (0.6); 2.676 (1.3); 2.671 (1.8); 2.666 (1.3); 2.662 (0.6); 2.573 (0.4);
2.541 (94.5); 2.524 (4.1); 2.520 (6.3); 2.511 (92.4); 2.506 (190.1); 2.502 (252.4); 2.497 (181.2); 2.493 (85.0);
2.338 (0.5); 2.333 (1.2); 2.329 (1.6); 2.324 (1.2); 2.320 (0.5); 1.595 (0.6); 1.324 (3.2); 1.307 (6.7); 1.289 (3.1);
1.240 (1.2); 1.220 (0.4); 1.090 (2.1); 1.037 (0.4); 1.020 (0.4); 0.944 (1.9); 0.927 (1.8); 0.008 (2.2); 0.000 (79.0); −0.009
(2.6); −0.012 (0.7); −0.150 (0.3)
Compound No. I-1-81, solvent: [DMSO], spectrometer: 399.95 MHz
11.112 (2.7); 9.109 (2.5); 9.104 (2.4); 8.728 (1.8); 8.724 (1.9); 8.716 (1.9); 8.712 (1.8); 8.338 (1.1); 8.333 (1.6);
8.329 (1.1); 8.318 (1.2); 8.313 (1.7); 8.309 (1.1); 7.730 (4.0); 7.724 (4.0); 7.537 (1.6); 7.525 (1.6); 7.517 (1.5);
7.505 (1.4); 7.206 (0.6); 7.087 (1.9); 7.078 (0.8); 7.068 (3.7); 7.028 (2.1); 7.025 (2.2); 7.006 (1.2); 6.950 (0.6);
6.879 (3.1); 6.674 (3.4); 6.668 (3.3); 5.237 (10.2); 3.398 (49.4); 2.995 (0.4); 2.675 (0.7); 2.671 (1.0); 2.666 (0.7);
2.662 (0.4); 2.563 (0.4); 2.541 (40.7); 2.524 (2.5); 2.519 (3.8); 2.510 (56.2); 2.506 (114.7); 2.501 (152.7); 2.497
(110.5); 2.492 (52.9); 2.337 (0.4); 2.333 (0.7); 2.328 (1.0); 2.324 (0.7); 2.320 (0.4); 2.233 (16.0); 2.224 (15.9);
1.634 (0.3); 1.257 (0.3); 1.239 (1.0); 1.036 (0.5); 1.019 (0.4); 0.008 (1.2); 0.000 (40.7); −0.009 (1.3)
Compound No. I-1-141: ¹H NMR (400.0 MHz, d₆-DMSO):
11.374 (2.2); 9.148 (2.2); 9.144 (2.2); 8.816 (1.6); 8.812 (1.6); 8.804 (1.7); 8.800 (1.6); 8.359 (0.9); 8.354 (1.3); 8.349 (0.9); 8.339
(1.0); 8.334 (1.4); 8.329 (0.9); 7.613 (1.2); 7.601 (1.2); 7.593 (1.2); 7.581 (1.1); 4.022 (15.8); 3.329 (3.8); 3.214 (16.0); 3.175
(0.4); 3.164 (0.3); 2.512 (6.0); 2.508 (11.4); 2.503 (14.4); 2.499 (10.3); 2.494 (4.9); 0.000 (5.6)
Compound No. I-1-142: ¹H NMR (400.0 MHz, d₆-DMSO):
11.147 (3.1); 9.112 (2.8); 9.107 (2.9); 8.735 (1.9); 8.731 (2.1); 8.723 (2.1); 8.719 (2.0); 8.326 (1.6); 8.322 (1.1); 8.311 (1.2); 8.306
(1.7); 8.302 (1.1); 7.536 (1.6); 7.524 (1.6); 7.516 (1.6); 7.504 (1.5); 6.562 (4.4); 5.589 (9.9); 3.958 (2.2); 3.939 (4.4); 3.920
(2.6); 3.510 (2.9); 3.491 (4.9); 3.472 (2.4); 3.331 (12.3); 3.176 (0.6); 3.163 (0.6); 2.507 (18.0); 2.503 (23.2); 2.499 (17.4); 2.358
(16.0); 2.335 (0.4); 0.000 (2.3)
Compound No. I-1-143: ¹H NMR (400.0 MHz, d₆-DMSO):
11.125 (2.5); 9.104 (2.2); 9.100 (2.2); 8.722 (1.5); 8.718 (1.7); 8.710 (1.7); 8.706 (1.7); 8.326 (0.9); 8.321 (1.3); 8.316 (1.0); 8.306
(0.9); 8.301 (1.3); 8.296 (0.9); 7.522 (1.2); 7.510 (1.2); 7.503 (1.1); 7.502 (1.1); 7.490 (1.1); 6.499 (3.3); 5.787 (7.7); 3.329
(33.7); 3.105 (16.0); 2.511 (9.5); 2.507 (18.5); 2.503 (24.2); 2.498 (18.2); 2.494 (9.3); 2.443 (11.5); 2.138 (15.8); 1.235 (0.5); 0.008
(0.5); 0.000 (11.0); −0.008 (0.5)
Compound No. I-1-144: ¹H NMR (400.0 MHz, d₆-DMSO):
11.163 (2.2); 9.111 (1.9); 9.107 (1.9); 8.725 (1.6); 8.721 (1.7); 8.713 (1.6); 8.709 (1.6); 8.332 (0.8); 8.328 (1.1); 8.323 (0.8); 8.312
(0.9); 8.307 (1.2); 8.303 (0.8); 7.527 (1.1); 7.525 (1.1); 7.515 (1.1); 7.513 (1.1); 7.507 (1.1); 7.505 (1.0); 7.495 (1.0); 7.493
(1.0); 6.516 (2.9); 5.716 (6.5); 5.460 (0.3); 3.873 (16.0); 3.864 (1.0); 3.327 (18.8); 2.713 (0.6); 2.706 (0.7); 2.697 (1.3); 2.689 (0.6);
2.686 (0.7); 2.681 (0.6); 2.671 (0.4); 2.525 (0.3); 2.512 (7.4); 2.508 (15.0); 2.503 (19.8); 2.498 (14.2); 2.494 (6.7); 2.435 (10.1);
2.261 (0.5); 1.235 (0.4); 0.870 (1.4); 0.864 (2.2); 0.860 (1.6); 0.853 (2.9); 0.848 (4.4); 0.841 (3.5); 0.834 (1.5); 0.008 (0.4);
0.000 (11.5); −0.009 (0.4)
Compound No. I-1-145: ¹H NMR (400.0 MHz, d₆-DMSO):
11.050 (2.1); 9.091 (1.9); 9.087 (1.9); 8.729 (1.5); 8.725 (1.6); 8.717 (1.6); 8.713 (1.6); 8.314 (0.8); 8.309 (1.1); 8.304 (0.8); 8.294
(0.8); 8.289 (1.2); 8.284 (0.8); 7.530 (1.1); 7.528 (1.1); 7.516 (1.1); 7.510 (1.1); 7.509 (1.1); 7.498 (1.0); 7.496 (1.0); 6.529
(3.0); 5.731 (7.2); 3.327 (22.2); 3.176 (1.0); 3.163 (1.0); 3.117 (16.0); 2.919 (16.0); 2.525 (0.4); 2.511 (9.1); 2.507 (18.3); 2.503
(24.2); 2.498 (17.5); 2.494 (8.4); 2.376 (11.1); 0.008 (0.4); 0.000 (11.9); −0.009 (0.4)
Compound No. I-1-146: ¹H NMR (400.0 MHz, d₆-DMSO):
11.132 (2.4); 9.104 (2.1); 9.100 (2.0); 8.723 (1.6); 8.719 (1.7); 8.711 (1.7); 8.707 (1.7); 8.326 (0.9); 8.322 (1.2); 8.317 (0.9); 8.306
(0.9); 8.301 (1.3); 8.297 (0.9); 7.525 (1.1); 7.523 (1.2); 7.513 (1.1); 7.511 (1.2); 7.505 (1.1); 7.503 (1.1); 7.493 (1.1); 7.491
(1.1); 6.497 (3.2); 5.739 (7.2); 3.327 (17.1); 2.801 (0.6); 2.798 (0.6); 2.796 (0.6); 2.793 (0.6); 2.786 (1.3); 2.779 (0.6); 2.774 (0.7);
2.771 (0.5); 2.759 (0.3); 2.525 (0.4); 2.512 (8.2); 2.507 (16.5); 2.503 (21.7); 2.498 (15.6); 2.493 (7.4); 2.431 (10.8); 2.190 (16.0);
2.171 (0.4); 1.235 (0.5); 0.914 (1.5); 0.909 (2.6); 0.899 (3.4); 0.891 (6.0); 0.008 (0.5); 0.000 (12.5); −0.009 (0.4)
Compound No. I-1-147: ¹H NMR (400.0 MHz, d₆-DMSO):
11.150 (3.6); 9.104 (3.1); 9.100 (3.1); 8.723 (2.5); 8.719 (2.7); 8.711 (2.7); 8.707 (2.7); 8.326 (1.3); 8.321 (1.8); 8.316 (1.4); 8.306
(1.4); 8.301 (1.9); 8.296 (1.3); 7.524 (1.8); 7.523 (1.8); 7.512 (1.7); 7.511 (1.7); 7.505 (1.7); 7.503 (1.7); 7.493 (1.7); 7.491
(1.7); 7.250 (0.7); 7.232 (0.7); 7.182 (0.8); 7.164 (0.7); 7.162 (0.6); 7.144 (0.3); 6.498 (4.7); 5.757 (3.9); 5.711 (10.3); 4.056 (1.0);
4.038 (3.0); 4.020 (3.0); 4.002 (1.0); 3.327 (57.5); 3.268 (0.5); 2.897 (0.4); 2.891 (0.6); 2.886 (0.9); 2.878 (1.1); 2.870 (1.9); 2.859
(1.0); 2.853 (0.9); 2.843 (0.5); 2.525 (0.6); 2.520 (1.0); 2.512 (16.4); 2.507 (33.8); 2.503 (44.9); 2.498 (32.2); 2.493 (15.4);
2.440 (0.6); 2.425 (16.0); 2.300 (3.5); 1.989 (13.4); 1.981 (0.6); 1.968 (1.0); 1.960 (1.0); 1.956 (0.7); 1.948 (2.0); 1.939 (0.6);
1.935 (1.1); 1.927 (1.0); 1.914 (0.5); 1.193 (3.7); 1.175 (7.2); 1.157 (3.6); 0.969 (2.3); 0.963 (3.6); 0.959 (2.6); 0.944 (8.4); 0.935
(5.9); 0.930 (4.7); 0.924 (3.6); 0.915 (1.5); 0.906 (0.4); 0.898 (0.4); 0.816 (1.3); 0.806 (3.2); 0.800 (3.0); 0.794 (3.2); 0.788 (3.0);
0.777 (0.9); 0.008 (0.8); 0.000 (25.7); −0.009 (0.8)
Compound No. I-1-148: ¹H NMR (600.1 MHz, d₆-DMSO):
3.344 (16.0); 3.249 (0.5); 2.509 (0.6); 2.506 (1.3); 2.503 (1.8); 2.500 (1.3); 2.497 (0.6); 2.468 (0.4); 2.409 (0.4)
Compound No. I-1-149: ¹H NMR(601.6 MHz, d₆-DMSO):
11.058 (3.2); 9.111 (2.8); 9.108 (2.9); 8.725 (2.1); 8.722 (2.4); 8.717 (2.3); 8.714 (2.5); 8.324 (1.6); 8.310 (1.7); 7.525 (1.5); 7.517
(1.6); 7.511 (1.6); 7.503 (1.5); 6.543 (2.3); 6.484 (4.2); 5.435 (9.8); 5.185 (0.5); 3.344 (15.9); 3.330 (3.7); 3.320 (2.3); 3.092
(1.8); 3.084 (2.7); 2.507 (27.2); 2.504 (38.4); 2.501 (30.7); 2.355 (16.0); 2.183 (0.7); 1.990 (0.4); 1.791 (0.6); 1.782 (1.7); 1.772
(2.5); 1.763 (1.7); 1.753 (0.7); 1.056 (0.4); 0.005 (1.2); 0.000 (28.8)
Compound No. I-1-150: ¹H NMR (400.0 MHz, d₆-DMSO):
11.514 (4.1); 9.633 (2.4); 9.630 (3.1); 9.628 (3.1); 9.625 (2.7); 9.448 (2.5); 9.445 (2.6); 9.435 (2.6); 9.432 (2.6); 8.136 (2.2); 8.130
(2.3); 8.123 (2.2); 8.117 (2.2); 6.512 (5.1); 5.724 (9.8); 3.341 (158.3); 3.250 (0.5); 2.897 (0.4); 2.886 (0.9); 2.879 (1.2); 2.870
(1.8); 2.861 (1.2); 2.854 (1.0); 2.844 (0.5); 2.543 (13.7); 2.526 (0.9); 2.508 (33.5); 2.503 (44.9); 2.499 (34.9); 2.431 (16.0); 1.981
(0.4); 1.969 (0.9); 1.961 (1.0); 1.948 (1.9); 1.936 (1.1); 1.928 (1.0); 1.915 (0.5); 0.991 (0.4); 0.963 (3.8); 0.959 (2.9); 0.952
(5.2); 0.946 (8.1); 0.935 (6.5); 0.932 (7.2); 0.925 (5.4); 0.916 (1.9); 0.811 (1.2); 0.802 (3.1); 0.796 (3.3); 0.790 (3.4); 0.784 (3.2);
0.773 (1.0); 0.000 (0.9)
Compound No. I-1-151: ¹H NMR (400.0 MHz, d₆-DMSO):
11.498 (3.3); 9.631 (2.7); 9.629 (2.6); 9.449 (2.1); 9.447 (2.0); 9.436 (2.2); 9.434 (2.1); 8.138 (1.7); 8.132 (1.8); 8.124 (1.8); 8.119
(1.7); 6.513 (4.2); 5.752 (8.1); 3.348 (65.8); 3.095 (0.5); 2.813 (0.3); 2.801 (0.8); 2.795 (0.9); 2.787 (1.5); 2.777 (0.9); 2.771
(0.8); 2.760 (0.4); 2.544 (51.7); 2.508 (13.9); 2.504 (17.8); 2.500 (14.0); 2.439 (13.0); 2.190 (16.0); 0.910 (3.1); 0.892 (6.5); 0.000
(0.5)
Compound No. I-1-152: ¹H NMR (400.0 MHz, d₆-DMSO):
11.527 (2.8); 9.636 (2.2); 9.633 (2.3); 9.630 (1.9); 9.450 (1.7); 9.448 (1.9); 9.437 (1.8); 9.434 (1.9); 8.141 (1.6); 8.136 (1.6); 8.128
(1.6); 8.122 (1.6); 6.529 (3.7); 5.727 (7.0); 3.870 (16.0); 3.341 (171.2); 2.723 (0.3); 2.713 (0.8); 2.705 (0.9); 2.697 (1.4); 2.687
(0.8); 2.680 (0.8); 2.670 (0.6); 2.542 (29.8); 2.507 (34.9); 2.503 (45.9); 2.499 (35.3); 2.441 (11.7); 2.330 (0.4); 0.864 (2.5); 0.860
(1.9); 0.846 (5.1); 0.839 (4.0); 0.819 (0.4); 0.000 (1.7)
Compound No. I-1-153: ¹H NMR (400.0 MHz, d₆-DMSO):
11.024 (2.2); 9.091 (2.0); 9.087 (2.0); 8.722 (1.5); 8.718 (1.6); 8.710 (1.6); 8.706 (1.5); 8.311 (0.8); 8.307 (1.2); 8.302 (0.8); 8.291
(0.9); 8.287 (1.2); 8.282 (0.8); 7.521 (1.2); 7.509 (1.2); 7.501 (1.2); 7.489 (1.1); 6.540 (3.2); 5.215 (8.2); 4.992 (0.4); 3.329
(29.9); 3.307 (16.5); 3.284 (1.5); 3.263 (0.9); 3.187 (1.1); 3.129 (1.0); 3.084 (1.1); 3.069 (16.0); 2.965 (1.0); 2.507 (20.8); 2.503
(26.9); 2.498 (19.7); 2.350 (12.1); 2.180 (0.7); 2.153 (0.8); 1.235 (0.8); 0.000 (0.5)
Compound No. I-1-154: ¹H NMR (400.0 MHz, d₆-DMSO):
11.077 (3.2); 9.107 (3.0); 9.102 (3.0); 8.724 (2.0); 8.720 (2.2); 8.712 (2.2); 8.708 (2.1); 8.321 (1.8); 8.317 (1.4); 8.306 (1.3); 8.301
(1.8); 7.525 (1.6); 7.513 (1.7); 7.505 (1.7); 7.493 (1.5); 6.561 (4.5); 6.087 (5.5); 5.756 (1.9); 5.087 (8.8); 3.326 (9.2); 2.507
(30.1); 2.503 (38.0); 2.499 (29.8); 2.336 (16.0); 2.118 (14.2); 0.000 (38.9)
Compound No. I-1-155: ¹H NMR (400.0 MHz, d₆-DMSO):
11.036 (3.3); 9.200 (2.9); 9.099 (2.9); 9.094 (2.9); 8.718 (2.1); 8.714 (2.3); 8.706 (2.2); 8.702 (2.2); 8.317 (1.1); 8.313 (1.9); 8.307
(1.2); 8.297 (1.2); 8.292 (1.7); 8.287 (1.2); 7.850 (5.3); 7.721 (2.0); 7.697 (2.5); 7.521 (1.6); 7.509 (1.6); 7.501 (1.6); 7.489
(1.6); 7.475 (2.1); 7.471 (2.2); 7.452 (1.8); 7.447 (1.8); 6.525 (4.4); 5.362 (8.3); 3.913 (0.5); 3.446 (0.4); 3.351 (732.8); 3.249 (0.7);
3.170 (6.1); 3.099 (0.5); 2.996 (0.7); 2.677 (0.7); 2.673 (0.9); 2.668 (0.7); 2.543 (19.5); 2.526 (2.2); 2.513 (51.4); 2.508 (104.6);
2.504 (139.9); 2.499 (105.0); 2.495 (54.0); 2.376 (16.0); 2.335 (0.9); 2.331 (1.1); 2.326 (0.8); 2.235 (0.3); 2.141 (0.3); 0.008
(2.6); 0.000 (72.2); −0.008 (3.3)
Compound No. I-1-156: ¹H NMR (400.0 MHz, d₆-DMSO):
11.066 (3.3); 9.111 (3.1); 9.107 (3.2); 8.722 (2.4); 8.718 (2.4); 8.710 (2.6); 8.706 (2.3); 8.531 (2.2); 8.527 (2.3); 8.519 (2.4); 8.516
(2.3); 8.331 (1.1); 8.327 (1.7); 8.322 (1.3); 8.316 (0.7); 8.312 (1.3); 8.307 (1.8); 8.302 (1.3); 8.208 (2.0); 8.205 (1.9); 8.188
(2.2); 8.185 (2.0); 7.648 (2.1); 7.636 (2.1); 7.628 (2.0); 7.616 (1.9); 7.525 (1.7); 7.513 (1.8); 7.505 (1.7); 7.493 (1.7); 6.734 (2.6);
6.703 (0.3); 6.522 (4.3); 6.408 (0.4); 5.323 (0.4); 5.289 (8.0); 5.269 (0.5); 3.334 (11.8); 2.996 (0.4); 2.712 (0.5); 2.676 (0.4); 2.672
(0.6); 2.667 (0.4); 2.542 (129.7); 2.525 (1.6); 2.507 (61.5); 2.502 (79.6); 2.498 (58.0); 2.494 (28.5); 2.368 (0.7); 2.333 (16.0);
2.235 (2.1); 2.170 (0.7); 1.121 (1.0); 1.106 (1.0); 0.008 (1.6); 0.000 (44.1); −0.009 (1.7)
Compound No. I-1-157: ¹H NMR (400.0 MHz, d₆-DMSO):
11.064 (3.3); 10.367 (0.4); 9.112 (2.8); 9.107 (2.9); 8.982 (0.4); 8.977 (0.4); 8.727 (1.9); 8.724 (2.1); 8.716 (2.1); 8.712 (2.1); 8.329
(1.1); 8.324 (1.6); 8.320 (1.2); 8.309 (1.2); 8.304 (1.7); 8.300 (1.2); 7.531 (1.6); 7.519 (1.6); 7.511 (1.6); 7.499 (1.5); 6.914
(0.9); 6.910 (1.0); 6.906 (0.9); 6.900 (1.6); 6.893 (1.4); 6.889 (1.9); 6.858 (2.5); 6.852 (12.8); 6.845 (4.8); 6.838 (2.4); 6.827 (1.0);
6.820 (0.4); 6.799 (0.4); 6.707 (0.8); 6.697 (0.4); 6.501 (4.4); 6.146 (0.5); 4.609 (0.3); 4.604 (0.4); 4.594 (1.1); 4.589 (1.2); 4.579
(1.4); 4.574 (1.3); 4.565 (0.7); 4.559 (0.6); 4.360 (1.8); 4.354 (1.7); 4.343 (0.5); 4.331 (2.2); 4.325 (2.0); 4.306 (5.1); 4.291
(4.2); 4.272 (0.3); 4.068 (1.5); 4.053 (1.5); 4.039 (1.4); 4.024 (1.3); 4.016 (0.4); 3.326 (17.1); 2.507 (34.3); 2.502 (45.4); 2.498
(35.3); 2.329 (0.3); 2.253 (16.0); 2.175 (2.3); 1.235 (1.3); 0.007 (1.3); 0.000 (29.0)
Compound No. I-1-158: ¹H NMR (400.0 MHz, d₆-DMSO):
11.026 (3.0); 9.106 (2.4); 9.103 (2.6); 8.719 (2.0); 8.715 (2.3); 8.707 (2.2); 8.703 (2.2); 8.325 (0.9); 8.320 (1.4); 8.316 (1.4); 8.306
(1.0); 8.300 (1.5); 8.296 (1.1); 7.520 (1.7); 7.508 (1.6); 7.500 (1.6); 7.488 (1.6); 7.027 (0.5); 7.015 (1.7); 6.996 (2.3); 6.979
(1.1); 6.961 (0.7); 6.953 (0.4); 6.940 (0.6); 6.936 (0.6); 6.919 (0.3); 6.860 (4.7); 6.841 (1.5); 6.566 (1.1); 6.512 (3.1); 6.347 (0.4);
6.329 (0.4); 6.104 (0.3); 5.756 (16.0); 5.170 (2.2); 5.130 (5.8); 5.118 (0.9); 3.324 (35.3); 2.890 (0.4); 2.753 (0.6); 2.738 (1.1);
2.732 (1.1); 2.722 (0.8); 2.670 (5.3); 2.667 (5.2); 2.606 (0.6); 2.595 (0.6); 2.524 (1.2); 2.519 (1.7); 2.511 (27.5); 2.506 (58.4); 2.502
(80.3); 2.497 (61.0); 2.493 (31.8); 2.333 (0.5); 2.329 (0.7); 2.324 (0.5); 2.255 (12.0); 2.216 (4.4); 2.195 (0.4); 2.191 (0.4); 2.159
(1.1); 2.142 (1.5); 2.093 (0.4); 2.059 (0.4); 2.046 (0.6); 1.803 (0.4); 1.791 (0.6); 1.776 (0.7); 1.760 (0.5); 1.719 (3.0); 1.703
(6.7); 1.695 (4.5); 1.687 (3.1); 1.675 (1.9); 1.234 (1.6); 0.008 (1.7); 0.000 (61.8); −0.008 (3.1)
Compound No. I-1-159: ¹H NMR (400.0 MHz, d₆-DMSO):
11.024 (0.6); 9.102 (0.5); 9.098 (0.6); 8.718 (0.7); 8.713 (0.7); 8.706 (0.5); 8.702 (0.4); 8.294 (0.3); 7.711 (1.0); 6.514 (0.8); 5.310
(1.6); 4.121 (1.3); 4.107 (3.8); 4.094 (3.9); 4.081 (1.4); 3.339 (9.5); 3.178 (16.0); 3.165 (15.6); 2.508 (4.9); 2.504 (6.5); 2.500
(5.1); 2.374 (2.9); 0.000 (4.6)
Compound No. I-1-160: ¹H NMR (400.0 MHz, d₆-DMSO):
11.028 (3.5); 9.103 (3.1); 9.098 (3.1); 8.794 (3.6); 8.791 (3.6); 8.718 (2.0); 8.714 (2.2); 8.706 (2.2); 8.702 (2.2); 8.315 (1.7); 8.295
(1.8); 7.699 (5.5); 7.568 (2.7); 7.544 (3.2); 7.519 (1.6); 7.507 (1.7); 7.500 (1.6); 7.488 (1.5); 7.289 (2.3); 7.284 (2.4); 7.265
(2.0); 7.260 (2.0); 6.517 (4.7); 5.318 (9.0); 3.329 (22.4); 2.508 (22.1); 2.504 (28.5); 2.500 (22.5); 2.371 (16.0); 0.000 (33.8)
Compound No. I-1-161: ¹H NMR (400.0 MHz, d₆-DMSO):
11.126 (2.9); 9.119 (2.8); 9.114 (2.9); 8.757 (2.1); 8.753 (2.3); 8.745 (2.2); 8.741 (2.3); 8.337 (1.1); 8.332 (1.7); 8.328 (1.2); 8.317
(1.3); 8.312 (1.8); 8.308 (1.2); 8.134 (0.4); 7.559 (1.6); 7.547 (1.6); 7.539 (1.6); 7.527 (1.5); 6.665 (4.1); 5.756 (7.0); 3.670
(1.2); 3.609 (0.4); 3.592 (0.4); 3.575 (0.3); 3.492 (1.4); 3.355 (1.0); 3.239 (0.6); 3.014 (0.9); 2.525 (0.5); 2.507 (27.5); 2.503 (36.7);
2.498 (27.7); 2.421 (16.0); 1.861 (5.8); 1.739 (0.6); 1.253 (0.6); 1.235 (0.4); 1.180 (0.6); 0.008 (0.7); 0.000 (21.4); −0.008
(1.0)
Compound No. I-1-162: ¹H NMR (600.1 MHz, d₆-DMSO):
11.412 (3.3); 9.135 (2.9); 9.134 (3.0); 9.131 (3.0); 8.760 (2.4); 8.757 (2.5); 8.752 (2.5); 8.749 (2.5); 8.351 (1.3); 8.349 (1.8); 8.345
(1.2); 8.338 (1.3); 8.335 (1.8); 8.332 (1.3); 7.555 (1.7); 7.554 (1.6); 7.547 (1.7); 7.546 (1.6); 7.542 (1.7); 7.540 (1.6); 7.534
(1.6); 7.532 (1.5); 6.815 (4.3); 3.769 (1.8); 3.749 (1.9); 3.327 (25.2); 2.835 (1.1); 2.831 (1.3); 2.814 (2.4); 2.810 (2.4); 2.793 (1.3);
2.789 (1.1); 2.511 (4.7); 2.508 (10.2); 2.505 (14.0); 2.502 (10.1); 2.499 (4.7); 2.478 (16.0); 2.477 (15.3); 1.990 (0.5); 1.678 (1.5);
1.674 (1.5); 1.656 (1.6); 1.653 (1.6); 1.456 (0.5); 1.450 (0.6); 1.445 (0.5); 1.439 (0.6); 1.432 (0.5); 1.426 (0.4); 1.420 (0.3);
1.134 (0.6); 1.127 (0.6); 1.113 (1.3); 1.107 (1.3); 1.093 (1.2); 1.086 (1.2); 1.072 (0.5); 1.065 (0.5); 0.870 (8.7); 0.859 (8.5)
Compound No. I-1-163: ¹H NMR(601.6 MHz, d₆-DMSO):
19.977 (0.5); 11.275 (12.2); 9.126 (12.4); 8.762 (8.4); 8.754 (8.6); 8.336 (6.4); 8.323 (7.0); 7.698 (3.6); 7.689 (6.9); 7.680 (3.9);
7.564 (5.5); 7.556 (5.9); 7.551 (6.1); 7.543 (5.5); 6.735 (16.0); 5.974 (0.5); 5.762 (1.0); 3.484 (5.6); 3.475 (15.7); 3.467 (13.6);
3.447 (7.4); 3.439 (12.7); 3.429 (10.5); 3.393 (0.6); 3.348 (84.9); 3.296 (1.1); 3.279 (70.5); 3.237 (5.8); 3.159 (0.4); 3.147 (0.6);
3.137 (1.3); 3.129 (1.2); 2.617 (0.5); 2.537 (54.5); 2.505 (90.0); 2.427 (0.4); 2.390 (0.6); 0.000 (44.0)
Compound No. I-1-164: ¹H NMR (400.0 MHz, d₆-DMSO):
9.122 (0.9); 9.118 (0.9); 8.766 (0.6); 8.762 (0.6); 8.754 (0.6); 8.750 (0.6); 8.337 (0.3); 8.332 (0.5); 8.327 (0.4); 8.317 (0.5); 8.312
(0.6); 8.307 (0.4); 7.783 (0.4); 7.569 (0.5); 7.557 (0.5); 7.550 (0.5); 7.538 (0.4); 6.732 (1.3); 4.252 (0.3); 4.237 (0.5); 4.223 (0.4);
4.028 (0.6); 4.012 (0.6); 4.006 (0.8); 3.990 (1.4); 3.974 (1.8); 3.960 (1.7); 3.947 (3.0); 3.928 (2.7); 3.912 (1.6); 3.723 (0.5); 3.709
(0.6); 3.702 (0.5); 3.687 (0.5); 3.634 (1.8); 3.619 (2.2); 3.615 (2.1); 3.600 (1.4); 3.457 (0.4); 3.438 (0.4); 3.424 (0.6); 3.409
(0.4); 3.364 (0.5); 3.349 (0.7); 3.334 (0.6); 3.315 (0.5); 3.300 (0.4); 3.277 (0.3); 3.261 (0.3); 3.160 (0.3); 2.648 (0.7); 2.634 (0.7);
2.616 (2.5); 2.602 (2.4); 2.590 (2.4); 2.576 (2.1); 2.558 (0.9); 2.537 (4.3); 2.507 (10.2); 2.503 (13.2); 2.499 (10.2); 1.362 (4.3);
1.328 (1.4); 1.305 (14.7); 1.270 (4.5); 1.250 (16.0); 1.112 (0.4); 0.008 (0.7); 0.000 (10.7)
Compound No. I-1-165: ¹H NMR(601.6 MHz, d₆-DMSO):
9.121 (1.4); 9.118 (1.4); 8.761 (1.0); 8.759 (1.0); 8.753 (1.0); 8.751 (1.0); 8.330 (0.5); 8.327 (0.8); 8.321 (2.9); 8.317 (0.7); 8.313
(0.9); 8.310 (0.6); 7.924 (0.3); 7.914 (0.7); 7.905 (0.4); 7.563 (0.7); 7.555 (0.7); 7.550 (0.7); 7.542 (0.7); 6.705 (1.9); 3.347 (1.0);
3.336 (1.8); 3.325 (1.7); 3.314 (0.9); 3.041 (0.8); 3.031 (0.9); 3.020 (0.4); 2.590 (1.6); 2.579 (3.2); 2.568 (1.7); 2.529 (6.8); 2.507
(14.6); 2.504 (20.6); 2.501 (16.6); 2.490 (2.0); 2.485 (2.9); 2.472 (2.1); 2.444 (0.9); 2.432 (1.2); 2.420 (0.9); 2.235 (0.6); 2.054
(11.8); 2.028 (8.8); 2.026 (16.0); 1.810 (1.0); 1.798 (1.5); 1.787 (1.0); 1.621 (0.6); 1.609 (0.8); 1.601 (0.7); 1.597 (0.7); 1.590
(1.5); 1.578 (1.9); 1.566 (1.5); 1.555 (0.5); 0.000 (13.3)
Compound No. I-1-166: ¹H NMR (400.0 MHz, d₆-DMSO):
9.136 (4.0); 9.130 (4.0); 8.764 (2.8); 8.760 (3.1); 8.752 (3.0); 8.748 (3.1); 8.350 (1.5); 8.345 (2.4); 8.340 (1.7); 8.330 (1.7); 8.325
(2.6); 8.320 (1.8); 7.564 (2.2); 7.552 (2.2); 7.544 (2.3); 7.532 (4.1); 7.510 (2.2); 6.741 (5.5); 6.740 (5.9); 4.058 (0.4); 4.045 (0.9);
4.042 (0.8); 4.028 (1.1); 4.024 (1.1); 4.007 (0.9); 3.993 (0.5); 3.464 (0.5); 3.440 (1.2); 3.426 (1.2); 3.416 (3.7); 3.402 (6.2); 3.389
(3.7); 3.377 (1.5); 3.364 (1.6); 3.335 (3.2); 3.290 (34.1); 3.280 (2.1); 3.273 (0.6); 3.261 (0.5); 3.243 (3.5); 3.236 (16.0); 3.116
(0.8); 3.112 (0.4); 3.103 (0.8); 3.094 (1.7); 3.080 (1.9); 3.068 (1.6); 3.051 (1.9); 3.046 (0.7); 3.029 (0.8); 2.938 (0.6); 2.921
(1.0); 2.907 (0.8); 2.891 (0.5); 2.673 (0.4); 2.538 (20.8); 2.522 (0.7); 2.513 (9.3); 2.509 (19.6); 2.504 (26.6); 2.500 (19.6); 2.495
(9.7); 2.483 (0.4); 2.465 (0.3); 2.236 (0.4); 2.098 (0.4); 2.025 (1.2); 1.187 (12.4); 1.170 (12.2); 1.003 (1.5); 0.987 (1.4); 0.918
(7.2); 0.911 (0.5); 0.902 (7.1); 0.000 (2.1)
Compound No. I-1-167: ¹H NMR (400.0 MHz, d₆-DMSO):
9.123 (2.3); 9.118 (2.5); 8.765 (1.4); 8.762 (1.7); 8.753 (1.6); 8.750 (1.7); 8.337 (0.9); 8.332 (1.4); 8.328 (1.0); 8.317 (1.3); 8.313
(1.5); 8.308 (1.1); 7.941 (0.7); 7.926 (1.3); 7.911 (0.7); 7.568 (1.2); 7.556 (1.3); 7.548 (1.3); 7.536 (1.2); 6.718 (3.5); 3.484 (1.2);
3.467 (2.8); 3.451 (2.9); 3.435 (1.5); 3.339 (1.2); 3.326 (1.2); 3.283 (1.0); 3.232 (0.7); 3.215 (0.8); 3.197 (0.8); 3.188 (0.7); 3.182
(0.6); 3.172 (0.9); 3.155 (0.9); 3.139 (0.5); 3.111 (0.3); 2.688 (2.8); 2.672 (5.7); 2.658 (3.3); 2.537 (11.9); 2.508 (18.5); 2.504
(24.5); 2.500 (20.8); 2.483 (4.3); 2.466 (4.2); 2.450 (2.0); 2.097 (16.0); 2.076 (0.7); 2.068 (0.8); 2.062 (1.0); 2.054 (3.9); 2.049
(5.5); 2.026 (12.9); 1.795 (2.3); 0.000 (1.3)
Compound No. I-1-168: ¹H NMR (400.0 MHz, d₆-DMSO):
11.226 (11.5); 9.119 (9.6); 9.114 (10.0); 8.761 (6.6); 8.757 (7.4); 8.749 (7.2); 8.745 (7.4); 8.332 (3.8); 8.327 (5.7); 8.322 (4.2);
8.316 (1.3); 8.312 (4.2); 8.307 (6.0); 8.302 (4.3); 7.921 (2.8); 7.907 (5.9); 7.892 (3.0); 7.562 (5.0); 7.561 (5.1); 7.549 (5.1); 7.542
(5.1); 7.541 (5.1); 7.530 (4.8); 7.529 (4.8); 7.319 (8.0); 7.314 (3.5); 7.307 (1.9); 7.301 (12.1); 7.298 (13.9); 7.284 (4.2); 7.279
(12.2); 7.274 (5.2); 7.261 (1.1); 7.255 (2.9); 6.976 (16.0); 6.968 (6.1); 6.966 (6.1); 6.956 (13.4); 6.954 (13.1); 6.947 (12.0); 6.929
(5.2); 6.924 (6.6); 6.904 (1.1); 6.734 (14.1); 6.732 (14.3); 6.253 (0.7); 6.239 (1.5); 6.224 (0.8); 5.756 (1.6); 4.148 (7.1); 4.133
(15.9); 4.119 (8.2); 3.958 (2.2); 3.944 (4.9); 3.930 (2.5); 3.675 (4.0); 3.661 (11.0); 3.647 (10.7); 3.633 (3.7); 3.397 (1.2); 3.383
(3.4); 3.369 (3.3); 3.354 (1.2); 3.331 (54.7); 2.677 (0.4); 2.672 (0.5); 2.668 (0.4); 2.550 (46.9); 2.526 (1.3); 2.512 (28.3); 2.508
(58.2); 2.503 (79.0); 2.499 (61.1); 2.335 (0.4); 2.330 (0.5); 2.326 (0.4); 0.008 (0.7); 0.000 (21.2); −0.008 (1.2)
Compound No. I-1-169: ¹H NMR (400.0 MHz, d₆-DMSO):
10.933 (2.0); 9.035 (1.8); 9.031 (1.8); 8.812 (1.1); 8.808 (1.3); 8.800 (1.2); 8.796 (1.3); 8.233 (0.6); 8.228 (1.0); 8.224 (0.7); 8.213
(0.7); 8.208 (1.0); 8.204 (0.7); 7.628 (0.9); 7.616 (0.9); 7.608 (0.9); 7.596 (0.8); 6.424 (3.5); 3.690 (1.7); 3.602 (2.4); 3.589
(3.5); 3.332 (12.5); 3.295 (0.5); 3.270 (0.6); 3.242 (16.0); 2.542 (1.8); 2.507 (13.3); 2.503 (17.2); 2.499 (13.5); 2.208 (9.7); 0.000
(3.8)
Compound No. I-1-170: ¹H NMR(601.6 MHz, d₆-DMSO):
9.121 (1.8); 9.118 (1.8); 8.765 (1.4); 8.763 (1.4); 8.757 (1.4); 8.755 (1.4); 8.333 (0.7); 8.330 (1.0); 8.327 (0.7); 8.320 (0.9); 8.317
(1.1); 8.314 (0.7); 7.630 (0.5); 7.621 (0.7); 7.566 (1.0); 7.558 (1.0); 7.553 (1.0); 7.545 (0.9); 6.741 (2.7); 4.660 (0.9); 4.652 (2.0);
4.643 (0.9); 4.487 (0.4); 4.385 (0.6); 4.336 (1.0); 4.327 (2.2); 4.318 (1.1); 3.671 (0.5); 3.659 (1.6); 3.655 (0.7); 3.647 (1.6); 3.643
(2.0); 3.636 (0.6); 3.631 (2.0); 3.620 (0.7); 3.617 (0.7); 3.610 (0.4); 3.605 (1.8); 3.601 (1.2); 3.594 (2.0); 3.589 (2.6); 3.585
(0.8); 3.582 (1.0); 3.578 (2.3); 3.574 (0.7); 3.566 (0.7); 3.532 (0.6); 3.520 (1.9); 3.516 (0.6); 3.508 (2.0); 3.504 (1.6); 3.497 (0.7);
3.492 (1.6); 3.481 (0.7); 3.478 (0.9); 3.466 (2.5); 3.462 (0.8); 3.454 (2.7); 3.450 (2.1); 3.442 (1.1); 3.438 (2.0); 3.427 (0.7); 3.376
(2.5); 3.366 (4.7); 3.357 (5.3); 3.075 (0.5); 3.066 (0.8); 3.056 (0.4); 2.588 (0.7); 2.579 (0.7); 2.538 (11.6); 2.529 (3.0); 2.510
(7.7); 2.507 (16.5); 2.505 (22.6); 2.502 (16.4); 1.141 (6.7); 1.129 (14.3); 1.125 (8.4); 1.118 (9.0); 1.113 (16.0); 1.107 (3.7); 1.104
(3.1); 1.101 (7.8); 1.095 (1.7); 0.005 (0.5); 0.000 (16.2); −0.006 (0.6)
Compound No. I-1-171: ¹H NMR (400.0 MHz, d₆-DMSO):
11.240 (4.2); 9.124 (3.5); 9.119 (3.6); 8.764 (2.4); 8.760 (2.7); 8.752 (2.6); 8.748 (2.7); 8.339 (1.3); 8.334 (2.0); 8.329 (1.5); 8.319
(1.5); 8.314 (2.2); 8.309 (1.6); 7.942 (1.0); 7.928 (2.1); 7.913 (1.1); 7.566 (1.9); 7.554 (1.9); 7.546 (1.9); 7.534 (1.8); 7.177
(0.4); 7.157 (5.9); 7.152 (3.3); 7.142 (5.4); 7.132 (6.2); 7.123 (2.3); 7.118 (2.6); 7.106 (0.7); 6.738 (5.0); 6.736 (5.2); 6.284 (0.4);
6.270 (0.7); 6.255 (0.4); 5.758 (16.0); 4.259 (2.4); 4.246 (5.2); 4.232 (2.7); 4.069 (1.0); 4.055 (2.2); 4.041 (1.1); 3.641 (1.5); 3.627
(3.9); 3.613 (3.8); 3.599 (1.3); 3.358 (0.7); 3.343 (2.4); 3.334 (39.9); 3.315 (0.8); 2.542 (17.3); 2.527 (0.8); 2.513 (11.3); 2.509
(23.2); 2.504 (31.1); 2.500 (23.7); 0.008 (0.3); 0.000 (9.5)
Compound No. I-1-172: ¹H NMR (400.0 MHz, d₆-DMSO):
11.207 (10.8); 9.121 (9.1); 9.117 (9.2); 8.767 (6.5); 8.763 (7.1); 8.755 (7.0); 8.751 (7.1); 8.336 (3.7); 8.331 (5.4); 8.326 (3.9); 8.316
(4.2); 8.311 (5.7); 8.306 (4.0); 8.041 (2.5); 8.026 (5.2); 8.012 (2.6); 7.571 (4.8); 7.570 (4.9); 7.559 (4.8); 7.558 (4.9); 7.551
(4.8); 7.550 (4.8); 7.539 (4.5); 7.538 (4.6); 6.727 (13.2); 4.241 (7.8); 4.227 (16.0); 4.214 (8.6); 3.613 (3.5); 3.599 (9.2); 3.585
(8.9); 3.572 (3.3); 3.330 (64.6); 2.677 (0.4); 2.672 (0.6); 2.668 (0.4); 2.540 (45.1); 2.526 (2.4); 2.521 (2.5); 2.512 (28.8); 2.508
(59.2); 2.503 (79.8); 2.499 (60.0); 2.495 (30.9); 2.335 (0.4); 2.330 (0.5); 2.326 (0.4); 1.769 (0.8); 1.234 (1.0); 1.173 (0.5); 1.152
(0.4); 1.133 (0.4); 0.991 (0.5); 0.146 (0.4); 0.008 (3.4); 0.000 (95.6); −0.008 (4.7); −0.150 (0.4)
Compound No. I-1-173: ¹H NMR (DMSO-d6) 2.26 (s, 3H), 3.22 (s, 3H), 3.62-3.65 (t, 2H), 4.10-4.12 (t, 2H),
6.43 (s, 1H), 7.49-7.53 (m, 1H), 8.29-8.32 (m, 1H), 8.71-8.72 (m, 1H), 9.10-9.11 (m, 1H), 11.00 (s, 1H).
Compound No. I-1-174: ¹H NMR (DMSO-d6) 0.32-0.35 (m, 2H), 0.48-0.52 (m, 2H), 1.15-1.23 (m, 1H),
2.27 (s, 3H), 3.84-3.86 (d, 2H), 6.45 (s, 1H), 7.49-7.52 (m, 1H), 8.30-8.32 (m, 1H), 8.71-8.72 (m, 1H), 9.10 (s,
1H), 11.00 (s, 1H).
Compound No. I-1-175: ¹H NMR (DMSO-d6) 2.32 (s, 3H), 3.23 (2, 3H), 5.29 (s, 2H), 6.57 (s, 1H),
7.50-7.54 (m, 1H), 8.30-8.33 (m, 1H), 8.72-8.73 (m, 1H), 9.11 (s, 1H), 11.10 (s, 1H).
Compound No. I-1-176: ¹H NMR (DMSO-d6) 1.64-1.70 (m, 1H), 1.75-1.82 (m, 2H), 1.88-1.95 (m, 1H),
2.27 (s, 3H), 3.59-3.64 (m, 1H), 3.70-3.75 (m, 1H), 3.99-4.00 (d, 2H), 4.11-4.16 (m, 1H), 6.44 (s, 1H),
7.49-7.53 (m, 1H), 8.29-8.32 (m, 1H), 8.71-8.72 (m, 1H), 9.11 (s, 1H), 11.00 (s, 1H).
Compound No. I-1-177: ¹H NMR (DMSO-d6) 0.09-0.32 (m, 4H), 0.95 (m, 1H), 2.17 (s, 3H), 3.28 (s, 3H),
3.73-3.75 (d, 2H), 5.87 (s, 1H), 7.32 (m, 1H), 7.68 (m, 1H), 8.46-8.50 (m, 2H).
Compound No. I-1-178: ¹H NMR (DMSO-d6) 2.19 (s, 3H), 2.87 (s, 3H), 3.30 (s, 3H), 5.17 (s, 2H), 6.03 (s, 1H),
7.32-7.35 (m, 1H), 7.72-7.73 (m, 1H), 8.48-8.50 (m, 2H).
Compound No. I-1-179: ¹H NMR (DMSO-d6) 2.15 (s, 3H), 3.09 (s, 3H), 3.21-3.46 (m. 5H). 3.97-3.99 (m,
2H), 5.84 (s, 1H), 7.32 (m, 1H), 7.68 (m, 1H), 8.46-8.51 (m, 2H).
Compound No. I-1-180: ¹H NMR (DMSO-d6) 2.28 (s, 3H), 3.79-3.90 (m, 4H), 4.10-4.11 (d, 2H),
5.13-5.15 (t, 1H), 6.47 (s, 1H), 7.49-7.53 (m, 1H), 8.30-8.33 (m, 1H), 8.71-8.72 (m, 1H), 9.11 (s, 1H), 11.05 (s, 1H).
Compound No. I-1-181: ¹H NMR (DMSO-d6) 1.58-1.78 (m, 4H), 2.16 (s, 3H), 3.28 (s, 3H), 3.54-3.59 (m, 2H),
3.86-3.91 (m, 3H), 5.87 (s, 1H), 7.32 (m, 1H), 7.69 (m, 1H), 8.46-8.51 (m, 2H).
Compound No. I-1-182: ¹H NMR (DMSO-d6) 2.32 (s, 3H), 2.91 (s, 3H), 3.61-3.64 (t, 2H), 4.37-4.40 (t, 2H),
6.48 (s, 1H), 7.50-7.53 (m, 1H), 8.30-8.32 (m, 1H), 8.72-8.73 (m, 1H), 9.10 (s, 1H), 11.06 (s, 1H).
Compound No. I-1-183: ¹H NMR (DMSO-d6) 2.31 (s, 3H), 2.56 (s, 3H), 3.04-3.10 (m, 1H), 3.22-3.29 (m, 1H),
4.33-4.37 (m, 2H), 6.48 (s, 1H), 7.50-7.53 (m, 1H), 8.29-8.32 (m, 1H), 8.71-8.73 (m, 1H), 9.10 (s, 1H),
11.05 (s, 1H).
Compound No. I-1-184: ¹H NMR (DMSO-d6) 2.03 (s, 3H), 2.31 (s, 3H), 2.83-2.87 (t, 2H), 4.13-4.17 (t, 2H),
6.46 (s, 1H), 7.49-7.53 (m, 1H), 8.30-8.32 (m, 1H), 8.71-8.72 (m, 1H), 9.11 (s, 1H), 11.03 (s, 1H).
Compound No. I-1-185: ¹H NMR (DMSO-d6) 2.32 (s, 3H), 2.97-3.01 (t, 2H), 4.25-4.28 (t, 2H), 6.50 (s, 1H),
7.50-7.53 (m, 1H), 8.30-8.32 (m, 1H), 8.72-8.73 (m, 1H), 9.11 (s, 1H), 11.07 (s, 1H).
Compound No. I-1-186: ¹H NMR (DMSO-d6) 2.19 (s, 3H), 2.46 (s, 3H), 2.92 (m, 2H), 3.29 (s, 3H),
4.22-4.24 (m, 2H), 5.87 (s, 1H), 7.33 (m, 1H), 7.68 (m, 1H), 8.46-8.53 (m, 2H).
Compound No. I-1-187: ¹H NMR (DMSO-d6) 1.93 (s, 3H), 2.19 (s, 3H), 2.60 (m, 2H), 3.28 (s, 3H),
4.02-4.05 (m, 2H), 5.83 (s, 1H), 7.31 (m, 1H), 7.68 (m, 1H), 8.47-8.51 (m, 2H).
Compound No. I-1-188: ¹H NMR (DMSO-d6) 2.28 (s, 3H), 2.77-2.85 (m, 2H), 4.20-4.24 (t, 2H), 6.47 (s, 1H),
7.50-7.53 (m, 1H), 8.29-8.32 (m, 1H), 8.71-8.72 (m, 1H), 9.10 (s, 1H), 11.05 (s, 1H).
Compound No. I-1-189: ¹H NMR (DMSO-d6) 1.15-1.18 (t, 3H), 2.30 (s, 3H), 2.46-2.52 (m, 2H), 2.86-2.90 (t,
2H), 4.12-4.15 (t, 2H), 6.45 (s, 1H), 7.50-7.53 (m, 1H), 8.30-8.32 (m, 1H), 8.71-8.72 (m, 1H), 9.11 (s, 1H),
11.03 (s, 1H).
Compound No. I-1-190: ¹H NMR (DMSO-d6) 1.16-1.19 (t, 3H), 2.31 (s, 3H), 2.61-2.70 (m, 1H),
2.75-2.82 (m, 1H), 3.02-3.08 (m, 1H), 3.17-3.24 (m, 1H), 4.30-4.40 (m, 2H), 6.48 (s, 1H), 7.50-7.53 (m, 1H),
8.29-8.32 (m. 1H); 8.72-8.73 (m, 1H), 9.10 (s, 1H), 11.05 (s, 1H).
Compound No. I-1-191: ¹H NMR (DMSO-d6) 2.26 (s, 3H), 3.68 (s, 3H), 4.24-4.27 (t, 2H), 4.38-4.40 (t, 2H),
6.47 (s, 1H), 7.50-7.53 (m, 1H), 8.30-8.32 (m, 1H), 8.72 (m, 1H), 9.11 (s, 1H), 11.04 (s, 1H).
Compound No. I-1-192: ¹H NMR (400.0 MHz, d₆-DMSO):
11.078 (2.6); 9.094 (2.4); 9.090 (2.4); 8.723 (1.6); 8.719 (1.7); 8.711 (1.7); 8.707 (1.6); 8.313 (0.9); 8.308 (1.4); 8.304 (0.9); 8.293
(1.0); 8.288 (1.5); 8.284 (1.0); 7.744 (2.8); 7.739 (2.9); 7.522 (1.3); 7.509 (1.3); 7.502 (1.3); 7.490 (1.2); 6.506 (3.6); 6.173
(8.4); 6.066 (2.9); 6.061 (2.9); 5.756 (2.9); 3.327 (15.2); 2.525 (0.6); 2.511 (9.0); 2.507 (17.8); 2.502 (23.3); 2.498 (17.0); 2.464
(13.7); 2.135 (16.0); 0.008 (1.1); 0.000 (28.7); −0.008 (1.2)
Compound No. I-1-193: ¹H NMR (400.0 MHz, d₆-DMSO):
11.221 (3.0); 9.145 (1.4); 8.742 (1.1); 8.733 (1.1); 8.362 (1.0); 8.358 (1.6); 8.353 (1.1); 8.342 (1.1); 8.338 (1.7); 8.333 (1.1); 7.549
(1.3); 7.537 (1.3); 7.529 (1.3); 7.517 (1.2); 7.436 (4.0); 7.415 (6.2); 7.336 (4.9); 7.315 (3.4); 6.732 (4.3); 3.328 (23.9); 3.176
(0.3); 3.163 (0.3); 2.525 (0.6); 2.511 (14.1); 2.507 (27.9); 2.502 (36.1); 2.498 (26.2); 2.494 (12.7); 2.373 (14.1); 2.338 (16.0); 2.320
(0.8); 2.216 (0.6); 2.075 (0.4); 0.008 (1.4); 0.000 (35.4); −0.008 (1.4)
Compound No. I-1-194: ¹H NMR (400.0 MHz, d₆-DMSO):
11.160 (2.7); 9.125 (2.2); 9.121 (2.2); 8.825 (7.1); 8.813 (7.2); 8.779 (1.8); 8.775 (1.8); 8.767 (1.9); 8.763 (1.8); 8.338 (0.9); 8.334
(1.3); 8.328 (0.9); 8.318 (1.0); 8.313 (1.3); 8.308 (0.9); 7.574 (1.2); 7.572 (1.2); 7.562 (1.2); 7.560 (1.2); 7.554 (1.2); 7.552
(1.2); 7.542 (1.2); 7.540 (1.1); 7.497 (1.6); 7.484 (3.0); 7.472 (1.5); 5.596 (7.3); 3.325 (13.6); 2.524 (0.6); 2.511 (12.4); 2.507 (24.2);
2.502 (31.2); 2.498 (22.2); 2.493 (10.5); 2.475 (16.0); 0.008 (0.4); 0.000 (9.9); −0.009 (0.3)
Compound No. I-1-195: ¹H NMR (400.0 MHz, d₆-DMSO):
10.993 (3.2); 9.094 (3.9); 8.933 (8.4); 8.928 (11.5); 8.715 (2.7); 8.703 (2.7); 8.311 (1.8); 8.306 (2.0); 8.291 (1.9); 8.286 (2.0); 7.517
(1.7); 7.504 (1.8); 7.497 (1.6); 7.485 (1.4); 6.533 (4.6); 5.449 (10.0); 3.329 (13.9); 3.324 (19.7); 2.507 (36.7); 2.502 (37.3);
2.312 (16.0); 0.004 (6.1); 0.000 (8.5)
Compound No. I-1-197: ¹H NMR(601.6 MHz, d₆-DMSO):
11.263 (4.2); 9.110 (3.8); 9.107 (3.8); 8.808 (12.6); 8.800 (12.9); 8.770 (2.1); 8.761 (2.2); 8.736 (3.2); 8.733 (3.3); 8.728 (3.4);
8.725 (3.3); 8.324 (1.6); 8.321 (2.3); 8.318 (1.6); 8.311 (1.7); 8.307 (2.4); 8.304 (1.7); 7.533 (2.2); 7.531 (2.2); 7.525 (2.2); 7.523
(2.2); 7.519 (2.2); 7.518 (2.2); 7.511 (2.2); 7.510 (2.1); 7.475 (2.8); 7.467 (5.5); 7.459 (2.7); 7.434 (0.5); 7.425 (1.0); 7.417 (0.5);
6.817 (8.5); 5.577 (2.2); 5.534 (16.0); 5.247 (3.3); 4.796 (1.3); 4.023 (0.3); 3.323 (51.2); 2.523 (0.4); 2.520 (0.5); 2.517 (0.5);
2.508 (15.4); 2.505 (34.4); 2.502 (48.7); 2.499 (35.7); 2.496 (17.1); 1.989 (1.4); 1.187 (0.4); 1.175 (0.7); 1.163 (0.4); 0.000 (11.6);
−0.006 (0.5)
Compound No. I-1-198: ¹H NMR (400.0 MHz, d₆-DMSO):
11.065 (1.5); 9.117 (1.9); 9.112 (1.9); 8.734 (1.4); 8.730 (1.5); 8.722 (1.5); 8.718 (1.5); 8.332 (0.8); 8.327 (1.2); 8.322 (0.8); 8.312
(0.8); 8.307 (1.2); 8.302 (0.8); 7.631 (2.5); 7.625 (2.6); 7.539 (1.2); 7.527 (1.1); 7.519 (1.1); 7.507 (1.1); 6.607 (2.2); 6.601
(2.3); 3.793 (16.0); 3.553 (0.5); 3.324 (5.3); 2.511 (6.5); 2.507 (13.3); 2.502 (17.8); 2.498 (13.2); 2.494 (6.6); 0.008 (0.6); 0.000
(17.0); −0.009 (0.7)
Compound No. I-1-199: ¹H NMR (400.0 MHz, d₆-DMSO):
10.516 (1.0); 10.429 (0.5); 9.126 (2.8); 8.769 (1.4); 8.765 (1.6); 8.757 (2.3); 8.753 (2.5); 8.744 (1.2); 8.325 (1.5); 8.316 (1.4); 8.305
(1.7); 8.270 (0.4); 7.640 (3.6); 7.571 (1.2); 7.559 (2.0); 7.551 (2.3); 7.541 (2.5); 7.527 (0.8); 7.502 (0.5); 7.490 (0.7); 7.412
(0.5); 5.399 (9.6); 5.344 (0.4); 4.981 (1.3); 4.903 (0.6); 4.879 (0.8); 4.645 (1.1); 4.590 (0.6); 3.685 (2.7); 3.667 (0.5); 3.328 (10.6);
2.777 (0.4); 2.758 (0.5); 2.739 (0.4); 2.675 (0.5); 2.671 (0.7); 2.667 (0.5); 2.506 (81.4); 2.502 (106.4); 2.497 (77.9); 2.333 (0.5);
2.329 (0.7); 2.324 (0.5); 1.942 (0.6); 1.899 (16.0); 1.858 (1.4); 1.848 (1.5); 1.810 (0.6); 1.492 (0.4); 1.327 (0.4); 1.309 (0.3);
0.903 (0.8); 0.885 (1.5); 0.867 (0.7); 0.146 (0.7); 0.008 (6.2); 0.000 (143.9); −0.009 (6.6); −0.150 (0.7)
Compound No. I-1-200: ¹H NMR (400.0 MHz, d₆-DMSO):
10.478 (1.8); 8.995 (1.1); 8.761 (1.0); 8.757 (1.0); 8.749 (1.0); 8.745 (1.0); 8.189 (0.6); 8.169 (0.6); 7.559 (0.8); 7.547 (0.8); 7.540
(0.8); 7.528 (0.7); 7.436 (2.6); 7.419 (1.0); 7.414 (2.9); 7.406 (0.4); 7.021 (0.4); 7.012 (4.0); 7.006 (1.2); 6.995 (1.2); 6.989
(3.6); 6.981 (0.4); 6.282 (3.0); 5.755 (2.2); 3.823 (1.1); 3.797 (0.7); 3.789 (0.4); 3.780 (0.5); 3.761 (16.0); 3.324 (18.8); 2.524 (0.5);
2.511 (11.1); 2.506 (22.6); 2.502 (29.8); 2.497 (21.2); 2.493 (10.0); 2.240 (10.5); 2.210 (0.7); 1.989 (0.7); 1.719 (0.3); 1.429
(0.3); 1.175 (0.4); 0.008 (0.6); 0.000 (19.3); −0.009 (0.6)
Compound No. I-1-201: ¹H NMR (400.0 MHz, d₆-DMSO):
10.541 (1.8); 9.114 (1.3); 9.111 (1.3); 8.791 (1.3); 8.787 (1.4); 8.779 (1.4); 8.775 (1.3); 8.308 (0.8); 8.288 (0.8); 7.591 (0.9); 7.590
(0.9); 7.579 (1.0); 7.578 (0.9); 7.572 (0.9); 7.560 (0.9); 3.919 (14.7); 3.326 (6.4); 2.512 (4.9); 2.507 (9.9); 2.503 (12.9); 2.498
(9.2); 2.494 (4.4); 2.402 (16.0); 0.000 (4.9)
Compound No. I-1-202: ¹H NMR (400.0 MHz, d₆-DMSO):
11.270 (3.1); 9.150 (2.0); 8.751 (1.5); 8.742 (1.5); 8.369 (1.1); 8.365 (1.7); 8.360 (1.1); 8.349 (1.2); 8.344 (1.7); 8.340 (1.1); 7.603
(0.8); 7.583 (1.9); 7.566 (1.5); 7.561 (2.2); 7.546 (2.1); 7.540 (1.5); 7.528 (1.4); 7.483 (0.9); 7.478 (1.7); 7.472 (1.2); 7.453
(3.5); 7.450 (3.7); 7.433 (1.5); 7.432 (1.6); 7.429 (1.3); 7.286 (0.8); 7.285 (0.8); 7.280 (0.8); 7.265 (1.4); 7.260 (1.4); 7.257 (1.1);
7.244 (0.7); 7.242 (0.7); 7.238 (0.7); 6.785 (4.4); 3.359 (2.1); 2.525 (0.6); 2.512 (12.2); 2.508 (24.0); 2.503 (31.1); 2.499 (22.2);
2.494 (10.6); 2.420 (16.0); 2.379 (0.4); 1.989 (1.1); 1.175 (0.6); 0.008 (1.8); 0.000 (40.6); −0.009 (1.5)
Compound No. I-1-203: ¹H NMR (400.0 MHz, d₆-DMSO):
11.232 (3.0); 9.147 (2.4); 9.143 (2.5); 8.750 (1.7); 8.746 (1.9); 8.738 (1.9); 8.734 (1.9); 8.367 (1.1); 8.362 (1.6); 8.357 (1.1); 8.347
(1.2); 8.342 (1.7); 8.337 (1.2); 7.615 (2.7); 7.610 (1.0); 7.603 (2.9); 7.598 (1.7); 7.593 (3.5); 7.586 (1.2); 7.581 (3.4); 7.572
(0.4); 7.556 (1.6); 7.544 (1.5); 7.536 (1.5); 7.526 (1.4); 7.524 (1.4); 7.391 (3.5); 7.386 (1.1); 7.379 (0.5); 7.374 (1.1); 7.369 (5.5);
7.364 (1.2); 7.360 (0.4); 7.353 (0.9); 7.347 (2.9); 6.750 (4.3); 3.389 (0.6); 2.525 (0.6); 2.520 (0.9); 2.512 (14.1); 2.507 (29.4);
2.503 (39.1); 2.498 (28.2); 2.494 (13.5); 2.347 (16.0); 2.330 (0.4); 2.239 (1.1); 0.008 (1.9); 0.000 (57.8); −0.009 (2.2)
Compound No. I-1-204: ¹H NMR (400.0 MHz, d₆-DMSO):
10.997 (2.8); 9.108 (2.3); 9.104 (2.2); 9.075 (0.5); 9.072 (0.5); 8.714 (1.6); 8.711 (1.7); 8.703 (1.6); 8.328 (1.1); 8.324 (1.6); 8.319
(1.1); 8.308 (1.2); 8.304 (1.6); 8.299 (1.0); 8.279 (0.4); 8.259 (0.4); 7.552 (0.3); 7.532 (0.3); 7.517 (1.5); 7.505 (1.5); 7.498
(1.5); 7.486 (1.3); 6.429 (4.3); 4.081 (0.4); 4.072 (0.5); 4.054 (0.9); 4.044 (1.0); 4.038 (0.7); 4.026 (0.6); 4.016 (0.6); 3.329 (27.8);
2.507 (29.6); 2.503 (36.7); 2.498 (26.3); 2.276 (16.0); 2.205 (0.4); 1.989 (0.6); 1.826 (4.1); 1.805 (3.1); 1.779 (2.2); 1.770 (1.5);
1.750 (1.5); 1.742 (1.3); 1.718 (0.7); 1.694 (0.9); 1.661 (1.0); 1.463 (0.5); 1.429 (1.4); 1.405 (1.3); 1.397 (1.5); 1.373 (0.5); 1.366
(0.5); 1.212 (0.5); 1.198 (1.6); 1.193 (0.6); 1.180 (1.0); 1.175 (1.0); 1.157 (0.7); 1.152 (0.7); 1.147 (0.8); 0.008 (3.4); 0.000
(45.0); −0.008 (1.9)
Compound No. I-1-205: ¹H NMR (400.0 MHz, d₆-DMSO):
11.034 (2.4); 9.111 (2.1); 9.107 (2.1); 8.714 (1.5); 8.710 (1.6); 8.702 (1.6); 8.699 (1.5); 8.333 (1.0); 8.328 (1.4); 8.324 (1.0); 8.313
(1.1); 8.309 (1.5); 8.304 (0.9); 7.516 (1.4); 7.504 (1.4); 7.496 (1.3); 7.484 (1.3); 6.436 (4.1); 4.214 (0.5); 4.200 (0.7); 4.197
(0.7); 4.191 (0.7); 4.184 (0.7); 4.177 (0.7); 4.161 (0.5); 4.038 (1.0); 4.020 (1.0); 4.002 (0.3); 3.328 (24.2); 2.671 (0.4); 2.524 (1.0);
2.511 (24.4); 2.506 (49.8); 2.502 (65.8); 2.497 (47.2); 2.493 (22.5); 2.329 (0.4); 2.324 (0.3); 2.267 (16.0); 1.989 (4.3); 1.859
(0.4); 1.841 (0.6); 1.837 (0.5); 1.825 (0.8); 1.819 (0.6); 1.807 (0.8); 1.803 (0.8); 1.784 (0.7); 1.725 (0.6); 1.712 (0.8); 1.707 (0.8);
1.694 (0.9); 1.678 (0.5); 1.673 (0.6); 1.660 (0.4); 1.363 (8.8); 1.347 (8.8); 1.193 (1.2); 1.175 (2.3); 1.157 (1.1); 0.729 (4.1); 0.710
(9.0); 0.692 (3.9); 0.146 (0.4); 0.008 (3.0); 0.000 (88.3); −0.009 (3.2); −0.014 (0.6); −0.150 (0.4)
Compound No. I-1-206: ¹H NMR (400.0 MHz, d₆-DMSO):
11.285 (3.4); 9.148 (3.2); 9.144 (3.1); 8.754 (2.1); 8.750 (2.2); 8.742 (2.2); 8.738 (2.1); 8.365 (1.2); 8.360 (1.8); 8.356 (1.2); 8.345
(1.4); 8.340 (1.9); 7.895 (3.7); 7.889 (3.7); 7.805 (3.2); 7.783 (4.0); 7.623 (2.3); 7.617 (2.1); 7.601 (1.9); 7.595 (1.7); 7.558
(1.6); 7.546 (1.7); 7.538 (1.6); 7.526 (1.5); 6.797 (4.7); 3.569 (0.6); 3.328 (30.0); 2.507 (36.0); 2.503 (43.7); 2.499 (32.3); 2.430
(16.0); 0.000 (50.6)
Compound No. I-1-207: ¹H NMR (400.0 MHz, d₆-DMSO):
11.223 (3.1); 9.135 (2.8); 9.131 (2.8); 8.744 (2.0); 8.740 (2.2); 8.732 (2.1); 8.728 (2.1); 8.352 (1.1); 8.348 (1.6); 8.343 (1.1); 8.333
(1.2); 8.328 (1.7); 8.323 (1.1); 7.939 (2.6); 7.936 (4.1); 7.933 (2.5); 7.633 (9.0); 7.630 (9.4); 7.549 (1.6); 7.537 (1.6); 7.529
(1.6); 7.517 (1.5); 6.760 (4.3); 5.757 (2.2); 3.328 (25.3); 2.512 (13.7); 2.507 (26.5); 2.503 (34.1); 2.498 (24.4); 2.494 (11.7); 2.126
(16.0); 1.234 (0.5); 0.008 (1.5); 0.000 (33.4); −0.009 (1.1)
Compound No. I-1-208: ¹H NMR (400.0 MHz, d₆-DMSO):
11.200 (1.6); 9.145 (1.4); 9.140 (1.4); 8.745 (1.1); 8.741 (1.2); 8.733 (1.1); 8.729 (1.1); 8.362 (0.6); 8.357 (0.9); 8.352 (0.6); 8.342
(0.6); 8.337 (0.9); 8.332 (0.6); 7.550 (0.8); 7.549 (0.8); 7.537 (0.8); 7.530 (0.8); 7.529 (0.8); 7.518 (0.7); 7.517 (0.7); 7.158
(3.5); 7.039 (1.6); 6.727 (2.2); 5.756 (1.2); 3.325 (20.6); 2.525 (0.5); 2.511 (9.9); 2.507 (19.9); 2.502 (26.0); 2.498 (18.6); 2.493
(8.7); 2.361 (8.5); 2.342 (16.0); 1.989 (0.4); 0.008 (2.5); 0.000 (56.5); −0.009 (2.0)
Compound No. I-1-209: ¹H NMR (400.0 MHz, d₆-DMSO):
11.264 (2.3); 9.146 (2.2); 9.142 (2.2); 8.749 (1.6); 8.745 (1.7); 8.737 (1.7); 8.733 (1.7); 8.363 (0.9); 8.359 (1.2); 8.354 (0.9); 8.343
(0.9); 8.338 (1.3); 8.334 (0.9); 7.619 (0.3); 7.610 (0.4); 7.596 (16.0); 7.581 (0.5); 7.573 (0.5); 7.553 (1.2); 7.552 (1.2); 7.541
(1.2); 7.540 (1.2); 7.534 (1.2); 7.532 (1.2); 7.522 (1.1); 7.520 (1.1); 7.445 (0.3); 6.773 (3.2); 3.325 (15.2); 2.525 (0.6); 2.507 (31.2);
2.502 (40.2); 2.498 (29.3); 2.385 (11.6); 2.384 (11.4); 1.989 (0.5); 0.146 (0.4); 0.008 (3.8); 0.000 (80.5); −0.009 (3.3); −0.150
(0.4)
Compound No. I-1-210: ¹H NMR (400.0 MHz, d₆-DMSO):
11.059 (2.5); 9.112 (2.4); 9.108 (2.4); 8.712 (1.7); 8.708 (1.8); 8.700 (1.8); 8.697 (1.8); 8.336 (1.0); 8.331 (1.4); 8.326 (1.0); 8.316
(1.1); 8.311 (1.5); 8.307 (1.0); 7.514 (1.4); 7.502 (1.4); 7.494 (1.4); 7.482 (1.3); 6.450 (4.1); 5.146 (0.5); 4.389 (0.3); 3.948
(0.6); 3.935 (0.8); 3.923 (1.1); 3.912 (0.8); 3.899 (0.6); 3.324 (26.1); 2.511 (17.0); 2.507 (33.3); 2.502 (43.1); 2.498 (31.5); 2.263
(15.2); 2.084 (1.9); 1.989 (0.9); 1.850 (0.6); 1.832 (0.9); 1.826 (0.7); 1.816 (1.4); 1.808 (1.0); 1.798 (1.4); 1.792 (1.5); 1.773
(1.6); 1.754 (1.6); 1.743 (1.5); 1.736 (1.6); 1.724 (1.7); 1.720 (1.4); 1.708 (1.0); 1.702 (1.0); 1.691 (0.7); 1.684 (0.3); 1.175 (0.5);
0.699 (7.5); 0.681 (16.0); 0.662 (7.1); 0.652 (1.4); 0.634 (2.2); 0.616 (1.0); 0.146 (0.3); 0.008 (3.0); 0.000 (69.3); −0.008
(2.9); −0.150 (0.3)
Compound No. I-1-211: ¹H NMR (600.1 MHz, d₆-DMSO):
10.607 (3.0); 10.491 (0.8); 9.112 (3.2); 9.099 (0.5); 9.095 (0.4); 8.813 (4.2); 8.808 (12.1); 8.805 (4.7); 8.800 (12.0); 8.792 (0.9);
8.784 (0.8); 8.759 (2.8); 8.757 (3.2); 8.751 (2.6); 8.749 (2.5); 8.311 (1.7); 8.298 (1.9); 7.560 (0.6); 7.554 (2.1); 7.546 (2.2); 7.541
(2.1); 7.533 (1.7); 7.476 (0.9); 7.469 (3.9); 7.461 (5.8); 7.453 (2.7); 7.442 (0.4); 6.533 (1.0); 5.545 (4.4); 5.457 (13.1); 5.429
(1.0); 4.036 (0.5); 4.025 (0.5); 3.337 (60.7); 2.522 (0.3); 2.519 (0.4); 2.510 (8.7); 2.507 (18.0); 2.504 (24.5); 2.501 (17.7); 2.498
(8.3); 2.324 (1.5); 2.300 (7.6); 2.274 (16.0); 1.990 (2.1); 1.187 (0.5); 1.175 (1.1); 1.164 (0.5); 0.000 (0.9)
Compound No. I-1-212: ¹H NMR (600.1 MHz, d₆-DMSO):
10.548 (0.8); 9.139 (0.7); 8.814 (3.9); 8.805 (4.1); 8.799 (0.8); 8.791 (0.8); 8.380 (0.4); 8.369 (0.4); 7.628 (0.5); 7.620 (0.5); 7.615
(0.5); 7.607 (0.4); 7.477 (0.9); 7.469 (1.8); 7.460 (0.9); 5.546 (4.3); 3.581 (1.0); 2.569 (12.5); 2.511 (2.4); 2.508 (5.0); 2.505
(6.8); 2.502 (4.9); 2.499 (2.3); 2.301 (7.2); 2.076 (16.0)
Compound No. I-1-213: ¹H NMR (400.0 MHz, d₆-DMSO):
11.195 (0.6); 9.138 (0.7); 9.133 (0.7); 8.744 (0.5); 8.740 (0.5); 8.732 (0.5); 8.728 (0.5); 8.350 (0.4); 8.330 (0.4); 7.730 (0.5); 7.711
(0.5); 7.706 (0.4); 7.586 (0.5); 7.582 (0.7); 7.578 (0.4); 7.572 (0.8); 7.568 (0.9); 7.565 (1.0); 7.558 (0.7); 7.553 (0.7); 7.549
(0.5); 7.541 (0.4); 7.537 (0.7); 7.529 (0.4); 7.528 (0.4); 7.517 (0.5); 7.277 (0.3); 6.749 (1.1); 6.681 (0.3); 3.331 (10.0); 2.512 (3.6);
2.508 (6.9); 2.503 (8.9); 2.499 (6.3); 2.494 (2.9); 2.115 (3.9); 1.756 (16.0); 0.008 (0.8); 0.000 (17.2); −0.009 (0.6)
Compound No. I-1-214: ¹H NMR (400.0 MHz, d₆-DMSO):
11.228 (2.8); 9.126 (1.7); 8.735 (1.3); 8.725 (1.3); 8.349 (1.1); 8.344 (1.6); 8.340 (1.1); 8.329 (1.2); 8.324 (1.7); 8.320 (1.1); 7.537
(1.4); 7.526 (1.4); 7.518 (1.4); 7.506 (1.3); 6.578 (4.3); 5.828 (0.7); 5.810 (2.6); 5.793 (2.7); 5.775 (0.8); 3.334 (4.3); 2.525
(0.5); 2.512 (10.7); 2.508 (21.6); 2.503 (28.3); 2.498 (20.7); 2.494 (10.4); 2.333 (16.0); 1.989 (0.7); 1.747 (9.4); 1.730 (9.3); 1.175
(0.4); 0.008 (2.0); 0.000 (51.6); −0.009 (2.5)
Compound No. I-1-215: ¹H NMR (400.0 MHz, d₆-DMSO):
11.128 (4.8); 9.201 (4.1); 9.197 (4.1); 8.781 (3.3); 8.777 (3.5); 8.769 (3.9); 8.761 (14.1); 8.749 (13.7); 8.401 (2.2); 8.381 (2.3);
7.813 (4.1); 7.792 (4.4); 7.670 (4.2); 7.649 (5.0); 7.587 (2.4); 7.574 (2.4); 7.567 (2.4); 7.555 (2.2); 7.443 (3.3); 7.431 (6.7); 7.419
(3.4); 7.410 (2.9); 7.407 (3.4); 7.389 (2.2); 7.387 (2.1); 7.150 (2.7); 7.132 (3.7); 7.114 (2.3); 5.845 (16.0); 3.327 (39.2); 2.671
(0.4); 2.506 (50.9); 2.502 (64.8); 2.497 (46.9); 2.328 (0.4); 0.008 (1.9); 0.000 (41.3); −0.008 (1.7)
Compound No. I-1-216: ¹H NMR (400.0 MHz, d₆-DMSO):
11.233 (4.1); 9.085 (3.9); 8.876 (0.4); 8.872 (0.8); 8.868 (0.4); 8.827 (0.9); 8.819 (4.8); 8.812 (4.6); 8.760 (15.2); 8.748 (15.5);
8.735 (1.0); 8.729 (0.9); 8.310 (1.9); 8.286 (2.0); 7.826 (4.1); 7.806 (4.6); 7.788 (0.5); 7.677 (4.5); 7.656 (5.3); 7.613 (0.4); 7.592
(0.6); 7.443 (3.7); 7.431 (9.1); 7.419 (4.2); 7.416 (3.6); 7.413 (3.6); 7.409 (2.5); 7.394 (2.5); 7.392 (2.4); 7.158 (2.8); 7.157 (2.8);
7.139 (3.6); 7.121 (2.5); 7.119 (2.3); 5.850 (16.0); 5.756 (2.9); 5.059 (1.4); 3.350 (0.4); 3.326 (62.7); 3.188 (0.4); 3.172 (0.4);
2.675 (0.4); 2.671 (0.6); 2.667 (0.4); 2.541 (0.4); 2.524 (1.4); 2.511 (32.7); 2.506 (66.0); 2.502 (86.0); 2.497 (60.5); 2.493 (28.0);
2.333 (0.4); 2.329 (0.5); 2.324 (0.4); 1.739 (0.3); 1.722 (0.5); 0.146 (0.8); 0.008 (6.9); 0.000 (177.6); −0.009 (6.0); −0.150
(0.8)
Compound No. I-1-217: ¹H NMR (400.0 MHz, d₆-DMSO):
11.290 (3.6); 9.148 (3.3); 9.144 (3.3); 8.753 (2.3); 8.744 (2.2); 8.361 (1.9); 8.341 (2.0); 8.086 (3.6); 7.952 (1.7); 7.931 (2.1); 7.892
(2.0); 7.872 (2.6); 7.766 (2.2); 7.746 (3.1); 7.726 (1.3); 7.601 (0.3); 7.561 (1.7); 7.549 (1.8); 7.541 (1.7); 7.529 (1.5); 6.809
(4.8); 3.330 (17.1); 2.503 (45.7); 2.436 (16.0); 2.392 (0.6); 2.330 (0.4); 1.990 (0.7); 1.233 (0.4); 1.193 (0.4); 1.175 (0.5); 0.000
(2.8)
Compound No. I-1-218: ¹H NMR (400.0 MHz, d₆-DMSO):
11.042 (2.6); 9.117 (2.5); 9.112 (2.5); 8.717 (1.8); 8.713 (1.9); 8.705 (1.9); 8.701 (1.9); 8.338 (1.0); 8.333 (1.5); 8.328 (1.0); 8.318
(1.1); 8.313 (1.6); 8.308 (1.0); 7.519 (1.4); 7.507 (1.4); 7.499 (1.4); 7.487 (1.3); 6.430 (4.4); 3.627 (1.1); 3.610 (1.2); 3.604
(1.2); 3.587 (1.1); 3.325 (9.3); 3.169 (1.5); 2.671 (0.4); 2.524 (1.0); 2.511 (22.0); 2.506 (44.0); 2.502 (57.8); 2.498 (41.8); 2.493
(20.5); 2.329 (0.4); 2.231 (16.0); 2.089 (0.9); 1.463 (8.1); 1.447 (7.9); 1.370 (0.6); 1.353 (0.6); 1.330 (0.4); 1.321 (0.5); 1.308
(0.9); 1.298 (0.8); 1.287 (0.9); 1.275 (0.5); 1.267 (0.4); 1.248 (0.4); 0.606 (0.3); 0.597 (0.5); 0.593 (0.5); 0.584 (1.1); 0.577 (0.9);
0.572 (0.9); 0.563 (1.1); 0.551 (0.6); 0.541 (0.5); 0.412 (0.4); 0.399 (0.6); 0.390 (1.0); 0.378 (0.9); 0.369 (1.3); 0.357 (1.0); 0.349
(1.1); 0.343 (0.9); 0.336 (1.2); 0.323 (1.4); 0.313 (1.2); 0.300 (0.5); 0.285 (0.6); 0.272 (1.0); 0.262 (1.2); 0.256 (0.7); 0.249
(1.1); 0.240 (0.7); 0.229 (0.4); 0.146 (0.3); 0.008 (3.0); 0.000 (74.1); −0.009 (3.0); −0.150 (0.4)
Compound No. I-1-219: ¹H NMR (400.0 MHz, d₆-DMSO):
11.128 (1.2); 9.109 (1.2); 9.104 (1.2); 8.734 (0.9); 8.730 (0.9); 8.722 (0.9); 8.718 (0.9); 8.330 (0.5); 8.325 (0.7); 8.320 (0.5); 8.310
(0.5); 8.306 (0.7); 8.301 (0.5); 7.534 (0.7); 7.522 (0.7); 7.514 (0.7); 7.502 (0.7); 6.653 (1.9); 3.329 (8.3); 2.515 (8.9); 2.507
(10.4); 2.503 (13.0); 2.499 (9.5); 1.989 (0.7); 1.974 (16.0); 0.000 (1.2)
Compound No. I-1-220: ¹H NMR (400.0 MHz, d₆-DMSO):
10.969 (4.1); 9.083 (3.9); 9.079 (3.9); 8.725 (2.8); 8.721 (3.1); 8.713 (3.0); 8.709 (3.0); 8.303 (1.5); 8.298 (2.3); 8.293 (1.6); 8.283
(1.6); 8.278 (2.4); 8.273 (1.6); 7.523 (2.3); 7.511 (2.2); 7.503 (2.2); 7.491 (2.1); 7.367 (5.4); 7.364 (5.5); 6.484 (6.1); 6.202
(16.0); 6.049 (5.1); 6.047 (5.1); 5.756 (0.5); 4.047 (0.6); 3.324 (36.3); 2.671 (0.4); 2.667 (0.3); 2.511 (25.6); 2.506 (51.6); 2.502
(68.2); 2.497 (50.5); 2.488 (48.9); 2.458 (0.4); 2.324 (0.6); 0.146 (0.4); 0.008 (3.1); 0.000 (85.5); −0.009 (3.3); −0.150 (0.4)
Compound No. I-1-221: ¹H NMR (400.0 MHz, d₆-DMSO):
11.274 (2.4); 9.127 (2.6); 9.122 (2.6); 8.753 (1.9); 8.749 (2.0); 8.741 (2.0); 8.737 (2.0); 8.342 (1.1); 8.337 (1.6); 8.332 (1.0); 8.322
(1.1); 8.317 (1.7); 8.312 (1.0); 7.557 (1.5); 7.545 (1.5); 7.537 (1.5); 7.525 (1.4); 7.453 (1.4); 7.320 (3.2); 7.186 (1.6); 6.969
(3.5); 3.873 (16.0); 3.333 (21.1); 2.526 (0.4); 2.513 (8.3); 2.508 (16.6); 2.504 (21.5); 2.499 (15.3); 2.495 (7.3); 0.000 (7.2)
Compound No. I-1-222: ¹H NMR (400.0 MHz, d₆-DMSO):
11.377 (2.5); 9.011 (3.3); 8.789 (3.1); 8.783 (3.2); 8.265 (1.0); 8.260 (1.3); 8.254 (1.0); 8.241 (1.0); 8.236 (1.3); 8.230 (1.0); 7.456
(1.4); 7.323 (3.1); 7.189 (1.5); 6.968 (3.6); 3.877 (16.0); 3.329 (10.5); 2.526 (0.4); 2.508 (16.8); 2.504 (21.8); 2.499 (15.7); 0.008
(0.4); 0.000 (9.1); −0.008 (0.4)
Compound No. I-1-223: ¹H NMR (400.0 MHz, d₆-DMSO):
11.123 (4.8); 9.090 (4.5); 9.085 (4.5); 8.726 (3.1); 8.723 (3.2); 8.714 (3.2); 8.711 (3.1); 8.309 (1.8); 8.305 (2.6); 8.300 (1.8); 8.289
(1.9); 8.285 (2.7); 8.280 (1.7); 8.210 (7.8); 7.782 (7.5); 7.525 (2.5); 7.513 (2.5); 7.505 (2.4); 7.493 (2.3); 6.631 (16.0); 6.561
(6.9); 6.343 (0.4); 3.330 (40.0); 2.672 (0.4); 2.507 (45.5); 2.503 (57.0); 2.498 (44.1); 2.493 (38.4); 2.329 (0.5); 2.307 (0.5); 0.000
(52.9)
Compound No. I-1-224: ¹H NMR (400.0 MHz, d₆-DMSO):
11.097 (4.9); 9.087 (4.6); 9.083 (4.6); 8.736 (10.5); 8.724 (3.3); 8.720 (3.5); 8.712 (3.2); 8.709 (3.1); 8.302 (2.8); 8.286 (2.0); 8.282
(2.8); 8.037 (9.7); 7.522 (2.6); 7.510 (2.6); 7.502 (2.5); 7.490 (2.3); 6.539 (7.3); 6.417 (16.0); 3.332 (41.9); 3.176 (0.4); 3.163
(0.4); 2.672 (0.3); 2.507 (40.7); 2.503 (51.7); 2.499 (42.0); 2.493 (39.1); 2.329 (0.5); 0.000 (3.7)
Compound No. I-1-225: ¹H NMR (400.0 MHz, d₆-DMSO):
11.088 (2.7); 9.094 (2.9); 9.089 (2.9); 8.724 (1.9); 8.720 (2.1); 8.712 (2.1); 8.708 (2.1); 8.313 (1.1); 8.308 (1.7); 8.304 (1.2); 8.293
(1.2); 8.288 (1.8); 8.284 (1.2); 7.876 (3.3); 7.870 (3.4); 7.518 (3.9); 7.514 (4.2); 7.503 (1.7); 7.491 (1.5); 6.515 (4.4); 6.301
(2.6); 6.296 (4.5); 6.288 (11.1); 3.339 (1.4); 2.507 (19.0); 2.503 (24.6); 2.498 (18.6); 2.477 (16.0); 0.000 (2.2)
Compound No. I-1-226: ¹H NMR (400.0 MHz, d₆-DMSO):
11.074 (2.7); 9.094 (2.5); 9.090 (2.6); 8.725 (1.9); 8.721 (2.0); 8.713 (2.0); 8.709 (2.0); 8.314 (1.1); 8.309 (1.6); 8.304 (1.1); 8.294
(1.2); 8.289 (1.6); 8.284 (1.1); 7.602 (4.3); 7.525 (1.5); 7.524 (1.5); 7.513 (1.5); 7.512 (1.5); 7.506 (1.5); 7.504 (1.5); 7.493
(1.4); 7.492 (1.4); 7.310 (5.1); 6.500 (4.1); 6.190 (10.2); 3.353 (1.3); 2.525 (0.7); 2.511 (14.1); 2.507 (28.7); 2.502 (37.9); 2.498
(27.4); 2.493 (13.3); 2.455 (15.5); 1.991 (16.0); 0.000 (4.0)
Compound No. I-1-227: ¹H NMR (400.0 MHz, d₆-DMSO):
11.027 (3.1); 9.075 (3.1); 9.071 (3.0); 8.717 (2.1); 8.714 (2.1); 8.706 (2.3); 8.702 (2.0); 8.295 (1.7); 8.275 (1.8); 7.757 (3.4); 7.515
(1.6); 7.503 (1.6); 7.495 (1.6); 7.483 (1.5); 6.979 (3.6); 6.557 (4.5); 6.381 (10.1); 3.329 (20.7); 2.506 (35.1); 2.502 (42.3); 2.498
(31.1); 2.477 (16.0); 2.329 (0.4); 1.234 (0.4); 0.000 (3.4)
Compound No. I-1-228: ¹H NMR (400.0 MHz, d₆-DMSO):
11.125 (3.3); 9.094 (2.8); 9.090 (2.8); 8.727 (1.9); 8.723 (2.0); 8.715 (2.0); 8.711 (1.9); 8.313 (1.1); 8.309 (1.7); 8.304 (1.1); 8.293
(1.2); 8.289 (1.8); 8.284 (1.1); 8.126 (2.7); 8.123 (2.8); 7.526 (1.6); 7.514 (1.6); 7.506 (1.6); 7.494 (1.5); 6.796 (3.2); 6.790
(3.2); 6.560 (4.6); 6.419 (9.8); 3.336 (6.4); 2.508 (23.3); 2.503 (29.9); 2.499 (21.8); 2.474 (16.0); 0.000 (2.8)
Compound No. I-1-229: ¹H NMR (400.0 MHz, d₆-DMSO):
11.036 (2.5); 9.145 (2.6); 9.141 (2.6); 9.090 (0.6); 9.089 (0.6); 9.085 (0.7); 8.816 (0.5); 8.812 (0.6); 8.804 (0.6); 8.800 (0.6); 8.761
(2.0); 8.757 (2.1); 8.748 (2.1); 8.744 (2.0); 8.421 (1.0); 8.417 (1.4); 8.412 (0.9); 8.401 (1.0); 8.397 (1.5); 8.392 (0.9); 8.320
(0.4); 8.316 (0.5); 8.300 (0.3); 8.296 (0.5); 7.606 (1.4); 7.594 (1.5); 7.586 (1.7); 7.574 (1.4); 7.567 (0.4); 6.441 (4.3); 4.651 (1.0);
4.632 (1.5); 4.614 (1.1); 4.594 (0.4); 2.526 (0.6); 2.512 (13.4); 2.508 (27.0); 2.503 (35.1); 2.499 (24.8); 2.494 (11.5); 2.282 (16.0);
2.060 (0.4); 2.041 (0.8); 2.031 (1.2); 2.024 (0.9); 2.010 (1.4); 2.000 (1.1); 1.938 (0.4); 1.921 (1.1); 1.902 (1.4); 1.889 (1.1);
1.884 (1.0); 1.871 (0.9); 1.853 (0.9); 1.836 (1.1); 1.821 (1.4); 1.813 (1.4); 1.803 (1.0); 1.780 (0.3); 1.659 (0.3); 1.650 (0.6); 1.640
(1.2); 1.631 (1.4); 1.625 (1.6); 1.611 (1.3); 1.604 (0.8); 0.000 (7.3)
Compound No. I-1-230: ¹H NMR (400.0 MHz, d₆-DMSO):
13.055 (2.3); 9.154 (2.4); 9.150 (2.3); 8.857 (1.7); 8.854 (1.8); 8.845 (1.8); 8.842 (1.7); 8.615 (1.4); 8.612 (1.4); 8.602 (1.4); 8.600
(1.4); 8.347 (1.0); 8.342 (1.3); 8.337 (1.0); 8.327 (1.1); 8.321 (1.4); 8.317 (1.1); 8.084 (0.7); 8.080 (0.7); 8.066 (1.0); 8.063
(1.4); 8.059 (1.3); 8.045 (1.2); 8.040 (1.2); 7.988 (2.5); 7.967 (1.5); 7.684 (1.2); 7.683 (1.2); 7.672 (1.2); 7.671 (1.2); 7.664 (1.2);
7.663 (1.2); 7.653 (1.1); 7.651 (1.1); 7.394 (1.1); 7.392 (1.1); 7.382 (1.1); 7.379 (1.3); 7.376 (1.3); 7.374 (1.1); 7.364 (1.1); 7.361
(1.0); 6.784 (5.2); 3.327 (45.5); 2.525 (0.8); 2.512 (14.4); 2.508 (28.0); 2.503 (36.2); 2.499 (26.1); 2.494 (12.6); 2.284 (16.0);
0.000 (5.4)
Compound No. I-1-231: ¹H NMR(601.6 MHz, d₆-DMSO):
11.416 (1.2); 9.131 (1.2); 9.128 (1.2); 8.758 (0.9); 8.752 (0.8); 8.750 (0.9); 8.347 (0.7); 8.333 (0.7); 7.556 (0.6); 7.549 (0.6); 7.544
(0.7); 7.535 (0.6); 6.816 (1.7); 5.414 (0.8); 3.345 (32.9); 2.911 (16.0); 2.615 (0.4); 2.602 (0.3); 2.504 (25.1); 2.478 (6.3); 2.391
(1.2); 1.178 (0.3); 1.166 (0.7); 1.154 (0.3); 0.000 (13.4)
Compound No. I-1-232: ¹H NMR (400.0 MHz, d₆-DMSO):
9.926 (0.8); 8.494 (2.9); 8.485 (2.9); 8.482 (2.8); 8.422 (2.5); 7.640 (1.4); 7.621 (1.6); 7.371 (5.3); 7.365 (5.5); 7.352 (2.4); 7.340
(2.4); 7.333 (2.2); 7.320 (1.9); 7.070 (2.5); 7.064 (2.5); 7.049 (2.8); 7.043 (2.7); 6.662 (4.2); 6.641 (3.8); 6.491 (0.5); 6.443 (0.4);
5.756 (10.0); 4.055 (0.3); 4.038 (1.0); 4.020 (1.0); 4.002 (0.3); 3.744 (0.9); 3.666 (1.4); 3.632 (16.0); 3.600 (0.5); 3.326 (51.8);
2.671 (0.4); 2.667 (0.3); 2.506 (53.6); 2.502 (69.4); 2.497 (52.1); 2.329 (0.5); 2.324 (0.4); 2.300 (0.7); 2.268 (1.0); 2.254 (1.4);
2.223 (15.3); 1.989 (4.3); 1.193 (1.2); 1.175 (2.3); 1.157 (1.1); 0.146 (0.4); 0.008 (3.6); 0.000 (74.7); −0.008 (3.6); −0.150
(0.4)
Compound No. I-1-233: ¹H NMR (400.0 MHz, d₆-DMSO):
11.659 (4.5); 9.174 (3.4); 9.170 (3.6); 8.770 (2.4); 8.766 (2.7); 8.758 (2.7); 8.754 (2.7); 8.396 (1.4); 8.391 (2.0); 8.386 (1.5); 8.376
(1.6); 8.371 (2.1); 8.366 (1.6); 7.570 (1.8); 7.568 (1.8); 7.557 (1.8); 7.556 (1.8); 7.550 (1.8); 7.548 (1.8); 7.537 (1.8); 7.536
(1.8); 7.524 (2.3); 7.519 (1.2); 7.506 (4.0); 7.503 (4.6); 7.490 (1.5); 7.485 (4.2); 7.391 (6.4); 7.380 (2.1); 7.375 (3.3); 7.372 (4.4);
7.360 (3.1); 7.344 (0.9); 7.342 (1.3); 6.972 (4.8); 6.970 (4.9); 6.757 (0.4); 6.755 (0.4); 6.736 (0.4); 6.616 (0.4); 3.324 (30.4); 2.671
(0.4); 2.587 (15.9); 2.585 (16.0); 2.525 (0.8); 2.511 (20.9); 2.507 (42.9); 2.502 (57.6); 2.498 (44.0); 2.494 (23.3); 2.366 (1.1);
2.329 (0.4); 0.008 (2.0); 0.000 (52.1); −0.008 (2.8)
Compound No. I-1-234: ¹H NMR (400.0 MHz, d₆-DMSO):
11.113 (3.1); 9.100 (2.8); 9.096 (2.8); 8.723 (11.5); 8.719 (3.3); 8.711 (11.6); 8.320 (1.2); 8.315 (1.7); 8.310 (1.2); 8.300 (1.3);
8.295 (1.8); 8.290 (1.2); 7.523 (1.6); 7.522 (1.6); 7.511 (1.5); 7.510 (1.6); 7.503 (1.5); 7.502 (1.5); 7.491 (1.4); 7.490 (1.5); 7.317
(2.7); 7.305 (5.0); 7.292 (2.5); 6.508 (4.4); 5.893 (11.7); 3.330 (28.3); 2.512 (11.0); 2.508 (21.5); 2.503 (28.1); 2.499 (20.4); 2.495
(10.0); 2.411 (16.0); 0.008 (0.6); 0.000 (14.2); −0.008 (0.5)
Compound No. I-1-235: ¹H NMR (600.1 MHz, d₆-DMSO):
11.077 (3.4); 9.100 (2.9); 9.098 (3.0); 9.036 (10.0); 9.028 (10.2); 8.740 (2.3); 8.737 (2.4); 8.732 (2.4); 8.729 (2.4); 8.317 (1.2);
8.314 (1.7); 8.311 (1.2); 8.304 (1.2); 8.301 (1.7); 8.297 (1.2); 7.900 (0.4); 7.738 (2.7); 7.730 (5.2); 7.721 (2.7); 7.540 (1.7); 7.539
(1.7); 7.532 (1.7); 7.531 (1.6); 7.527 (1.7); 7.525 (1.6); 7.519 (1.7); 7.517 (1.6); 6.582 (4.1); 5.613 (2.8); 5.591 (4.3); 5.523 (4.2);
5.501 (2.9); 5.447 (0.5); 3.380 (1.1); 3.356 (38.8); 2.513 (5.2); 2.510 (11.1); 2.507 (15.3); 2.504 (11.2); 2.501 (5.3); 2.377 (0.7);
2.313 (16.0); 1.059 (0.4)
Compound No. I-1-236: ¹H NMR(601.6 MHz, d₆-DMSO):
11.031 (3.5); 9.141 (9.9); 9.133 (10.0); 9.049 (2.8); 9.045 (2.7); 8.722 (2.3); 8.719 (2.5); 8.714 (2.4); 8.711 (2.5); 8.706 (0.4); 8.698
(0.3); 8.270 (1.1); 8.266 (1.6); 8.263 (1.2); 8.256 (1.2); 8.253 (1.7); 8.250 (1.2); 7.924 (2.8); 7.916 (5.3); 7.908 (2.7); 7.517
(1.5); 7.516 (1.6); 7.509 (1.5); 7.508 (1.6); 7.503 (1.5); 7.502 (1.6); 7.495 (1.4); 7.494 (1.6); 6.609 (3.9); 6.061 (11.3); 5.503 (0.4);
3.373 (0.5); 3.358 (2.8); 3.349 (346.5); 3.336 (0.9); 3.332 (0.5); 2.618 (0.4); 2.615 (0.5); 2.612 (0.4); 2.524 (0.9); 2.521 (1.1);
2.518 (1.2); 2.509 (26.1); 2.506 (57.1); 2.503 (79.4); 2.500 (58.7); 2.497 (27.7); 2.391 (0.4); 2.388 (0.5); 2.385 (0.4); 2.360 (16.0);
2.276 (0.5); 0.000 (5.5)
Compound No. I-1-237: ¹H NMR(601.6 MHz, d₆-DMSO):
11.068 (3.0); 9.115 (2.8); 9.112 (2.8); 8.726 (2.0); 8.724 (2.2); 8.718 (2.2); 8.716 (2.2); 8.328 (1.6); 8.315 (1.6); 7.527 (1.5); 7.519
(1.5); 7.514 (1.5); 7.506 (1.5); 6.506 (4.3); 4.328 (1.2); 4.316 (1.2); 4.303 (1.4); 4.291 (1.4); 4.065 (1.6); 4.054 (1.7); 4.040
(1.4); 4.029 (1.4); 3.347 (11.7); 3.344 (25.7); 2.507 (21.0); 2.504 (29.2); 2.501 (22.4); 2.318 (16.0); 2.296 (0.5); 2.284 (0.9); 2.278
(0.5); 2.271 (1.0); 2.266 (1.0); 2.260 (0.5); 2.254 (0.9); 2.242 (0.4); 1.990 (0.7); 1.863 (1.6); 1.851 (1.8); 1.845 (1.6); 1.833
(1.5); 1.590 (1.6); 1.577 (2.8); 1.564 (1.4); 1.175 (0.3); 0.000 (27.6)
Compound No. I-1-238: ¹H NMR(601.6 MHz, d₆-DMSO):
11.079 (2.8); 9.110 (2.6); 9.107 (2.6); 8.725 (2.0); 8.722 (2.1); 8.717 (2.2); 8.714 (2.2); 8.326 (0.9); 8.323 (1.5); 8.320 (1.0); 8.313
(1.0); 8.309 (1.5); 8.306 (1.0); 7.526 (1.5); 7.518 (1.5); 7.513 (1.5); 7.505 (1.4); 6.508 (4.2); 6.381 (1.5); 6.370 (3.4); 6.359
(1.6); 4.777 (6.2); 4.765 (6.1); 3.349 (9.4); 3.345 (11.6); 3.342 (14.4); 3.173 (1.2); 3.165 (1.2); 2.522 (0.3); 2.519 (0.4); 2.510 (9.8);
2.507 (21.2); 2.504 (29.0); 2.501 (20.8); 2.498 (9.4); 2.301 (16.0); 0.005 (1.0); 0.000 (31.4); −0.006 (1.0)
Compound No. I-1-239: ¹H NMR(601.6 MHz, d₆-DMSO):
11.011 (2.9); 9.108 (2.7); 9.105 (2.8); 8.719 (1.9); 8.717 (2.1); 8.711 (2.0); 8.709 (2.2); 8.319 (1.6); 8.306 (1.6); 7.520 (1.5); 7.512
(1.5); 7.507 (1.6); 7.499 (1.5); 6.453 (4.4); 5.269 (0.7); 5.258 (1.4); 5.246 (0.7); 4.600 (3.2); 4.589 (3.1); 3.352 (8.7); 3.348
(11.0); 3.343 (11.2); 2.507 (20.9); 2.504 (28.7); 2.501 (21.6); 2.254 (16.0); 2.234 (0.3); 1.747 (11.9); 1.707 (10.5); 0.000 (25.7);
−0.006 (0.9)
Compound No. I-1-240: ¹H NMR (400.0 MHz, d₆-DMSO):
11.417 (0.4); 9.618 (3.9); 9.616 (3.9); 9.613 (3.3); 9.445 (3.2); 9.442 (3.2); 9.431 (3.3); 9.429 (3.3); 8.122 (2.7); 8.116 (2.8); 8.109
(2.8); 8.103 (2.7); 7.879 (4.6); 7.873 (4.6); 7.516 (4.9); 7.513 (4.9); 6.528 (6.5); 6.301 (16.0); 6.295 (7.1); 6.290 (4.1); 3.330
(12.6); 2.891 (0.4); 2.731 (0.4); 2.671 (0.4); 2.507 (44.4); 2.502 (57.4); 2.498 (44.3); 2.484 (23.6); 2.329 (0.4); 2.324 (0.4); 2.295
(0.4); 0.000 (65.0)
Compound No. I-1-241: ¹H NMR (400.0 MHz, d₆-DMSO):
11.073 (1.7); 9.114 (1.8); 9.109 (1.8); 8.729 (1.5); 8.725 (1.6); 8.717 (1.6); 8.713 (1.6); 8.333 (0.7); 8.328 (1.1); 8.323 (0.8); 8.313
(0.8); 8.308 (1.1); 8.303 (0.8); 7.532 (1.1); 7.531 (1.1); 7.520 (1.1); 7.519 (1.1); 7.512 (1.1); 7.511 (1.1); 7.500 (1.0); 7.499
(1.0); 6.542 (2.8); 5.157 (8.4); 4.915 (0.4); 3.331 (21.5); 2.521 (0.4); 2.512 (5.6); 2.508 (11.9); 2.503 (15.9); 2.499 (11.7); 2.494
(5.8); 2.320 (11.3); 2.143 (16.0); 2.100 (0.8); 0.000 (1.2)
Compound No. I-1-242: ¹H NMR (400.0 MHz, d₆-DMSO):
11.059 (2.5); 9.111 (2.5); 9.107 (2.6); 8.728 (2.1); 8.724 (2.3); 8.716 (2.3); 8.712 (2.3); 8.332 (1.0); 8.327 (1.5); 8.322 (1.1); 8.312
(1.1); 8.307 (1.6); 8.302 (1.1); 7.531 (1.5); 7.529 (1.6); 7.519 (1.5); 7.517 (1.6); 7.511 (1.5); 7.509 (1.6); 7.499 (1.4); 7.497
(1.5); 6.538 (3.9); 5.286 (0.4); 5.194 (12.0); 4.953 (1.4); 4.513 (0.4); 3.331 (31.7); 2.653 (1.6); 2.635 (5.1); 2.616 (5.4); 2.598 (2.3);
2.580 (0.7); 2.521 (0.4); 2.512 (7.3); 2.508 (15.5); 2.503 (20.9); 2.499 (15.5); 2.494 (7.8); 2.337 (0.4); 2.321 (16.0); 2.142 (1.5);
2.141 (1.6); 1.199 (6.5); 1.181 (13.8); 1.162 (6.9); 1.142 (0.7); 0.000 (1.6)
Compound No. I-1-243: ¹H NMR (400.0 MHz, d₆-DMSO):
11.124 (2.4); 9.117 (2.2); 9.113 (2.3); 8.738 (1.5); 8.734 (1.8); 8.726 (1.7); 8.722 (1.7); 8.336 (0.9); 8.331 (1.3); 8.327 (1.0); 8.316
(1.0); 8.311 (1.4); 8.307 (1.0); 7.540 (1.3); 7.528 (1.3); 7.521 (1.2); 7.520 (1.3); 7.509 (1.1); 7.508 (1.2); 6.575 (3.5); 5.373
(2.1); 5.340 (3.1); 5.226 (3.2); 5.192 (2.1); 3.329 (22.8); 2.651 (16.0); 2.507 (17.5); 2.503 (23.7); 2.498 (18.3); 2.340 (12.9); 0.008
(0.8); 0.000 (19.8)
Compound No. I-1-244: ¹H NMR (400.0 MHz, d₆-DMSO):
11.148 (3.3); 9.114 (2.9); 9.109 (3.1); 8.742 (2.0); 8.738 (2.3); 8.730 (2.2); 8.726 (2.2); 8.332 (1.1); 8.328 (1.7); 8.323 (1.3); 8.312
(1.2); 8.308 (1.8); 8.303 (1.3); 7.543 (1.6); 7.531 (1.6); 7.524 (1.6); 7.512 (1.5); 7.511 (1.5); 6.615 (4.5); 5.605 (8.7); 3.332
(26.5); 3.074 (16.0); 2.508 (14.3); 2.503 (19.0); 2.499 (14.9); 2.352 (15.9); 1.990 (0.8); 1.175 (0.4); 0.008 (0.6); 0.000 (15.6)
Compound No. I-1-245: ¹H NMR (400.0 MHz, d₆-DMSO):
11.119 (3.1); 9.117 (2.9); 9.112 (2.9); 8.738 (2.1); 8.734 (2.3); 8.726 (2.2); 8.722 (2.3); 8.336 (1.1); 8.331 (1.7); 8.326 (1.2); 8.316
(1.3); 8.311 (1.8); 8.306 (1.2); 7.541 (1.5); 7.539 (1.6); 7.527 (1.6); 7.521 (1.5); 7.520 (1.6); 7.509 (1.4); 7.507 (1.5); 6.572
(4.3); 5.367 (2.6); 5.333 (4.0); 5.321 (0.3); 5.227 (3.9); 5.193 (2.6); 4.368 (0.4); 4.355 (0.8); 4.342 (0.4); 3.456 (0.6); 3.444 (0.5);
3.439 (0.6); 3.426 (0.5); 3.334 (52.9); 3.176 (0.7); 3.163 (0.7); 2.960 (1.0); 2.945 (0.6); 2.941 (1.2); 2.927 (1.7); 2.908 (1.7); 2.889
(0.6); 2.847 (0.5); 2.829 (1.6); 2.810 (1.7); 2.795 (1.1); 2.792 (0.8); 2.777 (1.0); 2.525 (0.4); 2.512 (8.5); 2.508 (17.3); 2.503
(23.1); 2.499 (17.6); 2.339 (16.0); 2.168 (0.9); 1.253 (5.4); 1.235 (11.3); 1.216 (5.3); 1.195 (0.6); 1.074 (1.2); 1.056 (2.2); 1.039
(1.1); 0.008 (0.8); 0.000 (21.7); −0.008 (1.1)
Compound No. I-1-246: ¹H NMR (400.0 MHz, d₆-DMSO):
11.129 (3.1); 9.110 (2.8); 9.105 (2.9); 8.741 (2.1); 8.737 (2.3); 8.729 (2.3); 8.725 (2.3); 8.329 (1.1); 8.324 (1.7); 8.320 (1.2); 8.309
(1.2); 8.304 (1.8); 8.300 (1.2); 7.543 (1.6); 7.541 (1.6); 7.531 (1.6); 7.529 (1.6); 7.523 (1.6); 7.522 (1.6); 7.511 (1.5); 7.509
(1.5); 6.607 (4.4); 5.601 (9.9); 4.038 (0.8); 4.020 (0.8); 3.332 (50.6); 3.221 (1.3); 3.202 (4.1); 3.184 (4.2); 3.165 (1.4); 2.512 (9.9);
2.507 (20.5); 2.503 (27.4); 2.498 (20.6); 2.494 (10.6); 2.360 (16.0); 2.335 (0.3); 1.989 (3.6); 1.280 (5.5); 1.261 (11.9); 1.242
(5.5); 1.193 (1.0); 1.175 (1.9); 1.157 (0.9)
Compound No. I-1-247: ¹H NMR (400.0 MHz, d₆-DMSO):
11.009 (3.3); 9.105 (2.8); 9.100 (2.8); 8.726 (1.9); 8.722 (2.1); 8.714 (2.1); 8.710 (2.1); 8.322 (1.1); 8.317 (1.7); 8.312 (1.2); 8.302
(1.2); 8.297 (1.7); 8.292 (1.2); 7.528 (1.6); 7.516 (1.6); 7.508 (1.5); 7.496 (1.5); 7.466 (1.5); 7.457 (1.7); 7.453 (1.5); 7.443
(2.0); 7.434 (0.5); 7.368 (0.9); 7.359 (1.3); 7.355 (1.3); 7.353 (1.2); 7.344 (2.8); 7.336 (1.8); 7.327 (5.2); 7.318 (3.6); 7.312 (3.3);
7.303 (2.6); 7.294 (0.4); 6.521 (4.3); 5.174 (9.7); 4.017 (10.3); 3.331 (29.9); 2.525 (0.5); 2.511 (10.4); 2.507 (21.1); 2.503 (28.1);
2.498 (21.0); 2.177 (16.0)
Compound No. I-1-248: ¹H NMR (400.0 MHz, d₆-DMSO):
11.921 (1.3); 9.698 (1.1); 9.696 (1.1); 9.693 (0.9); 9.483 (0.9); 9.481 (0.9); 9.470 (0.9); 9.467 (1.0); 8.211 (0.8); 8.205 (0.8); 8.197
(0.8); 8.191 (0.8); 7.226 (2.1); 6.990 (0.4); 6.844 (1.7); 5.057 (0.3); 3.338 (421.5); 3.264 (0.4); 3.249 (1.0); 2.668 (6.5); 2.560
(0.3); 2.542 (32.4); 2.507 (87.9); 2.502 (117.2); 2.498 (88.7); 2.474 (16.0); 2.443 (1.0); 2.410 (1.5); 2.390 (2.9); 2.334 (0.6); 2.329
(0.8); 2.325 (0.6); 1.235 (0.7); 0.000 (0.9)
Compound No. I-1-249: ¹H NMR (400.0 MHz, d₆-DMSO):
13.185 (2.1); 9.661 (2.0); 9.658 (2.5); 9.655 (2.5); 9.652 (2.2); 9.571 (1.7); 9.568 (1.8); 9.558 (1.8); 9.555 (1.9); 8.647 (1.5); 8.637
(1.5); 8.635 (1.5); 8.134 (1.8); 8.128 (1.9); 8.121 (1.9); 8.115 (1.8); 8.093 (0.8); 8.089 (0.8); 8.072 (1.5); 8.068 (1.4); 8.054
(1.2); 8.049 (1.2); 7.986 (2.5); 7.965 (1.6); 7.412 (1.2); 7.399 (1.4); 7.396 (1.3); 7.383 (1.1); 7.381 (1.1); 6.801 (5.0); 3.449 (0.4);
3.333 (745.9); 2.676 (1.5); 2.671 (2.0); 2.667 (1.6); 2.541 (5.8); 2.511 (120.4); 2.507 (232.3); 2.502 (306.6); 2.498 (235.3); 2.333
(1.5); 2.329 (2.0); 2.324 (1.6); 2.295 (16.0); 2.074 (0.9); 1.258 (0.4); 1.234 (1.4); 0.000 (36.1); −0.008 (2.3)
Compound No. I-1-250: ¹H NMR (400.0 MHz, d₆-DMSO):
11.512 (3.1); 9.641 (2.4); 9.638 (2.9); 9.635 (2.9); 9.632 (2.4); 9.457 (2.5); 9.454 (2.5); 9.443 (2.6); 9.441 (2.5); 8.145 (2.2); 8.139
(2.2); 8.132 (2.2); 8.126 (2.1); 6.615 (4.8); 5.054 (1.1); 5.031 (3.5); 5.008 (3.7); 4.985 (1.3); 3.336 (117.8); 3.250 (1.3); 3.110
(2.4); 2.676 (0.7); 2.672 (0.6); 2.542 (32.6); 2.507 (51.3); 2.503 (66.5); 2.498 (49.6); 2.330 (16.0); 1.235 (0.3); 0.000 (7.2)
Compound No. I-1-251: ¹H NMR (400.0 MHz, d₆-DMSO):
11.360 (1.4); 9.630 (0.9); 9.627 (1.1); 9.624 (1.1); 9.621 (1.0); 9.439 (0.9); 9.436 (1.0); 9.425 (1.0); 9.423 (1.0); 8.123 (0.9); 8.118
(0.9); 8.110 (0.9); 8.104 (0.9); 7.173 (2.0); 6.532 (2.0); 5.312 (3.9); 3.337 (12.8); 3.251 (0.4); 3.122 (0.9); 2.543 (12.7); 2.512
(4.3); 2.508 (9.0); 2.503 (12.2); 2.499 (9.2); 2.494 (4.8); 2.373 (16.0); 2.344 (6.4); 0.000 (2.6)
Compound No. I-1-252: ¹H NMR (400.0 MHz, d₆-DMSO):
11.352 (1.2); 9.632 (0.8); 9.629 (1.0); 9.627 (1.0); 9.624 (0.9); 9.441 (0.8); 9.438 (0.9); 9.428 (0.9); 9.425 (0.9); 8.126 (0.8); 8.120
(0.8); 8.112 (0.8); 8.107 (0.8); 6.550 (1.7); 6.160 (2.8); 5.244 (3.3); 3.814 (16.0); 3.342 (41.5); 2.543 (20.1); 2.521 (0.4); 2.512
(4.5); 2.508 (9.4); 2.504 (12.8); 2.499 (9.6); 2.495 (5.0); 2.378 (5.3)
Compound No. I-1-253: ¹H NMR (400.0 MHz, d₆-DMSO):
11.354 (2.6); 9.629 (1.7); 9.626 (2.2); 9.623 (2.2); 9.620 (2.0); 9.439 (1.8); 9.436 (1.9); 9.425 (1.9); 9.422 (2.0); 8.607 (3.1); 8.594
(3.2); 8.122 (1.7); 8.116 (1.8); 8.109 (1.8); 8.103 (1.7); 7.314 (2.4); 7.302 (2.3); 6.537 (3.8); 5.374 (7.5); 3.349 (58.8); 3.116
(1.1); 2.544 (10.2); 2.527 (0.3); 2.513 (6.7); 2.509 (13.9); 2.504 (18.9); 2.500 (14.3); 2.495 (7.4); 2.444 (16.0); 2.331 (12.1)
Compound No. I-1-254: ¹H NMR (400.0 MHz, d₆-DMSO):
11.335 (4.0); 9.613 (3.9); 9.610 (4.0); 9.608 (3.4); 9.427 (3.1); 9.424 (2.0); 9.413 (3.2); 9.411 (1.9); 8.479 (3.0); 8.475 (2.1); 8.467
(3.1); 8.464 (1.9); 8.112 (1.9); 8.109 (2.5); 8.103 (2.2); 8.098 (2.1); 8.096 (2.6); 8.090 (1.9); 7.979 (2.9); 7.975 (1.8); 7.961
(2.4); 7.958 (3.2); 7.955 (1.8); 7.417 (1.7); 7.414 (1.7); 7.405 (1.8); 7.402 (1.9); 7.396 (1.8); 7.394 (1.7); 7.385 (1.7); 7.382 (1.6);
6.539 (5.4); 5.460 (10.2); 5.383 (0.5); 3.345 (157.6); 3.310 (0.5); 3.253 (0.5); 3.250 (0.4); 2.546 (32.5); 2.543 (33.8); 2.529 (1.1);
2.507 (37.0); 2.503 (36.8); 2.351 (16.0); 2.280 (0.7); 0.003 (0.8); 0.000 (0.9)
Compound No. I-1-255: ¹H NMR (400.0 MHz, d₆-DMSO):
11.473 (2.6); 9.613 (2.2); 9.444 (1.8); 9.431 (1.9); 8.127 (2.1); 8.119 (1.8); 8.113 (1.6); 8.106 (1.5); 8.100 (1.5); 6.796 (2.3); 6.790
(2.5); 6.569 (3.5); 6.430 (7.1); 3.337 (776.7); 2.671 (1.5); 2.541 (4.0); 2.506 (169.9); 2.502 (226.5); 2.498 (177.7); 2.479 (16.0);
2.329 (1.4); 2.074 (0.7); 1.235 (1.4); 0.000 (8.4)
Compound No. I-1-256: ¹H NMR (400.0 MHz, d₆-DMSO):
11.428 (3.2); 9.618 (2.8); 9.615 (2.7); 9.446 (2.2); 9.444 (2.2); 9.433 (2.4); 9.431 (2.2); 8.121 (1.9); 8.115 (2.0); 8.108 (2.0); 8.102
(2.0); 7.604 (4.2); 7.310 (4.9); 6.513 (4.5); 6.203 (9.7); 3.339 (569.5); 3.249 (0.4); 3.100 (1.0); 2.676 (1.0); 2.671 (1.3); 2.667
(1.0); 2.542 (25.8); 2.507 (143.0); 2.502 (185.9); 2.498 (142.6); 2.462 (16.0); 2.333 (1.0); 2.329 (1.2); 2.325 (1.0); 2.074 (0.5);
1.989 (15.9); 1.235 (1.1); 0.000 (7.9)
Compound No. I-1-257: ¹H NMR (400.0 MHz, d₆-DMSO):
9.116 (0.4); 9.082 (2.5); 9.077 (2.7); 8.775 (2.2); 8.763 (2.3); 8.316 (0.4); 8.307 (1.6); 8.273 (1.5); 8.253 (1.6); 7.588 (1.4); 7.576
(1.6); 7.567 (1.7); 7.556 (1.6); 5.756 (1.1); 3.371 (0.7); 3.337 (0.7); 3.267 (0.5); 3.169 (3.0); 3.072 (1.3); 2.621 (0.4); 2.506 (32.6);
2.502 (43.4); 2.451 (16.0); 2.432 (0.9); 2.419 (0.7); 2.396 (15.2); 2.365 (0.4); 2.330 (0.4); 2.315 (0.5); 2.234 (1.1); 2.223
(3.6); 0.008 (1.7); 0.000 (38.4)
Compound No. I-1-258: ¹H NMR (400.0 MHz, d₆-DMSO):
10.311 (4.3); 9.082 (3.9); 9.078 (3.9); 8.794 (2.5); 8.790 (2.8); 8.782 (2.7); 8.778 (2.7); 8.287 (1.5); 8.282 (2.3); 8.278 (1.7); 8.267
(1.7); 8.262 (2.4); 8.258 (1.7); 7.611 (2.0); 7.598 (2.1); 7.591 (2.1); 7.579 (1.9); 6.988 (0.8); 3.635 (0.3); 3.616 (0.4); 3.399
(3.7); 3.186 (0.3); 2.891 (1.8); 2.767 (2.1); 2.750 (4.9); 2.733 (6.0); 2.717 (2.2); 2.507 (28.2); 2.503 (37.2); 2.499 (29.1); 2.398
(16.0); 2.244 (1.4); 1.478 (0.5); 1.459 (1.0); 1.453 (0.6); 1.441 (1.2); 1.434 (2.3); 1.416 (4.6); 1.398 (4.6); 1.380 (2.4); 1.362 (0.6);
0.875 (1.4); 0.856 (2.9); 0.836 (7.3); 0.817 (13.4); 0.798 (6.1); 0.007 (2.3); 0.000 (42.1); −0.025 (0.4)
Compound No. I-1-259: ¹H NMR (400.0 MHz, d₆-DMSO):
11.622 (12.5); 9.149 (10.9); 9.144 (10.6); 9.111 (0.4); 9.106 (0.4); 8.771 (1.3); 8.763 (8.1); 8.759 (8.3); 8.751 (7.9); 8.747 (7.3);
8.374 (4.8); 8.369 (6.5); 8.364 (4.7); 8.354 (5.1); 8.349 (6.6); 8.344 (4.2); 8.317 (0.4); 7.569 (0.7); 7.558 (6.2); 7.546 (6.0); 7.538
(6.0); 7.526 (5.3); 6.942 (15.1); 6.666 (0.5); 5.114 (5.1); 5.105 (2.3); 5.092 (15.5); 5.083 (2.7); 5.070 (16.0); 5.047 (5.5); 3.340
(8.7); 3.326 (86.2); 3.177 (0.4); 3.164 (0.3); 2.677 (0.6); 2.673 (0.8); 2.553 (7.4); 2.542 (51.5); 2.512 (51.7); 2.508 (83.6); 2.504
(100.8); 2.499 (72.1); 2.376 (0.3); 2.335 (0.6); 2.330 (0.7); 2.326 (0.5); 2.256 (1.4); 2.236 (1.8); 0.146 (0.5); 0.013 (9.2); 0.008
(8.7); 0.000 (100.6); −0.008 (4.3); −0.150 (0.5)
Compound No. I-1-260: ¹H NMR (400.0 MHz, d₆-DMSO):
10.978 (2.7); 9.102 (2.8); 9.098 (2.8); 8.727 (1.8); 8.723 (2.2); 8.715 (2.2); 8.711 (2.3); 8.319 (1.2); 8.314 (1.8); 8.309 (1.4); 8.299
(1.3); 8.294 (1.8); 8.289 (1.3); 7.530 (1.6); 7.518 (1.7); 7.510 (1.7); 7.498 (1.6); 6.502 (3.9); 6.463 (0.4); 6.399 (0.4); 5.756
(16.0); 5.343 (9.5); 5.264 (1.0); 5.255 (0.4); 5.163 (0.4); 4.949 (0.5); 3.625 (0.4); 3.607 (0.4); 3.325 (17.0); 2.671 (0.6); 2.667 (0.3);
2.541 (0.4); 2.524 (0.7); 2.511 (22.3); 2.507 (46.8); 2.502 (63.7); 2.497 (48.0); 2.493 (24.7); 2.333 (0.4); 2.329 (0.5); 2.324
(0.4); 2.295 (0.5); 2.235 (0.3); 2.158 (14.5); 2.114 (0.6); 2.098 (0.6); 2.087 (0.3); 2.071 (1.0); 2.061 (0.3); 2.050 (1.0); 2.039 (0.5);
2.013 (1.4); 1.951 (0.4); 1.797 (1.5); 1.782 (3.6); 1.774 (3.9); 1.762 (2.3); 1.596 (1.9); 1.584 (3.7); 1.575 (3.9); 1.561 (1.5); 1.551
(0.3); 1.368 (0.4); 1.360 (0.4); 1.314 (0.5); 1.307 (0.3); 1.278 (0.4); 1.214 (0.5); 1.196 (0.8); 1.178 (0.4); 1.156 (0.4); 1.128
(0.3); 1.121 (0.4); 0.932 (0.4); 0.921 (0.3); 0.912 (0.3); 0.888 (0.4); 0.146 (0.4); 0.008 (2.6); 0.000 (78.1); −0.008 (3.7); −0.150
(0.4)
Compound No. I-1-261: ¹H NMR(601.6 MHz, d₆-DMSO):
11.470 (3.4); 9.130 (3.3); 9.127 (3.3); 8.764 (2.3); 8.761 (2.4); 8.756 (2.4); 8.753 (2.4); 8.347 (1.3); 8.344 (2.0); 8.341 (1.4); 8.334
(1.4); 8.331 (2.0); 8.327 (1.4); 7.558 (1.8); 7.550 (1.7); 7.546 (1.8); 7.545 (1.7); 7.538 (1.7); 7.537 (1.7); 6.884 (4.3); 6.883
(4.3); 4.777 (13.9); 3.412 (27.6); 3.339 (18.3); 2.571 (16.0); 2.570 (16.0); 2.508 (10.2); 2.505 (13.8); 2.502 (10.3); 2.237 (0.4)
Compound No. I-1-262: ¹H NMR (400.0 MHz, d₆-DMSO):
10.927 (0.4); 9.108 (0.7); 9.103 (0.7); 8.793 (0.5); 8.781 (0.5); 8.733 (8.4); 8.720 (9.0); 8.710 (0.9); 8.519 (1.9); 8.515 (2.4); 8.510
(3.2); 8.507 (4.3); 8.504 (4.2); 8.318 (0.6); 8.305 (0.4); 8.300 (0.5); 8.295 (0.4); 7.700 (1.0); 7.695 (1.5); 7.690 (1.1); 7.680
(1.1); 7.675 (1.7); 7.670 (1.2); 7.532 (0.4); 7.519 (0.4); 7.512 (0.4); 7.500 (0.4); 7.429 (2.0); 7.417 (3.7); 7.405 (1.9); 7.288 (1.4);
7.276 (1.4); 7.270 (1.3); 7.257 (1.2); 6.420 (0.4); 5.988 (3.1); 5.426 (0.5); 5.370 (9.7); 4.809 (12.0); 3.331 (203.8); 3.175 (0.9);
3.162 (0.8); 2.677 (0.4); 2.672 (0.6); 2.668 (0.5); 2.542 (0.3); 2.526 (1.2); 2.521 (2.0); 2.512 (31.9); 2.508 (67.3); 2.503 (92.0);
2.499 (69.9); 2.494 (36.3); 2.334 (0.5); 2.330 (0.7); 2.325 (0.6); 2.321 (1.1); 2.235 (3.3); 2.201 (16.0); 1.235 (0.5); 0.146 (0.4);
0.008 (2.7); 0.000 (82.5); −0.008 (4.0); −0.150 (0.4)
Compound No. I-1-263: ¹H NMR (400.0 MHz, d₆-DMSO):
11.423 (4.6); 9.145 (3.4); 9.140 (3.6); 8.775 (2.4); 8.771 (2.7); 8.763 (2.6); 8.759 (2.7); 8.367 (1.3); 8.362 (2.0); 8.357 (1.5); 8.347
(1.5); 8.342 (2.2); 8.337 (1.5); 8.114 (4.6); 8.096 (5.5); 8.093 (4.2); 7.827 (0.9); 7.813 (0.7); 7.808 (2.8); 7.790 (2.2); 7.737
(3.8); 7.717 (5.4); 7.699 (2.2); 7.574 (1.8); 7.562 (1.8); 7.555 (1.8); 7.542 (1.7); 6.848 (4.8); 6.846 (5.0); 3.384 (23.4); 3.327 (28.6);
3.176 (0.9); 3.163 (0.9); 2.672 (0.3); 2.525 (0.8); 2.512 (17.9); 2.507 (36.8); 2.503 (49.6); 2.498 (38.2); 2.494 (20.3); 2.405
(16.0); 2.366 (0.3); 2.329 (0.3); 0.008 (1.4); 0.000 (37.5); −0.008 (2.0)
Compound No. I-1-264: ¹H NMR (400.0 MHz, d₆-DMSO):
8.793 (0.4); 8.780 (0.4); 8.733 (8.2); 8.721 (8.4); 8.462 (3.2); 8.456 (3.2); 7.631 (1.0); 7.612 (1.0); 7.425 (2.1); 7.413 (4.0); 7.400
(2.0); 7.241 (0.9); 7.229 (1.1); 7.223 (1.0); 7.210 (0.8); 5.961 (1.0); 5.757 (9.3); 5.427 (0.5); 5.340 (8.9); 4.518 (6.3); 4.512 (6.6);
3.328 (24.1); 3.184 (1.9); 3.178 (4.4); 3.172 (2.0); 2.891 (0.7); 2.732 (0.6); 2.525 (0.5); 2.511 (11.8); 2.507 (24.4); 2.503 (33.0);
2.498 (25.3); 2.494 (13.3); 2.321 (0.8); 2.197 (16.0); 1.989 (0.8); 1.175 (0.4); 0.008 (0.9); 0.000 (24.8); −0.008 (1.3)
Compound No. I-1-265: ¹H NMR (400.0 MHz, d₆-DMSO):
8.780 (0.3); 8.735 (7.8); 8.723 (8.0); 8.478 (1.6); 8.474 (1.8); 8.462 (3.7); 8.456 (2.8); 7.627 (1.2); 7.607 (1.4); 7.427 (1.9); 7.415
(3.7); 7.402 (1.9); 7.252 (1.1); 7.240 (1.2); 7.233 (1.2); 7.221 (1.0); 6.417 (0.4); 6.406 (0.8); 6.396 (0.4); 6.277 (0.7); 6.267 (1.7);
6.256 (0.8); 6.138 (0.4); 6.127 (0.8); 6.116 (0.4); 5.905 (1.4); 5.756 (0.5); 5.425 (0.4); 5.337 (9.4); 4.182 (1.1); 4.172 (1.3); 4.146
(2.5); 4.136 (2.5); 4.110 (1.3); 4.100 (1.2); 3.323 (9.1); 2.891 (0.9); 2.731 (0.8); 2.671 (0.4); 2.541 (1.3); 2.524 (0.8); 2.510
(23.7); 2.506 (48.8); 2.502 (66.2); 2.497 (50.2); 2.333 (0.4); 2.328 (0.5); 2.320 (0.8); 2.299 (0.4); 2.176 (16.0); 0.146 (0.4); 0.008
(3.2); 0.000 (86.2); −0.008 (3.9); −0.150 (0.4)
Compound No. I-1-266: ¹H NMR (600.1 MHz, d₆-DMSO):
11.411 (1.9); 9.138 (1.6); 9.135 (1.7); 8.766 (1.2); 8.763 (1.3); 8.758 (1.3); 8.755 (1.2); 8.354 (0.7); 8.351 (1.0); 8.348 (0.7); 8.341
(0.7); 8.338 (1.0); 8.334 (0.7); 7.561 (0.9); 7.560 (0.9); 7.553 (0.9); 7.552 (0.9); 7.547 (0.9); 7.546 (0.9); 7.539 (0.9); 7.538
(0.8); 6.872 (2.2); 6.871 (2.2); 3.389 (1.7); 3.377 (3.5); 3.365 (1.9); 3.326 (5.0); 2.836 (1.9); 2.824 (3.8); 2.811 (1.8); 2.553 (8.2);
2.552 (7.9); 2.510 (2.4); 2.508 (5.1); 2.505 (7.0); 2.502 (5.1); 2.499 (2.5); 2.103 (16.0)
Compound No. I-1-267: ¹H NMR (400.0 MHz, d₆-DMSO):
10.642 (1.8); 9.114 (0.8); 8.817 (6.9); 8.804 (7.1); 8.786 (0.7); 8.759 (0.8); 8.315 (1.3); 8.295 (1.1); 8.171 (0.8); 8.063 (2.8); 8.052
(2.8); 7.563 (1.1); 7.551 (1.2); 7.543 (1.1); 7.531 (0.9); 7.487 (0.4); 7.481 (1.8); 7.475 (0.7); 7.469 (3.3); 7.457 (1.6); 5.527
(1.0); 5.455 (8.1); 5.284 (4.8); 3.427 (0.3); 3.406 (0.5); 3.382 (2.1); 3.365 (3.2); 3.345 (2.8); 3.319 (0.7); 3.169 (3.0); 3.111 (2.0);
3.092 (2.4); 3.074 (1.6); 2.671 (0.4); 2.667 (0.3); 2.525 (1.0); 2.511 (27.6); 2.507 (57.8); 2.502 (77.9); 2.498 (58.6); 2.494 (30.3);
2.333 (0.4); 2.329 (0.7); 2.325 (0.4); 2.075 (16.0); 0.907 (0.5); 0.888 (1.0); 0.870 (0.5); 0.146 (0.4); 0.008 (3.3); 0.000 (103.3);
−0.009 (5.0); −0.150 (0.5)
Compound No. I-1-268: ¹H NMR (400.0 MHz, d₆-DMSO):
11.473 (3.0); 9.148 (0.7); 8.768 (0.7); 8.366 (0.8); 8.361 (1.3); 8.357 (0.9); 8.346 (0.9); 8.341 (1.4); 8.337 (0.9); 7.570 (0.9); 7.558
(1.0); 7.550 (1.0); 7.538 (0.9); 6.904 (3.3); 6.902 (3.4); 3.637 (0.6); 3.603 (0.7); 3.597 (0.8); 3.588 (1.0); 3.581 (2.4); 3.577
(1.9); 3.562 (2.9); 3.534 (2.7); 3.518 (2.2); 3.510 (1.0); 3.501 (0.7); 3.495 (0.6); 3.325 (12.2); 3.063 (16.0); 3.010 (0.5); 2.557 (11.6);
2.525 (0.5); 2.511 (10.3); 2.507 (20.9); 2.503 (27.9); 2.498 (20.9); 2.494 (10.6); 0.000 (3.4)
Compound No. I-1-269: ¹H NMR (600.1 MHz, d₆-DMSO):
11.510 (4.0); 9.122 (4.1); 9.119 (4.2); 8.837 (13.5); 8.829 (13.7); 8.753 (3.1); 8.750 (3.2); 8.745 (3.3); 8.742 (3.2); 8.335 (1.7);
8.332 (2.5); 8.329 (1.7); 8.322 (1.9); 8.319 (2.5); 8.316 (1.7); 7.550 (2.3); 7.549 (2.2); 7.542 (2.3); 7.541 (2.2); 7.536 (2.3); 7.535
(2.2); 7.528 (2.2); 7.527 (2.1); 7.512 (2.9); 7.504 (5.7); 7.496 (2.8); 7.476 (12.5); 5.757 (1.2); 5.721 (16.0); 3.325 (68.2); 3.174
(1.3); 3.165 (1.3); 2.524 (0.4); 2.520 (0.5); 2.517 (0.5); 2.509 (15.6); 2.506 (33.7); 2.503 (46.4); 2.500 (33.5); 2.497 (15.5)
Compound No. I-1-270: ¹H NMR (400.0 MHz, d₆-DMSO):
11.252 (2.0); 9.137 (1.9); 9.132 (1.9); 8.803 (1.3); 8.799 (1.4); 8.791 (1.4); 8.787 (1.4); 8.347 (0.8); 8.342 (1.2); 8.337 (0.8); 8.327
(0.8); 8.322 (1.2); 8.317 (0.8); 7.954 (1.4); 7.600 (1.0); 7.587 (1.1); 7.580 (1.0); 7.568 (1.0); 5.757 (0.7); 3.903 (14.0); 3.327
(4.8); 2.892 (10.4); 2.732 (8.8); 2.580 (16.0); 2.512 (5.0); 2.508 (9.6); 2.503 (12.6); 2.499 (9.4); 0.000 (1.1)
Compound No. I-1-271: mass (m/z): 295.1 (M + H)⁺; ¹H NMR (DMSO-d6) 3.75 (s, 3H), 5.67 (d, 1H), 6.95 (m,
2H), 7.56 (m, 4H), 8.00 (d, 1H), 8.36 (d, 1H), 11.35 (s, 1H).
Compound No. I-1-272: mass (m/z): 351.1 (M + H)⁺; ¹H NMR (DMSO-d6) 1.24 (t, 3H), 4.16 (q, 2H), 5.34 (s, 2H),
7.1-7.4 (m. 5H). 7.49 (s, 1H), 7.55 (m, 1H), 8.26 (m, 1H), 8.75 (m, 1H), 9.07 (m, 1H), 10.48 (s, 1H).
Compound No. I-1-273: mass (m/z): 272.1 (M + H)⁺; ¹H NMR (DMSO-d6) 7.06 (d, 1H), 7.55 (d, 1H), 7.57 (m,
1H), 7.64 (d, 1H), 8.37 (m, 1H), 8.49 (d, 1H), 8.76 (m, 1H), 9.17 (m, 1H), 11.56 (s, 1H).
Compound No. I-1-274: mass (m/z): 299.1/301.0 (M + H)⁺; ¹H NMR (DMSO-d6) 7.02 (s, 1H), 7.45-7.55 (m,
6H), 8.35 (m, 1H), 8.76 (m, 1H), 9.15 (m, 1H), 11.49 (s, 1H).
Compound No. I-1-275: mass (m/z): 303.0/305.0 (M + H)⁺; ¹H NMR (DMSO-d6) 3.73 (s, 3H), 7.37 (t, 1H),
7.58 (m, 1H), 8.30 (m, 1H), 8.79 (m, 1H), 9.11 (1H), 10.61 (s, 1H).
Compound No. I-1-276: ¹H NMR (400.0 MHz, d₆-DMSO):
11.105 (1.0); 9.037 (1.2); 9.032 (1.1); 8.793 (2.7); 8.785 (1.5); 8.780 (3.5); 8.424 (0.8); 8.419 (1.1); 8.414 (0.6); 7.954 (2.3); 7.455
(0.6); 7.443 (1.2); 7.430 (0.6); 6.525 (1.5); 5.430 (3.2); 3.330 (19.2); 2.892 (16.0); 2.733 (14.4); 2.512 (4.5); 2.508 (7.9); 2.504
(9.5); 2.499 (6.5); 2.323 (5.4); 0.000 (2.5)
Compound No. I-1-277: ¹H NMR(601.6 MHz, DMF):
11.525 (8.1); 10.468 (0.4); 9.332 (10.9); 9.331 (11.5); 9.328 (11.7); 9.327 (10.6); 9.155 (1.3); 9.154 (1.4); 9.152 (1.4); 9.151 (1.3);
8.862 (1.0); 8.860 (1.1); 8.855 (1.1); 8.852 (1.0); 8.828 (8.3); 8.826 (8.8); 8.820 (8.7); 8.818 (8.6); 8.531 (5.3); 8.528 (7.1);
8.524 (5.2); 8.517 (5.5); 8.515 (7.2); 8.514 (7.2); 8.511 (5.3); 8.340 (0.6); 8.337 (0.9); 8.336 (0.8); 8.333 (0.7); 8.327 (0.7); 8.324
(0.9); 8.323 (0.9); 8.320 (0.7); 8.027 (8.9); 7.671 (0.8); 7.670 (0.8); 7.663 (0.8); 7.662 (0.8); 7.658 (0.8); 7.657 (0.8); 7.650 (0.7);
7.649 (0.7); 7.627 (6.3); 7.626 (6.2); 7.619 (6.3); 7.618 (6.3); 7.614 (6.4); 7.612 (6.2); 7.606 (6.1); 7.604 (5.9); 6.925 (16.0);
6.923 (15.8); 6.659 (3.3); 5.817 (0.8); 4.071 (0.8); 4.059 (0.8); 3.669 (0.5); 3.622 (12.5); 3.555 (97.7); 3.513 (0.7); 3.487 (49.6);
3.436 (0.5); 3.232 (0.6); 2.953 (7.6); 2.931 (0.4); 2.922 (4.1); 2.919 (8.3); 2.916 (11.4); 2.913 (8.1); 2.910 (4.0); 2.785 (7.3); 2.752
(4.5); 2.749 (8.9); 2.745 (12.5); 2.742 (8.6); 2.739 (4.4); 2.674 (0.4); 2.673 (0.4); 2.568 (63.2); 2.566 (62.3); 2.505 (0.4); 2.504
(0.4); 2.457 (0.4); 2.456 (0.4); 2.295 (11.3); 2.014 (3.7); 1.418 (0.3); 1.380 (0.6); 1.297 (0.9); 1.278 (0.9); 1.211 (1.1); 1.200
(2.0); 1.188 (1.1); 0.005 (0.4); 0.000 (9.9); −0.006 (0.3)
Compound No. I-1-278: ¹H NMR (400.0 MHz, d₆-DMSO):
11.587 (5.0); 9.146 (4.3); 9.141 (4.3); 8.764 (2.9); 8.760 (3.1); 8.752 (3.1); 8.748 (3.0); 8.367 (1.8); 8.363 (2.6); 8.357 (1.8); 8.347
(2.0); 8.343 (2.7); 8.337 (1.8); 7.560 (2.4); 7.548 (2.4); 7.540 (2.3); 7.528 (2.2); 6.887 (6.5); 4.039 (1.0); 4.021 (1.0); 4.003
(0.3); 3.656 (2.2); 3.637 (7.0); 3.619 (7.1); 3.601 (2.3); 3.329 (14.3); 2.509 (36.2); 2.505 (24.0); 2.500 (16.5); 2.264 (0.3); 1.990
(4.2); 1.194 (1.1); 1.176 (2.3); 1.159 (8.3); 1.141 (16.0); 1.122 (7.2); 0.008 (1.8); 0.000 (30.7); −0.008 (1.2)
Compound No. I-1-279: ¹H NMR (600.1 MHz, d₆-DMSO):
3.502 (16.0); 2.667 (0.4); 2.666 (0.4)
Compound No. I-1-280: ¹H NMR (400.0 MHz, d₆-DMSO):
11.578 (3.9); 10.750 (0.3); 9.129 (3.3); 9.124 (3.2); 9.096 (3.5); 9.091 (3.6); 8.958 (2.4); 8.954 (2.5); 8.946 (2.5); 8.942 (2.5); 8.748
(2.2); 8.744 (2.4); 8.736 (2.4); 8.732 (2.3); 8.373 (1.4); 8.368 (1.9); 8.363 (1.6); 8.353 (1.6); 8.347 (2.0); 8.343 (1.6); 8.320
(1.4); 8.315 (2.1); 8.310 (1.5); 8.300 (1.5); 8.295 (2.1); 8.290 (1.4); 7.748 (1.8); 7.736 (1.9); 7.729 (1.9); 7.717 (1.8); 7.536 (1.8);
7.524 (1.8); 7.517 (1.7); 7.504 (1.6); 6.908 (4.8); 6.907 (4.8); 6.524 (0.6); 3.332 (17.0); 2.611 (16.0); 2.510 (18.4); 2.506 (23.3);
2.502 (17.3); 2.177 (1.7); 1.991 (1.3); 1.194 (0.3); 1.176 (0.7); 1.158 (0.3); 0.000 (29.0)
Compound No. I-1-281: ¹H NMR (400.0 MHz, d₆-DMSO):
11.079 (0.9); 9.098 (0.8); 8.724 (0.5); 8.712 (0.5); 8.316 (0.4); 8.312 (0.5); 8.296 (0.4); 8.292 (0.5); 7.525 (0.5); 7.513 (0.5); 7.506
(0.4); 7.493 (0.4); 6.563 (1.2); 5.535 (2.8); 3.327 (8.6); 2.507 (10.6); 2.503 (12.3); 2.365 (4.4); 1.338 (1.7); 1.324 (16.0); 1.296
(0.4); 1.253 (0.6); 1.152 (0.6); 1.124 (0.3); 1.099 (0.9); 0.000 (7.6)
Compound No. I-1-282: ¹H NMR (400.0 MHz, d₆-DMSO):
11.046 (2.1); 9.098 (2.1); 9.094 (2.1); 8.722 (1.6); 8.718 (1.5); 8.710 (1.6); 8.706 (1.4); 8.317 (0.9); 8.312 (1.2); 8.307 (0.8); 8.297
(0.9); 8.292 (1.2); 8.287 (0.8); 7.523 (1.1); 7.511 (1.1); 7.504 (1.1); 7.492 (1.0); 6.530 (3.2); 5.358 (7.5); 5.234 (0.4); 5.025
(0.4); 3.334 (13.6); 2.572 (16.0); 2.543 (7.9); 2.508 (11.9); 2.503 (14.5); 2.499 (10.3); 2.362 (11.3); 2.220 (0.5); 0.000 (5.8)
Compound No. I-1-283: ¹H NMR (400.0 MHz, d₆-DMSO):
11.054 (2.6); 9.104 (2.5); 9.100 (2.5); 8.724 (1.9); 8.720 (2.0); 8.712 (2.0); 8.708 (1.9); 8.322 (1.0); 8.318 (1.5); 8.313 (1.0); 8.302
(1.1); 8.298 (1.5); 8.293 (1.0); 7.526 (1.5); 7.514 (1.5); 7.506 (1.5); 7.494 (1.4); 6.524 (3.9); 6.042 (3.9); 5.757 (2.1); 5.251
(10.5); 3.329 (17.7); 2.525 (0.6); 2.507 (19.0); 2.503 (24.8); 2.498 (17.9); 2.411 (0.4); 2.405 (0.5); 2.388 (0.4); 2.366 (16.0); 2.344
(1.7); 2.329 (0.9); 2.314 (15.5); 2.283 (0.4); 2.274 (0.5); 2.216 (0.4); 2.144 (0.8); 0.000 (0.5)
Compound No. I-1-284: ¹H NMR (600.1 MHz, d₆-DMSO):
11.061 (2.2); 9.107 (2.1); 9.104 (2.1); 8.727 (1.5); 8.725 (1.6); 8.719 (1.5); 8.717 (1.5); 8.319 (0.8); 8.317 (1.2); 8.314 (0.9); 8.306
(0.9); 8.303 (1.2); 8.300 (0.8); 7.524 (1.1); 7.516 (1.2); 7.511 (1.2); 7.503 (1.1); 6.575 (3.0); 5.759 (1.2); 5.628 (8.0); 3.337
(5.9); 2.509 (3.6); 2.506 (4.7); 2.503 (3.6); 2.350 (11.9); 2.337 (16.0)
Compound No. I-1-285: ¹H NMR (400.0 MHz, d₆-DMSO):
11.062 (1.0); 9.096 (1.0); 8.720 (0.7); 8.708 (0.7); 8.314 (0.5); 8.294 (0.5); 7.523 (0.5); 7.510 (0.5); 7.503 (0.5); 7.491 (0.4); 6.538
(1.4); 5.377 (2.9); 3.325 (4.8); 2.503 (10.6); 2.501 (10.1); 2.362 (4.6); 2.145 (0.3); 1.362 (16.0); 1.305 (1.2); 1.069 (0.5); 1.068
(0.5)
Compound No. I-1-286: ¹H NMR (600.1 MHz, CD3CN):
9.325 (0.7); 9.045 (1.3); 9.042 (1.3); 8.696 (1.0); 8.694 (1.0); 8.688 (1.0); 8.686 (1.0); 8.200 (0.5); 8.197 (0.8); 8.194 (0.5); 8.187
(0.6); 8.184 (0.8); 8.180 (0.5); 7.437 (0.7); 7.436 (0.7); 7.429 (0.8); 7.428 (0.7); 7.424 (0.8); 7.423 (0.7); 7.416 (0.7); 7.415 (0.7);
6.583 (1.6); 5.547 (0.5); 5.364 (8.1); 3.140 (0.8); 3.098 (16.0); 3.063 (0.8); 3.039 (15.1); 2.508 (7.9); 2.3553 (9.4); 2.3545 (9.0);
2.269 (0.4); 2.256 (0.5); 2.246 (0.3); 1.951 (0.9); 1.947 (1.5); 1.943 (2.2); 1.939 (1.5); 1.935 (0.8)
Compound No. I-1-287: mass (m/z): 288.1 (M + H)⁺; ¹H NMR (DMSO-d6) 1.00 (dd, 6 H), 2.53 (s, 3H), 3.63 (m,
1H), 5.48 (d, 1H), 6.72 (s, 1H), 7.56 (dd, 1H), 8.76 (m, 1H), 9.13 (m, 1H), 11.25 (s, 1H).
Compound No. I-1-288: ¹H NMR (400.0 MHz, d₆-DMSO):
11.189 (2.0); 9.121 (4.4); 9.117 (4.3); 8.765 (3.0); 8.761 (3.2); 8.753 (3.1); 8.749 (3.0); 8.335 (1.7); 8.330 (2.5); 8.325 (1.7); 8.315
(2.7); 8.310 (2.7); 8.305 (1.7); 7.841 (1.1); 7.826 (2.0); 7.812 (1.1); 7.568 (2.3); 7.556 (2.3); 7.548 (2.2); 7.536 (2.1); 6.698
(6.2); 3.337 (231.3); 3.311 (2.3); 3.293 (4.0); 3.277 (4.5); 3.275 (4.5); 3.260 (3.8); 3.242 (1.2); 2.676 (0.4); 2.672 (0.5); 2.668 (0.4);
2.531 (22.9); 2.507 (57.4); 2.503 (73.7); 2.499 (53.6); 2.334 (0.4); 2.330 (0.5); 2.325 (0.4); 2.075 (1.1); 1.150 (7.6); 1.133 (16.0);
1.115 (7.3); 0.008 (2.5); 0.000 (52.4); −0.008 (2.1)
Compound No. I-1-290: ¹H NMR (400.0 MHz, d₆-DMSO):
10.968 (2.7); 9.032 (2.5); 9.028 (2.5); 8.806 (1.8); 8.802 (1.9); 8.794 (1.9); 8.790 (1.8); 8.230 (1.0); 8.225 (1.4); 8.220 (1.0); 8.210
(1.1); 8.205 (1.5); 8.200 (1.0); 7.622 (1.3); 7.620 (1.3); 7.609 (1.3); 7.608 (1.3); 7.602 (1.3); 7.600 (1.2); 7.590 (1.2); 7.588
(1.2); 6.389 (5.5); 3.445 (1.7); 3.338 (30.2); 2.543 (19.8); 2.513 (6.4); 2.508 (12.6); 2.504 (16.2); 2.499 (11.6); 2.495 (5.6); 2.209
(16.0); 1.195 (4.5); 0.000 (4.4)
Compound No. I-1-291: ¹H NMR (400.0 MHz, d₆-DMSO):
11.203 (1.5); 9.120 (1.2); 8.750 (0.9); 8.741 (0.9); 8.341 (0.6); 8.337 (0.8); 8.332 (0.6); 8.321 (0.6); 8.317 (0.9); 8.312 (0.6); 7.554
(0.8); 7.542 (0.8); 7.535 (0.8); 7.522 (0.7); 6.682 (2.1); 3.329 (7.9); 3.021 (16.0); 2.543 (0.9); 2.533 (0.5); 2.508 (8.8); 2.504
(11.2); 2.499 (8.7); 2.365 (7.7); 0.000 (8.4)
Compound No. I-1-292: ¹H NMR (400.0 MHz, d₆-DMSO):
11.157 (4.6); 9.113 (4.4); 9.109 (4.4); 9.107 (4.1); 8.766 (3.4); 8.761 (3.6); 8.754 (3.6); 8.749 (3.5); 8.326 (1.9); 8.321 (2.6); 8.316
(2.0); 8.306 (2.1); 8.301 (2.8); 8.296 (2.0); 7.774 (1.7); 7.763 (1.6); 7.570 (2.5); 7.568 (2.4); 7.558 (2.4); 7.556 (2.4); 7.550
(2.4); 7.548 (2.3); 7.538 (2.3); 7.536 (2.3); 6.689 (6.7); 6.687 (6.5); 3.334 (21.5); 2.817 (16.0); 2.805 (15.8); 2.531 (23.3); 2.530
(23.3); 2.512 (11.4); 2.508 (22.9); 2.503 (30.0); 2.499 (21.5); 2.494 (10.2); 0.008 (1.8); 0.000 (45.9); −0.009 (1.5)
Compound No. I-1-293: ¹H NMR (600.1 MHz, CD3CN):
9.143 (1.1); 9.044 (2.8); 8.708 (2.1); 8.701 (2.1); 8.197 (1.6); 8.184 (1.6); 7.453 (1.4); 7.445 (1.6); 7.440 (1.5); 7.432 (1.3); 6.949
(3.9); 6.846 (3.6); 6.497 (3.1); 5.211 (9.4); 3.810 (3.0); 3.691 (16.0); 3.276 (8.7); 2.351 (15.7); 2.300 (1.9); 2.271 (1.1); 2.243
(0.4); 1.964 (0.6); 1.956 (1.6); 1.952 (2.0); 1.948 (10.7); 1.944 (18.5); 1.939 (27.1); 1.935 (18.5); 1.931 (9.5); 0.000 (0.4)
Compound No. I-1-294: ¹H NMR (400.0 MHz, d₆-DMSO):
11.239 (3.3); 9.133 (3.0); 9.129 (3.0); 8.789 (2.1); 8.779 (2.1); 8.777 (2.0); 8.746 (2.3); 8.742 (2.4); 8.734 (2.4); 8.729 (2.4); 8.354
(1.3); 8.350 (1.8); 8.345 (1.3); 8.335 (1.4); 8.329 (1.8); 8.325 (1.3); 8.077 (2.3); 8.073 (2.3); 8.057 (2.7); 8.053 (2.5); 7.733
(2.6); 7.721 (2.5); 7.713 (2.4); 7.701 (2.3); 7.550 (1.7); 7.548 (1.7); 7.538 (1.7); 7.536 (1.6); 7.530 (1.6); 7.528 (1.6); 7.518 (1.6)
7.516 (1.6); 6.789 (4.6); 6.687 (4.0); 6.627 (4.0); 3.345 (217.5); 3.176 (0.5); 3.163 (0.5); 2.543 (1.5); 2.526 (0.8); 2.513 (18.7);
2.508 (38.0); 2.504 (50.2); 2.499 (36.4); 2.495 (17.6); 2.331 (0.3); 2.157 (16.0)
Compound No. I-1-295: ¹H NMR (400.0 MHz, d₆-DMSO):
11.021 (2.8); 9.097 (2.7); 9.093 (2.7); 9.074 (4.7); 9.069 (4.5); 8.716 (2.3); 8.712 (2.4); 8.704 (2.4); 8.700 (2.3); 8.315 (1.2); 8.310
(1.6); 8.305 (1.1); 8.295 (1.2); 8.290 (1.6); 8.286 (1.1); 7.518 (1.6); 7.516 (1.7); 7.506 (1.6); 7.504 (1.6); 7.498 (1.6); 7.496
(1.6); 7.486 (1.5); 7.484 (1.5); 7.463 (3.1); 7.458 (3.0); 6.505 (4.2); 5.409 (0.6); 5.336 (9.1); 3.327 (49.1); 2.542 (2.9); 2.525 (0.9);
2.511 (17.0); 2.507 (33.8); 2.502 (44.2); 2.498 (31.9); 2.493 (15.3); 2.3664 (16.0); 2.3656 (15.9); 2.329 (0.4); 2.324 (0.8); 2.282
(0.4); 2.139 (1.2); 0.008 (1.0); 0.000 (25.8); −0.009 (1.0)
Compound No. I-1-296: ¹H NMR (400.0 MHz, d₆-DMSO):
11.105 (2.2); 9.105 (2.1); 9.100 (2.1); 8.728 (1.6); 8.724 (1.7); 8.716 (1.7); 8.712 (1.6); 8.324 (0.8); 8.319 (1.2); 8.314 (0.8); 8.304
(0.9); 8.299 (1.3); 8.295 (0.8); 7.529 (1.2); 7.517 (1.2); 7.509 (1.2); 7.497 (1.2); 6.554 (3.2); 6.185 (0.3); 5.757 (1.3); 5.662
(8.6); 4.195 (0.5); 3.874 (1.1); 3.859 (1.0); 3.329 (16.8); 2.727 (2.7); 2.687 (16.0); 2.668 (1.8); 2.632 (5.3); 2.542 (1.3); 2.507 (20.1);
2.503 (26.1); 2.498 (19.2); 2.335 (12.4); 2.161 (1.3); 0.008 (0.5); 0.000 (13.4); −0.008 (0.6)
Compound No. I-1-297: ¹H NMR (400.0 MHz, d₆-DMSO):
11.074 (2.6); 9.110 (2.6); 9.106 (2.6); 8.727 (2.3); 8.723 (2.5); 8.715 (2.5); 8.711 (2.4); 8.330 (1.1); 8.326 (1.6); 8.321 (1.1); 8.317
(0.4); 8.310 (1.2); 8.305 (1.6); 8.301 (1.1); 7.529 (1.6); 7.527 (1.6); 7.517 (1.6); 7.515 (1.6); 7.509 (1.6); 7.507 (1.6); 7.497
(1.5); 7.495 (1.5); 7.001 (4.2); 6.992 (6.5); 6.959 (3.8); 6.950 (2.5); 6.512 (4.1); 5.353 (8.5); 3.329 (27.3); 2.542 (1.8); 2.526 (0.4);
2.521 (0.7); 2.512 (10.3); 2.508 (21.0); 2.503 (27.7); 2.499 (19.8); 2.494 (9.3); 2.308 (16.0); 2.307 (16.0); 0.008 (0.6); 0.000
(18.9); −0.009 (0.6)
Compound No. I-1-298: ¹H NMR (400.0 MHz, d₆-DMSO):
11.075 (2.0); 9.101 (1.8); 9.096 (1.8); 8.723 (1.3); 8.719 (1.4); 8.711 (1.4); 8.707 (1.4); 8.320 (0.7); 8.315 (1.1); 8.311 (0.7); 8.300
(0.8); 8.295 (1.1); 8.291 (0.7); 7.523 (1.0); 7.511 (1.0); 7.503 (1.0); 7.491 (0.9); 6.639 (3.8); 6.552 (2.8); 5.499 (5.8); 5.230
(0.4); 3.333 (31.0); 3.063 (1.0); 3.049 (16.0); 3.007 (1.0); 2.995 (14.0); 2.542 (25.0); 2.512 (6.8); 2.508 (13.6); 2.503 (17.8); 2.499
(13.0); 2.494 (6.4); 2.369 (10.1); 0.008 (0.4); 0.000 (10.9); −0.008 (0.4)
Compound No. I-1-299: ¹H NMR (400.0 MHz, d₆-DMSO):
11.082 (3.2); 9.092 (2.7); 9.087 (2.7); 8.717 (2.0); 8.713 (2.2); 8.705 (2.1); 8.701 (2.1); 8.310 (1.1); 8.306 (1.7); 8.301 (1.2); 8.291
(1.3); 8.285 (1.8); 8.281 (1.2); 7.981 (0.4); 7.974 (3.1); 7.971 (3.9); 7.967 (1.9); 7.960 (1.2); 7.955 (4.1); 7.950 (3.6); 7.667
(0.5); 7.663 (0.4); 7.658 (0.3); 7.648 (1.8); 7.641 (0.8); 7.635 (1.7); 7.631 (3.0); 7.623 (4.4); 7.619 (2.1); 7.608 (2.0); 7.604 (4.4);
7.592 (0.8); 7.588 (1.6); 7.582 (1.1); 7.570 (0.4); 7.516 (1.6); 7.515 (1.6); 7.503 (1.7); 7.496 (1.6); 7.495 (1.6); 7.484 (1.5); 7.483
(1.6); 6.594 (4.4); 5.758 (0.7); 5.659 (11.0); 4.780 (0.6); 4.104 (0.4); 4.091 (0.4); 3.330 (23.5); 3.177 (2.1); 3.164 (2.1); 2.543
(1.5); 2.526 (0.6); 2.513 (14.4); 2.508 (29.3); 2.504 (38.8); 2.499 (28.3); 2.495 (13.8); 2.432 (16.0); 2.164 (1.0); 0.008 (0.7); 0.000
(21.4); −0.009 (0.8)
Compound No. I-1-300: ¹H NMR (400.0 MHz, d₆-DMSO):
11.088 (3.3); 9.101 (2.9); 9.096 (2.9); 8.722 (2.1); 8.718 (2.3); 8.710 (2.3); 8.706 (2.2); 8.319 (1.1); 8.315 (1.7); 8.310 (1.1); 8.299
(1.2); 8.294 (1.8); 8.290 (1.2); 8.011 (0.4); 8.005 (3.1); 8.001 (3.9); 7.990 (1.3); 7.985 (4.0); 7.981 (3.6); 7.631 (0.5); 7.628
(0.3); 7.613 (1.7); 7.606 (0.8); 7.600 (1.7); 7.596 (3.2); 7.589 (4.5); 7.570 (4.3); 7.558 (0.8); 7.554 (1.3); 7.548 (1.0); 7.523 (1.6);
7.521 (1.6); 7.511 (1.6); 7.509 (1.6); 7.503 (1.6); 7.502 (1.6); 7.491 (1.5); 7.489 (1.5); 6.608 (4.5); 5.754 (10.9); 4.105 (0.5); 4.092
(0.5); 3.331 (40.4); 3.177 (2.1); 3.164 (2.0); 2.543 (0.4); 2.526 (0.6); 2.512 (12.0); 2.508 (23.7); 2.504 (30.9); 2.499 (22.4);
2.495 (10.8); 2.405 (16.0); 0.008 (0.8); 0.000 (20.2); −0.009 (0.7)
Compound No. I-1-301: ¹H NMR (400.0 MHz, d₆-DMSO):
11.064 (3.3); 11.035 (1.2); 9.101 (3.9); 9.098 (4.1); 8.721 (2.4); 8.718 (2.9); 8.710 (2.8); 8.706 (3.0); 8.316 (2.3); 8.296 (2.4); 8.136
(3.5); 8.115 (3.6); 7.866 (1.2); 7.847 (1.2); 7.523 (1.9); 7.510 (2.1); 7.504 (2.1); 7.492 (1.9); 7.040 (3.1); 7.020 (3.0); 6.635
(1.0); 6.615 (1.0); 6.576 (4.4); 6.559 (1.7); 5.337 (9.5); 5.235 (3.0); 3.895 (6.9); 3.328 (73.5); 2.902 (0.6); 2.672 (0.4); 2.542 (19.6);
2.507 (55.2); 2.503 (68.9); 2.499 (54.8); 2.355 (5.4); 2.329 (0.8); 2.309 (16.0); 0.000 (30.3)
Compound No. I-1-302: ¹H NMR (400.0 MHz, d₆-DMSO):
11.055 (2.9); 9.101 (2.7); 9.097 (2.7); 8.721 (2.2); 8.716 (2.3); 8.709 (2.3); 8.704 (2.3); 8.319 (1.1); 8.314 (1.6); 8.309 (1.1); 8.299
(1.2); 8.294 (1.7); 8.289 (1.1); 7.891 (5.2); 7.522 (1.6); 7.520 (1.6); 7.510 (1.6); 7.508 (1.5); 7.502 (1.5); 7.500 (1.5); 7.490
(1.5); 7.488 (1.5); 6.542 (4.4); 5.421 (9.1); 3.333 (23.9); 2.543 (35.1); 2.526 (0.4); 2.521 (0.6); 2.513 (7.3); 2.509 (15.0); 2.504
(19.8); 2.500 (14.3); 2.495 (6.8); 2.363 (16.0); 0.008 (0.5); 0.000 (14.2); −0.009 (0.5)
Compound No. I-1-303: ¹H NMR (400.0 MHz, d₆-DMSO):
11.002 (2.3); 9.087 (2.0); 9.083 (2.0); 8.712 (1.6); 8.708 (1.7); 8.700 (1.7); 8.696 (1.6); 8.306 (0.8); 8.302 (1.2); 8.297 (0.8); 8.286
(0.9); 8.281 (1.3); 8.277 (0.8); 7.938 (2.4); 7.935 (3.1); 7.916 (3.2); 7.914 (3.0); 7.560 (1.9); 7.555 (0.8); 7.541 (3.2); 7.520
(2.3); 7.511 (1.2); 7.510 (1.2); 7.499 (1.1); 7.498 (1.2); 7.491 (1.1); 7.490 (1.1); 7.479 (1.1); 7.478 (1.1); 7.402 (1.2); 7.384 (1.9);
7.365 (0.8); 6.528 (3.2); 5.758 (3.2); 5.382 (7.8); 3.332 (36.7); 2.526 (0.4); 2.512 (8.5); 2.508 (17.0); 2.503 (22.4); 2.499 (16.4);
2.495 (8.1); 2.410 (12.0); 2.257 (16.0); 0.008 (0.5); 0.000 (15.4); −0.009 (0.7)
Compound No. I-1-304: ¹H NMR (400.0 MHz, d₆-DMSO):
10.946 (3.2); 9.074 (2.8); 9.070 (2.8); 8.713 (2.1); 8.709 (2.2); 8.701 (2.3); 8.697 (2.2); 8.293 (1.1); 8.288 (1.6); 8.283 (1.1); 8.273
(1.2); 8.268 (1.7); 8.263 (1.1); 8.131 (7.4); 7.953 (0.5); 7.640 (1.5); 7.635 (2.2); 7.629 (1.1); 7.618 (4.7); 7.614 (5.4); 7.609
(2.7); 7.606 (2.7); 7.599 (1.1); 7.589 (5.0); 7.584 (1.8); 7.574 (1.6); 7.568 (3.0); 7.563 (2.7); 7.558 (1.4); 7.555 (1.0); 7.546 (1.8);
7.537 (0.4); 7.533 (0.5); 7.529 (0.6); 7.525 (0.4); 7.512 (1.7); 7.499 (1.7); 7.492 (1.6); 7.480 (1.5); 7.479 (1.5); 6.445 (4.4); 5.436
(10.0); 3.328 (38.2); 2.891 (3.5); 2.732 (2.9); 2.511 (14.3); 2.507 (27.9); 2.502 (36.1); 2.498 (26.4); 2.493 (13.1); 2.246 (16.0);
0.008 (0.7); 0.000 (17.6); −0.008 (0.8)
Compound No. I-1-305: mass (m/z): 519.2 (M + H)⁺; ¹H NMR (DMSO-d6) 2.27 (s, 3H), 5.35 (s, 2H), 5.68 (s, 2H),
7.26 (m, 1H), 7.53-7.63 (m, 6H), 7.76 (m, 1H), 7.95-8.0 (m, 4H), 8.45 (m, 1H), 8.55 (m, 1H).
Compound No. I-1-306: ¹H NMR (600.1 MHz, d₆-DMSO):
11.340 (3.6); 9.154 (2.8); 9.151 (2.8); 8.890 (2.9); 8.886 (2.9); 8.756 (2.0); 8.754 (2.1); 8.748 (2.1); 8.746 (2.0); 8.367 (1.2); 8.364
(1.7); 8.361 (1.2); 8.354 (1.2); 8.350 (1.8); 8.347 (1.1); 8.174 (2.0); 8.170 (1.9); 8.160 (2.2); 8.155 (2.2); 7.796 (2.2); 7.782
(2.2); 7.558 (1.5); 7.550 (1.5); 7.545 (1.5); 7.537 (1.5); 7.213 (3.4); 7.171 (3.4); 6.851 (4.3); 3.351 (101.8); 2.524 (0.3); 2.521 (0.4);
2.512 (8.0); 2.509 (16.5); 2.506 (22.4); 2.503 (16.4); 2.500 (7.8); 2.465 (16.0); 0.000 (0.9)
Compound No. I-1-307: ¹H NMR(601.6 MHz, d₆-DMSO):
19.963 (0.3); 11.284 (5.2); 9.139 (4.6); 9.135 (4.8); 8.767 (3.3); 8.765 (3.6); 8.759 (3.8); 8.757 (3.6); 8.348 (1.7); 8.344 (2.6); 8.341
(1.9); 8.334 (1.9); 8.331 (2.8); 8.328 (2.0); 8.322 (0.8); 8.230 (2.7); 8.215 (2.8); 7.568 (2.4); 7.560 (2.4); 7.554 (2.5); 7.546
(2.3); 6.785 (6.1); 6.724 (0.5); 6.566 (1.8); 6.551 (1.9); 6.525 (2.2); 6.510 (2.1); 5.762 (1.0); 4.706 (0.7); 4.693 (1.1); 4.679 (1.1);
4.666 (0.7); 4.465 (0.4); 4.452 (1.2); 4.439 (2.2); 4.426 (2.6); 4.412 (2.2); 4.400 (1.3); 4.388 (0.4); 3.343 (60.8); 2.615 (0.6); 2.544
(22.3); 2.533 (2.1); 2.522 (1.2); 2.519 (1.2); 2.507 (63.1); 2.504 (87.4); 2.501 (64.7); 2.388 (0.5); 2.239 (0.4); 1.411 (10.6);
1.399 (10.6); 1.362 (0.8); 1.269 (0.8); 1.223 (12.9); 1.218 (14.5); 1.211 (13.4); 1.206 (16.0); 1.195 (2.2); 0.005 (2.1); 0.000 (64.3);
−0.006 (2.5)
Compound No. I-1-308: ¹H NMR (400.0 MHz, d₆-DMSO):
11.229 (2.4); 9.118 (2.1); 9.113 (2.2); 8.764 (1.4); 8.760 (1.5); 8.752 (1.5); 8.748 (1.5); 8.333 (0.8); 8.328 (1.3); 8.323 (0.9); 8.313
(0.9); 8.308 (1.4); 8.303 (0.9); 8.094 (0.6); 8.080 (1.3); 8.065 (0.6); 7.566 (1.2); 7.554 (1.1); 7.546 (1.1); 7.534 (1.1); 6.722
(3.2); 5.757 (4.3); 4.038 (0.4); 4.020 (0.4); 3.708 (0.5); 3.690 (1.1); 3.674 (2.0); 3.659 (1.6); 3.644 (0.5); 3.639 (0.3); 3.331 (25.8);
3.126 (0.4); 3.110 (0.6); 3.092 (0.9); 3.077 (0.9); 3.059 (0.6); 2.944 (0.6); 2.929 (1.3); 2.912 (0.8); 2.896 (1.0); 2.881 (0.4); 2.612
(16.0); 2.540 (10.9); 2.526 (0.5); 2.507 (17.8); 2.503 (23.7); 2.498 (17.8); 2.236 (0.5); 1.989 (1.8); 1.193 (0.5); 1.175 (1.0);
1.157 (0.5); 0.008 (0.4); 0.000 (11.5); −0.008 (0.5)
Compound No. I-1-309: ¹H NMR (400.0 MHz, d₆-DMSO):
11.224 (3.5); 9.114 (3.0); 9.109 (3.0); 8.765 (2.0); 8.761 (2.0); 8.753 (2.1); 8.749 (1.9); 8.329 (1.2); 8.324 (1.7); 8.319 (1.3); 8.309
(1.3); 8.304 (1.9); 8.299 (1.3); 8.044 (0.8); 8.029 (1.7); 8.014 (0.9); 7.568 (1.5); 7.556 (1.5); 7.548 (1.5); 7.536 (1.5); 7.535
(1.4); 6.727 (3.9); 6.486 (0.3); 5.755 (5.1); 4.391 (0.6); 3.738 (1.0); 3.721 (2.8); 3.706 (3.0); 3.690 (1.3); 3.642 (0.3); 3.630 (0.5);
3.613 (0.4); 3.568 (6.0); 3.435 (2.1); 3.419 (4.0); 3.402 (1.8); 3.355 (0.4); 3.327 (58.1); 3.077 (0.8); 3.059 (16.0); 3.052 (2.2);
2.913 (2.1); 2.544 (12.9); 2.507 (31.5); 2.502 (40.8); 2.498 (30.4); 2.457 (0.7); 2.323 (1.4); 2.203 (0.5); 2.164 (1.2); 2.074 (2.0);
1.235 (0.6); 0.000 (48.5); −0.008 (2.5)
Compound No. I-1-310: ¹H NMR (400.0 MHz, d₆-DMSO):
9.127 (7.4); 9.123 (7.4); 8.764 (5.1); 8.760 (5.6); 8.752 (5.5); 8.748 (5.5); 8.342 (2.9); 8.337 (4.2); 8.332 (3.1); 8.322 (3.3); 8.316
(7.1); 8.313 (3.4); 7.725 (1.7); 7.711 (3.2); 7.696 (1.8); 7.565 (3.9); 7.553 (3.9); 7.547 (3.8); 7.545 (3.9); 7.535 (3.6); 7.533 (3.8);
6.737 (10.7); 6.735 (10.9); 6.030 (0.8); 6.015 (1.5); 6.000 (0.8); 5.756 (4.2); 3.771 (3.3); 3.759 (3.9); 3.750 (11.6); 3.744 (12.3);
3.730 (5.0); 3.722 (13.1); 3.716 (14.8); 3.708 (7.9); 3.701 (3.5); 3.686 (11.0); 3.679 (11.7); 3.658 (6.2); 3.630 (12.3); 3.627
(12.9); 3.625 (12.6); 3.602 (11.0); 3.596 (11.7); 3.582 (4.2); 3.575 (4.8); 3.568 (9.1); 3.562 (5.0); 3.553 (3.4); 3.547 (4.8); 3.541
(13.8); 3.534 (11.4); 3.518 (1.8); 3.512 (8.2); 3.506 (7.8); 3.493 (3.4); 3.486 (3.4); 3.465 (4.8); 3.459 (4.8); 3.444 (11.4); 3.437
(11.0); 3.416 (14.0); 3.410 (12.2); 3.396 (4.4); 3.389 (7.7); 3.382 (10.7); 3.376 (7.7); 3.372 (6.9); 3.366 (8.7); 3.361 (7.5); 3.357
(10.1); 3.351 (10.8); 3.342 (9.6); 3.336 (9.3); 3.317 (6.5); 3.293 (5.0); 3.281 (8.3); 3.257 (7.4); 3.253 (6.6); 3.245 (2.7); 3.229
(5.1); 3.198 (13.7); 3.190 (2.7); 3.173 (12.0); 3.170 (14.6); 3.145 (9.4); 3.136 (1.9); 3.092 (0.7); 3.078 (0.9); 3.063 (0.8); 3.058
(1.1); 3.043 (1.5); 3.030 (1.0); 2.984 (0.9); 2.970 (1.5); 2.954 (1.0); 2.936 (0.9); 2.922 (0.6); 2.676 (0.6); 2.672 (0.9); 2.667 (0.7);
2.534 (37.9); 2.523 (7.6); 2.507 (92.0); 2.503 (111.4); 2.498 (82.1); 2.494 (44.4); 2.481 (17.5); 2.466 (16.0); 2.448 (5.1); 2.434
(4.7); 2.370 (0.4); 2.334 (0.7); 2.329 (0.8); 2.325 (0.7); 2.234 (1.6); 1.801 (0.5); 1.234 (0.4); 0.146 (0.4); 0.008 (3.0); 0.000
(87.8); −0.008 (4.4); −0.150 (0.4)
Compound No. I-1-311: ¹H NMR (400.0 MHz, d₆-DMSO):
11.289 (13.0); 9.133 (11.0); 9.128 (11.0); 8.765 (7.2); 8.761 (7.0); 8.753 (7.6); 8.750 (6.7); 8.346 (4.4); 8.342 (6.3); 8.326 (4.8);
8.322 (6.7); 7.660 (3.4); 7.645 (5.9); 7.631 (3.4); 7.567 (5.9); 7.555 (6.0); 7.547 (5.9); 7.535 (5.5); 6.744 (16.0); 5.983 (0.9); 5.971
(1.5); 5.958 (0.9); 5.758 (6.2); 4.000 (1.2); 3.983 (3.6); 3.972 (4.2); 3.966 (4.3); 3.955 (3.9); 3.937 (1.4); 3.807 (2.5); 3.789
(5.6); 3.770 (7.0); 3.753 (4.3); 3.731 (2.3); 3.713 (1.0); 3.677 (3.1); 3.659 (6.5); 3.642 (5.7); 3.622 (3.3); 3.604 (2.2); 3.587 (1.7);
3.569 (0.7); 3.439 (2.1); 3.428 (2.5); 3.423 (2.6); 3.411 (2.7); 3.405 (3.4); 3.394 (3.8); 3.390 (3.6); 3.378 (2.9); 3.331 (51.6); 3.268
(3.0); 3.254 (3.9); 3.250 (3.5); 3.236 (4.7); 3.221 (2.6); 3.216 (2.5); 3.203 (2.0); 3.147 (0.5); 3.133 (0.7); 3.128 (0.6); 3.123
(0.7); 3.113 (1.0); 3.104 (1.1); 3.098 (1.0); 3.094 (1.0); 3.090 (1.0); 3.079 (0.6); 3.024 (0.6); 3.015 (0.8); 3.009 (1.1); 3.001 (1.1);
2.994 (0.8); 2.985 (0.7); 2.975 (0.7); 2.967 (0.6); 2.952 (0.3); 2.673 (0.6); 2.540 (52.6); 2.507 (70.2); 2.504 (85.1); 2.330 (0.6);
1.977 (1.0); 1.958 (1.9); 1.945 (2.6); 1.940 (2.7); 1.934 (2.3); 1.925 (3.4); 1.912 (3.0); 1.895 (2.7); 1.879 (2.2); 1.863 (4.5); 1.850
(6.8); 1.832 (6.7); 1.829 (6.7); 1.813 (5.8); 1.803 (3.7); 1.798 (4.1); 1.783 (2.7); 1.773 (1.7); 1.744 (0.6); 1.732 (0.3); 1.602
(1.6); 1.585 (3.1); 1.571 (2.5); 1.564 (3.0); 1.555 (3.2); 1.536 (2.6); 1.518 (1.0); 1.500 (0.6); 1.484 (0.8); 1.471 (0.8); 1.461 (0.8);
1.454 (1.3); 1.445 (0.6); 1.436 (0.9); 0.000 (58.8)
Compound No. I-1-312: ¹H NMR(601.6 MHz, d₆-DMSO):
19.978 (0.4); 11.198 (12.3); 9.118 (11.3); 9.115 (11.4); 8.760 (8.0); 8.757 (8.5); 8.752 (8.6); 8.749 (8.5); 8.324 (6.4); 8.313 (4.3);
8.310 (6.6); 8.307 (4.4); 7.866 (6.1); 7.860 (5.4); 7.560 (6.2); 7.552 (6.2); 7.547 (6.2); 7.539 (6.0); 6.711 (16.0); 6.029 (1.3);
5.762 (1.9); 3.352 (29.0); 3.350 (38.1); 2.765 (0.7); 2.759 (2.3); 2.753 (3.6); 2.747 (5.2); 2.741 (5.3); 2.735 (3.8); 2.729 (2.3); 2.723
(0.9); 2.617 (0.5); 2.532 (59.2); 2.508 (63.5); 2.505 (86.4); 2.502 (64.2); 2.422 (0.4); 2.403 (0.5); 2.392 (1.4); 2.387 (1.4); 2.381
(0.9); 2.239 (0.4); 1.232 (0.4); 0.732 (2.9); 0.724 (9.0); 0.720 (12.0); 0.712 (12.0); 0.709 (9.6); 0.701 (3.9); 0.686 (0.6); 0.675
(0.5); 0.645 (0.5); 0.638 (0.5); 0.618 (4.0); 0.611 (11.5); 0.606 (11.8); 0.601 (9.8); 0.592 (2.9); 0.551 (0.8); 0.543 (2.4); 0.539
(3.1); 0.532 (3.1); 0.528 (2.4); 0.520 (0.9); 0.330 (0.9); 0.323 (2.7); 0.319 (2.7); 0.313 (2.5); 0.305 (0.7); 0.000 (59.4); −0.006
(2.5)
Compound No. I-1-313: ¹H NMR (400.0 MHz, d₆-DMSO):
11.283 (5.9); 9.130 (5.1); 9.125 (5.1); 9.124 (5.1); 8.764 (3.6); 8.760 (4.0); 8.752 (3.9); 8.748 (4.0); 8.346 (2.0); 8.341 (3.0); 8.336
(2.2); 8.326 (2.2); 8.321 (3.2); 8.316 (2.4); 7.602 (1.5); 7.587 (2.6); 7.573 (1.5); 7.565 (2.9); 7.564 (3.1); 7.552 (2.9); 7.545
(2.6); 7.544 (2.8); 7.533 (2.4); 7.532 (2.7); 6.744 (7.2); 6.743 (7.6); 5.757 (16.0); 3.901 (1.7); 3.874 (1.9); 3.869 (1.8); 3.456 (0.5);
3.447 (0.8); 3.441 (0.9); 3.438 (0.9); 3.428 (1.4); 3.423 (1.6); 3.414 (1.0); 3.402 (2.5); 3.390 (1.7); 3.380 (1.8); 3.373 (2.5); 3.363
(2.0); 3.356 (3.7); 3.347 (3.0); 3.333 (55.2); 3.238 (1.0); 3.225 (1.8); 3.220 (1.5); 3.206 (2.2); 3.192 (1.3); 3.186 (0.9); 3.173
(1.0); 2.536 (25.1); 2.522 (1.3); 2.513 (14.2); 2.508 (29.3); 2.504 (39.8); 2.499 (30.3); 2.495 (15.6); 1.794 (1.0); 1.769 (2.0); 1.600
(1.5); 1.567 (1.7); 1.519 (0.3); 1.488 (1.7); 1.464 (5.2); 1.437 (1.9); 1.415 (0.8); 1.405 (0.6); 1.252 (0.7); 1.222 (1.3); 1.193
(1.1); 1.173 (0.4); 1.164 (0.4); 1.111 (0.3); 0.000 (1.9)
Compound No. I-1-314: ¹H NMR (400.0 MHz, d₆-DMSO):
9.123 (4.0); 9.119 (4.0); 9.118 (3.9); 8.763 (2.9); 8.759 (3.1); 8.751 (3.1); 8.747 (3.1); 8.337 (1.6); 8.332 (2.3); 8.327 (1.8); 8.318
(9.6); 8.312 (2.7); 8.307 (1.8); 7.822 (0.9); 7.808 (1.8); 7.793 (1.0); 7.568 (2.1); 7.566 (2.2); 7.556 (2.1); 7.554 (2.1); 7.548 (2.1);
7.546 (2.1); 7.536 (2.0); 7.534 (2.0); 7.174 (2.1); 7.168 (0.7); 7.155 (2.1); 7.152 (3.1); 7.140 (0.7); 7.134 (2.3); 6.775 (1.0); 6.772
(0.9); 6.758 (4.2); 6.755 (4.7); 6.736 (5.4); 6.706 (5.8); 6.704 (5.9); 5.757 (1.1); 3.573 (1.5); 3.559 (1.5); 3.537 (1.5); 3.531
(1.5); 3.458 (1.2); 3.433 (1.2); 3.417 (1.0); 3.321 (0.7); 3.236 (2.1); 3.220 (4.0); 3.202 (4.1); 3.186 (2.1); 3.169 (0.4); 2.944 (0.8);
2.927 (1.4); 2.912 (1.4); 2.895 (1.1); 2.863 (0.7); 2.672 (0.4); 2.529 (21.1); 2.512 (25.3); 2.508 (49.1); 2.503 (65.6); 2.499 (50.8);
2.494 (30.1); 2.477 (4.1); 2.455 (0.6); 2.436 (0.4); 2.408 (1.1); 2.392 (1.1); 2.334 (0.4); 2.330 (0.5); 2.325 (0.4); 2.234 (0.5);
1.587 (0.5); 1.569 (2.2); 1.550 (4.2); 1.532 (4.3); 1.514 (2.4); 1.496 (0.6); 1.393 (0.9); 1.375 (3.2); 1.358 (5.8); 1.340 (5.7); 1.322
(3.1); 1.303 (0.7); 0.907 (7.5); 0.889 (15.6); 0.870 (7.2); 0.858 (7.5); 0.839 (16.0); 0.820 (12.2); 0.801 (3.2); 0.374 (0.3); 0.364
(0.9); 0.360 (1.0); 0.354 (0.5); 0.350 (0.5); 0.344 (1.0); 0.340 (0.9); 0.330 (0.4); 0.091 (0.8); 0.081 (0.8); 0.000 (5.3)
Compound No. I-1-315: ¹H NMR (400.0 MHz, d₆-DMSO):
9.124 (4.2); 9.120 (4.1); 9.118 (4.0); 8.764 (2.9); 8.760 (3.2); 8.752 (3.1); 8.748 (3.1); 8.337 (1.7); 8.332 (2.4); 8.327 (1.7); 8.317
(2.1); 8.312 (2.5); 8.307 (1.7); 7.800 (1.1); 7.785 (2.2); 7.770 (1.1); 7.567 (2.1); 7.566 (2.2); 7.555 (2.1); 7.554 (2.2); 7.547 (2.1);
7.546 (2.1); 7.535 (2.0); 7.534 (2.1); 6.704 (5.9); 6.703 (6.1); 5.716 (0.3); 5.702 (0.6); 5.688 (0.4); 3.335 (2.7); 3.276 (2.0); 3.258
(4.5); 3.243 (4.4); 3.225 (1.9); 2.979 (0.7); 2.963 (1.9); 2.948 (1.7); 2.931 (0.9); 2.528 (21.4); 2.513 (11.0); 2.508 (22.6); 2.504
(29.9); 2.499 (22.2); 2.495 (11.2); 1.547 (0.8); 1.529 (2.5); 1.511 (3.7); 1.493 (2.8); 1.474 (1.1); 1.367 (0.7); 1.349 (2.5); 1.329
(4.1); 1.310 (4.4); 1.293 (2.9); 1.277 (1.3); 1.260 (1.3); 1.249 (0.7); 1.242 (1.0); 1.223 (0.6); 0.921 (8.0); 0.903 (16.0); 0.885
(7.0); 0.879 (3.7); 0.860 (5.9); 0.842 (2.4); 0.000 (2.4)
Compound No. I-1-316: ¹H NMR (400.0 MHz, d₆-DMSO):
9.131 (1.6); 9.127 (1.6); 8.765 (1.1); 8.761 (1.2); 8.753 (1.2); 8.749 (1.1); 8.345 (0.6); 8.340 (0.9); 8.335 (0.7); 8.325 (0.8); 8.319
(6.8); 7.833 (0.5); 7.568 (0.8); 7.556 (0.8); 7.548 (0.8); 7.536 (0.8); 7.172 (0.8); 7.150 (1.1); 7.132 (0.9); 6.772 (0.4); 6.770 (0.4);
6.755 (1.7); 6.735 (2.1); 6.721 (2.3); 6.720 (2.3); 3.480 (0.6); 3.169 (2.6); 3.139 (4.5); 3.123 (5.7); 3.109 (5.8); 3.086 (5.3); 2.875
(1.1); 2.859 (1.3); 2.843 (1.0); 2.804 (0.7); 2.677 (0.4); 2.673 (0.5); 2.668 (0.4); 2.535 (8.1); 2.508 (28.5); 2.504 (37.7); 2.499
(28.4); 2.395 (12.9); 2.379 (13.4); 2.335 (0.5); 2.330 (0.5); 2.231 (0.4); 1.070 (0.4); 1.064 (0.3); 1.052 (0.6); 1.040 (0.4); 1.032
(0.4); 0.867 (0.6); 0.848 (1.6); 0.835 (2.9); 0.819 (3.9); 0.803 (2.9); 0.791 (1.4); 0.788 (1.4); 0.771 (0.5); 0.479 (0.4); 0.469
(1.4); 0.464 (1.6); 0.454 (0.8); 0.449 (1.5); 0.444 (1.5); 0.434 (0.6); 0.380 (0.7); 0.375 (0.8); 0.366 (5.3); 0.355 (15.6); 0.351 (16.0);
0.346 (7.2); 0.341 (7.6); 0.335 (15.7); 0.331 (14.5); 0.321 (6.0); 0.304 (0.7); 0.285 (0.6); 0.272 (0.7); 0.259 (1.9); 0.250 (1.7);
0.247 (1.9); 0.235 (0.5); 0.131 (0.4); 0.110 (1.1); 0.095 (4.6); 0.082 (14.2); 0.071 (13.4); 0.059 (3.6); 0.000 (3.3)
Compound No. I-1-317: ¹H NMR (400.0 MHz, d₆-DMSO):
11.223 (0.5); 9.134 (4.5); 9.130 (4.7); 8.763 (3.3); 8.759 (3.6); 8.751 (3.6); 8.747 (3.5); 8.347 (1.8); 8.342 (2.7); 8.337 (1.9); 8.327
(2.0); 8.322 (2.9); 8.317 (2.3); 7.565 (2.6); 7.553 (2.5); 7.546 (2.5); 7.534 (2.4); 7.532 (2.3); 7.464 (2.4); 7.443 (2.4); 7.153
(0.4); 6.758 (0.6); 6.755 (0.6); 6.736 (0.7); 6.726 (6.8); 6.725 (6.6); 3.780 (0.6); 3.763 (1.3); 3.742 (1.5); 3.725 (1.3); 3.708 (0.6);
3.331 (45.5); 2.672 (0.3); 2.534 (24.4); 2.521 (1.5); 2.512 (19.1); 2.508 (39.2); 2.503 (52.0); 2.499 (38.2); 2.495 (19.1); 2.330
(0.4); 1.579 (0.6); 1.570 (0.6); 1.563 (2.2); 1.552 (2.0); 1.544 (3.3); 1.535 (2.6); 1.526 (2.3); 1.517 (2.3); 1.507 (0.7); 1.499 (1.0);
1.324 (0.4); 1.183 (15.1); 1.166 (14.9); 0.981 (1.2); 0.964 (1.2); 0.905 (7.6); 0.886 (16.0); 0.868 (6.8); 0.829 (0.5); 0.811 (1.0);
0.792 (0.4); 0.000 (4.0)
Compound No. I-1-318: ¹H NMR (400.0 MHz, d₆-DMSO):
9.135 (4.5); 9.130 (4.7); 8.762 (3.2); 8.758 (3.3); 8.750 (3.4); 8.746 (3.3); 8.346 (1.8); 8.342 (2.7); 8.337 (1.8); 8.327 (2.0); 8.322
(2.9); 8.318 (2.5); 7.565 (2.6); 7.553 (2.5); 7.545 (2.5); 7.533 (2.4); 7.475 (2.5); 7.453 (2.5); 7.175 (0.7); 7.153 (1.0); 7.135 (0.7);
6.776 (0.4); 6.758 (1.7); 6.756 (1.6); 6.737 (1.7); 6.723 (6.8); 3.874 (0.5); 3.855 (1.0); 3.838 (1.5); 3.822 (1.1); 3.802 (0.5); 3.334
(6.4); 2.751 (0.6); 2.735 (0.6); 2.532 (24.1); 2.512 (16.5); 2.508 (32.7); 2.504 (42.4); 2.499 (30.8); 1.571 (0.4); 1.565 (0.6);
1.553 (1.0); 1.544 (0.7); 1.539 (0.9); 1.530 (1.4); 1.516 (1.2); 1.510 (1.1); 1.496 (1.1); 1.483 (1.2); 1.479 (1.0); 1.467 (1.4); 1.464
(1.5); 1.447 (1.5); 1.432 (0.6); 1.429 (0.7); 1.414 (0.6); 1.374 (0.5); 1.365 (0.5); 1.355 (1.4); 1.348 (1.4); 1.336 (2.1); 1.330
(2.3); 1.323 (2.1); 1.316 (2.1); 1.312 (2.1); 1.307 (2.1); 1.301 (1.6); 1.289 (1.6); 1.283 (1.3); 1.273 (1.0); 1.265 (1.2); 1.257 (0.6);
1.248 (0.8); 1.240 (0.4); 1.223 (0.9); 1.209 (1.3); 1.184 (14.0); 1.167 (13.6); 0.980 (0.8); 0.964 (0.8); 0.950 (4.8); 0.934 (5.0);
0.909 (8.2); 0.890 (16.0); 0.872 (8.6); 0.855 (4.2); 0.837 (2.4); 0.000 (3.2)
Compound No. I-1-319: ¹H NMR (400.0 MHz, d₆-DMSO):
9.136 (0.8); 9.134 (0.9); 9.130 (0.9); 9.129 (0.8); 8.761 (0.7); 8.757 (0.7); 8.749 (0.7); 8.745 (0.7); 8.348 (0.4); 8.343 (0.5); 8.342
(0.5); 8.338 (0.4); 8.328 (0.4); 8.322 (0.6); 8.318 (0.7); 7.562 (0.5); 7.560 (0.5); 7.550 (0.5); 7.548 (0.5); 7.542 (0.5); 7.540 (0.5);
7.530 (0.5); 7.528 (0.5); 7.269 (0.9); 7.153 (0.4); 6.758 (0.6); 6.755 (0.6); 6.740 (1.4); 6.738 (1.5); 3.332 (5.4); 2.532 (4.3); 2.530
(4.4); 2.521 (0.3); 2.512 (3.4); 2.508 (7.3); 2.503 (9.9); 2.499 (7.2); 2.494 (3.5); 1.390 (16.0); 1.188 (2.3); 1.054 (1.4); 0.000
(0.8)
Compound No. I-1-320: ¹H NMR (400.0 MHz, d₆-DMSO):
11.201 (0.5); 9.127 (2.1); 9.126 (2.2); 9.122 (2.3); 8.764 (1.7); 8.760 (1.8); 8.752 (1.8); 8.748 (1.8); 8.339 (0.9); 8.335 (1.3); 8.329
(1.0); 8.319 (1.1); 8.315 (1.4); 8.314 (1.4); 8.309 (1.0); 7.795 (0.6); 7.780 (1.2); 7.765 (0.6); 7.567 (1.2); 7.565 (1.2); 7.555
(1.2); 7.553 (1.2); 7.547 (1.2); 7.545 (1.2); 7.535 (1.2); 7.533 (1.1); 6.717 (3.3); 6.715 (3.3); 3.334 (23.5); 3.105 (2.2); 3.089 (3.6);
3.073 (2.3); 2.817 (0.4); 2.802 (0.5); 2.786 (0.4); 2.531 (11.8); 2.530 (11.4); 2.513 (6.9); 2.508 (14.1); 2.504 (18.9); 2.499 (14.0);
2.495 (7.2); 1.874 (0.4); 1.857 (0.9); 1.840 (1.1); 1.823 (0.9); 1.807 (0.5); 0.903 (16.0); 0.886 (15.4); 0.824 (2.5); 0.808 (2.4);
0.000 (1.4)
Compound No. I-1-321: ¹H NMR (400.0 MHz, d₆-DMSO):
9.130 (0.5); 9.126 (0.5); 9.125 (0.5); 8.764 (0.4); 8.760 (0.4); 8.752 (0.4); 8.748 (0.4); 8.319 (0.6); 6.745 (0.7); 6.743 (0.8); 3.109
(0.9); 3.092 (0.9); 2.536 (2.6); 2.535 (2.6); 2.513 (1.1); 2.509 (2.4); 2.504 (3.2); 2.500 (2.4); 2.495 (1.2); 2.258 (3.9); 0.914 (8.9);
0.814 (1.8); 0.807 (16.0)
Compound No. I-1-322: ¹H NMR (400.0 MHz, d₆-DMSO):
11.188 (1.5); 9.117 (3.3); 9.113 (3.4); 8.760 (2.3); 8.756 (2.5); 8.748 (2.5); 8.744 (2.5); 8.329 (1.4); 8.324 (2.2); 8.319 (2.2); 8.309
(2.0); 8.303 (3.6); 8.300 (2.9); 8.286 (1.0); 7.561 (1.8); 7.549 (1.8); 7.541 (1.8); 7.530 (1.6); 7.529 (1.7); 7.358 (13.3); 7.347
(16.0); 7.326 (0.5); 7.312 (0.7); 7.303 (0.4); 7.299 (0.4); 7.293 (1.7); 7.283 (1.7); 7.271 (1.8); 7.259 (1.7); 7.250 (0.5); 7.241 (0.6);
6.724 (4.7); 6.722 (4.9); 4.457 (5.4); 4.441 (5.4); 4.242 (0.8); 4.227 (0.8); 3.343 (26.2); 2.542 (16.2); 2.541 (16.2); 2.512 (6.5);
2.508 (13.5); 2.504 (18.1); 2.499 (13.6); 2.495 (7.1); 2.239 (0.3)
Compound No. I-1-323: ¹H NMR (400.0 MHz, d₆-DMSO):
11.232 (12.8); 9.123 (10.7); 9.119 (10.9); 8.764 (7.6); 8.760 (8.5); 8.752 (8.2); 8.748 (8.5); 8.337 (4.3); 8.333 (6.3); 8.327 (4.7);
8.317 (5.2); 8.312 (6.9); 8.307 (5.1); 8.295 (3.3); 8.279 (6.9); 8.264 (3.4); 7.566 (5.6); 7.565 (5.8); 7.554 (5.6); 7.553 (5.8); 7.546
(5.6); 7.545 (5.7); 7.534 (5.3); 7.533 (5.5); 7.493 (6.1); 7.489 (5.1); 7.475 (8.3); 7.470 (8.4); 7.455 (0.4); 7.431 (5.4); 7.426
(5.1); 7.412 (8.3); 7.408 (8.5); 7.381 (2.6); 7.376 (3.6); 7.362 (9.1); 7.358 (8.5); 7.349 (9.9); 7.345 (12.4); 7.343 (13.4); 7.330 (7.6);
7.326 (7.6); 7.312 (2.4); 7.307 (2.4); 7.297 (1.6); 7.291 (1.3); 7.278 (1.4); 7.273 (1.3); 7.261 (0.7); 7.255 (0.6); 6.750 (15.5);
6.748 (16.0); 6.635 (0.8); 6.620 (1.6); 6.605 (0.8); 5.757 (2.0); 4.547 (15.5); 4.532 (15.5); 4.307 (4.6); 4.292 (4.6); 3.353 (0.6);
3.332 (126.1); 2.700 (0.3); 2.677 (0.5); 2.672 (0.8); 2.668 (0.6); 2.540 (53.1); 2.539 (53.3); 2.526 (2.9); 2.512 (38.3); 2.507 (79.4);
2.503 (106.6); 2.498 (79.6); 2.494 (40.4); 2.334 (0.5); 2.330 (0.7); 2.325 (0.5); 1.233 (0.6); 0.008 (1.1); 0.000 (31.3); −0.008
(1.3)
Compound No. I-1-324: ¹H NMR (400.0 MHz, d₆-DMSO):
11.173 (10.2); 9.114 (12.4); 8.760 (8.2); 8.749 (8.6); 8.471 (3.4); 8.456 (7.0); 8.440 (3.8); 8.327 (6.4); 8.307 (7.0); 7.566 (5.5);
7.554 (5.9); 7.547 (6.1); 7.534 (5.5); 7.406 (13.8); 7.386 (8.6); 7.367 (10.0); 7.344 (9.3); 7.341 (9.0); 7.324 (13.4); 7.306 (5.8);
6.720 (16.0); 5.757 (8.2); 4.453 (15.2); 4.438 (15.8); 3.333 (52.1); 2.672 (0.5); 2.533 (51.5); 2.503 (79.2); 2.500 (73.8); 2.330
(0.5); 0.000 (6.2)
Compound No. I-1-325: ¹H NMR (400.0 MHz, d₆-DMSO):
11.176 (2.2); 9.115 (5.5); 9.111 (5.6); 9.110 (5.5); 8.763 (4.0); 8.759 (4.6); 8.751 (4.3); 8.747 (4.5); 8.427 (1.6); 8.412 (3.4); 8.396
(1.7); 8.329 (2.2); 8.323 (3.2); 8.319 (2.5); 8.309 (2.5); 8.303 (3.5); 8.299 (2.6); 7.567 (2.8); 7.565 (3.1); 7.555 (2.8); 7.553
(3.0); 7.547 (2.8); 7.545 (3.0); 7.535 (2.7); 7.533 (2.9); 7.426 (5.6); 7.420 (2.6); 7.410 (3.4); 7.404 (16.0); 7.378 (14.2); 7.357 (6.2);
7.271 (1.8); 7.250 (1.3); 6.717 (7.9); 6.715 (8.4); 6.532 (0.5); 5.758 (0.7); 4.437 (7.9); 4.422 (7.8); 4.213 (1.5); 4.198 (1.5); 3.334
(68.0); 2.672 (0.4); 2.532 (26.7); 2.530 (27.5); 2.512 (19.7); 2.508 (42.4); 2.503 (58.0); 2.499 (43.6); 2.494 (22.2); 2.330
(0.4)
Compound No. I-1-326: ¹H NMR (400.0 MHz, d₆-DMSO):
11.213 (9.2); 9.121 (10.9); 9.117 (10.9); 9.115 (10.9); 8.763 (7.8); 8.759 (8.7); 8.751 (8.5); 8.747 (8.7); 8.334 (4.3); 8.329 (6.4);
8.324 (4.8); 8.314 (4.9); 8.309 (7.0); 8.304 (5.0); 8.278 (3.1); 8.263 (6.5); 8.247 (3.2); 7.564 (5.6); 7.563 (6.0); 7.551 (6.0); 7.545
(5.6); 7.543 (5.9); 7.533 (5.3); 7.531 (5.7); 7.436 (2.7); 7.433 (3.2); 7.418 (5.5); 7.413 (6.5); 7.398 (3.3); 7.394 (3.7); 7.374
(1.4); 7.370 (1.5); 7.360 (1.7); 7.356 (3.7); 7.341 (3.6); 7.335 (4.7); 7.331 (3.1); 7.322 (2.7); 7.317 (2.8); 7.304 (0.4); 7.298 (0.9);
7.293 (0.8); 7.285 (0.6); 7.279 (0.7); 7.274 (0.4); 7.265 (0.4); 7.261 (0.3); 7.230 (5.6); 7.216 (6.2); 7.212 (4.4); 7.207 (6.0); 7.203
(6.7); 7.200 (10.3); 7.186 (3.7); 7.182 (6.9); 7.179 (4.4); 7.158 (1.2); 7.149 (0.8); 7.142 (0.6); 7.140 (0.5); 7.129 (0.5); 6.735
(15.4); 6.734 (16.0); 6.510 (0.3); 6.495 (0.7); 6.480 (0.3); 4.521 (13.9); 4.506 (13.9); 4.276 (1.7); 4.261 (1.7); 3.339 (70.8); 2.678
(0.3); 2.673 (0.4); 2.669 (0.3); 2.536 (53.9); 2.522 (2.4); 2.513 (23.0); 2.509 (48.5); 2.504 (65.8); 2.499 (48.9); 2.495 (24.7);
2.331 (0.4); 2.240 (0.6)
Compound No. I-1-327: ¹H NMR (400.0 MHz, d₆-DMSO):
11.186 (2.6); 9.126 (10.7); 9.121 (11.0); 8.771 (6.4); 8.767 (7.6); 8.759 (7.0); 8.755 (7.5); 8.536 (0.6); 8.456 (3.1); 8.440 (6.4);
8.425 (3.3); 8.333 (6.1); 8.329 (4.5); 8.318 (4.1); 8.313 (6.5); 8.309 (4.6); 7.630 (0.4); 7.573 (5.7); 7.561 (5.8); 7.553 (5.8); 7.541
(5.6); 7.521 (0.4); 7.431 (2.3); 7.410 (5.6); 7.395 (6.3); 7.375 (3.8); 7.347 (0.8); 7.342 (0.8); 7.327 (0.6); 7.207 (8.2); 7.187 (12.4);
7.162 (5.3); 7.129 (3.0); 7.123 (2.6); 7.106 (6.3); 7.086 (3.4); 7.066 (1.2); 7.043 (1.1); 6.729 (16.0); 6.611 (0.4); 6.596 (0.7);
6.581 (0.4); 4.813 (1.0); 4.474 (14.9); 4.459 (14.9); 4.260 (2.1); 4.245 (2.0); 3.347 (71.4); 2.679 (0.4); 2.542 (53.5); 2.511 (67.4);
2.507 (54.1); 2.378 (0.3); 2.337 (0.4); 2.245 (0.5)
Compound No. I-1-328: ¹H NMR (400.0 MHz, d₆-DMSO):
11.174 (5.4); 9.114 (9.1); 9.109 (9.1); 8.762 (6.1); 8.758 (6.6); 8.750 (6.5); 8.746 (6.5); 8.376 (2.6); 8.361 (5.5); 8.346 (2.7); 8.327
(3.6); 8.322 (5.5); 8.317 (3.8); 8.307 (3.8); 8.302 (5.7); 8.298 (3.7); 7.564 (4.9); 7.552 (4.9); 7.544 (4.8); 7.532 (4.6); 7.411
(6.7); 7.396 (8.1); 7.389 (9.3); 7.375 (8.2); 7.296 (0.8); 7.281 (1.0); 7.274 (1.2); 7.260 (1.1); 7.205 (1.2); 7.197 (9.4); 7.192 (3.2);
7.181 (3.7); 7.175 (16.0); 7.170 (3.8); 7.158 (2.9); 7.153 (8.9); 7.136 (0.6); 7.131 (2.1); 7.109 (1.0); 6.714 (13.5); 6.483 (0.4);
6.468 (0.6); 6.452 (0.3); 5.758 (1.0); 4.432 (12.6); 4.416 (12.5); 4.208 (1.8); 4.193 (1.8); 3.335 (77.3); 2.672 (0.6); 2.668 (0.4);
2.535 (47.3); 2.508 (62.3); 2.503 (81.7); 2.499 (60.2); 2.335 (0.4); 2.330 (0.5); 2.326 (0.4)
Compound No. I-1-329: ¹H NMR (400.0 MHz, d₆-DMSO):
11.282 (2.0); 9.134 (10.1); 9.130 (10.1); 9.129 (10.0); 9.112 (0.4); 8.768 (7.2); 8.764 (8.0); 8.756 (7.8); 8.752 (7.9); 8.549 (5.8);
8.538 (5.9); 8.537 (6.0); 8.510 (0.9); 8.503 (1.4); 8.498 (1.2); 8.493 (1.4); 8.491 (1.4); 8.465 (0.3); 8.450 (0.6); 8.441 (0.6); 8.436
(0.6); 8.429 (0.5); 8.423 (0.5); 8.406 (3.3); 8.392 (7.0); 8.378 (3.5); 8.351 (4.1); 8.345 (6.1); 8.341 (4.3); 8.331 (4.5); 8.325
(6.6); 8.321 (4.7); 8.302 (0.4); 8.261 (0.6); 8.251 (0.6); 7.822 (3.7); 7.817 (3.8); 7.802 (7.4); 7.798 (7.6); 7.783 (4.6); 7.779 (4.9);
7.758 (1.1); 7.753 (1.1); 7.746 (0.7); 7.741 (0.7); 7.739 (0.8); 7.734 (0.9); 7.729 (1.5); 7.725 (1.6); 7.717 (0.9); 7.712 (0.9); 7.707
(0.6); 7.702 (0.4); 7.698 (0.6); 7.693 (0.8); 7.687 (0.7); 7.682 (0.6); 7.676 (0.5); 7.669 (0.8); 7.656 (1.4); 7.641 (0.8); 7.636
(1.2); 7.631 (0.9); 7.623 (0.5); 7.617 (0.6); 7.611 (0.7); 7.606 (0.5); 7.592 (0.3); 7.587 (0.3); 7.579 (0.4); 7.569 (5.7); 7.557 (5.7);
7.549 (5.6); 7.539 (5.1); 7.537 (5.5); 7.403 (8.2); 7.383 (7.6); 7.358 (1.1); 7.353 (0.9); 7.346 (0.6); 7.339 (0.7); 7.334 (0.7); 7.324
(4.7); 7.311 (5.3); 7.307 (5.4); 7.293 (4.5); 7.281 (0.9); 7.275 (0.9); 7.269 (1.0); 7.264 (1.4); 7.261 (1.2); 7.252 (1.3); 7.249
(1.3); 7.245 (1.1); 7.230 (1.0); 7.221 (0.6); 7.209 (0.8); 7.194 (0.9); 7.190 (0.9); 7.179 (1.7); 7.173 (1.0); 7.161 (1.6); 7.149 (0.8);
7.131 (0.4); 7.112 (0.5); 7.104 (0.3); 7.092 (0.5); 7.051 (0.4); 7.031 (0.4); 7.001 (0.6); 6.989 (0.6); 6.983 (0.7); 6.970 (0.5); 6.946
(0.8); 6.926 (0.7); 6.758 (14.7); 6.756 (15.3); 5.060 (0.3); 4.656 (0.4); 4.636 (0.4); 4.620 (0.4); 4.599 (15.8); 4.585 (16.0); 4.565
(0.5); 4.368 (1.1); 4.352 (1.0); 4.340 (2.0); 4.325 (2.0); 4.272 (0.3); 4.146 (0.4); 4.014 (0.4); 3.998 (0.4); 3.940 (0.4); 3.929
(0.6); 3.910 (0.5); 3.873 (1.6); 3.869 (1.7); 3.614 (0.5); 3.605 (0.5); 3.546 (0.5); 3.521 (0.5); 3.484 (0.4); 3.336 (185.5); 2.677
(0.5); 2.673 (0.7); 2.668 (0.5); 2.550 (51.4); 2.526 (1.5); 2.521 (2.2); 2.512 (39.1); 2.508 (83.3); 2.503 (113.3); 2.499 (84.5); 2.495
(42.8); 2.385 (0.4); 2.335 (0.6); 2.330 (0.8); 2.326 (0.6); 2.237 (1.1); 1.233 (0.4); 0.000 (0.4)
Compound No. I-1-330: ¹H NMR (400.0 MHz, d₆-DMSO):
12.153 (0.6); 11.164 (2.5); 10.930 (0.7); 9.113 (3.3); 9.107 (3.5); 8.763 (1.8); 8.759 (1.9); 8.751 (1.9); 8.747 (1.9); 8.726 (0.8);
8.723 (0.9); 8.715 (0.8); 8.711 (0.9); 8.579 (2.2); 8.574 (2.3); 8.490 (2.2); 8.487 (2.2); 8.478 (2.2); 8.475 (3.1); 8.459 (0.8); 8.326
(1.4); 8.321 (2.1); 8.316 (1.7); 8.306 (1.6); 8.301 (2.3); 8.296 (1.5); 7.777 (0.8); 7.772 (1.3); 7.767 (0.9); 7.757 (0.9); 7.752 (1.5);
7.748 (1.0); 7.565 (1.4); 7.553 (1.3); 7.545 (1.3); 7.533 (1.8); 7.519 (0.6); 7.511 (0.7); 7.499 (0.6); 7.394 (1.3); 7.382 (1.3); 7.374
(1.3); 7.362 (1.2); 6.713 (3.6); 6.712 (3.7); 6.419 (0.5); 4.477 (3.6); 4.462 (3.6); 4.119 (1.6); 4.106 (4.8); 4.092 (4.9); 4.079
(1.7); 3.334 (57.2); 3.177 (16.0); 3.164 (15.6); 2.532 (12.8); 2.521 (1.2); 2.512 (13.0); 2.508 (27.1); 2.503 (36.6); 2.499 (27.3);
2.495 (13.9); 2.325 (0.3); 2.236 (5.8); 1.234 (0.8); 0.000 (9.2); −0.008 (0.4)
Compound No. I-1-331: ¹H NMR (400.0 MHz, d₆-DMSO):
11.265 (5.4); 9.136 (6.2); 9.131 (6.4); 8.763 (4.2); 8.759 (4.6); 8.751 (4.5); 8.747 (4.5); 8.346 (2.3); 8.342 (3.6); 8.337 (2.5); 8.326
(2.6); 8.322 (3.9); 8.317 (2.8); 7.997 (3.8); 7.977 (3.9); 7.563 (3.4); 7.551 (3.4); 7.543 (3.4); 7.531 (3.2); 7.434 (6.0); 7.415
(10.6); 7.382 (6.1); 7.364 (9.9); 7.345 (5.0); 7.292 (3.5); 7.274 (4.9); 7.256 (1.7); 6.740 (9.1); 5.011 (0.6); 4.993 (2.3); 4.975 (3.3);
4.956 (2.3); 4.939 (0.6); 3.335 (43.5); 2.673 (0.5); 2.542 (0.5); 2.512 (47.4); 2.504 (45.8); 2.499 (34.0); 2.239 (0.4); 1.537 (16.0);
1.519 (15.9); 1.300 (0.4); 1.297 (0.4); 0.000 (3.6)
Compound No. I-1-332: ¹H NMR (400.0 MHz, d₆-DMSO):
11.252 (6.1); 9.134 (7.2); 9.130 (7.2); 8.763 (4.6); 8.761 (4.8); 8.752 (4.9); 8.749 (4.7); 8.341 (4.1); 8.320 (4.4); 8.069 (4.4); 8.050
(4.5); 7.566 (3.6); 7.553 (3.8); 7.546 (3.8); 7.534 (3.4); 7.488 (5.3); 7.474 (6.8); 7.467 (7.3); 7.453 (6.0); 7.205 (6.1); 7.183
(10.9); 7.161 (5.2); 6.733 (10.0); 5.015 (0.7); 4.998 (2.5); 4.980 (3.7); 4.962 (2.5); 4.944 (0.7); 3.333 (32.1); 2.673 (0.5); 2.668
(0.5); 2.504 (94.2); 2.330 (0.5); 1.524 (16.0); 1.507 (15.9); 0.000 (15.0)
Compound No. I-1-333: ¹H NMR (400.0 MHz, d₆-DMSO):
11.380 (6.0); 9.157 (5.2); 9.152 (5.3); 8.858 (15.5); 8.846 (16.0); 8.834 (0.5); 8.796 (0.9); 8.783 (1.0); 8.773 (4.2); 8.769 (4.3);
8.761 (4.4); 8.757 (4.2); 8.373 (2.1); 8.369 (3.1); 8.364 (2.2); 8.354 (2.3); 8.348 (3.3); 8.344 (2.3); 8.250 (3.5); 8.230 (3.6); 8.052
(0.3); 7.573 (2.9); 7.561 (2.8); 7.554 (2.7); 7.553 (2.8); 7.542 (2.6); 7.541 (2.7); 7.493 (3.9); 7.481 (7.6); 7.468 (3.8); 7.410 (0.5);
6.785 (7.5); 6.783 (7.7); 5.758 (4.4); 5.130 (0.6); 5.113 (2.2); 5.095 (3.1); 5.077 (2.4); 5.059 (0.7); 3.333 (65.9); 2.674 (0.4);
2.544 (26.4); 2.527 (1.2); 2.513 (19.5); 2.509 (40.5); 2.504 (54.3); 2.500 (40.3); 2.496 (20.4); 2.331 (0.4); 2.227 (0.8); 1.852 (1.2);
1.575 (0.7); 1.561 (15.2); 1.544 (15.0); 1.412 (1.2); 1.394 (1.2); 1.382 (0.6); 1.365 (0.6)
Compound No. I-1-334: ¹H NMR (400.0 MHz, d₆-DMSO):
11.269 (1.3); 9.141 (1.5); 9.137 (1.9); 9.130 (1.1); 8.771 (2.1); 8.768 (1.0); 8.758 (2.0); 8.488 (0.7); 8.473 (0.3); 8.352 (0.4); 8.347
(0.6); 8.342 (0.4); 8.332 (0.5); 8.327 (0.7); 8.322 (0.4); 7.576 (0.5); 7.564 (0.6); 7.557 (0.5); 7.545 (0.5); 7.543 (0.5); 7.508
(0.7); 7.506 (0.8); 7.495 (0.7); 7.493 (0.7); 6.755 (1.5); 4.596 (1.6); 4.581 (1.6); 3.334 (8.3); 3.098 (1.1); 3.085 (1.5); 3.079 (3.5);
3.067 (3.8); 3.061 (3.8); 3.049 (3.7); 3.043 (1.6); 3.031 (1.2); 2.539 (5.1); 2.526 (0.5); 2.512 (6.5); 2.508 (12.6); 2.503 (16.3);
2.499 (12.2); 2.495 (6.3); 1.213 (8.1); 1.195 (16.0); 1.177 (7.7)
Compound No. I-1-335: ¹H NMR (400.0 MHz, d₆-DMSO):
11.333 (2.0); 9.159 (1.6); 9.155 (1.7); 8.876 (4.9); 8.863 (5.1); 8.773 (1.2); 8.769 (1.3); 8.761 (1.3); 8.757 (1.3); 8.666 (2.3); 8.376
(0.6); 8.371 (1.0); 8.366 (0.7); 8.356 (0.7); 8.351 (1.0); 8.346 (0.7); 7.575 (0.9); 7.573 (0.9); 7.562 (0.9); 7.561 (0.9); 7.555
(0.9); 7.553 (0.9); 7.543 (0.9); 7.541 (0.8); 7.479 (1.3); 7.467 (2.5); 7.455 (1.3); 6.757 (2.4); 3.327 (5.8); 2.530 (0.5); 2.526 (0.5);
2.508 (18.3); 2.504 (16.3); 2.499 (11.8); 2.495 (6.2); 1.781 (16.0); 1.765 (1.0); 1.652 (0.4); 0.008 (0.7); 0.000 (20.7); −0.008
(1.2)
Compound No. I-1-336: ¹H NMR (400.0 MHz, d₆-DMSO):
11.274 (0.4); 9.137 (0.6); 9.130 (0.4); 8.772 (0.5); 8.759 (0.6); 6.753 (0.5); 4.596 (0.5); 4.582 (0.5); 3.334 (2.2); 3.086 (1.0); 3.068
(3.1); 3.057 (3.7); 3.050 (3.5); 3.039 (3.5); 3.021 (1.2); 2.538 (1.6); 2.508 (5.8); 2.504 (7.7); 2.500 (6.2); 1.219 (8.5); 1.201
(16.0); 1.182 (8.1); 0.000 (6.5)
Compound No. I-1-337: ¹H NMR (400.0 MHz, d₆-DMSO):
11.015 (3.2); 9.090 (2.8); 9.086 (2.9); 8.715 (2.2); 8.711 (2.4); 8.703 (2.3); 8.699 (2.3); 8.317 (0.4); 8.310 (1.1); 8.305 (1.7); 8.300
(1.2); 8.290 (1.3); 8.285 (1.8); 8.280 (1.2); 7.515 (1.6); 7.503 (1.6); 7.496 (1.6); 7.484 (1.5); 7.483 (1.5); 7.293 (2.6); 7.274
(2.6); 7.235 (2.6); 7.207 (2.6); 6.524 (4.5); 5.757 (0.7); 5.223 (7.5); 3.818 (1.3); 3.792 (4.1); 3.766 (4.3); 3.754 (0.9); 3.740 (1.5);
3.325 (26.0); 2.672 (0.4); 2.525 (0.9); 2.511 (20.8); 2.507 (43.3); 2.502 (58.6); 2.498 (44.3); 2.494 (23.0); 2.460 (0.3); 2.394
(15.6); 2.333 (0.5); 2.329 (0.6); 2.325 (0.5); 2.320 (0.5); 2.301 (16.0); 2.279 (0.7); 2.247 (0.6); 0.008 (1.3); 0.000 (42.0); −0.008
(2.1)
Compound No. I-1-338: ¹H NMR (400.0 MHz, d₆-DMSO):
11.302 (1.4); 9.133 (1.8); 8.767 (1.2); 8.756 (1.2); 8.349 (1.0); 8.329 (1.0); 8.255 (0.6); 8.242 (1.1); 8.229 (0.6); 7.574 (0.8); 7.562
(0.9); 7.555 (0.9); 7.542 (0.8); 7.179 (2.5); 6.762 (2.4); 4.595 (2.5); 4.581 (2.5); 3.336 (26.3); 2.545 (7.6); 2.503 (18.9); 2.417
(16.0); 0.000 (15.7)
Compound No. I-1-339: ¹H NMR (400.0 MHz, d₆-DMSO):
11.071 (3.1); 9.111 (2.8); 9.107 (2.9); 8.722 (1.9); 8.719 (2.2); 8.710 (2.1); 8.707 (2.2); 8.325 (1.6); 8.310 (1.2); 8.305 (1.7); 8.108
(2.9); 8.085 (3.1); 7.727 (3.6); 7.553 (2.1); 7.550 (2.2); 7.531 (2.1); 7.526 (2.8); 7.511 (1.7); 7.504 (1.6); 7.491 (1.5); 6.596
(4.3); 5.757 (0.8); 5.479 (8.0); 3.326 (16.6); 2.672 (0.4); 2.508 (42.4); 2.503 (55.9); 2.499 (44.1); 2.320 (16.0); 1.233 (0.4); 0.007
(2.2); 0.000 (47.7)
Compound No. I-1-340: ¹H NMR (400.0 MHz, d₆-DMSO):
11.054 (3.3); 9.092 (2.9); 9.087 (2.9); 8.717 (2.1); 8.713 (2.2); 8.705 (2.2); 8.701 (2.2); 8.310 (1.1); 8.305 (1.7); 8.301 (1.2); 8.290
(1.2); 8.285 (1.8); 8.281 (1.2); 7.897 (3.6); 7.892 (3.7); 7.801 (3.6); 7.779 (4.1); 7.515 (1.6); 7.503 (1.6); 7.495 (1.6); 7.482
(3.8); 7.477 (2.6); 7.460 (2.2); 7.455 (2.2); 6.582 (4.6); 5.756 (0.8); 5.640 (10.5); 3.328 (35.5); 2.508 (35.0); 2.503 (46.1); 2.499
(34.5); 2.391 (16.0); 2.160 (0.6); 0.008 (2.3); 0.000 (53.8); −0.008 (2.5)
Compound No. I-1-341: ¹H NMR (400.0 MHz, d₆-DMSO):
11.070 (3.3); 9.106 (2.7); 9.101 (2.9); 8.717 (2.1); 8.713 (2.3); 8.705 (2.3); 8.701 (2.3); 8.370 (2.9); 8.349 (3.0); 8.324 (1.1); 8.319
(1.7); 8.314 (1.2); 8.304 (1.2); 8.299 (1.7); 8.294 (1.2); 8.000 (2.1); 7.979 (4.6); 7.957 (2.3); 7.798 (1.2); 7.794 (1.2); 7.781
(1.6); 7.777 (2.3); 7.773 (1.3); 7.760 (1.3); 7.756 (1.2); 7.625 (1.4); 7.622 (1.5); 7.605 (2.3); 7.588 (1.1); 7.585 (1.2); 7.519 (1.6);
7.506 (1.6); 7.499 (1.7); 7.487 (1.6); 7.122 (3.1); 7.101 (3.1); 6.602 (4.4); 5.757 (0.6); 5.520 (10.0); 3.328 (29.4); 2.525 (0.8);
2.512 (16.6); 2.508 (34.1); 2.503 (45.9); 2.499 (35.1); 2.494 (18.6); 2.346 (16.0); 2.330 (0.8); 1.990 (0.4); 0.008 (1.3); 0.000 (34.4);
−0.008 (1.9)
Compound No. I-1-342: ¹H NMR (400.0 MHz, d₆-DMSO):
11.052 (2.1); 10.612 (1.9); 9.091 (1.8); 9.086 (1.8); 9.023 (2.3); 9.019 (2.4); 8.772 (1.8); 8.768 (1.9); 8.760 (1.9); 8.756 (1.9); 8.717
(1.3); 8.713 (1.4); 8.705 (1.4); 8.701 (1.4); 8.309 (0.7); 8.305 (1.1); 8.300 (0.8); 8.290 (0.8); 8.284 (1.2); 8.280 (0.8); 8.234
(0.9); 8.229 (1.4); 8.224 (1.0); 8.214 (1.0); 8.209 (1.5); 8.204 (1.0); 7.981 (2.4); 7.977 (2.5); 7.934 (3.3); 7.930 (3.3); 7.788 (2.5);
7.767 (2.8); 7.728 (3.3); 7.707 (3.8); 7.572 (1.4); 7.560 (1.5); 7.554 (1.4); 7.552 (1.4); 7.542 (1.3); 7.540 (1.3); 7.515 (1.1); 7.503
(1.1); 7.495 (1.1); 7.485 (1.0); 7.483 (1.0); 7.462 (1.9); 7.457 (1.9); 7.441 (1.8); 7.436 (3.9); 7.431 (2.8); 7.415 (2.2); 7.410
(2.3); 6.580 (3.0); 6.226 (4.0); 5.757 (8.5); 5.632 (16.0); 3.326 (40.3); 2.676 (0.4); 2.672 (0.5); 2.667 (0.4); 2.525 (1.2); 2.511
(30.8); 2.507 (63.8); 2.503 (85.8); 2.498 (64.4); 2.494 (32.9); 2.390 (10.6); 2.334 (0.4); 2.329 (0.6); 2.325 (0.5); 2.295 (0.4); 2.158
(15.7); 2.071 (0.5); 1.235 (1.8)
Compound No. I-1-343: ¹H NMR (400.0 MHz, d₆-DMSO):
8.598 (4.2); 8.594 (4.2); 8.530 (2.7); 8.526 (3.0); 8.518 (3.0); 8.514 (3.0); 8.317 (14.9); 8.081 (3.3); 8.058 (4.0); 7.990 (3.1); 7.954
(1.7); 7.847 (3.1); 7.824 (4.6); 7.816 (2.0); 7.805 (1.8); 7.800 (2.8); 7.796 (1.9); 7.763 (2.0); 7.741 (1.8); 7.701 (0.4); 7.358
(2.2); 7.346 (2.3); 7.331 (5.3); 7.329 (5.2); 7.019 (2.0); 6.997 (1.9); 6.077 (1.2); 5.757 (0.4); 5.296 (10.0); 5.246 (10.8); 3.325 (62.5);
2.892 (12.6); 2.732 (10.2); 2.677 (0.7); 2.672 (1.0); 2.668 (0.7); 2.526 (2.1); 2.521 (3.3); 2.512 (54.3); 2.508 (113.7); 2.503
(152.7); 2.499 (112.6); 2.494 (55.9); 2.339 (0.4); 2.335 (0.8); 2.330 (1.1); 2.325 (0.8); 2.093 (16.0); 1.976 (0.4); 1.910 (0.5); 1.234
(1.6); 0.146 (0.8); 0.008 (5.6); 0.000 (178.1); −0.009 (6.9); −0.021 (0.4); −0.150 (0.8)
Compound No. I-1-344: ¹H NMR (400.0 MHz, d₆-DMSO):
12.461 (2.5); 8.865 (2.5); 8.860 (2.5); 8.803 (8.0); 8.791 (8.2); 8.625 (1.4); 8.615 (1.3); 8.134 (1.3); 8.094 (1.4); 8.090 (1.0); 8.078
(1.1); 8.074 (1.5); 7.606 (0.3); 7.597 (0.4); 7.467 (1.8); 7.455 (3.5); 7.449 (1.6); 7.442 (2.1); 7.437 (1.4); 7.429 (1.3); 7.417
(1.2); 7.109 (5.1); 7.009 (0.4); 7.002 (0.4); 6.987 (0.4); 6.980 (0.4); 5.755 (13.3); 5.498 (9.9); 3.822 (0.9); 3.810 (0.6); 3.798 (1.9);
3.790 (2.6); 2.524 (0.6); 2.510 (15.4); 2.506 (30.8); 2.502 (40.0); 2.497 (28.9); 2.493 (14.1); 2.346 (16.0); 2.328 (0.4); 1.755
(1.1); 0.008 (1.0); 0.000 (28.3); −0.009 (1.0)
Compound No. I-1-345: ¹H NMR (400.0 MHz, d₆-DMSO):
11.372 (3.5); 10.925 (1.0); 9.108 (1.3); 9.104 (1.3); 9.061 (3.0); 9.059 (3.2); 9.055 (3.3); 9.053 (3.0); 8.996 (0.5); 8.983 (11.9);
8.971 (11.9); 8.726 (3.3); 8.722 (3.5); 8.714 (3.5); 8.710 (3.4); 8.324 (0.5); 8.319 (0.8); 8.315 (1.0); 8.304 (0.6); 8.299 (0.8); 8.295
(0.6); 8.287 (1.5); 8.282 (1.9); 8.277 (1.4); 8.267 (1.6); 8.262 (2.0); 8.261 (2.0); 8.257 (1.5); 7.722 (3.2); 7.710 (6.1); 7.697
(3.1); 7.531 (0.8); 7.530 (0.8); 7.518 (0.8); 7.510 (2.5); 7.498 (2.4); 7.490 (1.8); 7.488 (1.7); 7.478 (1.7); 7.476 (1.7); 7.047 (4.4);
7.045 (4.3); 6.410 (0.6); 5.756 (2.0); 3.911 (1.3); 3.324 (4.8); 2.709 (16.0); 2.707 (15.7); 2.680 (0.5); 2.675 (0.9); 2.671 (1.2);
2.666 (0.9); 2.662 (0.4); 2.541 (0.9); 2.524 (3.1); 2.519 (4.7); 2.511 (64.0); 2.506 (131.2); 2.502 (172.7); 2.497 (122.4); 2.493
(57.0); 2.337 (0.3); 2.333 (0.8); 2.328 (1.1); 2.324 (0.8); 2.235 (7.9); 1.909 (1.7); 1.298 (2.1); 1.259 (3.1); 1.235 (2.5); 1.187 (0.4);
1.175 (0.4); 0.854 (0.4); 0.146 (0.3); 0.008 (2.9); 0.000 (88.2); −0.009 (2.8); −0.150 (0.3)
Compound No. I-1-346: ¹H NMR (400.0 MHz, d₆-DMSO):
8.744 (9.7); 8.732 (9.8); 8.464 (3.0); 8.317 (0.9); 7.630 (0.9); 7.430 (3.0); 7.418 (5.7); 7.406 (2.9); 7.248 (1.2); 5.902 (0.4); 5.889
(0.3); 5.335 (8.5); 3.326 (169.8); 3.294 (1.0); 3.269 (34.2); 2.675 (1.1); 2.671 (1.4); 2.666 (1.0); 2.541 (0.7); 2.506 (167.9); 2.502
(204.8); 2.497 (151.1); 2.333 (1.1); 2.329 (1.4); 2.324 (1.0); 2.184 (16.0); 0.146 (0.8); 0.000 (177.1); −0.008 (10.4); −0.150
(0.9)
Compound No. I-1-347: ¹H NMR (400.0 MHz, d₆-DMSO):
10.893 (0.7); 9.099 (0.7); 9.095 (0.7); 8.714 (0.5); 8.710 (0.5); 8.702 (0.5); 8.698 (0.5); 8.316 (0.4); 8.296 (0.4); 7.517 (0.4); 7.515
(0.4); 7.503 (0.4); 7.497 (0.4); 7.496 (0.4); 7.485 (0.4); 7.483 (0.4); 6.461 (1.1); 5.756 (0.3); 3.325 (7.5); 2.511 (3.3); 2.507
(6.7); 2.502 (8.9); 2.498 (6.6); 2.424 (4.4); 1.575 (16.0); 0.000 (0.9)
Compound No. I-1-348: ¹H NMR(601.6 MHz, CD3CN):
8.896 (0.6); 8.625 (0.7); 8.621 (0.7); 6.468 (0.8); 2.446 (4.5); 2.445 (4.5); 1.956 (0.4); 1.952 (0.5); 1.948 (2.8); 1.944 (4.9); 1.940
(7.0); 1.935 (4.6); 1.931 (2.3); 1.621 (0.5); 1.586 (16.0); 1.493 (0.3); 1.250 (0.5); 0.000 (1.6)
Compound No. I-1-349: ¹H NMR (400.0 MHz, d₆-DMSO):
11.237 (3.0); 9.150 (2.7); 9.145 (2.9); 8.747 (1.9); 8.744 (2.1); 8.735 (2.0); 8.732 (2.0); 8.367 (1.0); 8.362 (1.6); 8.357 (1.1); 8.347
(1.1); 8.342 (1.7); 8.337 (1.1); 7.575 (0.8); 7.569 (1.5); 7.564 (0.8); 7.552 (8.4); 7.549 (10.8); 7.541 (2.3); 7.532 (5.9); 7.520
(1.7); 7.511 (1.4); 7.434 (0.8); 7.430 (1.3); 7.424 (0.8); 7.413 (1.6); 7.401 (0.5); 7.397 (0.7); 7.392 (0.5); 6.760 (4.3); 3.326 (9.4);
2.507 (18.5); 2.503 (24.3); 2.499 (18.2); 2.373 (16.0); 0.008 (0.7); 0.000 (16.0)
Compound No. I-1-350: ¹H NMR (400.0 MHz, d₆-DMSO):
11.343 (3.0); 9.038 (2.1); 9.034 (3.5); 8.783 (3.3); 8.776 (3.4); 8.288 (1.1); 8.284 (1.3); 8.282 (1.4); 8.277 (1.1); 8.264 (1.1); 8.260
(1.3); 8.258 (1.3); 8.253 (1.0); 7.575 (0.7); 7.570 (1.3); 7.564 (0.7); 7.553 (9.2); 7.551 (9.6); 7.534 (4.6); 7.529 (1.3); 7.518
(0.7); 7.513 (1.3); 7.439 (0.8); 7.434 (1.4); 7.428 (0.8); 7.424 (0.9); 7.418 (1.5); 7.414 (0.9); 7.412 (0.7); 7.406 (0.5); 7.402 (0.8);
7.396 (0.5); 6.759 (4.3); 3.325 (16.1); 2.525 (0.7); 2.512 (12.3); 2.507 (24.3); 2.503 (31.7); 2.498 (22.9); 2.494 (11.0); 2.375
(16.0); 1.989 (0.4); 1.397 (1.2); 0.000 (1.2)
Compound No. I-1-351: mass (m/z): 368.1 (M + H)⁺; ¹H NMR (DMSO-d6) 2.40 (s, 3H), 5.35 (s, 2H), 5.76 (s, 1H),
6.50 (s, 1H), 7.36 (d, 1H), 7.50 (dd, 1H), 8.17 (d, 1H), 8.21 (s, 1H), 8.30 (m, 1H), 8.70 (dd, 1H), 9.09 (m, 1H),
11.00 (s, 1H).
Compound No. I-1-352: ¹H NMR (400.0 MHz, d₆-DMSO):
11.068 (3.3); 10.525 (0.7); 9.111 (2.6); 9.107 (2.7); 9.034 (0.6); 8.769 (0.4); 8.765 (0.4); 8.757 (0.4); 8.753 (0.4); 8.725 (1.8); 8.722
(2.0); 8.713 (2.0); 8.710 (2.0); 8.326 (1.6); 8.321 (1.1); 8.310 (1.1); 8.306 (1.7); 8.301 (1.1); 8.239 (0.3); 8.219 (0.4); 7.569
(0.3); 7.556 (0.4); 7.549 (0.3); 7.536 (0.4); 7.528 (1.6); 7.516 (1.6); 7.509 (1.6); 7.496 (1.5); 6.977 (0.7); 6.576 (4.4); 6.233 (0.9);
5.791 (5.7); 5.759 (1.2); 5.180 (1.8); 5.152 (8.2); 3.324 (98.6); 3.088 (1.7); 3.069 (5.6); 3.051 (6.2); 3.032 (2.7); 3.015 (0.6); 2.891
(1.6); 2.804 (1.0); 2.785 (1.0); 2.766 (0.3); 2.732 (1.4); 2.671 (1.1); 2.667 (0.8); 2.524 (2.4); 2.506 (127.8); 2.502 (169.3);
2.498 (126.0); 2.333 (0.9); 2.329 (1.2); 2.324 (1.0); 2.298 (16.0); 2.274 (0.5); 2.244 (0.4); 2.197 (0.6); 2.184 (3.8); 2.160 (0.7);
1.989 (0.4); 1.473 (0.4); 1.351 (0.5); 1.331 (6.2); 1.312 (12.7); 1.298 (4.0); 1.294 (6.5); 1.279 (1.8); 1.235 (15.5); 1.224 (2.2); 1.205
(2.3); 1.193 (0.4); 1.186 (1.1); 0.871 (0.6); 0.854 (1.6); 0.837 (0.7); 0.146 (1.7); 0.008 (12.3); 0.000 (329.7); −0.008
(14.8); −0.021 (0.9); −0.150 (1.7)
Compound No. I-1-353: ¹H NMR (400.0 MHz, d₆-DMSO):
11.051 (3.2); 10.626 (0.8); 9.092 (2.8); 9.088 (2.8); 9.038 (0.7); 9.034 (0.7); 8.775 (0.6); 8.771 (0.6); 8.763 (0.6); 8.758 (0.6); 8.715
(2.1); 8.711 (2.3); 8.703 (2.3); 8.699 (2.2); 8.310 (1.1); 8.306 (1.6); 8.301 (1.1); 8.290 (1.2); 8.286 (1.7); 8.281 (1.1); 8.240
(0.4); 8.219 (0.4); 7.596 (3.2); 7.575 (3.9); 7.565 (0.5); 7.555 (0.6); 7.546 (4.0); 7.544 (3.9); 7.527 (1.1); 7.515 (1.6); 7.513 (1.5);
7.503 (1.6); 7.501 (1.5); 7.495 (1.7); 7.493 (1.8); 7.483 (1.9); 7.229 (1.9); 7.227 (1.9); 7.206 (2.2); 7.185 (0.5); 7.182 (0.5); 6.575
(4.4); 6.218 (1.1); 5.757 (6.6); 5.592 (10.9); 3.328 (22.5); 2.525 (0.9); 2.512 (17.1); 2.507 (33.9); 2.503 (44.4); 2.498 (32.4);
2.494 (16.0); 2.415 (15.7); 2.401 (5.2); 2.393 (16.0); 2.330 (0.3); 2.155 (4.6); 1.234 (0.6); 0.146 (0.5); 0.008 (4.3); 0.000 (97.7);
−0.009 (4.5); −0.018 (0.4); −0.150 (0.5)
Compound No. I-2-2: ¹H NMR (600.1 MHz, d₆-DMSO):
10.894 (2.9); 9.067 (2.5); 9.064 (2.4); 8.825 (6.1); 8.816 (6.1); 8.803 (0.5); 8.795 (0.4); 8.786 (0.4); 8.750 (1.8); 8.748 (1.9); 8.742
(1.9); 8.740 (1.8); 8.273 (1.4); 8.259 (1.4); 7.549 (1.4); 7.541 (1.4); 7.536 (1.4); 7.528 (1.3); 7.497 (1.6); 7.489 (2.9); 7.481
(1.6); 7.263 (4.7); 7.178 (4.9); 7.093 (4.7); 5.581 (8.7); 5.516 (0.5); 5.320 (0.5); 3.372 (3.6); 3.174 (0.4); 2.845 (0.4); 2.692 (0.5);
2.632 (0.8); 2.514 (14.6); 2.512 (18.6); 2.509 (14.7); 2.452 (16.0); 2.332 (0.8); 2.287 (1.0)
Compound No. I-2-3: ¹H NMR (400.0 MHz, d₆-DMSO):
11.469 (4.7); 9.118 (3.4); 9.113 (3.4); 8.948 (9.2); 8.778 (2.4); 8.775 (2.6); 8.766 (2.6); 8.762 (2.5); 8.650 (14.2); 8.333 (1.3); 8.328
(2.0); 8.323 (1.3); 8.313 (1.5); 8.308 (2.1); 8.303 (1.3); 7.577 (1.9); 7.565 (1.9); 7.558 (1.8); 7.546 (1.7); 4.055 (1.2); 4.038
(3.7); 4.020 (3.8); 4.002 (1.3); 3.324 (73.0); 2.675 (0.6); 2.671 (0.8); 2.667 (0.6); 2.541 (0.6); 2.506 (93.1); 2.502 (120.4); 2.497
(87.6); 2.333 (0.6); 2.329 (0.8); 2.324 (0.6); 1.989 (16.0); 1.298 (0.3); 1.259 (0.5); 1.235 (0.7); 1.193 (4.3); 1.175 (8.4); 1.157
(4.2); 0.146 (0.9); 0.008 (8.3); 0.000 (189.2); −0.008 (7.8); −0.150 (0.9)
Compound No. I-2-4: ¹H NMR (400.0 MHz, d₆-DMSO):
10.968 (2.3); 9.063 (2.0); 9.059 (2.0); 8.747 (1.4); 8.744 (1.5); 8.735 (1.5); 8.732 (1.5); 8.581 (5.2); 8.310 (1.4); 8.303 (1.5); 8.287
(1.6); 8.280 (1.8); 8.274 (1.1); 8.270 (1.3); 8.250 (1.3); 8.147 (2.7); 8.140 (2.5); 8.081 (0.4); 7.543 (1.1); 7.531 (1.1); 7.523
(1.1); 7.512 (1.0); 7.303 (2.7); 7.280 (2.6); 7.271 (0.3); 5.757 (2.2); 5.442 (7.1); 5.173 (0.6); 3.976 (16.0); 3.962 (1.8); 3.898 (0.3);
3.327 (5.1); 2.525 (0.8); 2.511 (21.0); 2.507 (40.6); 2.503 (51.8); 2.498 (37.7); 2.494 (18.7); 2.329 (0.3); 0.000 (22.0); −0.008
(0.9)
Compound No. I-2-5: ¹H NMR (400.0 MHz, d₆-DMSO):
11.257 (1.4); 9.264 (3.4); 9.139 (1.2); 9.134 (1.2); 8.788 (0.8); 8.784 (0.9); 8.776 (0.9); 8.772 (0.9); 8.352 (0.4); 8.346 (0.7); 8.342
(0.5); 8.332 (0.5); 8.326 (0.7); 8.322 (0.5); 7.878 (1.2); 7.876 (1.6); 7.857 (1.8); 7.855 (1.7); 7.603 (1.1); 7.598 (0.5); 7.589
(1.0); 7.584 (1.8); 7.568 (0.9); 7.563 (1.3); 7.557 (0.8); 7.447 (0.7); 7.428 (1.0); 7.410 (0.4); 7.279 (0.3); 6.683 (0.3); 3.339 (28.7);
2.512 (5.3); 2.508 (10.7); 2.504 (14.2); 2.499 (10.5); 2.495 (5.3); 1.756 (16.0); 0.008 (0.8); 0.000 (20.3); −0.008 (0.9)
Compound No. I-2-6: ¹H NMR (400.0 MHz, d₆-DMSO):
11.141 (3.4); 9.121 (2.6); 9.116 (2.5); 8.774 (1.8); 8.770 (1.9); 8.762 (1.9); 8.758 (1.8); 8.334 (1.1); 8.329 (1.6); 8.325 (1.1); 8.314
(1.2); 8.309 (1.7); 8.305 (1.1); 7.601 (9.4); 7.590 (16.0); 7.578 (2.2); 7.566 (1.7); 7.558 (1.9); 7.547 (2.4); 7.537 (1.7); 7.526
(1.8); 7.519 (0.6); 7.514 (1.0); 7.505 (0.6); 7.489 (0.4); 7.481 (0.4); 7.476 (0.5); 4.056 (0.4); 4.038 (1.1); 4.020 (1.1); 4.002 (0.4);
3.357 (7.4); 2.847 (1.6); 2.828 (5.3); 2.809 (5.4); 2.790 (1.7); 2.694 (0.4); 2.676 (0.6); 2.512 (14.5); 2.507 (28.9); 2.503 (37.5);
2.498 (27.0); 2.494 (12.9); 1.989 (4.8); 1.258 (5.5); 1.240 (11.4); 1.221 (5.3); 1.193 (1.7); 1.175 (3.4); 1.157 (1.6); 0.008 (0.5);
0.000 (14.6); −0.009 (0.5)
Compound No. I-2-7: ¹H NMR (400.0 MHz, d₆-DMSO):
10.991 (5.0); 9.070 (4.2); 9.065 (4.2); 8.752 (2.9); 8.748 (3.3); 8.740 (3.1); 8.736 (3.2); 8.625 (9.9); 8.276 (2.4); 8.271 (1.7); 8.260
(1.7); 8.256 (2.5); 8.251 (1.7); 7.547 (2.5); 7.535 (2.7); 7.528 (4.5); 7.523 (2.3); 7.520 (2.3); 7.515 (3.7); 7.510 (2.2); 7.505
(3.5); 7.497 (0.5); 7.423 (0.6); 7.417 (0.9); 7.405 (3.1); 7.399 (5.1); 7.391 (4.9); 7.381 (5.3); 7.377 (3.9); 7.364 (1.0); 7.359 (0.6);
7.303 (3.0); 7.299 (1.9); 7.294 (1.5); 7.288 (1.8); 7.280 (1.9); 5.501 (16.0); 3.333 (27.4); 2.507 (24.7); 2.503 (32.6); 2.499 (24.7);
0.000 (1.8)
Compound No. I-2-8: ¹H NMR (400.0 MHz, d₆-DMSO):
10.909 (4.7); 9.050 (3.7); 9.029 (0.4); 8.771 (0.7); 8.761 (0.7); 8.739 (2.8); 8.730 (2.6); 8.658 (0.6); 8.634 (8.7); 8.316 (0.6); 8.258
(2.3); 8.238 (2.3); 8.092 (0.5); 7.925 (0.5); 7.647 (0.3); 7.614 (0.4); 7.595 (0.7); 7.569 (6.6); 7.550 (12.0); 7.535 (3.4); 7.523
(3.0); 7.516 (2.7); 7.503 (2.3); 7.474 (4.1); 7.455 (3.4); 7.452 (3.1); 7.433 (2.1); 5.894 (0.5); 5.794 (0.4); 5.756 (0.4); 5.624 (1.9);
5.614 (16.0); 5.362 (1.1); 5.290 (0.5); 3.332 (314.1); 2.671 (1.4); 2.667 (1.1); 2.507 (167.3); 2.502 (217.8); 2.498 (164.2); 2.329
(1.4); 2.075 (0.8); 1.234 (0.5); 0.008 (0.4); 0.000 (11.5)
Compound No. I-2-9: ¹H NMR (400.0 MHz, d₆-DMSO):
9.173 (0.5); 9.109 (1.3); 9.104 (1.3); 8.817 (0.8); 8.813 (0.9); 8.805 (0.9); 8.801 (0.9); 8.617 (0.3); 8.330 (0.5); 8.326 (0.8); 8.321
(0.7); 8.311 (0.8); 8.306 (0.9); 8.301 (0.7); 8.098 (2.3); 7.640 (0.7); 7.604 (0.6); 7.591 (0.7); 7.584 (0.7); 7.572 (0.6); 7.279 (0.4);
6.680 (0.4); 3.333 (39.9); 3.148 (0.5); 3.127 (2.1); 3.049 (5.8); 3.010 (6.2); 2.849 (0.5); 2.796 (2.0); 2.541 (0.6); 2.507 (27.3);
2.503 (35.0); 2.498 (25.3); 1.755 (16.0); 1.235 (0.9); 0.008 (1.7); 0.000 (40.3); −0.009 (1.7)
Compound No. I-2-10: ¹H NMR (400.0 MHz, d₆-DMSO):
11.523 (2.3); 9.519 (5.0); 9.452 (0.9); 9.140 (1.9); 8.796 (1.3); 8.787 (1.3); 8.359 (0.8); 8.354 (1.3); 8.350 (0.9); 8.339 (0.9); 8.334
(1.3); 8.330 (0.9); 7.598 (1.1); 7.586 (1.1); 7.578 (1.1); 7.566 (1.0); 4.549 (2.2); 4.532 (7.1); 4.514 (7.4); 4.496 (2.6); 4.492
(2.5); 4.474 (0.7); 4.112 (0.6); 4.098 (0.6); 3.336 (50.0); 3.182 (2.8); 3.169 (2.7); 2.549 (0.5); 2.514 (36.8); 2.510 (47.2); 2.506
(35.8); 1.402 (7.4); 1.384 (16.0); 1.367 (9.1); 1.352 (2.4); 1.224 (0.4)
Compound No. I-2-11: ¹H NMR (600.1 MHz, d₆-DMSO):
11.410 (0.9); 9.451 (5.5); 9.148 (1.8); 9.145 (1.8); 8.792 (1.3); 8.789 (1.3); 8.784 (1.3); 8.781 (1.3); 8.620 (3.1); 8.611 (3.1); 8.357
(0.7); 8.354 (1.1); 8.351 (0.8); 8.344 (0.8); 8.341 (1.1); 8.337 (0.8); 7.590 (1.0); 7.589 (1.0); 7.582 (1.0); 7.581 (1.0); 7.577
(1.0); 7.576 (1.0); 7.569 (1.0); 7.568 (0.9); 7.008 (3.2); 6.999 (3.2); 4.061 (16.0); 3.348 (64.6); 2.513 (3.8); 2.510 (8.0); 2.507 (10.9);
2.504 (7.9); 2.501 (3.8); 1.761 (7.0); 0.000 (1.3)
Compound No. I-2-12: ¹H NMR (400.0 MHz, d₆-DMSO):
10.993 (2.0); 9.086 (1.2); 8.763 (0.8); 8.753 (0.8); 8.299 (0.7); 8.295 (1.0); 8.290 (0.7); 8.280 (0.8); 8.275 (1.1); 8.270 (0.7); 7.566
(1.0); 7.554 (1.0); 7.546 (1.0); 7.534 (0.9); 4.073 (1.1); 4.055 (3.4); 4.037 (3.4); 4.019 (1.1); 3.826 (0.4); 3.808 (0.4); 3.368
(1.1); 3.169 (3.1); 2.653 (16.0); 2.547 (0.4); 2.511 (8.5); 2.507 (16.6); 2.503 (21.5); 2.498 (15.5); 2.494 (7.5); 2.394 (2.1); 1.353
(3.5); 1.335 (7.4); 1.317 (3.4); 1.211 (0.5); 1.193 (1.0); 1.175 (0.5); 0.007 (1.3); 0.000 (28.5); −0.008 (1.1)
Compound No. I-2-13: ¹H NMR (600.1 MHz, d₆-DMSO):
10.979 (3.9); 9.070 (4.1); 9.067 (4.1); 8.751 (3.5); 8.749 (3.7); 8.743 (3.7); 8.741 (3.6); 8.623 (11.9); 8.272 (2.3); 8.269 (1.7); 8.262
(1.8); 8.259 (2.4); 7.545 (2.5); 7.544 (2.5); 7.537 (2.5); 7.536 (2.5); 7.532 (2.5); 7.531 (2.4); 7.524 (2.4); 7.523 (2.3); 7.469
(1.1); 7.465 (8.2); 7.462 (3.0); 7.454 (3.6); 7.451 (10.9); 7.447 (1.6); 7.365 (9.0); 7.351 (6.8); 5.395 (16.0); 3.337 (36.9); 2.525
(0.3); 2.522 (0.4); 2.519 (0.5); 2.510 (8.5); 2.507 (17.2); 2.504 (23.1); 2.501 (16.8); 2.498 (8.1); 1.759 (7.2); 0.000 (5.8)
Compound No. I-2-14: ¹H NMR (400.0 MHz, d₆-DMSO):
9.178 (0.4); 8.370 (0.4); 8.350 (0.4); 7.277 (0.4); 6.680 (0.4); 3.713 (7.7); 3.330 (11.8); 2.507 (19.5); 2.502 (24.7); 2.498 (17.6);
1.754 (16.0); 1.073 (0.3); 1.055 (0.7); 1.038 (0.3); 0.000 (2.2)
Compound No. I-2-15: ¹H NMR (400.0 MHz, d₆-DMSO):
11.305 (7.2); 11.201 (0.4); 9.130 (6.2); 9.126 (6.1); 9.074 (0.8); 9.071 (0.8); 9.040 (16.0); 8.964 (0.6); 8.913 (1.1); 8.799 (0.7);
8.795 (0.8); 8.783 (5.1); 8.779 (4.9); 8.771 (4.8); 8.767 (4.8); 8.680 (0.9); 8.660 (5.1); 8.656 (5.5); 8.648 (5.3); 8.645 (5.3); 8.628
(0.4); 8.625 (0.4); 8.617 (0.4); 8.613 (0.4); 8.567 (0.4); 8.563 (0.4); 8.554 (2.6); 8.547 (0.9); 8.544 (0.9); 8.536 (0.8); 8.532 (0.8);
8.466 (5.2); 8.462 (5.1); 8.445 (5.6); 8.442 (5.3); 8.435 (0.6); 8.419 (0.5); 8.415 (0.5); 8.367 (0.4); 8.363 (0.4); 8.344 (3.3); 8.340
(3.8); 8.335 (2.6); 8.325 (3.3); 8.320 (4.0); 8.315 (2.7); 8.284 (0.4); 8.279 (0.6); 8.274 (0.4); 8.264 (0.4); 8.259 (0.6); 8.254
(0.4); 8.208 (0.4); 8.069 (0.3); 7.627 (5.9); 7.615 (5.4); 7.607 (5.6); 7.600 (0.8); 7.595 (5.3); 7.583 (3.7); 7.571 (3.7); 7.563 (3.8);
7.551 (3.7); 7.534 (0.5); 7.497 (0.7); 7.486 (0.7); 7.475 (1.0); 7.463 (1.0); 7.454 (0.9); 7.443 (0.8); 7.202 (0.3); 7.074 (0.3); 6.946
(0.5); 5.687 (1.0); 3.328 (116.4); 2.676 (0.7); 2.671 (1.0); 2.667 (0.8); 2.566 (6.8); 2.542 (30.2); 2.525 (2.9); 2.511 (58.4); 2.507
(116.7); 2.502 (153.1); 2.498 (110.7); 2.493 (53.3); 2.338 (0.4); 2.333 (0.7); 2.329 (1.0); 2.325 (0.7); 0.146 (0.6); 0.008 (5.6);
0.000 (149.6); −0.009 (5.6); −0.150 (0.7)
Compound No. I-2-16: ¹H NMR (400.0 MHz, d₆-DMSO):
10.846 (1.9); 9.070 (1.9); 9.066 (2.0); 8.757 (1.2); 8.753 (1.3); 8.745 (1.3); 8.741 (1.2); 8.282 (0.7); 8.277 (1.2); 8.272 (0.8); 8.262
(0.8); 8.257 (1.2); 8.252 (0.9); 7.556 (1.1); 7.544 (1.1); 7.536 (1.1); 7.524 (1.0); 4.411 (0.4); 4.038 (0.8); 4.029 (16.0); 3.902
(0.6); 3.813 (0.7); 3.805 (0.9); 3.689 (0.4); 3.556 (0.6); 3.541 (15.0); 3.354 (0.8); 3.334 (20.4); 3.290 (0.6); 2.508 (18.4); 2.503
(23.9); 2.499 (17.4); 2.415 (0.7); 0.000 (5.7)
Compound No. I-2-17: ¹H NMR (400.0 MHz, d₆-DMSO):
10.851 (1.9); 9.080 (1.2); 8.755 (0.9); 8.745 (0.9); 8.293 (0.8); 8.288 (1.1); 8.283 (0.8); 8.273 (0.8); 8.268 (1.2); 8.263 (0.8); 7.558
(1.0); 7.546 (1.1); 7.538 (1.0); 7.526 (0.9); 3.757 (16.0); 3.558 (2.8); 3.349 (2.2); 3.169 (0.3); 2.780 (1.1); 2.761 (3.7); 2.742
(3.8); 2.723 (1.3); 2.643 (0.5); 2.624 (0.5); 2.512 (8.5); 2.508 (16.8); 2.503 (21.8); 2.499 (15.7); 2.494 (7.7); 1.255 (4.1); 1.236
(8.5); 1.217 (4.0); 1.186 (0.7); 1.168 (1.4); 1.149 (0.6); 0.000 (5.2)
Compound No. I-2-18: ¹H NMR (400.0 MHz, d₆-DMSO):
11.080 (3.6); 9.100 (3.5); 9.096 (3.6); 8.770 (2.3); 8.767 (2.6); 8.758 (2.5); 8.755 (2.6); 8.677 (7.5); 8.311 (1.3); 8.307 (2.0); 8.303
(1.5); 8.292 (1.4); 8.287 (2.1); 7.569 (1.9); 7.557 (1.9); 7.550 (1.9); 7.538 (1.8); 5.450 (16.0); 3.334 (17.1); 3.310 (28.7); 2.508
(17.9); 2.504 (23.5); 2.500 (18.3); 1.756 (4.5); 0.000 (4.9)
Compound No. I-2-19: ¹H NMR (400.0 MHz, d₆-DMSO):
10.989 (2.3); 9.069 (1.9); 9.065 (2.0); 8.753 (1.4); 8.749 (1.5); 8.741 (1.5); 8.737 (1.5); 8.640 (4.8); 8.276 (1.1); 8.256 (1.2); 7.549
(1.1); 7.537 (1.1); 7.529 (1.1); 7.517 (1.0); 7.449 (0.3); 7.427 (1.8); 7.411 (7.9); 7.399 (0.6); 7.309 (1.1); 7.305 (1.6); 7.299
(1.1); 7.295 (1.0); 7.289 (1.3); 6.677 (0.4); 5.410 (6.8); 3.331 (84.4); 2.671 (0.4); 2.507 (45.7); 2.502 (59.0); 2.498 (44.4); 2.329
(0.4); 1.989 (1.1); 1.754 (16.0); 1.175 (0.6); 0.000 (10.9)
Compound No. I-2-20: ¹H NMR (400.0 MHz, d₆-DMSO):
10.807 (2.1); 9.075 (1.9); 9.071 (2.0); 8.753 (1.4); 8.749 (1.5); 8.741 (1.5); 8.737 (1.5); 8.284 (0.8); 8.279 (1.2); 8.275 (0.8); 8.264
(0.9); 8.259 (1.2); 8.255 (0.8); 7.553 (1.2); 7.542 (1.2); 7.534 (1.2); 7.522 (1.1); 4.620 (0.4); 4.604 (1.0); 4.587 (1.4); 4.571
(1.1); 4.554 (0.4); 3.332 (1.6); 2.507 (16.4); 2.502 (21.2); 2.498 (15.4); 2.411 (16.0); 2.234 (1.2); 1.390 (13.8); 1.374 (13.6); 1.293
(1.6); 1.276 (1.6); 0.008 (0.7); 0.000 (16.6); −0.008 (0.6)
Compound No. I-2-21: ¹H NMR (400.0 MHz, d₆-DMSO):
10.941 (3.6); 9.084 (2.8); 8.759 (2.1); 8.750 (2.1); 8.456 (5.9); 8.299 (1.3); 8.295 (2.0); 8.290 (1.4); 8.280 (1.7); 8.275 (2.3); 8.270
(1.4); 8.261 (0.5); 7.563 (2.0); 7.551 (2.1); 7.544 (2.0); 7.532 (1.9); 4.122 (4.2); 4.104 (8.5); 4.087 (4.3); 3.873 (0.3); 3.856
(0.7); 3.839 (0.4); 3.347 (7.6); 2.672 (0.4); 2.525 (1.0); 2.512 (22.9); 2.507 (45.9); 2.503 (60.1); 2.498 (43.6); 2.494 (21.4); 2.329
(0.4); 1.852 (0.5); 1.834 (2.4); 1.816 (4.8); 1.798 (4.9); 1.780 (2.6); 1.762 (0.6); 1.719 (0.4); 1.701 (0.4); 0.870 (7.7); 0.852 (16.0);
0.833 (7.3); 0.821 (0.9); 0.803 (1.3); 0.784 (0.6); 0.008 (0.5); 0.000 (14.1); −0.008 (0.6)
Compound No. I-2-22: ¹H NMR (400.0 MHz, d₆-DMSO):
11.396 (7.8); 9.343 (16.0); 9.264 (0.3); 9.146 (6.2); 9.141 (6.2); 8.793 (4.4); 8.789 (4.7); 8.781 (4.6); 8.777 (4.5); 8.358 (2.5); 8.353
(3.7); 8.348 (2.4); 8.338 (2.7); 8.333 (3.9); 8.328 (2.5); 7.594 (3.4); 7.582 (3.4); 7.575 (3.4); 7.563 (3.2); 7.354 (11.7); 7.324
(0.4); 3.332 (47.1); 2.683 (0.6); 2.674 (0.4); 2.544 (6.0); 2.525 (80.0); 2.513 (20.1); 2.509 (37.7); 2.505 (48.9); 2.500 (37.3); 2.468
(0.5); 2.447 (1.0); 2.388 (0.4); 2.363 (0.5); 2.331 (0.3); 0.008 (1.7); 0.000 (39.3); −0.008 (1.5)
Compound No. I-2-23: ¹H NMR (400.0 MHz, d₆-DMSO):
10.954 (3.0); 9.070 (2.4); 9.065 (2.4); 8.750 (1.8); 8.746 (1.9); 8.738 (1.9); 8.734 (1.8); 8.564 (6.1); 8.276 (1.4); 8.271 (1.0); 8.260
(1.0); 8.256 (1.5); 7.547 (1.4); 7.535 (1.4); 7.527 (1.4); 7.515 (1.3); 7.245 (1.1); 7.242 (1.2); 7.227 (5.5); 7.215 (1.8); 7.209
(0.8); 7.196 (1.4); 7.189 (1.2); 7.182 (0.7); 7.174 (0.7); 7.126 (2.1); 7.108 (1.4); 5.397 (9.2); 3.324 (37.3); 2.675 (0.3); 2.671 (0.4);
2.541 (4.3); 2.506 (50.8); 2.502 (65.5); 2.497 (48.4); 2.347 (16.0); 2.329 (0.6); 2.303 (0.4); 0.007 (2.5); 0.000 (49.3); −0.008
(2.2)
Compound No. I-2-24: ¹H NMR (400.0 MHz, d₆-DMSO):
11.423 (1.9); 9.468 (2.7); 9.145 (1.5); 8.791 (1.0); 8.779 (1.1); 8.354 (0.9); 8.334 (0.9); 7.592 (0.8); 7.579 (0.8); 7.572 (0.8); 7.560
(0.7); 6.318 (3.0); 4.007 (16.0); 3.332 (11.2); 2.504 (20.9); 0.000 (5.3)
Compound No. I-2-25: ¹H NMR (400.0 MHz, d₆-DMSO):
11.367 (3.8); 9.307 (8.3); 9.142 (2.8); 9.137 (2.8); 8.790 (1.9); 8.787 (2.0); 8.778 (1.9); 8.775 (1.9); 8.354 (1.3); 8.349 (1.9); 8.344
(1.3); 8.334 (1.4); 8.329 (2.0); 8.324 (1.3); 7.592 (1.7); 7.580 (1.7); 7.572 (1.7); 7.560 (1.6); 3.333 (83.5); 2.672 (0.4); 2.533
(41.0); 2.511 (25.0); 2.507 (48.6); 2.503 (63.0); 2.499 (45.8); 2.330 (0.4); 2.263 (16.0); 1.755 (4.1); 0.007 (1.0); 0.000 (23.2); −0.008
(1.0)
Compound No. I-2-26: ¹H NMR (400.0 MHz, d₆-DMSO):
10.989 (2.0); 9.085 (1.9); 9.080 (1.9); 8.764 (1.3); 8.760 (1.4); 8.752 (1.4); 8.748 (1.4); 8.297 (0.8); 8.292 (1.1); 8.287 (0.8); 8.277
(0.9); 8.272 (1.2); 8.267 (0.8); 7.563 (1.1); 7.551 (1.1); 7.543 (1.1); 7.531 (1.0); 3.715 (14.9); 3.474 (5.1); 3.337 (16.8); 2.644
(16.0); 2.539 (5.4); 2.508 (19.7); 2.503 (25.8); 2.499 (19.1); 0.000 (5.3)
Compound No. I-2-27: ¹H NMR (400.0 MHz, d₆-DMSO):
11.607 (5.3); 9.013 (9.0); 9.009 (9.1); 8.799 (5.6); 8.795 (6.4); 8.787 (6.0); 8.783 (6.2); 8.208 (3.2); 8.204 (5.1); 8.199 (3.7); 8.189
(3.5); 8.184 (5.4); 8.179 (3.8); 7.614 (10.0); 7.594 (14.5); 7.579 (4.8); 7.567 (4.7); 7.559 (4.5); 7.547 (4.4); 7.530 (6.9); 7.512
(16.0); 7.492 (7.8); 7.459 (5.5); 7.441 (6.2); 7.423 (2.2); 7.411 (0.4); 6.818 (0.9); 3.327 (94.7); 2.996 (0.4); 2.712 (0.4); 2.671
(0.9); 2.542 (76.8); 2.506 (107.2); 2.502 (138.2); 2.498 (110.3); 2.368 (0.4); 2.329 (0.9); 0.146 (0.4); 0.000 (88.4); −0.150
(0.4)
Compound No. I-2-28: mass (m/z): 296.1 (M + H)⁺; ¹H NMR (DMSO-d6) 3.95 (s, 3H), 7.4-7.5 (m, 5H), 8.20 (m,
1H), 8.79 (m, 2H), 9.02 (m, 1H), 11.36 (s, 1H).
Compound No. I-3-2: ¹H NMR (400.0 MHz, d₆-DMSO):
11.504 (1.6); 9.148 (1.9); 9.144 (2.0); 8.772 (1.4); 8.768 (1.6); 8.760 (1.5); 8.756 (1.5); 8.361 (0.8); 8.357 (1.2); 8.352 (0.9); 8.342
(0.9); 8.336 (1.2); 8.332 (0.9); 8.003 (4.5); 7.577 (1.1); 7.576 (1.1); 7.565 (1.1); 7.563 (1.1); 7.557 (1.1); 7.556 (1.1); 7.545
(1.0); 7.544 (1.0); 4.117 (16.0); 3.325 (4.1); 2.512 (5.4); 2.508 (11.0); 2.503 (14.7); 2.499 (11.0); 2.495 (5.7); 0.008 (0.5); 0.000
(13.7); −0.008 (0.6)
Compound No. I-3-3: ¹H NMR (400.0 MHz, d₆-DMSO):
11.107 (8.3); 9.182 (9.3); 9.178 (9.3); 8.840 (6.8); 8.837 (7.1); 8.828 (7.2); 8.825 (6.9); 8.413 (0.4); 8.385 (3.5); 8.380 (5.1); 8.375
(3.5); 8.365 (3.9); 8.360 (5.4); 8.355 (3.5); 7.995 (9.8); 7.992 (12.8); 7.973 (13.5); 7.916 (0.4); 7.811 (0.4); 7.640 (5.3); 7.630
(11.1); 7.622 (5.9); 7.620 (6.1); 7.610 (16.0); 7.591 (9.2); 7.502 (5.2); 7.483 (7.6); 7.465 (3.0); 4.056 (0.5); 4.039 (1.6); 4.021
(1.6); 4.003 (0.5); 3.336 (114.0); 2.677 (0.5); 2.673 (0.6); 2.669 (0.4); 2.509 (71.7); 2.504 (91.4); 2.500 (65.4); 2.331 (0.6); 2.326
(0.4); 1.990 (6.9); 1.397 (0.9); 1.193 (1.9); 1.176 (3.7); 1.158 (1.8); 0.000 (2.1)
Compound No. I-3-4: ¹H NMR (400.0 MHz, d₆-DMSO):
11.888 (6.6); 9.199 (5.7); 9.195 (5.9); 8.801 (4.2); 8.797 (4.5); 8.789 (4.5); 8.785 (4.4); 8.434 (16.0); 8.414 (2.5); 8.410 (3.5); 8.405
(2.4); 8.395 (2.6); 8.390 (3.7); 8.385 (2.5); 8.318 (0.7); 8.009 (1.1); 8.001 (11.6); 7.996 (3.6); 7.984 (3.8); 7.979 (13.2); 7.971
(1.3); 7.675 (1.5); 7.668 (13.5); 7.662 (3.9); 7.650 (3.7); 7.645 (11.6); 7.638 (1.2); 7.608 (3.4); 7.596 (3.3); 7.588 (3.3); 7.576
(3.1); 7.536 (0.4); 7.473 (0.6); 4.020 (0.4); 3.363 (0.5); 3.330 (303.9); 3.309 (1.1); 2.676 (1.5); 2.672 (2.0); 2.667 (1.4); 2.525
(4.0); 2.512 (114.7); 2.507 (233.3); 2.503 (304.7); 2.498 (214.8); 2.494 (100.3); 2.334 (1.3); 2.329 (1.9); 2.325 (1.3); 1.989 (1.5);
1.259 (0.4); 1.232 (0.8); 1.193 (0.4); 1.175 (0.8); 1.157 (0.5); 0.146 (1.0); 0.008 (7.2); 0.000 (214.6); −0.009 (7.2); −0.018
(0.7); −0.022 (0.5); −0.150 (1.0)
Compound No. I-3-5: ¹H NMR (400.0 MHz, d₆-DMSO):
12.007 (5.7); 9.212 (5.5); 9.208 (5.6); 9.058 (5.3); 9.055 (5.2); 8.893 (5.5); 8.888 (5.2); 8.808 (4.0); 8.804 (4.2); 8.796 (4.2); 8.792
(4.1); 8.578 (16.0); 8.429 (2.3); 8.424 (3.3); 8.419 (2.2); 8.409 (2.5); 8.404 (3.4); 8.399 (2.3); 7.613 (3.0); 7.601 (2.9); 7.593
(2.9); 7.581 (2.8); 5.759 (2.1); 3.332 (48.8); 2.678 (0.4); 2.673 (0.5); 2.669 (0.3); 2.527 (1.2); 2.513 (28.6); 2.509 (57.6); 2.504
(74.8); 2.500 (52.8); 2.495 (24.7); 2.336 (0.3); 2.331 (0.4); 1.990 (1.1); 1.352 (0.5); 1.337 (0.7); 1.299 (2.8); 1.259 (4.1); 1.250
(1.3); 1.232 (2.8); 1.193 (0.5); 1.188 (0.5); 1.176 (0.8); 1.158 (0.4); 0.868 (0.4); 0.853 (0.5); 0.008 (2.1); 0.000 (58.6); −0.009
(1.9)
Compound No. I-3-6: ¹H NMR (400.0 MHz, d₆-DMSO):
11.472 (11.8); 9.181 (8.5); 9.176 (8.4); 8.847 (5.7); 8.844 (6.0); 8.835 (6.0); 8.832 (5.8); 8.702 (7.9); 8.698 (8.3); 8.690 (8.3); 8.687
(8.3); 8.447 (8.1); 8.443 (8.1); 8.427 (8.9); 8.423 (8.4); 8.388 (3.9); 8.383 (5.7); 8.378 (3.9); 8.368 (4.2); 8.363 (6.0); 8.358
(4.0); 8.319 (0.4); 7.858 (8.5); 7.847 (8.2); 7.838 (7.9); 7.826 (7.8); 7.643 (5.1); 7.631 (5.1); 7.623 (5.0); 7.612 (4.7); 7.611 (4.7);
5.760 (16.0); 4.057 (0.4); 4.039 (1.1); 4.022 (1.1); 4.004 (0.4); 3.335 (93.6); 2.679 (0.4); 2.674 (0.6); 2.670 (0.4); 2.527 (1.6);
2.514 (35.4); 2.510 (70.9); 2.505 (92.7); 2.501 (66.2); 2.497 (31.4); 2.467 (0.3); 2.336 (0.4); 2.332 (0.6); 2.327 (0.4); 1.991 (4.9);
1.338 (0.8); 1.300 (2.3); 1.260 (3.3); 1.250 (1.4); 1.234 (3.4); 1.194 (1.4); 1.189 (0.5); 1.176 (2.7); 1.159 (1.3); 0.853 (0.6); 0.840
(0.3); 0.835 (0.4); 0.008 (0.8); 0.000 (21.6); −0.008 (0.8)
Compound No. I-3-7: ¹H NMR (400.0 MHz, d₆-DMSO):
11.881 (5.0); 9.201 (5.1); 9.197 (5.1); 8.800 (3.6); 8.796 (3.8); 8.788 (3.9); 8.784 (3.7); 8.665 (4.1); 8.661 (4.3); 8.654 (4.4); 8.650
(4.2); 8.482 (4.2); 8.478 (4.2); 8.460 (16.0); 8.417 (2.1); 8.413 (3.0); 8.408 (2.1); 8.397 (2.3); 8.393 (3.0); 8.388 (2.1); 7.653
(4.2); 7.641 (4.0); 7.633 (4.0); 7.621 (4.0); 7.606 (2.7); 7.605 (2.6); 7.594 (2.7); 7.593 (2.6); 7.586 (2.7); 7.585 (2.5); 7.574 (2.5);
7.573 (2.4); 7.527 (0.4); 4.038 (0.8); 4.020 (0.8); 3.333 (67.0); 3.176 (1.0); 3.163 (0.9); 2.673 (0.4); 2.508 (48.8); 2.504 (62.6);
2.499 (45.0); 2.495 (22.0); 2.331 (0.4); 1.990 (3.4); 1.299 (0.5); 1.259 (0.7); 1.233 (0.5); 1.193 (1.0); 1.175 (1.9); 1.157 (0.9);
0.008 (1.0); 0.000 (24.3); −0.008 (1.1)
Compound No. I-3-8: ¹H NMR (400.0 MHz, d₆-DMSO):
11.458 (6.5); 9.170 (7.5); 9.165 (7.4); 8.840 (5.0); 8.837 (5.2); 8.828 (5.3); 8.825 (5.1); 8.378 (2.8); 8.373 (4.2); 8.368 (2.7); 8.358
(3.0); 8.353 (4.5); 8.348 (2.7); 8.066 (8.9); 8.061 (9.0); 7.961 (8.6); 7.939 (10.5); 7.757 (5.9); 7.752 (5.6); 7.736 (4.9); 7.730
(4.7); 7.636 (4.0); 7.624 (4.0); 7.616 (3.9); 7.604 (3.7); 4.121 (1.2); 4.108 (3.4); 4.095 (3.5); 4.082 (1.2); 3.334 (45.5); 3.177 (16.0);
3.164 (15.5); 2.678 (0.3); 2.674 (0.4); 2.669 (0.3); 2.509 (52.1); 2.505 (66.4); 2.501 (47.8); 2.331 (0.4); 1.234 (0.9); 0.008
(1.2); 0.000 (25.3); −0.008 (1.0)
Compound No. I-3-9: ¹H NMR (400.0 MHz, d₆-DMSO):
11.056 (2.4); 9.186 (2.1); 9.182 (2.1); 8.812 (1.5); 8.808 (1.6); 8.800 (1.6); 8.796 (1.6); 8.612 (1.8); 8.609 (1.8); 8.601 (1.8); 8.597
(1.8); 8.381 (1.2); 8.361 (1.3); 8.327 (1.7); 8.324 (1.7); 8.307 (1.9); 8.303 (1.8); 7.708 (1.8); 7.696 (1.8); 7.687 (1.7); 7.676
(1.6); 7.616 (1.2); 7.604 (1.3); 7.596 (1.2); 7.584 (1.1); 3.330 (48.2); 2.507 (23.4); 2.503 (29.7); 2.499 (22.2); 2.308 (16.0); 1.299
(0.4); 1.259 (0.6); 0.008 (0.5); 0.000 (11.1)
Compound No. I-3-10: ¹H NMR (400.0 MHz, d₆-DMSO):
11.565 (1.7); 9.167 (0.7); 8.774 (0.6); 8.385 (0.7); 8.381 (1.0); 8.376 (0.7); 8.365 (0.7); 8.361 (1.1); 8.356 (0.7); 8.023 (4.6); 7.585
(0.8); 7.573 (0.9); 7.565 (0.9); 7.553 (0.8); 4.804 (0.4); 4.787 (1.1); 4.770 (1.6); 4.754 (1.2); 4.737 (0.5); 3.523 (0.3); 2.514
(5.1); 2.509 (10.4); 2.505 (13.7); 2.500 (9.7); 2.496 (4.6); 1.507 (16.0); 1.491 (15.7); 0.000 (3.0)
Compound No. I-3-11: ¹H NMR (400.0 MHz, d₆-DMSO):
11.023 (2.2); 9.188 (1.8); 9.184 (1.9); 8.815 (1.4); 8.811 (1.5); 8.803 (1.6); 8.799 (1.6); 8.387 (0.7); 8.382 (1.1); 8.377 (0.7); 8.367
(0.8); 8.362 (1.2); 8.357 (0.7); 7.963 (2.2); 7.960 (2.9); 7.941 (3.0); 7.743 (0.3); 7.741 (0.4); 7.722 (0.4); 7.619 (1.1); 7.607
(1.1); 7.600 (1.1); 7.584 (2.2); 7.579 (0.7); 7.565 (3.2); 7.554 (0.4); 7.544 (2.3); 7.426 (0.5); 7.414 (1.1); 7.405 (0.4); 7.396 (1.8);
7.377 (0.8); 3.334 (9.7); 2.526 (0.4); 2.513 (8.4); 2.509 (17.0); 2.504 (22.2); 2.500 (15.7); 2.495 (7.3); 2.303 (16.0); 2.187 (2.5);
0.000 (4.5)
Compound No. I-3-12: ¹H NMR (400.0 MHz, d₆-DMSO):
11.411 (11.9); 9.176 (10.6); 9.172 (10.7); 9.171 (10.0); 8.845 (8.0); 8.841 (8.5); 8.833 (8.6); 8.828 (8.4); 8.384 (4.7); 8.379 (6.2);
8.374 (4.7); 8.364 (5.1); 8.358 (6.7); 8.354 (4.9); 8.317 (0.8); 8.067 (10.8); 8.063 (16.0); 8.059 (4.2); 8.048 (8.5); 8.045 (15.1);
8.042 (15.0); 8.036 (1.8); 8.004 (0.3); 7.675 (7.7); 7.670 (3.3); 7.656 (16.0); 7.653 (9.7); 7.640 (10.8); 7.636 (12.2); 7.630 (7.9);
7.628 (7.0); 7.622 (6.0); 7.620 (5.9); 7.610 (5.8); 7.608 (5.8); 7.583 (4.0); 7.580 (7.0); 7.578 (4.2); 7.566 (3.0); 7.562 (9.4);
7.557 (2.6); 7.546 (2.2); 7.543 (3.4); 7.541 (2.0); 3.327 (208.7); 2.681 (0.5); 2.677 (1.1); 2.672 (1.5); 2.667 (1.1); 2.663 (0.5); 2.542
(12.0); 2.525 (3.8); 2.521 (6.2); 2.512 (86.7); 2.507 (177.0); 2.503 (234.1); 2.498 (168.3); 2.494 (80.0); 2.339 (0.6); 2.334
(1.1); 2.330 (1.6); 2.325 (1.1); 2.104 (1.1); 0.146 (0.9); 0.008 (7.3); 0.000 (214.2); −0.009 (7.5); −0.016 (0.7); −0.150 (0.9)
Compound No. I-3-13: ¹H NMR (400.0 MHz, d₆-DMSO):
11.182 (11.9); 9.180 (8.6); 9.176 (8.6); 8.834 (7.0); 8.830 (7.5); 8.822 (7.5); 8.818 (7.4); 8.384 (3.5); 8.379 (5.0); 8.374 (3.5); 8.364
(3.8); 8.359 (5.3); 8.354 (3.5); 7.817 (6.5); 7.812 (6.3); 7.801 (7.2); 7.797 (16.0); 7.793 (8.7); 7.782 (7.7); 7.778 (8.8); 7.683
(1.9); 7.677 (4.0); 7.673 (4.4); 7.667 (1.6); 7.658 (9.4); 7.654 (6.8); 7.639 (6.0); 7.634 (9.7); 7.628 (7.7); 7.624 (7.6); 7.621 (6.5);
7.619 (7.0); 7.611 (7.6); 7.609 (9.0); 7.605 (8.5); 7.601 (6.7); 7.595 (3.1); 7.590 (3.1); 7.586 (2.8); 7.519 (0.6); 7.514 (0.4); 7.507
(3.8); 7.501 (2.3); 7.496 (2.5); 7.494 (2.5); 7.490 (2.1); 7.483 (3.4); 7.471 (0.4); 5.769 (4.7); 3.349 (0.4); 3.332 (80.0); 3.316
(0.4); 2.714 (0.6); 2.678 (0.3); 2.673 (0.5); 2.669 (0.4); 2.562 (0.6); 2.544 (154.2); 2.527 (1.6); 2.522 (2.2); 2.513 (28.3); 2.509
(57.5); 2.504 (75.9); 2.500 (54.7); 2.495 (26.2); 2.370 (0.6); 2.336 (0.4); 2.331 (0.5); 2.327 (0.4); 0.146 (0.3); 0.008 (2.7); 0.000
(74.6); −0.009 (2.6); −0.150 (0.3)
Compound No. I-3-14: ¹H NMR (400.0 MHz, d₆-DMSO):
12.080 (4.6); 9.223 (5.7); 9.219 (5.7); 8.997 (4.7); 8.996 (4.8); 8.993 (4.7); 8.812 (4.1); 8.808 (4.3); 8.800 (4.3); 8.796 (4.2); 8.580
(16.0); 8.492 (2.7); 8.486 (2.7); 8.470 (3.2); 8.465 (3.1); 8.438 (2.4); 8.433 (3.3); 8.428 (2.4); 8.418 (2.6); 8.413 (3.5); 8.408
(2.4); 8.317 (0.4); 8.196 (5.2); 8.175 (4.5); 7.618 (3.0); 7.616 (3.0); 7.606 (3.0); 7.604 (3.0); 7.598 (3.0); 7.596 (2.9); 7.586 (2.8);
7.584 (2.8); 3.326 (107.7); 2.677 (0.6); 2.672 (0.8); 2.668 (0.6); 2.543 (3.8); 2.525 (2.4); 2.512 (50.0); 2.508 (98.4); 2.503 (127.6);
2.499 (92.2); 2.494 (44.7); 2.334 (0.6); 2.330 (0.8); 2.326 (0.6); 0.146 (0.5); 0.008 (4.1); 0.000 (103.9); −0.009 (4.0); −0.150
(0.5)
Compound No. I-3-15: ¹H NMR (400.0 MHz, d₆-DMSO):
11.919 (5.6); 9.199 (6.1); 9.195 (6.0); 8.800 (4.3); 8.796 (4.5); 8.788 (4.6); 8.784 (4.4); 8.727 (7.8); 8.722 (8.9); 8.661 (8.8); 8.655
(7.5); 8.502 (16.0); 8.416 (2.6); 8.411 (3.6); 8.406 (2.5); 8.396 (2.8); 8.391 (3.8); 8.386 (2.6); 8.316 (0.4); 7.606 (3.2); 7.604
(3.2); 7.593 (3.2); 7.592 (3.2); 7.586 (3.1); 7.584 (3.1); 7.574 (3.0); 7.572 (2.9); 3.325 (129.9); 2.676 (0.7); 2.672 (1.0); 2.667
(0.7); 2.542 (9.8); 2.525 (3.1); 2.511 (61.2); 2.507 (117.8); 2.503 (150.9); 2.498 (109.1); 2.494 (53.1); 2.338 (0.4); 2.334 (0.8);
2.329 (1.0); 2.325 (0.8); 0.146 (0.5); 0.008 (5.5); 0.000 (120.8); −0.008 (5.0); −0.150 (0.6)
Compound No. I-3-16: ¹H NMR (400.0 MHz, d₆-DMSO):
11.889 (6.3); 9.202 (6.4); 9.197 (6.5); 8.800 (4.4); 8.796 (4.8); 8.788 (4.7); 8.784 (4.7); 8.635 (4.9); 8.632 (5.2); 8.624 (5.2); 8.620
(5.1); 8.475 (16.0); 8.418 (2.6); 8.413 (3.9); 8.408 (2.6); 8.398 (2.8); 8.393 (4.0); 8.388 (2.7); 8.351 (4.9); 8.347 (4.9); 8.331
(5.3); 8.327 (5.1); 8.317 (0.5); 7.735 (5.3); 7.724 (5.1); 7.715 (5.0); 7.703 (4.9); 7.605 (3.4); 7.593 (3.4); 7.586 (3.4); 7.573 (3.2);
3.326 (123.3); 2.676 (0.7); 2.672 (1.0); 2.667 (0.8); 2.542 (6.7); 2.525 (2.9); 2.511 (59.3); 2.507 (118.0); 2.503 (155.3); 2.498
(114.9); 2.334 (0.8); 2.329 (1.0); 2.325 (0.8); 0.146 (0.6); 0.008 (4.6); 0.000 (119.9); −0.008 (5.3); −0.150 (0.6)
Compound No. I-3-17: ¹H NMR (400.0 MHz, d₆-DMSO):
11.089 (2.3); 9.182 (1.9); 9.178 (1.9); 8.813 (1.5); 8.809 (1.6); 8.801 (1.6); 8.797 (1.6); 8.703 (3.0); 8.697 (3.5); 8.635 (3.4); 8.630
(2.9); 8.382 (0.8); 8.377 (1.1); 8.372 (0.8); 8.362 (0.8); 8.357 (1.2); 8.352 (0.8); 7.616 (1.1); 7.615 (1.1); 7.604 (1.1); 7.603
(1.1); 7.596 (1.1); 7.595 (1.1); 7.584 (1.0); 7.583 (1.0); 3.327 (19.1); 2.542 (1.5); 2.525 (0.6); 2.512 (11.2); 2.508 (22.0); 2.503
(28.5); 2.498 (20.6); 2.494 (9.9); 2.309 (16.0); 0.008 (1.0); 0.000 (24.8); −0.009 (0.9)
Compound No. I-3-18: ¹H NMR (400.0 MHz, d₆-DMSO):
11.099 (3.1); 9.185 (2.1); 8.818 (1.5); 8.806 (1.6); 8.383 (1.4); 8.363 (1.5); 8.256 (1.3); 8.236 (1.4); 8.183 (2.5); 7.846 (0.8); 7.826
(2.0); 7.807 (1.5); 7.783 (2.1); 7.764 (1.0); 7.625 (1.3); 7.613 (1.3); 7.605 (1.3); 7.593 (1.2); 3.340 (20.0); 2.672 (0.3); 2.507
(38.2); 2.503 (50.5); 2.499 (39.9); 2.333 (16.0); 2.210 (0.7); 0.000 (8.5)
Compound No. I-3-19: ¹H NMR (400.0 MHz, d₆-DMSO):
11.030 (2.7); 9.173 (2.1); 9.074 (0.5); 8.804 (1.7); 8.795 (1.9); 8.370 (1.2); 8.350 (1.3); 8.279 (0.4); 8.259 (0.4); 7.954 (2.7); 7.948
(2.8); 7.784 (0.4); 7.754 (2.3); 7.732 (3.6); 7.664 (2.2); 7.659 (2.1); 7.643 (1.4); 7.637 (1.4); 7.610 (1.3); 7.598 (1.4); 7.591
(1.3); 7.579 (1.3); 7.564 (0.4); 7.551 (0.5); 7.545 (0.5); 7.532 (0.4); 5.757 (0.5); 4.038 (0.5); 4.020 (0.5); 3.896 (0.5); 3.326 (34.5);
2.672 (0.3); 2.507 (40.7); 2.503 (51.6); 2.499 (39.2); 2.329 (0.4); 2.293 (16.0); 2.224 (0.3); 2.175 (1.3); 2.072 (0.4); 1.989 (2.1);
1.193 (0.6); 1.175 (1.1); 1.157 (0.6); 0.000 (9.2)
Compound No. I-3-20: ¹H NMR (400.0 MHz, d₆-DMSO):
11.438 (4.7); 9.171 (7.3); 9.167 (7.0); 8.843 (5.0); 8.839 (5.4); 8.831 (5.3); 8.827 (5.3); 8.379 (2.9); 8.374 (4.2); 8.369 (2.9); 8.359
(3.2); 8.353 (4.5); 8.349 (3.0); 8.317 (0.4); 8.086 (1.5); 8.078 (13.8); 8.073 (4.6); 8.061 (4.7); 8.056 (15.9); 8.048 (1.7); 7.734
(1.8); 7.727 (16.0); 7.722 (5.0); 7.710 (4.5); 7.704 (14.2); 7.697 (1.4); 7.639 (3.9); 7.627 (3.8); 7.619 (3.7); 7.607 (3.5); 3.329
(289.9); 2.996 (0.6); 2.712 (2.1); 2.677 (0.9); 2.672 (1.1); 2.667 (0.9); 2.583 (0.6); 2.543 (459.5); 2.525 (3.8); 2.512 (60.5); 2.507
(121.0); 2.503 (159.3); 2.498 (115.9); 2.494 (56.5); 2.368 (2.0); 2.334 (0.8); 2.330 (1.0); 2.325 (0.8); 0.146 (0.6); 0.008 (5.4);
0.000 (137.2); −0.009 (5.1); −0.150 (0.6)
Compound No. I-3-21: ¹H NMR (400.0 MHz, d₆-DMSO):
11.229 (16.0); 9.186 (11.6); 9.181 (11.6); 8.840 (9.0); 8.836 (9.4); 8.828 (9.5); 8.824 (9.2); 8.661 (10.2); 8.658 (10.6); 8.650 (10.7);
8.646 (10.5); 8.393 (11.5); 8.390 (15.0); 8.385 (7.5); 8.380 (5.1); 8.373 (12.9); 8.369 (15.5); 8.365 (7.8); 8.360 (4.9); 7.790
(11.5); 7.778 (10.9); 7.770 (10.6); 7.758 (10.5); 7.639 (6.6); 7.638 (6.4); 7.627 (6.5); 7.626 (6.4); 7.619 (6.4); 7.607 (6.1); 7.606
(5.9); 3.333 (133.0); 2.678 (0.4); 2.674 (0.6); 2.669 (0.4); 2.544 (13.4); 2.527 (1.6); 2.514 (33.1); 2.509 (65.7); 2.505 (85.4);
2.500 (60.9); 2.496 (28.7); 2.336 (0.4); 2.332 (0.6); 2.327 (0.4); 0.146 (0.4); 0.008 (3.0); 0.000 (80.0); −0.009 (2.7); −0.150
(0.4)
Compound No. I-3-22: ¹H NMR (400.0 MHz, d₆-DMSO):
11.050 (2.3); 9.177 (1.8); 9.173 (1.8); 8.810 (1.4); 8.806 (1.5); 8.798 (1.5); 8.794 (1.5); 8.378 (0.7); 8.373 (1.1); 8.369 (0.7); 8.358
(0.8); 8.353 (1.1); 8.349 (0.7); 7.858 (2.5); 7.852 (2.7); 7.792 (2.6); 7.771 (3.7); 7.676 (2.2); 7.670 (2.0); 7.654 (1.5); 7.648
(1.4); 7.613 (1.2); 7.601 (1.2); 7.593 (1.1); 7.581 (1.1); 3.328 (32.3); 2.526 (0.5); 2.512 (10.1); 2.508 (19.8); 2.503 (25.5); 2.499
(18.3); 2.495 (8.8); 2.302 (16.0); 1.990 (0.6); 1.259 (0.4); 1.234 (0.5); 1.175 (0.3); 0.000 (2.7)
Compound No. I-3-23: ¹H NMR (400.0 MHz, d₆-DMSO):
11.004 (2.7); 9.176 (2.3); 9.070 (0.4); 8.804 (1.8); 8.795 (2.0); 8.792 (1.9); 8.375 (1.3); 8.355 (1.4); 7.750 (1.3); 7.743 (1.2); 7.735
(1.0); 7.731 (1.3); 7.727 (1.9); 7.718 (0.5); 7.708 (1.4); 7.703 (1.1); 7.699 (1.1); 7.692 (1.4); 7.685 (1.8); 7.610 (1.4); 7.598
(2.0); 7.590 (1.7); 7.585 (2.3); 7.579 (4.5); 7.570 (3.5); 7.560 (3.0); 7.556 (1.8); 7.542 (0.8); 7.531 (0.4); 3.327 (19.0); 2.503 (32.1);
2.293 (16.0); 2.224 (0.5); 2.072 (0.5); 0.000 (5.5)
Compound No. I-3-24: ¹H NMR (400.0 MHz, d₆-DMSO):
11.847 (6.7); 9.191 (4.7); 9.187 (4.8); 8.798 (3.1); 8.794 (3.4); 8.786 (3.4); 8.782 (3.3); 8.500 (16.0); 8.408 (2.5); 8.403 (3.5); 8.398
(2.6); 8.388 (2.7); 8.382 (3.8); 8.378 (2.7); 8.317 (0.4); 7.779 (0.9); 7.763 (1.9); 7.757 (1.6); 7.747 (1.2); 7.742 (3.7); 7.736
(1.3); 7.726 (1.7); 7.720 (2.3); 7.704 (1.1); 7.601 (3.1); 7.589 (3.1); 7.583 (3.0); 7.581 (3.1); 7.571 (2.9); 7.569 (3.0); 7.501 (3.0);
7.497 (5.6); 7.476 (9.5); 7.455 (4.5); 7.452 (2.8); 3.326 (100.2); 2.676 (0.6); 2.672 (0.8); 2.667 (0.6); 2.542 (10.4); 2.525 (2.1);
2.512 (46.5); 2.507 (94.5); 2.503 (125.0); 2.498 (90.7); 2.494 (44.1); 2.334 (0.6); 2.330 (0.8); 2.325 (0.6); 0.146 (0.5); 0.008
(4.0); 0.000 (110.6); −0.009 (4.0); −0.150 (0.5)
Compound No. I-4-2: ¹H NMR (400.0 MHz, d₆-DMSO):
11.606 (2.0); 9.140 (2.1); 9.135 (2.1); 8.805 (1.4); 8.801 (1.5); 8.793 (1.6); 8.789 (1.5); 8.352 (0.9); 8.348 (1.3); 8.343 (0.9); 8.333
(1.0); 8.327 (1.3); 8.323 (0.9); 7.602 (1.1); 7.601 (1.1); 7.590 (1.1); 7.589 (1.1); 7.583 (1.1); 7.570 (1.0); 7.569 (1.0); 4.368
(16.0); 3.326 (4.5); 2.508 (11.2); 2.503 (14.2); 2.499 (10.5); 0.000 (3.2)
Compound No. I-4-3: ¹H NMR (400.0 MHz, d₆-DMSO):
11.696 (4.8); 9.120 (4.3); 9.115 (4.4); 8.796 (3.1); 8.792 (3.2); 8.784 (3.3); 8.780 (3.1); 8.563 (4.2); 8.557 (4.3); 8.332 (1.8); 8.328
(2.6); 8.322 (1.7); 8.312 (1.9); 8.308 (2.7); 8.302 (1.8); 7.936 (2.6); 7.930 (2.5); 7.915 (2.9); 7.909 (2.9); 7.616 (5.1); 7.595
(4.8); 7.580 (2.4); 7.572 (2.4); 7.560 (2.3); 6.030 (16.0); 3.330 (58.9); 2.673 (0.3); 2.526 (0.8); 2.508 (41.4); 2.504 (53.0); 2.499
(38.1); 2.331 (0.3); 0.008 (0.7); 0.000 (17.5); −0.008 (0.7)
Compound No. I-4-4: ¹H NMR (400.0 MHz, d₆-DMSO):
12.471 (0.9); 9.118 (0.8); 8.848 (0.6); 8.837 (0.6); 8.326 (0.5); 8.321 (0.7); 8.306 (0.5); 8.301 (0.7); 8.296 (0.4); 7.630 (0.6); 7.618
(0.6); 7.611 (0.6); 7.598 (0.6); 7.220 (0.7); 7.093 (0.7); 6.965 (0.7); 6.454 (3.1); 3.814 (16.0); 3.332 (20.4); 2.507 (33.8); 2.502
(43.8); 2.498 (32.1); 1.260 (0.8); 1.245 (0.5); 0.008 (1.3); 0.000 (32.0); −0.008 (1.6)
Compound No. I-4-5: ¹H NMR (400.0 MHz, d₆-DMSO):
11.744 (3.7); 9.148 (3.1); 9.147 (3.2); 9.143 (3.3); 8.809 (2.5); 8.805 (2.6); 8.797 (2.6); 8.793 (2.5); 8.361 (1.4); 8.356 (1.9); 8.351
(1.4); 8.341 (1.5); 8.335 (2.0); 8.331 (1.5); 7.606 (1.9); 7.605 (1.8); 7.594 (1.8); 7.593 (1.8); 7.587 (1.8); 7.585 (1.7); 7.574
(1.7); 7.573 (1.7); 5.829 (14.9); 4.251 (2.2); 4.233 (7.0); 4.215 (7.1); 4.198 (2.3); 3.329 (26.3); 2.525 (0.6); 2.512 (10.7); 2.507
(21.5); 2.503 (28.2); 2.498 (20.3); 2.494 (9.6); 2.076 (0.4); 1.258 (7.9); 1.240 (16.0); 1.222 (7.6); 0.008 (0.4); 0.000 (13.3); −0.009
(0.4)
Compound No. I-4-6: ¹H NMR (400.0 MHz, d₆-DMSO):
11.716 (5.7); 9.156 (4.0); 9.151 (4.1); 9.030 (2.2); 9.011 (2.3); 8.880 (2.7); 8.868 (2.7); 8.821 (2.7); 8.818 (2.9); 8.809 (2.9); 8.806
(2.9); 8.480 (0.6); 8.460 (1.3); 8.441 (0.7); 8.397 (1.5); 8.392 (2.2); 8.388 (1.6); 8.377 (1.7); 8.372 (2.4); 8.368 (1.6); 7.973
(1.7); 7.957 (2.1); 7.954 (2.1); 7.938 (1.5); 7.639 (2.0); 7.627 (2.1); 7.619 (2.0); 7.607 (2.0); 7.361 (16.0); 7.354 (8.6); 7.347 (8.2);
7.331 (1.8); 7.286 (0.5); 7.274 (1.2); 7.267 (1.6); 7.260 (1.7); 7.253 (1.9); 7.245 (1.4); 7.239 (1.0); 7.232 (0.6); 5.495 (12.6); 5.146
(0.4); 4.973 (0.4); 4.955 (1.6); 4.937 (2.4); 4.918 (1.7); 4.901 (0.5); 4.137 (0.3); 4.130 (0.3); 4.085 (0.4); 4.062 (0.4); 4.027
(0.4); 4.007 (0.4); 3.988 (0.4); 3.918 (0.4); 3.908 (0.4); 3.862 (0.3); 3.843 (0.3); 3.837 (0.3); 3.820 (0.3); 3.793 (0.4); 2.507 (35.7);
2.503 (45.5); 2.499 (36.0); 2.330 (0.3); 1.425 (9.9); 1.407 (10.0); 1.379 (0.6); 0.000 (28.5)
Compound No. I-4-7: ¹H NMR (400.0 MHz, d₆-DMSO):
11.918 (3.7); 9.129 (5.7); 9.124 (5.9); 8.934 (1.7); 8.920 (3.4); 8.907 (1.8); 8.848 (3.7); 8.844 (3.9); 8.836 (4.0); 8.832 (3.9); 8.310
(2.2); 8.306 (3.3); 8.301 (2.3); 8.290 (2.4); 8.286 (3.5); 8.281 (2.4); 7.977 (3.7); 7.957 (4.1); 7.934 (3.8); 7.915 (4.1); 7.839
(3.8); 7.819 (4.2); 7.619 (2.9); 7.607 (3.0); 7.599 (3.0); 7.587 (2.7); 7.521 (1.7); 7.504 (3.9); 7.487 (3.2); 7.484 (3.2); 7.479 (3.2);
7.475 (3.2); 7.458 (3.5); 7.441 (1.5); 7.391 (2.8); 7.375 (5.2); 7.343 (4.3); 7.324 (4.7); 7.306 (2.3); 5.281 (16.0); 4.746 (7.8); 4.733
(7.9); 3.326 (17.4); 2.671 (0.6); 2.506 (70.6); 2.502 (90.7); 2.498 (70.6); 2.329 (0.6); 2.074 (0.5); 0.146 (0.4); 0.000 (73.7);
−0.150 (0.4)
Compound No. I-4-8: ¹H NMR(601.6 MHz, d₆-DMSO):
11.671 (2.9); 9.140 (5.9); 9.136 (6.2); 8.804 (4.0); 8.801 (4.8); 8.796 (4.2); 8.793 (4.8); 8.349 (2.0); 8.345 (3.5); 8.342 (2.6); 8.336
(2.3); 8.332 (3.7); 8.329 (2.7); 7.599 (3.5); 7.591 (3.5); 7.586 (3.5); 7.578 (3.4); 6.148 (0.9); 6.137 (1.6); 6.131 (1.1); 6.126
(0.6); 6.121 (2.7); 6.110 (2.1); 6.103 (1.0); 6.100 (1.2); 6.093 (2.0); 6.082 (1.1); 5.384 (4.5); 5.383 (4.3); 5.367 (3.6); 5.366 (4.5);
5.356 (16.0); 5.350 (5.2); 5.347 (8.5); 5.345 (7.1); 5.327 (3.4); 5.325 (3.6); 3.356 (7.6); 3.352 (15.0); 3.347 (18.9); 3.344 (20.3);
3.343 (20.3); 2.523 (0.6); 2.507 (34.7); 2.504 (49.0); 2.502 (36.9); 0.005 (1.4); 0.000 (43.7); −0.006 (1.8)
Compound No. I-4-9: ¹H NMR (400.0 MHz, d₆-DMSO):
11.621 (4.0); 9.142 (3.8); 9.136 (3.9); 8.805 (2.7); 8.801 (2.8); 8.793 (3.0); 8.789 (2.7); 8.353 (1.6); 8.349 (2.3); 8.343 (1.5); 8.333
(1.7); 8.329 (2.4); 8.323 (1.5); 7.603 (2.3); 7.591 (2.2); 7.583 (2.2); 7.571 (2.1); 5.758 (1.1); 4.654 (5.1); 4.637 (10.4); 4.620
(5.0); 3.333 (30.8); 3.082 (0.7); 2.966 (0.6); 2.527 (0.4); 2.513 (9.7); 2.509 (19.0); 2.504 (24.5); 2.500 (17.3); 2.002 (0.6); 1.990
(1.5); 1.985 (2.6); 1.967 (5.3); 1.949 (5.3); 1.931 (2.8); 1.913 (0.6); 1.193 (0.3); 1.176 (0.7); 1.158 (0.3); 0.909 (7.9); 0.890 (16.0);
0.872 (7.4); 0.861 (0.4); 0.840 (0.4); 0.594 (0.5); 0.008 (0.8); 0.000 (20.3); −0.009 (0.7)
Compound No. I-4-10: ¹H NMR (400.0 MHz, d₆-DMSO):
11.701 (1.6); 9.042 (4.5); 9.037 (4.6); 8.831 (3.1); 8.827 (3.3); 8.819 (3.3); 8.815 (3.2); 8.270 (1.7); 8.265 (2.6); 8.260 (1.8); 8.250
(1.9); 8.245 (2.8); 8.240 (1.8); 7.621 (2.5); 7.609 (2.5); 7.601 (2.4); 7.589 (2.3); 7.371 (0.5); 7.366 (0.4); 7.359 (1.2); 7.348
(4.7); 7.343 (4.6); 7.335 (9.7); 7.331 (10.1); 7.254 (5.1); 7.247 (4.2); 7.235 (3.8); 7.231 (3.0); 5.613 (16.0); 3.333 (22.2); 2.672
(0.4); 2.507 (48.6); 2.502 (62.4); 2.498 (45.6); 2.329 (0.4); 0.000 (6.7)
Compound No. I-4-11: ¹H NMR (400.0 MHz, d₆-DMSO):
11.681 (6.2); 9.142 (4.7); 9.138 (4.7); 8.806 (3.2); 8.802 (3.4); 8.794 (3.4); 8.790 (3.2); 8.464 (1.3); 8.452 (2.3); 8.439 (1.3); 8.358
(2.0); 8.353 (2.8); 8.348 (2.0); 8.338 (2.2); 8.333 (2.9); 8.328 (1.9); 7.605 (2.5); 7.593 (2.5); 7.585 (2.5); 7.573 (2.3); 5.410
(16.0); 3.352 (12.7); 3.149 (2.2); 3.132 (5.1); 3.117 (5.1); 3.100 (2.3); 2.507 (27.0); 2.503 (34.1); 2.499 (25.8); 1.470 (0.8); 1.454
(2.6); 1.446 (1.5); 1.436 (4.0); 1.417 (3.6); 1.399 (1.5); 1.361 (0.8); 1.344 (2.7); 1.325 (3.7); 1.305 (3.6); 1.288 (2.0); 1.270 (0.6);
0.906 (8.2); 0.888 (15.8); 0.870 (6.8); 0.008 (3.4); 0.000 (38.9)

²) The NMR data for selected examples are listed either in conventional form (δ values, number of hydrogen atoms, multiplet splitting) or as NMR peak lists.

### NMR Peak List Method

When the 1H NMR data for selected examples are noted in the form of 1H NMR peak lists, first the δ value in ppm and then the signal intensity in round brackets are listed for each signal peak. The δ value−signal intensity number pairs for different signal peaks are listed with separation from one another by semicolons. The peak list for one example therefore takes the form of:

δ₁(intensity₁);δ₂(intensity₂); . . . ;δᵢ(intensityᵢ); . . . ;δₙ(intensityₙ)

The intensity of sharp signals correlates with the height of the signals in a printed example of an NMR spectrum in cm and shows the true ratios of the signal intensities. In the case of broad signals, several peaks or the middle of the signal and their relative intensities may be shown in comparison to the most intense signal in the spectrum. For calibration of the chemical shift of the 1H NMR spectra we use tetramethylsilane and/or the chemical shift of the solvent, particularly in the case of spectra measured in DMSO. Therefore, the tetramethylsilane peak may but need not occur in the NMR peak lists. The lists of the 1H NMR peaks are similar to the conventional 1H NMR printouts and thus usually contain all peaks listed in a conventional NMR interpretation. In addition, like conventional 1H NMR printouts, they may show solvent signals, signals of stereoisomers of the target compounds, which likewise form part of the subject-matter of the invention, and/or peaks of impurities. In the reporting of compound signals in the delta range of solvents and/or water, our lists of 1H NMR peaks show the usual solvent peaks, for example peaks of DMSO in DMSO-d₆ and the peak of water, which usually have a high intensity on average. The peaks of stereoisomers of the target compounds and/or peaks of impurities usually have a lower intensity on average than the peaks of the target compounds (for example with a purity of >90%). Such stereoisomers and/or impurities may be typical of the particular preparation process. Their peaks can thus help in this case to identify reproduction of our preparation process with reference to “by-product fingerprints”. An expert calculating the peaks of the target compounds by known methods (MestreC, ACD simulation, but also with empirically evaluated expected values) can, if required, isolate the peaks of the target compounds, optionally using additional intensity filters. This isolation would be similar to the relevant peak picking in conventional 1H NMR interpretation. Further details of 1H NMR peak lists can be found in Research Disclosure Database Number 564025.

The NMR spectra were determined using a Bruker Avance 400 fitted with a flow probe head (volume 60 μl). Solvents used were CD₃CN, CDCl₃ DMF-D₇ or DMSO-D₆, and tetramethylsilane (0.00 ppm) was used as reference. In individual cases, the NMR spectra were determined using a Bruker Avance II 600. In the case of NMR data in the conventional form, the splitting of the signals was described as follows: s (singlet), d (doublet), t (triplet), q (quartet), m (multiplet).

### BIOLOGICAL EXAMPLES

The examples which follow show the biological action of the inventive compounds. In these examples, the inventive compounds cited relate to the compounds listed in Tables 1 to 4 with the corresponding reference numerals, e.g. I-1-35:

Myzus persicae—spray test

Solvent: 78 parts by weight of acetone
1.5 parts by weight of dimethylformamide

Emulsifier: 0.5 part by weight of alkylaryl polyglycol ether

To produce an appropriate active ingredient formulation, 1 part by weight of active ingredient is mixed with the specified amounts of solvent and emulsifier, and the concentrate is diluted to the desired concentration with emulsifier-containing water.

Discs of Chinese cabbage leaves (Brassica pekinensis) infested by all stages of the green peach aphid (Myzus persicae) are sprayed with an active ingredient formulation of the desired concentration.

After 6 days, the efficacy in % is determined. 100% means that all the aphids have been killed; 0% means that no aphids have been killed.

In this test, for example, the following compounds from the preparation examples show an efficacy of 100% at an application rate of 500 g/ha: I-1-2, I-1-4, I-1-9, I-1-18, I-1-19, I-1-22, I-1-23, I-1-25, I-1- 33, I-1-34, I-1-38, I-1-44, I-1-46, I-1-60, I-1-63, I-1-147, I-1-153, I-1-163, I-1-166, I-1-169, I-1-173, I-1-174, I-1-175, I-1-176, I-1-178, I-1-179, I-1-180, I-1-182, I-1-185, I-1-186, I-1-190, I-1-192, I- 1-205, I-1-214, I-1-218, I-1-223, I-1-224, I-1-225, I-1-226, I-1-242, I-1-243, I-1-245, I-1-246, I-1-261, I- 1-268, I-1-277, I-1-283, I-1-286, I-1-292, I-1-295, I-1-304, I-1-310, I-1-321, I-1-344, I-1-346, I-2-7 In this test, for example, the following compounds from the preparation examples show an efficacy of 90% at an application rate of 500 g/ha: I-1-1, I-1-3, I-1-5, I-1-6, I-1-7, I-1-8, I-1-10, I-1-13, I-1-15, I-1-16, I-1-17, I-1-20, I-1-26, I-1-27, I-1-28, I-1-30, I-1-31, I-1-32, I-1-36, I-1-39, I-1-40, I-1-41, I-1-42, I-1-43, I-1-46, I-1-50, I-1-51, I-1-52, I-1-54, I-1-64, I-1-65, I-1-123, I-1-124, I-1-126, I-1- 128, I-1-129, I-1-130, I-1-131, I-1-133, I-1-140, I-1-143, I-1-144, I-1-148, I-1-149, I-1-154, I-1-155, I-1-156, I- 1-164, I-1-167, I-1-170, I-1-172, I-1-181, I-1-183, I-1-184, I-1-187, I-1-188, I-1-189, I-1-191, I-1-196, I- 1-198, I-1-202, I-1-203, I-1-204, I-1-211, I-1-212, I-1-215, I-1-216, I-1-220, I-1-221, I-1-227, I-1-228, I- 1-229, I-1-231, I-1-233, I-1-234, I-1-237, I-1-239, I-1-244, I-1-259, I-1-260, I-1-262, I-1-264, I-1-266, I- 1-279, I-1-281, I-1-282, I-1-285, I-1-287, I-1-289, I-1-291, I-1-293, I-1-296, I-1-297, I-1-298, I-1-308, I- 1-311, I-1-312, I-1-313, I-1-314, I-1-317, I-1-318, I-1-320, I-1-329, I-1-330, I-1-333, I-1-335, I-1-338, I- 1-342, I-1-345, I-1-352, I-2-4, I-2-16

In this test, for example, the following compounds from the preparation examples show an efficacy of 80% at an application rate of 500 g/ha: I-1-11, I-1-12, I-1-14, I-1-49, I-1-55, I-1-61

Phaedon cochleariae—Spray Test (PHAECO)

Solvent: 78.0 parts by weight of acetone
1.5 parts by weight of dimethylformamide

Emulsifier: alkylaryl polyglycol ether

To produce an appropriate active ingredient formulation, 1 part by weight of active ingredient is dissolved using the specified parts by weight of solvent and made up with water containing an emulsifier concentration of 1000 ppm until the desired concentration is attained. To produce further test concentrations, the preparation is diluted with emulsifier-containing water.

Discs of Chinese cabbage leaves (Brassica pekinensis) are sprayed with an active ingredient formulation of the desired concentration and, after drying, populated with larvae of the mustard beetle (Phaedon cochleariae).

After 7 days, the efficacy in % is determined. 100% means that all the beetle larvae have been killed; 0% means that no beetle larvae have been killed.

In this test, for example, the following compounds from the preparation examples showed an efficacy of 100% at an application rate of 500 g/ha: I-1-48, I-1-130, I-1-131, I-1-346

In this test, for example, the following compounds from the preparation examples showed an efficacy of 83% at an application rate of 500 g/ha: I-1-22

Spodoptera frugiperda—Spray Test (SPODFR)

Solvent: 78.0 parts by weight of acetone
1.5 parts by weight of dimethylformamide

Emulsifier: alkylaryl polyglycol ether

To produce an appropriate active ingredient formulation, 1 part by weight of active ingredient is dissolved using the specified parts by weight of solvent and made up with water containing an emulsifier concentration of 1000 ppm until the desired concentration is attained. To produce further test concentrations, the preparation is diluted with emulsifier-containing water.

Leaf discs of maize (Zea mays) are sprayed with an active ingredient formulation of the desired concentration and, after drying, populated with caterpillars of the armyworm (Spodopterafrugiperda).

After 7 days, the efficacy in % is determined. 100% means that all the caterpillars have been killed; 0% means that no caterpillars have been killed.

In this test, for example, the following compounds from the preparation examples showed an efficacy of 100% at an application rate of 500 g/ha: I-1-48, I-1-130, I-1-131

In this test, for example, the following compounds from the preparation examples showed an efficacy of 83% at an application rate of 500 g/ha: I-1-22

Tetranychus urticae—spray test, OP-resistant (TETRUR)

Solvent: 78.0 parts by weight of acetone
1.5 parts by weight of dimethylformamide

Emulsifier: alkylaryl polyglycol ether

To produce an appropriate active ingredient formulation, 1 part by weight of active ingredient is dissolved using the specified parts by weight of solvent and made up with water containing an emulsifier concentration of 1000 ppm until the desired concentration is attained. To produce further test concentrations, the preparation is diluted with emulsifier-containing water.

Discs of bean leaves (Phaseolus vulgaris) infested by all stages of the greenhouse red spider mite (Tetranychus urticae) are sprayed with an active ingredient formulation of the desired concentration.

After 6 days, the efficacy in % is determined. 100% means that all the spider mites have been killed; 0% means that no spider mites have been killed.

In this test, for example, the following compounds from the preparation examples showed, at an application rate of 500 g/ha, an efficacy of 90%: I-1-131, I-1-205, I-1-210, I-1-282

Myzus persicae—Sachet Test

Solvent: 2% by vol. of N-methylpyrrolidone
2% by vol. of diacetone alcohol

To produce an appropriate active ingredient formulation, the active ingredient is mixed with the specified amounts of solvent and the concentrate is diluted to the desired concentration with water.

The active ingredient formulation is added to a specific nutrient medium in the desired concentration by pipetting. This plate is sealed and combined with a further plate populated with the green peach aphid (Myzus persicae).

After the desired time, the kill in % is determined. 100% means that all the animals have been killed; 0% means that no animals have been killed.

In this test, for example, the following compounds from the preparation examples showed an efficacy of 100% at an application rate of 30 ppm: I-1-1, I-1-2, I-1-3, I-1-4, I-1-5, I-1-6, I-1-7, I-1-8, I-1-9, I-1-10, I-1-11, I-1-12, I-1-13, I-1-14, I-1-15, I-1-16, I-1-17, I-1-18, I-1-19, I-1-20, I-1-21, I-1-22, I-1-23, I-1-24, I-1-25, I-1-26, I-1-27, I-28, I-1-29, I-1-30, I-1-31, I-1-32, I-1-33, I-1-34, I-1-35, I-1-36, I-1-37, I-1-38, I-1-39, I-1-40, I-1-42, I-1-43, I-1-44, I-1-45, I-1-46, I-1-48, I-1-49, I-1-50, I-1-51, I-1-52, I-1-53, I-1-54, I-1-55, I-1-56, I-1-57, I-1-58, I-1-60, I-1-61, I-1-62, I-1-63, I-1-64, I-1-65, I-1-66, I-1-67, I-1-68, I-1-69, I-1-70, I-1-71, I-1-72, I-1-73, I-1-74, I-1-75, I-1-76, I-1-77, I-1-78, I-1-79, I-1-80, I-1-81, I-1-82, I-1-83, I-1-84, I-1-85, I-1-86, I-1-87, I-1-88, I-1-89, I-1-90, I-1-91, I-1-92, I-1-93, I-1-95, I-1-99, I-1-100, I-1-108, I-1-111, I-1-116, I-1-119, I-1-120, I-1-122, I-1-123, I-1-124, I-1-125, I-1-126, I-1-128, I-1- 129, I-1-130, I-1-131, I-1-132, I-1-133, I-1-135, I-1-136, I-1-138, I-1-139, I-1-140, I-1-142, I-1-143, I-1- 144, I-1-145, I-1-146, I-1-147, I-1-148, I-1-149, I-1-153, I-1-154, I-1-155, I-1-156, I-1-157, I-1-158, I-1- 159, I-1-160, I-1-161, I-1-162, I-1-163, I-1-164, I-1-165, I-1-166, I-1-167, I-1-168, I-1-169, I-1-170, I-1- 171, I-1-172, I-1-174, I-1-175, I-1-177, I-1-180, I-1-182, I-1-183, I-1-184, I-1-185, I-1-186, I-1-187, I-1- 188, I-1-189, I-1-190, I-1-191, I-1-192, I-1-194, I-1-195, I-1-196, I-1-197, I-1-198, I-1-199, I-1-202, I-1- 203, I-1-205, I-1-207, I-1-208, I-1-209, I-1-211, I-1-212, I-1-213, I-1-214, I-1-215, I-1-216, I-1-217, I-1- 218, I-1-219, I-1-220, I-1-221, I-1-223, I-1-224, I-1-225, I-1-226, I-1-227, I-1-228, I-1-229, I-1-231, I-1- 232, I-1-233, I-1-234, I-1-235, I-1-236, I-1-237, I-1-238, I-1-239, I-1-241, I-1-242, I-1-243, I-1-244, I-1- 245, I-1-246, I-1-247, I-1-249, I-1-257, I-1-259, I-1-260, I-1-261, I-1-262, I-1-263, I-1-264, I-1-265, I-1- 266, I-1-267, I-1-268, I-1-269, I-1-274, I-1-276, I-1-277, I-1-278, I-1-279, I-1-280, I-1-281, I-1-282, I-1- 283, I-1-284, I-1-285, I-1-286, I-1-287, I-1-288, I-1-289, I-1-290, I-1-291, I-1-292, I-1-293, I-1-294, I-1- 295, I-1-296, I-1-297, I-1-298, I-1-299, I-1-300, I-1-301, I-1-302, I-1-304, I-1-305, I-1-306, I-1-307, I-1- 308, I-1-309, I-1-310, I-1-311, I-1-312, I-1-313, I-1-314, I-1-315, I-1-316, I-1-317, I-1-318, I-1-319, I-1- 320, I-1-321, I-1-322, I-1-323, I-1-324, I-1-325, I-1-326, I-1-327, I-1-328, I-1-329, I-1-330, I-1-331, I-1- 332, I-1-333, I-1-334, I-1-335, I-1-336, I-1-338, I-1-339, I-1-340, I-1-341, I-1-342, I-1-344, I-1-345, I-1- 346, I-1-347, I-1-349, I-1-351, I-1-352, I-1-353, I-2-1, I-2-4, I-2-6, I-2-14, I-2-16, I-2-17, I-2-19, I-2-23, I-2-26, I-3-1, I-3-2, I-3-5, I-3-6, I-3-7, I-3-9, I-3-10, I-3-14, I-3-16, I-4-1, I-4-2, I-4-3, I-4-6, I-4-8

In this test, for example, the following compounds from the preparation examples showed an efficacy of 80% at an application rate of 30 ppm: I-1-104, I-1-173, I-1-176, I-1-178, I-1-181, I-1-204, I-1-210, I-1-270, I-1-348, I-1-350, I-2-18, I-2-22

In this test, for example, the following compounds from the preparation examples showed an efficacy of 100% at an application rate of 6 ppm: I-1-1, I-1-2, I-1-3, I-1-4, I-1-5, I-1-6, I-1-7, I-1-8, I-1-9, I-1-10, I-1-11, I-1-12, I-1-13, I-1-14, I-1-15, I-1-17, I-1-18, I-1-19, I-1-20, I-1-21, I-1-22, I-1-23, I-1-24, I-1-25, I-1-26, I-1-27, I-1-28, I-1-29, I-1-30, I-1-31, I-1-32, I-1-33, I-1-34, I-1-36, I-1-37, I-1-38, I-1-39, I-1-40, I-1-43, I-1-44, I-1-45, I-1-46, I-1-49, I-1-51, I-1-52, I-1-53, I-1-54, I-1-57, I-1-60, I-1-62, I-1-64, I-1-65, I-1-67, I-1-72, I-1-74, I-1-76, I-1-77, I-1-84, I-1-85, I-1-88, I-1-89, I-1-90, I-1-111, I-1-116, I-1-120, I-1-122, I-1-123, I-1-124, I-1-126, I-1-128, I-1-129, I-1-130, I-1-131, I-1-133, I-1-139, I-1-140, I- 1-142, I-1-143, I-1-144, I-1-146, I-1-147, I-1-148, I-1-149, I-1-153, I-1-154, I-1-155, I-1-156, I-1-159, I- 1-160, I-1-162, I-1-163, I-1-164, I-1-165, I-1-166, I-1-167, I-1-168, I-1-169, I-1-170, I-1-172, I-1-174, I- 1-175, I-1-182, I-1-183, I-1-184, I-1-185, I-1-186, I-1-187, I-1-188, I-1-189, I-1-190, I-1-191, I-1-192, I- 1-195, I-1-196, I-1-197, I-1-198, I-1-207, I-1-211, I-1-212, I-1-214, I-1-215, I-1-216, I-1-217, I-1-218, I- 1-219, I-1-220, I-1-221, I-1-223, I-1-224, I-1-225, I-1-226, I-1-227, I-1-228, I-1-229, I-1-231, I-1-233, I- 1-234, I-1-236, I-1-237, I-1-238, I-1-239, I-1-241, I-1-242, I-1-243, I-1-244, I-1-245, I-1-246, I-1-247, I- 1-259, I-1-260, I-1-261, I-1-262, I-1-263, I-1-264, I-1-265, I-1-266, I-1-268, I-1-269, I-1-276, I-1-277, I- 1-279, I-1-282, I-1-283, I-1-284, I-1-285, I-1-286, I-1-287, I-1-288, I-1-289, I-1-290, I-1-291, I-1-292, I- 1-293, I-1-295, I-1-296, I-1-298, I-1-299, I-1-300, I-1-302, I-1-304, I-1-307, I-1-308, I-1-309, I-1-310, I- 1-312, I-1-313, I-1-314, I-1-315, I-1-316, I-1-317, I-1-318, I-1-319, I-1-320, I-1-321, I-1-322, I-1-323, I- 1-324, I-1-325, I-1-326, I-1-327, I-1-328, I-1-329, I-1-330, I-1-331, I-1-332, I-1-333, I-1-334, I-1-335, I- 1-336, I-1-338, I-1-340, I-1-341, I-1-342, I-1-344, I-1-345, I-1-346, I-1-353, I-3-1, I-3-14, I-4-3, I-4-6, I-4-8

In this test, for example, the following compounds from the preparation examples showed an efficacy of 80% at an application rate of 6 ppm: I-1-42, I-1-58, I-1-80, I-1-104, I-1-161, I-1-171, I-1-173, I-1-176, I-1-180, I-1-202, I-1-203, I-1-204, I-1-209, I-1-235, I-1-280, I-1-281, I-1-347, I-3-7

1. A compound of formula (I) 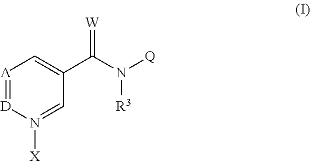
in which
A is N or CR¹,
D is N or CR², where A and D are not both N,
R¹ is a radical from the group of hydrogen, halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl,
R² is a radical from the group of hydrogen, halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl,
X is a free electron pair or oxygen,
W is oxygen, sulphur or NR⁴,
R³ is a radical from the group of hydrogen, cyano, hydroxyl, C₁-C₆-alkyl, C₃-C₆-cycloalkyl, C₁-C₆-haloalkyl, C₃-C₆-halocycloalkyl, C₂-C₆-alkenyl, C₂-C₆-haloalkenyl, C₃-C₆-alkynyl, C₃-C₆-haloalkynyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, C₁-C₆-alkylcarbonyl, C₂-C₆-alkenylcarbonyl, C₃-C₆-alkynylcarbonyl, C₁-C₆-haloalkylcarbonyl, C₃-C₆-cycloalkylcarbonyl, C₃-C₆-cycloalkyl-C₁-C₆-alkylcarbonyl, C₂-C₆-haloalkenylcarbonyl, C₃-C₆-halocycloalkylcarbonyl, C₁-C₆-alkoxy-C₁-C₆-alkylcarbonyl, C₁-C₆-alkoxycarbonyl, C₁-C₆-haloalkoxycarbonyl, C₁-C₆-alkylsulphonyl, C₁-C₆-haloalkylsulphonyl, C₁-C₆-alkylaminocarbonyl, di(C₁-C₆)alkylaminocarbonyl, C₁-C₆-alkylcarbonyloxy, C₁-C₆-alkoxycarbonyloxy, aryl, hetaryl, arylcarbonyl, hetarylcarbonyl, aryl-C₁-C₆-alkyl, hetaryl-C₁-C₆-alkyl, aryl-C₁-C₆-alkylcarbonyl, hetaryl-C₁-C₆-alkylcarbonyl, where the aryl, hetaryl, aryl-C₁-C₆-alkyl, hetaryl-C₁-C₆-alkyl, arylcarbonyl, hetarylcarbonyl, aryl-C₁-C₆-alkylcarbonyl, hetaryl-C₁-C₆-alkylcarbonyl substituents are optionally mono- or polysubstituted, identically or differently, by halogen, cyano, nitro, hydroxyl, C₁-C₆-alkyl, C₂-C₆-alkenyl, C₃-C₆-alkynyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl,
R⁴ is a radical from the group of hydrogen, cyano, nitro, hydroxyl, C₁-C₆-alkyl, C₃-C₆-cycloalkyl, C₁-C₆-haloalkyl, C₃-C₆-halocycloalkyl, C₂-C₆-alkenyl, C₂-C₆-haloalkenyl, C₃-C₆-alkynyl, C₃-C₆-haloalkynyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, C₁-C₆-alkylcarbonyl, C₁-C₆-haloalkylcarbonyl, C₁-C₆-alkoxycarbonyl, C₁-C₆-alkylcarbonyloxy, C₁-C₆-alkylsulphonyl, C₁-C₆-haloalkylsulphonyl, aryl, hetaryl, aryl-C₁-C₆-alkyl, hetaryl-C₁-C₆-alkyl, arylcarbonyl, hetarylcarbonyl, where the aryl, hetaryl, aryl-C₁-C₆-alkyl, hetaryl-C₁-C₆-alkyl, arylcarbonyl, hetarylcarbonyl substituents are optionally mono- or polysubstituted, identically or differently, by halogen, cyano, nitro, hydroxyl, C₁-C₆-alkyl, C₂-C₆-alkenyl, C₃-C₆-alkynyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl,
Q is one of the Q-1 to Q-4 radicals: 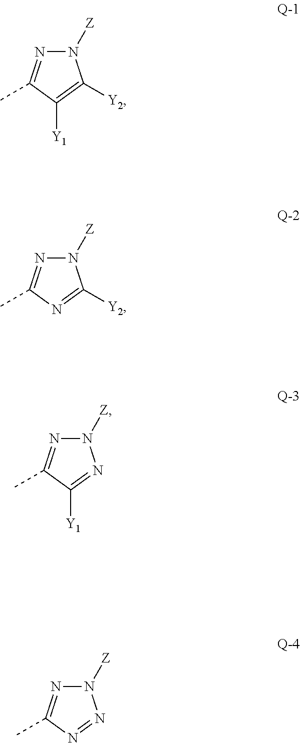
in which the dotted line is the bond to the nitrogen in the NR³ group,
Y₁ is a radical from the group of hydrogen, halogen, cyano, nitro, amino, C₁-C₆-alkylamino, di(C₁-C₆)alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, SH, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di(C₁-C₆)alkylaminocarbonyl,
Y₂ is a radical from the group of hydrogen, halogen, cyano, nitro, amino, C₁-C₆-alkylamino, di(C₁-C₆)alkylamino, hydroxyl, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, SH, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di(C₁-C₆)alkylaminocarbonyl,
in the case that Q=Q-1, Y₁ and Y₂ together with the carbon atoms to which they are bonded form a phenyl ring or a 5- to 7-membered heteroaromatic ring which is optionally substituted by one or more identical or different substituents M¹,
M¹ is halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl and C₁-C₆-alkoxy-C₁-C₆-alkyl,
Z is a radical from the group of hydrogen, cyano, nitro, L¹(=G)C*, L¹O(=G)C*, L³L⁴N(=G)C*, L⁵(=E)CL²N(=G)C*, L⁵(=O)ₙSL²N(=G)C*, L⁵O(=E)CL²N(=G)C*, L¹L⁵N(=E)CL²N(=G)C*, L¹(=O)ₙS*, L³L⁴N(═O)ₙS*, heterocyclyl, aryl, hetaryl, singly or multiply, identically or differently, M²-substituted C₁-C₈-alkyl, C₃-C₈-cycloalkyl, C₂-C₈-alkenyl, C₅-C₈-cycloalkenyl, C₃-C₈-alkynyl, singly or multiply, identically or differently, M⁶-substituted L⁵C(=E)L²N(=G)C—C₁-C₈-alkyl*, L⁵(=O)ₙSL²N(=G)C—C₁-C₈-alkyl*, L⁵OC(=E)L²N(=G)C—C₁-C₈-alkyl*, L¹L⁵NC(=E)L²N(=G)C—C₁-C₈-alkyl*, L¹(=O)ₙS—C₁-C₈-alkyl*, L³O(═O)ₙS—C₁-C₈-alkyl*, L³L⁴N(═O)ₙS—C₁-C₈-alkyl*,
M² is halogen, cyano, nitro, NL⁹L¹⁰, OL⁹, SL⁹, L¹(=G)C*, L³O(=G)C*, L³L⁴N(=G)C*, heterocyclyl, aryl, hetaryl, optionally singly or multiply, identically or differently, M³-substituted C₁-C₆-alkyl, C₃-C₆-cycloalkyl, C₂-C₆-alkenyl, C₅-C₆-cycloalkenyl, C₃-C₆-alkynyl, C₁-C₆-alkoxy,
M³ is halogen, cyano, nitro, heterocyclyl, aryl, hetaryl, NL⁹L¹⁰, OL⁹, SL⁹, C₁-C₆-alkyl, C₁-C₆-haloalkyl, C₃-C₆-cycloalkyl, C₃-C₆-halocycloalkyl, C₂-C₆-alkenyl, C₃-C₆-alkynyl, C₁-C₆-alkoxy, C₁-C₆-haloalkoxy, L¹(=G)C*, L³O(=G)C*, L³L⁴N(=G)C*,
G and E are each independently oxygen or sulphur,
n is 1 or 2,
L¹ and L⁵ are each independently heterocyclyl, aryl, hetaryl, optionally singly or multiply, identically or differently, M⁴-substituted C₁-C₆-alkyl, C₃-C₆-cycloalkyl, C₃-C₆-cycloalkyl-C₁-C₆-alkyl, C₂-C₆-alkenyl, C₃-C₆-alkynyl,
M⁴ is halogen, cyano, nitro, heterocyclyl, aryl, hetaryl, C₁-C₆-haloalkyl, C₁-C₆-alkoxy, C₁-C₆-haloalkoxy, NL⁹L¹⁰, OL⁹, SL⁹,
L² is hydrogen, aryl, hetaryl, optionally singly or multiply, identically or differently, M⁵-substituted C₁-C₆-alkyl, C₃-C₆-cycloalkyl, C₃-C₆-cycloalkyl-C₁-C₆-alkyl, C₂-C₆-alkenyl, C₃-C₆-alkynyl,
L³ and L⁴ are each independently hydrogen, heterocyclyl, aryl, hetaryl, optionally singly or multiply, identically or differently, M⁵-substituted C₁-C₆-alkyl, C₃-C₆-cycloalkyl, C₃-C₆-cycloalkyl-C₁-C₆-alkyl, C₂-C₆-alkenyl, C₃-C₆-alkynyl,
M⁵ is halogen, cyano, nitro, heterocyclyl, aryl, hetaryl, C₁-C₆-haloalkyl, C₁-C₆-alkoxy, C₁-C₆-haloalkoxy, C₁-C₆-alkoxycarbonyl, NL⁹L¹⁰, OL⁹, SL⁹,
M⁶ is halogen, cyano, nitro, NL⁹L¹⁰, OL⁹, SL⁹, L¹(=G)C*, L³O(=G)C*, L³L⁴N(=G)C*, heterocyclyl, aryl, hetaryl, C₁-C₆-alkyl, C₁-C₆-haloalkyl, C₃-C₆-cycloalkyl, C₃-C₆-halocycloalkyl, C₃-C₆-cycloalkyl-C₁-C₆-alkyl, C₃-C₆-halocycloalkyl-C₁-C₆-alkyl, C₂-C₆-alkenyl, C₅-C₆-cycloalkenyl, C₃-C₆-alkynyl, C₁-C₆-alkoxy-C₁-C₆-alkyl, C₁-C₆-haloalkoxy-C₁-C₆-alkyl,
L⁹ and L¹⁰ are each independently hydrogen, C₁-C₆-alkyl, C₁-C₆-haloalkyl, C₃-C₆-cycloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₂-C₆-alkenyl, C₂-C₆-haloalkenyl, C₃-C₆-alkynyl, C₁-C₆-alkoxy-C₁-C₆-alkyl, C₁-C₆-haloalkoxy-C₁-C₆-alkyl, C₁-C₆-alkylcarbonyl, C₂-C₆-alkenylcarbonyl, C₃-C₆-alkynylcarbonyl, C₁-C₆-haloalkylcarbonyl, C₃-C₆-cycloalkylcarbonyl, C₃-C₆-cycloalkyl-C₁-C₆-alkylcarbonyl, C₂-C₆-haloalkenylcarbonyl, C₃-C₆-halocycloalkylcarbonyl, C₁-C₆-alkoxycarbonyl, C₁-C₆-alkoxy-C₁-C₆-alkylcarbonyl, C₁-C₆-haloalkoxycarbonyl, C₃-C₆-cycloalkoxycarbonyl, aminocarbonyl, C₁-C₆-alkylaminocarbonyl, di(C₁-C₆)alkylaminocarbonyl, C₃-C₆-cycloalkylaminocarbonyl, C₃-C₆-cycloalkyl-C₁-C₆-alkylaminocarbonyl, aryl, hetaryl, heterocyclyl, aryl-C₁-C₆-alkyl, hetaryl-C₁-C₆-alkyl,
in which the heterocyclyl, aryl and hetaryl radicals (including as part of a larger unit such as aryloxy, hetarylalkyl etc.) are optionally mono- or polysubstituted, identically or differently, by halogen, cyano, nitro, SH, hydroxyl, C₁-C₆-alkyl, C₂-C₆-alkenyl, C₃-C₆-alkynyl, C₃-C₆-cycloalkyl, C₁-C₆-haloalkyl, cyano-C₁-C₆-alkyl, C₂-C₆-haloalkenyl, C₃-C₆-haloalkynyl, C₁-C₆-alkoxy, C₂-C₆-alkenyloxy, C₃-C₆-alkynyloxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₃-C₆-cycloalkyl-C₁-C₆-alkyl, C₃-C₆-halocycloalkyl-C₁-C₆-alkyl, amino, C₁-C₆-alkylamino, di(C₁-C₆)alkylamino, C₃-C₆-cycloalkylamino, C₃-C₆-cycloalkyl-C₁-C₆-alkylamino, C₁-C₆-alkylcarbonylamino, C₁-C₆-haloalkylcarbonylamino, C₁-C₆-cycloalkylcarbonylamino, CO₂H, SO₃H, CONH₂, SO₂NH₂, C₁-C₆-alkylcarbonyl, C₂-C₆-alkenylcarbonyl, C₃-C₆-alkynylcarbonyl, C₁-C₆-haloalkylcarbonyl, C₃-C₆-cycloalkylcarbonyl, C₃-C₆-cycloalkyl-C₁-C₆-alkylcarbonyl, C₂-C₆-haloalkenylcarbonyl, C₃-C₆-halocycloalkylcarbonyl, C₁-C₆-alkoxycarbonyl, C₁-C₆-alkoxy-C₁-C₆-alkylcarbonyl, C₁-C₆-haloalkoxycarbonyl, C₃-C₆-cycloalkoxycarbonyl, C₁-C₆-alkylthio, C₁-C₆-haloalkylthio, C₁-C₆-alkylsulphinyl, C₁-C₆-haloalkylsulphinyl, C₁-C₆-alkylsulphonyl, C₁-C₆-haloalkylsulphonyl, C₁-C₆-alkylaminosulphonyl, C₁-C₆-haloalkylaminosulphonyl, di(C₁-C₆)alkylaminosulphonyl, C₁-C₆-alkylaminocarbonyl, di(C₁-C₆)alkylaminocarbonyl, C₁-C₆-alkylcarbonyloxy, C₁-C₆-haloalkylcarbonyloxy, C₁-C₆-alkoxycarbonyloxy, C₁-C₆-haloalkoxycarbonyloxy, aryl, hetaryl, aryloxy, hetaryloxy,
and/or a salt and/or N-oxide thereof capable of being used for control of one or more animal pests.

2. Compound of formula (I) 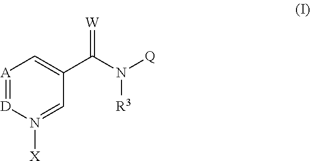
in which
A is N or CR¹,
D is N or CR², where A and D are not both N,
R¹ is a radical from the group of hydrogen, halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl,
R² is a radical from the group of hydrogen, halogen, cyano, nitro, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl,
X is a free electron pair or oxygen,
W is oxygen, sulphur or NR⁴,
R³ is a radical from the group of hydrogen, cyano, hydroxyl, C₁-C₆-alkyl, C₃-C₆-cycloalkyl, C₁-C₆-haloalkyl, C₃-C₆-halocycloalkyl, C₂-C₆-alkenyl, C₂-C₆-haloalkenyl, C₃-C₆-alkynyl, C₃-C₆-haloalkynyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, C₁-C₆-alkylcarbonyl, C₂-C₆-alkenylcarbonyl, C₃-C₆-alkynylcarbonyl, C₁-C₆-haloalkylcarbonyl, C₃-C₆-cycloalkylcarbonyl, C₃-C₆-cycloalkyl-C₁-C₆-alkylcarbonyl, C₂-C₆-haloalkenylcarbonyl, C₃-C₆-halocycloalkylcarbonyl, C₁-C₆-alkoxy-C₁-C₆-alkylcarbonyl, C₁-C₆-alkoxycarbonyl, C₁-C₆-haloalkoxycarbonyl, C₁-C₆-alkylsulphonyl, C₁-C₆-haloalkylsulphonyl, C₁-C₆-alkylaminocarbonyl, di(C₁-C₆)alkylaminocarbonyl, C₁-C₆-alkylcarbonyloxy, C₁-C₆-alkoxycarbonyloxy, aryl, hetaryl, arylcarbonyl, hetarylcarbonyl, aryl-C₁-C₆-alkyl, hetaryl-C₁-C₆-alkyl, aryl-C₁-C₆-alkylcarbonyl, hetaryl-C₁-C₆-alkylcarbonyl, where the aryl, hetaryl, aryl-C₁-C₆-alkyl, hetaryl-C₁-C₆-alkyl, arylcarbonyl, hetarylcarbonyl, aryl-C₁-C₆-alkylcarbonyl, hetaryl-C₁-C₆-alkylcarbonyl substituents are optionally mono- or polysubstituted, identically or differently, by halogen, cyano, nitro, hydroxyl, C₁-C₆-alkyl, C₂-C₆-alkenyl, C₃-C₆-alkynyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl,
R⁴ is a radical from the group of hydrogen, cyano, nitro, hydroxyl, C₁-C₆-alkyl, C₃-C₆-cycloalkyl, C₁-C₆-haloalkyl, C₃-C₆-halocycloalkyl, C₂-C₆-alkenyl, C₂-C₆-haloalkenyl, C₃-C₆-alkynyl, C₃-C₆-haloalkynyl, C₃-C₆-cycloalkyl-C₁-C₆-alkyl, C₃-C₆-halocycloalkyl-C₁-C₆-alkyl, cyano-C₁-C₆-alkyl, C₁-C₆-alkoxy, C₁-C₆-haloalkoxy, C₁-C₆-alkoxy-C₁-C₆-alkyl, C₁-C₆-haloalkoxy-C₁-C₆-alkyl, C₁-C₆-alkylcarbonyl, C₁-C₆-haloalkylcarbonyl, C₁-C₆-alkoxycarbonyl, C₁-C₆-alkylcarbonyloxy, C₁-C₆-alkylsulphonyl, C₁-C₆-haloalkylsulphonyl, aryl, hetaryl, aryl-C₁-C₆-alkyl, hetaryl-C₁-C₆-alkyl, arylcarbonyl, hetarylcarbonyl, where the aryl, hetaryl, aryl-C₁-C₆-alkyl, hetaryl-C₁-C₆-alkyl, arylcarbonyl, hetarylcarbonyl substituents are optionally mono- or polysubstituted, identically or differently, by halogen, cyano, nitro, hydroxyl, C₁-C₆-alkyl, C₂-C₆-alkenyl, C₃-C₆-alkynyl, C₃-C₆-cycloalkyl, C₁-C₆-haloalkyl, C₁-C₆-alkoxy, C₁-C₆-haloalkoxy, C₃-C₆-halocycloalkyl, C₁-C₆-alkoxy-C₁-C₆-alkyl,
Q is one of the Q-1 to Q-4 radicals: 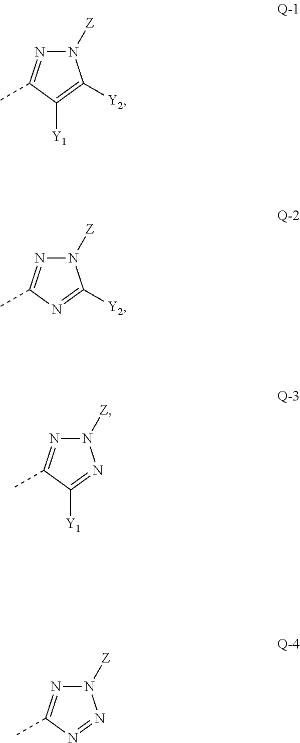
in which the dotted line is the bond to the nitrogen in the NR³ group,
Y₁ is a radical from the group of hydrogen, halogen, cyano, nitro, C₁-C₆-alkylamino, di(C₁-C₆)alkylamino, COOH, C₁-C₆-alkyl, C₃-C₆-cycloalkyl, C₁-C₆-halo alkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di(C₁-C₆)alkylaminocarbonyl,
Y₂ is a radical from the group of hydrogen, halogen, cyano, nitro, C₁-C₆-alkylamino, di(C₁-C₆)alkylamino, COOH, C₁-C₆-alkyl, C₃-C₆-cycloalkyl, C₁-C₆-haloalkyl, C₁-C₆-alkoxy, C₁-C₆-haloalkoxy, C₃-C₆-halocycloalkyl, C₃-C₆-halocycloalkyl-C₁-C₆-alkyl, C₁-C₆-alkoxy-C₁-C₆-alkyl, C₁-C₆-haloalkoxy-C₁-C₆-alkyl, C₁-C₆-alkoxy-C₁-C₆-alkyloxy, cyano-C₁-C₆-alkyl, C₃-C₆-cycloalkyl-C₁-C₆-alkyl, C₂-C₆-alkenyl, C₃-C₆-alkynyl, SH, C₁-C₆-alkylthio, C₁-C₆-alkylcarbonyl, C₃-C₆-cycloalkylcarbonyl, C₂-C₆-alkenylcarbonyl, C₁-C₆-haloalkylcarbonyl, C₁-C₆-alkoxycarbonyl, C₁-C₆-alkylsulphonyl, C₁-C₆-haloalkylsulphonyl, C₁-C₆-alkylaminocarbonyl, di(C₁-C₆)alkylaminocarbonyl,
Z is a radical from the group of hydrogen, cyano, nitro, L¹(=G)C*, L¹O(=G)C*, L³L⁴N(=G)C*, L¹(=E)CL²N(=G)C*, L⁵S(═O)ₙL²N(=G)C*, L⁵O(=E)CL²N(=G)C*, L¹L⁵N(=E)CL²N(=G)C*, L¹(=O)ₙS*, L³L⁴N(═O)ₙS*, heterocyclyl, aryl, hetaryl, singly or multiply, identically or differently, M²-substituted C₁-C₈-alkyl, C₃-C₈-cycloalkyl, C₂-C₈-alkenyl, C₅-C₈-cycloalkenyl, C₃-C₈-alkynyl, singly or multiply, identically or differently, M⁶-substituted L⁵C(=E)L²N(=G)C—C₁-C₈-alkyl*, L⁵S(═O)ₙL²N(=G)C—C₁-C₈-alkyl*, L⁵OC(=E)L²N(=G)C—C₁-C₈-alkyl*, L¹L⁵NC(=E)L²N(=G)C—C₁-C₈-alkyl*, L¹(=O)ₙS—C₁-C₈-alkyl*, L³O(═O)ₙS—C₁-C₈-alkyl*, L³L⁴N(═O)ₙS—C₁-C₈-alkyl*,
with the proviso that, when Q is Q-4, Z is not simultaneously hydrogen or unsubstituted C₁-C₈-alkyl, and with the proviso that Z is not hydrogen when W is NOH, and with the proviso that, when Q is Q-2, Z is not simultaneously hydrogen,
M² is halogen, cyano, nitro, NL⁹L¹⁰, OL⁹, SL⁹, L¹(=G)C*, L³O(=G)C*, L³L⁴N(=G)C*, heterocyclyl, aryl, hetaryl, optionally singly or multiply, identically or differently, M³-substituted C₁-C₆-alkyl, C₃-C₆-cycloalkyl, C₂-C₆-alkenyl, C₅-C₆-cycloalkenyl, C₃-C₆-alkynyl, C₁-C₆-alkoxy,
M³ is halogen, cyano, nitro, heterocyclyl, aryl, hetaryl, NL⁹L¹⁰, OL⁹, SL⁹, C₁-C₆-alkyl, C₁-C₆-haloalkyl, C₃-C₆-cycloalkyl, C₃-C₆-halocycloalkyl, C₂-C₆-alkenyl, C₃-C₆-alkynyl, C₁-C₆-alkoxy, C₁-C₆-haloalkoxy, L¹(=G)C*, L³O(=G)C*, L³L⁴N(=G)C*, G and E are each independently oxygen or sulphur,
n is 1 or 2,
L¹ and L⁵ are each independently heterocyclyl, aryl, hetaryl, optionally singly or multiply, identically or differently, M⁴-substituted C₁-C₆-alkyl, C₃-C₆-cycloalkyl, C₃-C₆-cycloalkyl-C₁-C₆-alkyl, C₂-C₆-alkenyl, C₃-C₆-alkynyl,
M⁴ is halogen, cyano, nitro, heterocyclyl, aryl, hetaryl, C₁-C₆-haloalkyl, C₁-C₆-alkoxy, C₁-C₆-haloalkoxy, NL⁹L¹⁰, OL⁹, SL⁹,
L² is hydrogen, aryl, hetaryl, optionally singly or multiply, identically or differently, M⁵-substituted C₁-C₆-alkyl, C₃-C₆-cycloalkyl, C₃-C₆-cycloalkyl-C₁-C₆-alkyl, C₂-C₆-alkenyl, C₃-C₆-alkynyl,
L³ and L⁴ are each independently hydrogen, heterocyclyl, aryl, hetaryl, optionally singly or multiply, identically or differently, M⁵-substituted C₁-C₆-alkyl, C₃-C₆-cycloalkyl, C₃-C₆-cycloalkyl-C₁-C₆-alkyl, C₂-C₆-alkenyl, C₃-C₆-alkynyl,
M⁵ is halogen, cyano, nitro, heterocyclyl, aryl, hetaryl, C₁-C₆-haloalkyl, C₁-C₆-alkoxy, C₁-C₆-haloalkoxy, C₁-C₆-alkoxycarbonyl, NL⁹L¹⁰, OL⁹, SL⁹,
M⁶ is halogen, cyano, nitro, NL⁹L¹⁰, OL⁹, SL⁹, L¹(=G)C*, L³O(=G)C*, L³L⁴N(=G)C*, heterocyclyl, aryl, hetaryl, C₁-C₆-alkyl, C₁-C₆-haloalkyl, C₃-C₆-cycloalkyl, C₃-C₆-halocycloalkyl, C₃-C₆-cycloalkyl-C₁-C₆-alkyl, C₃-C₆-halocycloalkyl-C₁-C₆-alkyl, C₂-C₆-alkenyl, C₅-C₆-cycloalkenyl, C₃-C₆-alkynyl, C₁-C₆-alkoxy-C₁-C₆-alkyl, C₁-C₆-haloalkoxy-C₁-C₆-alkyl,
L⁹ and L¹⁰ are each independently hydrogen, C₁-C₆-alkyl, C₁-C₆-haloalkyl, C₃-C₆-cycloalkyl, C₃-C₆-halocycloalkyl, C₃-C₆-cycloalkyl-C₁-C₆-alkyl, C₃-C₆-halocycloalkyl-C₁-C₆-alkyl, cyano-C₁-C₆-alkyl, C₂-C₆-alkenyl, C₂-C₆-haloalkenyl, C₃-C₆-alkynyl, C₁-C₆-alkoxy-C₁-C₆-alkyl, C₁-C₆-haloalkoxy-C₁-C₆-alkyl, C₁-C₆-alkylcarbonyl, C₂-C₆-alkenylcarbonyl, C₃-C₆-alkynylcarbonyl, C₁-C₆-haloalkylcarbonyl, C₃-C₆-cycloalkylcarbonyl, C₃-C₆-cycloalkyl-C₁-C₆-alkylcarbonyl, C₂-C₆-haloalkenylcarbonyl, C₃-C₆-halocycloalkylcarbonyl, C₁-C₆-alkoxycarbonyl, C₁-C₆-alkoxy-C₁-C₆-alkylcarbonyl, C₁-C₆-haloalkoxycarbonyl, C₃-C₆-cycloalkoxycarbonyl, aminocarbonyl, C₁-C₆-alkylaminocarbonyl, di(C₁-C₆)alkylaminocarbonyl, C₃-C₆-cycloalkylaminocarbonyl, C₃-C₆-cycloalkyl-C₁-C₆-alkylaminocarbonyl, aryl, hetaryl, heterocyclyl, aryl-C₁-C₆-alkyl, hetaryl-C₁-C₆-alkyl,
in which, unless stated otherwise, the heterocyclyl, aryl and hetaryl radicals (including as part of a larger unit such as aryloxy, hetarylalkyl etc.) are optionally mono- or polysubstituted, identically or differently, by halogen, cyano, nitro, SH, hydroxyl, C₁-C₆-alkyl, C₂-C₆-alkenyl, C₃-C₆-alkynyl, C₃-C₆-cycloalkyl, C₁-C₆-haloalkyl, cyano-C₁-C₆-alkyl, C₂-C₆-haloalkenyl, C₃-C₆-haloalkynyl, C₁-C₆-alkoxy, C₂-C₆-alkenyloxy, C₃-C₆-alkynyloxy, C₁-C₆-haloalkoxy, C₃-C₆-halocycloalkyl, C₁-C₆-alkoxy-C₁-C₆-alkyl, C₁-C₆-haloalkoxy-C₁-C₆-alkyl, C₃-C₆-cycloalkyl-C₁-C₆-alkyl, C₃-C₆-halocycloalkyl-C₁-C₆-alkyl, amino, C₁-C₆-alkylamino, di(C₁-C₆)alkylamino, C₃-C₆-cycloalkylamino, C₃-C₆-cycloalkyl-C₁-C₆-alkylamino, C₁-C₆-alkylcarbonylamino, C₁-C₆-haloalkylcarbonylamino, C₁-C₆-cycloalkylcarbonylamino, CO₂H, SO₃H, CONH₂, SO₂NH₂, C₁-C₆-alkylcarbonyl, C₂-C₆-alkenylcarbonyl, C₃-C₆-alkynylcarbonyl, C₁-C₆-haloalkylcarbonyl, C₃-C₆-cycloalkylcarbonyl, C₃-C₆-cycloalkyl-C₁-C₆-alkylcarbonyl, C₂-C₆-haloalkenylcarbonyl, C₃-C₆-halocycloalkylcarbonyl, C₁-C₆-alkoxycarbonyl, C₁-C₆-alkoxy-C₁-C₆-alkylcarbonyl, C₁-C₆-haloalkoxycarbonyl, C₃-C₆-cycloalkoxycarbonyl, C₁-C₆-alkylthio, C₁-C₆-haloalkylthio, C₁-C₆-alkylsulphinyl, C₁-C₆-haloalkylsulphinyl, C₁-C₆-alkylsulphonyl, C₁-C₆-haloalkylsulphonyl, C₁-C₆-alkylaminosulphonyl, C₁-C₆-haloalkylaminosulphonyl, di(C₁-C₆)alkylaminosulphonyl, C₁-C₆-alkylaminocarbonyl, di(C₁-C₆)alkylaminocarbonyl, C₁-C₆-alkylcarbonyloxy, C₁-C₆-haloalkylcarbonyloxy, C₁-C₆-alkoxycarbonyloxy, C₁-C₆-haloalkoxycarbonyloxy, aryl, hetaryl, aryloxy, hetaryloxy,
with the proviso that, when Y₂ is methyl and Z is phenyl-C₁-C₆-alkyl, the phenyl ring in the ortho position is not substituted by alkoxy or by arylalkoxy, with the proviso that Z is not 3,5-dimethyl-1,2-oxazol-4-ylmethyl when A and D are each CH and, at the same time, X is a free electron pair, W is oxygen, Q is Q-1 and R³, Y₁ and Y₂ are each hydrogen, and with the proviso that compounds of the formulae (G1), (G2) and (G4) are excluded.

3. Composition, comprising a content of at least one compound of formula (I) according to claim 1 and one or more customary extenders and/or surfactants.

4. Composition, comprising a content of at least one compound of formula (I) according to claim 2 and one or more customary extenders and/or surfactants.

5. Method for controlling one or more pests, comprising allowing a compound of formula (I) according to claim 1 to act on the pests and/or a habitat thereof.

6. A compound of formula (I) according to claim 2 capable of being used for controlling one or more pests.
